(19) United States
(12) Patent Application Publication
(10) Pub. No.: US 2003/0228570 A1
(43) Pub. Date: Dec. 11, 2003

(54) Methods of diagnosis of Hepatitis C infection, compositions and methods of screening for modulators of Hepatitis C infection

(75) Inventors: Edward Yat Wah Tom, Sacramento, CA (US); Albert Zlotnik, Palo Alto, CA (US)

Correspondence Address: Albert P. Halluin, HOWREY SIMON ARNOLD & WHITE, LLP, 301 Ravenswood Avenue, Box No. 34, Menlo Park, CA, 94025, US

(73) Assignee: Eos Biotechnology, Inc., South San Francisco, CA

(21) Appl. No.: 10/366,435

(22) Filed: Feb. 12, 2003

Related U.S. Application Data
(63) Continuation of application No. 10/206,473, filed on Jul. 24, 2002, now abandoned.
(60) Provisional application No. 60/366,782, filed on Mar. 21, 2002.
(60) Provisional application No. 60/308,188, filed on Jul. 26, 2001.

(51) Int. Cl. C12Q 1/70; C12Q 1/68; C07H 21/04; C07K 14/02; C07K 16/08; C12P 21/02; C12N 5/06

(52) U.S. Cl. 435/5; 435/6; 435/69.3; 435/320.1; 435/325; 530/350; 530/388.3; 536/23.72

(57) ABSTRACT

Described herein are genes whose expression are up-regulated or down-regulated during the course of Hepatitis C infection, or distinction between treatment response. Related methods and compositions that can be used for diagnosis and treatment of Hepatitis C infection and/or its secondary consequences are disclosed. Also described herein are methods that can be used to identify modulators of Hepatitis C infection and/or its secondary consequences.

### CROSS-REFERENCES TO RELATED APPLICATIONS

The present invention is related to U.S. S No. 60/308,188, filed Jul. 26, 2001; and U.S. S No. 60/366,782, filed Mar. 21, 2002, each of which is incorporated herein by reference.

### FIELD OF THE INVENTION

The invention relates to the identification of nucleic acid and protein expression profiles and nucleic acids, products, and antibodies thereto that are involved in Hepatitis C infection; and to the use of such expression profiles to identify compositions relevant in the diagnosis, prognosis, and therapy of Hepatitis C infection and its secondary consequences. The invention further relates to methods for identifying and using agents and/or targets that inhibit Hepatitis C infection or the effects therefrom.

### BACKGROUND OF THE INVENTION

Hepatitis C virus (HCV) infects an estimated 170 million persons worldwide and thus, represents a viral pandemic that is five times as widespread as infection with the Human immunodeficiency-1 virus (HIV-1). In the United States alone an estimated 2.7 million Americans have active HCV infections.

Sexual transmission of the virus is an inefficient means of infection, rather, the factors most strongly associated with infection are injection drug use and before 1990, receipt of blood transfusion. In some cases no risk factors can be identified. Fortunately, introduction in 1990 and 1992 of improved blood screening measures, based on the detection of HCV antibodies, dramatically decreased the risk of transfusion associated HCV infection.

HCV is a positive strand RNA virus that belongs to the family of Flavivirus. The natural targets of HCV are the hepatocytes and possibly also B lymphocytes. The genome is about 9400 nucleotides in length and encodes a single large polyprotein of about 3000 amino acids which undergoes proteolysis to form the mature viral proteins. Structural components include the core and two envelope proteins. Two of the regions of the envelope E2 protein, designated hypervariable regions 1 and 2 have an extremely high rate of mutation, believed to be the result of selective pressure by virus specific antibodies. Because the virus is highly mutable and evolves over the course of infection, therapies directed solely at targeting an immune response toward the virus can be ineffective in clearing the viral load. See, e.g., Lauer and Walker (2001) “Hepatitis C Virus Infection” N. E. J. Med. 345:41-52.

In most persons who become infected with HCV, viremia persists indefinitely and is accompanied by variable degrees of hepatic inflammation and fibrosis. HCV infection is rarely diagnosed during the acute phase of infection when the possibility of viral clearance is greatest. Clinical manifestations of HCV infection usually occur between 2-26 weeks after exposure to HCV, but the majority of persons are asymptomatic. The symptoms that do sometimes accompany acute HCV infection are usually mild and, when present, consist of jaundice, malaise, and nausea. In most cases, acute infection leads to chronic infection which is typically characterized by a prolonged period in which there are no symptoms. Once chronic infection has been established, spontaneous clearance of viremia is rare.

Viral clearance is associated with the development and persistence of strong virus-specific responses by cytotoxic T lymphocytes and helper T cells. The relatively weak response of cytotoxic T lymphocytes in persons with chronic HCV infection while insufficient to contain viremia and genetic evolution of the virus is still sufficient to cause collateral damage through the elaboration of inflammatory cytokines in the liver. The constant low level inflammatory response leads to hepatitis in most cases of chronic infection and also to some degree of fibrosis which may, in turn, be accompanied by relatively non-specific symptoms such as fatigue. Cirrhosis develops in 15-20% of those individuals who are chronically infected with HCV and these individuals are at high risk for developing severe complications, such as hepatic carcinoma. In fact, once cirrhosis is established, the risk of hepatocellular carcinoma is approximately 1-4% per year.

In addition to hepatic disease, there are important extrahepatic manifestations of HCV infection. Most of these syndromes are associated with autoimmune or lymphoproliferative states and may be related to the possibility that HCV is able to replicate in lymphoid cells. For example, a higher incidence of non-Hodgkin's Lymphoma has been observed in HCV infection.

Clearly, a need exists for the identification of novel therapeutic targets and diagnostic markers of HCV infection. Early diagnosis improves the chances that an individual will be able to clear the virus before infection becomes chronic. But, since most infections do become chronic, it is worthwhile to pursue alternative therapies which can be directed at alleviating the continuous low level inflammatory response that accompanies HCV infection and other secondary consequences of HCV infection such as liver fibrosis, which in turn, leads ultimately to cirrhosis and hepatocellular carcinoma.

Advances in molecular medicine will facilitate elucidation of a role for novel proteins and compounds in disease states. Identification of therapeutic targets and diagnostic markers is essential for improving the current treatment of Hepatitis C infected patients. Accordingly, provided herein are molecular targets for therapeutic intervention in all aspects of Hepatitis C infection. Additionally, provided herein are methods that can be used in diagnosis and prognosis of Hepatitis C infection and/or it secondary consequences. Further provided are methods that can be used to screen candidate bioactive agents for the ability to modulate Hepatitis C infection and/or its secondary consequences.

The current therapeutic regimen for HCV infection is treatment with interferon alpha (IFN-α). See, e.g., Berkow, The Merck Manual. In chronic hepatitis C, interferon-α at a dosage of 3 million IU subcutaneous three times weekly initially suppresses inflammation in about 50% of patients. Responders are usually treated for 12 months, but most relapse when treatment is stopped; successful long-term disease suppression is only about 20 to 25% overall. Response depends in part on the viral load, viral genotype, and histological state of the disease. Combination therapy with interferon plus oral Ribavirin™ (1200 mg daily in 2 divided doses) may give a higher rate of sustained response. In addition to having limited efficacy, interferon is expensive, must be given by injection, produces bothersome flu-like side effects in most patients, and induces more serious side effects in a minority of cases. Treatment should be supervised by a specialist. Other antiviral and immunomodulatory drugs against HCV have been evaluated or are being studied, but none has shown much promise except the combination of interferon plus ribavirin™.

Thus, means to early diagnose infection, and to identify patients who are likely to respond to treatment with IFN-α would be useful. Prognosis of response to treatment will minimize the occurrence of negative side effects in those patients who will not respond, and will allow early application of alternative therapies early in the infection. The present invention provides these and many other important capabilities.

### SUMMARY OF THE INVENTION

The present invention therefore provides nucleotide sequences of genes that are up- and down-regulated in Hepatitis C infected cells and in cells affected indirectly by Hepatitis C infection. Such genes are useful for diagnostic purposes, and also as targets for screening for therapeutic compounds that modulate Hepatitis C infection and/or its secondary consequences, such as hormones or antibodies. Other aspects of the invention will become apparent to the skilled artisan by the following description of the invention.

In one aspect, the present invention provides a method of detecting a RNA transcript associated with Hepatitis C infection, in a cell from a patient, the method comprising contacting a biological sample from the patient with a polynucleotide that selectively hybridizes to a sequence at least 80% identical to a sequence as shown in Tables 1A-15.

In one embodiment, the present invention provides a method of determining the level of a Hepatitis C infection associated transcript in a cell from a patient.

In one embodiment, the present invention provides a method of detecting a Transcript associated with Hepatitis C infection in a cell from a patient, the method comprising contacting a biological sample from the patient with a polynucleotide that selectively hybridizes to a sequence at least 80% identical to a sequence as shown in Tables 1A-15.

In one embodiment, the polynucleotide selectively hybridizes to a sequence at least 95% identical to a sequence as shown in Tables 1A-15.

In one embodiment, the biological sample is a tissue sample, e.g., a liver biopsy. In another embodiment, the biological sample comprises isolated nucleic acids, e.g., mRNA.

In one embodiment, the polynucleotide is labeled, e.g., with a fluorescent label.

In one embodiment, the polynucleotide is immobilized on a solid surface.

In one embodiment, the patient is undergoing a therapeutic regimen to treat Hepatitis C infection.

In one embodiment, the patient is a primate or human.

In one embodiment, the Hepatitis C associated transcript is mRNA.

In one embodiment, the method further comprises the step of amplifying nucleic acids before the step of contacting the biological sample with the polynucleotide.

In another aspect, the present invention provides a method of monitoring the efficacy of a therapeutic treatment for Hepatitis C infection and/or its secondary consequences, the method comprising steps of: (i) providing a biological sample from a patient undergoing the therapeutic treatment; and (ii) determining the level of a Transcript associated with Hepatitis C infection in the biological sample by contacting the biological sample with a polynucleotide that selectively hybridizes to a sequence at least 80% identical to a sequence as shown in Tables 1A-15, thereby monitoring the efficacy of the therapy. In a further embodiment, the patient has a drug resistant form of Hepatitis C infection.

In one embodiment, the method further comprises a step of: (iii) comparing the level of the RNA transcript associated with Hepatitis C infection to a level of the Transcript associated with Hepatitis C infection in a biological sample from the patient prior to, or earlier in, the therapeutic treatment.

Additionally, provided herein is a method of evaluating the effect of a candidate drug for treating Hepatitis C infection and/or its secondary consequences, comprising administering drug to a patient and removing a cell sample from the patient. The expression profile of the cell is then determined. This method may further comprise comparing the expression profile to an expression profile of a healthy individual or other comparison sample. In a preferred embodiment, said expression profile includes a gene of Tables 1A-15.

In one aspect, the present invention provides an isolated nucleic acid molecule consisting of a polynucleotide sequence as shown in Tables 1A-15.

In one embodiment, an expression vector or cell comprises the isolated nucleic acid.

In one aspect, the present invention provides an isolated polypeptide which is encoded by a nucleic acid molecule having polynucleotide sequence as shown in Tables 1A-15.

In another aspect, the present invention provides an antibody that specifically binds to an isolated polypeptide which is encoded by a nucleic acid molecule having polynucleotide sequence as shown in Tables 1A-15.

In one embodiment, the antibody is conjugated to an effector component, e.g., a fluorescent label, a radioisotope, or a cytotoxic chemical.

In one embodiment, the antibody is an antibody fragment. In another embodiment, the antibody is humanized.

In one aspect, the present invention provides a method of detecting a Hepatitis C infected cell or a cell affected secondarily by Hepatitis C infection in a biological sample from a patient, the method comprising contacting the biological sample with an antibody as described herein.

In another aspect, the present invention provides a method of detecting antibodies specific to Hepatitis C infection in a patient, the method comprising contacting a biological sample from the patient with a polypeptide encoded by a nucleic acid comprising a sequence from Tables 1A-15.

In another aspect, the present invention provides a method for identifying a compound that modulates a Hepatitis C infection-associated polypeptide, the method comprising steps of: (i) contacting the compound with a Hepatitis C infection-associated polypeptide, the polypeptide encoded by a polynucleotide that selectively hybridizes to a sequence at least 80% identical to a sequence as shown in Tables 1A-15; and (ii) determining the functional effect of the compound upon the polypeptide.

In one embodiment, the functional effect is a physical effect, an enzymatic effect, a physiological effect, or a chemical effect.

In one embodiment, the polypeptide is expressed in a eukaryotic host cell or cell membrane. In another embodiment, the polypeptide is recombinant.

In one embodiment, the functional effect is determined by measuring ligand binding to the polypeptide.

In another aspect, the present invention provides a method of inhibiting proliferation of a Hepatitis C infected or a cell secondarily affected by Hepatitis C infection to treat Hepatitis C infection in a patient, the method comprising the step of administering to the subject a therapeutically effective amount of a compound identified as described herein.

In one embodiment, the compound is an antibody, e.g., one or more monoclonal antibodies.

In another aspect, the present invention provides a drug screening assay comprising steps of: (i) administering a test compound to a mammal suffering from a Hepatitis C infection or to a cell sample isolated therefrom; (ii) comparing the level of gene expression of a polynucleotide that selectively hybridizes to a sequence at least 80% identical to a sequence as shown in Tables 1A-15 in a treated cell or mammal with the level of gene expression of the polynucleotide in a control cell sample or mammal, wherein a test compound that modulates the level of expression of the polynucleotide is a candidate for the treatment of Hepatitis C infection and/or its secondary consequences.

In one embodiment, the control is a mammal, e.g., primate, infected with Hepatitis C virus or a cell sample therefrom that has not been treated with the test compound. In another embodiment, the control is a normal cell or mammal.

In one embodiment, the test compound is administered in varying amounts or concentrations. In another embodiment, the test compound is administered for varying time periods. In another embodiment, the comparison can occur before or after addition or removal of the drug candidate.

In one embodiment, the levels of a plurality of polynucleotides that selectively hybridize to a sequence at least 80% identical to a sequence as shown in Tables 1A-15 are individually compared to their respective levels in a control cell sample or mammal. In a preferred embodiment the plurality of polynucleotides is from three to ten.

In another aspect, the present invention provides a method for treating a mammal infected with Hepatitis C virus comprising administering a compound identified by the assay described herein.

In another aspect, the present invention provides a pharmaceutical composition for treating a mammal, e.g., primate, infected with Hepatitis C virus, the composition comprising a compound identified by the assay described herein and a physiologically acceptable excipient.

In one aspect, the present invention provides a method of screening drug candidates by providing a cell expressing a gene that is up- or down-regulated as in a Hepatitis C infection. In one embodiment, a gene is selected from Tables 1A-15. The method further includes adding a drug candidate to the cell and determining the effect of the drug candidate on the expression of the expression profile gene.

In one embodiment, the method of screening drug candidates includes comparing the level of expression in the absence of the drug candidate to the level of expression in the presence of the drug candidate, wherein the concentration of the drug candidate can vary when present, and wherein the comparison can occur after addition or removal of the drug candidate. In a preferred embodiment, the cell expresses at least two or more expression profile genes. The profile genes may each show change, e.g., an increase or decrease.

Also provided is a method of evaluating the effect of a candidate drug for the treatment of Hepatitis C infection and/or its secondary consequences comprising administering the drug to a transgenic animal expressing or over-expressing the Hepatitis C infection modulatory protein, or an animal lacking the Hepatitis C infection modulatory protein, e.g., as a result of a gene knockout.

Moreover, provided herein is a biochip comprising one or more nucleic acid segments of Tables 1A-15, wherein the biochip comprises fewer than 1000 nucleic acid probes. Preferably, at least two nucleic acid segments are included. More preferably, at least three nucleic acid segments are included.

Furthermore, a method of diagnosing a disorder associated with Hepatitis C infection is provided. The method comprises determining the expression of a gene of Tables 1A-15, in a first tissue type of a first individual, and comparing the distribution to the expression of the gene from a second uninfected individual. A difference in the expression indicates that the first individual has a disorder associated with Hepatitis C infection.

In a further embodiment, the biochip also includes a polynucleotide sequence of a gene that is not changed, e.g., up- or down-regulated in Hepatitis C infection.

In one embodiment a method for screening for a bioactive agent capable of interfering with the binding of a Hepatitis C infection modulating protein (Hepatitis C infection modulatory protein) or a fragment thereof and an antibody which binds to said Hepatitis C infection modulatory protein or fragment thereof. In a preferred embodiment, the method comprises combining a Hepatitis C infection modulatory protein or fragment thereof, a candidate bioactive agent, and an antibody which binds to said Hepatitis C infection modulatory protein or fragment thereof. The method further includes determining the binding of said Hepatitis C infection modulatory protein or fragment thereof and said antibody. When there is a change in binding, an agent is identified as an interfering agent. The interfering agent can be an agonist or an antagonist. Preferably, the agent inhibits Hepatitis C infection and/or the secondary consequences of Hepatitis C infection.

Also provided herein are methods of modulating an immune response in an individual, e.g., primate. In one embodiment a method provided herein comprises administering to an individual a composition comprising a Hepatitis C infection modulating protein, or a fragment thereof. In another embodiment, the protein is encoded by a nucleic acid selected from those of Tables 1A-15.

Further provided herein are compositions capable of eliciting an immune response in an individual. In one embodiment, a composition provided herein comprises a Hepatitis C infection modulating protein, preferably encoded by a nucleic acid of Tables 1A-15, or a fragment thereof, and a pharmaceutically acceptable carrier. In another embodiment, said composition comprises a nucleic acid comprising a sequence encoding a Hepatitis C infection modulating protein, preferably selected from the nucleic acids of Tables 1A-15, and a pharmaceutically acceptable carrier.

Also provided are methods of neutralizing the effect of a protein associated with Hepatitis C infection and/or its secondary consequences, or a fragment thereof, comprising contacting an agent specific for said protein with said protein in an amount sufficient to effect neutralization. In another embodiment, the protein is encoded by a nucleic acid selected from those of Tables 1A-15.

In another aspect of the invention, a method of treating an individual infected with Hepatitis C is provided. In one embodiment, the method comprises administering to said individual, e.g., primate, an inhibitor of a Hepatitis C infection modulating protein. In another embodiment, the method comprises administering to a patient, e.g., primate, infected with Hepatitis C virus, an antibody to a Hepatitis C infection modulating protein conjugated to a therapeutic moiety. Such a therapeutic moiety can be a cytotoxic agent or a radioisotope.

### DETAILED DESCRIPTION OF THE INVENTION

In accordance with the objects outlined above, the present invention provides novel methods for diagnosis and prognosis evaluation for Hepatitis C infection and/or its secondary consequences, as well as methods for screening for compositions which modulate Hepatitis C infection and/or its secondary consequences. Markers are identified which correlate with subsets of patients who respond to IFN-α treatment, or with subsets of patients who are retractile (non-responsive) to treatment with standard IFN-α treatment. Also provided are methods for treating Hepatitis C infection and/or its secondary consequences.

Definitions

The term “Hepatitis C infection polynucleotide” or “transcript associated with Hepatitis C infection” refers to nucleic acid polymorphic variants, alleles, mutants, and interspecies homologues isolated from cells involved in Hepatitis C infection and/or its secondary consequences, or those which allow for subsetting of infected patients. The cells from which the nucleic acids are isolated include cells such as hepatocytes and B lymphocytes, that are directly infected by virus, as well as cells that may be indirectly affected by the viral infection such as those cells involved in the immune and inflammatory response to Hepatitis C infection. The terms also refer to nucleic acids that: (1) have a nucleotide sequence with greater than about 60% nucleotide sequence identity, 65%, 70%, 75%, 80%, 85%, 90%, preferably 91%, 92%, 93%, 94%, 95%, 96%, 97%, 98%, or 99% or greater nucleotide sequence identity, preferably over a region of over a region of at least about 25, 50, 100, 200, 500, 1000, or more nucleotides, to a nucleotide sequence of or associated with a gene of Tables 1A-15; or, (2) specifically hybridize under stringent hybridization conditions to a nucleic acid sequence, or the complement thereof, of Tables 1A-15 and conservatively modified variants thereof.

The term “Hepatitis C infection protein” and similar terms refer to polypeptide polymorphic variants, alleles, mutants, and interspecies homologues isolated from cells involved in Hepatitis C infection and its secondary consequences, including ones which allow for subsetting patients, e.g., into responsive or non-responsive subsets. The cells from which the polypeptides are isolated include cells such as hepatocytes and B lymphocytes, that are directly infected by virus, as well as cells that may be indirectly affected by the viral infection such as those cells involved in the immune and inflammatory response to Hepatitis C infection. The terms also refer to polypeptides that: (1) bind to antibodies, e.g., polyclonal antibodies, raised against an immunogen comprising an amino acid sequence encoded by a nucleotide sequence of or associated with a gene of Tables 1A-15, and conservatively modified variants thereof; or (2) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino sequence identity, preferably over a region of over a region of at least about 25, 50, 100, 200, 500, 1000, or more amino acid, to an amino acid sequence encoded by a nucleotide sequence of, or associated with, a gene of Tables 1A-15.

A polynucleotide or polypeptide sequence is typically from a mammal including, but not limited to, primate, e.g., human; rodent, e.g., rat, mouse, hamster; cow, pig, horse, sheep, or other mammal, domestic or livestock. A “Hepatitis C infection polypeptide” and a “Hepatitis C infection polynucleotide,” include both naturally occurring or recombinant forms.

A “full length” Hepatitis C infection protein or nucleic acid refers to a Hepatitis C infection polypeptide or polynucleotide sequence, or a variant thereof, that contains all of the elements normally contained in one or more naturally occurring, wild type Hepatitis C infection polynucleotide or polypeptide sequences. The “full length” may be prior to, or after, various stages of post-translational processing or splicing, including alternative splicing.

“Biological sample” as used herein is a sample of biological tissue or fluid that contains nucleic acids or polypeptides, e.g., of a Hepatitis C infection protein, polynucleotide, or transcript. Such samples include, but are not limited to, tissue isolated from primates, e.g., humans, or rodents, e.g., mice and rats. Biological samples may also include sections of tissues such as biopsy and autopsy samples, frozen sections taken for histologic purposes, archival specimens, blood, plasma, serum, sputum, stool, tears, mucus, hair, skin, etc. Biological samples also include explants and primary and/or transformed cell cultures derived from patient tissues. A biological sample is typically obtained from a eukaryotic organism, most preferably a mammal such as a primate e.g., chimpanzee or human; cow; dog; cat; a rodent, e.g., guinea pig, rat, mouse; rabbit; or a bird; reptile; or fish.

“Providing a biological sample” means to obtain a biological sample for use in methods described in this invention. Most often, this will be done by removing a sample of cells from an animal, but can also be accomplished by using previously isolated cells (e.g., isolated by another person, at another time, and/or for another purpose), or by performing the methods of the invention in vivo. Archival tissues, having treatment and/or outcome history, will be particularly useful.

The terms “identical” or percent “identity,” in the context of two or more nucleic acids or polypeptide sequences, refer to two or more sequences or subsequences that are the same or have a specified percentage of amino acid residues or nucleotides that are the same (e.g., about 60% identity, preferably 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/ or the like). Such sequences are then said to be “substantially identical.” This definition also refers to, or may be applied to, the complement of a test sequence. The definition also includes sequences that have deletions and/or additions, as well as those that have substitutions, as well as naturally occurring, e.g., polymorphic or allelic variants, and man-made variants. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of one of the number of contiguous positions selected from the group consisting typically of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith and Waterman (1981) Adv. Appl. Math. 2:482-489, by the homology alignment algorithm of Needleman and Wunsch (1970) J. Mol. Biol. 48:443-453, by the search for similarity method of Pearson and Lipman (1988) Proc. Nat'l. Acad. Sci. USA 85:2444-2448, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel, et al. (eds. 1995 and supplements) Current Protocols in Molecular Biology Lippincott.

Preferred examples of algorithms that are suitable for determining percent sequence identity and sequence similarity include the BLAST and BLAST 2.0 algorithms, which are described, e.g., in Altschul, et al. (1977) Nuc. Acids Res. 25:3389-3402 and Altschul, et al. (1990) J. Mol. Biol. 215:403-410. BLAST and BLAST 2.0 are used, with the parameters described herein, to determine percent sequence identity for nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul, et al., supra). These initial neighborhood word hits act as seeds for initiating searches to find longer HSPs containing them. The word hits are extended in both directions along each sequence for as far as the cumulative alignment score can be increased. Cumulative scores are calculated using, e.g., for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Nat'l Acad. Sci. USA 89:10915-919) alignments (B) of 50, expectation (E) of 10, M=5, N=−4, and a comparison of both strands.

The BLAST algorithm also performs a statistical analysis of the similarity between two sequences. See, e.g., Karlin and Altschul (1993) Proc. Nat'l. Acad. Sci. USA 90:5873-5787. One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001. Log values may be large negative numbers, e.g., 5, 10, 20, 30, 40, 40, 70, 90, 110, 150, 170, etc.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, e.g., where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequences.

A “host cell” is a naturally occurring cell or a transformed cell that contains an expression vector and supports the replication or expression of the expression vector. Host cells may be cultured cells, explants, cells in vivo, and the like. Host cells may be prokaryotic cells such as E. coli, or eukaryotic cells such as yeast, insect, amphibian, or mammalian cells such as CHO, HeLa, and the like (see, e.g., the American Type Culture Collection catalog or web site, www.atcc.org).

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein or nucleic acid that is the predominant species present in a preparation is substantially purified. In particular, an isolated nucleic acid is separated from some open reading frames that naturally flank the gene and encode proteins other than protein encoded by the gene. The term “purified” in some embodiments denotes that a nucleic acid or protein gives rise to essentially one band in an electrophoretic gel. Preferably, it means that the nucleic acid or protein is at least 85% pure, more preferably at least 95% pure, and most preferably at least 99% pure. “Purify” or “purification” in other embodiments means removing at least one contaminant from the composition to be purified. In this sense, purification does not require that the purified compound be homogenous, e.g., 100% pure.

The terms “polypeptide,” “peptide”, and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an α carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG, and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid which encodes a polypeptide is implicit in a described sequence with respect to the expression product, but not with respect to actual probe sequences.

As to amino acid sequences, one of skill will recognize that individual substitutions, deletions, or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. Typically conservative substitutions include for one another: 1) Alanine (A), Glycine (G); 2) Aspartic acid (D), Glutamic acid (E); 3) Asparagine (N), Glutamine (O); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M). See, e.g., Creighton (1984) Proteins: Structure and Molecular Properties Freeman.

Macromolecular structures such as polypeptide structures can be described in terms of various levels of organization. For a general discussion of this organization, see, e.g., Alberts, et al. (1994) Molecular Biology of the Cell (3d ed.) Garland; and Cantor and Schimmel (1980) Biophysical Chemistry Part I: The Conformation of Biological Macromolecules Freeman. “Primary structure” refers to the amino acid sequence of a particular peptide. “Secondary structure” refers to locally ordered, three dimensional structures within a polypeptide. These structures are commonly known as domains. Domains are portions of a polypeptide that often form a compact unit of the polypeptide and are typically about 25-500 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed, usually by the noncovalent association of independent tertiary units. Anisotropic terms are also known as energy terms.

“Nucleic acid” or “oligonucleotide” or “polynucleotide” or grammatical equivalents used herein means at least two nucleotides covalently linked together. Oligonucleotides are typically from about 5, 6, 7, 8, 9, 10, 12, 15, 25, 30, 40, 50 or more nucleotides in length, up to about 100 nucleotides in length. Nucleic acids and polynucleotides are a polymers of any length, including longer lengths, e.g., 200, 300, 500, 1000, 2000, 3000, 5000, 7000, 10,000, etc. A nucleic acid of the present invention will generally contain phosphodiester bonds, although in some cases, nucleic acid analogs are included that may have alternate backbones, comprising, e.g., phosphoramidate, phosphorothioate, phosphorodithioate, or O-methylphophoroamidite linkages (see Eckstein (1992) Oligonucleotides and Analogues: A Practical Approach Oxford Univ. Press); and peptide nucleic acid backbones and linkages. Other analog nucleic acids include those with positive backbones; non-ionic backbones, and non-ribose backbones, including those described in U.S. Pat. Nos. 5,235,033 and 5,034,506, and Chapters 6 and 7 in Sanghvi and Cook (eds. 1994) Carbohydrate Modifications in Antisense Research ACS Symposium Series 580. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made.

A variety of references disclose such nucleic acid analogs, including, for example, phosphoramidate (Beaucage, et al. (1993) Tetrahedron 49:1925-1963 and references therein; Letsinger (1970) J. Org. Chem. 35:3800-3803; Sprinzl, et al. (1977) Eur. J. Biochem. 81:579-589; Letsinger, et al. (1986) Nucl. Acids Res. 14:3487499; Sawai, et al. (1984) Chem. Lett. 805, Letsinger, et al. (1988) J. Am. Chem. Soc. 110:4470-4471; and Pauwels, et al. (1986) Chemica Scripta 26:141-149), phosphorothioate (Mag, et al. (1991) Nuc. Acids Res. 19:1437-441; and U.S. Pat. No. 5,644,048), phosphorodithioate (Brill, et al. (1989) J. Am. Chem. Soc. 111:2321-322), O-methylphophoroamidite linkages (see Eckstein (1992) Oligonucleotides and Analogues: A Practical Approach, Oxford Univ. Press), and peptide nucleic acid backbones and linkages (see Egholm (1992) J. Am. Chem. Soc. 114:1895-1897; Meier, et al. (1992) Chem. Int. Ed. Engl. 31:1008-1010; Nielsen (1993) Nature 365:566-568; and Carlsson, et al. (1996) Nature 380:207). Other analog nucleic acids include those with positive backbones (Denpcy, et al. (1995) Proc. Nat'l Acad. Sci. USA 92:6097-101); non-ionic backbones (U.S. Pat. Nos. 5,386,023, 5,637,684, 5,602,240, 5,216,141, and 4,469,863; Kiedrowski, et al. (1991) Angew. Chem. Intl. Ed. English 30:423-426; Letsinger, et al. (1988) J. Am. Chem. Soc. 110:4470-471; Jung, et al. (1994) Nucleoside and Nucleotide 13:1597-xxx; Chapters 2 and 3 in Sanghvi and Cook (eds. 1994) Carbohydrate Modifications in Antisense Research ACS Symposium Series 580; Mesmaeker, et al. (1994) Bioorganic and Medicinal Chem. Lett. 4:395-398; Jeffs, et al. (1994) J. Biomolecular NMR 34:17; and Horn, et al. (1996) Tetrahedron Lett. 37:743-xxx) and non-ribose backbones, including those described in U.S. Pat. Nos. 5,235,033 and 5,034,506, and Chapters 6 and 7 in Sanghvi and Cook (eds. 1994) Carbohydrate Modifications in Antisense Research ACS Symposium Series 580. Nucleic acids containing one or more carbocyclic sugars are also included within one definition of nucleic acids. See Jenkins, et al. (1995) Chem. Soc. Rev. pp 169-176. Several nucleic acid analogs are described in Rawls (page 35, Jun. 2, 1997) C&E News. All of these references are hereby expressly incorporated by reference.

Particularly preferred are peptide nucleic acids (PNA) which includes peptide nucleic acid analogs. These backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tm) for mismatched versus perfectly matched base pairs. DNA and RNA typically exhibit a 2-4° C. drop in Tm for an internal mismatch. With the non-ionic PNA backbone, the drop is closer to 7-9° C. Similarly, due to their non-ionic nature, hybridization of the bases attached to these backbones is relatively insensitive to salt concentration. In addition, PNAs are not degraded by cellular enzymes, and thus can be more stable.

The nucleic acids may be single stranded or double stranded, as specified, or contain portions of both double stranded or single stranded sequence. As will be appreciated by those in the art, the depiction of a single strand also defines the sequence of the complementary strand; thus the sequences described herein also provide the complement of the sequence. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc. “Transcript” typically refers to a naturally occurring RNA, e.g., a pre-mRNA, hnRNA, or mRNA. As used herein, the term “nucleoside” includes nucleotides and nucleoside and nucleotide analogs, and modified nucleosides such as amino modified nucleosides. In addition, “nucleoside” includes non-naturally occurring analog structures. Thus, e.g., the individual units of a peptide nucleic acid, each containing a base, are referred to herein as a nucleoside.

A “label” or a “detectable moiety” is a composition detectable by spectroscopic, photochemical, biochemical, immunochemical, chemical, physiological, or other physical means. For example, useful labels include 32_{P}, fluorescent dyes, electron-dense reagents, enzymes (e.g., as commonly used in an ELISA), biotin, digoxigenin, or haptens and proteins or other entities which can be made detectable, e.g., by incorporating a radiolabel into the peptide or used to detect antibodies specifically reactive with the peptide. The labels may be incorporated into the Hepatitis C infection nucleic acids, proteins, and antibodies. Many methods known for conjugating the antibody to the label may be employed. See, e.g., Hunter, et al. (1962) Nature 144:945; David, et al. (1974) Biochemistry 13:1014-1021; Pain, et al. (1981) J. Immunol. Meth. 40:219-230; and Nygren (1982) J. Histochem. and Cytochem. 30:407-412.

An “effector” or “effector moiety” or “effector component” is a molecule that is bound (or linked, or conjugated), either covalently, through a linker or a chemical bond, or noncovalently, through ionic, van der Waals, electrostatic, or hydrogen bonds, to an antibody. The “effector” can be a variety of molecules including, e.g., detection moieties including radioactive compounds, fluorescent compounds, an enzyme or substrate, tags such as epitope tags, a toxin; activatable moieties, a chemotherapeutic agent; a lipase; an antibiotic; or a radioisotope emitting “hard” e.g., beta radiation. The effectors may be fusion proteins, or even natural components of antibodies, e.g., Ig constant effector sequences.

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe. Alternatively, method using high affinity interactions may achieve the same results where one of a pair of binding partners binds to the other, e.g., biotin, streptavidin.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (e.g., A, G, C, or T) or modified bases (7-deazaguanosine, inosine, etc.). In addition, the bases in a probe may be joined by a linkage other than a phosphodiester bond, so long as it does not functionally interfere with hybridization. Thus, e.g., probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence. Diagnosis or prognosis may be based at the genomic level, or at the level of RNA or protein expression.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed or not expressed at all. By the term “recombinant nucleic acid” herein is meant nucleic acid, originally formed in vitro, in general, by the manipulation of nucleic acid, e.g., using polymerases and endonucleases, in a form not normally found in nature. In this manner, operably linkage of different sequences is achieved. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, e.g., using the in vivo cellular machinery of the host cell rather than in vitro manipulations; however, such nucleic acids, once produced recombinantly, although subsequently replicated non-recombinantly, are still considered recombinant for the purposes of the invention. Similarly, a “recombinant protein” is a protein made using recombinant techniques, e.g., through the expression of a recombinant nucleic acid as depicted above.

The term “heterologous” when used with reference to portions of a nucleic acid indicates that the nucleic acid comprises two or more subsequences that are not normally found in the same relationship to each other in nature. For instance, the nucleic acid is typically recombinantly produced, having two or more sequences, e.g., from unrelated genes arranged to make a new functional nucleic acid, e.g., a promoter from one source and a coding region from another source. Similarly, a heterologous protein will often refer to two or more subsequences that are not found in the same relationship to each other in nature (e.g., a fusion protein).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in “Overview of principles of hybridization and the strategy of nucleic acid assays” in Tijssen (1993) Hybridization with Nucleic Probes (Laboratory Techniques in Biochemistry and Molecular Biology) (vol. 24) Elsevier. Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C. For PCR, a temperature of about 36° C. is typical for low stringency amplification, although annealing temperatures may vary between about 32-48° C. depending on primer length. For high stringency PCR amplification, a temperature of about 62° C. is typical, although high stringency annealing temperatures can range from about 50-65° C., depending on the primer length and specificity. Typical cycle conditions for both high and low stringency amplifications include a denaturation phase of 90-95° C. for 30-120 sec, an annealing phase lasting 30-120 sec, and an extension phase of about 72° C. for 1-2 min. Protocols and guidelines for low and high stringency amplification reactions are provided, e.g., in Innis, et al. (1990) PCR Protocols, A Guide to Methods and Applications, Academic Press, NY.

Nucleic acids that do not hybridize to each other under stringent conditions are still substantially identical if the polypeptides which they encode are substantially identical. This occurs, e.g., when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous references, e.g., Ausubel, et al. (eds. 1991 and supplements) Current Protocols in Molecular Biology Lippincott.

The phrase “functional effects” in the context of assays for testing compounds that modulate activity of a Hepatitis C infection protein includes the determination of a parameter that is indirectly or directly under the influence of the Hepatitis C infection protein or nucleic acid, e.g., a functional, physical, or chemical effect, such as the ability to decrease Hepatitis C infection or its secondary consequences. It includes ligand binding activity; “Functional effects” include in vitro, in vivo, and ex vivo activities.

By “determining the functional effect” is meant assaying for a compound that modifies, e.g., increases or decreases, a parameter that is indirectly or directly under the influence of a Hepatitis C infection protein sequence, e.g., functional, enzymatic, physical, physiological, or chemical effects. Such functional effects can be measured by any means known to those skilled in the art, e.g., changes in spectroscopic characteristics (e.g., fluorescence, absorbance, refractive index), hydrodynamic (e.g., shape), chromatographic, or solubility properties for the protein, measuring inducible markers or transcriptional activation of the Hepatitis C infection protein; measuring binding activity or binding assays, e.g., binding to antibodies or other ligands, and measuring cellular proliferation. Determination of the functional effect of a compound on Hepatitis C infection and its secondary consequences can also be performed using assays known to those of skill in the art such as an in vitro assays. The functional effects can be evaluated by many means known to those skilled in the art, e.g., measurement of changes in RNA or protein levels for Hepatitis C infection-associated sequences, measurement of RNA stability, identification of downstream or reporter gene expression (CAT, luciferase, β-gal, GFP, and the like), e.g., via chemiluminescence, fluorescence, calorimetric reactions, antibody binding, inducible markers, and ligand binding assays.

“Inhibitors”, “activators”, and “modulators” of Hepatitis C infection polynucleotide and polypeptide sequences are used to refer to activating, inhibitory, or modulating molecules or compounds identified using in vitro and in vivo assays of polynucleotide and polypeptide sequences associated with Hepatitis C infection and/or its secondary consequences. Inhibitors are compounds that, e.g., bind to, partially or totally block activity, decrease, prevent, delay activation, inactivate, desensitize, or down regulate the activity or expression of Hepatitis C infection proteins, e.g., antagonists. Antisense nucleic acids may seem to inhibit expression and subsequent function of the protein. “Activators” are compounds that increase, open, activate, facilitate, enhance activation, sensitize, agonize, or up regulate Hepatitis C infection protein activity. Inhibitors, activators, or modulators also include genetically modified versions of Hepatitis C infection proteins, e.g., versions with altered activity, as well as naturally occurring and synthetic ligands, antagonists, agonists, antibodies, small chemical molecules, and the like. Such assays for inhibitors and activators include, e.g., expressing the Hepatitis C infection protein in vitro, in cells, or cell membranes, applying putative modulator compounds, and then determining the functional effects on activity, as described above. Activators and inhibitors of Hepatitis C infection and its secondary consequences can also be identified by incubating Hepatitis C infected cells and tissues or cells and tissues secondarily affected by Hepatitis C infection with the test compound and determining increases or decreases in the expression of 1 or more Hepatitis C infection proteins, e.g., 1, 2, 3, 4, 5, 10, 15, 20, 25, 30, 40, 50, or more Hepatitis C infection proteins, such as Hepatitis C infection proteins encoded by the sequences set out in Tables 1A-15.

Samples or assays comprising Hepatitis C infection proteins that are treated with a potential activator, inhibitor, or modulator are compared to control samples without the inhibitor, activator, or modulator to examine the extent of inhibition. Control samples (untreated with inhibitors) are assigned a relative protein activity value of 100%. Inhibition of a polypeptide is achieved when the activity value relative to the control is about 80%, preferably about 50%, more preferably about 25-0%. Activation of a Hepatitis C infection polypeptide is achieved when the activity value relative to the control (untreated with activators) is about 110%, more preferably 150%, more preferably 200-500% (e.g., about two to five fold higher relative to the control), more preferably about 1000-3000% higher. However, sometimes selectivity or specificity of response in the correct organs may be significant consideration relative to absolute change.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively. Typically, the antigen-binding region of an antibody or its functional equivalent will be most critical in specificity and affinity of binding. See Paul (ed. 1999) Fundamental Immunology (4th ed.) Raven.

An exemplary imrnunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, e.g., pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region. See Paul (ed. 1999) Fundamental Immunology (4th ed.) Raven. While various antibody fragments are defined in terms of the digestion of an intact antibody, it will be appreciated that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty, et al. (1990) Nature 348:552-554).

For preparation of antibodies, e.g., recombinant, monoclonal, or polyclonal antibodies, many techniques can be used. See, e.g., Kohler and Milstein (1975) Nature 256:495-497; Kozbor, et al. (1983) Immunology Today 4:72; Cole, et al. (1985) pp. 77-96 in Reisfeld and Sell (1985) Monoclonal Antibodies and Cancer Therapy Liss; Coligan (1991) Current Protocols in Immunology Lippincott; Harlow and Lane (1988) Antibodies: A Laboratory Manual CSH Press; and Goding (1986) Monoclonal Antibodies: Principles and Practice (2d ed.) Academic Press. Techniques for the production of single chain antibodies (U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens. See, e.g., McCafferty, et al. (1990) Nature 348:552-554; Marks, et al. (1992) Biotechnology 10:779-783.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

### Identification of Hepatitis C Infection-Associated Sequences

In one aspect, the expression levels of genes are determined in different patient samples for which diagnosis information is desired, to provide expression profiles. An expression profile of a particular sample is essentially a “fingerprint” of the state of the sample; while two states may have any particular gene similarly expressed, the evaluation of a number of genes simultaneously allows the generation of a gene expression profile that is characteristic of the state of the cell. That is, normal tissue may be distinguished from Hepatitis C infected tissue or cells or tissues and cells affected secondarily by Hepatitis C infection, by comparison with tissue or cell samples from uninfected individuals. By comparison of expression profiles derived from infected and uninfected individuals information regarding which genes are important (including both up- and down-regulation of genes) in each of these states is obtained.

The identification of sequences that are differentially expressed in Hepatitis C infected and non-infected individuals allows the use of this information in a number of ways. For example, a particular treatment regime may be evaluated: does a drug act to down-regulate Hepatitis C infection and/or its secondary effects, in a particular patient. Similarly, diagnosis and treatment outcomes may be done or confirmed by comparing patient samples with the known expression profiles. Furthermore, these gene expression profiles (or individual genes) allow screening of drug candidates with an eye to mimicking or altering a particular expression profile; e.g., screening can be done for drugs that suppress certain aspects of the Hepatitis C infected patient's expression profile. This may be done by making biochips comprising sets of the important Hepatitis C infection genes, which can then be used in these screens. These methods can also be done on the protein basis; that is, protein expression levels of the Hepatitis C infection proteins can be evaluated for diagnostic purposes or to screen candidate agents. In addition, the Hepatitis C infection nucleic acid sequences can be administered for gene therapy purposes, including the administration of antisense nucleic acids, or the Hepatitis C infection proteins (including antibodies and other modulators thereof) administered as therapeutic drugs.

Thus the present invention provides nucleic acid and protein sequences that are differentially expressed in Hepatitis C infected individuals, herein termed “Hepatitis C infection sequences.” Further, the invention provides means to distinguish subsets of infected individuals; e.g., those who will or will not respond to particular therapeutic treatment. As outlined below, Hepatitis C infection sequences include those that are up-regulated (e.g., expressed at a higher level) during the course of Hepatitis C infection, as well as those that are down-regulated (e.g., expressed at a lower level). In a preferred embodiment, the Hepatitis C infection sequences are from primates, e.g., humans; however, as will be appreciated by those in the art, Hepatitis C infection sequences from other organisms may be useful in animal models of disease and drug evaluation; thus, other Hepatitis C infection sequences are provided, from vertebrates, including mammals, including rodents (rats, mice, hamsters, guinea pigs, etc.), primates, farm animals (including sheep, goats, pigs, cows, horses, etc.), and pets (e.g., dogs, cats, etc.). Hepatitis C infection sequences from other organisms may be obtained using the techniques outlined below.

Hepatitis C infection sequences can include both nucleic acid and amino acid sequences. As will be appreciated by those in the art and is more fully outlined below, Hepatitis C infection nucleic acid sequences are useful in a variety of applications, including diagnostic applications, which will detect naturally occurring nucleic acids, as well as screening applications; e.g., biochips comprising nucleic acid probes or PCR microtiter plates with selected probes to the Hepatitis C infection sequences can be generated.

A Hepatitis C infection sequence can be initially identified by substantial nucleic acid and/or amino acid sequence homology to the Hepatitis C infection sequences outlined herein. Such homology can be based upon the overall nucleic acid or amino acid sequence, and is generally determined as outlined below, using either homology programs or hybridization conditions.

For identifying Hepatitis C infection-associated sequences, the screen typically includes comparing the expression of genes from different tissues, but typically liver biopsy samples, of infected versus uninfected individuals. Analysis of samples from treatment responsive and treatment non-responsive patients may also be performed. Samples obtained are applied, e.g., to biochips comprising nucleic acid probes. The samples are first microdissected, if applicable, and treated as is known in the art for the preparation of mRNA. Suitable biochips are commercially available, e.g., from Affymetrix. Gene expression profiles as described herein are generated and the data analyzed. Other means for analysis may also be performed, e.g., PCR based, protein, or antibody diagnosis.

In one embodiment, the genes showing changes in expression as between normal and disease states are compared. In a preferred embodiment, those genes identified during the screen of infected individuals, that are expressed in any significant amount in uninfected individuals are removed from the profile, although in some embodiments, this is not necessary. That is, when screening for drugs, it is usually preferable that the target be disease specific, to minimize possible side effects.

In a preferred embodiment, Hepatitis C infection sequences are those that are up-regulated during Hepatitis C infection; that is, the expression of these genes is higher in the tissues of Hepatitis C infected individuals as compared to the tissues of uninfected individuals. “Up-regulation” as used herein often means at least about a two-fold change, preferably at least about a three fold change, with at least about five-fold or higher being preferred. Unigene cluster identification numbers and accession numbers herein are for the GenBank sequence database and the sequences of the accession numbers are hereby expressly incorporated by reference. GenBank is known in the art, see, e.g., Benson, et al. (1998) Nuc. Acids Res. 26:1-7; and http://www.ncbi.nlm.nih.gov/. Sequences are also available in other databases, e.g., European Molecular Biology Laboratory (EMBL) and DNA Database of Japan (DDBJ). In some situations, the sequences may be derived from assembly of available sequences or be predicted from genomic DNA using exon prediction algorithms, such as FGENESH. See Salamov and Solovyev (2000) Genome Res. 10:516-522. In other situations, sequences have been derived from cloning and sequencing of isolated nucleic acids.

In another preferred embodiment, Hepatitis C infection sequences are those that are down-regulated in Hepatitis C infected individuals; that is, the expression of these genes is lower in tissue from individuals infected with Hepatitis C as compared to non-infected individuals. “Down-regulation” as used herein often means at least about a two-fold change, preferably at least about a three fold change, with at least about five-fold or higher being preferred.

In other embodiments, sequences which are diagnostic, or prognostic, of response to treatment are identified. In particular, markers which are diagnostic of either response or non-response to treatment are described.

Informatics

The ability to identify genes that are over or under expressed during Hepatitis C infection or treatment can additionally provide high-resolution, high-sensitivity datasets which can be used in the areas of diagnostics, therapeutics, drug development, pharmacogenetics, protein structure, biosensor development, and other related areas. For example, the expression profiles can be used in diagnostic or prognostic evaluation of patients suffering from liver conditions, particularly Hepatitis infections. Or as another example, subcellular toxicological information can be generated to better direct drug structure and activity correlation. See Anderson (Jun. 11-12, 1998) Pharmaceutical Proteomics: Targets, Mechanism, and Function, paper presented at the IBC Proteomics conference, Coronado, Calif. Subcellular toxicological information can also be utilized in a biological sensor device to predict the likely toxicological effect of chemical exposures and likely tolerable exposure thresholds (see U.S. Pat. No. 5,811,231). Similar advantages accrue from datasets relevant to other biomolecules and bioactive agents (e.g., nucleic acids, saccharides, lipids, drugs, and the like).

Thus, in another embodiment, the present invention provides a database that includes at least one set of assay data. The data contained in the database is acquired, e.g., using array analysis either singly or in a library format. The database can be in many forms in which data can be maintained and transmitted, but is preferably an electronic database. The electronic database of the invention can be maintained on any electronic device allowing for the storage of and access to the database, such as a personal computer, but is preferably distributed on a wide area network, such as the World Wide Web.

The focus of the present section on databases that include nucleic acid, and corresponding peptide, sequence data is for clarity of illustration only. It will be apparent to those of skill in the art that similar databases can be assembled for an assay data acquired using an assay of the invention.

The compositions and methods for identifying and/or quantitating the relative and/or absolute abundance of a variety of molecular and macromolecular species from a biological sample experiencing hepatitis C infection or its secondary consequences, e.g., the identification of Hepatitis C infection-associated sequences described herein, provide an abundance of information, which can be correlated with pathological conditions, drug testing, therapeutic monitoring, gene-disease causal linkages, identification of correlates of immunity and physiological status, subsetting of patients into particular treatment responsive or non-responsive groups, among others. Although the data generated from the assays of the invention is suited for manual review and analysis, in a preferred embodiment, prior data processing using high-speed computers is utilized.

An array of methods for indexing and retrieving biomolecular information is known in the art. For example, U.S. Pat. Nos. 6,023,659 and 5,966,712 disclose a relational database system for storing biomolecular sequence information in a manner that allows sequences to be catalogued and searched according to one or more protein function hierarchies. U.S. Pat. No. 5,953,727 discloses a relational database having sequence records containing information in a format that allows a collection of partial-length DNA sequences to be catalogued and searched according to association with one or more sequencing projects for obtaining full-length sequences from the collection of partial length sequences. U.S. Pat. No. 5,706,498 discloses a gene database retrieval system for making a retrieval of a gene sequence similar to a sequence data item in a gene database based on the degree of similarity between a key sequence and a target sequence. U.S. Pat. No. 5,538,897 discloses a method using mass spectroscopy fragmentation patterns of peptides to identify amino acid sequences in computer databases by comparison of predicted mass spectra with experimentally-derived mass spectra using a closeness-of-fit measure. U.S. Pat. No. 5,926,818 discloses a multi-dimensional database comprising a functionality for multi-dimensional data analysis described as on-line analytical processing (OLAP), which entails the consolidation of projected and actual data according to more than one consolidation path or dimension. U.S. Pat. No. 5,295,261 reports a hybrid database structure in which the fields of each database record are divided into two classes, navigational and informational data, with navigational fields stored in a hierarchical topological map which can be viewed as a tree structure or as the merger of two or more such tree structures. See also Mount (2001) Bioinformatics: Sequence and Genome Analysis CSH Press, NY; Durbin, et al. (eds. 1999) Biological Sequence Analysis: Probabilistic Models of Proteins and Nucleic Acids Cambridge Univ. Press; Baxevanis and Oeullette (eds. 1998) Bioinformatics: A Practical Guide to the Analysis of Genes and Proteins (2d ed.) Wiley-Liss; Rashidi and Buehler (1999) Bioinformatics: Basic Applications in Biological Science and Medicine CRC Press; Setubal, et al. (eds. 1997) Introduction to Computational Molecular Biology Brooks/Cole; Misener and Krawetz (eds. 2000) Bioinformatics: Methods and Protocols Humana Press; Higgins and Taylor (eds. 2000) Bioinformatics: Sequence, Structure, and Databanks: A Practical Approach Oxford Univ. Press; Brown (2001) Bioinformatics: A Biologist's Guide to Biocomputing and the Internet Eaton Pub.; Han and Kamber (2000) Data Mining: Concepts and Techniques Kaufmann Pub.; and Waterman (1995) Introduction to Computational Biology: Maps, Sequences, and Genomes Chap and Hall.

The present invention provides a computer database comprising a computer and software for storing in computer-retrievable form assay data records cross-tabulated, e.g., with data specifying the source of the target-containing sample from which each sequence specificity record was obtained, and perhaps patient data or response.

In an exemplary embodiment, at least one of the sources of target-containing sample is from a control tissue sample known to be free of pathological disorders. In a variation, at least one of the sources is a known pathological tissue specimen. In another variation, the assay records cross-tabulate one or more of the following parameters for each target species in a sample: (1) a unique identification code, which can include, e.g., a target molecular structure and/or characteristic separation coordinate (e.g., electrophoretic coordinates); (2) sample source; (3) absolute and/or relative quantity of the target species present in the sample; and (4) patient history or eventual treatment outcome.

The invention also provides for the storage and retrieval of a collection of target data in a computer data storage apparatus, which can include magnetic disks, optical disks, magneto-optical disks, DRAM, SRAM, SGRAM, SDRAM, RDRAM, DDR RAM, magnetic bubble memory devices, and other data storage devices, including CPU registers and on-CPU data storage arrays. Typically, the target data records are stored as a bit pattern in an array of magnetic domains on a magnetizable medium or as an array of charge states or transistor gate states, such as an array of cells in a DRAM device (e.g., each cell comprised of a transistor and a charge storage area, which may be on the transistor). In one embodiment, the invention provides such storage devices, and computer systems built therewith, comprising a bit pattern encoding a protein expression fingerprint record comprising unique identifiers for at least 10 target data records cross-tabulated with target source.

When the target is a peptide or nucleic acid, the invention preferably provides a method for identifying related peptide or nucleic acid sequences, comprising performing a computerized comparison between a peptide or nucleic acid sequence assay record stored in or retrieved from a computer storage device or database and at least one other sequence. The comparison can include a sequence analysis or comparison algorithm or computer program embodiment thereof (e.g., FASTA, TFASTA, GAP, BESTFIT) and/or the comparison may be of the relative amount of a peptide or nucleic acid sequence in a pool of sequences determined from a polypeptide or nucleic acid sample of a specimen.

The invention also preferably provides a magnetic disk, such as an IBM-compatible (DOS, Windows, Windows95/98/2000, Windows NT, OS/2) or other format (e.g., Linux, SunOS, Solaris, AIX, SCO Unix, VMS, MV, Macintosh, etc.) floppy diskette or hard (fixed, Winchester) disk drive, comprising a bit pattern encoding data from an assay of the invention in a file format suitable for retrieval and processing in a computerized sequence analysis, comparison, or relative quantitation method.

The invention also provides a network, comprising a plurality of computing devices linked via a data link, such as an Ethernet cable (coax or 10BaseT), telephone line, ISDN line, wireless network, optical fiber, or other suitable signal transmission medium, whereby at least one network device (e.g., computer, disk array, etc.) comprises a pattern of magnetic domains (e.g., magnetic disk) and/or charge domains (e.g., an array of DRAM cells) composing a bit pattern encoding data acquired from an assay of the invention.

The invention also provides a method for transmitting assay data that includes generating an electronic signal on an electronic communications device, such as a modem, ISDN terminal adapter, DSL, cable modem, ATM switch, or the like, wherein the signal includes (in native or encrypted format) a bit pattern encoding data from an assay or a database comprising a plurality of assay results obtained by the method of the invention.

In a preferred embodiment, the invention provides a computer system for comparing a query target to a database containing an array of data structures, such as an assay result obtained by the method of the invention, and ranking database targets based on the degree of identity and gap weight to the target data. A central processor is preferably initialized to load and execute the computer program for alignment and/or comparison of the assay results. Data for a query target is entered into the central processor via an I/O device. Execution of the computer program results in the central processor retrieving the assay data from the data file, which comprises a binary description of an assay result.

The target data or record and the computer program can be transferred to secondary memory, which is typically random access memory (e.g., DRAM, SRAM, SGRAM, or SDRAM). Targets are ranked according to the degree of correspondence between a selected assay characteristic (e.g., binding to a selected affinity moiety) and the same characteristic of the query target and results are output via an I/O device. For example, a central processor can be a conventional computer (e.g., Intel Pentium, PowerPC, Alpha, PA-8000, SPARC, MIPS 4400, MIPS 10000, VAX, etc.); a program can be a commercial or public domain molecular biology software package (e.g., UWGCG Sequence Analysis Software, Darwin); a data file can be an optical or magnetic disk, a data server, a memory device (e.g., DRAM, SRAM, SGRAM, SDRAM, EPROM, bubble memory, flash memory, etc.); an I/O device can be a terminal comprising a video display and a keyboard, a modem, an ISDN terminal adapter, an Ethernet port, a punched card reader, a magnetic strip reader, or other suitable I/O device.

The invention also preferably provides the use of a computer system, such as that described above, which comprises: (1) a computer; (2) a stored bit pattern encoding a collection of peptide sequence specificity records obtained by the methods of the invention, which may be stored in the computer; (3) a comparison target, such as a query target; and (4) a program for alignment and comparison, typically with rank-ordering of comparison results on the basis of computed similarity values.

Characteristics of Hepatitis C Infection-Associated Proteins

Hepatitis C infection proteins of the present invention may be classified as secreted proteins, transmembrane proteins, or intracellular proteins. In one embodiment, the Hepatitis C infection protein is an intracellular protein. Intracellular proteins may be found in the cytoplasm and/or in the nucleus. Intracellular proteins are involved in all aspects of cellular function and replication (including, e.g., signaling pathways); aberrant expression of such proteins often results in unregulated or disregulated cellular processes. See, e.g., Alberts, et al. (eds. 1994) Molecular Biology of the Cell (3d ed.) Garland. For example, many intracellular proteins have enzymatic activity such as protein kinase activity, protein phosphatase activity, protease activity, nucleotide cyclase activity, polymerase activity, and the like. Intracellular proteins also serve as docking proteins that are involved in organizing complexes of proteins, or targeting proteins to various subcellular localizations, and are involved in maintaining the structural integrity of organelles. In particular, many of the genes identified in the analysis of the present invention may result from virus infection or related physiology, e.g., immunological responses, etc.

An increasingly appreciated concept in characterizing proteins is the presence in the proteins of one or more motifs for which defined functions have been attributed. In addition to the highly conserved sequences found in the enzymatic domain of proteins, highly conserved sequences have been identified in proteins that are involved in protein-protein interaction. For example, Src-homology-2 (SH2) domains bind tyrosine-phosphorylated targets in a sequence dependent manner. PTB domains, which are distinct from SH2 domains, also bind tyrosine phosphorylated targets. SH3 domains bind to proline-rich targets. In addition, PH domains, tetratricopeptide repeats, and WD domains to name only a few, have been shown to mediate protein-protein interactions. Some of these may also be involved in binding to phospholipids or other second messengers. As will be appreciated by one of ordinary skill in the art, these motifs can be identified on the basis of primary sequence; thus, an analysis of the sequence of proteins may provide insight into both the enzymatic potential of the molecule and/or molecules with which the protein may associate. One useful database is Pfam (protein families), which is a large collection of multiple sequence alignments and hidden Markov models covering many common protein domains. Versions are available via the internet from Washington University in St. Louis, the Sanger Center in England, and the Karolinska Institute in Sweden. See, e.g., Bateman, et al. (2000) Nuc. Acids Res. 28:263-266; Sonnhammer, et al. (1997) Proteins 28:405-420; Bateman, et al. (1999) Nuc. Acids Res. 27:260-262; and Sonnhammer, et al. (1998) Nuc. Acids Res. 26:320-322.

In another embodiment, the Hepatitis C infection sequences are transmembrane proteins. Transmembrane proteins are molecules that span a phospholipid bilayer of a cell. They may have an intracellular domain, an extracellular domain, or both. The intracellular domains of such proteins may have a number of functions including those already described for intracellular proteins. For example, the intracellular domain may have enzymatic activity and/or may serve as a binding site for additional proteins. Frequently the intracellular domain of transmembrane proteins serves both roles. For example certain receptor tyrosine kinases have both protein kinase activity and SH2 domains. In addition, autophosphorylation of tyrosines on the receptor molecule itself, creates binding sites for additional SH2 domain containing proteins.

Transmembrane proteins may contain from one to many transmembrane domains. For example, receptor tyrosine kinases, certain cytokine receptors, receptor guanylyl cyclases, and receptor serine/threonine protein kinases contain a single transmembrane domain. However, various other proteins including channels and adenylyl cyclases contain numerous transmembrane domains. Many important cell surface receptors such as G protein coupled receptors (GPCRs) are classified as “seven transmembrane domain” proteins, as they contain 7 membrane spanning regions. Characteristics of transmembrane domains include approximately 20 consecutive hydrophobic amino acids that may be followed by charged amino acids. Therefore, upon analysis of the amino acid sequence of a particular protein, the localization and number of transmembrane domains within the protein may be predicted (see, e.g., PSORT web site http://psort.nibb.ac.jp/). Important transmembrane protein receptors include, but are not limited to, the insulin receptor, insulin-like growth factor receptor, human growth hormone receptor, glucose transporters, transferrin receptor, epidermal growth factor receptor, low density lipoprotein receptor, epidermal growth factor receptor, leptin receptor, interleukin receptors, e.g., IL-1 receptor, IL-2 receptor, and other identified cytokine receptors, and chemokine receptors.

The extracellular domains of transmembrane proteins are diverse; however, conserved motifs are found repeatedly among various extracellular domains. Conserved structure and/or functions have been ascribed to different extracellular motifs. Many extracellular domains are involved in binding to other molecules. In one aspect, extracellular domains are found on receptors. Factors that bind the receptor domain include circulating ligands, which may be peptides, proteins, or small molecules such as adenosine and the like. For example, growth factors such as EGF, FGF, and PDGF are circulating growth factors that bind to their cognate receptors to initiate a variety of cellular responses. Other factors include cytokines, mitogenic factors, neurotrophic factors, and the like. Extracellular domains also bind to cell-associated molecules. In this respect, they mediate cell-cell interactions. Cell-associated ligands can be tethered to the cell, e.g., via a glycosylphosphatidylinositol (GPI) anchor, or may themselves be transmembrane proteins. Extracellular domains also associate with the extracellular matrix and contribute to the maintenance of the cell structure.

Transmembrane proteins that are associated with Hepatitis C infection are particularly preferred in the present invention as they are readily accessible targets for immunotherapeutics, as are described herein. In addition, as outlined below, transmembrane proteins can be also useful in imaging modalities. Antibodies may be used to label such readily accessible proteins in situ. Alternatively, antibodies can also label intracellular proteins, in which case samples are typically permeablized to provide access to intracellular proteins. Diagnosis may be of biopsy samples isolated from the individual, including serum or liver samples.

It will also be appreciated by those in the art that a transmembrane protein can be made soluble by removing transmembrane sequences, e.g., through recombinant methods. Furthermore, transmembrane proteins that have been made soluble can be made to be secreted through recombinant means by adding an appropriate signal sequence. Alternatively, normal or pathological processes may result in the release of membrane or intracellular proteins into the serum, e.g., by proteolytic processing to release portions of a protein into a body fluid.

In another embodiment, the Hepatitis C infection proteins are secreted proteins; the secretion of which can be either constitutive or regulated. These proteins have a signal peptide or signal sequence that targets the molecule to the secretory pathway. Secreted proteins are involved in numerous physiological events; often by virtue of their circulating nature, they serve to transmit signals to various other cell types. The secreted protein may function in an autocrine manner (acting on the cell that secreted the factor), a paracrine manner (acting on cells in close proximity to the cell that secreted the factor), an endocrine manner (acting on cells at a distance, e.g., secretion into the blood stream), or exocrine (secretion, e.g., through a duct or to adjacent epithelial surface as sweat glands, sebaceous glands, pancreatic ducts, lacrimal glands, mammary glands, wax producing glands of the ear, etc.). Thus secreted molecules find use in modulating or altering numerous aspects of physiology. Hepatitis C infection proteins that are secreted proteins are particularly preferred in the present invention as they serve as good therapeutic targets and also as diagnostic markers, e.g., for blood, plasma, serum, or stool tests. Diagnosis may be direct for the protein, or of a response to the protein, e.g., presence of antibodies generated to the protein. Those which are enzymes may be antibody or small molecule targets. Others may be useful as vaccine targets, e.g., via CTL mechanisms.

Use of Hepatitis C Infection Nucleic Acids

As described above, a Hepatitis C infection sequence is initially identified by substantial nucleic acid and/or amino acid sequence homology or linkage to the Hepatitis C infection sequences outlined herein. Such homology can be based upon the overall nucleic acid or amino acid sequence, and is generally determined as outlined below, using either homology programs or hybridization conditions. Typically, linked sequences on a mRNA are found on the same molecule.

The Hepatitis C infection nucleic acid sequences of the invention, e.g., the sequences in Tables 1A-15, can be fragments of larger genes, e.g., they are nucleic acid segments. “Genes” in this context includes coding regions, non-coding regions, and mixtures of coding and non-coding regions. Accordingly, as will be appreciated by those in the art, using the sequences provided herein, extended sequences, in either direction, of the Hepatitis C infection genes can be obtained, using techniques well known in the art for cloning either longer sequences or the full length sequences; see Ausubel, et al., supra. Much can be done by informatics and many sequences can be clustered to include multiple sequences corresponding to a single gene, e.g., systems such as UniGene (see, http://www.ncbi.nlm.nih.gov/UniGene/).

Once the Hepatitis C infection nucleic acid is identified, it can be cloned and, if necessary, its constituent parts recombined to form the entire Hepatitis C infection nucleic acid coding regions or the entire mRNA sequence. Once isolated from its natural source, e.g., contained within a plasmid or other vector or excised therefrom as a linear nucleic acid segment, the recombinant Hepatitis C infection nucleic acid can be further used as a probe to identify and isolate other Hepatitis C infection nucleic acids, e.g., extended coding regions. It can also be used as a “precursor” nucleic acid to make modified or variant Hepatitis C infection nucleic acids and proteins.

The Hepatitis C infection nucleic acids of the present invention are used in several ways. In a first embodiment, nucleic acid probes to the Hepatitis C infection nucleic acids are made and attached to biochips to be used in screening and diagnostic methods, as outlined below, or for administration, e.g., for gene therapy, vaccine, and/or antisense applications. Alternatively, the Hepatitis C infection nucleic acids that include coding regions of Hepatitis C infection proteins can be put into expression vectors for the expression of Hepatitis C infection proteins, again for screening purposes, for administration to a patient, to generate a vaccine, or to generate antibodies.

In a preferred embodiment, nucleic acid probes to Hepatitis C infection nucleic acids (both the nucleic acid sequences outlined in the figures and/or the complements thereof) are made. The nucleic acid probes attached to the biochip are designed to be substantially complementary to the Hepatitis C infection nucleic acids, e.g., the target sequence (either the target sequence of the sample or to other probe sequences, e.g., in sandwich assays), such that hybridization of the target sequence and the probes of the present invention occurs. As outlined below, this complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions, particularly high stringency conditions, as outlined herein. PCR technologies may also be applicable.

A nucleic acid probe is generally single stranded but can be partially single and partially double stranded. The strandedness of the probe is dictated by the structure, composition, and properties of the target sequence. In general, the nucleic acid probes range from about 8-100 bases long, with from about 10-80 bases being preferred, and from about 30-50 bases being particularly preferred. That is, generally whole genes are not used. In some embodiments, much longer nucleic acids can be used, up to hundreds of bases.

In a preferred embodiment, more than one probe per sequence is used, with either overlapping probes or probes to different sections of the target being used. That is, two, three, four or more probes, with three being preferred, are used to build in a redundancy for a particular target. The probes can be overlapping (e.g., have some sequence in common), or separate. In some cases, PCR primers may be used to amplify signal for higher sensitivity.

Nucleic acids can be attached or immobilized to a solid support in a wide variety of ways. By “immobilized” and grammatical equivalents herein is meant the association or binding between the nucleic acid probe and a solid support is sufficient to be Tables 1A-15 under the conditions of binding, washing, analysis, and removal as outlined below. The binding can typically be covalent or non-covalent. By “non-covalent binding” and grammatical equivalents herein is meant one or more of electrostatic, hydrophilic, and hydrophobic interactions. Included in non-covalent binding is the covalent attachment of a molecule, such as, streptavidin to the support and the non-covalent binding of the biotinylated probe to the streptavidin. By “covalent binding” and grammatical equivalents herein is meant that the two moieties, the solid support and the probe, are attached by at least one bond, including sigma bonds, pi bonds, and coordination bonds. Covalent bonds can be formed directly between the probe and the solid support or can be formed by a cross linker or by inclusion of a specific reactive group on either the solid support or the probe or both molecules. Immobilization may also involve a combination of covalent and non-covalent interactions.

In general, the probes are attached to the biochip in a wide variety of ways, as will be appreciated by those in the art. As described herein, the nucleic acids can either be synthesized first, with subsequent attachment to the biochip, or can be directly synthesized on the biochip.

The biochip comprises a suitable solid substrate. By “substrate” or “solid support” or other grammatical equivalents herein is meant a material that can be modified to contain discrete individual sites appropriate for the attachment or association of the nucleic acid probes and is amenable to at least one detection method. As will be appreciated by those in the art, the number of possible substrates is very large, and include, but are not limited to, glass and modified or functionalized glass, plastics (including acrylics, polystyrene, and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses, plastics, etc. In general, the substrates allow optical detection and preferably do not appreciably fluoresce. See WO 00/55627.

Generally the substrate is planar, although as will be appreciated by those in the art, other configurations of substrates may be used as well. For example, the probes may be placed on the inside surface of a tube, for flow-through sample analysis to minimize sample volume. Similarly, the substrate may be flexible, such as a flexible foam, including closed cell foams made of particular plastics.

In a preferred embodiment, the surface of the biochip and the probe may be derivatized with chemical functional groups for subsequent attachment of the two. Thus, e.g., the biochip is derivatized with a chemical functional group including, but not limited to, amino groups, carboxy groups, oxo groups, and thiol groups, with amino groups being particularly preferred. Using these functional groups, the probes can be attached using functional groups on the probes. For example, nucleic acids containing amino groups can be attached to surfaces comprising amino groups, e.g., using available linkers, homo-or hetero-bifunctional linkers as are well known (see 1994 Pierce Chemical Company catalog, technical section on cross-linkers, pages 155-200). In addition, in some cases, additional linkers, such as alkyl groups (including substituted and heteroalkyl groups) may be used.

In this embodiment, oligonucleotides are synthesized as is known in the art, and then attached to the surface of the solid support. Either the 5′ or 3′ terminus may be attached to the solid support, or attachment may be via an internal nucleoside. In another embodiment, the immobilization to the solid support may be very strong, yet non-covalent. For example, biotinylated oligonucleotides can be made, which bind to surfaces covalently coated with streptavidin, resulting in attachment.

In another embodiment, the immobilization to the solid support may be very strong, yet non-covalent. For example, biotinylated oligonucleotides can be made, which bind to surfaces covalently coated with streptavidin, resulting in attachment.

Alternatively, the oligonucleotides may be synthesized on the surface, as is known in the art. For example, photoactivation techniques utilizing photopolymerization compounds and techniques are used. In a preferred embodiment, the nucleic acids can be synthesized in situ, using well known photolithographic techniques, such as those described in WO 95/25116; WO 95/35505; U.S. Pat. Nos. 5,700,637 and 5,445,934; and references cited within, all of which are expressly incorporated by reference; these methods of attachment form the basis of the Affymetrix GeneChip™ technology. Other oligonucleotide synthetic techniques may be applied.

Often, amplification-based assays are performed to measure the expression level of Hepatitis C infection-associated sequences. These assays are typically performed in conjunction with reverse transcription. In such assays, a Hepatitis C infection-associated nucleic acid sequence acts as a template in an amplification reaction (e.g., Polymerase Chain Reaction, or PCR). In a quantitative amplification, the amount of amplification product will be proportional to the amount of template in the original sample. Comparison to appropriate controls provides a measure of the amount of Hepatitis C infection-associated RNA. Methods of quantitative amplification are well known to those of skill in the art. Detailed protocols for quantitative PCR are provided, e.g., in Innis, et al. (1990) PCR Protocols: A Guide to Methods and Applications Academic Press.

In some embodiments, a TaqMan based assay is used to measure expression. TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, e.g., AmpliTaq, results in the cleavage of the TaqMan probe. This cleavage separates the 5′ fluorescent dye and the 3′ quenching agent, thereby resulting in an increase in fluorescence as a function of amplification (see, e.g., literature provided by Perkin-Elmer, e.g., www2.perkin-elmer.com).

Other suitable amplification methods include, but are not limited to, ligase chain reaction (LCR) (see Wu and Wallace (1989) Genomics 4:560-569, Landegren, et al. (1988) Science 241:1077-1080, and Barringer, et al. (1990) Gene 89:117-122), transcription amplification (Kwoh, et al. (1989) Proc. Nat'l Acad. Sci. USA 86:1173-1177), self-sustained sequence replication (Guatelli, et al. (1990) Proc. Nat'l Acad. Sci. USA 87:1874-1878), dot PCR, linker adapter PCR, etc.

Expression of Hepatitis C Infection Proteins from Nucleic Acids

In a preferred embodiment, Hepatitis C infection nucleic acids, e.g., encoding Hepatitis C infection proteins are used to make a variety of expression vectors to express Hepatitis C infection proteins which can then be used in screening assays, as described below. Expression vectors and recombinant DNA technology are well known (see, e.g., Ausubel, supra, and Fernandez and Hoeffler (eds. 1999) Gene Expression Systems Academic Press) and are used to express proteins. The expression vectors may be self-replicating extrachromosomal vectors or vectors which integrate into a host genome. Generally, these expression vectors include transcriptional and translational regulatory nucleic acid operably linked to the nucleic acid encoding the Hepatitis C infection protein. The term “control sequences” refers to DNA sequences used for the expression of an operably linked coding sequence in a particular host organism. Control sequences that are suitable for prokaryotes, e.g., include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is typically accomplished by ligation at convenient restriction sites. If such sites do not exist, synthetic oligonucleotide adaptors or linkers are used in accordance with conventional practice. Transcriptional and translational regulatory nucleic acid will generally be appropriate to the host cell used to express the Hepatitis C infection protein. Numerous types of appropriate expression vectors, and suitable regulatory sequences are known in the art for a variety of host cells.

In general, transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences.

Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition, an expression vector may comprise additional elements. For example, the expression vector may have two replication systems, thus allowing it to be maintained in two organisms, e.g., in mammalian or insect cells for expression and in a prokaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are available. See, e.g., Fernandez and Hoeffler, supra; and Kitamura, et al. (1995) Proc. Nat'l Acad. Sci. USA 92:9146-9150.

In addition, in a preferred embodiment, the expression vector contains a selectable marker gene to allow the selection of transformed host cells. Selection genes are well known in the art and will vary with the host cell used.

The Hepatitis C infection proteins of the present invention may be produced by culturing a host cell transformed with an expression vector containing nucleic acid encoding a Hepatitis C infection protein, under the appropriate conditions to induce or cause expression of the Hepatitis C infection protein. Conditions appropriate for Hepatitis C infection protein expression will vary with the choice of the expression vector and the host cell, and will be easily ascertained by one skilled in the art through routine experimentation or optimization. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield. Alternatively, cells may be identified which naturally express relevant genes at high levels.

Appropriate host cells include yeast, bacteria, archaebacteria, fungi, and insect and animal cells, including mammalian cells. Of particular interest are Saccharomyces cerevisiae and other yeasts, E. coli, Bacillus subtilis, Sf9 cells, C 129 cells, 293 cells, Neurospora, BHK, CHO, COS, HeLa cells, HUVEC (human umbilical vein endothelial cells), THP1 cells (a macrophage cell line), and various other human cells and cell lines.

In a preferred embodiment, the Hepatitis C infection proteins are expressed in mammalian cells. Mammalian expression systems are also known in the art, and include retroviral and adenoviral systems. One expression vector system is a retroviral vector system such as is generally described in PCT/US97/01019 and PCT/US97/01048, both of which are hereby expressly incorporated by reference. Of particular use as mammalian promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter (see, e.g., Fernandez and Hoeffler, supra). Typically, transcription termination and polyadenylation sequences recognized by mammalian cells are regulatory regions located 3′ to the translation stop codon and thus, together with the promoter elements, flank the coding sequence. Examples of transcription terminator and polyadenylation signals include those derived form SV40.

Methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In a preferred embodiment, Hepatitis C infection proteins are expressed in bacterial systems. Promoters from bacteriophage may also be used. In addition, synthetic promoters and hybrid promoters are also useful; e.g., the tac promoter is a hybrid of the trp and lac promoter sequences. Furthermore, a bacterial promoter can include naturally occurring promoters of non-bacterial origin that have the ability to bind bacterial RNA polymerase and initiate transcription. In addition to a functioning promoter sequence, an efficient ribosome binding site is desirable. The expression vector may also include a signal peptide sequence that provides for secretion of the Hepatitis C infection protein in bacteria. The protein is either secreted into the growth media (gram-positive bacteria) or into the periplasmic space, located between the inner and outer membrane of the cell (gram-negative bacteria). The bacterial expression vector may also include a selectable marker gene to allow for the selection of bacterial strains that have been transformed. Suitable selection genes include genes which render the bacteria resistant to drugs, e.g., ampicillin, chloramphenicol, erythromycin, kanamycin, neomycin, and tetracycline. Selectable markers also include biosynthetic genes, such as those in the histidine, tryptophan, and leucine biosynthetic pathways. These components are assembled into expression vectors. Expression vectors for bacteria are well known in the art, and include vectors for Bacillus subtilis, E. coli, Streptococcus cremoris, and Streptococcus lividans, among others (e.g., Fernandez and Hoeffler, supra). The bacterial expression vectors are transformed into bacterial host cells using techniques such as calcium chloride treatment, electroporation, and others.

In one embodiment, Hepatitis C infection proteins are produced in insect cells, e.g., expression vectors for the transformation of insect cells, and, in particular, baculovirus-based expression vectors.

In a preferred embodiment, Hepatitis C infection protein is produced in yeast cells. Yeast expression systems include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica.

The Hepatitis C infection protein may also be made as a fusion protein, e.g., for the creation of monoclonal antibodies, if the desired epitope is small, the Hepatitis C infection protein may be fused to a carrier protein to form an immunogen. Alternatively, the Hepatitis C infection protein may be made as a fusion protein to increase expression, or for other reasons. For example, when the Hepatitis C infection protein is a Hepatitis C infection peptide, the nucleic acid encoding the peptide may be linked to other nucleic acid for expression purposes. Fusion with detection epitope tags can be made, e.g., with FLAG, His6, myc, HA, etc.

In a preferred embodiment, the Hepatitis C infection protein is purified or isolated after expression. Hepatitis C infection proteins may be isolated or purified in a variety of ways depending on what other components are present in the sample. Standard purification methods include electrophoretic, molecular, immunological, and chromatographic techniques, including ion exchange, hydrophobic, affinity, reverse-phase HPLC chromatography, affinity label, and chromatofocusing. For example, the Hepatitis C infection protein may be purified using a standard anti-Hepatitis C infection protein antibody column. Ultrafiltration and diafiltration techniques, in conjunction with protein concentration, are also useful. For general guidance in suitable purification techniques, see, e.g., Scopes (1993) Protein Purification Springer-Verlag, NY. The degree of purification necessary will vary depending on the use of the Hepatitis C infection protein. In some instances no purification will be necessary.

Once expressed and purified if necessary, the Hepatitis C infection proteins and nucleic acids are useful in a number of applications. They may be used as immunoselection reagents, as vaccine reagents, as screening agents, etc.

### Variants of Hepatitis C Infection Proteins

In one embodiment, the proteins expressed as a result of Hepatitis C infection are derivative or variant proteins as compared to the wild-type sequence. That is, as outlined more fully below, the derivative Hepatitis C infection peptide will often contain at least one amino acid substitution, deletion or insertion, with amino acid substitutions being particularly preferred. The amino acid substitution, insertion or deletion may occur at virtually any residue within the Hepatitis C infection peptide.

Also included within one embodiment of Hepatitis C infection proteins of the present invention are amino acid sequence variants. These variants typically fall into one or more of three classes: substitutional, insertional or deletional variants. These variants ordinarily are prepared by site specific mutagenesis of nucleotides in the DNA encoding the Hepatitis C infection protein, using cassette or PCR mutagenesis or other techniques well known in the art, to produce DNA encoding the variant, and thereafter expressing the DNA in recombinant cell culture as outlined above. However, variant Hepatitis C infection protein fragments having up to about 100-150 residues may be prepared by in vitro synthesis using established techniques. Amino acid sequence variants are characterized by the predetermined nature of the variation, a feature that sets them apart from naturally occurring allelic or interspecies variation of the Hepatitis C infection protein amino acid sequence. The variants typically exhibit the same qualitative biological activity as the naturally occurring analogue, although variants can also be selected which have modified characteristics as will be more fully outlined below.

While the site or region for introducing an amino acid sequence variation is generally predetermined, the mutation per se need not be predetermined. For example, in order to optimize the performance of a mutation at a given site, random mutagenesis may be conducted at the target codon or region and the expressed Hepatitis C infection variants screened for the optimal combination of desired activity. Techniques for making substitution mutations at predetermined sites in DNA having a known sequence are well known, e.g., M13 primer mutagenesis and PCR mutagenesis. Screening of the mutants is done using assays of Hepatitis C infection protein activities.

Amino acid substitutions are typically of single residues; insertions usually will be on the order of from about 1-20 amino acids, although considerably larger insertions may be tolerated. Deletions range from about 1-20 residues, although in some cases deletions may be much larger.

Substitutions, deletions, insertions or combinations thereof may be used to arrive at a final derivative. Generally these changes are done on a few amino acids to minimize the alteration of the molecule. However, larger changes may be tolerated in certain circumstances. When small alterations in the characteristics of the Hepatitis C infection protein are desired, substitutions are generally made in accordance with the amino acid substitution relationships provided in the definition section.

The variants typically exhibit the same qualitative biological activity and will elicit the same immune response as the naturally-occurring analog, although variants also are selected to modify the characteristics of the Hepatitis C infection proteins as needed. Alternatively, the variant may be designed such that the biological activity of the Hepatitis C infection protein is altered. For example, glycosylation sites may be altered or removed.

Substantial changes in function or immunological identity are made by selecting substitutions that are less conservative than those described above. For example, substitutions may be made which more significantly affect: the structure of the polypeptide backbone in the area of the alteration, e.g., the alpha-helical or beta-sheet structure; the charge or hydrophobicity of the molecule at the target site; or the bulk of the side chain. The substitutions which in general are expected to produce the greatest changes in the polypeptide's properties are those in which (a) a hydrophilic sidechain, e.g., serine or threonine, is substituted for (or by) a hydrophobic sidechain, e.g., leucine, isoleucine, phenylalanine, valine, or alanine; (b) a cysteine or proline is substituted for (or by) another residue; (c) a residue having an electropositive side chain, e.g., lysine, arginine, or histidine, is substituted for (or by) an electronegative side chain, e.g., glutamic or aspartic acid; (d) a residue having a bulky side chain, e.g., phenylalanine, is substituted for (or by) one not having a side chain, e.g., glycine; or (e) a proline residue is incorporated or substituted, which changes the degree of rotational freedom of the peptidyl bond.

Covalent modifications of Hepatitis C infection polypeptides are included within the scope of this invention. One type of covalent modification includes reacting targeted amino acid residues of a Hepatitis C infection polypeptide with an organic derivatizing agent that is capable of reacting with selected side chains or the N-or C-terminal residues of a Hepatitis C infection polypeptide. Derivatization with bifunctional agents is useful, e.g., for crosslinking Hepatitis C infection polypeptides to a water-insoluble support matrix or surface for use in the method for purifying anti-Hepatitis C infection polypeptide antibodies or screening assays, as is more fully described below. Commonly used crosslinking agents include, e.g., 1,1-bis(diazoacetyl)-2-phenylethane, glutaraldehyde, N-hydroxysuccinimide esters, e.g., esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-((p-azidophenyl)dithio)propioimidate.

Other modifications include deamidation of glutaminyl and asparaginyl residues to the corresponding glutamyl and aspartyl residues, respectively, hydroxylation of proline and lysine, phosphorylation of hydroxyl groups of serinyl, threonyl or tyrosyl residues, methylation of the amino groups of the lysine, arginine, and histidine side chains (e.g., pp. 79-86, Creighton (1992) Proteins: Structure and Molecular Properties Freeman), acetylation of the N-terminal amine, and amidation of a C-terminal carboxyl group.

Another type of covalent modification of the Hepatitis C infection polypeptide included within the scope of this invention comprises altering the native glycosylation pattern of the polypeptide. “Altering the native glycosylation pattern” is intended for purposes herein to mean deleting one or more carbohydrate moieties found in native sequence Hepatitis C infection polypeptide, and/or adding one or more glycosylation sites that are not present in the native sequence Hepatitis C infection polypeptide. Glycosylation patterns can be altered in many ways. Different cell types may be used to express Hepatitis C infection-associated sequences to exhibit different glycosylation patterns.

Addition of glycosylation sites to Hepatitis C infection polypeptides may also be accomplished by altering the amino acid sequence thereof. The alteration may be made, e.g., by the addition of, or substitution by, one or more serine or threonine residues to the native sequence Hepatitis C infection polypeptide (for O-linked glycosylation sites). The Hepatitis C infection amino acid sequence may optionally be altered through changes at the DNA level, particularly by mutating the DNA encoding the Hepatitis C infection polypeptide at preselected bases such that codons are generated that will translate into the desired amino acids.

Another means of increasing the number of carbohydrate moieties on the Hepatitis C infection polypeptide is by chemical or enzymatic coupling of glycosides to the polypeptide. See, e.g., WO 87/05330; and pp. 259-306 in Aplin and Wriston (1981) CRC Crit. Rev.

Biochem.

Removal of carbohydrate moieties present on the Hepatitis C infection polypeptide may be accomplished chemically or enzymatically or by mutational substitution of codons encoding for amino acid residues that serve as targets for glycosylation. Chemical deglycosylation techniques are applicable. See, e.g., Sojar and Bahl (1987) Arch. Biochem. Biophys. 259:52-57; and Edge, et al. (1981) Anal. Biochem. 118:131-137. Enzymatic cleavage of carbohydrate moieties on polypeptides can be achieved by the use of a variety of endo- and exo-glycosidases. See, e.g., Thotakura, et al. (1987) Meth. Enzymol. 138:350-359.

Another type of covalent modification of Hepatitis C infection comprises linking the Hepatitis C infection polypeptide to one of a variety of nonproteinaceous polymers, e.g., polyethylene glycol, polypropylene glycol, or polyoxyalkylenes, in the manner set forth in U.S. Pat. Nos. 4,640,835; 4,496,689; 4,301,144; 4,670,417; 4,791,192; or 4,179,337.

Hepatitis C infection polypeptides of the present invention may also be modified in a way to form chimeric molecules comprising a Hepatitis C infection polypeptide fused to another heterologous polypeptide or amino acid sequence. In one embodiment, such a chimeric molecule comprises a fusion of a Hepatitis C infection polypeptide with a tag polypeptide which provides an epitope to which an anti-tag antibody can selectively bind. The epitope tag is generally placed at the amino-or carboxyl-terminus of the Hepatitis C infection polypeptide. The presence of such epitope-tagged forms of a Hepatitis C infection polypeptide can be detected using an antibody against the tag polypeptide. Also, provision of the epitope tag enables the Hepatitis C infection polypeptide to be readily purified by affinity purification using an anti-tag antibody or another type of affinity matrix that binds to the epitope tag. In an alternative embodiment, the chimeric molecule may comprise a fusion of a Hepatitis C infection polypeptide with an immunoglobulin or a particular region of an immunoglobulin. For a bivalent form of the chimeric molecule, such a fusion could be to the Fe region of an IgG molecule.

Various tag polypeptides and their respective antibodies are available. Examples include poly-histidine (poly-his) or poly-histidine-glycine (poly-his-gly) tags; HIS6, and metal chelation tags, the flu HA tag polypeptide and its antibody 12CA5 (Field, et al. (1988) Mol. Cell. Biol. 8:2159-2165); the c-myc tag and the 8F9, 3C7, 6E10, G4, B7, and 9E10 antibodies thereto (Evan, et al. (1985) Mol. Cell. Biol. 5:3610-3616); and the Herpes Simplex virus glycoprotein D (gD) tag and its antibody (Paborsky, et al. (1990) Protein Engineering 3:547-553). Other tag polypeptides include the Flag-peptide (Hopp, et al. (1988) BioTechnology 6:1204-1210); the KT3 epitope peptide (Martin, et al. (1992) Science 255:192-194); tubulin epitope peptide (Skinner, et al. (1991) J. Biol. Chem. 266:15163-15166); and the T7 gene 10 protein peptide tag (Lutz-Freyermuth, et al. (1990) Proc. Nat'l Acad. Sci. USA 87:6393-6397).

Also included are other Hepatitis C infection proteins of the family, and Hepatitis C infection proteins from other organisms, which are cloned and expressed as outlined below. Thus, probe or degenerate polymerase chain reaction (PCR) primer sequences may be used to find other related Hepatitis C infection proteins from humans or other organisms. As will be appreciated by those in the art, particularly useful probe and/or PCR primer sequences include the unique areas of the Hepatitis C infection nucleic acid sequence. As is generally known in the art, preferred PCR primers are from about 15-35 nucleotides in length, with from about 20-30 being preferred, and may contain inosine as needed. The conditions for the PCR reaction are well known. See, e.g., Innis, PCR Protocols, supra.

In addition, as is outlined herein, Hepatitis C proteins can be made that are longer than those encoded by the nucleic acids of the Tables, e.g., by the elucidation of extended sequences, the addition of epitope or purification tags, the addition of other fusion sequences, etc.

Hepatitis C proteins may also be identified as being encoded by Hepatitis C nucleic acids. Thus, Heptatitis C proteins are encoded by nucleic acids that will hybridize to the sequences of the sequence listings, or their complements, as outlined herein.

Antibodies to Hepatitis C Infection Proteins

In a preferred embodiment, when the Hepatitis C infection protein is to be used to generate antibodies, e.g., for immunotherapy or immunodiagnosis, the Hepatitis C infection protein should share at least one epitope or determinant with the full length protein. By “epitope” or “determinant” herein is typically meant a portion of a protein which will generate and/or bind an antibody or T-cell receptor in the context of MHC. Thus, in most instances, antibodies made to a smaller Hepatitis C infection protein will be able to bind to the full-length protein, particularly linear epitopes. In a preferred embodiment, the epitope is unique; that is, antibodies generated to a unique epitope show little or no cross-reactivity.

Methods of preparing polyclonal antibodies are known to the skilled artisan (e.g., Coligan, supra; and Harlow and Lane, supra). Polyclonal antibodies can be raised in a mammal, e.g., by one or more injections of an immunizing agent and, if desired, an adjuvant. Typically, the immunizing agent and/or adjuvant will be injected in the mammal by multiple subcutaneous or intraperitoneal injections. The immunizing agent may include a protein encoded by a nucleic acid of the figures or fragment thereof or a fusion protein thereof. It may be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). The immunization protocol may be selected by one skilled in the art without undue experimentation.

The antibodies may, alternatively, be monoclonal antibodies. Monoclonal antibodies may be prepared using hybridoma methods, such as those described by Kohler and Milstein (1975) Nature 256:495-497. In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. The immunizing agent will typically include a polypeptide encoded by a nucleic acid of Tables 1A-15 or fragment thereof, or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (e.g., pp. 59-103 in Goding (1986) Monoclonal Antibodies: Principles and Practice Academic Press). Immortalized cell lines are usually transformed mammalian cells, particularly myeloma cells of rodent, bovine, primate, and human origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

In one embodiment, the antibodies are bispecific antibodies. Bispecific antibodies are monoclonal, preferably human or humanized, antibodies that have binding specificities for at least two different antigens or that have binding specificities for two epitopes on the same antigen. In one embodiment, one of the binding specificities is for a protein encoded by a nucleic acid Tables 1A-15 or a fragment thereof, the other one is for another antigen, and preferably for a cell-surface protein or receptor or receptor subunit, preferably one that is tumor specific. Alternatively, tetramer-type technology may create multivalent reagents.

In a preferred embodiment, the antibodies to Hepatitis C infection protein are capable of reducing or eliminating a biological function of a Hepatitis C infection protein, as is described below. That is, the addition of anti-Hepatitis C infection protein antibodies (either polyclonal or preferably monoclonal or oligoclonal) to Hepatitis C infected cells or tissues or cells and tissues secondarily affected by Hepatitis C infection, may reduce or eliminate the Hepatitis C infection and/or its secondary consequences. Generally, at least about 25% decrease in activity, growth, size, or the like is preferred, with at least about 50% being particularly preferred and about 95-100% decrease being especially preferred.

In a preferred embodiment the antibodies to the Hepatitis C infection proteins are humanized antibodies (e.g., Xenerex Biosciences, Medarex, Inc., Abgenix, Inc., Protein Design Labs, Inc.). Humanized forms of non-human (e.g., murine) antibodies are chimeric molecules of immunoglobulins, immunoglobulin chains, or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will typically comprise at least a portion of an immunoglobulin constant region (Fc), typically that of a human immunoglobulin (Jones, et al. (1986) Nature 321:522-525; Riechmann, et al. (1988) Nature 332:323-329; and Presta (1992) Curr. Op. Struct. Biol. 2:593-596). Humanization can be essentially performed following the method of Winter and co-workers (Jones, et al. (1986) Nature 321:522-525; Riechmann, et al. (1988) Nature 332:323-327; Verhoeyen, et al. (1988) Science 239:1534-1536), by substituting rodent CDRs or CDR sequences for corresponding sequences of a human antibody. Accordingly, such humanized antibodies are chimeric antibodies (U.S. Pat. No. 4,816,567), wherein substantially less than an intact human variable domain has been substituted by corresponding sequence from a non-human species.

Human-like antibodies can also be produced using phage display libraries (Hoogenboom and Winter (1992) J. Mol. Biol. 227:381-388; Marks, et al. (1991) J. Mol. Biol. 222:581-597) or human monoclonal antibodies (e.g., p. 77, Cole, et al. in Reisfeld and Sell (1985) Monoclonal Antibodies and Cancer Therapy Liss; and Boemer, et al. (1991) J. Immunol. 147:86-95). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in nearly all respects, including gene rearrangement, assembly, and antibody repertoire. This approach is described, e.g., in U.S. Pat. Nos. 5,545,807; 5,545,806; 5,569,825; 5,625,126; 5,633,425; 5,661,016, and in the following scientific publications: Marks, et al. (1992) Bio/Technology 10:779-783; Lonberg, et al. (1994) Nature 368:856-859; Morrison (1994) Nature 368:812-13; Fishwild, et al. (1996) Nature Biotechnology 14:845-851; Neuberger (1996) Nature Biotechnology 14:826; and Lonberg and Huszar (1995) Intern. Rev. Immunol. 13:65-93.

By immunotherapy is meant treatment of Hepatitis C infection and its secondary consequences with an antibody raised against Hepatitis C infection proteins. As used herein, immunotherapy can be passive or active. Passive immunotherapy as defined herein is the passive transfer of antibody to a recipient (patient). Active immunization is the induction of antibody and/or T-cell responses in a recipient (patient). Induction of an immune response is the result of providing the recipient with an antigen to which antibodies are raised. As appreciated by one of ordinary skill in the art, the antigen may be provided by injecting a polypeptide against which antibodies are desired to be raised into a recipient, or contacting the recipient with a nucleic acid capable of expressing the antigen and under conditions for expression of the antigen, leading to an immune response.

In a preferred embodiment the Hepatitis C infection proteins against which antibodies are raised are secreted proteins as described above. Without being bound by theory, antibodies used for treatment will typically bind and prevent the secreted protein from binding to its receptor, thereby inactivating the secreted Hepatitis C infection protein, e.g., in autocrine signaling.

In another preferred embodiment, the Hepatitis C infection protein to which antibodies are raised is a transmembrane protein. Without being bound by theory, antibodies used for treatment may bind the extracellular domain of the Hepatitis C infection protein and prevent it from binding to other proteins, such as circulating ligands or cell-associated molecules. The antibody may cause down-regulation of the transmembrane Hepatitis C infection protein. As will be appreciated by one of ordinary skill in the art, the antibody may be a competitive, non-competitive or uncompetitive inhibitor of protein binding to the extracellular domain of the Hepatitis C infection protein. The antibody is also an antagonist of the Hepatitis C infection protein. Further, the antibody prevents activation of the transmembrane Hepatitis C infection protein. The antibody may also be used to target or sensitize the cell to cytotoxic agents, including, but not limited to TNF-α, TNF-β, IL-1, INF-γ and IL-2, or chemotherapeutic agents including 5FU, vinblastine, actinomycin D, cisplatin, methotrexate, and the like. In some instances the antibody belongs to a sub-type that activates serum complement when complexed with the transmembrane protein thereby mediating cytotoxicity or antigen-dependent cytotoxicity (ADCC). Thus, Hepatitis C infection and its secondary consequences is treated by administering to a patient antibodies directed against the transmembrane Hepatitis C infection protein. Antibody-labeling may activate a co-toxin, localize a toxin payload, or otherwise provide means to locally ablate cells.

In another preferred embodiment, the antibody is conjugated to an effector moiety. The effector moiety can be a labeling moiety, e.g., a radioactive or fluorescent label, or a therapeutic moiety. In one aspect the therapeutic moiety is a small molecule that modulates the activity of the Hepatitis C infection protein. In another aspect the therapeutic moiety modulates the activity of molecules associated with or in close proximity to the Hepatitis C infection protein. The therapeutic moiety may inhibit enzymatic activity such as protease or collagenase or protein kinase activity associated with Hepatitis C infection and its secondary consequences. The effector may activate an endogenous physiological or immunological response.

In a preferred embodiment, the therapeutic moiety can also be a cytotoxic agent. In this method, targeting the cytotoxic agent to tissue or cells that are either directly infected with Hepatitis C or which are affected secondarily by the Hepatitis C infection, results in a reduction in the number of afflicted cells, thereby reducing symptoms associated with Hepatitis C infection and its secondary consequences. Cytotoxic agents are numerous and varied and include, but are not limited to, cytotoxic drugs or toxins or active fragments of such toxins. Suitable toxins and their corresponding fragments include diphtheria A chain, exotoxin A chain, ricin A chain, abrin A chain, curcin, crotin, saporin, maytansins, aurostatin, phenomycin, enomycin, and the like. Cytotoxic agents also include radiochemicals made by conjugating radioisotopes to antibodies raised against Hepatitis C infection proteins, or binding of a radionuclide to a chelating agent that has been covalently attached to the antibody. Targeting the therapeutic moiety to transmembrane Hepatitis C infection proteins not only serves to increase the local concentration of therapeutic moiety in the afflicted area, but also serves to reduce deleterious side effects that may be associated with the therapeutic moiety.

In another preferred embodiment, the Hepatitis C infection protein against which the antibodies are raised is an intracellular protein. In this case, the antibody may be conjugated to a protein which facilitates entry into the cell. In one case, the antibody enters the cell by endocytosis. In another embodiment, a nucleic acid encoding the antibody is administered to the individual or cell. Moreover, wherein the Hepatitis C infection protein can be targeted within a cell, e.g., the nucleus, an antibody thereto contains a signal for that target localization, e.g., a nuclear localization signal.

The antibodies to Hepatitis C afflicted cells and tissues of the invention specifically bind to Hepatitis C infection proteins. By “specifically bind” herein is meant that the antibodies bind to the protein with a K_{d} of at least about 0.1 mM, more usually at least about 1 μM, preferably at least about 0.1 μM or better, and most preferably, 0.01 μM or better. Selectivity of binding is also important.

Detection of Hepatitis C Infection Sequences for Diagnostic and Therapeutic Applications

In one aspect, the RNA expression levels of genes are determined for different cellular states in the Hepatitis C infection phenotype. Expression levels of genes in tissue from uninfected individuals and in Hepatitis C infected or affected tissue are evaluated to provide expression profiles. An expression profile of a particular cell state or point of development is essentially a “fingerprint” of the state. While two states may have any particular gene similarly expressed, the evaluation of a number of genes simultaneously allows the generation of a gene expression profile that is reflective of the state of the cell. By comparing expression profiles of cells in different states, information regarding which genes are important (including both up- and down-regulation of genes) in each of these states is obtained. Then, diagnosis may be performed or confirmed to determine whether a tissue sample has the gene expression profile of normal or cancerous tissue. This will provide for molecular diagnosis of related conditions.

“Differential expression,” or grammatical equivalents as used herein, refers to qualitative or quantitative differences in the temporal and/or cellular gene expression patterns within and among cells and tissue. Thus, a differentially expressed gene can qualitatively have its expression altered, including an activation or inactivation, in, e.g., normal versus Hepatitis C infected tissue. Genes may be turned on or turned off in a particular state, relative to another state thus permitting comparison of two or more states. A qualitatively regulated gene will exhibit an expression pattern within a state or cell type which is detectable by standard techniques. Some genes will be expressed in one state or cell type, but not in both. Alternatively, the difference in expression may be quantitative, e.g., in that expression is increased or decreased; e.g., gene expression is either upregulated, resulting in an increased amount of transcript, or downregulated, resulting in a decreased amount of transcript. The degree to which expression differs need only be large enough to quantify via standard characterization techniques as outlined below, such as by use of Affymetrix GeneChip™ expression arrays. See, Lockhart (1996) Nature Biotechnology 14:1675-1680. Other techniques include, but are not limited to, quantitative reverse transcriptase PCR, northern analysis, and RNase protection. As outlined above, preferably the change in expression (e.g., upregulation or downregulation) is at least about 50%, more preferably at least about 100%, more preferably at least about 150%, more preferably at least about 200%, with from 300 to at least 1000% being especially preferred.

Evaluation may be at the gene transcript, or the protein level. The amount of gene expression may be monitored using nucleic acid probes to the DNA or RNA equivalent of the gene transcript, and the quantification of gene expression levels, or, alternatively, the final gene product itself (protein) can be monitored, e.g., with antibodies to the Hepatitis C infection protein and standard immunoassays (ELISAs, etc.) or other techniques, including mass spectroscopy assays, 2D gel electrophoresis assays, etc. Proteins corresponding to Hepatitis C infection genes, e.g., those identified as being important in a Hepatitis C infection phenotype, can be evaluated in a diagnostic test for Hepatitis C infection and/or its secondary consequences, and certainly subsetting into responsive or non-responsive to specific treatment. In a preferred embodiment, gene expression monitoring is performed simultaneously on a number of genes. Multiple protein expression monitoring can be performed as well. Similarly, these assays may be performed on an individual basis as well.

In this embodiment, the Hepatitis C infection nucleic acid probes are attached to biochips as outlined herein for the detection and quantification of Hepatitis C infection sequences in a particular cell. The assays are further described below in the example. PCR techniques can be used to provide greater sensitivity.

In a preferred embodiment nucleic acids encoding the Hepatitis C infection protein are detected. Although DNA or RNA encoding the Hepatitis C infection protein may be detected, of particular interest are methods wherein an mRNA encoding a Hepatitis C infection protein is detected. Probes to detect mRNA can be a nucleotide/deoxynucleotide probe that is complementary to and hybridizes with the mRNA and includes, but is not limited to, oligonucleotides, cDNA or RNA. Probes also should contain a detectable label, as defined herein. In one method the mRNA is detected after immobilizing the nucleic acid to be examined on a solid support such as nylon membranes and hybridizing the probe with the sample. Following washing to remove non-specifically bound probe, the label is detected. In another method detection of the mRNA is performed in situ. In this method permeabilized cells or tissue samples are contacted with a detectably labeled nucleic acid probe for sufficient time to allow the probe to hybridize with target mRNA. Following washing to remove the non-specifically bound probe, the label is detected. For example a digoxygenin labeled riboprobe (RNA probe) that is complementary to the mRNA encoding a Hepatitis C infection protein is detected by binding the digoxygenin with an anti-digoxygenin secondary antibody and developed with nitro blue tetrazolium and 5-bromo-4-chloro-3-indoyl phosphate.

In a preferred embodiment, various proteins from the three classes of proteins as described herein (secreted, transmembrane or intracellular proteins) are used in diagnostic assays. The Hepatitis C infection proteins, antibodies, nucleic acids, modified proteins, and cells containing Hepatitis C infection sequences are used in diagnostic assays. This can be performed on an individual gene or corresponding polypeptide level. In a preferred embodiment, the expression profiles are used, preferably in conjunction with high throughput screening techniques to allow monitoring for expression profile genes and/or corresponding polypeptides.

As described and defined herein, Hepatitis C infection proteins, including intracellular, transmembrane, or secreted proteins, find use as markers of Hepatitis C infection or treatment response. Detection of these proteins in putative Hepatitis C infected tissue as well as in tissues that are affected secondarily by Hepatitis C infection, allows for detection or diagnosis of Hepatitis C infection and/or its secondary consequences. In one embodiment, antibodies are used to detect Hepatitis C infection proteins. A preferred method separates proteins from a sample by electrophoresis on a gel (typically a denaturing and reducing protein gel, but may be another type of gel, including isoelectric focusing gels and the like). Following separation of proteins, the Hepatitis C infection protein is detected, e.g., by immunoblotting with antibodies raised against the Hepatitis C infection protein. Methods of immunoblotting are well known to those of ordinary skill in the art.

In another preferred method, antibodies to the Hepatitis C infection protein find use in in situ imaging techniques, e.g., in histology (e.g., Asai, et al. (eds. 1993) Methods in Cell Biology: Antibodies in Cell Biology (vol. 37) Academic Press). In this method cells are contacted with from one to many antibodies to the Hepatitis C infection protein(s). Following washing to remove non-specific antibody binding, the presence of the antibody or antibodies is detected. In one embodiment the antibody is detected by incubating with a secondary antibody that contains a detectable label. In another method the primary antibody to the Hepatitis C infection protein(s) contains a detectable label, e.g., an enzyme marker that can act on a substrate. In another preferred embodiment each of multiple primary antibodies contains a distinct and detectable label. This method finds particular use in simultaneous screening for a plurality of Hepatitis C infection proteins. Many other histological and/or imaging techniques are also provided by the invention.

In a preferred embodiment the label is detected in a fluorometer which has the ability to detect and distinguish emissions of different wavelengths. In addition, a fluorescence activated cell sorter (FACS) can be used in the method.

In another preferred embodiment, antibodies find use in diagnosing Hepatitis C infection from blood, urine, sputum, serum, plasma, stool, and other samples. Such samples, therefore, are useful as samples to be probed or tested for the presence of Hepatitis C infection proteins. Antibodies can be used to detect a Hepatitis C infection protein by previously described immunoassay techniques including ELISA, immunoblotting (western blotting), immunoprecipitation, BIACORE technology and the like. Alternatively, the presence of antibodies may indicate an immune response against an endogenous infection.

In a preferred embodiment, in situ hybridization of labeled Hepatitis C infection nucleic acid probes to tissue arrays is done. For example, arrays of tissue samples, including Hepatitis C infection tissue and/or normal tissue, are made. In situ hybridization (see, e.g., Ausubel, supra) is then performed. When comparing the fingerprints between an individual and a standard, the skilled artisan can make a diagnosis, a prognosis, or a prediction based on the findings. It is further understood that the genes which indicate the diagnosis may differ from those which indicate the prognosis and molecular profiling of the condition of the cells may lead to distinctions between responsive or refractory conditions or may be predictive of outcomes.

In a preferred embodiment, the Hepatitis C infection proteins, antibodies, nucleic acids, modified proteins, and cells containing sequences associated with Hepatitis C infection and/or its secondary consequences are used in prognosis assays. As above, gene expression profiles can be generated that correlate to Hepatitis C infection and/or its secondary consequences, in terms of long term prognosis. Again, this may be done on either a protein or gene level, with the use of genes being preferred. As above, Hepatitis C infection probes may be attached to biochips for the detection and quantification of Hepatitis C infection sequences in a tissue or patient. The assays proceed as outlined above for diagnosis. PCR methods may provide more sensitive and accurate quantification.

Assays for Therapeutic Compounds

In a preferred embodiment members of the proteins, nucleic acids, and antibodies as described herein are used in drug screening assays. The Hepatitis C infection proteins, antibodies, nucleic acids, modified proteins, and cells containing Hepatitis C infection sequences are used in drug screening assays or by evaluating the effect of drug candidates on a “gene expression profile” or expression profile of polypeptides. In a preferred embodiment, the expression profiles are used, preferably in conjunction with high throughput screening techniques to allow monitoring for expression profile genes after treatment with a candidate agent. See, e.g., Zlokarnik, et al. (1998) Science 279:84-88; and Heid (1996) Genome Res. 6:986-994.

In a preferred embodiment, the Hepatitis C infection proteins, antibodies, nucleic acids, modified proteins, and cells containing the native or modified Hepatitis C infection proteins are used in screening assays. That is, the present invention provides novel methods for screening for compositions which modulate the Hepatitis C infection phenotype or an identified physiological function of a Hepatitis C infection protein. As above, this can be done on an individual gene level or by evaluating the effect of drug candidates on a “gene expression profile”. In a preferred embodiment, the expression profiles are used, preferably in conjunction with high throughput screening techniques to allow monitoring for expression profile genes after treatment with a candidate agent, see Zlokarnik, supra.

Having identified the differentially expressed genes herein, a variety of assays may be executed. In a preferred embodiment, assays may be run on an individual gene or protein level. That is, having identified a particular gene as up regulated during Hepatitis C infection, test compounds can be screened for the ability to modulate gene expression or for binding to the Hepatitis C infection protein. “Modulation” thus includes both an increase and a decrease in gene expression. The preferred amount of modulation will depend on the original change of the gene expression in normal versus tissue experiencing Hepatitis C infection and its secondary consequences, with changes of at least about 10%, preferably about 50%, more preferably about 100-300%, and in some embodiments about 300-1000% or greater. Thus, if a gene exhibits a 4-fold increase in tissue experiencing hepatitis C infection or its secondary consequences compared to normal tissue, a decrease of about four-fold is often desired; similarly, a 10-fold decrease in tissue experiencing Hepatitis C infection or its secondary consequences compared to normal tissue often provides a target value of a 10-fold increase in expression to be induced by the test compound.

The amount of gene expression may be monitored using nucleic acid probes and the quantification of gene expression levels, or, alternatively, the gene product itself can be monitored, e.g., through the use of antibodies to the Hepatitis C infection protein and standard immunoassays. Proteomics and separation techniques may also allow quantification of expression.

In a preferred embodiment, gene expression or protein monitoring of a number of entities, e.g., an expression profile, is monitored simultaneously. Such profiles will typically involve a plurality of those entities described herein.

In this embodiment, the Hepatitis C infection nucleic acid probes are attached to biochips as outlined herein for the detection and quantification of Hepatitis C infection sequences in a particular cell. Alternatively, PCR may be used. Thus, a series, e.g., of microtiter plate, may be used with dispensed primers in desired wells. A PCR reaction can then be performed and analyzed for each well.

Modulators of Testicular Cancer

Expression monitoring can be performed to identify compounds that modify the expression of one or more Hepatitis C infection-associated sequences, e.g., a polynucleotide sequence set out in Tables 1A-15. Generally, in a preferred embodiment, a test modulator is added to the cells prior to analysis. Moreover, screens are also provided to identify agents that modulate Hepatitis C infection and its secondary consequences, modulate Hepatitis C infection proteins, bind to a Hepatitis C infection protein, or interfere with the binding of a Hepatitis C infection protein and an antibody or other binding partner.

The term “test compound” or “drug candidate” or “modulator” or grammatical equivalents as used herein describes any molecule, e.g., protein, oligopeptide, small organic molecule, polysaccharide, polynucleotide, etc., to be tested for the capacity to directly or indirectly alter the Hepatitis C infection phenotype (direct or indirect) or the expression of a Hepatitis C infection sequence, e.g., a nucleic acid or protein sequence. In preferred embodiments, modulators alter expression profiles, or expression profile nucleic acids or proteins provided herein. In one embodiment, the modulator suppresses a Hepatitis C infection phenotype, e.g., to a normal tissue fingerprint. In another embodiment, a modulator induced a Hepatitis C infection phenotype. Generally, a plurality of assay mixtures are run in parallel with different agent concentrations to obtain a differential response to the various concentrations. Typically, one of these concentrations serves as a negative control, e.g., at zero concentration or below the level of detection.

Drug candidates encompass numerous chemical classes, though typically they are organic molecules, preferably small organic compounds having a molecular weight of more than 100 and less than about 2,500 daltons. Preferred small molecules are less than 2000, or less than 1500 or less than 1000 or less than 500 D. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs, or combinations thereof. Particularly preferred are peptides or orally active compounds.

In one aspect, a modulator will neutralize the effect of a Hepatitis C infection protein. By “neutralize” is meant that activity of a protein is inhibited or blocked with a consequent effect on the cell.

In certain embodiments, combinatorial libraries of potential modulators will be screened for an ability to bind to a Hepatitis C infection polypeptide or to modulate activity. Conventionally, new chemical entities with useful properties are generated by identifying a chemical compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

In one preferred embodiment, high throughput screening methods involve providing a library containing a large number of potential therapeutic compounds (candidate compounds). Such “combinatorial chemical libraries” are then screened in one or more assays to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis by combining a number of chemical “building blocks” such as reagents. For example, a linear combinatorial chemical library, such as a polypeptide (e.g., mutein) library, is formed by combining a set of chemical building blocks called amino acids in every possible way for a given compound length (e.g., the number of amino acids in a polypeptide compound). Millions of chemical compounds can be synthesized through such combinatorial mixing of chemical building blocks. See Gallop, et al. (1994) J. Med. Chem. 37:1233-1251.

Preparation and screening of combinatorial chemical libraries is well known. Such combinatorial chemical libraries include, but are not limited to, peptide libraries (see, e.g., U.S. Pat. No. 5,010,175, Furka (1991) Pept. Prot. Res. 37:487-493, Houghton, et al. (1991) Nature 354:84-88), peptoids (PCT Publication No WO 91/19735), encoded peptides (PCT Publication WO 93/20242), random bio-oligomers (PCT Publication WO 92/00091), benzodiazepines (U.S. Pat. No. 5,288,514), diversomers such as hydantoins, benzodiazepines and dipeptides (Hobbs, et al. (1993) Proc. Nat'l Acad. Sci. USA 90:6909-6913, vinylogous polypeptides (Hagihara, et al. (1992) J. Amer. Chem. Soc. 114:6568-xxx), nonpeptidal peptidomimetics with a Beta-D-Glucose scaffolding (Hirschmann, et al. (1992) J. Amer. Chem. Soc. 114:9217-9218), analogous organic syntheses of small compound libraries (Chen, et al. (1994) J. Amer. Chem. Soc. 116:2661-xxx), oligocarbamates (Cho, et al. (1993) Science 261:1303-1305), and/or peptidyl phosphonates (Campbell, et al. (1994) J. Org. Chem. 59:658-xxx). See, generally, Gordon, et al. (1994) J. Med. Chem. 37:1385-1401, nucleic acid libraries (see, e.g., Stratagene, Corp.), peptide nucleic acid libraries (see, e.g., U.S. Pat. No. 5,539,083), antibody libraries (see, e.g., Vaughn, et al. (1996) Nature Biotechnology 14(3):309-314, and PCT/US96/10287), carbohydrate libraries (see, e.g., Liang, et al. (1996) Science 274:1520-1522, and U.S. Pat. No. 5,593,853), and small organic molecule libraries (see, e.g., benzodiazepines, page 33 Baum (Jan. 18, 1993) C&ENews; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514; and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.), which mimic the manual synthetic operations performed by a chemist. The above devices are suitable for use with the present invention. The nature and implementation of modifications to these devices (if any) so that they can operate as discussed herein will be apparent to persons skilled in the relevant art. In addition, numerous combinatorial libraries are themselves commercially available. See, e.g., ComGenex, Princeton, N.J.; Asinex, Moscow, Ru; Tripos, Inc., St. Louis, Mo.; ChemStar, Ltd, Moscow, RU; 3D Pharmaceuticals, Exton, Pa.; Martek Biosciences, Columbia, Md.; etc.

Assays to identify modulators are amenable to high throughput screening. Preferred assays thus detect enhancement or inhibition of Hepatitis C infection gene transcription, inhibition or enhancement of polypeptide expression, and inhibition or enhancement of polypeptide activity.

High throughput assays for the presence, absence, quantification, or other properties of particular nucleic acids or protein products are well known to those of skill in the art. Similarly, binding assays and reporter gene assays are similarly well known. Thus, e.g., U.S. Pat. No. 5,559,410 discloses high throughput screening methods for proteins, U.S. Pat. No. 5,585,639 discloses high throughput screening methods for nucleic acid binding (e.g., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc., Fullerton, Calif.; Precision Systems, Inc., Natick, Mass., etc.). These systems typically automate entire procedures, including sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols for various high throughput systems. Thus, e.g., Zymark Corp. provides technical bulletins describing screening systems for detecting the modulation of gene transcription, ligand binding, and the like.

In one embodiment, modulators are proteins, often naturally occurring proteins or fragments of naturally occurring proteins. Thus, e.g., cellular extracts containing proteins, or random or directed digests of proteinaceous cellular extracts, may be used. In this way libraries of proteins may be made for screening in the methods of the invention. Particularly preferred in this embodiment are libraries of bacterial, fungal, viral, and mammalian proteins, with the latter being preferred, and human proteins being especially preferred. Particularly useful test compound will be directed to the class of proteins to which the target belongs, e.g., substrates for enzymes or ligands and receptors.

In a preferred embodiment, modulators are peptides of from about 5-30 amino acids, with from about 5-20 amino acids being preferred, and from about 7-15 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate nucleotide or amino acid variations. The synthetic process can be designed to generate randomized proteins or nucleic acids, to allow the formation of the possible combinations over the length of the sequence, thus forming a library of randomized candidate bioactive proteinaceous agents.

In one embodiment, the library is randomized, with few or no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, e.g., of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines, or histidines for phosphorylation sites, etc., or to purines, etc.

Modulators of Hepatitis C infection and its secondary consequences can also be nucleic acids, as defined above.

As described above generally for proteins, nucleic acid modulating agents may be naturally occurring nucleic acids, random nucleic acids, or “biased” random nucleic acids. For example, digests of prokaryotic or eukaryotic genomes may be used as is outlined above for proteins.

In a preferred embodiment, the candidate compounds are organic chemical moieties, a wide variety of which are available in the literature.

After a candidate agent has been added and the cells allowed to incubate for some period of time, the sample containing a target sequence to be analyzed is added to the biochip. If required, the target sequence is prepared using known techniques. For example, the sample may be treated to lyse the cells, using known lysis buffers, electroporation, etc., with purification and/or amplification such as PCR performed as appropriate. For example, an in vitro transcription with labels covalently attached to the nucleotides is performed. Generally, the nucleic acids are labeled with biotin-FITC or PE, or with cy3 or cy5.

In a preferred embodiment, the target sequence is labeled with, e.g., a fluorescent, a chemiluminescent, a chemical, or a radioactive signal, to provide a means of detecting the target sequence's specific binding to a probe. The label also can be an enzyme, such as, alkaline phosphatase or horseradish peroxidase, which when provided with an appropriate substrate produces a product that can be detected. Alternatively, the label can be a labeled compound or small molecule, such as an enzyme inhibitor, that binds but is not catalyzed or altered by the enzyme. The label also can be a moiety or compound, such as, an epitope tag or biotin which specifically binds to streptavidin. For the example of biotin, the streptavidin is labeled as described above, thereby, providing a detectable signal for the bound target sequence. Unbound labeled streptavidin is typically removed prior to analysis.

These assays can be direct hybridization assays or can comprise “sandwich assays”, which include the use of multiple probes, as is generally outlined in U.S. Pat. Nos. 5,681,702, 5,597,909, 5,545,730, 5,594,117, 5,591,584, 5,571,670, 5,580,731, 5,571,670, 5,591,584, 5,624,802, 5,635,352, 5,594,118, 5,359,100, 5,124,246 and 5,681,697, all of which are hereby incorporated by reference. In this embodiment, in general, the target nucleic acid is prepared as outlined above, and then added to the biochip comprising a plurality of nucleic acid probes, under conditions that allow the formation of a hybridization complex.

A variety of hybridization conditions may be used in the present invention, including high, moderate, and low stringency conditions as outlined above. The assays are generally run under stringency conditions which allows formation of the label probe hybridization complex only in the presence of target. Stringency can be controlled by altering a step parameter that is a thermodynamic variable, including, but not limited to, temperature, formamide concentration, salt concentration, chaotropic salt concentration pH, organic solvent concentration, etc.

These parameters may also be used to control non-specific binding, as is generally outlined in U.S. Pat. No. 5,681,697. Thus it may be desirable to perform certain steps at higher stringency conditions to reduce non-specific binding.

The reactions outlined herein may be accomplished in many ways. Components of the reaction may be added simultaneously, or sequentially, in different orders, with preferred embodiments outlined below. In addition, the reaction may include a variety of other reagents. These include salts, buffers, neutral proteins, e.g., albumin, detergents, etc., which may be used to facilitate optimal hybridization and detection, and/or reduce non-specific or background interactions. Reagents that otherwise improve the efficiency of the assay, such as protease inhibitors, nuclease inhibitors, anti-microbial agents, etc., may also be used as appropriate, depending on the sample preparation methods and purity of the target.

The assay data are analyzed to determine the expression levels, and changes in expression levels as between states, of individual genes, forming a gene expression profile.

Screens are performed to identify modulators of the Hepatitis C infection phenotype. In one embodiment, screening is performed to identify modulators that can induce or suppress a particular expression profile, thus preferably generating the associated phenotype. In another embodiment, e.g., for diagnostic applications, having identified differentially expressed genes important in a particular state, screens can be performed to identify modulators that alter expression of individual genes. In an another embodiment, screening is performed to identify modulators that alter a biological function of the expression product of a differentially expressed gene. Again, having identified the importance of a gene in a particular state, screens are performed to identify agents that bind and/or modulate the biological activity of the gene product.

In addition screens can be done for genes that are induced in response to a candidate agent. After identifying a modulator based upon its ability to suppress an expression pattern associated with Hepatitis C infection, leading to a normal expression pattern, or to modulate a single Hepatitis C infection gene expression profile so as to mimic the expression of the gene from normal tissue, a screen as described above can be performed to identify genes that are specifically modulated in response to the agent. Comparing expression profiles between normal tissue and agent treated tissue experiencing Hepatitis C infection or its secondary consequences reveals genes that are not expressed in normal tissue or in tissue experiencing Hepatitis C infection or its secondary consequences, but are expressed in agent treated tissue. These agent-specific sequences can be identified and used by methods described herein for Hepatitis C infection genes or proteins. In particular these sequences and the proteins they encode find use in marking or identifying agent treated cells. In addition, antibodies can be raised against the agent induced proteins and used to target novel therapeutics to the treated sample of tissue experiencing Hepatitis C infection or its secondary consequences.

Thus, in one embodiment, a test compound is administered to a population of cells, that have an associated Hepatitis C infection expression profile. By “administration” or “contacting” herein is meant that the candidate agent is added to the cells in such a manner as to allow the agent to act upon the cell, whether by uptake and intracellular action, or by action at the cell surface. In some embodiments, nucleic acid encoding a proteinaceous candidate agent (e.g., a peptide) may be put into a viral construct such as an adenoviral or retroviral construct, and added to the cell, such that expression of the peptide agent is accomplished, e.g., PCT US97/01019. Regulatable gene therapy systems can also be used.

Once the test compound has been administered to the cells, the cells can be washed if desired and are allowed to incubate under preferably physiological conditions for some period of time. The cells are then harvested and a new gene expression profile is generated, as outlined herein.

Thus, e.g., the tissue experiencing Hepatitis C infection or it secondary consequences may be screened for agents that modulate, e.g., induce or suppress the Hepatitis C infection phenotype. A change in at least one gene, preferably many, of the expression profile indicates that the agent has an effect on Hepatitis C infection. By defining such a signature for the Hepatitis C infection phenotype, screens for new drugs that alter the phenotype can be devised. With this approach, the drug target need not be known and need not be represented in the original expression screening platform, nor does the level of transcript for the target protein need to change.

In a preferred embodiment, as outlined above, screens may be done on individual genes and gene products (proteins). That is, having identified a particular differentially expressed gene as important in a particular state, screening of modulators of either the expression of the gene or the gene product itself can be done. The gene products of differentially expressed genes are sometimes referred to herein as “Hepatitis C infection proteins” or a “Hepatitis C infection modulatory protein”. The Hepatitis C infection modulatory protein may be a fragment, or alternatively, be the full length protein to the fragment encoded by the nucleic acids of the Tables. Preferably, the Hepatitis C infection modulatory protein is a fragment. In a preferred embodiment, the Hepatitis C infection amino acid sequence which is used to determine sequence identity or similarity is encoded by a nucleic acid of Tables 1A-15. In another embodiment, the sequences are naturally occurring allelic variants of a protein encoded by a nucleic acid of Tables 1A-15. In another embodiment, the sequences are sequence variants as further described herein.

Preferably, the Hepatitis C infection modulatory protein is a fragment of about 14-24 amino acids long. More preferably the fragment is a soluble fragment. Preferably, the fragment includes a non-transmembrane region. In a preferred embodiment, the fragment has an N-terminal Cys to aid in solubility. In one embodiment, the C-terminus of the fragment is kept as a free acid and the N-terminus is a free amine to aid in coupling, e.g., to cysteine.

In one embodiment the Hepatitis C infection proteins are conjugated to an immunogenic agent as discussed herein. In one embodiment the Hepatitis C infection protein is conjugated to BSA.

Measurements of Hepatitis C infection polypeptide activity, or the Hepatitis C infection phenotype can be performed using a variety of assays. For example, the effects of the test compounds upon the function of the Hepatitis C infection polypeptides can be measured by examining parameters described above. A suitable physiological change that affects activity can be used to assess the influence of a test compound on the polypeptides of this invention. When the functional consequences are determined using intact cells or animals, one can also measure a variety of effects such as, changes in intracellular second messengers such as cGMP. In the assays of the invention, mammalian Hepatitis C infection polypeptide is typically used, e.g., mouse, preferably human.

Assays to identify compounds with modulating activity can be performed in vitro. For example, a Hepatitis C infection polypeptide is first contacted with a potential modulator and incubated for a suitable amount of time, e.g., from 0.5 to 48 hours. In one embodiment, the Hepatitis C infection polypeptide levels are determined in vitro by measuring the level of protein or mRNA. The level of protein is measured, e.g., using immunoassays such as western blotting, ELISA, and the like with an antibody that selectively binds to the Hepatitis C infection polypeptide or a fragment thereof. For measurement of mRNA, amplification, e.g., using PCR, LCR, or hybridization assays, e.g., northern hybridization, RNAse protection, or dot blotting, are preferred. The level of protein or mRNA is detected using directly or indirectly labeled detection agents, e.g., fluorescently or radioactively labeled nucleic acids, radioactively or enzymatically labeled antibodies, and the like, as described herein.

Alternatively, a reporter gene system can be devised using the Hepatitis C infection protein promoter operably linked to a reporter gene such as luciferase, green fluorescent protein, CAT, or β-gal. The reporter construct is typically transfected into a cell. After treatment with a potential modulator, the amount of reporter gene transcription, translation, or activity is measured according to standard techniques.

In a preferred embodiment, as outlined above, screens may be done on individual genes and gene products (proteins). That is, having identified a particular differentially expressed gene as important in a particular state, screening of modulators of the expression of the gene or the gene product itself can be done. The gene products of differentially expressed genes are sometimes referred to herein as “Hepatitis C infection proteins.” The Hepatitis C infection protein may be a fragment, or alternatively, be the full length protein to a fragment shown herein.

In one embodiment, screening for modulators of expression of specific genes is performed. Typically, the expression of only one or a few genes are evaluated. In another embodiment, screens are designed to first find compounds that bind to differentially expressed proteins. These compounds are then evaluated for the ability to modulate differentially expressed activity. Moreover, once initial candidate compounds are identified, variants can be further screened to better evaluate structure activity relationships.

In a preferred embodiment, binding assays are done. In general, purified or isolated gene product is used; that is, the gene products of one or more differentially expressed nucleic acids are made. For example, antibodies are generated to the protein gene products, and standard immunoassays are run to determine the amount of protein present. Alternatively, cells comprising the Hepatitis C infection proteins can be used in the assays.

Thus, in a preferred embodiment, the methods comprise combining a Hepatitis C infection protein and a candidate compound, and determining the binding of the compound to the Hepatitis C infection protein. Preferred embodiments utilize the human Hepatitis C infection protein, although other mammalian proteins may also be used, e.g., for the development of animal models of human disease. In some embodiments, as outlined herein, variant or derivative Hepatitis C infection proteins may be used.

Generally, in a preferred embodiment of the methods herein, the Hepatitis C infection protein or the candidate agent is non-diffusably bound to an insoluble support having isolated sample receiving areas (e.g., a microtiter plate, an array, etc.). The insoluble supports may be made of any composition to which the compositions can be bound, is readily separated from soluble material, and is otherwise compatible with the overall method of screening. The surface of such supports may be solid or porous and of any convenient shape. Examples of suitable insoluble supports include microtiter plates, arrays, membranes, and beads. These are typically made of glass, plastic (e.g., polystyrene), polysaccharides, nylon, or nitrocellulose, teflon™, etc. Microtiter plates and arrays are especially convenient because a large number of assays can be carried out simultaneously, using small amounts of reagents and samples. The particular manner of binding of the composition is not crucial so long as it is compatible with the reagents and overall methods of the invention, maintains the activity of the composition and is nondiffusable. Preferred methods of binding include the use of antibodies (which do not sterically block either the ligand binding site or activation sequence when the protein is bound to the support), direct binding to “sticky” or ionic supports, chemical crosslinking, the synthesis of the protein or agent on the surface, etc. Following binding of the protein or agent, excess unbound material is removed by washing. The sample receiving areas may then be blocked through incubation with bovine serum albumin (BSA), casein, or other innocuous protein or other moiety.

In a preferred embodiment, the Hepatitis C infection protein is bound to the support, and a test compound is added to the assay. Alternatively, the candidate agent is bound to the support and the Hepatitis C infection protein is added. Novel binding agents include specific antibodies, non-natural binding agents identified in screens of chemical libraries, peptide analogs, etc. Of particular interest are screening assays for agents that have a low toxicity for human cells. A wide variety of assays may be used for this purpose, including labeled in vitro protein-protein binding assays, electrophoretic mobility shift assays, immunoassays for protein binding, functional assays (phosphorylation assays, etc.), and the like.

The determination of the binding of the test modulating compound to the Hepatitis C infection protein may be done in a number of ways. In a preferred embodiment, the compound is labeled, and binding determined directly, e.g., by attaching all or a portion of the Hepatitis C infection protein to a solid support, adding a labeled candidate agent (e.g., a fluorescent label), washing off excess reagent, and determining whether the label is present on the solid support. Various blocking and washing steps may be utilized as appropriate.

In some embodiments, just one of the components is labeled, e.g., the proteins (or proteinaceous candidate compounds) can be labeled. Alternatively, more than one component can be labeled with different labels, e.g., ¹²⁵I for the proteins and a fluorophor for the compound. Proximity reagents, e.g., quenching or energy transfer reagents are also useful.

In one embodiment, the binding of the test compound is determined by competitive binding assay. The competitor is a binding moiety known to bind to the target molecule (e.g., a Hepatitis C infection protein), such as an antibody, peptide, binding partner, ligand, etc. Under certain circumstances, there may be competitive binding between the compound and the binding moiety, with the binding moiety displacing the compound. In one embodiment, the test compound is labeled. Either the compound, or the competitor, or both, is added first to the protein for a time sufficient to allow binding, if present. Incubations may be performed at a temperature which facilitates optimal activity, typically between 4-40° C. Incubation periods are typically optimized, e.g., to facilitate rapid high throughput screening. Typically between 0.1 and 1 hour will be sufficient. Excess reagent is generally removed or washed away. The second component is then added, and the presence or absence of the labeled component is followed, to indicate binding.

In a preferred embodiment, the competitor is added first, followed by the test compound. Displacement of the competitor is an indication that the test compound is binding to the Hepatitis C infection protein and thus is capable of binding to, and potentially modulating, the activity of the Hepatitis C infection protein. In this embodiment, either component can be labeled. Thus, e.g., if the competitor is labeled, the presence of label in the wash solution indicates displacement by the agent. Alternatively, if the test compound is labeled, the presence of the label on the support indicates displacement.

In an alternative embodiment, the test compound is added first, with incubation and washing, followed by the competitor. The absence of binding by the competitor may indicate that the test compound is bound to the Hepatitis C infection protein with a higher affinity. Thus, if the test compound is labeled, the presence of the label on the support, coupled with a lack of competitor binding, may indicate that the test compound is capable of binding to the Hepatitis C infection protein.

In a preferred embodiment, the methods comprise differential screening to identify agents that are capable of modulating the activity of the Hepatitis C infection proteins. In this embodiment, the methods comprise combining a Hepatitis C infection protein and a competitor in a first sample. A second sample comprises a test compound, a Hepatitis C infection protein, and a competitor. The binding of the competitor is determined for both samples, and a change, or difference in binding between the two samples indicates the presence of an agent capable of binding to the Hepatitis C infection protein and potentially modulating its activity. That is, if the binding of the competitor is different in the second sample relative to the first sample, the agent is capable of binding to the Hepatitis C infection protein.

Alternatively, differential screening is used to identify drug candidates that bind to the native Hepatitis C infection protein, but cannot bind to modified Hepatitis C infection proteins. The structure of the Hepatitis C infection protein may be modeled, and used in rational drug design to synthesize agents that interact with that site. Drug candidates that affect the activity of a Hepatitis C infection protein are also identified by screening drugs for the ability to either enhance or reduce the activity of the protein.

Positive controls and negative controls may be used in the assays. Preferably control and test samples are performed in at least triplicate to obtain statistically significant results. Incubation of all samples is for a time sufficient for the binding of the agent to the protein. Following incubation, samples are washed free of non-specifically bound material and the amount of bound, generally labeled agent determined. For example, where a radiolabel is employed, the samples may be counted in a scintillation counter to determine the amount of bound compound.

A variety of other reagents may be included in the screening assays. These include reagents like salts, neutral proteins, e.g., albumin, detergents, etc., which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in an order that provides for the requisite binding.

In a preferred embodiment, the invention provides methods for screening for a compound capable of modulating the activity of a Hepatitis C infection protein. The methods comprise adding a test compound, as defined above, to a cell comprising Hepatitis C infection proteins. Many cell types may be used. The cells may contain a recombinant nucleic acid that encodes a Hepatitis C infection protein. In a preferred embodiment, a library of candidate agents are tested on a plurality of cells. In one aspect, the assays are evaluated in the presence or absence or previous or subsequent exposure of physiological signals, e.g., hormones, antibodies, peptides, antigens, cytokines, growth factors, action potentials, pharmacological agents including chemotherapeutics, radiation, carcinogenics, or other cells (e.g., cell-cell contacts). In another example, the determinations are determined at different stages of the cell cycle process.

In this way, compounds that modulate Hepatitis C infection agents are identified.

Compounds with pharmacological activity are able to enhance or interfere with the activity of the Hepatitis C infection protein. Once identified, similar structures are evaluated to identify critical structural feature of the compound.

In one embodiment, a method of inhibiting the consequences of Hepatitis C infection is provided. The method comprises administration of an inhibitor of processes that occur as a secondary consequence of Hepatitis C infection. In a further embodiment, methods of treating cells or tissues infected with Hepatitis C are provided. The method comprises administration of a inhibitor of Hepatitis C infection.

In one embodiment, a Hepatitis C infection inhibitor is an antibody as discussed above. In another embodiment, the Hepatitis C infection inhibitor is an antisense molecule.

Polynucleotide Modulators of Hepatitis C Infection and/or its Secondary Consequences Antisense Polynucleotides

In certain embodiments, the activity of a Hepatitis C infection-associated protein is down-regulated, or entirely inhibited, by the use of antisense polynucleotide, e.g., a nucleic acid complementary to, and which can preferably hybridize specifically to, a coding mRNA nucleic acid sequence, e.g., a Hepatitis C infection protein mRNA, or a subsequence thereof. Binding of the antisense polynucleotide to the mRNA reduces the translation and/or stability of the mRNA.

In the context of this invention, antisense polynucleotides can comprise naturally-occurring nucleotides, or synthetic species formed from naturally-occurring subunits or their close homologs. Antisense polynucleotides may also have altered sugar moieties or inter-sugar linkages. Exemplary among these are the phosphorothioate and other sulfur containing species which are known for use in the art. Analogs are comprehended by this invention so long as they function effectively to hybridize with the Hepatitis C infection protein mRNA. See, e.g., Isis Pharmaceuticals, Carlsbad, Calif.; Sequitor, Inc., Natick, Mass.

Such antisense polynucleotides can readily be synthesized using recombinant means, or can be synthesized in vitro. Equipment for such synthesis is sold by several vendors, including Applied Biosystems. The preparation of other oligonucleotides such as phosphorothioates and alkylated derivatives is also well known.

Antisense molecules as used herein include antisense or sense oligonucleotides. Sense oligonucleotides can, e.g., be employed to block transcription by binding to the anti-sense strand. The antisense and sense oligonucleotide comprise a single-stranded nucleic acid sequence (either RNA or DNA) capable of binding to target mRNA (sense) or DNA (antisense) sequences for Hepatitis C infection molecules. A preferred antisense molecule is for a Hepatitis C infection sequences in Tables 1A-15, or for a ligand or activator thereof. Antisense or sense oligonucleotides, according to the present invention, comprise a fragment generally at least about 14 nucleotides, preferably from about 14 to 30 nucleotides. The ability to derive an antisense or a sense oligonucleotide, based upon a cDNA sequence encoding a given protein is described in, e.g., Stein and Cohen (1988) Cancer Res. 48:2659—2668; and van der Krol, et al. (1988) BioTechniques 6:958-976).

RNA interference is a mechanism to suppress gene expression in a sequence specific manner. See, e.g., Brumelkamp, et al. (2002) Sciencexpress (Mar. 21, 2002); Sharp (1999) Genes Dev. 13:139-141; and Cathew (2001) Curr. Op. Cell Biol. 13:244-248. In mammalian cells, short, e.g., 21 nt, double stranded small interfering RNAs (siRNA) have been shown to be effective at inducing an RNAi response. See, e.g., Elbashir, et al. (2001) Nature 411:494-498. The mechanism may be used to downregulate expression levels of identified genes, e.g., treatment of or validation of relevance to disease.

Ribozymes

In addition to antisense polynucleotides, ribozymes can be used to target and inhibit transcription of Hepatitis C infection-associated nucleotide sequences. A ribozyme is an RNA molecule that catalytically cleaves other RNA molecules. Different kinds of ribozymes have been described, including group I ribozymes, hammerhead ribozymes, hairpin ribozymes, RNase P, and axhead ribozymes. See, e.g., Castanotto, et al. (1994) Adv. in Pharmacology 25:289-317.

General features of hairpin ribozymes are described, e.g., in Hampel, et al. (1990) Nuc. Acids Res. 18:299-304; European Patent Publication No. 0 360 257; U.S. Pat. No. 5,254,678. Methods of preparation are available. See, e.g., WO 94/26877; Yu, et al. (1993) Proc. Nat'l Acad. Sci. USA 90:6340-6344; Yamada, et al. (1994) Human Gene Therapy 1:39-45; Leavitt, et al. (1995) Proc. Nat'l Acad. Sci. USA 92:699-703; Leavitt, et al. (1994) Human Gene Therapy 5:1151-120; and Yamada, et al. (1994) Virology 205: 121-126.

Polynucleotide modulators of Hepatitis C infection may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell. Alternatively, a polynucleotide modulator of Hepatitis C infection may be introduced into a cell containing the target nucleic acid sequence, e.g., by formation of an polynucleotide-lipid complex, as described in WO 90/10448. It is understood that the use of antisense molecules or knock out and knock in models may also be used in screening assays as discussed above, in addition to methods of treatment.

Thus, in one embodiment, methods of modulating Hepatitis C infection in cells or organisms are provided. In one embodiment, the methods comprise administering to a cell an anti-Hepatitis C infection antibody that reduces or eliminates the biological activity of an endogenous Hepatitis C infection protein. Alternatively, the methods comprise administering to a cell or organism a recombinant nucleic acid encoding a Hepatitis C infection protein. This may be accomplished in any number of ways. In a preferred embodiment, e.g., when the Hepatitis C infection sequence is down-regulated during the course of Hepatitis C infection, such state may be reversed by increasing the amount of Hepatitis C infection associated gene product in the cell. This can be accomplished, e.g., by overexpressing the endogenous Hepatitis C infection gene or administering a gene encoding the Hepatitis C infection sequence, using known gene-therapy techniques. In a preferred embodiment, the gene therapy techniques include the incorporation of the exogenous gene using enhanced homologous recombination (EHR), e.g., as described in PCT/US93/03868, hereby incorporated by reference in its entirety. Alternatively, e.g., when the Hepatitis C infection sequence is up-regulated during Hepatitis C infection, the activity of the endogenous Hepatitis C infection gene is decreased, e.g., by the administration of a Hepatitis C infection antisense nucleic acid.

In one embodiment, the Hepatitis C infection proteins of the present invention may be used to generate polyclonal and monoclonal antibodies to proteins associated with Hepatitis C infection. Similarly, the Hepatitis C infection proteins can be coupled, using standard technology, to affinity chromatography columns. These columns may then be used to purify Hepatitis C infection antibodies useful for production, diagnostic, or therapeutic purposes. In a preferred embodiment, the antibodies are generated to epitopes unique to a Hepatitis C infection protein; that is, the antibodies show little or no cross-reactivity to other proteins, such as related family members. The Hepatitis C infection antibodies may be coupled to standard affinity chromatography columns and used to purify proteins associated with Hepatitis C infection. The antibodies may also be used as blocking polypeptides, as outlined above, since they will specifically bind to the Hepatitis C infection protein.

Methods of Identifying Variant Hepatitis C Infection-Associated Sequences

Without being bound by theory, expression of various Hepatitis C infection sequences is correlated with Hepatitis C infection. Accordingly, disorders based on mutant or variant Hepatitis C infection genes may be determined. In one embodiment, the invention provides methods for identifying cells containing variant Hepatitis C infection genes, e.g., determining all or part of the sequence of at least one endogenous Hepatitis C infection genes in a cell. This may be accomplished using many sequencing techniques. In a preferred embodiment, the invention provides methods of identifying the Hepatitis C infection genotype of an individual, e.g., determining all or part of the sequence of at least one Hepatitis C infection gene of the individual. This is generally done in at least one tissue of the individual, and may include the evaluation of a number of tissues or different samples of the same tissue. The method may include comparing the sequence of the sequenced Hepatitis C infection gene to a known Hepatitis C infection gene, e.g., a wild-type gene.

The sequence of all or part of a Hepatitis C infection gene can then be compared to the sequence of a known Hepatitis C infection gene to determine if any differences exist. This can be done using, e.g., known homology programs, such as Bestfit, etc. In a preferred embodiment, the presence of a difference in the sequence between the Hepatitis C infection gene of the patient and the known Hepatitis C infection gene correlates with a disease state or a propensity for a disease state, or susceptibility to effective treatment, as outlined herein.

In a preferred embodiment, the Hepatitis C infection genes are used as probes to determine the number of copies of the Hepatitis C infection gene in the genome.

In another preferred embodiment, the Hepatitis C infection genes are used as probes to determine the chromosomal localization of the Hepatitis C infection genes. Information such as chromosomal localization finds use in providing a diagnosis or prognosis in particular when chromosomal abnormalities such as translocations, and the like are identified in the Hepatitis C infection gene locus.

Administration of Pharmaceutical and Vaccine Compositions

In one embodiment, a therapeutically effective dose of a Hepatitis C infection protein or modulator thereof, is administered to a patient. By “therapeutically effective dose” herein is meant a dose that produces effects for which it is administered. The exact dose will depend on the purpose of the treatment, and will be ascertainable by one skilled in the art using known techniques. See, e.g., Ansel, et al. (1999) Pharmaceutical Dosage Forms and Drug Delivery Lippincott; Lieberman (1992) Pharmaceutical Dosage Forms (vols. 1-3) Dekker, ISBN 0824770846, 082476918X, 0824712692, 0824716981; Lloyd (1999) The Art, Science and Technology of Pharmaceutical Compounding Amer. Pharmaceut. Assn.; and Pickar (1998) Dosage Calculations Thomson. Adjustments for testicular cancer degradation, systemic versus localized delivery, and rate of new protease synthesis, as well as the age, body weight, general health, sex, diet, time of administration, drug interaction, and the severity of the condition may be necessary.

A “patient” for the purposes of the present invention includes both humans and other animals, particularly mammals. Thus the methods are applicable to both human therapy and veterinary applications. In the preferred embodiment the patient is a mammal, preferably a primate, and in the most preferred embodiment the patient is human.

The administration of the Hepatitis C infection proteins and modulators thereof of the present invention can be done in a variety of ways as discussed above, including, but not limited to, orally, subcutaneously, intravenously, intranasally, transdermally, intraperitoneally, intramuscularly, intrapulmonary, vaginally, rectally, or intraocularly. In some instances, e.g., in the treatment of wounds and inflammation, the Hepatitis C infection proteins and modulators may be directly applied as a solution or spray.

The pharmaceutical compositions of the present invention comprise a Hepatitis C infection protein in a form suitable for administration to a patient. In the preferred embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise unuseable, formed with inorganic acids such as hydrochloric acid, hydrobromic acid, sulfuric acid, nitric acid, phosphoric acid, and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. “Pharmaceutically acceptable base addition salts” include those derived from inorganic bases such as sodium, potassium, lithium, ammonium, calcium, magnesium, iron, zinc, copper, manganese, aluminum salts, and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers; fillers such as microcrystalline cellulose, lactose, corn and other starches; binding agents; sweeteners and other flavoring agents; coloring agents; and polyethylene glycol.

The pharmaceutical compositions can be administered in a variety of unit dosage forms depending upon the method of administration. For example, unit dosage forms suitable for oral administration include, but are not limited to, powder, tablets, pills, capsules and lozenges. It is recognized that Hepatitis C infection protein modulators (e.g., antibodies, antisense constructs, ribozymes, small organic molecules, etc.) when administered orally, should be protected from digestion. This is typically accomplished either by complexing the molecule(s) with a composition to render it resistant to acidic and enzymatic hydrolysis, or by packaging the molecule(s) in an appropriately resistant carrier, such as a liposome or a protection barrier. Means of protecting agents from digestion are available.

The compositions for administration will commonly comprise a Hepatitis C infection protein modulator dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like in accordance with the particular mode of administration selected and the patient's needs. See, e.g., (1980) Remington's Pharmaceutical Science (18th ed.) Mack; and Hardman and Limbird (eds. 2001) Goodman and Gilman: The Pharmacological Basis of Therapeutics (10th ed.) McGraw-Hill.

Thus, a typical pharmaceutical composition for intravenous administration would be about 0.1 to 10 mg per patient per day. Dosages from 0.1 up to about 100 mg per patient per day may be used, particularly when the drug is administered to a secluded site and not into the blood stream, such as into a body cavity or into a lumen of an organ. Substantially higher dosages are possible in topical administration. Actual methods for preparing parenterally administrable compositions will be known or apparent. See, e.g., Remington's Pharmaceutical Science and Goodman and Gilman: The Pharmacological Basis of Therapeutics, supra.

The compositions containing modulators of Hepatitis C infection proteins can be administered for therapeutic or prophylactic treatments. In therapeutic applications, compositions are administered to a patient suffering from infection in an amount sufficient to cure or at least partially arrest the disease and its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. The composition should provide a sufficient quantity of the agents of this invention to effectively treat the patient. An amount of modulator that is capable of preventing or slowing the development of disease progression in a mammal is referred to as a “prophylactically effective dose.” The particular dose required for a prophylactic treatment will depend upon the medical condition and history of the mammal, the particular strains being prevented, as well as other factors such as age, weight, gender, administration route, efficiency, etc. Such prophylactic treatments may be used, e.g., in a mammal who has previously had infection to prevent a recurrence of the infection, or in a mammal who is suspected of having a significant likelihood of developing progression. Vaccine strategies may be used, in either a DNA vaccine form, or protein vaccine.

It will be appreciated that the present Hepatitis C infection protein-modulating compounds can be administered alone or in combination with additional Hepatitis C infection modulating compounds or with other therapeutic agent, e.g., other anti-viral agents or treatments.

In numerous embodiments, one or more nucleic acids, e.g., polynucleotides comprising nucleic acid sequences set forth in Tables 1A-15, such as antisense polynucleotides or ribozymes, will be introduced into cells, in vitro or in vivo. The present invention provides methods, reagents, vectors, and cells useful for expression of Hepatitis C infection-associated polypeptides and nucleic acids using in vitro (cell-free), ex vivo or in vivo (cell or organism-based) recombinant expression systems.

The particular procedure used to introduce the nucleic acids into a host cell for expression of a protein or nucleic acid is application specific. Many procedures for introducing foreign nucleotide sequences into host cells may be used. These include the use of calcium phosphate transfection, spheroplasts, electroporation, liposomes, microinjection, plasma vectors, viral vectors and any of the other well known methods for introducing cloned genomic DNA, cDNA, synthetic DNA or other foreign genetic material into a host cell. See, e.g., Berger and Kimmel (1987) Guide to Molecular Cloning Techniques from Methods in Enzymology (vol. 152) Academic Press; Ausubel, et al. (eds. 1999 and supplements) Current Protocols Lippincott; and Sambrook, et al. (2001) Molecular Cloning: A Laboratory Manual (3d ed., Vol. 1-3) CSH Press.

In a preferred embodiment, Hepatitis C infection proteins and modulators are administered as therapeutic agents, and can be formulated as outlined above. Similarly, Hepatitis C infection genes (including both the full-length sequence, partial sequences, or regulatory sequences of the Hepatitis C infection coding regions) can be administered in a gene therapy application. These Hepatitis C infection genes can include inhibitory applications, e.g., inhibitory RNA, gene therapy (e.g., for incorporation into the genome), or antisense compositions.

Hepatitis C infection polypeptides and polynucleotides can also be administered as vaccine compositions to stimulate HTL, CTL, and antibody responses. Such vaccine compositions can include, e.g., lipidated peptides (see, e.g., Vitiello, et al. (1995) J. Clin. Invest. 95:341-349), peptide compositions encapsulated in poly(DL-lactide-co-glycolide) (“PLG”) microspheres (see, e.g., Eldridge, et al. (1991) Molec. Immunol. 28:287-294; Alonso, et al. (1994) Vaccine 12:299-306; Jones, et al. (1995) Vaccine 13:675-681), peptide compositions contained in immune stimulating complexes (ISCOMS) (see, e.g., Takahashi, et al. (1990) Nature 344:873-875; Hu, et al. (1998) Clin. Exp. Immunol. 113:235-243), multiple antigen peptide systems (MAPs) (see, e.g., Tam (1988) Proc. Nat'l Acad. Sci. USA 85:5409-5413; Tam (1996) J. Immunol. Meth. 196:17-32), peptides formulated as multivalent peptides; peptides for use in ballistic delivery systems, typically crystallized peptides, viral delivery vectors (Perkus, et al., p. 379, in Kaufmann (ed. 1996) Concepts in Vaccine Development de Gruyter; Chakrabarti, et al. (1986) Nature 320:535-537; Hu, et al. (1986) Nature 320:537-540; Kieny, et al. (1986) Bio/Technology 4:790-795; Top, et al. (1971) J. Infect. Dis. 124:148-154; Chanda, et al. (1990) Virology 175:535-547), particles of viral or synthetic origin (see, e.g., Kofler, et al. (1996) J. Immunol. Meth. 192:25-35; Eldridge, et al. (1993) Sem. Hematol. 30:16-24; Falo, et al. (1995) Nature Med. 1:649-653), adjuvants (Warren, et al. (1986) Ann. Rev. Immunol. 4:369-388; Gupta, et al. (1993) Vaccine 11:293-306), liposomes (Reddy, et al. (1992) J. Immunol. 148:1585-1589; Rock (1996) Immunol. Today 17:131-137), or naked or particle absorbed cDNA (Ulmer, et al. (1993) Science 259:1745-1749; Robinson, et al. (1993) Vaccine 11:957-960; Shiver, et al., p 423, in Kaufmann (ed. 1996) Concepts in Vaccine Development de Gruyter; Cease and Berzofsky (1994) Ann. Rev. Immunol. 12:923-989; and Eldridge, et al. (1993) Sem. Hematol. 30:16-24). Toxin-targeted delivery technologies, also known as receptor mediated targeting, such as those of Avant Immunotherapeutics, Inc. (Needham, Mass.) may also be used.

Vaccine compositions often include adjuvants. Many adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Certain adjuvants are commercially available as, e.g., Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, interleukin-2, -7, -12, and other like growth factors, may also be used as adjuvants.

Vaccines can be administered as nucleic acid compositions wherein DNA or RNA encoding one or more of the polypeptides, or a fragment thereof, is administered to a patient. This approach is described, for instance, in Wolff, et al., Science 247:1465 (1990) as well as U.S. Pat. Nos. 5,580,859; 5,589,466; 5,804,566; 5,739,118; 5,736,524; 5,679,647; WO 98/04720; and in more detail below. Examples of DNA-based delivery technologies include “naked DNA”, facilitated (bupivicaine, polymers, peptide-mediated) delivery, cationic lipid complexes, and particle-mediated (“gene gun”) or pressure-mediated delivery (see, e.g., U.S. Pat. No. 5,922,687).

For therapeutic or prophylactic immunization purposes, the peptides of the invention can be expressed by viral or bacterial vectors. Examples of expression vectors include attenuated viral hosts, such as vaccinia or fowlpox. This approach involves the use of vaccinia virus, e.g., as a vector to express nucleotide sequences that encode Hepatitis C infection polypeptides or polypeptide fragments. Upon introduction into a host, the recombinant vaccinia virus expresses the immunogenic peptide, and thereby elicits an immune response. Vaccinia vectors and methods useful in immunization protocols are described in, e.g., U.S. Pat. No. 4,722,848. Another vector is BCG (Bacille Calmette Guerin). See Stover, et al. (1991) Nature 351:456-460. A wide variety of other vectors are available for therapeutic administration or immunization, e.g., adeno and adeno-associated virus vectors, retroviral vectors, Salmonella typhi vectors, detoxified anthrax toxin vectors, and the like. See, e.g., Shata, et al. (2000) Mol. Med. Today 6:66-71; Shedlock, et al. (2000) J. Leukoc. Biol. 68:793-806; and Hipp, et al. (2000) In Vivo 14:571-85).

Methods for the use of genes as DNA vaccines are well known, and include placing a Hepatitis C infection gene or portion of a Hepatitis C infection gene under the control of a regulatable promoter or a tissue-specific promoter for expression in a Hepatitis C infection patient. The Hepatitis C infection gene used for DNA vaccines can encode full-length Hepatitis C infection proteins, but more preferably encodes portions of the Hepatitis C infection proteins including peptides derived from the Hepatitis C infection protein. In one embodiment, a patient is immunized with a DNA vaccine comprising a plurality of nucleotide sequences derived from a Hepatitis C infection gene. For example, Hepatitis C infection-associated genes or sequence encoding subfragments of a Hepatitis C infection protein are introduced into expression vectors and tested for their immunogenicity in the context of Class I MHC and an ability to generate cytotoxic T cell responses. This procedure provides for production of cytotoxic T cell responses against cells which present antigen, including intracellular epitopes.

In a preferred embodiment, the DNA vaccines include a gene encoding an adjuvant or accessory molecule with the DNA vaccine. Such adjuvant molecules may include cytokines that increase the immunogenic response to the Hepatitis C infection polypeptide encoded by the DNA vaccine. Additional or alternative adjuvants are available.

In another preferred embodiment Hepatitis C infection genes find use in generating animal models of Hepatitis C infection. When the Hepatitis C infection gene identified is repressed or diminished in cancer tissue, gene therapy technology, e.g., wherein antisense RNA directed to the Hepatitis C infection gene will also diminish or repress expression of the gene. Animal models of Hepatitis C infection find use in screening for modulators of a Hepatitis C infection-associated sequence or modulators of Hepatitis C infection. Similarly, transgenic animal technology including gene knockout technology, e.g., as a result of homologous recombination with an appropriate gene targeting vector, will result in the absence or increased expression of the Hepatitis C infection protein. When desired, tissue-specific expression or knockout of the Hepatitis C infection protein may be necessary.

It is also possible that the Hepatitis C infection protein is overexpressed during Hepatitis C infection. As such, transgenic animals can be generated that overexpress the Hepatitis C infection protein. Depending on the desired expression level, promoters of various strengths can be employed to express the transgene. Also, the number of copies of the integrated transgene can be determined and compared for a determination of the expression level of the transgene. Animals generated by such methods find use as animal models of Hepatitis C infection and are additionally useful in screening for modulators to treat Hepatitis C infection and/or its secondary consequences.

Kits for Use in Diagnostic and/or Prognostic Applications

For use in diagnostic, research, and therapeutic applications suggested above, kits are also provided by the invention. In the diagnostic and research applications such kits may include some of the following: assay reagents, buffers, Hepatitis C infection-specific nucleic acids or antibodies, hybridization probes and/or primers, antisense polynucleotides, ribozymes, dominant negative Hepatitis C infection polypeptides or polynucleotides, small molecules inhibitors of Hepatitis C infection-associated sequences etc. A therapeutic product may include sterile saline or another pharmaceutically acceptable emulsion and suspension base.

In addition, the kits may include instructional materials containing directions (e.g., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

The present invention also provides for kits for screening for modulators of Hepatitis C infection-associated sequences. Such kits can be prepared from readily available materials and reagents. For example, such kits can comprise one or more of the following materials: a Hepatitis C infection-associated polypeptide or polynucleotide, reaction tubes, and instructions for testing Hepatitis C infection-associated activity. Optionally, the kit contains biologically active Hepatitis C infection protein. A wide variety of kits and components can be prepared according to the present invention, depending upon the intended user of the kit and the particular needs of the user. Diagnosis would typically involve evaluation of a plurality of genes or products. The genes will be selected based on correlations with important parameters in disease which may be identified in historical or outcome data.

It is understood that the examples described above in no way serve to limit the true scope of this invention, but rather are presented for illustrative purposes. All publications, sequences of accession numbers, and patent applications cited in this specification are herein incorporated by reference as if each individual publication or patent application were specifically and individually indicated to be incorporated by reference.

### EXAMPLES

### Example 1

### Gene Chip Analyses

Molecular profiles of various normal and testicular cancer tissues were determined and analyzed using gene chips. RNA was isolated and gene chip analysis was performed as described (Glynne, et al. (2000) Nature 403:672-676; Zhao, et al. (2000) Genes Dev. 14:981-993).

Tables 1B-14B list the accession numbers for those Pkey's lacking UnigeneID's for tables 1A-14A. For each probeset is listed the gene cluster number from which nucleotides were designed. Gene clusters were compiled using sequences derived from Genbank ESTs and mRNAs. These sequences were clustered based on sequence similarity using Clustering and Alignment Tools (DoubleTwist, Oakland Calif.). Genbank accession numbers for sequences comprising each cluster are listed in the “Accession” column.

Tables 1C-14C list genomic positioning for Pkeys lacking Unigene ID's and accession numbers in tables 1A-14A. For each predicted exon is listed genomic sequence source used for prediction. Nucleotide locations of each predicted exon are also listed.

TABLE 1A
589 GENES UPREGULATED IN HEPATITIS C [see 60/308, 188]

| Pkey | ExAccn | UnigeneID | Title | R1 |
|---|---|---|---|---|
| 428227 | AA321649 | Hs. 2248 | INTERFERON-GAMMA INDUCED PROTEIN PRECURSOR (G | 32 |
| 426711 | AA383471 | Hs. 180669 | conserved gene amplified in osteosarcoma | 20 |
| 408063 | BE086548 | Hs. 42346 | calcineurin-binding protein calsarcin-1 | 19 |
| 422746 | NM_004484 | Hs. 119651 | glypican 3 | 18 |
| 418318 | U47732 | Hs. 84072 | transmembrane 4 superfamily member 3 | 11 |
| 414052 | AW578849 | Hs. 283552 | ESTs, Weakly similar to unnamed protein produ | 11 |
| 453319 | AI985369 | Hs. 20117 | ESTs | 10 |
| 424878 | H57111 | Hs. 221132 | ESTs | 10 |
| 426793 | X89887 | Hs. 172350 | HIR (histone cell cycle regulation defective, | 10 |
| 434210 | AA665612 | Hs. 120874 | ESTs | 9 |
| 449613 | N63808 | Hs. 34299 | ESTs | 8 |
| 421904 | BE143533 | Hs. 109309 | hypothetical protein FLJ20035 | 8 |
| 427283 | AL119796 | Hs. 174185 | ectonucleotide pyrophosphatase/phosphodiester | 8 |
| 416206 | AW206248 | Hs. 111092 | Homo sapiens cDNA: FLJ22332 fis, clone HRC057 | 8 |
| 408096 | BE250162 | Hs. 83765 | dihydrofolate reductase | 8 |
| 447541 | AK000288 | Hs. 18800 | hypothetical protein FLJ20281 | 8 |
| 414812 | X72755 | Hs. 77367 | monokine induced by gamma interferon | 8 |
| 436169 | AA888311 | Hs. 17602 | Homo sapiens cDNA FLJ12381 fis, clone MAMMA10 | 7 |
| 409231 | AA446644 | Hs. 692 | GA733-2; epithelial glycoprotein (EGP) (KSA) | 7 |
| 418216 | AA662240 | Hs. 283099 | AF15q14 protein | 7 |
| 432094 | AI658580 | Hs. 61426 | ESTs | 7 |
| 425053 | AF046024 | Hs. 154320 | ubiquitin-activating enzyme E1C (homologous t | 7 |
| 417621 | AV654694 | Hs. 82316 | interferon-induced, hepatitis C-associated mi | 7 |
| 414004 | AA737033 | Hs. 7155 | ESTs, Weakly similar to 2115357A TYKi protein | 7 |
| 426052 | N49068 | Hs. 93966 | ESTs | 7 |
| 435102 | AW899053 | Hs. 76917 | F-box only protein 8 | 7 |
| 417788 | AI436699 | Hs. 84928 | nuclear transcription factor Y, beta | 7 |
| 445757 | AW449065 | Hs. 13264 | KIAA0856 protein | 7 |
| 456619 | AV647917 | Hs. 107153 | inhibitor of growth family, member 1-like | 7 |
| 419743 | AW408762 | Hs. 127478 | ESTs | 7 |
| 414737 | AI160386 | Hs. 125087 | ESTs | 7 |
| 428708 | NM_014897 | Hs. 190386 | KIAA0924 protein | 7 |
| 449718 | AA459480 | Hs23956 | hypothetical protein FLJ20502 | 6 |
| 448402 | BE244226 | Hs. 21094 | RAB18, member RAS oncogene family | 6 |
| 434733 | AI334367 | Hs. 159337 | ESTs | 6 |
| 425266 | J00077 | Hs. 155421 | alpha-fetoprotein | 6 |
| 432706 | NM_013230 | Hs. 286124 | CD24 | 6 |
| 407204 | R41933 | Hs. 140237 | ESTs, Weakly similar to AF119917 13 PRO1722 [ | 6 |
| 409401 | AI201895 | Hs. 181309 | proteasome (prosome, macropain) subunit, alph | 6 |
| 424626 | AA344308 | Hs. 128427 | ESTs | 6 |
| 443547 | AW271273 | Hs. 23767 | ESTs | 6 |
| 409068 | AW236991 | Hs. 102495 | ESTs | 6 |
| 422173 | BE385828 | Hs. 250619 | phorbolin-like protein MDS019 | 6 |
| 425815 | R94023 | Hs. 94560 | ESTs | 6 |
| 448111 | AA053486 | Hs. 20315 | interferon-induced protein with tetratricopep | 6 |
| 430594 | AK000790 | Hs. 246885 | hypothetical protein FLJ20783 | 6 |
| 432435 | BE218886 | Hs. 282070 | ESTs | 6 |
| 449245 | AI636539 | Hs. 224296 | ESTs | 6 |
| 400719 | NM_004055 | Hs. 6133 | calpain 5 | 6 |
| 442961 | BE614474 | Hs. 289074 | Homo sapiens cDNA FLJ13986 fis, clone Y79AA10 | 6 |
| 409153 | W03754 | Hs. 50813 | hypothetical protein FLJ20022 | 6 |
| 422553 | AI697720 | Hs. 171455 | ESTs | 6 |
| 442432 | BE093589 | Hs. 38178 | Homo sapiens cDNA: FLJ23468 fis, clone HSI116 | 6 |
| 437086 | AW291411 | Hs. 192531 | ESTs, Weakly similar to AF244088 1 zinc finge | 6 |
| 401598 | AA172106 | Hs. 110950 | Rag C protein | 6 |
| 438523 | H66220 | Hs. 278177 | ESTs | 6 |
| 437267 | AW511443 | Hs. 258110 | ESTs | 6 |
| 408035 | NM_006242 | Hs. 42215 | protein phosphatase 1, regulatory subunit 6 | 5 |
| 433401 | AF039698 | Hs. 284217 | serologically defined colon cancer antigen 33 | 5 |
| 410541 | AA065003 | Hs. 64179 | hypothetical protein | 5 |
| 455743 | BE073754 | | gb: RC0-BT0561-210100-032-d07 BT0561 Homo sapi | 5 |
| 429167 | BE465867 | Hs. 197751 | KIAA0666 protein | 5 |
| 429490 | AI971131 | Hs. 293684 | ESTs, Weakly similar to alternatively spliced | 5 |
| 434263 | N34895 | Hs. 44648 | ESTs | 5 |
| 441046 | W01538 | Hs. 126742 | ESTs | 5 |
| 452327 | AK000196 | Hs. 29052 | hypothetical protein FLJ20189 | 5 |
| 416504 | T85831 | Hs. 16004 | ESTs | 5 |
| 432723 | D29677 | Hs. 3085 | KIAA0054 gene product; Helicase | 5 |
| 423588 | W18186 | Hs. 117688 | ESTs, Weakly similar to ALU5_HUMAN ALU SUBFAM | |
| 432960 | AW150945 | Hs. 144758 | ESTs | 5 |
| 403041 | c21p3_565 | predicted exon | | |
| 433001 | AF217513 | Hs. 279905 | clone HQ0310 PRO0310p1 | 5 |
| 419407 | AW410377 | Hs. 41502 | Homo sapiens cDNA: FLJ21276 fis, clone COL018 | 5 |
| 411252 | AB018549 | Hs. 69328 | MD-2 protein | 5 |
| 444670 | H58373 | Hs. 37494 | ESTs | 5 |
| 445525 | BE149866 | Hs. 14831 | ESTs | 5 |
| 414646 | AA353776 | Hs. 901 | CD48 antigen (B-cell membrane protein) | 5 |
| 429747 | M87507 | Hs. 2490 | caspase 1, apoptosis-related cysteine proteus | 5 |
| 434666 | AF151103 | Hs. 112259 | T cell receptor gamma locus | 5 |
| 413541 | BE147036 | | gb: QV4-HT0222-091199-024-e10 HT0222 Homo sapi | 5 |
| 424737 | BE301883 | Hs. 152707 | glioblastoma amplified sequence | 5 |
| 426780 | BE242284 | Hs. 172199 | adenylate cyclase 7 | 5 |
| 441562 | AW578981 | Hs. 52184 | hypothetical protein FLJ20618 | 5 |
| 446459 | AI680731 | Hs. 170399 | ESTs | 5 |
| 409342 | AU077058 | Hs. 54089 | BRCA1 associated RING domain 1 | 5 |
| 410577 | X91911 | Hs. 64639 | glioma pathogenesis-related protein | 5 |
| 428250 | AW809208 | Hs. 183297 | DKFZP566F2124 protein | 5 |
| 450447 | AF212223 | Hs. 25010 | hypothetical protein P15-2 | 5 |
| 430261 | AA305127 | Hs. 237225 | ribosomal protein S5 pseudogene 1 | 5 |
| 433312 | AI241331 | Hs. 131765 | ESTs | 5 |
| 442366 | AA115629 | Hs. 118531 | ESTs | 5 |
| 427846 | AW499770 | Hs. 180948 | KIAA0729 protein | 5 |
| 454075 | R43826 | Hs. 16313 | ESTs | 5 |
| 425324 | M89470 | Hs. 155644 | paired box gene 2 | 5 |
| 410527 | AW851066 | | gb: IL3-CT0220-150200-070-B02 CT0220 Homo sapi | 5 |
| 456439 | AA251242 | Hs. 103238 | ESTs | 5 |
| 434948 | AI498469 | Hs. 12622 | ESTs, Highly similar to AF161436 1 HSPC318 [H | 5 |
| 436488 | BE620909 | Hs. 261023 | hypothetical protein FLJ20958 | 5 |
| 411466 | AW847669 | | gb: IL3-CT0213-280100-056-G10 CT0213 Homo sapi | 5 |
| 432378 | AI493046 | Hs. 146133 | ESTs | 5 |
| 402526 | c1p1_17942 | predicted exon | | |
| 420433 | NM_007016 | Hs. 97627 | protein similar to E.coli yhdg and R. capsula | 5 |
| 441595 | AW206035 | Hs. 192123 | ESTs | 5 |
| 430008 | AW085625 | Hs. 186838 | ESTs; Weakly similar to similar to zinc finge | 5 |
| 434924 | AA443164 | Hs. 23259 | hypothetical protein FLJ13433 | 5 |
| 419003 | T78640 | Hs. 268595 | ESTs | 5 |
| 435126 | AI393666 | Hs. 42315 | Homo sapiens cDNA FLJ13036 fis, clone NT2RP30 | 5 |
| 447513 | AW955776 | | gb: EST367846 MAGE resequences, MAGD Homo sapi | 5 |
| 423258 | L13460 | Hs. 1644 | cytochrome P450, subfamily VIIA (cholesterol | 5 |
| 417308 | H60720 | Hs. 81892 | KIAA0101 gene product | 4 |
| 442760 | BE075297 | Hs. 10067 | ESTs, Weakly similar to WASP-family protein | 4 |
| 444758 | AL044878 | Hs. 11899 | 3-hydroxy-3-methylglutaryl-Coenzyme A reducta | 4 |
| 448752 | AA593867 | Hs. 170890 | Homo sapiens cDNA: FLJ21129 fis, clone CAS062 | 4 |
| 424623 | AW963062 | Hs. 165809 | ESTs | 4 |
| 431982 | AW419296 | Hs. 105754 | ESTs | 4 |
| 441028 | AI333660 | Hs. 17558 | ESTs | 4 |
| 402811 | c1p3_2372 | | predicted exon | 4 |
| 416839 | H94900 | Hs. 17882 | ESTs | 4 |
| 421029 | AW057782 | Hs. 293053 | ESTs | 4 |
| 417301 | AI478158 | Hs. 164478 | hypothetical protein FLJ21939 similar to 5-az | 4 |
| 430778 | D90337 | Hs. 247916 | natriuretic peptide precursor C | 4 |
| 443380 | AI792478 | Hs. 135377 | ESTs | 4 |
| 447183 | AI554733 | Hs. 173182 | ESTs | 4 |
| 456383 | AI148037 | | gb: qg61e01.r1 Soares_testis_NHT Homo sapiens | 4 |
| 458239 | BE439877 | Hs. 283389 | ESTs | 4 |
| 407190 | AA600135 | | gb: ae50c06.s1 Stratagene lung carcinoma 93721 | 4 |
| 425583 | AF077346 | Hs. 158315 | interleukin 18 receptor accessory protein | 4 |
| 439593 | BE073597 | Hs. 124863 | ESTs | 4 |
| 430200 | BE613337 | Hs. 234896 | geminin | 4 |
| 456299 | R93374 | Hs. 14173 | ESTs | 4 |
| 421939 | BE169531 | Hs. 109727 | TAK1-binding protein 2; KIAA0733 protein | 4 |
| 437282 | AI810593 | Hs. 16587 | ESTs | 4 |
| 410315 | AI638871 | Hs. 17625 | ESTs | 4 |
| 408380 | AF123050 | Hs. 44532 | diubiquitin | 4 |
| 442993 | BE018682 | Hs. 44343 | ESTs | 4 |
| 450560 | BE383204 | | gb: 601298758F1 NIH_MGC_19 Homo sapiens cDNA c | 4 |
| 407444 | AF229803 | | gb: Homo sapiens endozepine-like protein type | 4 |
| 408558 | AW015759 | Hs. 235709 | ESTs | 4 |
| 431214 | AA294921 | Hs. 250811 | v-ral simian leukemia viral oncogene homolog | 4 |
| 441887 | AW967865 | Hs. 92145 | ESTs | 4 |
| 413871 | W17187 | Hs. 75598 | heterogeneous nuclear ribonucleoprotein A2/B1 | 4 |
| 422363 | T55979 | Hs. 115474 | replication factor C (activator 1) 3 (38 kD) | 4 |
| 439175 | AF086021 | Hs. 271113 | ESTs | 4 |
| 419375 | W27916 | | gb: 39f6 Humun retina cDNA randomly primed sub | 4 |
| 424430 | AI769467 | Hs. 96769 | ESTs | 4 |
| 435571 | AF212225 | Hs. 283693 | BM022 protein | 4 |
| 425100 | AF051850 | Hs. 154567 | supervillin | 4 |
| 410430 | AW732554 | | gb: bb08b10.y1 NIH_MGC_14 Homo sapiens cDNA cl | 4 |
| 430273 | AI311127 | Hs. 125522 | ESTs | 4 |
| 449052 | AW029507 | Hs. 161102 | ESTs | 4 |
| 454750 | AW866285 | | gb: QV4-SN0024-080400-167-a09 SN0024 Homo sapi | 4 |
| 429732 | U20158 | Hs. 2488 | lymphocyte cytosolic protein 2 (SH2 domain-co | 4 |
| 403747 | c4p1_4301 | | predicted exon | 4 |
| 418724 | AA460597 | Hs. 87784 | hypothetical protein dJ102H19.4 | 4 |
| 414899 | AW975433 | Hs. 36288 | ESTs | 4 |
| 433112 | AA973801 | Hs. 144553 | ESTs, Weakly similar to unnamed protein produ | 4 |
| 439971 | W32474 | Hs. 7942 | hypothetical protein FLJ20080 | 4 |
| 421170 | BE217797 | Hs. 126052 | ESTs | 4 |
| 443454 | AI057494 | Hs. 133421 | ESTs | 4 |
| 443885 | H91806 | Hs. 15284 | ESTs | 4 |
| 446648 | AL137521 | Hs. 15797 | Homo sapiens mRNA; cDNA DKFZp434D0218 (from c | 4 |
| 456182 | H85328 | Hs. 239045 | ESTs | 4 |
| 431318 | AA502700 | Hs. 293147 | ESTs | 4 |
| 453950 | AA156998 | Hs. 211568 | eukaryotic translation initiation factor 4 ga | 4 |
| 411857 | AW879403 | | gb: PM0-OT0019-150300-002-d01 OT0019 Homo sapi | 4 |
| 409205 | AI952884 | Hs. 14832 | ESTs, Moderately similar to unnamed protein p | 4 |
| 411083 | N41340 | Hs. 68318 | hypothetical protein FLJ20344 | 4 |
| 428035 | AA482027 | Hs. 142569 | ESTs | 4 |
| 435703 | AW630133 | Hs. 83313 | GK003 protein | 4 |
| 413786 | AW613780 | Hs. 13500 | ESTs | 4 |
| 434375 | BE277910 | Hs. 3833 | 3′-phosphoadenosine 5′-phosphosulfate synthas | 4 |
| 448478 | AI523218 | Hs. 203456 | ESTs | 4 |
| 451192 | AA019551 | Hs. 60687 | ESTs, Moderately similar to KIAA0544 protein | 4 |
| 457235 | L20433 | Hs. 211588 | POU domain, class 4, transcription factor 1 | 4 |
| 425491 | AA883316 | Hs. 255221 | ESTs | 4 |
| 430820 | AF194815 | Hs. 248012 | immunoglobulin lambda variable 4-3 | 4 |
| 432572 | AI660840 | Hs. 191202 | ESTs, Weakly similar to ALUE_HUMAN !!!! ALU C | 4 |
| 457250 | AA811987 | Hs. 125779 | ESTs | 4 |
| 406367 | ph2_15338 | | predicted exon | 4 |
| 455960 | BE165256 | | gb: QV1-HT0473-130300-106-f04 HT0473 Homo sapi | 4 |
| 429503 | AA394183 | Hs. 26873 | ESTs | 4 |
| 419286 | AA236005 | Hs. 221303 | ESTs | 4 |
| 453555 | N23574 | Hs. 123649 | ESTs, Moderately similar to ALU7_HUMAN ALU SU | 4 |
| 423165 | AI937547 | Hs. 124915 | Human DNA sequence from clone 380A1 on chromo | 4 |
| 402230 | c19p1_5271 | predicted exon | DAL8 | |
| 451356 | AA748418 | Hs. 164577 | ESTs | 4 |
| 409712 | AA167385 | Hs. 13583 | ESTs | 4 |
| 442281 | N34742 | Hs. 170065 | Homo sapiens cDNA FLJ13492 fis, clone PLACE10 | 4 |
| 434941 | AW073202 | Hs. 18368 | DKFZP564B0769 protein | 4 |
| 424145 | AW802763 | Hs. 193124 | ESTs | 4 |
| 430665 | BE350122 | Hs. 157367 | ESTs | 4 |
| 442878 | AI868648 | Hs. 22315 | ESTs | 4 |
| 457307 | AI311127 | Hs. 125522 | ESTs | 4 |
| 414522 | AW518944 | Hs. 76325 | Homo sapiens cDNA: FLJ23125 fis, clone LNG082 | 4 |
| 421650 | AA781795 | Hs. 122587 | ESTs | 4 |
| 426416 | AW612744 | Hs. 169824 | killer cell lectin-like receptor subfamily B, | 4 |
| 458836 | AI568607 | Hs. 182112 | ESTs | 4 |
| 424113 | AI743880 | Hs. 12876 | ESTs | 4 |
| 418549 | AA927177 | Hs. 86041 | CGG triplet repeat binding protein 1 | 4 |
| 442297 | NM_006202 | Hs. 89901 | phosphodiesterase 4A, cAMP-specific (dunce (D | 4 |
| 419046 | T81816 | Hs. 193723 | ESTs | 4 |
| 429025 | AI399910 | Hs. 4842 | ESTs | 4 |
| 433230 | AW136134 | Hs. 220277 | ESTs | 4 |
| 452744 | AI267652 | Hs. 30504 | Homo sapiens mRNA; cDNA DKFZp434E082 (from cl | 4 |
| 446488 | AB037782 | Hs. 15119 | KIAA1361 protein | 4 |
| 422052 | AA302744 | Hs. 104518 | ESTs | 4 |
| 430299 | W28673 | Hs. 106747 | serine carboxypeptidase 1 precursor protein | 4 |
| 442431 | BE349856 | | gb: ht05a12.x1 NCI_CGAP_Kid13 Homo sapiens cDN | 4 |
| 411653 | AF070578 | Hs. 71168 | Homo sapiens clone 24674 mRNA sequence | 4 |
| 440384 | AA884275 | Hs. 137052 | ESTs | 4 |
| 443542 | AI927065 | Hs. 146040 | ESTs | 4 |
| 443638 | AW028696 | Hs. 145679 | ESTs | 4 |
| 431910 | AK000142 | Hs. 272192 | Homo sapiens mRNA tor KIAA1590 protein, parti | 4 |
| 440381 | AA917808 | Hs. 190495 | ESTs | 4 |
| 407272 | X98958 | | gb: H. sapiens rearranged Ig heavy chain (clone | 4 |
| 417173 | U61397 | Hs. 81424 | ubiquitin-like 1 (sentrin) | 4 |
| 417675 | AI808607 | Hs. 3781 | similar to murine leucine-rich repeat protein | 4 |
| 446552 | AW470827 | Hs. 156241 | ESTs | 4 |
| 450697 | AW152166 | Hs. 182113 | ESTs | 4 |
| 411960 | R77776 | Hs. 18103 | ESTs | 4 |
| 418637 | T86737 | Hs. 193536 | ESTs | 4 |
| 444065 | AW449415 | Hs. 10260 | Homo sapiens cDNA FLJ11341 fis, clone PLACE10 | 4 |
| 409038 | T97490 | Hs. 50002 | small inducible cytokine subfamily A (Cys-Cys | 3 |
| 418205 | L21715 | Hs. 83760 | troponin I, skeletal, fast | 3 |
| 435177 | AI018174 | Hs. 42936 | ESTs | 3 |
| 436027 | AI864053 | Hs. 39972 | ESTs, Weakly similar to I38588 reverse transc | 3 |
| 452420 | BE564871 | Hs. 29463 | centrin, EF-hand protein, 3 (CDC31 yeast homo | 3 |
| 423839 | AA985505 | Hs. 127217 | ESTs | 3 |
| 435163 | AA668884 | Hs. 19155 | ESTs | 3 |
| 449704 | AK000733 | Hs. 23900 | GTPase activating protein | 3 |
| 433854 | AA610649 | | gb: np95c03.s1 NCI_CGAP_Thy1 Homo sapiens cDNA | 3 |
| 414821 | M63835 | Hs. 77424 | Fc fragment of IgG, high affinity Ia, recepto | 3 |
| 431300 | AA502346 | | gb: ne26b03.s1 NCI_CGAP_Co3 Homo sapiens cDNA | 3 |
| 407469 | D55640 | | gb: Human monocyte PABL (pseudoautosomal bound | 3 |
| 450515 | AW304226 | Hs. 7298 | biphenyl hydrolase-like (serine hydrolase; br | 3 |
| 430750 | AI650360 | Hs. 100256 | ESTs | 3 |
| 410833 | AW806900 | | gb: QV4-ST0023-160400-172-d02 ST0023 Homo sapi | 3 |
| 426062 | N57014 | Hs. 44013 | ESTs | 3 |
| 432945 | AL043683 | Hs. 271357 | ESTs, Weakly similar to unnamed protein produ | 3 |
| 455708 | BE069326 | | gb: QV3-BT0381-170100-060-g03 BT0381 Homo sapi | 3 |
| 443240 | R02419 | Hs. 15338 | ESTs | 3 |
| 451429 | AA525993 | Hs. 173699 | ESTs, Weakly similar to ALU1_HUMAN ALU SUBFAM | 3 |
| 435812 | AA700439 | Hs. 188490 | ESTs | 3 |
| 453799 | N32080 | Hs. 271700 | ESTs | 3 |
| 406970 | M29994 | | gb: Human alpha-I spectrin gene, exon 12. | 3 |
| 425195 | AA352026 | | gb: EST59954 Infant brain Homo sapiens cDNA 5′ | 3 |
| 428180 | AI129767 | Hs. 182874 | Homo sapiens cDNA: FLJ21929 fis, clone HEP042 | 3 |
| 430753 | AI432401 | Hs. 2659 | fibrinogen-like 2 | 3 |
| 445800 | AA126419 | Hs. 301632 | ESTs | 3 |
| 439680 | AW245741 | Hs. 58461 | ESTs, Weakly similar to Unknown gene product | 3 |
| 444858 | AI199738 | Hs. 208275 | ESTs, Weakly similar to unnamed protein produ | 3 |
| 422879 | AI241409 | Hs. 188092 | ESTs | 3 |
| 444888 | AI651039 | Hs. 148559 | ESTs | 3 |
| 407897 | AA812234 | Hs. 270134 | hypothetical protein FLJ20280 | 3 |
| 409512 | AW979187 | Hs. 293591 | ESTs, Weakly similar to hypothetical protein | 3 |
| 419843 | AA749220 | Hs. 177708 | ESTs | 3 |
| 436053 | AI057224 | Hs. 15443 | ESTs | 3 |
| 420968 | AW968775 | Hs. 259760 | ESTs | 3 |
| 457707 | AW974642 | | gb: EST386746 MAGE resequences, MAGM Homo sapi | 3 |
| 442679 | R53718 | Hs. 107882 | hypothetical protein FLJ10659 | 3 |
| 434016 | AF113698 | Hs. 283774 | clone FLB7343 | 3 |
| 450330 | AW500775 | Hs. 24817 | hypothetical protein FLJ20136 | 3 |
| 454271 | AW293271 | Hs. 255179 | ESTs | 3 |
| 430382 | AA477908 | Hs. 282267 | ESTs | 3 |
| 458389 | H70284 | Hs. 160152 | ESTs, Weakly similar to FETA_HUMAN ALPHA-FETO | 3 |
| 413670 | AB000115 | Hs. 75470 | hypothetical protein, expressed in osteoblast | 3 |
| 451253 | H48299 | Hs. 26126 | claudin 10 | 3 |
| 442805 | AI201229 | Hs. 131262 | ESTs | 3 |
| 436260 | BE172762 | Hs. 292710 | ESTs, Weakly similar to ALU5_HUMAN ALU SUBFAM | 3 |
| 410099 | AA081630 | Hs. 169387 | KIAA0036 gene product | 3 |
| 430552 | AA176374 | Hs. 243886 | nuclear autoantigenic sperm protein (histone- | 3 |
| 452548 | AL050321 | Hs. 29846 | Human DNA sequence from clone 717M23 on chrom | 3 |
| 418183 | NM_001772 | Hs. 83731 | CD33 antigen (gp67) | 3 |
| 457470 | AB040973 | Hs. 272385 | G protein-coupled receptor 72 | 3 |
| 419135 | R61448 | Hs. 106728 | ESTs, Weakly similar to KIAA1353 protein [H.s | 3 |
| 448219 | AA228092 | Hs. 119569 | ESTs, Weakly similar to growth factor recepto | 3 |
| 413605 | BE152644 | | gb: CM1-HT0329-250200-128-f09 HT0329 Homo sapi | 3 |
| 435106 | AA100847 | Hs. 193380 | ESTs, Highly similar to AF174600 1 F-box prot | 3 |
| 410850 | AW362867 | Hs. 288699 | Homo sapiens cDNA: FLJ21425 fis, clone COL041 | 3 |
| 444665 | BE613126 | Hs. 47783 | ESTs, Weakly similar to T12540 hypothetical p | 3 |
| 454000 | AA040620 | Hs. 109144 | ESTs | 3 |
| 451644 | N23235 | Hs. 30567 | ESTs | 3 |
| 445594 | AW058463 | Hs. 12940 | zinc-fingers and homeoboxes 1 | 3 |
| 407696 | AI697340 | Hs. 76549 | ATPase, Na+/K+ transporting, alpha 1 polypept | 3 |
| 432606 | NM_002104 | Hs. 3066 | granzyme K (serine protease, granzyme 3; tryp | 3 |
| 435511 | AA683336 | Hs. 189046 | ESTs | 3 |
| 410324 | AW292539 | Hs. 30177 | ESTs | 3 |
| 429556 | AW139399 | Hs. 98988 | ESTs | 3 |
| 446751 | AA766998 | Hs. 85874 | ESTs, Weakly similar to predicted using Genef | 3 |
| 406038 | Y14443 | Hs. 88219 | zinc finger protein 200 | 3 |
| 440621 | AW296024 | Hs. 150434 | ESTs | 3 |
| 444342 | NM_014398 | Hs. 10887 | similar to lysosome-associated membrane glyco | 3 |
| 445715 | AB012958 | Hs. 13137 | UV radiation resistance associated gene | 3 |
| 417933 | X02308 | Hs. 82962 | thymidylate synthetase | 3 |
| 430929 | AA489166 | Hs. 156933 | ESTs | 3 |
| 400617 | AF151064 | Hs. 36069 | hypothetical protein | 3 |
| 402643 | c1p1_28109 | predicted exon | | |
| 410173 | AA706017 | Hs. 119944 | ESTs | 3 |
| 445664 | AW968638 | Hs. 237691 | ESTs | 3 |
| 437830 | AB020658 | Hs. 5867 | KIAA0851 protein | 3 |
| 404287 | c6p3_5616 | | predicted exon | 3 |
| 407366 | AF026942 | | gb: Homo sapiens cig33 mRNA, partial sequence. | 3 |
| 417771 | AA804698 | Hs. 82547 | retinoic acid receptor responder (tazarotene | 3 |
| 447645 | AW897321 | Hs. 159699 | ESTs | 3 |
| 454038 | X06374 | Hs. 37040 | platelet-derived growth factor alpha polypept | 3 |
| 442316 | Z75331 | Hs. 8217 | Homo sapiens cDNA: FLJ23025 fis, clone LNG017 | 3 |
| 433586 | T85301 | | gb: yd78d06.s1 Soares fetal liver spleen 1NFLS | 3 |
| 408622 | AA056060 | Hs. 202577 | EST cluster (not in UniGene) | 3 |
| 428249 | AA130914 | Hs. 183291 | zinc finger protein 268 | 3 |
| 448019 | AW947164 | Hs. 195641 | ESTs | 3 |
| 417228 | AL134324 | Hs. 7312 | ESTs | 3 |
| 430219 | X99209 | Hs. 235887 | HMT1 (hnRNP methyltransferase, S. cerevisiae) | 3 |
| 411060 | NM_006074 | Hs. 295978 | stimulated trans-acting factor (50 kDa) | 3 |
| 406805 | AI686003 | Hs. 296031 | ESTs | 3 |
| 426159 | Z44524 | Hs. 167456 | Homo sapiens mRNA full length insert cDNA clo | 3 |
| 432134 | AI816782 | Hs. 122583 | Homo sapiens cDNA: FLJ21934 fis, clone HEP043 | 3 |
| 454314 | AW364844 | | gb: QV3-DT0044-221299-045-c03 DT0044 Homo sapi | 3 |
| 441975 | AW173248 | Hs. 250139 | ESTs | 3 |
| 450937 | R49131 | Hs. 26267 | ATP-dependant interferon response protein 1 | 3 |
| 433529 | AA598547 | Hs. 222405 | ESTs | 3 |
| 454024 | AA993527 | Hs. 16281 | hypothetical protein FLJ23403 | 3 |
| 459352 | AW810383 | Hs. 206828 | ESTs | 3 |
| 444454 | BE018316 | Hs. 11183 | sorting nexin 2 | 3 |
| 448760 | AA313825 | Hs. 21941 | ESTs | 3 |
| 456328 | T41368 | | gb: ph1d1_19/1TV Outward Alu-primed hncDNA lib | 3 |
| 416803 | T79239 | Hs. 168541 | Homo sapiens mRNA full length insert cDNA clo | 3 |
| 418875 | W19971 | Hs. 233459 | ESTs | 3 |
| 432718 | AA563943 | Hs. 244371 | ESTs | 3 |
| 456236 | AF045229 | Hs. 82280 | regulator of G-protein signalling 10 | 3 |
| 402725 | c1p1_6627 | | predicted exon | 3 |
| 408989 | AW361666 | Hs. 49500 | KIAA0746 protein | 3 |
| 430478 | NM_014349 | Hs. 241535 | TNF-inducible protein CG12-1 | 3 |
| 436424 | AA716190 | Hs. 39056 | ESTs | 3 |
| 404345 | AA730407 | Hs. 159156 | protocadherin 11 | 3 |
| 442049 | AA310393 | Hs. 299263 | ESTs | 3 |
| 454119 | BE549773 | Hs. 40510 | uncoupling protein 4 | 3 |
| 417282 | AA195203 | Hs. 479 | RAB5C, member RAS oncogene family | 3 |
| 452436 | BE077546 | Hs. 31447 | ESTs | 3 |
| 410706 | AI732404 | Hs. 68846 | ESTs | 3 |
| 405246 | cNp1_8370 | | predicted exon | 3 |
| 430857 | AW804964 | Hs. 248069 | Human BAC clone CTB-7J15 from 7q31 | 3 |
| 435403 | AA779987 | Hs. 269658 | ESTs | 3 |
| 447982 | H22953 | Hs. 137551 | ESTs | 3 |
| 432680 | T47364 | Hs. 278613 | interferon, alpha-inducible protein 27 | 3 |
| 452774 | AA047374 | Hs. 103388 | ESTs, Weakly similar to I38022 hypothetical p | 3 |
| 417052 | NM_000712 | Hs. 81029 | biliverdin reductase A | 3 |
| 448959 | AI610343 | Hs. 186355 | ESTs | 3 |
| 420299 | AI056871 | Hs. 15276 | ESTs | 3 |
| 409703 | NM_006187 | Hs. 56009 | 2′-5′oligoadenylate synthetase 3 | 3 |
| 454413 | AI653672 | Hs. 40092 | ESTs | 3 |
| 437175 | AW968078 | Hs. 87773 | protein kinase, cAMP-dependent, catalytic, be | 3 |
| 437456 | AL047045 | Hs. 60293 | Homo sapiens clone 122482 unknown mRNA | 3 |
| 453408 | AI804732 | Hs. 295963 | ESTs | 3 |
| 436563 | AJ239450 | Hs. 157874 | ESTs | 3 |
| 448901 | AK001021 | Hs. 22505 | hypothetical protein FLJ10159 | 3 |
| 439649 | T64781 | Hs. 6618 | Homo sapiens cDNA FLJ20782 fis, clone COL0384 | 3 |
| 453887 | BE564037 | Hs. 36237 | CGI-34 protein | 3 |
| 407756 | AA116021 | Hs. 38260 | ubiquitin specific protease 18 | 3 |
| 444222 | AW580955 | Hs. 146236 | ESTs | 3 |
| 416670 | N69267 | Hs. 26073 | ESTs, Moderately similar to HG14_HUMAN NONHIS | 3 |
| 418230 | AI917753 | Hs. 126639 | ESTs | 3 |
| 431049 | AA846576 | Hs. 103267 | hypothetical protein FLJ22548 similar to gene | 3 |
| 417787 | R14948 | Hs. 23883 | ESTs | 3 |
| 421165 | AA284420 | | gb: zs59c08.r1 NCI_CGAP_GCB1 Homo sapiens cDNA | 3 |
| 433297 | AV658581 | Hs. 282633 | ESTs | 3 |
| 445044 | AL137728 | Hs. 12258 | Homo sapiens mRNA; cDNA DKFZp434B0920 (from c | 3 |
| 418945 | BE246762 | Hs. 89499 | arachidonate 5-lipoxygenase | 3 |
| 423706 | U95218 | Hs. 131924 | G protein-coupled receptor 65 | 3 |
| 422630 | AA313606 | Hs. 125509 | hypothetical protein FLJ10648 | 3 |
| 416389 | AA180072 | Hs. 149846 | integrin, beta 5 | 3 |
| 432954 | AI076345 | Hs. 214199 | ESTs, Weakly similar to ALUB_HUMAN !!!! ALU C | 3 |
| 435688 | H72286 | Hs. 128387 | ESTs | 3 |
| 447453 | AW608645 | Hs. 158946 | ESTs | 3 |
| 454278 | AF217525 | Hs. 49002 | Down syndrome cell adhesion molecule | 3 |
| 432485 | N90866 | Hs. 276770 | CDW52 antigen (CAMPATH-1 antigen) | 3 |
| 442584 | AW976853 | Hs. 172843 | ESTs | 3 |
| 426110 | NM_002913 | Hs. 166563 | replication factor C (activator 1) 1 (145 kD) | 3 |
| 450433 | AW444538 | Hs. 231863 | ESTs | 3 |
| 404085 | c6p1_11412 | predicted exon | | |
| 444743 | AA045648 | Hs. 11817 | nudix (nucleoside diphosphate linked moiety X | 3 |
| 416503 | H98502 | Hs. 269853 | ESTs | 3 |
| 421727 | Y13153 | Hs. 107318 | kynurenine 3-monooxygenase (kynurenine 3-hydr | 3 |
| 458082 | AW978811 | Hs. 168213 | ESTs, Weakly similar to ALU1_HUMAN ALU SUBFAM | 3 |
| 437374 | AL359571 | Hs. 12772 | KIAA1565 protein | 3 |
| 428079 | AA421020 | Hs. 208919 | ESTs | 3 |
| 451079 | AI827988 | Hs. 240728 | ESTs | 3 |
| 408212 | AA297567 | Hs. 43728 | hypothetical protein | 3 |
| 417793 | AW405434 | Hs. 82575 | small nuclear ribonucleoprotein polypeptide B | 3 |
| 430697 | AA484207 | Hs. 211867 | ESTs | 3 |
| 453828 | AW970960 | Hs. 293821 | ESTs | 3 |
| 416784 | AA334592 | Hs. 79914 | lumican | 3 |
| 445823 | AI478563 | Hs. 145519 | ESTs | 3 |
| 419586 | AI088485 | Hs. 144759 | ESTs | 3 |
| 440857 | AA907808 | Hs. 135556 | ESTs | 3 |
| 455065 | AW854352 | | gb: RC3-CT0255-200100-024-g10 CT0255 Homo sapi | 3 |
| 450607 | AL050373 | Hs. 25213 | hypothetical protein | 3 |
| 442445 | AA082665 | Hs. 209561 | ESTs, Weakly similar to C05E11.1 gene product | 3 |
| 437868 | F05965 | Hs. 134441 | ESTs | 3 |
| 441664 | AW748420 | Hs. 6236 | Homo sapiens cDNA: FLJ21487 fis, clone COL054 | 3 |
| 409461 | AA382169 | Hs. 54483 | N-myc (and STAT) interactor | 3 |
| 409977 | AW805510 | Hs. 97056 | hypothetical protein FLJ21634 | 3 |
| 446266 | AI417271 | Hs. 163949 | ESTs | 3 |
| 429623 | NM_005308 | Hs. 211569 | G protein-coupled receptor kinase 5 | 3 |
| 447540 | AL135716 | Hs. 263780 | ESTs | 3 |
| 441021 | AW578716 | Hs. 7644 | H1 histone family, member 2 | 3 |
| 448766 | AI473827 | Hs. 31793 | ESTs | 3 |
| 413278 | BE563085 | Hs. 833 | interferon-stimulated protein, 15 kDa | 3 |
| 450293 | N36754 | Hs. 171118 | Homo sapiens mRNA for FLJ00026 protein, parti | 3 |
| 421908 | AW935200 | Hs. 243852 | ESTs, Weakly similar to ALU5_HUMAN ALU SUBFAM | 3 |
| 414915 | NM_002462 | Hs. 76391 | myxovirus (influenza) resistance 1, homolog o | 3 |
| 414489 | AI620677 | Hs. 154191 | ESTs | 3 |
| 433637 | AW024214 | Hs. 135405 | ESTs | 3 |
| 446428 | AW082270 | Hs. 210617 | ESTs, Weakly similar to ALU4_HUMAN ALU SUBFAM | 2 |
| 424528 | AW073971 | Hs. 238954 | ESTs, Weakly similar to KIAA1204 protein [H.s | 2 |
| 419034 | NM_002110 | Hs. 89555 | hemopoietic cell kinase | 2 |
| 419138 | U48508 | Hs. 89631 | ryanodine receptor 1 (skeletal) | 2 |
| 405031 | H25530 | Hs. 50868 | solute carrier family 22 (organic cation tran | 2 |
| 438459 | T49300 | Hs. 35304 | Homo sapiens cDNA FLJ13655 fis, clone PLACE10 | 2 |
| 429752 | H52348 | Hs. 36636 | ESTs | 2 |
| 418827 | BE327311 | Hs. 47166 | EST; HT021 mRNA | 2 |
| 423855 | AA331761 | Hs. 254859 | ESTs | 2 |
| 442989 | BE567710 | | gb: 601340367F1 NIH_MGC_53 Homo sapiens cDNA c | 2 |
| 438493 | AI130740 | Hs. 6241 | phosphoinositide-3-kinase, regulatory subunit | 2 |
| 420338 | AA825595 | Hs. 88269 | ESTs, Highly similar to GPRI_HUMAN PROBABLE G | 2 |
| 432610 | BE246615 | Hs. 278507 | histidyl-tRNA synthetase-like | 2 |
| 431629 | AU077025 | Hs. 265827 | interferon, alpha-inducible protein (clone IF | 2 |
| 458752 | AW292842 | Hs. 255128 | ESTs | 2 |
| 405955 | ph0_1394 | | predicted exon | 2 |
| 430320 | BE245290 | Hs. 239218 | uncharacterized hypothalamus protein HCDASE | 2 |
| 441892 | AB028981 | Hs. 8021 | KIAA1058 protein | 2 |
| 424030 | AB015046 | Hs. 137580 | xylulokinase (H. influenzae) homolog | 2 |
| 407347 | AA829847 | Hs. 167347 | ESTs, Weakly similar to ALU8_HUMAN ALU SUBFAM | 2 |
| 416517 | AA775987 | Hs. 79357 | proteasome (prosome, macropain) 26S subunit, | 2 |
| 415000 | AW025529 | Hs. 239812 | ESTs, Weakly similar to CALM_HUMAN CALMODULIN | 2 |
| 451652 | AA018968 | Hs. 133536 | ESTs | 2 |
| 435497 | AW021655 | Hs. 194441 | ESTs | 2 |
| 448554 | NM_016169 | Hs. 21431 | suppressor of fused | 2 |
| 408405 | AK001332 | Hs. 44672 | hypothetical protein FLJ10470 | 2 |
| 447769 | AW873704 | Hs. 48764 | ESTs | 2 |
| 447514 | AI809314 | Hs. 208501 | ESTs | 2 |
| 430291 | AV660345 | Hs. 238126 | CGI-49 protein | 2 |
| 426108 | AA622037 | Hs. 166468 | programmed cell death 5 | 2 |
| 410240 | AL157424 | Hs. 61289 | synaptojanin 2 | 2 |
| 415579 | AA165232 | Hs. 222069 | ESTs | 2 |
| 418838 | AW385224 | Hs. 35198 | ESTs | 2 |
| 427528 | AU077143 | Hs. 179565 | minichromosome maintenance deficient (S. cere | 2 |
| 452696 | AI826645 | Hs. 211534 | ESTs | 2 |
| 459252 | AF043467 | Hs. 32893 | neurexophilin 2 | 2 |
| 408360 | AI806090 | Hs. 44344 | hypothetical protein FLJ20534 | 2 |
| 414511 | AA148725 | Hs. 12969 | hypothetical protein | 2 |
| 422938 | NM_001809 | Hs. 1594 | centromere protein A (17 kD) | 2 |
| 452670 | AF068227 | Hs. 30213 | ceroid-lipofuscinosis, neuronal 5 | 2 |
| 407332 | AI801565 | Hs. 200113 | Homo sapiens cDNA FLJ11379 fis, clone HEMBA10 | 2 |
| 402107 | c18p1_7398 | predicted exon | | |
| 413048 | M93221 | Hs. 75182 | mannose receptor, C type 1 | 2 |
| 431863 | AA188185 | Hs. 271871 | Spindlin | 2 |
| 416450 | AA180467 | Hs. 142556 | ESTs | 2 |
| 419369 | W28557 | | gb: 48d8 Human retina cDNA randomly primed sub | 2 |
| 447023 | AA356764 | Hs. 17109 | integral membrane protein 2A | 2 |
| 449420 | AI654852 | Hs. 196562 | ESTs, Highly similar to TS24 MOUSE PROTEIN TS | 2 |
| 418721 | NM_002731 | Hs. 87773 | protein kinase, cAMP-dependent, catalytic, be | 2 |
| 441941 | AI953261 | Hs. 169813 | ESTs | 2 |
| 408393 | AW015318 | Hs. 23165 | ESTs | 2 |
| 450746 | D82673 | Hs. 169921 | general transcription factor II, i, pseudogen | 2 |
| 452598 | AI831594 | Hs. 68647 | ESTs, Weakly similar to ALU7_HUMAN ALU SUBFAM | 2 |
| 427719 | AI393122 | Hs. 134726 | ESTs | 2 |
| 452852 | AK001972 | Hs. 30822 | hypothetical protein FLJ11110 | 2 |
| 440266 | AA088809 | Hs. 19525 | hypothetical protein FLJ22794 | 2 |
| 423613 | AF036035 | Hs. 129910 | hyaluronoglucosaminidase 3 | 2 |
| 443601 | AI078554 | Hs. 15682 | ESTs | 2 |
| 432005 | AA524190 | Hs. 120777 | ESTs, Weakly similar to ELL2_HUMAN RNA POLYME | 2 |
| 427794 | AA709186 | Hs. 111973 | ESTs | 2 |
| 417831 | H16423 | Hs. 82685 | CD47 antigen (Rh-related antigen, integrin-as | 2 |
| 435981 | H74319 | Hs. 188620 | ESTs | 2 |
| 408077 | AL133574 | Hs. 42458 | Homo sapiens mRNA; cDNA DKFZp586C1817 (from c | 2 |
| 426312 | AF026939 | Hs. 181874 | interferon-induced protein with tetratricopep | 2 |
| 440561 | AA471379 | Hs. 7277 | peroxisomal biogenesis factor 3 | 2 |
| 407748 | AL079409 | Hs. 38176 | KIAA0606 protein; SCN Circadian Oscillatory P | 2 |
| 407213 | T16206 | Hs. 237164 | ESTs, Highly similar to LDHH_HUMAN L-LACTATE | 2 |
| 449204 | AB000099 | Hs. 23251 | Down syndrome critical region gene 4 | 2 |
| 433364 | AI075407 | Hs. 296083 | ESTs | 2 |
| 419216 | AU076718 | Hs. 164021 | small inducible cytokine subfamily B (Cys-X-C | 2 |
| 425987 | AW015005 | Hs. 165662 | KIAA0675 gene product | 2 |
| 401263 | AB033113 | Hs. 50187 | KIAA1287 protein | 2 |
| 452194 | AI694413 | Hs. 298262 | ESTs, Weakly similar to dJ88J8.1 [H. sapiens] | 2 |
| 415668 | AW957684 | Hs. 77324 | eukaryotic translation termination factor 1 | 2 |
| 401069 | c11p3_633 | | predicted exon | 2 |
| 416475 | T70298 | | gb: yd26g02.s1 Soares fetal liver spleen 1NFLS | 2 |
| 443303 | U67319 | Hs. 9216 | caspase 7, apoptosis-related cysteine proteas | 2 |
| 423095 | S75989 | Hs. 123639 | solute carrier family 6 (neurotransmitter tra | 2 |
| 437575 | AW954355 | Hs. 36529 | ESTs | 2 |
| 458679 | AW975460 | Hs. 143563 | ESTs | 2 |
| 446506 | AI123118 | Hs. 15159 | transmembrane proteolipid | 2 |
| 409132 | AJ224538 | Hs. 50732 | protein kinase, AMP-activated, beta 2 non-cat | 2 |
| 425508 | AA991551 | Hs. 97013 | ESTs | 2 |
| 419644 | AU076951 | Hs. 91797 | retinoblastoma-binding protein 1 | 2 |
| 427639 | AW444530 | Hs. 105362 | ESTs | 2 |
| 427209 | H06509 | Hs. 92423 | KIAA1566 protein | 2 |
| 423235 | AW410698 | | gb: fh07h04.x1 NIH_MGC_17 Homo sapiens cDNA cl | 2 |
| 434961 | AW974956 | | gb: EST387061 MAGE resequences, MAGN Homo sapi | 2 |
| 437594 | AA761431 | Hs. 283318 | ESTs | 2 |
| 407687 | AK002011 | Hs. 37558 | hypothetical protein FLJ11149 | 2 |
| 443119 | AA312264 | Hs. 7980 | ESTs, Moderately similar to ALU4_HUMAN ALU SU | 2 |
| 446591 | H44186 | Hs. 15456 | PDZ domain containing 1 | 2 |
| 442160 | AI337127 | Hs. 156325 | ESTs | 2 |
| 406475 | ph2_23228 | | predicted exon | 2 |
| 448965 | AF092134 | Hs. 22679 | CGI-24 protein | 2 |
| 424243 | AI949359 | Hs. 301837 | ESTs, Highly similar to cis Golgi-localized c | 2 |
| 442048 | AA974603 | | gb: op34f05.s1 Soares_NFL_T_GBC_S1 Homo sapien | 2 |
| 412530 | AA766268 | Hs. 266273 | Homo sapiens cDNA FLJ13346 fis, clone OVARC10 | 2 |
| 447922 | Z92910 | Hs. 20019 | hemochromatosis | 2 |
| 415277 | R44607 | Hs. 22672 | ESTs | 2 |
| 450770 | AA019924 | Hs. 28803 | ESTs | 2 |
| 446946 | AI878932 | Hs. 317 | topoisomerase (DNA) I | 2 |
| 448569 | BE382657 | Hs. 21486 | signal transducer and activator of transcript | 2 |
| 427581 | NM_014788 | Hs. 179703 | KIAA0129 gene product | 2 |
| 407645 | AW062509 | | gb: MR0-CT0069-120899-001-b12 CT0069 Homo sapi | 2 |
| 408179 | AL042465 | Hs. 43445 | poly(A)-specific ribonuclease (deadenylation | 2 |
| 402716 | c1p1_6479 | | predicted exon | 2 |
| 447474 | AW614220 | Hs. 189402 | ESTs | 2 |
| 452705 | H49805 | Hs. 246005 | ESTs | 2 |
| 436643 | AA757626 | Hs. 10941 | ESTs, Weakly similar to IPP1_HUMAN PROTEIN PH | 2 |
| 451625 | R56793 | Hs. 106576 | ESTs | 2 |
| 448233 | AI478114 | Hs. 190615 | ESTs | 2 |
| 427094 | AB025254 | Hs. 283761 | tudor repeat associator with PCTAIRE 2 | 2 |
| 438011 | BE466173 | Hs. 145696 | splicing factor (CC1.3) | 2 |
| 430024 | AI808780 | Hs. 227730 | integrin, alpha 6 | 2 |
| 424840 | D79987 | Hs. 153479 | extra spindle poles, S. cerevisiae, homolog o | 2 |
| 447547 | NM_007229 | Hs. 18842 | protein kinase C and casein kinase substrate | 2 |
| 418259 | AA215404 | Hs. 137289 | ESTs | 2 |
| 449119 | AI631195 | Hs. 232193 | ESTs | 2 |
| 417377 | NM_016603 | Hs. 82035 | GAP-like protein | 2 |
| 415535 | T65331 | | gb: yc74e08.r1 Soares infant brain 1NIB Homo s | 2 |
| 455225 | AW996689 | | gb: QV3-BN0046-150400-151-g09 BN0046 Homo sapi | 2 |
| 404015 | c5p1_9512 | | predicted exon | 2 |
| 419515 | S81944 | Hs. 90791 | gamma-aminobutyric acid (GABA) A receptor, al | 2 |
| 438874 | H02780 | | gb: yj41a11.r1 Soares placenta Nb2HP Homo sapi | 2 |
| 457625 | T10073 | | gb: seq1293 b4HB3MA Cot8-HAP-Ft Homo sapiens c | 2 |
| 444151 | AW972917 | Hs. 128749 | alpha-methylacyl-CoA racemase | 2 |
| 408072 | BE005566 | Hs. 16773 | Homo sapiens clone TCCCIA00427 mRNA sequence | 2 |
| 423568 | NM_005256 | Hs. 129818 | growth arrest-specific 2 | 2 |
| 455514 | AW983871 | | gb: RC1-HN0003-220300-021-h07 HN0003 Homo sapi | 2 |
| 454167 | AW176543 | | gb: MR0-CT0062-200899-002-b04 CT0062 Homo sapi | 2 |
| 414792 | BE314949 | Hs. 235775 | ESTs | 2 |
| 453779 | N35187 | Hs. 43388 | ESTs | 2 |
| 422932 | AI191813 | | gb: qd47f06.x1 Soares_fetal_heart_NbHH19W Homo | 2 |
| 421257 | BE298539 | Hs. 15536 | ESTs, Weakly similar to CNBP_HUMAN CELLULAR N | 2 |
| 408411 | C15118 | Hs. 251967 | Homo sapiens clone 785627 unknown mRNA | 2 |
| 408587 | AW238039 | Hs. 253909 | ESTs | 2 |
| 405545 | cNp3_24204 | predicted exon | | |
| 408683 | R58665 | Hs. 46847 | TRAF and TNF receptor-associated protein | 2 |
| 405392 | cNp3_16759 | predicted exon | | |
| 437613 | R19892 | Hs. 10267 | MIL1 protein | 2 |
| 439645 | BE091801 | Hs. 27167 | ESTs | 2 |
| 457498 | AI732230 | Hs. 191737 | ESTs | 2 |
| 449567 | AI990790 | Hs. 188614 | ESTs | 2 |
| 418791 | AA935633 | Hs. 194628 | ESTs | 2 |
| 437838 | AI307229 | Hs. 184304 | ESTs | 2 |
| 447735 | AA775268 | Hs. 6127 | Homo sapiens cDNA: FLJ23020 fis, clone LNG009 | 2 |
| 416240 | NM_001981 | Hs. 301245 | Homo sapiens clone 23743 mRNA sequence | 2 |
| 424321 | W74048 | Hs. 1765 | lymphocyte-specific protein tyrosine kinase | 2 |
| 452664 | AA398859 | Hs. 18397 | Homo sapiens cDNA: FLJ23221 fis, clone ADSU01 | 2 |
| 433370 | AI084343 | Hs. 122310 | ESTs | 2 |
| 439559 | AW364675 | Hs. 173921 | ESTs | 2 |
| 413129 | AF292100 | Hs. 104613 | RP42 homolog | 2 |
| 445786 | AW629819 | Hs. 144502 | Homo sapiens cDNA: FLJ22055 fis, clone HEP096 | 2 |
| 453469 | AB014533 | Hs. 33010 | KIAA0633 protein | 2 |
| 458020 | AW515443 | Hs. 249495 | heterogeneous nuclear ribonucleoprotein A1 | 2 |
| 412019 | AA485890 | Hs. 69330 | Homo sapiens cDNA FLJ13835 fis, clone THYRO10 | 2 |
| 448873 | NM_003677 | Hs. 22393 | density-regulated protein | 2 |
| 409549 | AB029015 | Hs. 54886 | phospholipase C, epsilon 2 | 2 |
| 450669 | AL138077 | Hs. 16157 | hypothetical protein FLJ12707 | 2 |
| 433017 | Y15067 | Hs. 279914 | zinc finger protein 232 | 2 |
| 435726 | BE535787 | Hs. 113170 | ESTs | 2 |
| 434568 | AA584069 | Hs. 222027 | ESTs | 2 |
| 402524 | c1p1_17748 | predicted exon | | |
| 428388 | AA729827 | Hs. 101265 | Homo sapiens cDNA: FLJ22593 fis, clone HSI032 | 2 |
| 453085 | AW954243 | Hs. 170218 | KIAA0251 protein | 2 |
| 449082 | BE387561 | Hs. 22981 | DKFZP586M1523 protein | 2 |
| 422459 | K02100 | Hs. 117050 | ornithine carbamoyltransferase | 2 |
| 430007 | NM_014892 | Hs. 227602 | KIAA1116 protein | 2 |
| 421494 | AI763322 | Hs. 152104 | ESTs | 2 |
| 422241 | Y00062 | Hs. 170121 | protein tyrosine phosphatase, receptor type, | 2 |
| 401445 | c14p3_4294 | predicted exon | | |
| 414747 | U30872 | Hs. 77204 | centromere protein F (350/400 kD, mitosin) | 2 |
| 416980 | AA381133 | Hs. 80684 | high-mobility group (nonhistone chromosomal) | 2 |
| 419270 | NM_005232 | Hs. 89839 | EphA1 | 2 |
| 447164 | AF026941 | Hs. 17518 | Homo sapiens cig5 mRNA, partial sequence | 2 |
| 452576 | AB023177 | Hs. 29900 | KIAA960 protein | 2 |

Pkey: Unique Eos probeset identifier number
ExAccn: Exemplar Accession number, Genbank accession number
UnigeneID: Unigene number
Unigene Title: Unigene gene title
R1: Diseased Liver: Non-diseased Liver

TABLE 1B

| Pkey | CAT Number | Accession |
|---|---|---|
| 455743 | 1496056_1 | BE073795 BE073756 BE073796 BE073754 BE073752 BE073755 BE073733 BE073753 BE073704 |
| | | BE073695 BE073791 |
| 413541 | 1519670_1 | BE147036 BE146951 BE146976 BE146966 BE146958 BE146955 |
| 410527 | 1030469_1 | AW851066 AW851076 AW851065 AW752861 BF511007 AW851140 AW851166 AW999129 AW850779 |
| | | AW850786 |
| 411466 | 1085200_1 | AW847669 AW847667 AW847668 BE145799 |
| 447513 | 450115_1 | AW955776 AW264910 AI401003 AI382588 D20260 N74904 H57056 R26462 AV735490 |
| 456383 | 250410_1 | AA236756 AA287178 AI148037 |
| 450560 | 50855_1 | NM_024331 BC003071 BM012414 BE315221 BG750119 BE272198 AL449476 BE886722 AI360302 |
| | | BG002949 BM454474 AL449598 BM012506 |
| | | BE383204 AA010225 AL449685 AL449684 BF742320 T06328 |
| 419375 | 2390505_1 | W27916 W26506 |
| 410430 | 5482_6 | BG120564 BG705653 BF846503 BF995692 BE311644 |
| 454750 | 1070828_1 | AW866285 AW866541 AW819153 AW819000 AW819014 |
| 411857 | 1112533_1 | AW867707 AW879403 |
| 455960 | 1554632_1 | BE165256 BE165247 BE165239 BE165233 BE165264 BE165262 BE165261 BE165252 BE165251 |
| | | BE165245 BE165203 BE165201 BE165242 |
| | | BE165206 BE165175 BE165232 BE165184 BE165197 BE165194 BE165193 BE165246 BE165240 BE165186 |
| | | BE165237 BE165180 |
| 442431 | MH1944_19 | AW886349 AI818145 BE463452 BF002624 AI360447 AI634842 AI362712 BE349856 W74084 AW014214 |
| | | W72374 AA995742 R80905 R80906 |
| 433854 | 899720_1 | BG675161 H59558 AI699484 AA610649 AI937812 |
| 431300 | 1529181_1 | BE159863 AA502346 AU186097 R86267 H71358 |
| 410833 | 1061214_1 | AW806900 BF373960 BF373956 AW866317 AW866524 AW866625 BF373959 AW866592 |
| 455708 | 1493321_1 | BE069290 BE069352 BE069326 |
| 425195 | 12922_3 | BG197420 BG219369 BG182827 AA352026 |
| 457707 | 114453_1 | AA640546 AW974642 AA649509 AA649527 |
| 413605 | 1523960_1 | BE152811 BE152651 BE152644 BE152659 BE152810 BE152714 BE152707 BE152643 BE152660 BE152669 |
| | | BE152711 BE152808 BE152782 |
| | | BE152678 BE152682 BE152813 BE152778 BF350474 BE152776 BE152781 BE152774 BF350475 BE152712 |
| | | BE152706 BE152668 BE152814 |
| | | BE152671 BE152652 BE152760 BE152767 BE152775 BE152815 BE152715 BE152681 BE152771 BE152661 |
| | | BE152780 BE152763 BE152666 |
| | | BE152708 BE152665 BE152664 BE152677 BE152662 BE152768 BE152709 BE152679 BE152667 BE152673 |
| | | BE152676 BE152656 BE152769 |
| | | BE152816 BE152809 BE152672 BE152653 BE152716 BE152762 |
| 433586 | 32908_1 | BC011194 AW517087 AA601054 T85512 |
| 454314 | 773174_1 | AW364844 AW364847 AW937534 AW937593 AW937659 |
| 456328 | 2306193_1 | T41294 T41304 T41368 T41369 |
| 421165 | 50467_2 | BG620396 AA428945 H89283 AA831889 AI039537 BG573209 AA284420 AI267186 H97302 |
| 455065 | 1094993_1 | AW854352 AW854461 AW854311 AW854340 |
| 442989 | 1768039_1 | BE567710 R02368 |
| 419369 | 856237_1 | W28557 BG619281 W26273 |
| 416475 | 1972665_1 | R02750 H58072 T70298 BF367306 R02749 T80873 |
| 423235 | 7447_1 | BC016162 AK054907 BC008564 BC011232 AL533635 BF508705 AA521407 AA521325 AI400703 |
| | | AI439041 BF726761 AI440391 AW451413 |
| | | AI127908 BE463710 AI076067 AI380502 AI249172 AI475513 AI932260 AA598632 AW503511 BF111247 |
| | | AA516001 AI435214 AW295486 |
| | | AI251854 BI963232 AI242565 AI247008 AI621262 AI766708 AJ401189 NM_032595 AI523759 AW028349 |
| | | AW302139 AI808223 AI475761 |
| | | BE895415 BI912507 AA323578 BF951255 BF947949 AA323535 BF947948 BF448737 BF515503 BF109903 |
| | | AI969706 AI356745 BF476688 |
| | | AI475690 AW082861 AI056581 AI521266 AA889737 BM461771 BM129164 BI909136 BF874611 BE909797 |
| | | BI755397 BI052380 BF973595 |
| | | BI052420 BI012504 BF806956 BI052419 AA444012 BF848781 BF091592 AA884981 |
| 434961 | 121331_1 | AA781075 AA654944 AW974956 |
| 442048 | 750422_1 | AW340495 AI984319 AA974603 |
| 407645 | 579939_1 | AW062509 AW845614 BE140931 AW845635 |
| 415535 | 1875630_1 | T65331 F11774 F11773 |
| 455225 | 1113920_1 | AW868687 AW996453 AW996689 AW996380 BE085650 BE085595 |
| 438874 | 52147_1 | AF075017 R66779 R22463 H02780 |
| 457625 | 433710_1 | H14872 T10073 AV723827 AA604786 |
| 455514 | 1243022_1 | AW983860 BE090302 AW983845 AW983853 AW983871 AW983867 AW983852 |
| 454167 | 1048866_1 | AW176543 AW806978 BE141056 AW806985 AW178964 AW845681 |
| 422932 | 9154_11 | H83343 AW954934 AA417867 AA319212 |

Pkey: Unique Eos probeset identifier number
CAT number: Gene cluster number
Accession: Genbank accession numbers

TABLE 1C

| | | | |
|---|---|---|---|
| 402811 | 6523646 | Plus | 101679-101844 |
| 403747 | 7658395 | Minus | 20493-20621 |
| 406367 | 9256126 | Minus | 58313-58489 |
| 404287 | 2326514 | Plus | 53134-53281 |
| 402725 | 8979991 | Plus | 107231-107383 |
| 405246 | 7249293 | Minus | 82725-82884 |
| 405955 | 6758797 | Plus | 39940-40092 |
| 401069 | 3927852 | Minus | 45682-45831 |
| 406475 | 9797684 | Plus | 125417-125563, 128052-128180 |
| 402716 | 8969253 | Minus | 84065-84242 |
| 404015 | 8655948 | Minus | 587821-588222 |

Pkey: Unique number corresponding to an Eos probeset
Ref: Sequence source. The 7 digit numbers in this column are Genbank Identifier (GI) numbers. “Dunham, et al.” refers to the publication entitled “The DNA sequence of human chromosome 22” Dunham, et al. (1999) Nature 402: 489-495.
Strand: Indicates DNA strand from which exons were predicted.
Nt_position: Indicates nucleotide positions of predicted exons.

TABLE 2A
ABOUT 535 GENES DOWNREGULATED IN HEPATITIS C [see 60/308, 188]

| Pkey | ExAccn | UnigeneID | Title | Ratio |
|---|---|---|---|---|
| 450912 | AW939251 | Hs. 25647 | v-fos FBJ murine osteosarcoma viral onco | 2 |
| 447078 | AW885727 | Hs. 301570 | ESTs | 2 |
| 442941 | AU076728 | Hs. 8867 | cysteine-rich, angiogenic inducer, 61 | 2 |
| 419564 | U08989 | Hs. 91139 | solute carrier family 1 (neuronal/epithe | 2 |
| 447771 | BE505004 | Hs. 280838 | ESTs | 2 |
| 444286 | AI625304 | Hs. 190312 | ESTs | 2 |
| 436711 | AW452601 | Hs. 189907 | ESTs | 2 |
| 434078 | AW880709 | Hs. 283683 | EST | 2 |
| 442570 | AI001834 | Hs. 130264 | ESTs | 2 |
| 453270 | AI971439 | Hs. 233461 | ESTs | 2 |
| 410140 | AL134435 | Hs. 27872 | ESTs | 2 |
| 404839 | cAp3_4046 | | | 2 |
| 418138 | AA213626 | Hs. 136204 | EST | 2 |
| 444541 | AI161257 | Hs. 167252 | ESTs | 2 |
| 408098 | R61857 | Hs. 120981 | ESTs | 2 |
| 400959 | c11p1_3967 | | | 2 |
| 403397 | c3p1_11672 | | | 2 |
| 440948 | AW188311 | Hs. 128619 | ESTs | 2 |
| 444648 | AI221297 | Hs. 147778 | ESTs, Weakly similar to KIAA0454 protein | 2 |
| 404723 | c9p1_8723 | | | 2 |
| 451400 | BE160479 | | gb: QV1-HT0413-210200-081-g05 HT0413 Homo | 2 |
| 432228 | AA335178 | Hs. 274124 | Human DNA sequence from clone 1018D12 on | 2 |
| 415328 | Z44310 | | gb: HSC1XF011 normalized infant brain cDN | 2 |
| 445967 | D59597 | Hs. 118821 | CGI-62 protein | 2 |
| 444776 | AI191980 | Hs. 145430 | ESTs | 2 |
| 447875 | R22029 | Hs. 13905 | ESTs | 2 |
| 408834 | AW276241 | | gb: xr08f06.x1 NCI_CGAP_Lu28 Homo sapiens | 2 |
| 458099 | AW263124 | Hs. 34782 | ESTs | 2 |
| 440634 | AA921767 | Hs. 132447 | ESTs | 2 |
| 442786 | H50733 | Hs. 256261 | ESTs | 2 |
| 415477 | NM_002228 | Hs. 78465 | v-jun avian sarcoma virus 17 oncogene ho | 2 |
| 407516 | X64974 | | gb: H. sapiens mRNA HTPCRH02 for olfactory | 2 |
| 455806 | BE141094 | | gb: MR0-HT0075-121199-004-e05 HT0075 Homo | 2 |
| 421370 | AA287904 | Hs. 269669 | ESTs | 2 |
| 430737 | AW364181 | Hs. 208763 | ESTs | 2 |
| 404398 | c7p3_782 | | | 2 |
| 418998 | F13215 | Hs. 287849 | ESTs | 2 |
| 426566 | AF131836 | Hs. 170453 | tropomodulin | 2 |
| 445225 | AI216555 | Hs. 202398 | ESTs | 2 |
| 455181 | AW863568 | | gb: MR3-SN0010-240300-102-c10 SN0010 Homo | 2 |
| 457752 | AI821270 | Hs. 116930 | ESTs | 2 |
| 405475 | cNp3_19914 | | | 2 |
| 423167 | AA770464 | | gb: ah89g09.s1 Soares_NFL_T_GBC_S1 Homo s | 2 |
| 414559 | AV656184 | Hs. 76452 | C-reactive protein, pentraxin-related | 2 |
| 459263 | L25475 | | gb: HUM21ES116 ClonTech HL 1065a Home sap | 2 |
| 455175 | AW993247 | | gb: RC2-BN0033-180200-014-h09 BN0033 Homo | 2 |
| 454448 | AW750209 | | gb: RC5-BT0562-260100-011-H03 BT0562 Homo | 2 |
| 417566 | T81449 | Hs. 191199 | ESTs | 2 |
| 404498 | c8p1_4579 | | | 2 |
| 418501 | BE079398 | Hs. 5921 | Homo sapiens cDNA: FLJ21592 fis, clone C | 2 |
| 409113 | AA074897 | | gb: zm85a05.r1 Stratagene ovarian cancer | 2 |
| 401074 | c11p3_815 | | | 2 |
| 456304 | AI820973 | Hs. 188706 | ESTs | 2 |
| 459689 | AA584858 | | ESTs | 2 |
| 423669 | AA329417 | Hs. 272321 | Homo sapiens cDNA FLJ12571 fis, clone NT | 2 |
| 441884 | AW172630 | Hs. 144884 | ESTs | 2 |
| 441837 | AA361743 | Hs. 179881 | core-binding factor, beta subunit | 2 |
| 400371 | U80740 | Hs. 278692 | trinucleotide repeat containing 8 | 2 |
| 439034 | AF075083 | | gb: Homo sapiens full length insert cDNA | 2 |
| 439075 | AF085933 | Hs. 292620 | ESTs | 2 |
| 415350 | R13218 | | gb: yf74c05.r1 Soares infant brain 1NIB H | 2 |
| 427668 | AA298760 | Hs. 180191 | Homo sapiens mRNA; cDNA DKFZp434L0217 (f | 2 |
| 457103 | AI421187 | Hs. 189192 | ESTs | 2 |
| 433970 | AA721401 | Hs. 301908 | ESTs | 2 |
| 442314 | AI311854 | Hs. 129220 | ESTs | 2 |
| 435332 | AA678019 | Hs. 187994 | ESTs | 2 |
| 451740 | R63962 | Hs. 269210 | ESTs | 2 |
| 458198 | AI286100 | Hs. 192739 | ESTs | 2 |
| 428959 | AF100779 | Hs. 194680 | WNT1 inducible signaling pathway protein | 2 |
| 445211 | BE045601 | Hs. 118248 | ESTs, Weakly similar to YC18_HUMAN HYPOT | 2 |
| 459695 | AA381579 | | ESTs | 2 |
| 411355 | AW838479 | Hs. 22692 | ESTs | 2 |
| 401887 | c17p1_704 | | | 2 |
| 406285 | AW068311 | Hs. 82582 | integrin, beta-like 1 (with EGF-like rep | 2 |
| 439201 | AW503578 | Hs. 209406 | ESTs, Weakly similar to Z140_HUMAN ZINC | 2 |
| 407760 | T79084 | Hs. 184407 | ESTs | 2 |
| 451886 | T63790 | Hs. 293720 | Homo sapiens cDNA: FLJ22804 fis, clone K | 2 |
| 423657 | AL045128 | Hs. 1691 | glucan (1,4-alpha-), branching enzyme 1 | 2 |
| 426529 | AF090100 | Hs. 170241 | Homo sapiens clone IMAGE 23915 | 2 |
| 440728 | AW086077 | Hs. 153272 | Homo sapiens cDNA: FLJ22715 fis, clone H | 2 |
| 445061 | AI253094 | Hs. 145227 | ESTs | 2 |
| 421013 | M62397 | Hs. 1345 | mutated in colorectal cancers | 2 |
| 430873 | AW269813 | Hs. 154395 | ESTs | 2 |
| 407850 | AW086230 | Hs. 244912 | ESTs | 2 |
| 426077 | AA448328 | Hs. 115527 | ESTs | 2 |
| 419927 | R53365 | Hs. 20001 | ESTs | 2 |
| 433945 | AI024718 | Hs. 112873 | ESTs | 2 |
| 434554 | R13594 | Hs. 301529 | ESTs | 2 |
| 418625 | AW948578 | Hs. 136211 | ESTs | 2 |
| 453523 | NM_012118 | Hs. 258586 | CCR4-like (carbon catabolite repression | 2 |
| 455195 | AW864370 | | gb: PM4-SN0016-100500-004-h09 SN0016 Homo | 2 |
| 406547 | ph2_5308 | | | 2 |
| 416510 | H60055 | Hs. 169833 | single-stranded-DNA-binding protein | 2 |
| 415737 | AA167626 | Hs. 118743 | ESTs | 2 |
| 431895 | H60210 | Hs. 272003 | hemoglobin, zeta | 2 |
| 402468 | c1p1_13012 | | | 2 |
| 454042 | H22570 | Hs. 172572 | hypothetical protein FLJ20093 | 2 |
| 401943 | NM_012434 | Hs. 117865 | solute carrier family 17 (anion/sugar tr | 2 |
| 412065 | R82597 | Hs. 176648 | ESTs | 2 |
| 444601 | AV650521 | Hs. 282449 | ESTs | 2 |
| 438247 | AI018016 | Hs. 131222 | ESTs | 2 |
| 441224 | AU076964 | Hs. 7753 | calumenin | 2 |
| 449463 | AI657038 | Hs. 196109 | ESTs | 2 |
| 451029 | AA852097 | Hs. 25829 | ras-related protein | 2 |
| 419728 | L36861 | Hs. 92858 | guanylate cyctase activator 1A (retina) | 2 |
| 436763 | AI168278 | Hs. 128713 | ESTs | 2 |
| 411861 | AW867875 | | gb: MR0-SN0040-050500-003-f11 SN0040 Homo | 2 |
| 449278 | AI637876 | Hs. 224372 | ESTs | 2 |
| 456576 | AA287443 | | gb: zs52c10.r1 NCI_CGAP_GCB1 Homo sapiens | 2 |
| 432240 | AI694767 | Hs. 129179 | ESTs | 2 |
| 447788 | AI424822 | Hs. 161430 | ESTs | 2 |
| 405278 | cNp3_1070 | | | 2 |
| 408332 | H91230 | Hs. 234794 | Homo sapiens mRNA; cDNA DKFZp564B083 (fr | 2 |
| 454442 | AW816134 | | gb: MR3-ST0220-290100-016-e04 ST0220 Homo | 2 |
| 454520 | AW803371 | | gb: IL2-UM0079-090300-049-B06 UM0079 Homo | 2 |
| 437103 | AW139408 | Hs. 152940 | ESTs | 2 |
| 458254 | BE091969 | Hs. 127742 | ESTs | 2 |
| 415075 | L27479 | Hs. 77889 | Friedreich ataxia region gene X123 | 2 |
| 450577 | AW612816 | Hs. 202057 | ESTs | 2 |
| 414564 | AA164803 | Hs. 71994 | ESTs | 2 |
| 459349 | AW749381 | | gb: QV3-BT0381-170100-060-c02 BT0381 Homo | 2 |
| 450581 | AF081513 | Hs. 25195 | endometrial bleeding associated factor | 2 |
| 445239 | AI217375 | Hs. 170023 | ESTs, Weakly similar to collagen alpha 3 | 2 |
| 421227 | R78581 | Hs. 266308 | ESTs, Weakly similar to AF216312 1 type | 2 |
| 432675 | AI791855 | Hs. 105884 | ESTs | 2 |
| 451831 | NM_001674 | Hs. 460 | activating transcription factor 3 | 2 |
| 455104 | BE064863 | | gb: RC1-BT0313-110300-015-f06 BT0313 Homo | 2 |
| 441445 | AI221959 | Hs. 187937 | ESTs | 2 |
| 405456 | cNp3_18813 | | | 2 |
| 452747 | BE153855 | Hs. 61460 | Ig superfamily receptor LNIR precursor | 2 |
| 449438 | AA927317 | Hs. 176719 | ESTs | 2 |
| 437662 | AA765387 | Hs. 145095 | ESTs | 2 |
| 443258 | AF169301 | Hs. 9098 | sulfate transporter 1 | 2 |
| 448670 | AW296257 | Hs. 230507 | ESTs | 2 |
| 425426 | AB021641 | Hs. 157203 | Homo sapiens GIOT-1 mRNA for gonadotropi | 2 |
| 446438 | AI299876 | Hs. 150061 | ESTs | 2 |
| 457005 | AJ007421 | Hs. 300698 | ESTs, Highly similar to spalt-like zinc | 2 |
| 407473 | L10404 | | gb: Homo sapiens DNA binding protein for | 2 |
| 404319 | c7p1_2230 | | | 2 |
| 429836 | AW117452 | Hs. 99489 | ESTs | 2 |
| 416461 | AA180526 | Hs. 216797 | ESTs | 2 |
| 435185 | AA669490 | Hs. 289109 | dimethylarginine dimethylaminohydrolase | 2 |
| 403600 | c3p1_6888 | | | 2 |
| 401775 | c17p1_11738 | | | 2 |
| 424200 | AA337221 | | gb: EST41944 Endometrial tumor Homo sapie | 2 |
| 405532 | cNp3_23494 | | | 2 |
| 424404 | AA340151 | Hs. 104650 | hypothetical protein FLJ10292 | 2 |
| 427168 | AA398821 | Hs. 97548 | ESTs | 2 |
| 430071 | AA355986 | Hs. 232068 | transcription factor 8 (represses interl | 2 |
| 423290 | AA324130 | | gb: EST27023 Cerebellum II Homo sapiens c | 2 |
| 429295 | AA682377 | Hs. 99216 | ESTs, Moderately similar to ALU8_HUMAN A | 2 |
| 445571 | AI378000 | Hs. 158489 | ESTs, Weakly similar to b3418.1 [H.sapie | 2 |
| 432459 | AW291917 | Hs. 174387 | ESTs | 2 |
| 424584 | H10692 | Hs. 13310 | ESTs | 2 |
| 401411 | c14p3_2875 | | | 2 |
| 429932 | AI095005 | Hs. 135174 | ESTs | 2 |
| 449305 | AI638293 | | gb: tt09b07.x1 NCI_CGAP_GC6 Homo sapiens | 2 |
| 413257 | BE075035 | | gb: PM3-BT0584-260300-002-g05 BT0584 Homo | 2 |
| 436062 | AK000027 | Hs. 98633 | ESTs | 2 |
| 430692 | X80240 | | gb: H. sapiens endogenous retrovirus HERV- | 2 |
| 403212 | c2p1_2196 | | | 2 |
| 424686 | AA345504 | | gb: EST51529 Gall bladder II Homo sapiens | 2 |
| 413272 | AA127923 | Hs. 293256 | ESTs | 2 |
| 411658 | AW855598 | | gb: CM1-CT0278-031199-032-e08 CT0278 Homo | 2 |
| 459721 | AI299050 | | gb: qn14d12.x1 NCI_CGAP_Lu5 Homo sapiens | 2 |
| 404834 | cAp3_3862 | | | 2 |
| 442484 | AF075360 | | gb: AF075360 Human fetal liver cDNA libra | 2 |
| 411689 | AW857121 | | gb: RC1-CT0302-040400-017-a12 CT0302 Homo | 2 |
| 407707 | AW294785 | Hs. 143895 | Homo sapiens cDNA: FLJ21140 fis, clone C | 2 |
| 433430 | AI863735 | Hs. 186755 | ESTs | 2 |
| 401866 | c17p1_5127 | | | 2 |
| 400352 | AF227133 | Hs. 272389 | Homo sapiens candidate taste receptor T2 | 2 |
| 444862 | AI209158 | Hs. 143929 | ESTs | 2 |
| 401558 | c15p1_539 | | | 2 |
| 405698 | cNp3_9135 | | | 2 |
| 427731 | AA411750 | Hs. 20943 | ESTs | 2 |
| 411486 | N85785 | Hs. 181165 | eukaryotic translation elongation factor | 2 |
| 438557 | AW364104 | Hs. 143509 | Homo sapiens cDNA: FLJ21924 fis, clone H | 2 |
| 411902 | AW875344 | | gb: RC1-PT0009-220300-013-f06 PT0009 Homo | 2 |
| 444573 | AW043590 | Hs. 225023 | ESTs | 2 |
| 420355 | AW968263 | Hs. 123126 | ESTs | 2 |
| 418225 | AA747676 | | gb: nx85g05.s1 NCI_CGAP_GCB1 Homo sapiens | 2 |
| 404974 | cNp1_15731 | | | 3 |
| 410993 | BE138999 | Hs. 278868 | ESTs | 3 |
| 410900 | AW810169 | | gb: MR4-ST0124-040500-007-h07 ST0124 Homo | 3 |
| 445626 | AI400253 | Hs. 156240 | ESTs | 3 |
| 449986 | AW864502 | | gb: PM4-SN0016-120400-004-b12 SN0016 Homo | 3 |
| 413088 | BE064962 | | gb: RC1-BT0313-130400-016-c02 BT0313 Homo | 3 |
| 441747 | BE467749 | Hs. 144029 | ESTs, Highly similar to SOX1_HUMAN SOX-1 | 3 |
| 448156 | AI472886 | | gb: tj75d01.x1 Soares_NSF_F8_9W_OT_PA_P_S | 3 |
| 418298 | AA256014 | Hs. 86682 | Homo sapiens cDNA: FLJ21578 fis, clone C | 3 |
| 404196 | c6p3_1867 | | | 3 |
| 452528 | AA742457 | Hs. 291479 | ESTs | 3 |
| 451540 | AI801860 | Hs. 208837 | ESTs | 3 |
| 436893 | AA736815 | Hs. 149225 | ESTs | 3 |
| 401758 | c17p1_10881 | | | 3 |
| 435633 | AI248152 | Hs. 270047 | ESTs | 3 |
| 409955 | U60665 | Hs. 57692 | testis specific basic protein | 3 |
| 440600 | AI807691 | Hs. 126351 | ESTs | 3 |
| 401946 | c17p3_245 | | | 3 |
| 429668 | AA626142 | Hs. 179991 | ESTs, Weakly similar to KPCE_HUMAN PROTE | 3 |
| 424562 | AI420859 | Hs. 150557 | basic transcription element binding prot | 3 |
| 402627 | c1p1_26870 | | | 3 |
| 400400 | AF144054 | Hs. 283886 | Homo sapiens apoptosis related protein A | 3 |
| 413813 | M96956 | Hs. 75561 | teratocarcinoma-derived growth factor 1 | 3 |
| 433851 | AA610436 | Hs. 196461 | ESTs | 3 |
| 432217 | AI864415 | Hs. 162157 | ESTs, Moderately similar to B34087 hypot | 3 |
| 454573 | BE146471 | | gb: QV0-HT0216-011199-043-c09 HT0216 Homo | 3 |
| 432304 | AA932186 | Hs. 164214 | ESTs | 3 |
| 458072 | AI890347 | Hs. 271923 | EST | 3 |
| 443633 | AL031290 | Hs. 9654 | similar to pregnancy-associated plasma p | 3 |
| 454697 | AW813728 | Hs. 15036 | ESTs, Highly similar to AF161358 1 HSPC0 | 3 |
| 415877 | R45135 | Hs. 21026 | ESTs | 3 |
| 458943 | AW249181 | Hs. 19954 | ESTs, Weakly similar to cDNA EST yk386e1 | 3 |
| 431775 | AW205945 | Hs. 27008 | phosphatidylinositol glycan, class L | 3 |
| 433391 | T77201 | | gb: yc95c09.r1 Soares infant brain 1NIB H | 3 |
| 411929 | AA098880 | Hs. 69297 | ESTs | 3 |
| 443728 | AI083876 | Hs. 148383 | ESTs | 3 |
| 449637 | AA001964 | | gb: ze49e02.r1 Soares retina N2b4HR Homo | 3 |
| 436857 | AA732647 | | gb: nz89d01.s1 NCI_CGAP_GCB1 Homo sapiens | 3 |
| 406562 | ph2_6297 | | | 3 |
| 411484 | AW848117 | | gb: IL3-CT0214-301299-048-D04 CT0214 Homo | 3 |
| 456549 | AA283740 | Hs. 89211 | ESTs | 3 |
| 443751 | AI285839 | Hs. 153324 | ESTs | 3 |
| 428515 | AF030339 | Hs. 286229 | plexin C1 | 3 |
| 415727 | BE501389 | Hs. 20848 | ESTs, Weakly similar to U5 snRNP-specifi | 3 |
| 453493 | AL039478 | | gb: DKFZp434P0510_s1 434 (synonym: htes3) | 3 |
| 429505 | AW820035 | Hs. 204290 | Homo sapiens mRNA; cDNA DKFZp586N2119 (f | 3 |
| 432741 | AI732358 | Hs. 185118 | ESTs | 3 |
| 408766 | AA057270 | | gb: zk70c03.r1 Soares_pregnant_uterus_NbH | 3 |
| 455036 | AW851630 | | gb: MR2-CT0222-211099-002-h06 CT0222 Homo | 3 |
| 421036 | AA810560 | | gb: oa71h06.s1 NCI_CGAP_GCB1 Homo sapiens | 3 |
| 416584 | N63864 | Hs. 205554 | ESTs | 3 |
| 420125 | AA255739 | Hs. 283332 | ESTs | 3 |
| 434137 | AA907734 | Hs. 124895 | ESTs | 3 |
| 402747 | c1p1_7488 | | | 3 |
| 457508 | AA542909 | Hs. 162214 | ESTs | 3 |
| 444927 | AW016637 | Hs. 199425 | ESTs | 3 |
| 408443 | N33937 | Hs. 10336 | ESTs | 3 |
| 408033 | AW138045 | Hs. 242256 | ESTs | 3 |
| 425560 | AA359368 | Hs. 165998 | DKFZP564M2423 protein | 3 |
| 444531 | BE158822 | Hs. 282469 | ESTs | 3 |
| 455404 | BE175503 | | gb: RC5-HT0580-050400-021-B01 HT0580 Homo | 3 |
| 458786 | AI457098 | Hs. 280848 | ESTs | 3 |
| 447034 | N49580 | Hs. 46630 | ESTs | 3 |
| 434286 | AF123758 | Hs. 127675 | ceroid-lipofuscinosis, neuronal 8 (epile | 3 |
| 400488 | c10p1_1764 | | | 3 |
| 453502 | AL039786 | | gb: DKFZp434A0912_r1 434 (synonym: htes3) | 3 |
| 406225 | ph0_8239 | | | 3 |
| 424554 | AA747563 | Hs. 131799 | ESTs, Weakly similar to ALU8_HUMAN ALU S | 3 |
| 409854 | AW501833 | | gb: UI-HF-BR0p-ajo-d-01-0-UI.r1 NIH_MGC_5 | 3 |
| 453006 | AI362575 | Hs. 167133 | ESTs | 3 |
| 406531 | ph2_4560 | | | 3 |
| 451333 | AK000914 | Hs. 26244 | hypothetical protein FLJ10052 | 3 |
| 419882 | AA687313 | Hs. 190043 | ESTs | 3 |
| 432073 | AW661883 | Hs. 259353 | ESTs | 3 |
| 426420 | BE383808 | Hs. 169829 | KIAA1180 protein | 3 |
| 437089 | AA844539 | Hs. 240855 | ESTs | 3 |
| 422638 | AI474074 | Hs. 172070 | ESTs, Weakly similar to cAMP-specific cy | 3 |
| 454754 | AW819191 | | gb: CM1-ST0283-071299-061-d08 ST0283 Homo | 3 |
| 429768 | AA805719 | Hs. 192154 | ESTs | 3 |
| 445224 | BE254241 | Hs. 288885 | Homo sapiens cDNA FLJ14246 fis, clone OV | 3 |
| 450684 | AA872605 | Hs. 25333 | interleukin 1 receptor, type II | 3 |
| 435211 | AI248618 | Hs. 193586 | ESTs | 3 |
| 419392 | W28573 | | gb: 51f10 Human retina cDNA randomly prim | 3 |
| 427624 | AA406245 | Hs. 24895 | ESTs | 3 |
| 454803 | AW860148 | | gb: RC0-CT0379-290100-032-b10 CT0379 Homo | 3 |
| 427419 | NM_000200 | Hs. 177888 | histatin 3 | 3 |
| 439884 | H42671 | | gb: yp13h12.r1 Soares breast 3NbHBst Homo | 3 |
| 407438 | AF227133 | | gb: Homo sapiens candidate taste receptor | 3 |
| 427700 | AA262294 | Hs. 180383 | dual specificity phosphatase 6 | 3 |
| 408602 | AA055833 | Hs. 58152 | ESTs, Weakly similar to anagen-specific | 3 |
| 405548 | cNp3_24436 | | | 3 |
| 453916 | AW974874 | Hs. 75212 | omithine decarboxylase 1 | 3 |
| 425611 | AF012270 | Hs. 158338 | retinal pigment epithelium-derived rhodo | 3 |
| 459238 | AF053551 | Hs. 31584 | metaxin 2 | 3 |
| 454816 | AW833258 | | gb: RC2-TT0007-131099-011-b10_1 TT0007 Ho | 3 |
| 443184 | AI638728 | Hs. 131973 | ESTs | 3 |
| 407986 | U32659 | Hs. 41724 | interleukin 17 (cytotoxic T-lymphocyte-a | 3 |
| 405510 | cNp3_22634 | | | 3 |
| 435118 | AA665576 | Hs. 116581 | ESTs | 3 |
| 414041 | AW974100 | Hs. 293265 | ESTs | 3 |
| 450508 | R37408 | Hs. 101654 | ESTs | 3 |
| 417699 | T91491 | Hs. 119670 | ESTs | 3 |
| 438160 | AA779332 | Hs. 122671 | ESTs | 3 |
| 423792 | AW135866 | Hs. 245854 | ESTs | 3 |
| 437208 | AA236599 | | gb: zs42c10.r1 Soares_NhHMPu_S1 Homo sapi | 3 |
| 412234 | AW902641 | | gb: QV3-NN1024-100500-181-d08 NN1024 Homo | 3 |
| 409937 | AI804584 | Hs. 57672 | leucine rich repeat (in FLII) interactin | 3 |
| 410158 | AA082030 | | gb: zn26h08.r1 Stratagene neuroepithelium | 3 |
| 408448 | BE467627 | Hs. 285574 | ESTs | 3 |
| 435308 | N28276 | Hs. 117087 | ESTs | 3 |
| 459472 | AA568933 | | gb: nm23c07.s1 NCI_CGAP_Co10 Homo sapiens | 3 |
| 430826 | U10061 | Hs. 248019 | POU domain, class 4, transcription facto | 3 |
| 403003 | c21p3_2293 | | | 3 |
| 404499 | c8p1_4589 | | | 3 |
| 407530 | X68790 | | gb: H. sapiens bactericidal BPl′gene for a | 3 |
| 456280 | D63477 | Hs. 84087 | KIAA0143 protein | 3 |
| 446418 | AI301117 | Hs. 150182 | ESTs | 3 |
| 420535 | AA280095 | Hs. 88689 | ESTs | 3 |
| 410815 | AW805974 | | gb: QV1-UM0106-130400-152-b10 UM0106 Homo | 3 |
| 447925 | AW292271 | Hs. 250718 | ESTs | 3 |
| 441769 | R62241 | Hs. 172780 | ESTs | 3 |
| 439889 | AA848093 | Hs. 192993 | ESTs | 3 |
| 431525 | AA506656 | Hs. 6185 | KIAA1557 protein | 3 |
| 414294 | BE270795 | Hs. 268864 | ESTs | 3 |
| 451300 | AA017066 | Hs. 237686 | EST | 3 |
| 410204 | AJ243425 | Hs. 738 | early growth response 1 | 3 |
| 413939 | AL047051 | Hs. 199961 | ESTs | 3 |
| 425925 | AF176813 | Hs. 163045 | soluble adenylyl cyclase | 3 |
| 418088 | R49517 | Hs. 268703 | ESTs | 3 |
| 436681 | AI288242 | | gb: ql80b03.x1 Soares_NhHMPu_S1 Homo sapi | 3 |
| 453565 | BE298808 | Hs. 33363 | DKFZP434N093 protein | 3 |
| 420568 | F09247 | Hs. 167399 | protocadherin alpha 5 | 3 |
| 444564 | AI167877 | Hs. 143716 | ESTs | 3 |
| 436317 | AL096777 | | gb: Novel human gene mapping to chomosome | 3 |
| 441299 | AA927914 | Hs. 223718 | ESTs | 3 |
| 443338 | R99575 | | gb: yq72c01.s1 Soares fetal liver spleen | 3 |
| 410286 | AI739159 | Hs. 61898 | DKFZP586N2124 protein | 3 |
| 416659 | W22048 | | gb: 61A12 Human retina cDNA Tsp509I-cleav | 3 |
| 435070 | AI821270 | Hs. 116930 | ESTs | 3 |
| 449877 | BE408252 | Hs. 301008 | ESTs | 3 |
| 401945 | c17p3_241 | | | 3 |
| 452102 | U04343 | Hs. 27954 | CD86 antigen (CD28 antigen ligand 2, B7- | 3 |
| 454292 | N57559 | Hs. 82273 | hypothetical protein | 3 |
| 402718 | c1p1_6495 | | | 3 |
| 441453 | AW176106 | Hs. 285459 | ESTs, Weakly similar to unknown [D. melan | 3 |
| 428785 | AI015953 | Hs. 125265 | ESTs | 3 |
| 414650 | AA150435 | Hs. 72063 | ESTs | 3 |
| 411173 | R81571 | | gb: yj02h10.r1 Soares placenta Nb2HP Homo | 3 |
| 446112 | AV656599 | Hs. 282636 | ESTs | 3 |
| 404439 | c8p1_195 | | | 3 |
| 412281 | AI810054 | Hs. 14119 | ESTs | 3 |
| 432965 | AW974144 | Hs. 133860 | ESTs | 3 |
| 404632 | c9p1_2941 | | | 3 |
| 459031 | AA017571 | Hs. 159398 | ESTs | 3 |
| 436195 | AA774834 | Hs. 75761 | SFRS protein kinase 1 | 3 |
| 428788 | AF082283 | Hs. 193516 | B-cell CLL/lymphoma 10 | 3 |
| 431512 | BE270734 | Hs. 2795 | lactate dehydrogenase A | 3 |
| 424536 | AW965002 | Hs. 47232 | ESTs | 3 |
| 443982 | AI222998 | Hs. 134962 | ESTs | 3 |
| 415120 | N64464 | Hs. 34950 | ESTs | 3 |
| 441555 | AI651563 | Hs. 178912 | ESTs | 3 |
| 419932 | AA281594 | | gb: zt03a01.r1 NCI_CGAP_GCB1 Homo sapiens | 3 |
| 422860 | S67798 | Hs. 121494 | sperm adhesion molecule 1 (PH-20 hyaluro | 3 |
| 453125 | AW779544 | Hs. 115497 | Homo sapiens cDNA: FLJ22655 fis, clone H | 3 |
| 448306 | AI480270 | | gb: tm26d07.x1 Soares_NFL_T_GBC_S1 Homo s | 3 |
| 406284 | AW068311 | Hs. 82582 | integrin, beta-like 1 (with EGF-like rep | 3 |
| 454289 | AL137554 | Hs. 49927 | Homo sapiens mRNA; cDNA DKFZp434H1720 (f | 3 |
| 403283 | c2p1_4629 | | | 3 |
| 439541 | AW970853 | | gb: EST382936 MAGE resequences, MAGK Homo | 4 |
| 431124 | AF284221 | Hs. 59506 | doublesex and mab-3 related transcriptio | 4 |
| 435701 | AW236397 | Hs. 63131 | Homo sapiens cDNA FLJ13155 fis, clone NT | 4 |
| 441577 | AI422096 | | gb: tf57h05.x1 NCI_CGAP_Brn23 Homo sapien | 4 |
| 429258 | AA448765 | | gb: zx10e09.r1 Soares_total_fetus_Nb2HF8_{—} | 4 |
| 432407 | AA221036 | Hs. 285026 | HERV-H LTR-associating 1 | 4 |
| 422017 | NM_003877 | Hs. 110776 | STAT induced STAT inhibitor-2 | 4 |
| 405591 | cNp3_27585 | | | 4 |
| 439824 | AW303556 | Hs. 124515 | ESTs | 4 |
| 456370 | AA234938 | Hs. 87384 | ESTs | 4 |
| 413539 | BE146879 | | gb: QV4-HT0222-261099-014-c11 HT0222 Homo | 4 |
| 409894 | BE081731 | | gb: QV2-BT0635-220400-158-e04 BT0635 Homo | 4 |
| 440344 | AA928516 | Hs. 190575 | ESTs | 4 |
| 445180 | BE217929 | Hs. 147470 | ESTs | 4 |
| 421089 | AB037771 | Hs. 101799 | KIAA1350 protein | 4 |
| 414118 | AI659167 | Hs. 75968 | thymosin, beta 4, X chromosome | 4 |
| 405863 | ph0_11166 | | | 4 |
| 432877 | AW974111 | Hs. 292477 | ESTs | 4 |
| 453043 | AW136440 | Hs. 224277 | ESTs | 4 |
| 443725 | AW245680 | Hs. 9701 | growth arrest and DNA-damage-inducible, | 4 |
| 411810 | AW947513 | | gb: RC0-MT0002-140300-011-e04 MT0002 Homo | 4 |
| 457807 | N89812 | Hs. 138809 | Human clone 23564 mRNA sequence | 4 |
| 444559 | W04370 | Hs. 282795 | ESTs | 4 |
| 401027 | c11p1_812 | | | 4 |
| 402134 | c19p1_1058 | | | 4 |
| 459646 | AW883968 | | gb: QV3-OT0063-290300-135-c04 OT0063 Homo | 4 |
| 420954 | AA282074 | Hs. 301753 | Homo sapiens cDNA FLJ11614 fis, clone HE | 4 |
| 425431 | T62818 | Hs. 257482 | ESTs | 4 |
| 446104 | AI571189 | Hs. 55977 | Homo sapiens cDNA: FLJ20985 fis, clone C | 4 |
| 410178 | AA082211 | Hs. 233936 | myosin, light polypeptide, regulatory, n | 4 |
| 441861 | AA970039 | Hs. 200940 | ESTs | 4 |
| 459045 | N69101 | Hs. 32703 | ESTs | 4 |
| 452948 | AW368451 | Hs. 188665 | ESTs, Weakly similar to sodium-hydrogen | 4 |
| 456375 | AF147766 | Hs. 199647 | Homo sapiens cDNA FLJ12993 fis, clone NT | 4 |
| 454365 | AW966728 | Hs. 54642 | methionine adenosyltransferase II, beta | 4 |
| 435864 | AL036499 | Hs. 188491 | ESTs | 4 |
| 428799 | AI478619 | Hs. 104677 | ESTs | 4 |
| 444268 | AI139642 | Hs. 143239 | ESTs | 4 |
| 457674 | AF119908 | Hs. 235516 | hypothetical protein PRO2955 | 4 |
| 434628 | H47495 | Hs. 13810 | ESTs | 4 |
| 459726 | AI904506 | | Homo sapiens cDNA: FLJ21802 fis, clone H | 4 |
| 438524 | AI824326 | Hs. 22305 | ESTs | 4 |
| 411280 | N50617 | | EST cluster (not in UniGene) | 4 |
| 431317 | AA502682 | | gb: ng23d01.s1 NCI_CGAP_Ov2 Homo sapiens | 4 |
| 454148 | AW732837 | Hs. 42390 | nasopharyngeal carcinoma susceptibility | 4 |
| 431723 | AW058350 | Hs. 16762 | Homo sapiens mRNA; cDNA DKFZp564B2062 (f | 4 |
| 418922 | AW956580 | Hs. 42699 | Thrombospondin-1 (Hs. 87409) | 4 |
| 426653 | AA530892 | Hs. 171695 | dual specificity phosphatase 1 | 4 |
| 458032 | AW979141 | Hs. 293917 | Homo sapiens cDNA FLJ11774 fis, clone HE | 4 |
| 412429 | AV650262 | Hs. 75765 | GRO2 oncogene | 4 |
| 421568 | W85858 | Hs. 99804 | ESTs, Weakly similar to ALU1_HUMAN ALU S | 4 |
| 413919 | BE180590 | | gb: RC3-HT0625-130400-021-d12 HT0625 Homo | 4 |
| 413576 | BE149684 | | gb: RC1-HT0256-280300-017-d10 HT0256 Homo | 4 |
| 454204 | AW816498 | | gb: QV0-ST0236-171299-075-b02 ST0236 Homo | 4 |
| 433712 | AF090887 | | gb: Homo sapiens clone HQ0085 | 4 |
| 445832 | AI261545 | | gb: qz30a07.x1 NCI_CGAP_Kid11 Homo sapien | 4 |
| 404432 | c8p1_1658 | | | 4 |
| 432745 | AI821926 | Hs. 269507 | ESTs | 4 |
| 405763 | cXp3_1388 | | | 4 |
| 416843 | D45467 | Hs. 58606 | ESTs | 4 |
| 427608 | BE148596 | Hs. 179779 | ribosomal protein L37 | 4 |
| 450594 | N31036 | | gb: yx51g04.r1 Soares melanocyte 2NbHM Ho | 4 |
| 427366 | AA885108 | Hs. 223806 | Homo sapiens cDNA: FLJ23157 fis, clone L | 4 |
| 445772 | AI733941 | Hs. 145493 | ESTs, Weakly similar to ALU7_HUMAN ALU S | 4 |
| 404485 | c8p1_4167 | | | 4 |
| 442621 | AI004333 | Hs. 130553 | ESTs, Weakly similar to ALUA_HUMAN !!!! | 4 |
| 410255 | AA234006 | Hs. 190488 | hypothetical protein FLJ10120 | 4 |
| 421358 | AA806749 | Hs. 290346 | ESTs | 4 |
| 416708 | H78836 | | gb: yu09a06.r1 Soares fetal liver spleen | 4 |
| 453434 | AJ271378 | Hs. 140951 | ESTs | 4 |
| 426946 | AA393595 | Hs. 97446 | ESTs | 4 |
| 446087 | AI298072 | Hs. 149441 | ESTs | 4 |
| 453816 | AL135405 | | gb: DKFZp762K1015_r1 762 (synonym: hmel2) | 4 |
| 448588 | AI970276 | Hs. 156905 | ESTs | 4 |
| 450775 | AA902384 | Hs. 110248 | ESTs | 4 |
| 416107 | AA173846 | Hs. 79015 | antigen identified by monoclonal antibod | 4 |
| 422766 | AA334108 | Hs. 159572 | heparan sulfate (glucosamine) 3-O-sulfot | 4 |
| 454991 | AW850163 | | gb: IL3-CT0219-271099-022-D02 CT0219 Homo | 4 |
| 407528 | X64990 | | gb: H. sapiens mRNA HTPCRX16 for olfactory | 4 |
| 441910 | AI150328 | Hs. 226402 | ESTs, Weakly similar to mitochondrial ci | 4 |
| 450438 | AI696071 | Hs. 253800 | ESTs | 4 |
| 430251 | AA609246 | Hs. 181451 | ESTs | 4 |
| 440358 | AW296778 | Hs. 300357 | ESTs, Highly similar to dJ416F21.2 [H.sa | 4 |
| 409236 | BE539805 | | gb: 601061906F1 NIH_MGC_10 Homo sapiens c | 4 |
| 449335 | AW150717 | Hs. 296176 | STAT induced STAT inhibitor 3 | 4 |
| 404099 | c6p1_1419 | | | 4 |
| 418214 | AA215293 | Hs. 156004 | ESTs | 4 |
| 406108 | ph0_2356 | | | 4 |
| 441492 | AI149998 | Hs. 146346 | ESTs | 4 |
| 458867 | AW995393 | | gb: QV0-BN0042-170300-163-g12 BN0042 Homo | 4 |
| 447283 | BE061049 | Hs. 258396 | ESTs | 4 |
| 401514 | AF147186 | | gb: AF147186 Homo sapiens library (Schere | 4 |
| 428263 | AA424811 | Hs. 152155 | ESTs | 4 |
| 434970 | AW272262 | Hs. 250468 | ESTs | 4 |
| 440882 | AI205777 | Hs. 129538 | ESTs | 4 |
| 405099 | cNp1_23324 | | | 4 |
| 458960 | BE383204 | | gb: 601298758F1 NIH_MGC_19 Homo sapiens c | 4 |
| 453611 | BE009728 | | gb: PM0-BN0173-120400-001-f09 BN0173 Homo | 4 |
| 447605 | AW504937 | Hs. 211169 | ESTs | 4 |
| 436819 | AA731746 | Hs. 120232 | ESTs | 4 |
| 419134 | T89863 | Hs. 221771 | ESTs | 4 |
| 403159 | c2p1_1584 | | | 4 |
| 456973 | AA375710 | Hs. 102746 | ESTs | 4 |
| 401454 | c14p3_4909 | | | 4 |
| 412625 | AA114946 | Hs. 261314 | ESTs | 4 |
| 417551 | AI816291 | Hs. 82273 | hypothetical protein | 4 |
| 440962 | AI989961 | Hs. 233477 | ESTs, Moderately similar to A Chain A, S | 4 |
| 423291 | NM_004129 | Hs. 126590 | guanylate cyclase 1, soluble, beta 2 | 5 |
| 417977 | AA210787 | Hs. 243748 | ESTs | 5 |
| 456667 | AW665591 | Hs. 114658 | ESTs | 5 |
| 402337 | c19p3_4189 | | | 5 |
| 451838 | AW005866 | Hs. 193969 | ESTs | 5 |
| 406346 | ph2_13138 | | | 5 |
| 404121 | c6p1_3615 | | | 5 |
| 446698 | AW451812 | Hs. 202503 | ESTs | 5 |
| 444988 | AF272830 | Hs. 12229 | Homo sapiens cDNA FLJ11324 fis, clone PL | 5 |
| 418443 | NM_005239 | Hs. 85146 | v-ets avian erythroblastosis virus E26 o | 5 |
| 457993 | AI799102 | Hs. 292732 | ESTs, Weakly similar to Gab2 [H. sapiens] | 5 |
| 446546 | BE167687 | Hs. 156628 | ESTs | 5 |
| 435917 | AA702143 | Hs. 190365 | ESTs | 5 |
| 413496 | BE144841 | | gb: CM0-HT0181-181099-075-f10 HT0181 Homo | 5 |
| 403661 | c3p3_219 | | | 5 |
| 421177 | AW070211 | Hs. 102415 | Homo sapiens mRNA; cDNA DKFZp586N0121 (f | 5 |
| 459660 | M79082 | | ESTs | 5 |
| 411708 | AW857808 | | gb: RC4-CT0322-261299-011-c02 CT0322 Homo | 5 |
| 411962 | AA099050 | | gb: zk85d12.r1 Soares_pregnant_uterus_NbH | 5 |
| 426087 | M61877 | Hs. 1985 | spectrin. alpha, erythrocytic 1 (ellipto | 5 |
| 416188 | BE157260 | Hs. 79070 | v-myc avian myelocytomatosis viral oncog | 5 |
| 458410 | H20380 | Hs. 200250 | ESTs, Weakly similar to neuronal thread | 5 |
| 405387 | cNp3_16477 | | | 5 |
| 457873 | AA736920 | Hs. 288518 | ESTs | 5 |
| 453511 | AL031224 | Hs. 33102 | transcription factor AP-2 beta (activati | 5 |
| 438863 | R38002 | | gb: yh97g12.r1 Soares placenta Nb2HP Homo | 5 |
| 458467 | AW747996 | Hs. 160999 | ESTs | 5 |
| 447844 | AI433873 | Hs. 35085 | ESTs | 5 |
| 438675 | AA813725 | Hs. 213568 | ESTs | 5 |
| 439170 | AA332365 | Hs. 165539 | ESTs | 5 |
| 410483 | BE163567 | | gb: QV3-HT0460-230200-101-b08 HT0460 Homo | 5 |
| 401276 | c13p1_293 | | | 5 |
| 430725 | AA485056 | Hs. 173692 | ESTs | 5 |
| 449834 | AL161980 | Hs. 24022 | Homo sapiens mRNA; cDNA DKFZp761H1023 (f | 5 |
| 400827 | c11p1_19707 | | | 5 |
| 425534 | AA995635 | Hs. 7589 | ESTs | 5 |
| 416493 | H60593 | Hs. 124990 | ESTs | 5 |
| 434977 | AI734233 | Hs. 226142 | ESTs, Weakly similar to ALU7_HUMAN ALU S | 5 |
| 456466 | AA700127 | Hs. 190504 | ESTs | 5 |
| 458489 | AI142274 | Hs. 145423 | ESTs | 5 |
| 433232 | AI658621 | Hs. 127769 | ESTs | 5 |
| 420270 | AA257990 | | gb: zs35h07.r1 NcI_CGAP_GCB1 Homo sapiens | 5 |
| 454410 | AW812744 | | gb: RC3-ST0186-181099-012-c09 ST0186 Homo | 5 |
| 420431 | AB007131 | Hs. 97624 | heat shock transcription factor 2 bindin | 5 |
| 400844 | c11p1_20936 | | | 5 |
| 435517 | AA928626 | Hs. 130177 | ESTs | 5 |
| 412996 | BE046224 | | gb: hn38c12.x2 NCI_CGAP_RDF2 Homo sapiens | 5 |
| 420475 | AW408407 | Hs. 187018 | ESTs | 5 |
| 421126 | M74587 | Hs. 102122 | insulin-like growth factor binding prote | 5 |
| 430269 | BE221682 | Hs. 178364 | ESTs | 5 |
| 425673 | R70318 | | gb: yj81a09.r1 Soares breast 2NbHBst Homo | 5 |
| 435057 | AW291345 | Hs. 254970 | ESTs | 5 |
| 405667 | cNp3_6851 | | | 5 |
| 402527 | c1p1_18184 | | | 6 |
| 459407 | N92114 | | gb: za22h11.r1 Soares fetal liver spleen | 6 |
| 454302 | AA306105 | Hs. 50785 | SEC22, vesicle trafficking protein (S. c | 6 |
| 426436 | AA378512 | Hs. 287639 | Homo sapiens cDNA FLJ14334 fis, clone PL | 6 |
| 412791 | AI131192 | Hs. 143199 | ESTs | 6 |
| 441189 | AW450266 | Hs. 257276 | ESTs | 6 |
| 456013 | T92048 | | gb: yd54g12.s1 Soares fetal liver spleen | 6 |
| 441698 | BE299588 | Hs. 28465 | Homo sapiens cDNA: FLJ21869 fis, clone H | 6 |
| 438597 | AA811662 | Hs. 171497 | ESTs | 6 |
| 456592 | R91600 | | gb: yq10c02.r1 Soares fetal liver spleen | 6 |
| 402312 | c19p3_3548 | | | 6 |
| 418912 | NM_000685 | Hs. 89472 | angiotensin receptor 1 | 6 |
| 440700 | AW952281 | Hs. 296184 | ESTs, Highly similar to GB01_HUMAN GUANI | 6 |
| 424128 | AW966163 | | gb: EST378236 MAGE resequences, MAGI Homo | 7 |
| 419711 | C02621 | Hs. 159282 | ESTs | 7 |
| 444851 | AL117425 | Hs. 301413 | Homo sapiens cDNA FLJ11516 fis, clone HE | 7 |
| 414137 | BE220829 | Hs. 50652 | ESTs, Moderately similar toALU1_HUMAN A | 7 |
| 401438 | c14p3_401 | | | 7 |
| 457656 | AA625087 | Hs. 224405 | ESTs | 7 |
| 434402 | AA745143 | Hs. 212498 | ESTs | 7 |
| 413574 | BE149158 | Hs. 129998 | Homo sapiens cDNA FLJ14267 fis, clone PL | 8 |
| 447317 | BE312948 | Hs. 18104 | hypothetical protein FLJ11274 | 8 |
| 444698 | AI188139 | Hs. 147050 | ESTs | 12 |
| 430758 | T91568 | Hs. 270616 | ESTs, Moderately similar to A34087 hypot | 12 |
| 452049 | BE268289 | Hs. 27693 | CGI-124 protein | 19 |

Pkey: Unique Eos probeset identifier number
ExAccn: Exemplar Accession number, Genbank accession number
UnigeneID: Unigene number
Title: Gene title
R1: Non-diseased liver: Diseased Liver

TABLE 2B

| | | |
|---|---|---|
| 451400 | 1103195_1 | BE069211 BE160479 BE160478 AI793147 AW861059 |
| 415328 | 1870242_1 | Z44310 R55952 F05790 |
| 408834 | 717496_1 | AW276241 BE167263 BE167259 |
| 455806 | 1515155_1 | BE141094 BE141097 BE141263 BE141118 BE141253 BE141256 BE141225 BE141092 BE141124 BE141248 BE141254 |
| | | BE141262 BE141093 |
| | | BE141115 BE141259 BE141255 BE141226 BE141213 BE141222 BE141128 BE141129 BE141211 BE141157 |
| | | BE141257 BE141258 BE141250 |
| | | BE141116 BE141208 BE141227 BE141096 BE141112 BE141261 BE141121 BE141120 BE141300 BE141111 |
| | | BE141117 BE141131 BE141212 |
| | | BE141223 BE141090 |
| 455181 | 1106814_1 | BE161696 AW863568 BE161629 BE161824 |
| 423167 | 879906_1 | BG995664 AA322711 D79268 AA770464 |
| 459263 | 2049982_1 | AJ003496 AJ003505 L25475 |
| 455175 | 1103799_1 | AW993247 AW861464 |
| 454448 | 1028414_1 | BE073941 BE073901 AW750209 |
| 409113 | 49403_1 | AF319957 AA113914 AA131489 AA113889 AA075684 AA071047 AA126078 AA126283 AA075895 AA074583 |
| | | AA070580 AA131372 AA079230 |
| | | AA148748 AA120938 AA079200 AA122355 AA075041 AA071086 AA071110 AA074485 AA076151 AA070940 AA071308 |
| | | AA070627 AA076622 |
| | | AA079623 AA078802 AA079143 AA085188 AA079434 AA075968 AA071453 AA074159 AA076131 AA079401 AA115163 |
| | | AA079329 AA069053 |
| | | AA101144 AA070053 AA071087 AA102076 AA071310 AA122204 AA079659 AA063317 AA148628 AA078803 AA121103 |
| | | AA076187 AA078931 |
| | | AA070156 AA074198 AA070928 AA127089 AA083070 AA079280 AA134725 AA126185 AA076056 AA078833 AA084710 |
| | | AA079117 AA084027 |
| | | AA075042 AA129030 AA068994 AA069817 AA074897 AA079208 AA085044 AA081472 AA069220 AA071430 AA085118 |
| | | AA083166 AA079450 |
| | | AA074563 AA065051 AA100188 AA115929 AA064871 AA129031 |
| 459689 | 1272662_1 | AA347192 |
| 439034 | 52270_1 | AF075083 H52291 H52528 |
| 415350 | 1870780_1 | T80268 R18726 Z44987 F12665 F11445 F11572 F06404 T74313 R13218 R13622 |
| 459695 | 817414_1 | AW963571 AA381579 BG547915 H69131 |
| 455195 | 1107903_1 | AW864319 AW864370 AW864504 |
| 411861 | 223814_1 | BE067343 AW867875 BE067301 BE067347 BE067303 BE067304 BF851070 BE067350 BE067305 BE067306 |
| | | BE067302 AW938147 |
| 456576 | 267535_1 | AA287443 AI478347 AA419385 BE084078 |
| 454442 | 44916_2 | AL529783 BF804681 BI858809 AW748795 AW816134 BE063456 BI911312 BG615273 AA347409 |
| 454520 | 825_6 | BC020822 AW803378 AW803435 AW803371 BI518461 AV762185 AA298048 BI521095 AV764359 AV760834 |
| | | BG548457 BI911189 |
| 459349 | 1027822_1 | AW749381 H93337 |
| 455104 | 1096744_1 | BF330730 BF350539 BE153665 BE065062 BE064650 BE064863 BF330763 BE153820 BE064737 BE155079 |
| | | BE064651 AW856751 AW856622 |
| | | BE064691 BE153674 BE153698 BE064730 BE153536 |
| 424200 | 890908_1 | AW966196 AA337221 AA336756 |
| 423290 | 7964_7 | AJ295991 AU138209 AV649543 BG195745 BG208133 AI458145 AW183395 AV649405 BF950906 AW962011 |
| | | AW902934 AA324130 BI753806 |
| 449305 | 1066708_1 | AW813561 AI638293 |
| 413257 | 1497012_1 | BE075035 BE074999 BE075006 BE075008 BE075005 BE075032 BE075037 |
| 430692 | 60836_7 | AI133594 AI064750 AW328184 |
| 424686 | 1228447_1 | AW963243 AA345251 AA345504 |
| 411658 | 1095903_1 | AW855645 AW855615 AW855610 BE148763 BE148764 BG951004 BG950922 BG950984 BG950998 BG950924 |
| | | BG950921 BG951005 BG950995 |
| | | BG950988 BG950919 AW855605 AW855608 BG950991 AW855598 AW855601 AW855596 BG950985 |
| 459721 | 36709_2 | BE256910 AI299050 BG471673 |
| 442484 | 3699_10 | AI110863 AF075362 AI110861 AF075360 |
| 411689 | 1097490_1 | AW857121 AW861238 AW857123 |
| 411902 | 1141058_1 | AW875344 AW875287 AW875285 AW875286 BF361295 AW875402 AW875400 |
| 418225 | 1239780_1 | AW976061 AA747676 AA214595 |
| 410900 | 1063481_1 | AW810169 AW809654 AW809839 AW810090 AW809703 AW809891 BF374636 BF374628 BF374725 AW810616 |
| | | AW809733 BF374640 BF374623 |
| | | AW810564 |
| 449986 | 2292_10 | AK055879 AW007836 AA873089 N74374 AV720071 AA702706 AW055276 BE672779 AW864502 AI678780 AW864369 |
| | | AI052145 T40984 N74426 |
| 413088 | 1489839_1 | BE064856 BE064853 BE064960 BE064962 BE064857 BE064977 BE064860 BE064850 BE064815 BE064816 |
| | | BE064806 BE064818 BE064796 |
| | | BE064804 BE064668 BE064810 BE064979 BE064957 BE064819 BE064975 BE065059 |
| 448156 | 515877_1 | BI495496 AW023207 BI495497 AI472886 AW023239 |
| 454573 | 22511_7 | AW833609 AW833743 BI035140 AW821469 AW821541 AW821488 AW821531 AW821384 AW821625 AW821547 |
| | | AW821549 AW821513 |
| | | AW821577 BI034572 BI034365 |
| 433391 | 16854_1 | AF038194 BG431979 BF987851 R52301 T77201 |
| 449637 | 34908_4 | AL558074 BI914921 AV747407 AV749215 AA001964 |
| 436857 | 448721_1 | AA732647 BE009028 BE008970 |
| 411484 | 1085933_1 | AW848117 AW848128 AW848278 AW848401 AW848405 AW848281 AW848763 |
| 453493 | 470764_1 | AL039478 AW970882 |
| 408766 | 106215_1 | BG028106 BE155008 BE155007 AA057270 AA058715 |
| 455036 | 1091116_1 | AW851708 AW851735 AW851703 AW851630 AW851712 AW851723 |
| 421036 | 264886_1 | AW977543 AA282918 |
| 455404 | 703567_1 | BE175503 BE175583 AW936409 AW936404 |
| 453502 | 1668_5 | AF332192 NM_032491 BG772831 BG772873 BI460034 BI560011 BG723202 BG701021 BG720172 BI826825 |
| | | BI464806 BG723004 BI559763 |
| | | BI460842 BG773364 BI460208 BF377004 AL042201 BE855589 BE857075 AL044045 AA906504 AA961631 |
| | | AW899993 AA885061 AW900006 |
| | | AI652619 AI002078 AA983818 BG152590 BE763010 Z38801 F04136 F02320 H17003 BE762943 AI191585 N66165 |
| | | BE762995 Z40423 AL039786 |
| | | BI559354 BI562672 BG718512 BG720380 BI458642 BG772693 BI561299 BI459216 BF933814 R13208 Z42633 F07285 |
| | | H10144 Z42852 F07873 |
| | | R35183 H17002 F07362 BI459457 AL041856 BG724404 BG718143 BG701878 R50843 H10145 R40295 AW592045 |
| | | BE935655 BG771671 |
| 409854 | 916262_1 | AW502145 AW501833 AW502581 |
| 454754 | 1070974_1 | AW819177 AW819242 AW819191 AW819175 AW819252 AW819244 AW819265 AW819269 AW819190 |
| | | AW819268 AW819183 AW819246 |
| | | AW819194 AW819249 AW819186 AW819180 AW819188 BE158470 AW819251 BE152602 AW819263 |
| 419392 | 215562_2 | W28573 W27418 |
| 454803 | 1072913_1 | BI468492 AW862380 AW860148 AW821887 AW821863 AW821894 AW821870 AW862378 AW862351 |
| 439884 | 10759_1 | AB051476 BG289143 AV753375 AW051603 AW294678 AI435358 AI357776 AI091413 AI435427 AI367010 AI538999 |
| | | AI039731 N67220 AW119213 |
| | | BE326750 AI369016 T16459 R55315 AW296026 AI553628 AI537645 AI923565 BE910660 AW237341 H42671 |
| | | H09709 AA847991 AA355842 |
| | | BM454895 Z46035 AW195056 D63011 AI765593 BF999411 R55417 N91158 H88285 H99837 H49679 D61792 |
| | | H52824 AW603615 R33635 |
| | | D29082 |
| 454816 | 1073598_1 | AW833298 AW833294 AW833272 AW833271 AW833274 AW833308 AW833275 AW833296 AW833295 |
| | | AW833258 AW833306 |
| 437208 | 28382_1 | AL110259 AA236599 |
| 412234 | 1160035_1 | AW902569 AW902557 AW902654 AW902641 AW902650 AW902741 AW902644 |
| 410158 | 114375_1 | BI256712 BF327164 AW936396 AW936458 AW936418 BF380035 BF368137 |
| 459472 | 1095076_1 | AW854431 AA568933 |
| 410815 | 3326_23 | AW805981 AW805974 AW806135 AW805972 |
| 436681 | 6833_1 | AK021878 AU119692 AW502159 AU145969 AI393268 BE939969 AI288242 AI051978 BE677884 AI436745 |
| | | AI935582 AI686473 AW861386 |
| | | AA725270 BE940025 BF829279 |
| 436317 | 3614_2 | AL096777 BM457842 AU116901 NM_021946 AK021424 AU158712 BE328021 BF765194 BF804830 BE621537 AA968672 |
| 443338 | 2262575_1 | R99575 AI052252 R99681 |
| 416659 | 290122_1 | W22048 W19418 H72518 BF908045 |
| 411173 | 881247_1 | AW962014 AA324277 BI022237 AA091723 R81571 |
| 419932 | 3433_3 | BI766402 AI365043 AA251996 N46090 AA281594 |
| 448306 | 50570_1 | AK057289 AL601840 AL602227 BE247000 AA417150 BM150327 BM151615 BE247403 BE245780 AI480270 |
| | | BE246663 BE245079 H54482 |
| | | AI004637 T97117 AU157626 BE243570 AA781826 AA418396 H82937 |
| 439541 | 1235485_1 | AW970853 N22817 AA837349 |
| 441577 | 17241_1 | AK055721 AA399241 AI204074 AW269179 AI150462 AI422096 AA938959 AA024620 AA024498 AI751426 |
| 429258 | 121944_1 | BG250865 AA448765 AA658293 C04967 BG988507 BG746352 C03045 |
| 413539 | 1519622_1 | BE146879 BE146914 BE146918 |
| 409894 | 919627_1 | AW503629 AW861738 BE081731 BE081969 |
| 411810 | 1107901_1 | AW947513 AW864536 AW864318 |
| 459646 | 154497_1 | AW470813 H44995 AW883968 BF746199 BF746344 BF746274 BF511374 |
| 459726 | 15924_1 | NM_024644 AK025455 BC011350 AI240194 AA576870 AW295198 AW262665 AA968435 AA815311 AW769847 |
| | | AA100496 AI796246 BI257802 |
| | | AI968266 BF447872 AW341239 BE674505 AI183838 BF221583 Z40969 N64168 AI904509 AI904506 BG610839 |
| | | BG107618 BG108325 BE784665 |
| | | BE389806 BE390268 BF000100 AW444473 BF194857 BE843654 AW449497 BE093686 AL523620 AW402647 |
| | | BI753888 BI913225 BM040984 |
| | | Z45254 BF794833 BI915966 BI831226 AL523619 BG991161 BG957808 BE895148 BG469054 |
| 411280 | 1610_5 | BF527858 AV713798 N50617 N47321 BF871615 R54159 BF741988 BF741990 BF741989 AW860545 AW835317 |
| 431317 | 997174_1 | AW970601 AW613399 AA503435 AA502682 N91138 |
| 413919 | 1565718_1 | BE180590 BE180585 BE180594 |
| 413576 | 417218_1 | BG535869 BM263801 AV703254 T39786 BM263489 BE149684 T39845 |
| 454204 | 646158_1 | AW816498 BF374419 BF374408 BF374405 AW808977 AW808605 BF334681 BF348941 BF348944 AW178676 BF374412 |
| | | AW178486 BF374427 |
| | | BF374429 BF348942 BF374428 BF348955 BF348940 BF348943 BF374416 BF374424 BF374431 BF374430 AW808524 |
| | | BF374413 BF374418 |
| | | BF334708 BF374389 BF334685 BF374473 BF374392 BF374397 BF374395 BF374407 BF374417 BF374420 BF374414 |
| | | BF374422 BF374421 |
| | | BF374522 BF349306 AW808532 BF374399 BF374393 BF374398 BF374394 BF374396 AW178485 BF374391 |
| | | AW808816 BF374516 AW178483 |
| | | AW808515 AW808791 BF374390 BF374415 AW808514 AW808379 BF374423 BF374426 BF348949 AW809007 |
| 433712 | 77122_3 | AF090887 AI110655 AF063529 |
| 445832 | 437253_1 | BF116098 AI261545 AW875247 N59134 AW875371 |
| 450594 | 82197_1 | N31036 N42915 F07753 AA010329 |
| 416708 | 309677_1 | R07686 T95204 T95230 H78836 BF932909 |
| 453816 | 9500_19 | BG107738 BE149281 AL135405 AW891435 |
| 454991 | 1088900_1 | AW850659 AW850532 AW850667 BE143543 AW850163 BF367228 AW850661 |
| 409236 | 777089_1 | BE539805 BE536062 AW368376 |
| 458867 | 1246993_1 | AW995393 AJ403118 |
| 458960 | 50855_1 | NM_024331 BC003071 BM012414 BE315221 BG750119 BE272198 AL449476 BE886722 AI360302 BG002949 |
| | | BM454474 AL449598 BM012506 |
| | | BE383204 AA010225 AL449685 AL449684 BF742320 T06328 |
| 453611 | 1477969_1 | AL045316 BF009728 |
| 413496 | 1517996_1 | BE144708 BE144844 BE144705 BE144828 BE144815 BE144701 BE144841 BF144823 BE144836 |
| 459660 | 25501_86 | BE072622 M79082 |
| 411708 | 1098544_1 | AW857808 AW857817 AW857833 AW857837 AW857873 |
| 411962 | 2307710_1 | AA099050 AA099526 T47733 |
| 438863 | 52130_1 | AF075004 BF109017 R38002 R38003 F22027 |
| 410483 | 1028391_1 | BE073747 AW750178 BE163567 BE073739 BE073748 BE073780 BE163491 BE163495 BE073763 BE073671 |
| | | BE073689 BE073769 |
| 420270 | 258327_1 | AW816460 AA257990 AI416981 AW500873 |
| 454410 | 6852_9 | AW812744 AW581974 BG985054 AW812725 |
| 412996 | 1343199_1 | BE046224 BE046730 BE046302 |
| 425673 | 727129_1 | BG697146 AA361514 AW957439 AW298175 BI495720 R70319 AA579358 AI798179 AI633067 BG743245 |
| | | AW403725 T49604 R70318 |
| 459407 | 1990083_1 | H82757 N92114 |
| 456013 | 46281_8 | BC015430 BG033733 T92048 |
| 456592 | 268841_1 | AA291455 R91600 T87079 |
| 424128 | 890041_1 | AW966163 AA335983 AA335973 AA336011 AA335668 |

Pkey: Unique Eos probeset identifier number
CAT number: Gene cluster number
Accession: Genbank accession numbers

TABLE 2C

| Pkey | Ref | strand | Nt_position |
|---|---|---|---|
| 404839 | 7109502 | Minus | 11386-11689 |
| 400959 | 7705148 | Minus | 129453-130097 |
| 403397 | 9438368 | Minus | 84481-84655 |
| 404723 | 9884767 | Minus | 22795-22968 |
| 404398 | 9802820 | Minus | 17421-17497, 26796-26954, |
| | | | 30866-30974 |
| 405475 | 1931025 | Plus | 1548-1702 |
| 404498 | 8151654 | Plus | 13292-13497 |
| 401074 | 3687273 | Plus | 72667-72812 |
| 401887 | 7229981 | Plus | 93973-94120 |
| 406547 | 7711513 | Minus | 172780-174358 |
| 402468 | 9797107 | Minus | 23969-24933 |
| 405278 | 6139075 | Minus | 3863-3965, 4823-4891, 5439-5529, |
| | | | 6043-6170 |
| 405456 | 7656676 | Plus | 150052-150208 |
| 404319 | 9211467 | Plus | 54436-54608 |
| 403600 | 8101279 | Minus | 3680-3838 |
| 401775 | 9966311 | Minus | 110228-110340 |
| 405532 | 9755485 | Minus | 37485-39417 |
| 401411 | 7799787 | Minus | 144144-144329 |
| 403212 | 7630897 | Minus | 156037-156210 |
| 404834 | 6911603 | Minus | 37948-38226 |
| 401866 | 8018106 | Plus | 73126-73623 |
| 401558 | 7139678 | Plus | 103510-104090 |
| 405698 | 4165331 | Plus | 54114-54225 |
| 404974 | 3241949 | Minus | 12524-13612 |
| 404196 | 3805917 | Minus | 67928-68109 |
| 401758 | 9910067 | Minus | 146471-147987 |
| 401946 | 4914397 | Plus | 85670-85752, 86415-86571, |
| | | | 87635-87796, 8791 |
| 402627 | 9931216 | Plus | 12136-12272, 16487-16628, |
| | | | 17654-17798, 1849 |
| 406562 | 7711584 | Plus | 37316-37426 |
| 402747 | 9212492 | Minus | 7105-7357 |
| 400488 | 8919452 | Plus | 97365-98784 |
| 406225 | 7417725 | Minus | 9581-10055 |
| 406531 | 7711474 | Minus | 20515-20648, 22519-22601 |
| 405548 | 1532158 | Plus | 11552-11686 |
| 405510 | 7630909 | Minus | 101028-101174 |
| 403003 | 5441423 | Minus | 79403-79560, 79712-80021 |
| 404499 | 8151657 | Plus | 19376-19909 |
| 401945 | 4914397 | Plus | 83342-83809 |
| 402718 | 8969253 | Plus | 102033-102302, 103219-103485 |
| 404439 | 7139680 | Plus | 55316-55585 |
| 404632 | 9796668 | Plus | 45096-45229 |
| 403283 | 8076905 | Minus | 71124-71996 |
| 405591 | 6960456 | Plus | 146384-146641, 147035-147160 |
| 405863 | 7657810 | Plus | 49410-49620 |
| 401027 | 7230983 | Minus | 70407-70554, 71060-71160 |
| 402134 | 7704979 | Minus | 108621-109936 |
| 404432 | 7407979 | Minus | 123536-123660 |
| 405763 | 5931935 | Plus | 274920-275019, 276641-276802 |
| 404485 | 8096921 | Plus | 75166-75264, 124036-124232 |
| 404099 | 8076888 | Minus | 127375-127477 |
| 406108 | 7107999 | Plus | 89468-89674 |
| 401514 | 7622355 | Plus | 93224-93292, 94913-95065, |
| | | | 95163-95334 |
| 405099 | 8074292 | Minus | 114365-114514, 128635-128831 |
| 403159 | 7408087 | Plus | 2775-2977 |
| 401454 | 9186923 | Minus | 114659-114832 |
| 402337 | 6957691 | Plus | 4116-4286, 16811-16973, |
| | | | 17107-17256, 19715- |
| 406346 | 9255974 | Plus | 104359-104542 |
| 404121 | 9796219 | Plus | 59256-59401 |
| 403661 | 8705027 | Minus | 30268-30482 |
| 405387 | 6587915 | Minus | 3769-3833, 5708-5895 |
| 401276 | 8954274 | Minus | 15919-16096 |
| 400827 | 8570385 | Plus | 143937-144450 |
| 400844 | 9188605 | Plus | 24746-24872, 25035-25204 |
| 405667 | 4726099 | Plus | 5798-5914 |
| 402527 | 9800806 | Plus | 4722-4916, 17858-18037, |
| | | | 19964-20140, 24423- |
| 402312 | 7341442 | Plus | 143645-143727, 147428-147514 |
| 401438 | 4885691 | Minus | 72461-72605 |

Pkey: Unique number corresponding to an Eos probeset
Ref: Sequence source. The 7 digit numbers in this column are Genbank Identifier (GI) numbers. “Dunham, et al.” refers to the publication entitled “The DNA sequence of human chromosome 22” Dunham, et al. (1999) Nature 402: 489-495.
Strand: Indicates DNA strand from which exons were predicted.
Nt_position: Indicates nucleotide positions of predicted exons.

Tables 3A to 6C [see 60/366,782].

Table 3A lists about 1389 genes up-regulated in Hepatitis C positive liver tissues compared to Hepatitis C negative liver tissues. These were selected from 59680 probesets on the Affymetrix/Eos Hu03 GeneChip array such that the Wicoxon rank-sum test p-value between the 2 groups was less than 0.10, the ratio of the “weighted average” of Hepatitis C positive liver tissues to the “weighted average” of Hepatitis C negative liver tissues was equal to or above 2.0, and that the differences between the same 2 groups was equal to or above 30.0. The “weighted average” of the Hepatitis C positive liver tissues was set to the trimean of various different Hepatitis C positive liver tissues. The “weighted average” of the Hepatitis C negative liver tissues was set to the either 10 or the trimean of various different Hepatitis C negative liver tissues, whichever value was greater to eliminate ratios with a denominator of zero or less.

TABLE 3A
ABOUT 1389 GENES UP-REGULATED IN HEPATITIS C POSITIVE LIVER TISSUES
COMPARED TO HEPATITIS C NEGATIVE LIVER TISSUES

| Pkey | ExAccn | UnigeneID | Unigene Title | R1 | R2 | R3 |
|---|---|---|---|---|---|---|
| 428227 | AA321649 | Hs. 2248 | small inducible cytokine subfamily B (Cy | 5.544E−08 | 25.27 | 278.25 |
| 414004 | AA737033 | Hs. 7155 | ESTs, Moderately similar to 2115357A TYK | 1.131E−07 | 24.52 | 252.25 |
| 417621 | AV654694 | Hs. 82316 | interferon-induced, hepatitis C-associat | 8.497E−08 | 23.13 | 243 |
| 422746 | NM_004484 | Hs. 119651 | glypican 3 | 8.647E−07 | 22.55 | 255.75 |
| 426711 | AA383471 | Hs. 343800 | conserved gene amplified in osteosarcoma | 5.544E−08 | 20.97 | 224.5 |
| 433854 | AA610649 | Hs. 333239 | ESTs | 1.322E−05 | 19.88 | 191 |
| 424090 | X99699 | Hs. 139262 | XIAP associated factor-1 | 9.289E−06 | 17.80 | 190 |
| 448111 | AA053486 | Hs. 20315 | interferon-induced protein with tetratri | 2.262E−07 | 15.74 | 224.75 |
| 451652 | AA018968 | Hs. 133536 | ESTs | 1.321E−05 | 15.05 | 149.25 |
| 427283 | AL119796 | Hs. 174185 | ectonucleotide pyrophosphatase/phosphodi | 1.646E−06 | 14.90 | 189.75 |
| 418216 | AA662240 | Hs. 283099 | AF15q14 protein | 1.494E−07 | 14.17 | 153.25 |
| 432094 | AI658580 | Hs. 61426 | Homo sapiens mesenchymal stem cell prote | 1.322E−05 | 13.72 | 128.25 |
| 425787 | AA363867 | Hs. 155029 | ESTs | 5.734E−06 | 13.57 | 151.75 |
| 430200 | BE613337 | Hs. 234896 | geminin | 1.492E−07 | 13.25 | 122.5 |
| 446094 | AK001760 | Hs. 13801 | KIAA1685 protein | 5.391E−06 | 12.90 | 138.5 |
| 413670 | AB000115 | Hs. 75470 | hypothetical protein, expressed in osteo | 3.643E−07 | 12.25 | 211 |
| 450937 | R49131 | Hs. 26267 | ATP-dependant interferon response protei | 8.647E−07 | 12.07 | 117 |
| 442048 | AA974603 | | gb: op34f05.s1 Soares_NFL_T_GBC_S1 Homo s | 1.481E−05 | 11.92 | 115 |
| 408393 | AW015318 | Hs. 23165 | ESTs | 2.127E−06 | 11.82 | 130.75 |
| 415443 | T07353 | Hs. 7948 | ESTs | 2.129E−04 | 11.80 | 115.75 |
| 417308 | H60720 | Hs. 81892 | KIAA0101 gene product | 6.467E−06 | 11.70 | 118.75 |
| 416206 | AW206248 | Hs. 111092 | hypothetical protein FLJ22332 | 5.441E−07 | 11.35 | 135.25 |
| 432378 | AI493046 | Hs. 146133 | ESTs | 1.450E−06 | 11.13 | 107.5 |
| 445823 | AI478563 | Hs. 145519 | FKSG87 protein | 3.961E−06 | 11.00 | 101.25 |
| 447973 | AB011169 | Hs. 20141 | similar to S. cerevisiae SSM4 | 2.487E−05 | 10.97 | 99.75 |
| 409153 | W03754 | Hs. 50813 | hypothetical protein FLJ20022 | 2.972E−07 | 10.72 | 118.75 |
| 414052 | AW578849 | Hs. 283552 | ESTs, Weakly similar to unnamed protein | 8.090E−07 | 10.45 | 142.25 |
| 409231 | AA446644 | Hs. 692 | GA733-2 antigen; epithelial glycoprotein | 1.871E−05 | 10.30 | 130.75 |
| 407347 | AA829847 | | gb: od40d07.s1 NCI_CGAP_GCB1 Homo sapiens | 2.904E−06 | 10.05 | 93 |
| 403790 | | | NM_001334*: Homo sapiens cathepsin O (CTS | 1.765E−03 | 9.65 | 103.25 |
| 450293 | N36754 | Hs. 171118 | hypothetical protein FLJ00026 | 7.923E−05 | 9.56 | 117.75 |
| 421057 | T58283 | | Homo sapiens cDNA: FLJ22063 fis, clone H | 2.601E−03 | 9.27 | 85 |
| 428065 | AI634046 | Hs. 157313 | ESTs | 6.525E−04 | 9.22 | 93.25 |
| 418318 | U47732 | Hs. 84072 | transmembrane 4 superfamily member 3 | 9.302E−05 | 9.21 | 115 |
| 451752 | AB032997 | Hs. 26966 | KIAA1171 protein | 6.383E−08 | 9.20 | 112.75 |
| 444665 | BE613126 | Hs. 47783 | B aggressive lymphoma gene | 1.641E−06 | 9.17 | 82.25 |
| 435812 | AA700439 | Hs. 188490 | ESTs | 2.485E−05 | 9.13 | 101.75 |
| 418143 | AA283057 | Hs. 266957 | hypothetical protein FLJ14281 | 2.732E−04 | 9.00 | 82.5 |
| 435665 | AI248952 | Hs. 12320 | ESTs | 9.212E−07 | 8.85 | 103 |
| 421282 | AA286914 | Hs. 183299 | ESTs | 3.862E−04 | 8.67 | 102.5 |
| 426793 | X89887 | Hs. 172350 | HIR (histone cell cycle regulation defec | 1.083E−03 | 8.63 | 86.25 |
| 407644 | D16815 | Hs. 37288 | nuclear receptor subfamily 1, group D, m | 2.737E−04 | 8.56 | 115.25 |
| 422546 | AB007969 | Hs. 301478 | KIAA0500 protein | 2.408E−06 | 8.52 | 81.25 |
| 429490 | AI971131 | Hs. 23889 | ESTs, Weakly similar to ALU7_HUMAN ALU S | 2.951E−05 | 8.50 | 117 |
| 442994 | AI026718 | Hs. 16954 | ESTs | 5.152E−04 | 8.47 | 93.25 |
| 416224 | NM_02902 | Hs. 79088 | reticulocalbin 2, EF-hand calcium bindin | 7.048E−07 | 8.45 | 90.75 |
| 405102 | | | C15001220*: gi\|4469558\|gb\|AAD21311.1\|(AF | 1.038E−03 | 8.20 | 84.5 |
| 444314 | AI140497 | | gb: ow76b09.s1 Soares_fetal_liver_spleen_{—} | 4.679E−04 | 8.10 | 81.25 |
| 426818 | AA554827 | Hs. 340046 | DKFZp434A0131 protein | 1.358E−03 | 8.02 | 86.5 |
| 417933 | X02308 | Hs. 82962 | thymidylate synthetase | 6.228E−04 | 7.95 | 73 |
| 432954 | AI076345 | Hs. 214199 | ESTs | 1.567E−04 | 7.88 | 76.5 |
| 449541 | AA730673 | Hs. 188634 | ESTs | 3.295E−05 | 7.85 | 70 |
| 410315 | AI638871 | Hs. 17625 | Homo sapiens cDNA: FLJ22524 fis, clone H | 1.985E−06 | 7.80 | 86 |
| 446619 | AU076643 | Hs. 313 | secreted phosphoprotein 1 (osteopontin, | 3.481E−03 | 7.80 | 83.25 |
| 433586 | T85301 | Hs. 194397 | gb: yd78d06.s1 Soares fetal liver spleen | 1.417E−03 | 7.80 | 75 |
| 436995 | AI160015 | Hs. 118112 | ESTs | 3.858E−04 | 7.80 | 71 |
| 437374 | AL359571 | Hs. 44054 | ninein (GSK3B interacting protein) | 9.840E−06 | 7.70 | 77 |
| 421904 | BE143533 | Hs. 109309 | hypothetical protein FLJ20035 | 2.972E−07 | 7.67 | 130.5 |
| 442679 | R53718 | Hs. 107882 | hypothetical protein FLJ10659 | 1.048E−06 | 7.65 | 113 |
| 433401 | AF039698 | Hs. 284217 | serologically defined colon cancer antig | 1.113E−06 | 7.55 | 83 |
| 435571 | AF212225 | Hs. 283693 | mitochondrial ribosomal protein L1 | 1.315E−05 | 7.55 | 79.25 |
| 419175 | AW270037 | Hs. 179507 | KIAA0779 protein | 7.276E−06 | 7.50 | 65 |
| 407930 | AA045847 | Hs. 188361 | Homo sapiens cDNA FLJ12807 fis, clone NT | 5.420E−05 | 7.47 | 92.25 |
| 434263 | N34895 | Hs. 44648 | ESTs | 2.099E−03 | 7.47 | 67.5 |
| 448481 | W15284 | Hs. 74832 | ESTs | 7.876E−04 | 7.45 | 66.25 |
| 449935 | AA004798 | Hs. 108311 | ESTs, Weakly similar to T00351 hypotheti | 7.510E−05 | 7.38 | 79 |
| 407949 | W21874 | Hs. 247057 | ESTs, Weakly similar to 2109260A B cell | 7.276E−06 | 7.34 | 126.75 |
| 435102 | AW899053 | Hs. 76917 | F-box only protein 8 | 6.222E−04 | 7.32 | 75.25 |
| 418452 | BE379749 | Hs. 85201 | C-type (calcium dependent, carbohydrate- | 2.417E−07 | 7.32 | 99.5 |
| 425053 | AF046024 | Hs. 154320 | ubiquitin-activating enzyme E1C (homolog | 7.505E−05 | 7.30 | 73.75 |
| 436090 | AI640635 | Hs. 332879 | EST | 6.091E−03 | 7.29 | 81.75 |
| 458725 | AW970192 | Hs. 171942 | ras responsive element binding protein 1 | 6.048E−05 | 7.25 | 92.25 |
| 446488 | AB037782 | Hs. 15119 | KIAA1361 protein | 2.738E−04 | 7.25 | 88.25 |
| 449718 | AA459480 | Hs. 23956 | hypothetical protein FLJ20502 | 1.994E−06 | 7.17 | 86 |
| 418840 | AI821614 | Hs. 185831 | ESTs | 5.649E−03 | 7.10 | 72.75 |
| 408063 | BE086548 | Hs. 42346 | calcineurin-binding protein calsarcin-1 | 4.354E−05 | 7.05 | 100.5 |
| 436024 | AI800041 | Hs. 190555 | ESTs | 4.030E−04 | 7.02 | 72.75 |
| 447574 | AF162666 | Hs. 18895 | tousled-like kinase 1 | 3.903E−05 | 6.97 | 88.75 |
| 447809 | AW207605 | Hs. 164230 | ESTs, Highly similar to JC7266 3′,5′-cyc | 1.547E−06 | 6.97 | 87 |
| 424878 | H57111 | Hs. 221132 | ESTs | 2.240E−04 | 6.95 | 105.25 |
| 450016 | AA249590 | Hs. 100748 | ESTs, Weakly similar to A28996 prolinE-r | 8.224E−06 | 6.90 | 63.75 |
| 452744 | AI267652 | Hs. 246107 | Homo sapiens mRNA; cDNA DKFZp434E082 (fr | 1.969E−07 | 6.82 | 237 |
| 433230 | AW136134 | Hs. 220277 | ESTs | 1.176E−05 | 6.80 | 99.75 |
| 409052 | AW898179 | Hs. 50123 | zinc finger protein 189 | 3.075E−03 | 6.80 | 59.75 |
| 434280 | BE005398 | | gb: CM1-BN0116-150400-189-h02 BN0116 Homo | 1.650E−04 | 6.79 | 81 |
| 419586 | AI088485 | Hs. 144759 | ESTs, Weakly similar to I38022 hypotheti | 5.435E−07 | 6.77 | 76.25 |
| 422506 | R20909 | Hs. 300741 | sorcin | 1.488E−02 | 6.77 | 70.75 |
| 424941 | AA128376 | Hs. 153884 | ATP binding protein associated with cell | 5.724E−05 | 6.77 | 62 |
| 411605 | AW006831 | Hs. 145409 | ESTs | 2.596E−03 | 6.76 | 89.25 |
| 433208 | AW002834 | Hs. 24095 | ESTs | 7.123E−03 | 6.75 | 65.5 |
| 433730 | AK002135 | Hs. 3542 | hypothetical protein FLJ11273 | 9.472E−04 | 6.72 | 86.5 |
| 445929 | AI089660 | Hs. 323401 | dpy-30-like protein | 1.828E−04 | 6.72 | 74.75 |
| 448694 | AA478756 | Hs. 194477 | E3 ubiquitin ligase SMURF2 | 1.210E−04 | 6.71 | 88.5 |
| 446667 | BE161878 | Hs. 224805 | ESTs | 4.102E−03 | 6.70 | 64.25 |
| 452973 | H88409 | Hs. 93788 | ESTs | 9.463E−04 | 6.70 | 63.25 |
| 405268 | | | ENSP00000223174*: KIAA0783 PROTEIN. | 1.274E−04 | 6.70 | 60 |
| 423732 | AF058056 | Hs. 132183 | solute carrier family 16 (monocarboxylic | 4.464E−04 | 6.67 | 64 |
| 431214 | AA294921 | Hs. 348024 | v-ral simian leukemia viral oncogene horn | 2.707E−03 | 6.67 | 65.25 |
| 419407 | AW410377 | Hs. 41502 | hypothetical protein FLJ21276 | 2.125E−06 | 6.60 | 103.75 |
| 422040 | AA172106 | Hs. 110950 | Rag C protein | 1.652E−04 | 6.58 | 60 |
| 443547 | AW271273 | Hs. 23767 | hypothetical protein FLJ12666 | 2.117E−06 | 6.57 | 95 |
| 446493 | AK001389 | Hs. 15144 | hypothetical protein DKFZp564O043 | 2.734E−04 | 6.55 | 80.25 |
| 416475 | T70298 | | gb: yd26g02.s1 Soares fetal liver spleen | 1.091E−04 | 6.55 | 73 |
| 447541 | AK000288 | Hs. 18800 | hypothetical protein FLJ20281 | 2.282E−03 | 6.55 | 63.75 |
| 441976 | AA428403 | Hs. 106131 | ESTs | 1.082E−04 | 6.55 | 62.75 |
| 434375 | BE277910 | Hs. 3833 | 3′-phosphoadenosine 5′-phosphosulfate sy | 4.776E−06 | 6.52 | 80 |
| 408096 | BE250162 | Hs. 83765 | dihydrofolate reductase | 3.206E−03 | 6.52 | 71.25 |
| 453887 | BE564037 | Hs. 36237 | hypothetical protein | 5.688E−05 | 6.52 | 62.75 |
| 419550 | D50918 | Hs. 90998 | KIAA0128 protein; septin 2 | 3.397E−07 | 6.50 | 71 |
| 416133 | NM_001683 | Hs. 89512 | ATPase, Ca transporting, plasma membrane | 3.206E−03 | 6.45 | 60.5 |
| 442993 | BE018682 | Hs. 166196 | ATPase, Class I, type 8B, member 1 | 6.863E−06 | 6.40 | 77.5 |
| 413645 | AA130992 | | gb: zo15e02.s1 Stratagene colon (937204) | 4.820E−03 | 6.36 | 60.25 |
| 445525 | BE149866 | Hs. 14831 | Homo sapiens, Similar to zinc finger pro | 1.120E−06 | 6.35 | 74.5 |
| 431183 | NM_006855 | Hs. 250696 | KDEL (Lys-Asp-Glu-Leu) endoplasmic relic | 8.296E−03 | 6.35 | 61.5 |
| 434474 | AL042936 | Hs. 211571 | holocytocbrome c synthase (cytochrome c | 6.345E−03 | 6.32 | 56 |
| 435029 | AF167706 | Hs. 19280 | cysteine-rich motor neuron 1 | 5.425E−03 | 6.32 | 53.5 |
| 409512 | AW979187 | Hs. 293591 | melanoma differentiation associated prot | 7.934E−08 | 6.27 | 221.5 |
| 426925 | NM_001196 | Hs. 315689 | Homo sapiens cDNA: FLJ22373 fis. clone H | 8363E−05 | 6.25 | 68.25 |
| 418476 | AA648431 | Hs. 37883 | hypothetical protein PNAS-131 | 9.290E−05 | 6.25 | 65 |
| 421097 | AI280112 | Hs. 125232 | Homo sapiens cDNA FLJ13266 fis, clone OV | 1.543E−02 | 6.25 | 53.5 |
| 414405 | AI362533 | Hs. 306117 | KIAA0306 protein | 5.377E−06 | 6.23 | 68 |
| 451788 | BE242857 | Hs. 27021 | hypothetical protein FLJ11159 | 3.020E−04 | 6.22 | 62 |
| 408753 | AI337192 | Hs. 47438 | SH3 domain binding glutamic acid-rich pr | 1.764E−03 | 6.22 | 54 |
| 442045 | C05768 | Hs. 8078 | Homo sapiens clone FBD3 Cri-du-chat crit | 2.024E−04 | 6.19 | 67.5 |
| 414183 | AW957446 | Hs. 301711 | ESTs | 2.024E−04 | 6.17 | 74.5 |
| 425073 | W39609 | Hs. 22003 | solute carrier family 6 (neurotransmitte | 1.838E−03 | 6.17 | 54.5 |
| 426110 | NM_002913 | Hs. 166563 | replication factor C (activator 1)1(14 | 8.722E−06 | 6.13 | 65 |
| 435511 | AA683336 | Hs. 189046 | ESTs | 3.204E−03 | 6.11 | 56.25 |
| 407218 | AA095473 | Hs. 28505 | ubiquitin-conjugating enzyme E2H (homolo | 6.093E−03 | 6.10 | 66.25 |
| 422173 | BE385828 | Hs. 250619 | phorbolin-like protein MDS019 | 2.582E−07 | 6.05 | 104.5 |
| 443852 | AI679966 | Hs. 150603 | ESTs | 1.692E−03 | 6.02 | 71.25 |
| 445813 | Z42023 | Hs. 106576 | alaninE-glyoxylate aminotransferase 2-li | 4.050E−04 | 6.02 | 66 |
| 413922 | AI535895 | Hs. 221024 | ESTs | 3.628E−03 | 6.00 | 78.75 |
| 440624 | AF017987 | Hs. 7306 | secreted frizzled-related protein 1 | 2.595E−03 | 6.00 | 60 |
| 426458 | D83032 | Hs. 169984 | nuclear protein | 2.601E−03 | 6.00 | 57.25 |
| 418791 | AA935633 | Hs. 194628 | ESTs | 4.684E−04 | 5.99 | 89.75 |
| 437629 | AW574774 | Hs. 121692 | ESTs | 1.485E−04 | 5.98 | 51 |
| 447164 | AF026941 | Hs. 17518 | Homo sapiens cig5 mRNA, partial sequence | 2.959E−07 | 5.97 | 145.25 |
| 424699 | AW206227 | Hs. 287727 | hypothetical protein FLJ23132 | 6.525E−04 | 5.97 | 54.25 |
| 443035 | Z45822 | Hs. 8906 | Homo sapiens clone 24889 mRNA sequence | 1.271E−04 | 5.97 | 50.75 |
| 452827 | AI571835 | Hs. 55468 | ESTs | 9.250E−06 | 5.96 | 103 |
| 431604 | AF175265 | Hs. 264190 | vacuolar protein sorting 35 (yeast homol | 2.024E−04 | 5.95 | 62.75 |
| 445943 | AW898533 | Hs. 181574 | ESTs | 3.331E−04 | 5.95 | 60.75 |
| 451122 | AA015767 | Hs. 84522 | ESTs | 4.624E−03 | 5.95 | 53 |
| 425167 | AA351629 | Hs. 225567 | ESTs | 2.237E−04 | 5.93 | 53 |
| 411252 | AB018549 | Hs. 69328 | MD-2 protein | 8.196E−06 | 5.92 | 103.75 |
| 423598 | BE247600 | Hs. 155538 | ESTs | 6.835E−04 | 5.92 | 51 |
| 431736 | AI912234 | Hs. 3297 | ribosomal protein S27a | 3.326E−04 | 5.91 | 83.5 |
| 410361 | BE391804 | Hs. 62661 | guanylate binding protein 1, interferon- | 5.384E−04 | 5.88 | 86 |
| 417228 | AL134324 | Hs. 7312 | ESTs | 2.953E−05 | 5.88 | 63 |
| 415938 | BE383507 | Hs. 78921 | A kinase (PRKA) anchor protein 1 | 2.218E−05 | 5.83 | 77.25 |
| 427484 | N32859 | Hs. 37288 | nuclear receptor subfamily 1. group D, m | 5.068E−06 | 5.82 | 114 |
| 443291 | AA325633 | Hs. 136102 | KIAA0853 protein | 3.022E−04 | 5.82 | 78.5 |
| 424308 | AW975531 | Hs. 154443 | minichramosome maintenance deficient (S. | 4.611E−05 | 5.82 | 60 |
| 430261 | AA305127 | Hs. 237225 | hypothetical protein HT023 | 7.140E−04 | 5.82 | 58.5 |
| 447735 | AA775268 | Hs. 6127 | Homo sapiens cDNA: FLJ23020 fis, clone L | 5.114E−05 | 5.82 | 48.75 |
| 419644 | AU076951 | Hs. 91797 | retinoblastoma-binding protein 1 | 9.265E−06 | 5.80 | 70.75 |
| 456619 | AV647917 | Hs. 107153 | inhibitor of growth family, member 1-lik | 2.219E−05 | 5.80 | 67.25 |
| 414812 | X72755 | Hs. 77367 | monokine induced by gamma interferon | 3.893E 07 | 5.78 | 181.75 |
| 418876 | AA740616 | | gb: ny97f11.s1 NCI_CGAP_GCB1 Homo sapiens | 1.042E−05 | 5.75 | 77.5 |
| 408360 | AI806090 | Hs. 44344 | hypothetical protein FLJ20534 | 1.103E−05 | 5.75 | 59.25 |
| 418224 | AL036057 | Hs. 83795 | interferon regulatory factor 2 | 1.482E−04 | 5.75 | 58.25 |
| 456236 | AF045229 | Hs. 82280 | regulator of G-protein signalling 10 | 3.860E−04 | 5.72 | 54.25 |
| 449609 | BE246434 | Hs. 289026 | guanine nucleotide binding protein (G pr | 3.658E−04 | 5.72 | 49 |
| 448554 | NM_016169 | Hs. 21431 | suppressor of fused | 7.897E−05 | 5.72 | 49 |
| 445529 | H14421 | Hs. 180513 | ATP-binding cassette, sub-family A (ABC1 | 7.515E−05 | 5.70 | 61.75 |
| 434210 | AA665612 | Hs. 90093 | ESTs | 1.287E−07 | 5.67 | 99.5 |
| 410577 | X91911 | Hs. 64639 | glioma pathogenesis-related protein | 1.113E−06 | 5.67 | 95 |
| 400517 | AF242388 | | lengsin | 9.250E−06 | 5.67 | 73.5 |
| 438011 | BE466173 | Hs. 145696 | splicing factor (CC1.3) | 8.617E−04 | 5.67 | 64.25 |
| 456439 | AA251242 | Hs. 103238 | ESTs | 2.830E−03 | 5.67 | 48 |
| 421594 | R45689 | Hs. 21889 | Homo sapiens cDNA FLJ12978 fis, clone NT | 3.342E−03 | 5.65 | 48.75 |
| 408214 | AL120445 | Hs. 77823 | hypothetical protein FLJ21343 | 4.253E−04 | 5.60 | 58 |
| 441028 | AI333660 | Hs. 17558 | Homo sapiens cDNA FLJ14446 fis, clone HE | 8.358E−05 | 5.59 | 58.5 |
| 421650 | AA781795 | Hs. 122587 | ESTs | 3.287E−06 | 5.57 | 95.75 |
| 433037 | NM_014158 | Hs. 279938 | HSPC067 protein | 9.795E−05 | 5.57 | 69.75 |
| 409277 | T05558 | Hs. 156880 | ESTs | 1.084E−03 | 5.57 | 62.5 |
| 428172 | U09367 | Hs. 182828 | zinc finger protein 136 (clone pHZ-20) | 1.490E−04 | 5.57 | 48.5 |
| 424915 | R42755 | Hs. 23096 | ESTs | 1.565E−04 | 5.54 | 46.5 |
| 431863 | AA188185 | Hs. 289043 | spindlin | 9.265E−06 | 5.53 | 148.5 |
| 457130 | NM_005651 | Hs. 183671 | tryptophan 2,3-dioxygenase | 3.622E−03 | 5.52 | 79 |
| 431122 | AI267593 | Hs. 250535 | Homo sapiens mRNA; cDNA DKFZp434N2412 (f | 3.670E−04 | 5.52 | 47.25 |
| 413509 | BE145419 | | gb: IL5-HT0198-291099-009-E01 HT0198 Homo | 7.118E−03 | 5.52 | 46.75 |
| 412802 | U41518 | Hs. 74602 | aquaporin 1 (channel-forming integral pr | 1.275E−04 | 5.50 | 95.5 |
| 416309 | R84694 | Hs. 79194 | cAMP responsive element binding protein | 4.750E−06 | 5.50 | 60.5 |
| 437730 | AW071087 | Hs. 239176 | insulin-like growth factor 1 receptor | 7.833E−04 | 5.50 | 48.5 |
| 410867 | X63556 | Hs. 750 | fibrillin 1 (Marfan syndrome) | 1.438E−02 | 5.50 | 48.25 |
| 431586 | AW971100 | Hs. 293189 | ESTs | 3.341E−03 | 5.47 | 55.75 |
| 420520 | AK001978 | Hs. 98510 | similar to rab11-binding protein | 2.701E−03 | 5.47 | 55.25 |
| 421633 | AF121860 | Hs. 106260 | sorting nexin 10 | 1.201E−02 | 5.47 | 52.5 |
| 437175 | AW968078 | Hs. 87773 | protein kinase, cAMP-dependent, catalyti | 1.735E−04 | 5.46 | 62.5 |
| 428467 | AK002121 | Hs. 184465 | hypothetical protein FLJ11259 | 5.146E−04 | 5.45 | 46.75 |
| 443441 | AW291196 | Hs. 92195 | ESTs | 3.555E−02 | 5.45 | 50.5 |
| 424243 | AI949359 | Hs. 339739 | ESTs, Highly similar to cis Golgi-locali | 5.416E−05 | 5.45 | 49.75 |
| 415660 | AI909007 | Hs. 78563 | ubiquitin-conjugating enzyme E2G 1 (homo | 1.596E−02 | 5.45 | 48 |
| 439008 | AF075072 | Hs. 167535 | ESTs, Weakly similar to ALU1_HUMAN ALU S | 1.297E−03 | 5.45 | 47 |
| 420789 | AI670057 | Hs. 199882 | ESTs | 4.814E−03 | 5.45 | 46.75 |
| 432388 | X15218 | Hs. 2969 | v-ski avian sarcoma viral oncogene homol | 2.734E−04 | 5.43 | 46.5 |
| 439375 | AA689526 | Hs. 344249 | steroid dehydrogenase homolog | 2.219E−05 | 542 | 64.75 |
| 413010 | AA393273 | Hs. 75133 | transcription factor 6-like 1 (mitochond | 1.846E−03 | 5.42 | 46.5 |
| 448901 | AK001021 | Hs. 22505 | hypothetical protein FLJ10159 | 1.989E−06 | 5.40 | 76.75 |
| 429747 | M87507 | Hs. 2490 | caspase 1, apoptosis-related cysteine pr | 9.219E−06 | 5.40 | 69.5 |
| 453742 | AB037744 | Hs. 34892 | KIAA1323 protein | 7.173E−04 | 5.40 | 55.5 |
| 405141 | Y14443 | | zinc finger protein 200 | 1.159E−02 | 5.40 | 44.5 |
| 450206 | AI796450 | Hs. 201600 | ESTs | 2.735E−04 | 5.38 | 47 |
| 422553 | AI697720 | Hs. 171455 | ESTs, Weakly similar to T31613 hypotheti | 6.857E−06 | 5.35 | 84 |
| 408048 | NM_007203 | Hs. 42322 | A kinase (PRKA) anchor protein 2 | 1.240E−03 | 5.35 | 58.75 |
| 420338 | AA825595 | Hs. 88269 | Homo sapiens, clone MGC: 17339, mRNA, com | 2.123E−06 | 5.32 | 49.5 |
| 414279 | AW021691 | | GCN5 (general control of ammo-acid synt | 1.544E−02 | 5.30 | 56 |
| 449429 | AA054224 | Hs. 59847 | ESTs | 3.076E−03 | 5.30 | 52 |
| 417688 | R09170 | Hs. 284350 | ESTs | 1.487E−04 | 5.30 | 43.25 |
| 434421 | AI915927 | Hs. 34771 | ESTs | 4.454E−04 | 5.30 | 65.5 |
| 439301 | AA833784 | Hs. 252888 | ESTs | 1.085E−03 | 5.27 | 74.5 |
| 433505 | AW504027 | Hs. 15301 | Homo sapiens cDNA FLJ12596 fis, clone NT | 6.228E−04 | 5.27 | 51.25 |
| 430556 | AW967807 | Hs. 13797 | ESTs | 1.295E−03 | 5.26 | 72.5 |
| 414737 | AI160386 | Hs. 125087 | ESTs | 2.395E−06 | 5.25 | 108.25 |
| 439971 | W32474 | Hs. 301746 | RAP2A, member of RAS oncogene family | 1.753E−06 | 5.25 | 86.75 |
| 450697 | AW152166 | Hs. 182113 | ESTs | 8.621E−04 | 5.25 | 68 |
| 419490 | NM_006144 | Hs. 90708 | granzyme A (granzyme 1, cytotoxic T-lymp | 3.208E−03 | 5.25 | 55.5 |
| 433001 | AF217513 | Hs. 279905 | clone HQ0310PRO0310p1 | 1.345E−04 | 5.23 | 87.75 |
| 432441 | AW292425 | Hs. 163484 | ESTs | 2.471E−04 | 5.22 | 76 |
| 449618 | AI076459 | Hs. 15978 | KIAA1272 protein | 9.832E−02 | 5.22 | 46.75 |
| 433437 | U20536 | Hs. 3280 | caspase 6, apoptosis-related cysteine pr | 1.649E−06 | 5.20 | 92.5 |
| 450916 | AA011597 | Hs. 177398 | ESTs | 7.511E−04 | 5.20 | 94.5 |
| 427699 | AW965076 | Hs. 180378 | hypothetical protein 669 | 1.928E−03 | 5.20 | 53.25 |
| 421535 | AB002359 | Hs. 105478 | phosphoribosylformylglycinamidinesyntha | 6.512E−04 | 5.20 | 44.25 |
| 410511 | AA743475 | Hs. 285655 | ESTs | 2.947E−03 | 5.20 | 43 |
| 448760 | AA313825 | Hs. 21941 | AD036 protein | 2.628E−05 | 5.19 | 66 |
| 452327 | AK000196 | Hs. 29052 | hypothetical protein FLJ20189 | 5.424E−05 | 5.17 | 58.25 |
| 449365 | AW968261 | Hs. 118913 | ESTs, Moderately similar to T46371 hypot | 4.097E−03 | 5.17 | 53.75 |
| 413007 | BE046662 | | gb: hn42f02.x1 NCI_CGAP_RDF2 Homo sapiens | 4.681E−04 | 5.17 | 44.5 |
| 414522 | AW518944 | Hs. 76325 | step II splicing factor SLU7 | 4.684E−04 | 5.16 | 139.25 |
| 451081 | AI078645 | Hs. 431 | murine leukemia viral (bmi-1) oncogene h | 2.490E−03 | 5.15 | 49.25 |
| 437834 | AA769294 | Hs. 283854 | gb: nz36g03.s1 NCI_CGAP_GCB1 Homo sapiens | 3.307E−04 | 5.13 | 55.5 |
| 458965 | AA010319 | Hs. 60389 | ESTs | 3.856E−04 | 5.13 | 41.75 |
| 442878 | AI868648 | Hs. 22315 | ESTs | 3.672E−04 | 5.10 | 58.75 |
| 410337 | M83822 | Hs. 62354 | cell division cycle 4-like | 1.652E−04 | 5.10 | 55.75 |
| 422267 | AB033044 | Hs. 114012 | KIAA1218 protein | 1.405E−04 | 5.10 | 47.25 |
| 438865 | H64256 | Hs. 167619 | ESTs, Moderately similar to ALUC_HUMAN! | 3.034E−02 | 5.10 | 42.5 |
| 438914 | N93892 | Hs. 10727 | ESTs | 5.630E−03 | 5.10 | 43 |
| 407204 | R41933 | Hs. 140237 | ESTs, Weakly similar to ALU1_HUMAN ALU S | 1.663E−05 | 5.07 | 61.5 |
| 431049 | AA846576 | Hs. 103267 | hypothetical protein FLJ22548 similar to | 7.687E−06 | 5.07 | 55.5 |
| 417355 | D13168 | Hs. 82002 | endothelin receptor type B | 5.142E−02 | 5.07 | 45.25 |
| 419522 | AI682428 | Hs. 157728 | ESTs | 8.292E−05 | 5.05 | 48.25 |
| 433697 | AA600357 | Hs. 239489 | TIA1 cytotoxic granule-associated RNA-bi | 5.421E−03 | 5.02 | 48.25 |
| 428420 | AL096858 | Hs. 184245 | KIAA0929 protein Msx2 interacting nuclea | 7.583E−07 | 5.01 | 128.25 |
| 434658 | AI624436 | Hs. 310286 | ESTs | 6.092E−06 | 5.00 | 113.25 |
| 430268 | AK000737 | Hs. 237480 | hypothetical protein FLJ20730 | 1.480E−03 | 5.00 | 50.75 |
| 426052 | N49068 | Hs. 93966 | ESTs | 7.107E−05 | 5.00 | 47.25 |
| 450086 | AW016343 | Hs. 233301 | ESTs | 2.491E−03 | 5.00 | 47 |
| 430512 | AF182294 | Hs. 241578 | U6 snRNA-associated Sm-like protein LSm8 | 1.038E−02 | 5.00 | 44.5 |
| 456034 | AW450979 | | gb: UI-H-BI3-ata-a-12-0-Ul.s1 NCI_CGAP_Su | 9.642E−03 | 5.00 | 40.75 |
| 432606 | NM_002104 | Hs. 3066 | granzyme K (serine protease, granzyme 3; | 2.094E−05 | 4.99 | 146.5 |
| 431620 | AA126109 | Hs. 264981 | 2′-5′-oligoadenylate synthetase 2 (69-71 | 7.389E−08 | 4.98 | 241.75 |
| 440043 | BE277457 | Hs. 30661 | hypothetical protein MGC4606 | 1.297E−03 | 4.97 | 43.25 |
| 427008 | Z45258 | Hs. 286013 | short coiled-coil protein | 2.785E−05 | 4.97 | 68.5 |
| 443884 | N20617 | Hs. 194397 | leptin receptor | 4.448E−03 | 4.95 | 74.25 |
| 444670 | H58373 | Hs. 332938 | hypothetical protein MGC5370 | 3.017E−04 | 4.95 | 65 |
| 424852 | AI222779 | Hs. 144848 | ESTs | 6.341E−03 | 4.95 | 48 |
| 450747 | AI064821 | Hs. 318535 | ESTs, Highly similar to 1818357A EWS gen | 3.670E−02 | 4.95 | 45 |
| 411360 | AK001601 | Hs. 69594 | high-mobility group 20A | 5.639E−03 | 4.95 | 45 |
| 407366 | AF026942 | Hs. 271530 | gb: Homo sapiens cig33 mRNA, partial sequ | 8.647E−07 | 4.92 | 115.5 |
| 422150 | AI867118 | Hs. 279607 | calpastatin | 1.437E−02 | 4.92 | 42 |
| 419135 | R61448 | Hs. 106728 | ESTs, Weakly similar to KIAA1353 protein | 5.724E−06 | 4.92 | 115.75 |
| 414646 | AA353776 | Hs. 901 | CD48 antigen (B-cell membrane protein) | 2.413E−06 | 4.90 | 87.5 |
| 450447 | AF212223 | Hs. 25010 | hypothetical protein P15-2 | 4.456E−04 | 4.90 | 54 |
| 434158 | T86534 | Hs. 14372 | ESTs | 2.190E−03 | 4.90 | 46.75 |
| 420151 | AA255931 | Hs. 186704 | ESTs | 2.209E−05 | 4.90 | 46.75 |
| 408831 | AF090114 | Hs. 48433 | endocrine regulator | 1.732E−04 | 4.90 | 41.25 |
| 449500 | AW956345 | Hs. 12926 | ESTs | 2.839E−02 | 4.90 | 40.75 |
| 418662 | AI801098 | Hs. 151500 | ESTs | 1.716E−02 | 4.88 | 49.25 |
| 416050 | U51903 | Hs. 78993 | IQ motif containing GTPase activating pr | 2.108E−02 | 4.88 | 43 |
| 451338 | AW612322 | Hs. 19131 | transcription factor Dp-2 (E2F dimerizat | 2.730E−04 | 4.86 | 40.5 |
| 449523 | NM_000579 | Hs. 54443 | chemokine (C-C motif) receptor 5 | 1.871E−05 | 4.85 | 59.5 |
| 423857 | N48902 | Hs. 133481 | Homo sapiens mRNA; cDNA DKFZp564O0862 (f | 1.764E−03 | 4.82 | 58.75 |
| 427384 | T82854 | | gb: yd42a09.r1 Soares fetal liver spleen | 2.180E−02 | 4.82 | 43.75 |
| 451273 | NM_014811 | Hs. 26163 | KIAA0649 gene product | 9.414E−04 | 4.82 | 38.25 |
| 456508 | AA502764 | Hs. 123469 | ESTs, Weakly similar to AF208855 1 BM-01 | 1.298E−03 | 4.81 | 55.25 |
| 457584 | AA147979 | Hs. 285005 | mitochondrial import receptor Tom22 | 5.139E−05 | 4.80 | 57 |
| 443998 | AI620661 | Hs. 296276 | ESTs | 2.290E−03 | 4.77 | 77 |
| 454075 | R43826 | Hs. 16313 | Kruppel-like zinc finger protein GLIS2 | 2.100E−03 | 4.77 | 58.5 |
| 407609 | R43159 | Hs. 238432 | ESTs | 9.450E−04 | 4.77 | 54.75 |
| 424683 | N87519 | Hs. 27196 | ESTs | 1.650E−04 | 4.77 | 52.5 |
| 452820 | N46161 | Hs. 35274 | ESTs | 2.103E−03 | 4.77 | 46.75 |
| 441866 | BE464341 | Hs. 21201 | nectin 3; DKFZP566B0846 protein | 3.134E−02 | 4.77 | 40.5 |
| 429966 | BE081342 | Hs. 283037 | HSPC039 protein | 5.784E−02 | 4.77 | 39 |
| 449613 | N63808 | Hs. 34299 | ESTs | 2.725E−06 | 4.75 | 102 |
| 409703 | NM_006187 | Hs. 56009 | 2′-5′-oligoadenylate synthetase 3 (100 k | 5.544E−08 | 4.75 | 441 |
| 439334 | AI148976 | Hs. 112062 | ESTs | 3.154E−04 | 4.74 | 40.25 |
| 410382 | AW664971 | Hs. 259546 | ESTs | 1.207E−04 | 4.72 | 57.75 |
| 434926 | BE543269 | Hs. 50252 | mitochondrial ribosomal protein L32 | 1.733E−04 | 4.72 | 57 |
| 433198 | AA992841 | Hs. 27263 | KIAA1458 protein | 1.339E−02 | 4.72 | 44 |
| 435970 | H75410 | Hs. 54452 | zinc finger protein, subfamily 1 A, 1 (Ik | 5.395E−04 | 4.70 | 46.25 |
| 416647 | BE297139 | Hs. 79411 | replication protein A2 (32 kD) | 3.670E−04 | 4.68 | 66.25 |
| 409038 | T97490 | Hs. 50002 | small inducible cytokine subfamily A (Cy | 2.243E−04 | 4.68 | 193 |
| 429952 | AF080158 | Hs. 226573 | inhibitor of kappa light polypeptide gen | 3.684E−05 | 4.67 | 67.5 |
| 413048 | M93221 | Hs. 75182 | mannose receptor, C type 1 | 7.864E−04 | 4.67 | 52.25 |
| 425068 | AL048716 | Hs. 154387 | KIAA0103 gene product | 2.843E−02 | 4.67 | 40.75 |
| 450401 | AW959281 | Hs. 8184 | ESTs | 1.547E−03 | 4.67 | 39.75 |
| 402727 | | | NM_025065: Homo sapiens hypothetical prot | 1.118E−02 | 4.67 | 55 |
| 449209 | BE616830 | Hs. 294145 | ESTs | 3.170E−04 | 4.66 | 58.5 |
| 432600 | AI821085 | | gb: ns95a12.y5 NCI_CGAP_Pr3 Homo sapiens | 2.022E−04 | 4.65 | 84.25 |
| 441892 | AB028981 | Hs. 8021 | KIAA1058 protein | 1.867E−05 | 4.65 | 55.5 |
| 407284 | AI539227 | Hs. 214039 | hypothetical protein FLJ23556 | 8.131E−02 | 4.65 | 51.75 |
| 450516 | AA902656 | Hs. 21943 | NIF3 (Ngg1 interacting factor 3, S. pombe | 3.658E−04 | 4.65 | 49 |
| 426728 | NM_007118 | Hs. 171957 | triple functional domain (PTPRF interact | 6.500E−02 | 4.64 | 41 |
| 449909 | AA004681 | Hs. 59432 | ESTs | 1.767E−03 | 4.63 | 37.25 |
| 409401 | AI201895 | Hs. 181309 | proteasome (prosome, macropain) subunit, | 4.052E−04 | 4.63 | 61 |
| 450669 | AL138077 | Hs. 16157 | hypothetical protein FLJ12707 | 7.864E−04 | 4.63 | 36.25 |
| 408405 | AK001332 | Hs. 44672 | hypothetical protein FLJ10470 | 3.393E−07 | 4.61 | 115.5 |
| 451079 | AI827988 | Hs. 240728 | ESTs, Moderately similar to PC4259 ferri | 8.821E−05 | 4.60 | 60.5 |
| 408108 | AI580492 | Hs. 42743 | hypothetical protein | 5.663E−04 | 4.60 | 47.75 |
| 453555 | N23574 | Hs. 123649 | ESTs, Moderately similar to ALU7_HUMAN A | 2.764E−05 | 4.60 | 40.25 |
| 437967 | BE277414 | Hs. 5947 | mel transforming oncogene (derived from | 8.298E−03 | 4.59 | 71.75 |
| 435260 | H64245 | Hs. 34458 | ESTs | 2.194E−03 | 4.59 | 36.75 |
| 425100 | AF051850 | Hs. 154567 | supervillin | 5.865E−03 | 4.58 | 38.5 |
| 447023 | AA356764 | Hs. 17109 | integral membrane protein 2A | 6.056E−05 | 4.57 | 92 |
| 408705 | AA312135 | Hs. 46967 | HSPCO34 protein | 1.044E−06 | 4.57 | 62.5 |
| 424626 | AA344308 | Hs. 128427 | Homo sapiens BAC clone RP11-335J18 from | 1.399E−05 | 4.57 | 61.25 |
| 413786 | AW613780 | Hs. 13500 | ESTs | 1.966E−07 | 4.57 | 60 |
| 432572 | AI660840 | Hs. 191202 | ESTs, Weakly similar to ALUE_HUMAN !!!! | 7.522E−04 | 4.55 | 80.75 |
| 446927 | AW503484 | Hs. 16533 | myosin phosphatase, target subunit 1 | 1.596E−02 | 4.55 | 42.25 |
| 417052 | NM_000712 | Hs. 81029 | biliverdin reductase A | 5.081E−06 | 4.55 | 110 |
| 437456 | AL047045 | Hs. 60293 | Homo sapiens clone 122482 unknown mRNA | 2.477E−04 | 4.54 | 74.25 |
| 411590 | T96183 | | gb: ye09f07.s1 Stratagene lung (937210) H | 3.777E−03 | 4.53 | 46.75 |
| 401091 | F07783 | | decay accelerating factor for complement | 9.445E−04 | 4.52 | 56 |
| 423250 | BE061916 | Hs. 125849 | chromosome 8 open reading frame 2 | 1.077E−02 | 4.52 | 49.75 |
| 429732 | U20158 | Hs. 2488 | lymphocyte cytosolic protein 2 (SH2 doma | 1.292E−02 | 4.52 | 36.25 |
| 409549 | AB029015 | Hs. 54886 | phospholipase C, epsilon 2 | 3.841E−04 | 4.52 | 45.75 |
| 409461 | AA382169 | Hs. 54483 | N-myc (and STAT) interactor | 1.490E−07 | 4.50 | 197 |
| 451253 | H48299 | Hs. 26126 | claudin 10 | 9.284E−05 | 4.50 | 92.75 |
| 443119 | AA312264 | Hs. 7980 | hypothetical protein MGC12966 | 2.021E−04 | 4.50 | 43 |
| 423954 | AW753164 | Hs. 288604 | KIAA1632 protein | 1.241E−03 | 4.48 | 36.5 |
| 433226 | AW503733 | Hs. 9414 | KIAA1488 protein | 4.251E−04 | 4.47 | 68.25 |
| 429276 | AF056085 | Hs. 198612 | G protein-coupled receptor 51 | 2.232E−04 | 4.47 | 64.75 |
| 419743 | AW408762 | Hs. 5957 | Homo sapiens clone 24416 mRNA sequence | 8.609E−03 | 4.47 | 43.75 |
| 432967 | AA572949 | Hs. 207566 | ESTs | 5.637E−04 | 4.47 | 41.75 |
| 417973 | NM_004490 | Hs. 83070 | growth factor receptor-bound protein 14 | 1.339E−02 | 4.47 | 40.75 |
| 417954 | AI633943 | Hs. 26613 | ESTs, Weakly similar to no similarities | 3.787E−02 | 4.47 | 39.25 |
| 421654 | AW163267 | Hs. 106469 | suppressor of var1 (S. cerevisiae) 3-like | 2.178E−02 | 4.47 | 39.25 |
| 445776 | NM_001310 | Hs. 13313 | cAMP responsive element binding protein- | 1.339E−02 | 4.47 | 35.5 |
| 430008 | AW085625 | Hs. 186838 | ESTs, Weakly similar to Z295_HUMAN ZINC | 3.203E−03 | 4.47 | 55.5 |
| 421215 | AI868634 | Hs. 246358 | ESTs, Weakly similar to T3225tt hypotheti | 1.662E−05 | 4.46 | 71.75 |
| 430293 | AI416988 | Hs. 238272 | inositol 1,4,5-triphosphate receptor, ty | 1.149E−04 | 4.45 | 104.5 |
| 428342 | AI739168 | | Homo sapiens cDNA FLJ13458 fis, clone PL | 1.107E−05 | 4.45 | 85.5 |
| 446839 | BE091926 | Hs. 16244 | mitotic spindle coiled-coil related prot | 2.947E−03 | 4.45 | 53 |
| 434941 | AW073202 | Hs. 334825 | Homo sapiens cDNA FLJ14752 fis, clone NT | 8.634E−04 | 4.45 | 50.75 |
| 421181 | NM_005574 | Hs. 184585 | LIM domain only 2 (rhombotin-like 1) | 1.845E−03 | 4.45 | 39.25 |
| 421508 | NM_004833 | Hs. 105115 | absent in melanoma 2 | 7.103E−05 | 4.43 | 51.5 |
| 441652 | BE467811 | Hs. 7471 | BBP-like protein 1 | 9.630E−03 | 4.43 | 43.75 |
| 429105 | D87077 | Hs. 196275 | KIAA0240 protein | 2.412E−02 | 4.42 | 34.5 |
| 439223 | AW238299 | Hs. 250618 | UL16 binding protein 2 | 1.083E−03 | 4.42 | 41 |
| 446770 | AV660309 | Hs. 154986 | ESTs, Weakly similar to PLLP_HUMAN PLASM | 7.916E−02 | 4.42 | 45.25 |
| 436535 | AW295687 | Hs. 254420 | ESTs | 1.169E−05 | 4.40 | 59.5 |
| 413243 | AA769266 | Hs. 193657 | ESTs | 2.945E−03 | 4.40 | 52.25 |
| 408461 | AB037756 | Hs. 45207 | hypothetical protein KIAA1335 | 6.091E−03 | 4.40 | 36.75 |
| 432873 | AW837268 | Hs. 279639 | Homo sapiens mRNA; cDNA DKFZp586M2022 (f | 1.928E−03 | 4.40 | 34.25 |
| 416701 | R94977 | Hs. 35416 | PRO0132 protein | 1.928E−03 | 4.39 | 51.75 |
| 450746 | D82673 | Hs. 278589 | general transcription factor II. i | 7.536E−07 | 4.39 | 133.75 |
| 432435 | BE218886 | Hs. 282070 | ESTs | 9.808E−05 | 4.38 | 90.75 |
| 421685 | AF189723 | Hs. 106778 | ATPase, Ca transporting, type 2C, member | 5.412E−04 | 4.38 | 45.5 |
| 419951 | AI653415 | Hs. 195789 | ESTs | 9.022E−04 | 4.38 | 44.75 |
| 424960 | BE245380 | Hs. 153952 | 5′ nucleotidase (CD73) | 2.841E−02 | 4.38 | 40.25 |
| 410099 | AA081630 | | KIAA0036 gene product | 2.038E−02 | 4.38 | 35.25 |
| 422879 | AI241409 | Hs. 188092 | ESTs | 2.021E−04 | 4.35 | 65.75 |
| 438769 | AA830684 | Hs. 163426 | ESTs | 1.419E−03 | 4.35 | 64.75 |
| 443084 | AI827639 | Hs. 125539 | ESTs | 8.228E−04 | 4.35 | 62.75 |
| 417363 | AW129357 | Hs. 329700 | ESTs | 3.627E−03 | 4.35 | 51 |
| 408162 | AA993833 | Hs. 118527 | ESTs | 8.270E−03 | 4.35 | 33.5 |
| 421379 | Y15221 | Hs. 103982 | small inducible cytokine subfamily B (Cy | 3.401E−07 | 4.34 | 115.25 |
| 414462 | BE622743 | Hs. 301064 | arfaptin 1 | 1.299E−03 | 4.34 | 59.25 |
| 410245 | C17908 | Hs. 194125 | ESTs | 6.345E−03 | 4.32 | 44 |
| 434666 | AF151103 | Hs. 112259 | T cell receptor gamma locus | 1.146E−04 | 4.30 | 52.25 |
| 431328 | AA502999 | Hs. 291591 | ESTs | 1.211E−04 | 4.30 | 46 |
| 452939 | R35348 | Hs. 24970 | ESTs, Weakly similar to B34323 GTP-bindi | 5.213E−03 | 4.30 | 36.25 |
| 426030 | BE243933 | Hs. 108642 | zinc finger protein 22 (KOX 15) | 2.037E−02 | 4.30 | 38.75 |
| 417819 | AI253112 | Hs. 133540 | ESTs | 5.295E−02 | 4.30 | 36.25 |
| 429922 | Z97630 | Hs. 226117 | H1 histone family, member 0 | 2332E−02 | 4.29 | 48.5 |
| 428255 | AI627478 | Hs. 187670 | ESTs | 5.672E−04 | 4.28 | 44.25 |
| 407690 | R47799 | Hs. 266957 | hypothetical protein FLJ14281 | 2.107E−07 | 4.27 | 93.75 |
| 447887 | AA114050 | Hs. 19949 | caspase 8, apoptosis-related cysteine pr | 2.465E−04 | 4.27 | 50 |
| 446751 | AA766998 | Hs. 79126 | Human DNA sequence from clone RP11-16L21 | 6.861E−03 | 4.27 | 45.75 |
| 432610 | BE246615 | Hs. 278507 | histidyl-tRNA synthetase-like | 8.625E−04 | 4.27 | 38.5 |
| 434198 | AF119849 | Hs. 283028 | hypothetical protein PRO1598 | 1.417E−03 | 4.27 | 35.75 |
| 425387 | AB037864 | Hs. 156051 | KIAA1443 protein | 3.843E−04 | 4.27 | 35.75 |
| 407309 | AA526438 | Hs. 281680 | peroxisomal trans 2-enoyl CoA reductase; | 3.682E−05 | 4.27 | 41.75 |
| 429588 | AI080271 | Hs. 134533 | ESTs | 1.693E−03 | 4.27 | 48.25 |
| 434987 | AW975114 | Hs. 293273 | ESTs | 1.297E−03 | 4.25 | 79 |
| 432195 | AJ243669 | Hs. 8127 | KIAA0144 gene product | 7.510E−05 | 4.25 | 58 |
| 433847 | AA610266 | | ESTs | 2.477E−04 | 4.25 | 43 |
| 428720 | T90468 | Hs. 178154 | ESTs | 1.083E−03 | 4.25 | 39.5 |
| 419110 | AA234171 | Hs. 187626 | ESTs | 1.410E−04 | 4.25 | 35.75 |
| 411656 | AW855576 | | gb: CM4-CT0278-221099-027-d01 CT0278 Homo | 2.011E−03 | 4.25 | 32.5 |
| 448071 | BE621584 | Hs. 6983 | Homo sapiens cDNA: FLJ22646 fis, clone H | 6.035E−05 | 4.23 | 46 |
| 447513 | AW955776 | Hs. 313500 | ESTs, Moderately similar to ALU7_HUMAN A | 1.118E−06 | 4.22 | 84.75 |
| 440201 | AL359588 | Hs. 7041 | hypothetical protein DKFZp7628226 | 1.841E−03 | 4.22 | 42.75 |
| 425272 | AA354138 | Hs. 47209 | ESTs, Weakly similar to C35826 hypotheti | 2.599E−03 | 4.22 | 38.25 |
| 453793 | AK002178 | Hs. 35225 | hypothetical protein FLJ11316 | 7.404E−03 | 4.22 | 35 |
| 440193 | AW902312 | Hs. 7037 | Homo sapiens clone 24923 mRNA sequence | 3.909E−02 | 4.22 | 33.5 |
| 442061 | AA774284 | Hs. 285728 | abl-interactor 12 (SH3-containing protei | 8.228E−04 | 4.20 | 38.25 |
| 402507 | | | Target Exon | 5.392E−04 | 4.20 | 35.5 |
| 430569 | AF241254 | Hs. 178098 | angiotensin I converting enzyme (peptidy | 5.621E−02 | 4.20 | 35.5 |
| 454067 | AA041455 | Hs. 209312 | ESTs | 2.602E−04 | 4.18 | 44.5 |
| 431966 | AB037903 | Hs. 272257 | Homo sapiens truncated AKR mRNA for trun | 1.246E−02 | 4.18 | 40.5 |
| 452032 | BE244005 | Hs. 27610 | retinoic acid- and interferon inducible | 1.737E−04 | 4.17 | 70.25 |
| 402964 | | | NM_022095*: Homo sapiens hypothetical C2H | 1.134E−03 | 4.17 | 45.75 |
| 436860 | H12751 | Hs. 5327 | PRO1914protein | 7.165E−04 | 4.17 | 70.5 |
| 435513 | AW404075 | Hs. 42785 | DC11 protein | 1.156E−02 | 4.15 | 43.75 |
| 447094 | X65232 | Hs. 17364 | zinc finger protein 79 (pT7) | 4.674E−04 | 4.15 | 31.75 |
| 453394 | AW960474 | Hs. 40289 | ESTs | 1.483E−05 | 4.13 | 78.25 |
| 408392 | U28831 | Hs. 44566 | KIAA1641 protein | 8.228E−04 | 4.13 | 61.25 |
| 417601 | NM_014735 | Hs. 82292 | KIAA0215 gene product | 2.475E−04 | 4.13 | 45 |
| 406423 | | | C19000229*: gi\|6753826\|ref\|NP_034311.1\|f | 7.487E−02 | 4.13 | 31.25 |
| 435126 | AI393666 | Hs. 42315 | p10-binding protein | 1.967E−02 | 4.12 | 33.5 |
| 416987 | D86957 | Hs. 80712 | KIAA0202 protein | 1.298E−03 | 4.10 | 62.75 |
| 433556 | W56321 | Hs. 111460 | calcium/calmodulin-dependent protein kin | 1.084E−04 | 4.10 | 52.75 |
| 414449 | AA557660 | Hs. 76152 | decorin | 1.970E−02 | 4.10 | 46.5 |
| 420000 | AB036063 | Hs. 94262 | p53-inducible ribonucleotide reductase s | 5.963E−02 | 4.10 | 38.5 |
| 418720 | AI381687 | Hs. 39526 | ESTs | 1.038E−02 | 4.10 | 38 |
| 427205 | Z45791 | Hs. 173946 | hypothetical protein FLJ10486 | 2.178E−02 | 4.10 | 33 |
| 410541 | AA065003 | Hs. 64179 | syntenin-2 protein | 1.038E−03 | 4.09 | 82 |
| 411400 | AA311919 | Hs. 69851 | nucleolar protein family A, member 1 (H/ | 9.789E−05 | 4.08 | 54.75 |
| 422541 | NM_005131 | Hs. 1540 | nuclear matrix protein p84 | 2.284E−03 | 4.08 | 37.75 |
| 419943 | AA252111 | Hs. 15200 | ESTs | 2.874E−04 | 4.08 | 60.75 |
| 419195 | AW291165 | Hs. 25447 | ESTs | 1.135E−03 | 4.07 | 51.5 |
| 424939 | AK000059 | Hs. 153881 | Homo sapiens NY-REN-62 antigen mRNA, par | 5.299E−02 | 4.06 | 39.75 |
| 419590 | AF005043 | Hs. 91390 | poly (ADP-ribose) glycohydrolase | 9.272E−05 | 4.05 | 42 |
| 430468 | NM_004673 | Hs. 241519 | angiopoietin-like 1 | 3.204E−03 | 4.05 | 68.5 |
| 406038 | Y14443 | | zinc finger protein 200 | 1.546E−03 | 4.05 | 67.75 |
| 430522 | N75750 | Hs. 242271 | KIAA0471 gene product | 1.768E−03 | 4.05 | 41.25 |
| 424848 | AI263231 | Hs. 327090 | EST | 4.440E−03 | 4.05 | 61.75 |
| 452695 | AW780199 | Hs. 30327 | mitogen-activated protein kinase-activat | 1.480E−03 | 4.04 | 34.25 |
| 451593 | AF151879 | Hs. 26706 | CGI-121 protein | 8.336E−05 | 4.02 | 65.5 |
| 408548 | AA055449 | Hs. 63187 | ESTs, Weakly similar to ALUC_HUMAN !!!! | 1.620E−03 | 4.02 | 38.75 |
| 402439 | | | C1002445*: gi\|4506787\|ref\|NP_003861.1\|IQ | 3.342E−02 | 4.02 | 32.75 |
| 442202 | BE272862 | Hs. 106534 | hypothetical protein FLJ22625 | 1.565E−04 | 4.00 | 64.5 |
| 433233 | AB040927 | Hs. 301804 | KIAA1494 protein | 2.389E−03 | 4.00 | 43.5 |
| 407992 | AW418811 | | gb: ha21a06.x1 NCI_CGAP_Kid12 Homo sapien | 7.959E−03 | 4.00 | 38.25 |
| 450222 | U75308 | Hs. 24644 | TATA box binding protein (TBP)-associate | 5.152E−04 | 3.99 | 66.5 |
| 432676 | AI187366 | | gb: qf29c01.x1 Soares_testis_NHT Homo sap | 7.934E−05 | 3.98 | 49.25 |
| 427209 | H06509 | Hs. 92423 | KIAA1566 protein | 3.731E−06 | 3.98 | 126 |
| 416999 | AW195747 | Hs. 21122 | hypothetical protein FLJ11830 similar to | 1.088E−04 | 3.97 | 56.5 |
| 442485 | BE092285 | Hs. 29724 | hypothetical protein FLJ13187 | 4.100E−03 | 3.97 | 52.5 |
| 425395 | NM_014102 | Hs. 156243 | PRO1848 protein | 1.922E−04 | 3.97 | 44.75 |
| 424238 | AA337401 | Hs. 137635 | ESTs | 7.396E−03 | 3.97 | 36.75 |
| 400417 | X72475 | Hs. 156110 | Target | 1.903E−02 | 3.97 | 34.75 |
| 415323 | BE269352 | Hs. 949 | neutrophil cytosolic factor 2 (65 kD, chr | 7.689E−03 | 3.96 | 49.5 |
| 433017 | Y15067 | Hs. 279914 | zinc finger protein 232 | 3.665E−05 | 3.95 | 31 |
| 444985 | AI677737 | Hs. 28329 | hypothetical protein FLJ14005 | 7.496E−04 | 3.95 | 90.75 |
| 426108 | AA622037 | Hs. 166468 | programmed cell death 5 | 3.032E−02 | 3.95 | 43.5 |
| 441889 | AI090455 | Hs. 268371 | hypothetical protein FLJ20274 | 1.001E−02 | 3.95 | 38.5 |
| 443970 | AI280341 | Hs. 166571 | ESTs | 6.136E−02 | 3.95 | 37.75 |
| 449001 | AI619957 | | ESTs | 7.682E−03 | 3.93 | 33 |
| 427213 | AW007211 | | hypothetical protein FLJ 12876 | 2.601E−03 | 3.92 | 73.75 |
| 449964 | AW001741 | Hs. 24243 | hypothetical protein FLJ10706 | 3.843E−04 | 3.92 | 44.5 |
| 421443 | BE550141 | Hs. 156148 | hypothetical protein FLJ13231 | 3.019E−04 | 3.92 | 42.25 |
| 411412 | AJ001388 | Hs. 69997 | zinc finger protein 238 | 1.899E−02 | 3.92 | 32 |
| 449832 | AA694264 | Hs. 60049 | ESTs | 1.599E 02 | 3.92 | 38 |
| 419737 | H24185 | Hs. 92918 | hypothetical protein | 6.528E−04 | 3.90 | 45.5 |
| 436165 | AI373544 | Hs. 331328 | intermediate filament protein syncoilin | 7.106E−03 | 3.90 | 42.75 |
| 437862 | AW978107 | Hs. 5884 | Homo sapiens mRNA; cDNA DKFZp586C0224 (f | 1.187E−03 | 3.90 | 40.5 |
| 425210 | AA054679 | Hs. 155150 | ribonuclease P(14 kD) | 4.090E−03 | 3.90 | 31.25 |
| 442287 | AW952703 | Hs. 8182 | synaptic nuclei expressed gene 1b | 1.187E−03 | 3.88 | 55.5 |
| 439559 | AW364675 | Hs. 173921 | ESTs, Weakly similar to 2109260A B cell | 1.642E−04 | 3.88 | 58.25 |
| 450427 | AK001436 | Hs. 24994 | CGI-53 protein | 5.957E−02 | 3.88 | 32.25 |
| 421919 | AJ224901 | Hs. 109526 | zinc finger protein 198 | 1.925E−04 | 3.87 | 85.25 |
| 443601 | AI078554 | Hs. 15682 | ESTs | 3.122E−05 | 3.86 | 79.5 |
| 449509 | AA001615 | Hs. 84561 | ESTs | 7.111E−03 | 3.85 | 36.25 |
| 456107 | AA160000 | Hs. 137396 | ESTs, Weakly similar to JC5238 galactosy | 1.035E−02 | 3.85 | 30.25 |
| 422550 | BE297626 | Hs. 296049 | microfibrillar-associated protein 4 | 2.574E−02 | 3.85 | 51.25 |
| 408683 | R58665 | Hs. 46847 | TRAF and TNF receptor-associated protein | 4.448E−07 | 3.85 | 106 |
| 426860 | U04953 | Hs. 172801 | isoleucine-tRNA synthetase | 4.619E−03 | 3.84 | 45.5 |
| 438459 | T49300 | Hs. 35304 | Homo sapiens cDNA FLJ13655 fis. clone PL | 2.088E−05 | 3.83 | 80.75 |
| 431266 | AW149321 | Hs. 105411 | ESTs | 1.090E−04 | 3.83 | 99.75 |
| 418304 | AA215702 | | gb: zr97g10.r1 NCI_CGAP_GCB1 Homo sapiens | 7.169E−04 | 3.82 | 48 |
| 435354 | AA678267 | Hs. 117115 | ESTs | 1.968E−02 | 3.82 | 36.75 |
| 403575 | | | Target Exon | 6.831E−04 | 3.82 | 30.5 |
| 442961 | BE614474 | Hs. 289074 | F-box only protein 22 | 1.321E−05 | 3.82 | 76.25 |
| 412651 | AA115333 | Hs. 107968 | ESTs | 5.809E−07 | 3.81 | 128.75 |
| 448965 | AF092134 | Hs. 22679 | CGI-24 protein | 1.871E−05 | 3.81 | 60.5 |
| 450056 | BE047394 | Hs. 8208 | ESTs, Weakly similar to S71512 hypotheti | 7.505E−05 | 3.81 | 71 |
| 428330 | L22524 | Hs. 2256 | matrix metalloproleinase 7 (matrilysin, | 5.871E−03 | 3.81 | 77.25 |
| 428234 | U93553 | Hs. 183123 | nuclear receptor subfamily 5, group A, m | 5.142E−02 | 3.81 | 33 |
| 441646 | AB023169 | Hs. 7935 | KIAA0952 protein | 1.968E−02 | 3.80 | 38.25 |
| 402847 | | | C1000826*: gi\|12314084\|emb\|CAC05321.1\|(A | 1.156E−02 | 3.80 | 31.75 |
| 446207 | AW968535 | Hs. 14328 | hypothetical protein FLJ20071 | 1.489E−02 | 3.80 | 34.25 |
| 414658 | X58528 | Hs. 76781 | ATP-binding cassette, sub-family D (ALD) | 2.011E−03 | 3.80 | 61.5 |
| 421965 | AA301100 | Hs. 346482 | gb: EST14128 Testis tumor Homo sapiens cD | 1.663E−05 | 3.79 | 87.25 |
| 414219 | W20010 | Hs. 75823 | ALL1-fused gene from chromosome 1q | 2.180E−02 | 3.78 | 35.5 |
| 436385 | BE551618 | Hs. 144097 | ESTs | 7.990E−03 | 3.77 | 56 |
| 432689 | AB018320 | | Arg/Abl-interacting protein ArgBP2 | 6.099E−03 | 3.77 | 42.25 |
| 423450 | AJ290445 | Hs. 128759 | KIAA0524 protein | 2.602E−04 | 3.77 | 32.75 |
| 429031 | BE002237 | Hs. 239666 | Homo sapiens cDNA FLJ13495 fis, clone PL | 3.071E−03 | 3.77 | 31 |
| 434526 | AW085147 | Hs. 152779 | ESTs | 4.278E−02 | 3.77 | 30.75 |
| 416039 | AA376989 | Hs. 78989 | alcohol dehydrogenase 5 (class III), chi | 3.175E−04 | 3.77 | 72.75 |
| 414915 | NM_002462 | Hs. 76391 | myxovirus (influenza) resistance 1, homo | 7.398E−08 | 3.77 | 628.75 |
| 424003 | BE274717 | Hs. 137506 | Homo sapiens, clone IMAGE: 3605104, mRNA, | 5.008E−03 | 3.75 | 46 |
| 432409 | AA806538 | Hs. 130732 | KIAA1575 protein | 1.775E−02 | 3.75 | 36.75 |
| 415736 | AA827082 | Hs. 291872 | ESTs | 5.845E−03 | 3.75 | 36 |
| 423932 | T95633 | Hs. 189703 | ESTs | 7.687E−03 | 3.74 | 52.75 |
| 434733 | AI334367 | Hs. 159337 | ESTs | 1.341E−04 | 3.72 | 55.5 |
| 420926 | AA830402 | Hs. 221216 | ESTs | 2.037E−02 | 3.72 | 48 |
| 453070 | AK001465 | Hs. 31575 | SEC63, endoplasmic reticulum translocon | 1.339E−02 | 3.72 | 47.75 |
| 426310 | NM_000909 | Hs. 169266 | neuropeptide Y receptor Y1 | 7.692E−03 | 3.72 | 44.25 |
| 453753 | BE252983 | Hs. 35086 | ubiquitin specific protease 1 | 8.588E−03 | 3.72 | 41 |
| 430016 | NM_004736 | Hs. 227656 | xenotropic and polytropic retrovirus rec | 1.598E−02 | 3.72 | 37.5 |
| 401016 | | | ENSP00000227126: NAALADASE II PROTEIN. | 6.847E−03 | 3.72 | 35.5 |
| 447892 | AI435848 | Hs. 172978 | ESTs | 1.246E−02 | 3.72 | 34.25 |
| 425397 | J04088 | Hs. 156346 | topoisomerase (DNA) II alpha (170 kD) | 1.599E−02 | 3.72 | 31 |
| 407756 | AA116021 | Hs. 38260 | ubiquitin specific protease 18 | 1.600E−07 | 3.72 | 269.75 |
| 431392 | AI371223 | Hs. 288671 | Homo sapiens cDNA FLJ11997 fis, clone HE | 1.300E−03 | 3.72 | 60.5 |
| 451658 | AW195351 | Hs. 250520 | ESTs, Moderately similar to I38022 hypot | 6.060E−05 | 3.72 | 62.5 |
| 451406 | AI694320 | Hs. 6295 | ESTs, Weakly similar to T17248 hypotheti | 9.882E−04 | 3.71 | 56.25 |
| 408216 | AA741038 | Hs. 6670 | ESTs | 1.084E−03 | 3.70 | 58.75 |
| 417848 | AA206581 | Hs. 86041 | ESTs, Weakly similar to JC5314 CDC28/cdc | 7.162E−04 | 3.70 | 59.75 |
| 434924 | AA443164 | Hs. 23259 | hypothetical protein FLJ13433 | 1.321E−05 | 3.70 | 54 |
| 442085 | AA975688 | Hs. 159955 | ESTs | 3.339E−03 | 3.70 | 44.75 |
| 444363 | AI142827 | Hs. 143656 | ESTs | 2.723E−04 | 3.70 | 44 |
| 433891 | AA613792 | | gb: no97h03.s1 NCI_CGAP_Pr2 Homo sapiens | 4.811E−03 | 3.70 | 41 |
| 425423 | NM_005897 | Hs. 157180 | intracisternal A particle-promoted polyp | 1.915E−04 | 3.70 | 36.75 |
| 409493 | AA386192 | Hs. 193482 | Homo sapiens cDNA FLJ11903 fis, clone HE | 1.776E−02 | 3.70 | 34 |
| 451730 | AF095687 | Hs. 26937 | brain and nasopharyngeal carcinoma susce | 3.129E−02 | 3.70 | 34 |
| 419511 | AA429750 | Hs. 75113 | general transcription factor IIIA | 5.637E−03 | 3.70 | 30 |
| 452664 | AA398859 | Hs. 18397 | hypothetical protein FLJ23221 | 8.629E−07 | 3.69 | 124.5 |
| 430478 | NM_014349 | Hs. 241535 | apolipoprotein L, 3 | 5.537E−08 | 3.69 | 387 |
| 421406 | AF179897 | Hs. 104105 | Meis (mouse) homolog 2 | 5.643E−03 | 3.68 | 51.5 |
| 429686 | AI871613 | Hs. 28538 | Homo sapiens cDNA: FLJ21086 fis, clone C | 2.193E−03 | 3.67 | 48.75 |
| 429680 | AL035754 | Hs. 2474 | toll-like receptor 1 | 6.219E−04 | 3.67 | 38.25 |
| 423494 | AW504365 | Hs. 24143 | Wiskott-Aldrich syndrome protein interac | 3.203E−03 | 3.67 | 35.75 |
| 444545 | AW995346 | Hs. 146910 | ESTs | 1.899E−02 | 3.67 | 30.75 |
| 433907 | AW296107 | Hs. 152686 | ESTs | 5.660E−04 | 3.67 | 63.5 |
| 437650 | AA814338 | Hs. 292297 | ESTs | 7.118E−03 | 3.67 | 38.75 |
| 452279 | AA286844 | Hs. 61260 | hypothetical protein FLJ13164 | 7.283E−02 | 3.65 | 36.5 |
| 444969 | AI203334 | Hs. 160628 | ESTs | 9.976E−03 | 3.65 | 37.75 |
| 431560 | BE244135 | Hs. 260238 | hypothetical protein FLJ 10842 | 2.942E−03 | 3.65 | 37.5 |
| 425757 | AA363171 | | gb: EST72986 Ovary II Homo sapiens cDNA 5 | 1.341E−04 | 3.65 | 33 |
| 433464 | N92481 | | gb: zb12g02.s1Soares_fetal_lung_NbHL19W | 8.945E−03 | 3.65 | 30.25 |
| 446111 | W56338 | Hs. 13880 | CGI-143 protein | 2.290E−03 | 3.65 | 35.75 |
| 446506 | AI123118 | Hs. 15159 | chemokine-like factor, alternatively spl | 3.386E−07 | 3.63 | 131 |
| 436503 | AJ277750 | Hs. 183924 | ubiquitin associated and SH3 domain cont | 4.097E−03 | 3.63 | 33.5 |
| 451938 | AI354355 | Hs. 16697 | down-regulator of transcription 1, TBP-b | 1.921E−04 | 3.63 | 69.75 |
| 421114 | AW975051 | Hs. 293156 | ESTs, Weakly similar to I78885 serine/th | 4.444E−03 | 3.63 | 68.25 |
| 449720 | AA311152 | Hs. 288708 | hypothetical protein FLJ21562 | 4.468E−06 | 3.63 | 63.75 |
| 443373 | AI792868 | Hs. 135365 | ESTs | 4.099E−03 | 3.63 | 49 |
| 417148 | AA359896 | Hs. 293885 | hypothetical protein FLJ14902 | 1.837E−02 | 3.63 | 49 |
| 457231 | AI472022 | Hs. 301959 | proline synthetase co-transcribed (bacte | 3.450E−02 | 3.63 | 32.75 |
| 408380 | AF123050 | Hs. 44532 | diubiquitin | 4.434E−07 | 3.62 | 217.25 |
| 425692 | D90041 | Hs. 155956 | N-acetyltransferase 1 (arylamine N-acety | 1.760E−05 | 3.60 | 66.5 |
| 443968 | AA287702 | Hs. 10031 | KIAA0955 protein | 8.945E−03 | 3.60 | 58.25 |
| 428250 | AW809208 | Hs. 183297 | DKFZP566F2124 protein | 5.932E−04 | 3.60 | 54 |
| 407946 | AA226495 | Hs. 154292 | ESTs | 1.903E−04 | 3.60 | 53 |
| 412828 | AL133396 | Hs. 74621 | prion protein (p27-30) (Creutzfeld-Jakob | 4.990E−02 | 3.60 | 50.25 |
| 418699 | BE539639 | Hs. 173030 | ESTs, Weakly similar to ALU8_HUMAN ALU S | 4.813E−03 | 3.60 | 43.5 |
| 431416 | AA532718 | Hs. 178604 | ESTs | 8.139E−02 | 3.60 | 37 |
| 425345 | AU077297 | Hs. 155894 | protein tyrosine phosphatase, non-recept | 3.773E−03 | 3.60 | 36.75 |
| 422303 | AW410382 | Hs. 27556 | hypothetical protein FLJ22405 | 5.301E−02 | 3.60 | 36.75 |
| 456760 | AW961251 | Hs. 127828 | guanine nucleotide binding protein (G pr | 1.084E−03 | 3.60 | 33 |
| 434936 | AI285970 | Hs. 183817 | ESTs | 6.881E−02 | 3.59 | 56.25 |
| 448192 | R43915 | Hs. 4958 | ESTs | 4.440E−03 | 3.58 | 31 |
| 408618 | AK000637 | Hs. 46624 | HSPC043 protein | 7.694E−03 | 3.58 | 43.25 |
| 427051 | BE178110 | Hs. 173374 | Homo sapiens cDNA FLJ10500 fis, clone NT | 1.201E−02 | 3.58 | 40 |
| 430024 | AI808780 | Hs. 227730 | integrin, alpha 6 | 7.913E−05 | 3.57 | 68.75 |
| 424881 | AL119690 | Hs. 153618 | HCGVIII-1 protein | 2.010E−03 | 3.57 | 40.25 |
| 408438 | AB011180 | Hs. 100960 | KIAA0608 protein | 1.076E−02 | 3.57 | 38 |
| 427471 | AA403131 | Hs. 266782 | KIAA1826 protein | 2.494E−03 | 3.57 | 34 |
| 432769 | AA620814 | Hs. 144959 | ESTs | 4.558E−02 | 3.57 | 31.25 |
| 426181 | AA371422 | Hs. 334371 | hypothetical protein MGC13096 | 7.162E−04 | 3.57 | 34.75 |
| 439195 | H89360 | | gb: yw28d08.s1 Morton Fetal Cochlea Homo | 4.621E−03 | 3.57 | 41.75 |
| 444020 | R92962 | Hs. 35052 | ESTs | 1.970E−02 | 3.57 | 48.75 |
| 419908 | AW971327 | Hs. 293315 | ESTs | 9.289E−03 | 3.56 | 32 |
| 427581 | NM_014788 | Hs. 179703 | KIAA0129 gene product | 1.489E−07 | 3.55 | 117.5 |
| 419440 | AB020689 | Hs. 90419 | KIAA0882 protein | 2.478E−04 | 3.55 | 57.75 |
| 409342 | AU077058 | Hs. 54089 | BRCA1 associated RING domain 1 | 6.528E−04 | 3.55 | 47.25 |
| 410054 | AL120050 | Hs. 58220 | Homo sapiens cDNA: FLJ23005 fis, clone L | 1.076E−02 | 3.55 | 38 |
| 437133 | AB018319 | Hs. 5460 | KIAA0776 protein | 1.774E−02 | 3.55 | 35.25 |
| 423886 | AA332098 | | gb: EST36256 Embryo, 8 week I Homo sapien | 1.116E−02 | 3.55 | 33 |
| 432540 | AI821517 | Hs. 105866 | ESTs | 3.620E−03 | 3.54 | 43.25 |
| 402737 | | | Target Exon | 4.105E−05 | 3.54 | 38.75 |
| 421977 | W94197 | Hs. 110165 | ribosomal protein L26 homolog | 6.855E−02 | 3.54 | 40 |
| 418222 | AI675881 | Hs. 86538 | ESTs | 2.592E−03 | 3.53 | 40.5 |
| 418793 | AW382987 | Hs. 88474 | prostaglandin-endoperoxide synthase 1 (p | 8.283E−03 | 3.53 | 34.75 |
| 417301 | AI478158 | Hs. 164478 | hypothetical protein FLJ21939 similar to | 1.083E−03 | 3.52 | 51 |
| 412863 | AA121673 | Hs. 59757 | zinc finger protein 281 | 2.250E−02 | 3.52 | 45.25 |
| 439763 | AA845366 | Hs. 184075 | ESTs, Weakly similar to ALU1_HUMAN ALU S | 1.033E−03 | 3.52 | 42.25 |
| 403809 | | | NM_024743*: Homo sapiens hypothetical pro | 1.490E−02 | 3.52 | 41.75 |
| 421684 | BE281591 | Hs. 106768 | hypothetical protein FLJ10511 | 1.117E−02 | 3.52 | 41.75 |
| 436271 | AW449686 | Hs. 129828 | ESTs | 1.563E−04 | 3.52 | 37.25 |
| 445240 | AI217385 | Hs. 147574 | ESTs | 1.599E−02 | 3.52 | 34.5 |
| 432834 | F06459 | Hs. 289113 | cytochrome b5 reductase 1 (B5R.1) | 9.286E−03 | 3.52 | 31.75 |
| 426780 | BE242284 | Hs. 172199 | adenylate cyclase 7 | 2.871E−04 | 3.50 | 55.75 |
| 430750 | AI650360 | Hs. 100256 | ESTs | 1.615E−03 | 3.50 | 39.5 |
| 429301 | AA449416 | Hs. 31395 | ESTs | 6.828E−04 | 3.50 | 35 |
| 418196 | AI745649 | Hs. 26549 | KIAA1708 protein | 4.422E−02 | 3.50 | 34.5 |
| 421951 | BE327432 | Hs. 109804 | H1 histone family, member X | 2.485E−03 | 3.50 | 33.5 |
| 419825 | AI754011 | Hs. 7326 | ESTs | 6.101E−03 | 3.50 | 31.25 |
| 443303 | U67319 | Hs. 9216 | caspase 7, apoptosis-related cysteine pr | 4.604E−05 | 3.49 | 92.25 |
| 451119 | AA805417 | Hs. 64753 | ESTs | 4.088E−03 | 3.49 | 34.25 |
| 431315 | AW972227 | Hs. 163986 | Homo sapiens cDNA: FLJ22765 fis, clone K | 2.951E−05 | 3.49 | 159.25 |
| 408731 | R85652 | | Homo sapiens mRNA; cDNA DKFZp434F1928 (f | 3.690E−05 | 3.49 | 75.25 |
| 422423 | AF283777 | Hs. 116481 | CD72 antigen | 1.000E−02 | 3.48 | 39 |
| 437664 | AW977714 | Hs. 294139 | ESTs, Moderately similar to ALU1_HUMAN A | 2.497E−03 | 3.47 | 74.25 |
| 448219 | AA228092 | | KIAA1681 protein | 9.459E−04 | 3.47 | 56.5 |
| 408784 | AW971350 | Hs. 63386 | ESTs | 2.938E−05 | 3.47 | 46 |
| 417562 | AW888754 | Hs. 134126 | crystallin, gamma S | 1.926E−03 | 3.47 | 33.5 |
| 414821 | M63835 | Hs. 77424 | Fc fragment of IgG, high affinity la, re | 1.246E−02 | 3.47 | 36.5 |
| 408088 | AW157022 | Hs. 343551 | hypothetical protein FLJ 22584 | 4.114E−05 | 3.46 | 32 |
| 417008 | AA191708 | Hs. 325825 | Homo sapiens cDNA FLJ20848 fis, clone AD | 2.243E−04 | 3.46 | 64.5 |
| 446238 | T95143 | Hs. 14511 | SCO (cytochrome oxidase deficient, yeast | 7.107E−05 | 3.45 | 87 |
| 446162 | AI631319 | Hs. 63841 | hypothetical protein DKFZp434E2318 | 2.013E−03 | 3.45 | 41.25 |
| 453686 | AL110326 | Hs. 304679 | ESTs, Moderately similar to Z195_HUMAN Z | 1.270E−04 | 3.45 | 41.25 |
| 443242 | BE243910 | Hs. 9082 | nucleoporin p54 | 6.587E−03 | 3.45 | 36 |
| 409939 | AA463437 | Hs. 11556 | Homo sapiens cDNA FLJ12566 fis, clone NT | 6.384E−05 | 3.45 | 71 |
| 418584 | NM_004606 | Hs. 1179 | TATA box binding protein (TBP)-associate | 1.241E−03 | 3.44 | 37.25 |
| 413129 | AF292100 | Hs. 104613 | RP42 homolog | 1.033E−04 | 3.44 | 106.75 |
| 416980 | AA381133 | Hs. 80684 | high-mobility group (nonhistone chromoso | 4.270E−03 | 3.42 | 67.75 |
| 450850 | AA648886 | Hs. 151999 | ESTs | 8.248E−04 | 3.42 | 55.25 |
| 426494 | AL119528 | Hs. 170098 | KIAA0372 gene product | 6.836E−03 | 3.42 | 45.5 |
| 417244 | T57053 | Hs. 10136 | ESTs | 2.711E−03 | 3.42 | 33.5 |
| 413392 | AW021404 | Hs. 13021 | ESTs | 2.827E−03 | 3.42 | 33 |
| 427722 | AK000123 | Hs. 180479 | hypothetical protein FLJ20116 | 3.233E−02 | 3.42 | 32.75 |
| 441466 | AW673081 | Hs. 54828 | ESTs | 5.005E−03 | 3.42 | 32 |
| 412675 | AA460716 | Hs. 9788 | hypothetical protein MGC10924 similar to | 1.489E−02 | 3.41 | 415 |
| 439653 | AW021103 | Hs. 6631 | hypothetical protein FLJ20373 | 2.954E−03 | 3.40 | 46.25 |
| 418259 | AA215404 | | ESTs | 2.628E−05 | 3.40 | 84 |
| 423032 | AI684746 | Hs. 119274 | RAS p21 protein activator (GTPase activa | 1.413E−04 | 3.40 | 71 |
| 452167 | N75238 | Hs. 13075 | Homo sapiens cDNA: FLJ23013 fis, clone L | 4.444E−03 | 3.40 | 37 |
| 408989 | AW361666 | Hs. 49500 | KIAA0746 protein | 1.662E−05 | 3.40 | 99.5 |
| 446934 | AK001943 | Hs. 16577 | F-box only protein 3 | 1.768E−03 | 3.38 | 64.25 |
| 420664 | AI681270 | Hs. 99824 | BCE-1 protein | 7.518E−04 | 3.38 | 61.5 |
| 444430 | AI611153 | Hs. 6093 | Homo sapiens cDNA: FLJ22783 fis, clone K | 1.246E−02 | 3.38 | 45.25 |
| 437410 | AW023340 | Hs. 14880 | ESTs | 7.959E−03 | 3.38 | 43.25 |
| 426979 | AF161472 | Hs. 173074 | DKFZP564O1863 protein | 7.985E−03 | 3.38 | 40.75 |
| 422316 | N75612 | Hs. 301497 | arginyltransferase 1 | 5.639E−03 | 3.38 | 35.75 |
| 421743 | T35958 | Hs. 107614 | DKFZP564I1171 protein | 2.346E−05 | 3.36 | 40.75 |
| 418721 | NM_002731 | Hs. 87773 | protein kinase, cAMP-dependent, catalyti | 3.687E−05 | 3.36 | 62 |
| 431742 | NM_016652 | Hs. 268281 | crooked neck protein (crn) | 4.093E−03 | 3.36 | 42.5 |
| 422473 | U94780 | Hs. 117242 | meningioma expressed antigen 6 (coiled-c | 1.417E−03 | 3.36 | 39.5 |
| 452194 | AI694413 | Hs. 332649 | olfactory receptor, family 2, subfamily | 6.214E−07 | 3.36 | 357.25 |
| 436372 | AW972301 | Hs. 310286 | ESTs | 8.217E−06 | 3.36 | 150.25 |
| 444454 | BE018316 | Hs. 11183 | sorting nexin 2 | 3.489E−05 | 3.35 | 52.75 |
| 439717 | W94472 | Hs. 59529 | ESTs, Moderately similar to ALU1_HUMAN A | 5.865E−03 | 3.35 | 42.25 |
| 412634 | U55984 | Hs. 289088 | heat shock 90 kD protein 1, alpha | 5.358E−06 | 3.35 | 39 |
| 419970 | AW612022 | Hs. 94812 | ESTs | 3.201E−03 | 3.35 | 35.75 |
| 447514 | AI809314 | Hs. 208501 | ESTs, Weakly similar to B34087 hypotheti | 4.558E−02 | 3.35 | 32 |
| 444013 | T08531 | Hs. 44404 | Homo sapiens PRO1488 mRNA, complete cds | 7.116E−03 | 3.35 | 31.75 |
| 445718 | H79791 | Hs. 15227 | ESTs | 9.273E−06 | 3.35 | 156.75 |
| 437025 | AW296618 | Hs. 120637 | ESTs | 5.623E−02 | 3.35 | 30.5 |
| 425462 | AI491852 | Hs. 46783 | Homo sapiens cDNA: FLJ22382 fis, clone H | 1.657E−02 | 3.34 | 31 |
| 440726 | AL050333 | Hs. 7387 | DKFZP564B116 protein | 3.209E−03 | 3.34 | 74.25 |
| 417377 | NM_016603 | Hs. 82035 | potential nuclear protein C5ORF5; GAP li | 3.731E−06 | 3.34 | 66 |
| 452548 | AL050321 | Hs. 301532 | CRP2 binding protein | 1.919E−04 | 3.33 | 54.25 |
| 442853 | AW021276 | Hs. 17121 | ESTs | 7.401E−03 | 3.33 | 31.5 |
| 444745 | AF117754 | Hs. 11861 | thyroid hormone receptor-associated prot | 2.599E−04 | 3.33 | 90.75 |
| 438543 | AA810141 | Hs. 192182 | ESTs | 8.798E−05 | 3.32 | 59.75 |
| 447188 | H65423 | Hs. 17631 | hypothetical protein DKFZp434E2135 | 5.209E−03 | 3.32 | 59.25 |
| 454064 | AI130731 | Hs. 57967 | ESTs | 7.687E−03 | 3.32 | 32.25 |
| 433269 | AI343543 | Hs. 126890 | ESTs | 3.168E−04 | 3.32 | 31.75 |
| 407813 | AL120247 | Hs. 40109 | KIAA0872 protein | 1.105E−05 | 3.32 | 66.25 |
| 452696 | AI826645 | Hs. 211534 | ESTs | 6.450E−06 | 3.32 | 55.75 |
| 430007 | NM_014892 | Hs. 227602 | KIAA1116 protein | 5.941E−04 | 3.32 | 59.75 |
| 457247 | AA458605 | | KIAA1681 protein | 7.702E−02 | 3.30 | 32.25 |
| 431451 | AA761378 | Hs. 192013 | ESTs | 5.298E−02 | 3.30 | 39.5 |
| 442160 | AI337127 | Hs. 156325 | ESTs | 7.713E−06 | 3.30 | 67.75 |
| 420962 | NM_005904 | Hs. 100602 | MAD (mothers against decapentaplegic, Dr | 9.242E−06 | 3.30 | 126.25 |
| 419284 | AW820869 | Hs. 215658 | ESTs, Moderately similar to ZN91_HUMAN Z | 5.450E−02 | 3.29 | 39.5 |
| 441297 | AW403084 | Hs. 7766 | ubiquitin-conjugating enzyme E2E 1 (homo | 7.876E−04 | 3.29 | 52 |
| 444030 | AW021254 | Hs. 135055 | ESTs | 2.194E−03 | 3.27 | 53.5 |
| 427675 | AW138190 | Hs. 180248 | zinc finger protein 124 (HZF-16) | 5.121E−05 | 3.27 | 45 |
| 408558 | AW015759 | Hs. 235709 | Homo sapiens mRNA; cDNA DKFZp667B0711 (f | 4.420E−02 | 3.27 | 37.5 |
| 418945 | BE246762 | Hs. 89499 | arachidonale 5-lipoxygenase | 1.143E−04 | 3.27 | 37 |
| 445733 | BE295568 | Hs. 13225 | UDP-Gal: betaGlcNAc beta 1,4-galactosylt | 6.592E−03 | 3.27 | 37.5 |
| 436139 | AA765786 | Hs. 120936 | ESTs | 1.033E−04 | 3.26 | 62.25 |
| 443405 | AF031463 | Hs. 9302 | phosducin-like | 1.145E−04 | 3.26 | 53.75 |
| 432841 | M93425 | Hs. 62 | protein tyrosine phosphatase, non-recept | 3.344E−03 | 3.26 | 48.5 |
| 449188 | AW072939 | Hs. 347187 | myotubularin related protein 1 | 1.757E−05 | 3.25 | 59.75 |
| 441077 | AI241273 | H5.15312 | ESTs | 9.823E−07 | 3.25 | 76 |
| 442297 | NM_006202 | Hs. 89901 | phosphodiesterase 4A, cAMP-specific (dun | 1.296E−03 | 3.25 | 36.5 |
| 449439 | AB029001 | Hs. 23585 | KIAA1078 protein | 2.409E−02 | 3.25 | 33 |
| 417665 | AW852858 | Hs. 22862 | ESTs | 1.001E−02 | 3.25 | 32.75 |
| 420174 | AI824144 | Hs. 23912 | ESTs | 2.221E−05 | 3.25 | 47.75 |
| 430929 | AA489166 | Hs. 156933 | ESTs | 3.501E−04 | 3.22 | 55.5 |
| 453128 | AW026516 | Hs. 31791 | acylphosphatase 2, muscle type | 3.777E−03 | 3.22 | 46 |
| 422932 | AI191813 | Hs. 308220 | ESTs | 1.480E−03 | 3.22 | 35.25 |
| 428004 | AA449563 | Hs. 151393 | glutamate-cysteine ligase, catalytic sub | 4.844E−02 | 3.21 | 38.75 |
| 426221 | AB007881 | Hs. 110613 | KIAA0421 protein | 6.525E−04 | 3.21 | 58.5 |
| 426312 | AF026939 | Hs. 181874 | interferon-induced protein with tetratri | 1.127E−07 | 3.20 | 172.5 |
| 417678 | X06560 | Hs. 82396 | 2′,5′-oligoadenylate synthetase 1 (40-46 | 1.973E−07 | 3.20 | 195.5 |
| 436854 | AA749167 | Hs. 173911 | ESTs | 2.285E−03 | 3.20 | 42 |
| 418180 | BE618087 | Hs. 83724 | hypothetical protein MGC5466 | 5.144E−02 | 3.19 | 31.25 |
| 414895 | AW894856 | Hs. 116278 | Homo sapiens cDNA FLJ13571 fis, clone PL | 9.877E−04 | 3.19 | 46.5 |
| 407765 | AW076027 | Hs. 257711 | ESTs, Moderately similar to ALU8_HUMAN A | 5.639E−03 | 3.18 | 57.75 |
| 448914 | AI927656 | Hs. 196459 | ESTs | 1.966E−02 | 3.17 | 45.25 |
| 447371 | AA334274 | Hs. 18368 | DKFZP564B0769 protein | 4.444E−03 | 3.17 | 41.75 |
| 439680 | AW245741 | Hs. 58461 | ESTs, Weakly similar to A35659 krueppel- | 1.036E−03 | 3.17 | 33.25 |
| 431188 | W05656 | Hs. 169755 | ESTs | 5.217E−03 | 3.17 | 32.5 |
| 446177 | AK001902 | Hs. 14202 | hypothetical protein FLJ11040 | 6.019E−05 | 3.17 | 56.5 |
| 415914 | AA306033 | Hs. 78915 | GA-binding protein transcription factor, | 3.910E−02 | 3.17 | 38 |
| 408761 | AA057264 | Hs. 238936 | ESTs, Weakly similar to (defline not ava | 2.233E−04 | 3.17 | 48.25 |
| 408050 | BE280478 | Hs. 182695 | hypothetical protein MGC3243 | 3.934E−03 | 3.16 | 33 |
| 444342 | NM_014398 | Hs. 10887 | similar to lysosome-associated membrane | 1.048E−07 | 3.15 | 116.75 |
| 441879 | AI521936 | Hs. 107149 | novel protein similar to archaeal, yeast | 2.630E−05 | 3.15 | 72.25 |
| 436169 | AA888311 | Hs. 17602 | Homo sapiens cDNA FLJ12381 fis, clone MA | 5.121E−05 | 3.15 | 68.5 |
| 426506 | AW935187 | Hs. 170162 | KIAA1357 protein | 5.124E−05 | 3.15 | 66.25 |
| 410390 | AA876905 | Hs. 125286 | ESTs | 4.447E−03 | 3.15 | 63.5 |
| 410614 | AI091195 | Hs. 65029 | growth arrest-specific 1 | 3.559E−02 | 3.15 | 43.25 |
| 407821 | AA346172 | Hs. 195614 | ESTs | 7.489E−04 | 3.15 | 35 |
| 433364 | AI075407 | Hs. 296083 | ESTs, Moderately similar to I54374 gene | 5.558E−08 | 3.15 | 474.5 |
| 408145 | AF182316 | Hs. 234680 | fer-1 (C. elegans)-like 3 (myoferlin) | 6.880E−02 | 3.14 | 49.25 |
| 411060 | NM_006074 | Hs. 318501 | Homo sapiens mRNA full length insert cDN | 9.799E−08 | 3.14 | 238.75 |
| 421272 | AA704157 | | ESTs | 1.921E−04 | 3.14 | 46 |
| 442739 | NM_007274 | Hs. 8679 | cytosolic acyl coenzyme A thioester hydr | 6.515E−04 | 3.14 | 42.25 |
| 438021 | AV653790 | Hs. 324275 | WW domain-containing protein 1 | 7.183E−04 | 3.13 | 120.25 |
| 418027 | AB037807 | Hs. 83293 | hypothetical protein | 2.632E−05 | 3.13 | 71.5 |
| 443015 | R33261 | Hs. 6614 | ESTs, Weakly similar to A43932 mucin 2 p | 1.540E−02 | 3.13 | 36.75 |
| 450374 | AA397540 | Hs. 60293 | Homo sapiens clone 122482 unknown mRNA | 2.842E−02 | 3.13 | 32.75 |
| 430068 | AA464964 | | gb: zx80f10.s1 Soares ovary tumor NbHOT H | 4.601E−05 | 3.12 | 47.75 |
| 452699 | AW295390 | Hs. 213062 | ESTs | 1.843E−03 | 3.11 | 52.25 |
| 453822 | NM_014116 | Hs. 35416 | PRO0132 protein | 7.490E−02 | 3.11 | 39 |
| 426647 | AA243464 | Hs. 294101 | pre-B-cell leukemia transcription factor | 1.084E−03 | 3.10 | 52 |
| 434629 | AA789081 | Hs. 4029 | glioma-amplified sequence-41 | 1.135E−03 | 3.10 | 50.75 |
| 432680 | T47364 | Hs. 278613 | interferon, alpha-inducible protein 27 | 5.551E−08 | 3.10 | 1337.75 |
| 403738 | | | C4000675*: gi\|3426332\|gb\|AAC32272.1\|(AF0 | 1.571E−05 | 3.10 | 108 |
| 418627 | AL079835 | Hs. 86858 | ribosomal protein S6 kinase, 70 kD, polyp | 5.652E−04 | 3.09 | 35.5 |
| 457701 | AW855466 | Hs. 271866 | ESTs, Weakly similar to ALU1_HUMAN ALU S | 5.944E−04 | 3.08 | 55.75 |
| 458368 | BE504731 | Hs. 138827 | ESTs | 8.248E−04 | 3.08 | 64.5 |
| 449656 | AA002008 | Hs. 188633 | ESTs | 1.552E−03 | 3.07 | 72.5 |
| 448362 | AA641767 | Hs. 21015 | hypothetical protein DKFZp564L0864 simil | 1.117E−02 | 3.07 | 43.5 |
| 425815 | R94023 | Hs. 94560 | ESTs, Moderately similar to I38022 hypot | 2.127E−04 | 3.07 | 38.5 |
| 410851 | AW612147 | Hs. 32058 | Homo sapiens C1orf19 mRNA, partial cds | 4.627E−03 | 3.07 | 35.5 |
| 420258 | AA477514 | Hs. 96247 | translin-associated factor X | 1.387E−02 | 3.07 | 30.5 |
| 452253 | AA928891 | Hs. 28608 | Homo sapiens cDNA: FLJ22115 fis, clone H | 2.231E−04 | 3.07 | 42.5 |
| 417317 | AW296584 | Hs. 293782 | ESTs | 4.476E−06 | 3.07 | 78 |
| 408212 | AA297567 | Hs. 43728 | hypothetical protein | 3.326E−04 | 3.07 | 47 |
| 413278 | BE563085 | Hs. 833 | interferon-stimulated protein, 15 kDa | 5.558E−08 | 3.06 | 867.25 |
| 419257 | X53461 | Hs. 89781 | upstream binding transcription factor, R | 3.687E−05 | 3.05 | 59 |
| 424637 | NM_015057 | Hs. 151411 | KIAA0916 protein | 1.355E−06 | 3.05 | 82 |
| 414948 | C15240 | Hs. 182155 | ESTs | 2.485E−05 | 3.05 | 45.5 |
| 418677 | S83308 | Hs. 87224 | SRY (sex determining region Y)-box 5 | 1.000E−02 | 3.05 | 41.25 |
| 419465 | AW500239 | Hs. 21187 | Homo sapiens cDNA: FLJ23068 fis, clone L | 1.338E−02 | 3.05 | 35.75 |
| 443787 | AV646505 | Hs. 122155 | ESTs | 2.599E−03 | 3.05 | 31.25 |
| 438980 | AW502384 | | gb: UI-HF-BR0p-aka-f-12-0-UI.r1NIH_MGC_5 | 2.102E−03 | 3.03 | 59 |
| 440668 | AI989538 | Hs. 191074 | ESTs | 1.187E−03 | 3.03 | 55.25 |
| 407687 | AK002011 | Hs. 37558 | hypothetical protein FLJ11149 | 7.848E−04 | 3.03 | 37.5 |
| 409977 | AW805510 | Hs. 97056 | hypothetical protein FLJ21634 | 2.218E−05 | 3.02 | 74 |
| 448030 | N30714 | Hs. 325960 | membrane-spanning 4-domains, subfamily A | 2.032E−02 | 3.02 | 50.5 |
| 407705 | AB023139 | Hs. 37892 | KIAA0922 protein | 1.408E−04 | 3.02 | 49.75 |
| 450205 | AI219748 | Hs. 11356 | ESTs | 6.495E−02 | 3.02 | 47.75 |
| 437086 | AW291411 | Hs. 192531 | ESTs, Weakly similar to S00754 zinc fing | 1.827E−04 | 3.02 | 46 |
| 411960 | R77776 | Hs. 18103 | ESTs | 1.416E−03 | 3.02 | 37.25 |
| 418757 | AI864193 | Hs. 169728 | hypothetical protein FLJ13150 | 6.852E−04 | 3.02 | 41.5 |
| 434045 | AI065133 | Hs. 152316 | hypothetical protein PRO0971 | 4.461E−04 | 3.02 | 46.5 |
| 414617 | AI339520 | Hs. 288817 | ESTs, Moderately similar to N Chain N, M | 1.487E−05 | 3.02 | 183.25 |
| 431709 | AF220185 | Hs. 267923 | uncharacterized hypothalamus protein HT0 | 2.596E−04 | 3.01 | 42.75 |
| 431341 | AA307211 | Hs. 251531 | proteasome (prosome, macropain) subunit, | 2.126E−04 | 3.01 | 60.25 |
| 409884 | AI904455 | Hs. 142684 | hypothetical protein DKFZp667O116 | 3.020E−04 | 3.00 | 68 |
| 422231 | AA443512 | Hs. 101383 | ESTs | 4.601E−05 | 3.00 | 65 |
| 418460 | M26315 | Hs. 85258 | CD8 antigen, alpha polypeptide (p32) | 1.147E−04 | 3.00 | 63 |
| 446851 | AW007332 | Hs. 10450 | Homo sapiens cDNA: FLJ22063 fis, clone H | 8.341E−05 | 3.00 | 49 |
| 411127 | AA668995 | Hs. 218329 | hypothetical protein | 4.090E−03 | 3.00 | 35.5 |
| 432474 | AA584042 | | gb: nn65e09.s1 NCI_CGAP_Lar1 Homo sapiens | 1.596E−02 | 3.00 | 34.75 |
| 451171 | AA248829 | Hs. 112921 | gb: jj6059.seq.F Human fetal heart, Lambd | 9.877E−04 | 3.00 | 33.5 |
| 418182 | AW016405 | Hs. 16648 | ESTs | 1.239E−03 | 3.00 | 33 |
| 414033 | AL079707 | Hs. 207443 | hypothetical protein MGC10848 | 1.930E−03 | 3.00 | 31.5 |
| 431542 | H63010 | Hs. 5740 | ESTs | 1.117E−02 | 3.00 | 31 |
| 414493 | AL133921 | Hs. 76272 | retinoblastoma-binding protein 2 | 1.662E−05 | 2.99 | 90.25 |
| 428418 | AI368826 | Hs. 30654 | ESTs | 1.321E−05 | 2.99 | 85.75 |
| 431629 | AU077025 | Hs. 265827 | interferon, alpha-inducible protein (clo | 8.507E−08 | 2.99 | 674.75 |
| 439658 | AA332057 | Hs. 6639 | hypothetical protein MGC15440 | 7.495E−05 | 2.99 | 47.75 |
| 425332 | AA633306 | Hs. 127279 | ESTs | 6.333E−03 | 2.99 | 40.25 |
| 410678 | BE540516 | Hs. 293732 | hypothetical protein MGC3195 | 7.522E−04 | 2.98 | 54 |
| 452420 | BE564871 | Hs. 29463 | centrin, EF-hand protein, 3 (CDC31 yeast | 4.480E−06 | 2.97 | 69.75 |
| 451572 | AA018556 | Hs. 268691 | ESTs, Moderately similar to ALU2_HUMAN A | 2.219E−05 | 2.97 | 52.5 |
| 411190 | AA306342 | Hs. 69171 | protein kinase C-like 2 | 8.296E−03 | 2.97 | 46 |
| 444437 | AI377961 | Hs. 44041 | ESTs | 6.592E−03 | 2.97 | 34 |
| 425548 | AA890023 | Hs. 1906 | prolactin receptor | 5.657E−04 | 2.97 | 39 |
| 413568 | AA130381 | Hs. 180257 | ESTs | 2.938E−05 | 2.97 | 45.25 |
| 419925 | AA159850 | Hs. 93765 | lipoma HMGIC fusion partner | 3.790E−02 | 2.97 | 42.75 |
| 446215 | AW821329 | Hs. 14368 | SH3 domain binding glutamic acid-rich pr | 2.391E−03 | 2.96 | 50 |
| 425907 | AA365752 | Hs. 155965 | ESTs | 3.236E−02 | 2.96 | 36.25 |
| 418679 | D38552 | Hs. 1191 | KIAA0073 protein | 4.611E−03 | 2.96 | 35.25 |
| 439776 | AL360140 | Hs. 176005 | Homo sapiens mRNA full length insert cDN | 2.008E−03 | 2.96 | 33.75 |
| 446006 | NM_004403 | Hs. 13530 | deafness, autosomal dominant 5 | 5.005E−03 | 2.96 | 43.5 |
| 434822 | AW076088 | Hs. 4187 | hypothetical protein 24636 | 3.115E−05 | 2.95 | 82 |
| 437275 | AW976035 | Hs. 292396 | ESTs, Weakly similar to A47582 B-cell gr | 1.084E−03 | 2.95 | 49.75 |
| 439601 | AB029032 | Hs. 6606 | KIAA1109 protein | 4.808E−03 | 2.95 | 36.25 |
| 403707 | | | Target Exon | 9.630E−03 | 2.95 | 33.75 |
| 437613 | R19892 | Hs. 10267 | MIL1 protein | 2.942E−03 | 2.95 | 30.5 |
| 458455 | AV648310 | Hs. 213488 | ESTs | 2.098E−03 | 2.95 | 46.25 |
| 450256 | AA286887 | Hs. 24724 | MFH-amplified sequences with leucine-ric | 3.687E−05 | 2.94 | 101 |
| 442053 | R35343 | Hs. 24968 | Human DNA sequence from clone RP1-233G16 | 1.689E−03 | 2.94 | 50 |
| 448569 | BE382657 | Hs. 21486 | signal transducer and activator of trans | 5.551E−08 | 2.94 | 419.75 |
| 414792 | BE314949 | Hs. 87128 | hypothetical protein FLJ23309 | 1.177E−06 | 2.93 | 86.25 |
| 427794 | AA709186 | Hs. 99070 | ESTs | 1.290E−02 | 2.93 | 45.25 |
| 446552 | AW470827 | Hs. 156241 | ESTs | 1.086E−04 | 2.92 | 92.5 |
| 441969 | AI733386 | Hs. 129194 | ESTs, Weakly similar to ALU1_HUMAN ALU S | 1.821E−04 | 2.92 | 65.75 |
| 451644 | N23235 | Hs. 30567 | ESTs, Weakly similar to B34087 hypotheti | 2.212E−05 | 2.92 | 52.25 |
| 423129 | L44396 | Hs. 124106 | Homo sapiens cDNA FLJ11941 fis, clone HE | 2.600E−03 | 2.92 | 46.75 |
| 418522 | AA605038 | Hs. 7149 | Homo sapiens cDNA: FLJ21950 fis, clone H | 4.043E−04 | 2.92 | 42 |
| 426279 | AI648520 | Hs. 169084 | tubby like protein 3 | 1.598E−02 | 2.92 | 35.75 |
| 409444 | H47933 | Hs. 33983 | ESTs, Weakly similar to ALU6_HUMAN ALU S | 5.389E−04 | 2.92 | 33.5 |
| 445939 | BE018658 | Hs. 141003 | Homo sapiens cDNA: FLJ21691 fis, clone C | 4.991E−02 | 2.92 | 32 |
| 417788 | AI436699 | Hs. 84928 | nuclear transcription factor Y, beta | 1.116E−02 | 2.92 | 30 |
| 415023 | AA932146 | Hs. 133494 | Homo sapiens clone TCCCIA00164 mRNA sequ | 4.037E−04 | 2.92 | 38 |
| 452866 | R26969 | Hs. 268016 | Homo sapiens cDNA: FLJ21243 fis, clone C | 1.845E−03 | 2.92 | 50.5 |
| 457000 | NM_006750 | Hs. 172278 | syntrophin, beta 2 (dystrophin-associate | 5.420E−03 | 2.92 | 30.25 |
| 423568 | NM_005256 | Hs. 129818 | growth arrest-specific 2 | 2.024E−04 | 2.92 | 84 |
| 454000 | AA040620 | Hs. 5672 | hypothetical protein AF140225 | 1.078E−02 | 2.92 | 75.25 |
| 409600 | AJ011679 | Hs. 55099 | rab6 GTPase activating protein (GAP and | 2.212E−05 | 2.91 | 56 |
| 432451 | AW972771 | Hs. 292471 | ESTs, Weakly similar to ALU1_HUMAN ALU S | 3.668E−02 | 2.91 | 38.75 |
| 419126 | AI810144 | Hs. 135276 | ESTs | 6.101E−03 | 2.91 | 35.75 |
| 428974 | AA442693 | Hs. 272006 | ESTs, Weakly similar to I38022 hypotheti | 7.753E−06 | 2.90 | 100 |
| 440945 | AW505345 | Hs. 7540 | f-box and leucine-rich repeat protein 3A | 5.624E−02 | 2.90 | 53.25 |
| 439024 | R96696 | Hs. 35598 | ESTs | 2.750E−02 | 2.90 | 91.25 |
| 426126 | AL118747 | Hs. 26691 | ESTs | 3.163E−04 | 2.90 | 57 |
| 428738 | NM_000380 | Hs. 192803 | xeroderma pigmentosum, complementation g | 3.499E−04 | 2.90 | 56.25 |
| 403743 | | | C1002604: gi\|8393668\|ref\|NP_058989.1\|kin | 7.116E−03 | 2.90 | 49.25 |
| 437108 | AA434054 | Hs. 80624 | hypothetical protein MGC2560 | 5.406E−03 | 2.90 | 47.5 |
| 407816 | AW500857 | Hs. 40137 | anaphase-promoting complex 1; meiotic ch | 1.969E−02 | 2.90 | 47.5 |
| 420683 | AA830168 | Hs. 271305 | ESTs | 1.481E−03 | 2.90 | 42.75 |
| 414429 | R51494 | Hs. 71818 | ESTs | 1.903E−02 | 2.90 | 37.25 |
| 426590 | AA617830 | Hs. 28310 | ESTs | 3.777E−03 | 2.90 | 34.75 |
| 408138 | AA535740 | Hs. 170263 | tumor protein p53-binding protein, 1 | 7.123E−03 | 2.90 | 32.75 |
| 437151 | AA745618 | | BANP homolog, SMAR1 homolog | 6.130E−02 | 2.89 | 35.5 |
| 418729 | AB028449 | Hs. 87889 | helicase-moi | 1.032E−04 | 2.89 | 65.25 |
| 447002 | BE242866 | Hs. 16933 | HepA-related protein | 9.175E−07 | 2.88 | 51.25 |
| 451582 | AI963026 | Hs. 289958 | ESTs, Weakly similar to putative p150 [H | 9.642E−03 | 2.88 | 49.25 |
| 456804 | AI421645 | Hs. 139851 | caveolin 2 | 3.448E−02 | 2.88 | 50.5 |
| 428695 | AI355647 | Hs. 189999 | purinergic receptor (family A group 5) | 6.035E−05 | 2.88 | 42.5 |
| 418248 | NM_005000 | Hs. 83916 | NM_005000*: Homo sapiens NADH dehydrogena | 3.032E−02 | 2.86 | 48 |
| 427547 | BE047653 | Hs. 119183 | ESTs, Weakly similar to ZN91_HUMAN ZINC | 1.034E−03 | 2.86 | 47 |
| 409509 | AL036923 | Hs. 322710 | ESTs | 9.289E−03 | 2.86 | 52.5 |
| 421718 | AL117574 | | Homo sapiens mRNA; cDNA DKFZp434L2221 (f | 3.120E−05 | 2.85 | 60.75 |
| 419438 | AA406400 | Hs. 12482 | glyceronephosphate O-acyltransferase | 7.982E−03 | 2.85 | 38 |
| 412019 | AA485890 | Hs. 69330 | Homo sapiens cDNA FLJ13835 fis, clone TH | 3.895E−05 | 2.85 | 65.75 |
| 427704 | AW971063 | Hs. 292882 | ESTs | 4.162E−02 | 2.85 | 78.25 |
| 422607 | Z45471 | Hs. 118684 | stromal cell-derived factor 2 | 1.086E−04 | 2.85 | 65.25 |
| 403330 | | | Target Exon | 3.674E−04 | 2.85 | 74.5 |
| 430280 | AA361258 | Hs. 237868 | interleukin 7 receptor | 2.353E−04 | 2.85 | 80.75 |
| 436100 | AA704806 | Hs. 143842 | ESTs, Weakly similar to 2004399A chromos | 3.034E−02 | 2.85 | 54.5 |
| 453041 | AI680737 | Hs. 289068 | Homo sapiens cDNA FLJ11918 fis, clone HE | 5.421E−03 | 2.84 | 53 |
| 408527 | AL135018 | Hs. 33074 | Homo sapiens, clone IMAGE: 3606519, mRNA, | 8.617E−03 | 2.83 | 42.5 |
| 429503 | AA394183 | Hs. 26873 | ESTs | 1.693E−03 | 2.82 | 42.25 |
| 445564 | AB028957 | Hs. 12896 | KIAA1034 protein | 5.859E−03 | 2.82 | 41.5 |
| 401197 | | | ENSP00000229263*: HSPC213. | 3.234E−02 | 2.82 | 40.5 |
| 409205 | AI952884 | Hs. 14832 | ESTs, Moderately similar to unnamed prot | 3.937E−03 | 2.82 | 38.5 |
| 443280 | AA299688 | Hs. 24183 | ESTs | 2.494E−03 | 2.82 | 41.5 |
| 408411 | C15118 | Hs. 322482 | hypothetical protein DKFZp566J2046 | 1.546E−06 | 2.82 | 115 |
| 417831 | H16423 | Hs. 82685 | CD47 antigen (Rh-related antigen, integr | 3.330E−04 | 2.81 | 58.5 |
| 433483 | AI926520 | Hs. 31016 | putative DNA binding protein | 1.902E−02 | 2.81 | 33 |
| 433902 | AW292820 | Hs. 144906 | ESTs | 6.835E−04 | 2.81 | 49.25 |
| 409132 | AJ224538 | Hs. 50732 | protein kinase, AMP-activated, beta 2 no | 3.408E−07 | 2.81 | 137.25 |
| 431976 | AA719001 | Hs. 291065 | ESTs | 8.638E−04 | 2.80 | 43.25 |
| 425836 | AW955696 | Hs. 90960 | ESTs | 9.808E−06 | 2.80 | 57.5 |
| 439773 | AI051313 | Hs. 143315 | ESTs | 3.918E−03 | 2.80 | 37 |
| 427399 | NM_014883 | Hs. 177664 | KIAA0914 gene product | 1.085E−03 | 2.80 | 84.5 |
| 420985 | X94703 | | RAB28, member RAS oncogene family | 1.925E−03 | 2.79 | 39 |
| 410800 | BE280421 | Hs. 94499 | ESTs | 2.227E−04 | 2.79 | 56 |
| 418549 | AA927177 | Hs. 86041 | CGG triplet repeat binding protein 1 | 4.244E−04 | 2.79 | 53.75 |
| 420339 | AW968259 | Hs. 186647 | ESTs | 1.399E−05 | 2.79 | 73 |
| 443352 | H70284 | Hs. 160152 | ESTs, Weakly similar to FPHU alpha-fetop | 1.186E−03 | 2.79 | 40.75 |
| 417018 | M16038 | Hs. 80887 | v-yes-1 Yamaguchi sarcoma viral related | 1.542E−02 | 2.79 | 59.5 |
| 423067 | AA321355 | Hs. 285401 | colony stimulating factor 2 receptor, be | 2.710E−03 | 2.79 | 34 |
| 458971 | AL119206 | Hs. 126257 | ESTs, Weakly similar to ALU1_HUMAN ALU S | 4.816E−03 | 2.79 | 77.25 |
| 438493 | AI130740 | Hs. 6241 | phosphoinositide-3-kinase, regulatory su | 4.492E−06 | 2.79 | 149.5 |
| 424755 | AB033094 | Hs. 152925 | KIAA1268 protein | 3.619E−07 | 2.78 | 83 |
| 436562 | H71937 | Hs. 322904 | ESTs, Weakly similar to I38022 hypotheti | 4.099E−03 | 2.78 | 95 |
| 421662 | NM_014141 | Hs. 106552 | cell recognition molecule Caspr2 | 1.133E−03 | 2.78 | 38.25 |
| 408190 | AB032963 | Hs. 43577 | ATPase, Class I, type 8B, member 2 | 7.488E−02 | 2.77 | 37.25 |
| 434500 | AF143877 | Hs. 215047 | Homo sapiens clone IMAGE: 113431 mRNA seq | 1.159E−02 | 2.77 | 34.5 |
| 445044 | AL137728 | Hs. 12258 | Homo sapiens mRNA; cDNA DKFZp434B0920 (f | 5.012E−03 | 2.77 | 43.25 |
| 429312 | AL133572 | Hs. 199009 | protein containing CXXC domain 2 | 2.948E−03 | 2.76 | 34.75 |
| 445106 | T10219 | Hs. 12329 | KIAA0697 protein | 3.330E−04 | 2.76 | 61 |
| 450919 | AA011616 | Hs. 269877 | ESTs | 4.466E−04 | 2.75 | 50 |
| 435767 | H73505 | Hs. 117874 | ESTs | 7.701E−02 | 2.75 | 61.75 |
| 435872 | AA701357 | Hs. 192759 | ESTs | 8.827E−05 | 2.75 | 128.25 |
| 434521 | NM_002267 | Hs. 3886 | karyopherin alpha 3 (importin alpha 4) | 6.838E−04 | 2.75 | 74 |
| 432134 | AI816782 | Hs. 122583 | hypothetical protein FLJ21934 | 6.838E−04 | 2.75 | 59.75 |
| 452057 | AW952005 | Hs. 14928 | hypothetical protein FLJ12903 | 2.353E−04 | 2.75 | 45.25 |
| 424381 | AA285249 | Hs. 146329 | protein kinase Chk2 | 1.969E−02 | 2.75 | 44.75 |
| 434963 | AW974957 | Hs. 288719 | Homo sapiens cDNA FLJ12142 fis, clone MA | 4.260E−03 | 2.75 | 43.75 |
| 411352 | NM_002890 | Hs. 758 | RAS p21 protein activator (GTPase activa | 1.037E−03 | 2.75 | 43 |
| 426416 | AW612744 | Hs. 169824 | killer cell lectin-like receptor subfami | 6.555E−03 | 2.75 | 41 |
| 449259 | AW452058 | Hs. 257519 | ESTs | 5.652E−04 | 2.75 | 40.25 |
| 436184 | BE154067 | Hs. 136660 | ESTs, Weakly similar to ZN91_HUMAN ZINC | 9.624E−03 | 2.75 | 40.25 |
| 439605 | AF086431 | Hs. 22380 | ESTs | 5.206E−03 | 2.75 | 34.75 |
| 404676 | | | Target Exon | 2.577E−02 | 2.75 | 30.75 |
| 443154 | H04360 | Hs. 24283 | ESTs, Moderately similar to reduced expr | 2.599E−03 | 2.75 | 48.5 |
| 440538 | W76332 | Hs. 79107 | mitogen-activated protein kinase 14 | 4.096E−03 | 2.74 | 31.75 |
| 413823 | AI341417 | Hs. 29406 | ESTs | 3.893E−07 | 2.74 | 78.25 |
| 413880 | AI660842 | Hs. 110915 | interleukin 22 receptor | 5.412E−05 | 2.73 | 63.25 |
| 409005 | AW299806 | Hs. 297256 | ESTs | 6.345E−03 | 2.73 | 37.25 |
| 423706 | U95218 | Hs. 131924 | G protein-coupled receptor 65 | 7.848E−04 | 2.73 | 40.25 |
| 433745 | AF075320 | Hs. 28980 | hypothetical protein FLJ14540 | 4.629E−03 | 2.73 | 38.5 |
| 459436 | AA323121 | | gb: EST25881 Cerebellum II Homo sapiens c | 1.298E−03 | 2.73 | 41.5 |
| 415668 | AW957684 | Hs. 306814 | hypothetical protein FLJ21889 | 2.664E−02 | 2.73 | 65.25 |
| 439462 | AL133026 | Hs. 6567 | Homo sapiens mRNA; cDNA DKFZp436C136 (fr | 2.006E−03 | 2.72 | 34 |
| 436741 | AA860163 | Hs. 291319 | ESTs | 8.293E−03 | 2.72 | 33.5 |
| 422722 | H74219 | Hs. 269772 | ESTs | 9.896E−04 | 2.72 | 40 |
| 447030 | AW444659 | Hs. 232184 | ESTs | 5.005E−03 | 2.71 | 30.75 |
| 446217 | AI651594 | Hs. 99709 | ESTs | 1.714E−07 | 2.70 | 116.25 |
| 413838 | AV661185 | Hs. 75574 | mitochondrial ribosomal protein L19 | 5.669E−04 | 2.70 | 44.25 |
| 445715 | AB012958 | Hs. 13137 | UV radiation resistance associated gene | 1.087E−04 | 2.70 | 54.75 |
| 411213 | AA676939 | Hs. 69285 | neuropilin 1 | 3.341E−03 | 2.70 | 53.75 |
| 412935 | BE267045 | Hs. 75064 | tubulin-specific chaperone c | 1.090E−04 | 2.70 | 47.75 |
| 408051 | AI623351 | Hs. 172148 | ESTs | 3.449E−02 | 2.70 | 41.75 |
| 457650 | AA649162 | Hs. 236456 | ESTs | 1.107E−05 | 2.69 | 80.25 |
| 422461 | NM_003417 | Hs. 117077 | zinc finger protein 264 | 7.112E−04 | 2.69 | 33.75 |
| 421524 | AA312082 | Hs. 105445 | GDNF family receptor alpha 1 | 1.572E−05 | 2.68 | 72 |
| 453779 | N35187 | Hs. 43388 | 28 kD interferon responsive protein | 9.788E−08 | 2.68 | 183 |
| 425284 | AF155568 | Hs. 348043 | NS1-associated protein 1 | 2.470E−04 | 2.68 | 44.5 |
| 451107 | AA235108 | | Homo sapiens ubiquitin protein ligase (U | 1.976E−05 | 2.68 | 45.75 |
| 443849 | BE566066 | Hs. 9893 | ASB-3 protein | 6.219E−04 | 2.68 | 48.25 |
| 438182 | AW342140 | Hs. 182545 | ESTs, Weakly similar to ALU1_HUMAN ALU S | 7.507E−04 | 2.67 | 47 |
| 433312 | AI241331 | Hs. 131765 | ESTs, Moderately similar to I38937 DNA/R | 1.822E−04 | 2.67 | 44.5 |
| 437838 | AI307229 | Hs. 184304 | ESTs | 2.389E−03 | 2.67 | 43.75 |
| 426797 | AW936258 | Hs. 342849 | ADP-ribosylation factor-like 5 | 3.447E−02 | 2.67 | 39.25 |
| 430594 | AK000790 | Hs. 246885 | hypothetical protein FLJ20783 | 1.713E−02 | 2.67 | 31 |
| 429245 | AW969785 | Hs. 285885 | Homo sapiens cDNA FLJ11321 fis, clone PL | 2.180E−02 | 2.67 | 30.75 |
| 407796 | AA195509 | Hs. 39733 | postsynaptic protein CRIPT | 9.005E−04 | 2.67 | 39.75 |
| 421939 | BE169531 | Hs. 109727 | TAK1-binding protein 2; KIAA0733 protein | 9.459E−04 | 2.67 | 83.25 |
| 429623 | NM_005308 | Hs. 211569 | G protein-coupled receptor kinase 5 | 4.267E−03 | 2.67 | 62.75 |
| 432485 | N90866 | Hs. 276770 | CDW52 antigen (CAMPATH-1 antigen) | 2.966E−07 | 2.67 | 203 |
| 451690 | AW451469 | Hs. 209990 | ESTs | 2.664E−02 | 2.67 | 36.75 |
| 411777 | BE067552 | | gb: MR4-BT0358-020200-002-g10 BT0358 Homo | 1.338E−02 | 2.67 | 43.75 |
| 420623 | BE245485 | Hs. 99437 | Homo sapiens mRNA; cDNA DKFZp586G1924 (f | 2.015E−03 | 2.66 | 48.25 |
| 450607 | AL050373 | Hs. 25213 | hypothetical protein | 3.968E−06 | 2.66 | 128.5 |
| 428466 | AF151063 | Hs. 184456 | hypothetical protein | 1.928E−03 | 2.66 | 42 |
| 424321 | W74048 | Hs. 1765 | lymphocyte-specific protein tyrosine kin | 3.892E−05 | 2.66 | 71.5 |
| 411943 | BE502436 | Hs. 7962 | ESTs, Weakly similar to S44608 C02F5.6 p | 8.939E−03 | 2.66 | 34.5 |
| 409161 | W07662 | Hs. 50861 | sirtuin (silent mating type information | 1.395E−05 | 2.66 | 45.25 |
| 407908 | BE379758 | Hs. 110853 | uncharacterized hematopoietic stem/proge | 4.698E−02 | 2.66 | 37.75 |
| 444057 | AA316896 | Hs. 257267 | FYVE and coiled-coil domain containing 1 | 1.487E−04 | 2.66 | 56 |
| 419216 | AU076718 | Hs. 164021 | small inducible cytokine subfamily B (Cy | 2.333E−02 | 2.66 | 36.5 |
| 427297 | AW292593 | Hs. 334907 | Homo sapiens, clone MGC: 17333, mRNA, com | 7.913E−05 | 2.66 | 90.25 |
| 446783 | AW138343 | Hs. 141867 | ESTs | 3.076E−03 | 2.66 | 36 |
| 427528 | AU077143 | Hs. 179565 | minichromosome maintenance deficient (S. | 7.282E−06 | 2.65 | 64.75 |
| 446830 | BE179030 | Hs. 239307 | Human DNA sequence from clone RP5-1174N9 | 2.019E−04 | 2.65 | 47.5 |
| 440529 | AW207640 | Hs. 16478 | Homo sapiens cDNA: FLJ21718 fis, clone C | 5.403E−04 | 2.65 | 40.5 |
| 434821 | AA159111 | Hs. 284281 | Human putative ribosomal protein S1 mRNA | 2.483E−05 | 2.65 | 50.25 |
| 437830 | AB020658 | Hs. 5867 | KIAA0851 protein; suppressor of actin 1 | 1.617E−03 | 2.65 | 58 |
| 418832 | X04011 | Hs. 88974 | cytochrome b-245, beta polypeptide (chro | 6.389E−05 | 2.64 | 56.25 |
| 445652 | AL117473 | Hs. 13036 | DKFZP727A071 protein | 7.158E−04 | 2.64 | 39 |
| 447387 | AI268331 | Hs. 102237 | tubby super-family protein | 7.552E−07 | 2.64 | 67.25 |
| 453315 | BE544203 | Hs. 24831 | ESTs | 3.624E−03 | 2.64 | 41.75 |
| 425266 | J00077 | Hs. 155421 | alpha-fetoprotein | 3.338E−03 | 2.64 | 43.75 |
| 414602 | AW630088 | Hs. 76550 | Homo sapiens mRNA; cDNA DKFZp564B1264 (f | 1.652E−04 | 2.63 | 86.5 |
| 436235 | AI084982 | Hs. 120790 | ESTs | 2.470E−04 | 2.63 | 36.25 |
| 430027 | AB023197 | Hs. 227743 | KIAA0980 protein | 9.843E−07 | 2.63 | 78.5 |
| 440528 | BE313555 | Hs. 7252 | KIAA1224 protein | 2.713E−03 | 2.63 | 50 |
| 403976 | | | Target Exon | 4.265E−03 | 2.63 | 49.75 |
| 447657 | AI953011 | Hs. 345287 | ESTs | 5.146E−04 | 2.63 | 34.75 |
| 412230 | AI810374 | Hs. 124177 | ESTs, Weakly similar to 2109260A B cell | 1.925E−03 | 2.62 | 31.25 |
| 407804 | AF228603 | Hs. 39957 | pleckstrin 2 (mouse) homolog | 5.947E−04 | 2.62 | 61.25 |
| 410853 | H04588 | Hs. 30469 | ESTs | 6.105E−03 | 2.62 | 76.5 |
| 416611 | AA568308 | | ESTs, Weakly similar to ALU6_HUMAN ALU S | 5.669E−04 | 2.62 | 49.75 |
| 452207 | NM_014517 | Hs. 28423 | upstream binding protein 1 (LBP-1a) | 1.037E−05 | 2.62 | 76.75 |
| 435445 | AA737345 | Hs. 294041 | ESTs | 2.706E−03 | 2.61 | 35.5 |
| 434608 | AA805443 | Hs. 179909 | hypothetical protein FLJ22995 | 1.133E−03 | 2.61 | 50 |
| 420058 | AK001423 | Hs. 94694 | Homo sapiens cDNA FLJ10561 fis, clone NT | 6.456E−06 | 2.61 | 79.75 |
| 447769 | AW873704 | Hs. 320831 | Homo sapiens cDNA FLJ14597 fis, clone NT | 4.253E−04 | 2.61 | 47.5 |
| 438441 | AW664960 | Hs. 205319 | ESTs | 2.024E−04 | 2.61 | 66 |
| 448770 | AA326683 | Hs. 21992 | likely ortholog of mouse variant polyade | 2.860E−04 | 2.61 | 69.5 |
| 431899 | AA521381 | Hs. 187726 | ESTs | 4.627E−03 | 2.61 | 37.75 |
| 449082 | BE387561 | Hs. 22981 | DKFZP586M1523 protein | 2.340E−04 | 2.60 | 52.5 |
| 449881 | Z28444 | Hs. 24119 | Homo sapiens mRNA; cDNA DKFZp586G2222 (f | 9.477E−04 | 2.60 | 66.5 |
| 433913 | AI694106 | Hs. 72325 | ESTs, Weakly similar to I38022 hypotheti | 6.131E−02 | 2.60 | 43.25 |
| 442432 | BE093589 | Hs. 38178 | hypothetical protein FLJ23468 | 1.031E−04 | 2.60 | 76.25 |
| 425118 | AU076611 | Hs. 154672 | methylene tetrahydrofolate dehydrogenase | 3.205E−03 | 2.60 | 59.25 |
| 422283 | AW411307 | Hs. 114311 | CDC45 (cell division cycle 45, S.cerevis | 1.561E−04 | 2.60 | 45.25 |
| 412520 | AA442324 | Hs. 795 | H2A histone family, member O | 1.669E−05 | 2.60 | 95.5 |
| 411580 | AL080088 | Hs. 70877 | DKFZP564K2062 protein | 7.455E−05 | 2.60 | 42.75 |
| 431562 | AI884334 | Hs. 11637 | ESTs | 9.075E−02 | 2.59 | 48.5 |
| 419426 | AI214690 | Hs. 346257 | aldo-keto reductase family 1, member B1 | 2.462E−04 | 2.59 | 48 |
| 422241 | Y00062 | Hs. 170121 | protein tyrosine phosphatase, receptor t | 3.968E−06 | 2.58 | 129 |
| 401928 | | | Target Exon | 4.841E−02 | 2.57 | 32 |
| 427130 | AB029020 | Hs. 173694 | KIAA1097 protein | 9.832E−06 | 2.57 | 66.5 |
| 409614 | BE297412 | Hs. 55189 | hypothetical protein | 9.799E−08 | 2.57 | 161.5 |
| 448873 | NM_003677 | Hs. 22393 | density-regulated protein | 3.845E−04 | 2.56 | 40.25 |
| 413305 | NM_000426 | Hs. 323511 | Homo sapiens cDNA: FLJ23176 fis, clone L | 1.835E−02 | 2.56 | 43.75 |
| 446771 | AA128965 | Hs. 60679 | TATA box binding protein (TBP)-associate | 1.873E−05 | 2.56 | 78.5 |
| 422392 | NM_005908 | Hs. 115945 | mannosidase, beta A, lysosomal | 1.898E−02 | 2.56 | 30 |
| 423968 | AF098277 | Hs. 136529 | solute carrier family 23 (nucleobase tra | 1.648E−06 | 2.55 | 74.25 |
| 418941 | AA452970 | Hs. 239527 | E1B-55 kDa-associated protein 5 | 3.034E−02 | 2.55 | 56.75 |
| 434669 | AF151534 | Hs. 92023 | core histone macroH2A2.2 | 2.738E−06 | 2.55 | 119.25 |
| 432476 | T94344 | Hs. 326263 | ESTs | 1.691E−03 | 2.55 | 93.5 |
| 452748 | AB011128 | Hs. 30512 | Homo sapiens mRNA for KIAA0556 protein, | 7.507E−04 | 2.55 | 46.5 |
| 445757 | AW449065 | Hs. 13264 | KIAA0856 protein | 8.934E−03 | 2.55 | 44.25 |
| 434948 | AI498469 | Hs. 12622 | ESTs, Highly similar to AF161436 1 HSPC3 | 2.011E−03 | 2.55 | 40 |
| 439372 | AF088033 | Hs. 159225 | ESTs | 1.200E−02 | 2.55 | 35.5 |
| 448888 | AW196663 | Hs. 200242 | caspase recruitment domain protein 6 | 1.915E−04 | 2.55 | 40.25 |
| 417821 | BE245149 | Hs. 82643 | protein tyrosine kinase 9 | 7.985E−03 | 2.54 | 66 |
| 415000 | AW025529 | Hs. 239812 | Homo sapiens serologically defined breas | 4.108E−05 | 2.54 | 74.75 |
| 417558 | AF045229 | Hs. 82280 | regulator of G-protein signalling 10 | 1.338E−02 | 2.54 | 37.75 |
| 418796 | AA228351 | Hs. 34060 | ESTs | 1.815E−04 | 2.54 | 38.5 |
| 450196 | AW956868 | Hs. 24608 | DKFZP564D177 protein | 1.175E−05 | 2.54 | 138.5 |
| 422043 | AL133649 | Hs. 110953 | retinoic acid induced 1 | 3.856E−04 | 2.54 | 51.5 |
| 428985 | AL134193 | Hs. 194709 | paraneoplastic antigen MA1 | 1.298E−03 | 2.53 | 42.5 |
| 411196 | W31212 | Hs. 69192 | vacuolar protein sorting 29 (yeast homol | 1.439E−02 | 2.53 | 41.25 |
| 427094 | AB025254 | Hs. 283761 | tudor repeat associator with PCTAIRE 2 | 2.092E−07 | 2.53 | 90.5 |
| 452737 | AK001680 | Hs. 30488 | DKFZP434F091 protein | 2.090E−03 | 2.52 | 30.5 |
| 407355 | AA846203 | Hs. 193974 | ESTs, Weakly similar to ALU1_HUMAN ALU S | 9.360E−04 | 2.52 | 30.5 |
| 431374 | BE258532 | Hs. 251871 | CTP synthase | 6.831E−04 | 2.52 | 72.75 |
| 427647 | W19744 | Hs. 180059 | Homo sapiens cDNA FLJ20653 fis, clone KA | 3.898E−05 | 2.52 | 73.5 |
| 407436 | AF211977 | | gb: Homo sapiens LENG10 mRNA, partial seq | 1.437E−02 | 2.52 | 30 |
| 437145 | AF007216 | Hs. 5462 | solute carrier family 4, sodium bicarbon | 1.134E−03 | 2.52 | 73.25 |
| 421446 | AA682425 | Hs. 118959 | ESTs | 1.774E−02 | 2.52 | 32.25 |
| 458079 | AI796870 | Hs. 54277 | DNA segment on chromosome X (unique) 992 | 3.091E−06 | 2.52 | 130.5 |
| 424375 | AF070547 | Hs. 146312 | Homo sapiens clone 24820 mRNA sequence | 4.423E−02 | 2.51 | 31 |
| 428727 | AF078847 | Hs. 191356 | general transcription factor IIH, polype | 9.891E−04 | 2.51 | 63.25 |
| 421405 | AA251944 | Hs. 104058 | CGI-29 protein | 1.567E−04 | 2.51 | 74.5 |
| 418733 | AA227714 | Hs. 179703 | KIAA0129 gene product | 1.650E−04 | 2.50 | 56 |
| 447225 | R62676 | Hs. 17820 | Rho-associated, coiled-coil containing p | 4.611E−05 | 2.50 | 95.75 |
| 433162 | AI025842 | Hs. 152530 | ESTs | 5.424E−05 | 2.50 | 102.75 |
| 423645 | AI215632 | Hs. 147487 | ESTs | 1.736E−04 | 2.50 | 56.75 |
| 446045 | AV656268 | Hs. 209153 | angiopoietin-like 3 | 2.239E−04 | 2.50 | 56.25 |
| 430273 | AI311127 | Hs. 125522 | ESTs | 9.035E−04 | 2.50 | 46 |
| 401898 | | | NM_024722*: Homo sapiens hypothetical pro | 1.927E−03 | 2.50 | 45 |
| 427210 | BE396283 | Hs. 173987 | eukaryotic translation initiation factor | 6.852E−03 | 2.50 | 44.25 |
| 445664 | AW968638 | Hs. 237691 | ESTs, Weakly similar to KIAA0601 protein | 7.093E−05 | 2.50 | 39.25 |
| 422195 | AB007903 | Hs. 113082 | KIAA0443 gene product | 6.581E−03 | 2.50 | 33 |
| 423069 | W15613 | Hs. 1613 | adenosine A2a receptor | 5.618E−02 | 2.50 | 30.25 |
| 417928 | AA209344 | Hs. 30177 | ESTs | 2.878E−04 | 2.49 | 68.5 |
| 448717 | R67419 | Hs. 21851 | Homo sapiens cDNA FLJ12900 fis, clone NT | 4.097E−03 | 2.49 | 50.25 |
| 433000 | U26710 | Hs. 3144 | Cas-Br-M (murine) ectropic retroviral tr | 2.036E−02 | 2.48 | 44.75 |
| 426011 | AW996096 | Hs. 58924 | ESTs, Weakly similar to JC5594 jerky gen | 5.152E−04 | 2.48 | 45.75 |
| 407112 | AA070801 | Hs. 51615 | ESTs, Weakly similar to ALU7_HUMAN ALU S | 3.450E−02 | 2.47 | 106.75 |
| 410196 | AI936442 | Hs. 59838 | hypothetical protein FLJ10808 | 4.868E−05 | 2.47 | 65.5 |
| 430487 | D87742 | Hs. 241552 | KIAA0268 protein | 2.235E−04 | 2.47 | 43.5 |
| 427254 | AL121523 | Hs. 97774 | ESTs | 4.813E−03 | 2.47 | 36.5 |
| 452480 | AI903526 | | gb: RC-BT031-090199-063 BT031 Homo sapien | 4.994E−02 | 2.47 | 31.75 |
| 403027 | | | C21000364*: gi\|8394509\|ref\|NP_058778.1\| u | 2.380E−03 | 2.47 | 34.25 |
| 414760 | BE298063 | Hs. 77254 | chromobox homolog 1 (Drosophila HP1 beta | 5.090E−07 | 2.47 | 79.5 |
| 444931 | AV652066 | Hs. 75113 | general transcription factor IIIA | 7.180E−04 | 2.47 | 70.5 |
| 437708 | AB033020 | Hs. 5801 | KIAA1194 protein | 8.210E−06 | 2.47 | 64.25 |
| 440340 | AW895503 | Hs. 125276 | ESTs | 8.596E−03 | 2.47 | 33.75 |
| 448198 | BE622100 | Hs. 209406 | ESTs, Weakly similar to I38600 zinc fing | 2.871E−04 | 2.46 | 43.75 |
| 405689 | | | NM_018850*: Homo sapiens ATP-binding cass | 5.010E−03 | 2.46 | 133 |
| 418203 | X54942 | Hs. 83758 | CDC28 protein kinase 2 | 1.692E−03 | 2.45 | 50.25 |
| 449239 | T24653 | Hs. 23360 | likely ortholog of yeast ARV1 | 1.272E−06 | 2.45 | 46.5 |
| 434128 | W93170 | Hs. 284164 | protein x 0004 | 2.354E−04 | 2.45 | 41 |
| 416354 | NM_000633 | Hs. 79241 | B-cell CLL/lymphoma 2 | 5.720E−05 | 2.45 | 40 |
| 438141 | AW946871 | | gb: RC2-ET0022-080500-012-d02 ET0022 Homo | 1.547E−03 | 2.45 | 33.5 |
| 423983 | AA333261 | | gb: EST37476 Embryo, 8 week I Homo sapien | 2.578E−02 | 2.45 | 31.25 |
| 438922 | R71288 | Hs. 259664 | ESTs | 5.297E−02 | 2.45 | 31.5 |
| 426295 | AW367283 | Hs. 278270 | zinc finger protein 6 (CMPX1) | 3.307E−05 | 2.45 | 169.5 |
| 420936 | AA456112 | Hs. 99410 | ESTs | 6.341E−03 | 2.45 | 36.5 |
| 438475 | W03856 | Hs. 13188 | ESTs, Highly similar to Gene product wit | 1.714E−07 | 2.44 | 157.5 |
| 446487 | AA195526 | Hs. 44625 | Rad50-interacting protein 1 | 2.288E−03 | 2.44 | 32.5 |
| 450253 | AL133047 | Hs. 24715 | Homo sapiens mRNA; cDNA DKFZp434D0215 (f | 4.265E−03 | 2.44 | 62 |
| 421999 | U50535 | Hs. 110630 | Human BRCA2 region, mRNA sequence CG006 | 3.673E−02 | 2.44 | 73.5 |
| 442643 | U82756 | Hs. 3991 | PRP4/STK/WD splicing factor | 4.032E−02 | 2.44 | 32.75 |
| 427156 | BE621719 | Hs. 173802 | KIAA0603 gene product | 2.841E−02 | 2.43 | 54.5 |
| 433201 | AB040896 | Hs. 21104 | KIAA1463 protein | 2.098E−03 | 2.43 | 45.75 |
| 442149 | AB014550 | Hs. 8118 | KIAA0650 protein | 2.829E−03 | 2.42 | 52.25 |
| 436021 | R26877 | Hs. 24128 | ESTs | 5.639E−03 | 2.42 | 38.5 |
| 451099 | R52795 | Hs. 25954 | interleukin 13 receptor, alpha 2 | 4.993E−02 | 2.42 | 52 |
| 430526 | AF181862 | Hs. 242407 | G protein-coupled receptor, family C, gr | 4.126E−05 | 2.42 | 101.25 |
| 414404 | W16712 | Hs. 306117 | KIAA0306 protein | 9.266E−05 | 2.42 | 94 |
| 410664 | NM_006033 | Hs. 65370 | lipase, endothelial | 2.192E−03 | 2.42 | 57 |
| 436643 | AA757626 | Hs. 10941 | ESTs, Weakly similar to IPP1_HUMAN PROTE | 2.235E−04 | 2.41 | 65.75 |
| 418780 | AA228078 | Hs. 255096 | ESTs | 2.829E−03 | 2.41 | 41 |
| 427468 | AB036829 | Hs. 178347 | SKIP for skeletal muscle and kidney enri | 2.122E−04 | 2.41 | 41 |
| 437143 | AW204056 | Hs. 8917 | ESTs | 1.969E−07 | 2.41 | 124.75 |
| 439747 | AK001148 | Hs. 6671 | COP9 complex subunit 4 | 2.349E−04 | 2.41 | 53 |
| 447881 | BE620886 | Hs. 75354 | GCN1 (general control of amino-acid synt | 4.442E−03 | 2.41 | 54 |
| 443562 | AF118838 | Hs. 9599 | solute carrier family 25, member 13 (cit | 5.142E−02 | 2.41 | 53.25 |
| 448280 | AW014215 | Hs. 357 | zinc finger protein 134 (clone pHZ-15) | 9.001E−04 | 2.41 | 37 |
| 439645 | BE091801 | Hs. 27167 | ESTs, Weakly similar to I38022 hypotheti | 5.430E−07 | 2.41 | 71.5 |
| 427657 | AV652249 | Hs. 180107 | polymerase (DNA directed), beta | 2.417E−07 | 2.41 | 116.5 |
| 412977 | AA125910 | Hs. 191461 | ESTs | 6.492E−04 | 2.41 | 55.25 |
| 450203 | AF097994 | Hs. 301528 | L-kynurenine/alpha-aminoadipate aminotra | 5.298E−02 | 2.40 | 47.75 |
| 417089 | H52280 | Hs. 18612 | Homo sapiens cDNA: FLJ21909 fis, clone H | 1.131E−07 | 2.40 | 155.5 |
| 446044 | H67567 | Hs. 13572 | calcium modulating ligand | 6.632E−07 | 2.40 | 91 |
| 444088 | AW297946 | Hs. 138208 | ESTs | 3.911E−02 | 2.40 | 40.5 |
| 449118 | R67477 | Hs. 23103 | Bet1 (S. cerevisiae) homolog | 7.679E−03 | 2.40 | 39.25 |
| 441488 | AW450935 | Hs. 7862 | hypothetical protein FLJ20312 | 1.044E−05 | 2.40 | 102.25 |
| 424088 | AL049942 | Hs. 139240 | DKFZP564F1422 protein | 3.007E−04 | 2.39 | 42.5 |
| 444755 | AA431791 | Hs. 113823 | CIpX (caseinolytic protease X, E. coli) | 2.406E−02 | 2.39 | 33 |
| 439768 | AI337300 | Hs. 173138 | hypothetical protein MGC4604 | 1.541E−06 | 2.39 | 98.5 |
| 427985 | AI770170 | Hs. 29643 | Homo sapiens cDNA FLJ13103 fis, clone NT | 4.814E−03 | 2.39 | 42.25 |
| 448646 | AU077149 | Hs. 21704 | transcription factor 12 (HTF4, helix-loo | 3.170E−04 | 2.38 | 83.75 |
| 449845 | AW971183 | Hs. 9683 | DnaJ (Hsp40) homolog, subfamily C, membe | 1.829E−04 | 2.38 | 111 |
| 451578 | NM_016323 | Hs. 26663 | cyclin-E binding protein 1 | 3.893E−07 | 2.38 | 146.25 |
| 446570 | AV659177 | Hs. 127160 | ESTs | 1.399E−05 | 2.38 | 79.5 |
| 430532 | D61216 | Hs. 18672 | ESTs | 1.599E−02 | 2.38 | 35.25 |
| 418822 | Z43371 | Hs. 7012 | ESTs | 5.219E−03 | 2.38 | 40.75 |
| 446507 | AA352554 | Hs. 15164 | nuclear DNA-binding protein | 3.630E−03 | 2.38 | 58 |
| 419203 | AA488719 | Hs. 190151 | ESTs | 2.387E−03 | 2.38 | 56.25 |
| 435427 | AA682573 | Hs. 188982 | ESTs, Weakly similar to organic anion tr | 2.012E−03 | 2.38 | 73.5 |
| 444372 | AW377983 | Hs. 298140 | Homo sapiens cDNA: FLJ22502 fis, clone H | 1.269E−04 | 2.38 | 31 |
| 414496 | W73853 | | ESTs | 8.625E−04 | 2.38 | 73 |
| 430219 | X99209 | Hs. 235887 | HMT1 (hnRNP methyltransferase, S. cerevi | 2.494E−02 | 2.38 | 42 |
| 447211 | AL161961 | Hs. 17767 | KIAA1554 protein | 1.716E−07 | 2.38 | 143.5 |
| 427020 | AA397546 | Hs. 119151 | ESTs | 2.493E−03 | 2.38 | 35.25 |
| 425375 | AA631977 | Hs. 155995 | KIAA0643 protein | 1.240E−05 | 2.38 | 34.25 |
| 440341 | AW664012 | Hs. 132333 | ESTs | 6.525E−04 | 2.38 | 30.25 |
| 418300 | AI433074 | Hs. 86682 | Homo sapiens cDNA: FLJ21578 fis, clone C | 3.787E−02 | 2.38 | 30.25 |
| 429978 | AA249027 | | ribosomal protein S6 | 2.953E−03 | 2.37 | 85.25 |
| 422614 | AI908006 | Hs. 295362 | Homo sapiens cDNA FLJ14459 fis, clone HE | 5.423E−03 | 2.37 | 35.25 |
| 447279 | AA325308 | Hs. 18016 | Homo sapiens mRNA; cDNA DKFZp586H0324 (f | 1.694E−03 | 2.37 | 47.5 |
| 420613 | AI873871 | Hs. 122444 | ESTs, Weakly similar to A47582 B-cell gr | 4.453E−07 | 2.37 | 89.5 |
| 408521 | AA055264 | Hs. 260848 | ESTs, Weakly similar to S23650 retroviru | 1.971E−05 | 2.37 | 44.75 |
| 429617 | X89984 | Hs. 211563 | B-cell CLL/lymphoma 7A | 3.335E−03 | 2.36 | 30 |
| 414172 | AW954324 | Hs. 75790 | phosphatidylinositol glycan, class C | 8.270E−03 | 2.36 | 31 |
| 418416 | U11700 | Hs. 84999 | ATPase, Cu transporting, beta polypeptid | 1.764E−05 | 2.36 | 59.25 |
| 446071 | N51527 | Hs. 13659 | hypothetical protein DKFZp586F2423 | 8.224E−06 | 2.36 | 102.75 |
| 451743 | AW074266 | Hs. 23071 | ESTs | 9.290E−05 | 2.36 | 56.25 |
| 437134 | AA349944 | Hs. 42915 | ARP2 (actin-related protein 2, yeast) ho | 1.199E−02 | 2.36 | 51.5 |
| 410612 | AW502698 | Hs. 118152 | ESTs | 3.734E−06 | 2.35 | 117.5 |
| 442439 | U09759 | Hs. 246857 | mitogen-activated protein kinase 9 | 7.907E−05 | 2.35 | 70 |
| 439103 | AF085959 | Hs. 38705 | ESTs | 1.616E−03 | 2.35 | 41.5 |
| 427469 | AA403084 | Hs. 269347 | ESTs, Weakly similar to 2109260A B cell | 4.093E−03 | 2.35 | 40.75 |
| 428044 | AA093322 | Hs. 301404 | RNA binding motif protein 3 | 6.130E−02 | 2.35 | 35.75 |
| 420137 | AA306478 | Hs. 95327 | CD3D antigen, delta polypeptide (TiT3 co | 2.255E−06 | 2.35 | 78.5 |
| 446594 | AI311917 | Hs. 16292 | ESTs | 5.144E−02 | 2.35 | 71.75 |
| 452685 | AI634651 | Hs. 30250 | v-maf musculoaponeurotic fibrosarcoma (a | 7.085E−07 | 2.34 | 145.75 |
| 439708 | AI761369 | Hs. 59584 | hypothetical protein FLJ21144 | 1.735E−04 | 2.34 | 36.5 |
| 451589 | AA424791 | Hs. 5734 | meningioma expressed antigen 5 (hyaluron | 3.852E−04 | 2.33 | 57 |
| 414312 | AA155694 | Hs. 191060 | ESTs | 7.918E−05 | 2.32 | 54.75 |
| 439556 | AI623752 | Hs. 163603 | ESTs | 4.627E−03 | 2.32 | 45.5 |
| 421025 | AW958975 | Hs. 29397 | Homo sapiens cDNA FLJ13226 fis, clone OV | 5.395E−04 | 2.32 | 37 |
| 435919 | AI052189 | Hs. 114104 | ESTs | 5.010E−03 | 2.32 | 30.75 |
| 408931 | AA251995 | Hs. 334648 | poly(A) polymerase alpha | 2.841E−02 | 2.32 | 37.25 |
| 438000 | AI825880 | Hs. 5985 | non-kinase Cdc42 effector protein SPEC2 | 6.103E−03 | 2.32 | 72.5 |
| 402041 | | | C15001201*: gi\|6841178\|gb\|AAF28942.1\|AF16 | 1.687E−03 | 2.32 | 40.5 |
| 437223 | C15105 | Hs. 330716 | Homo sapiens cDNA FLJ14368 fis, clone HE | 8.248E−04 | 2.31 | 95.25 |
| 420717 | AA284447 | Hs. 271887 | ESTs | 7.277E−02 | 2.31 | 41 |
| 408340 | AB037762 | Hs. 44268 | myelin gene expression factor 2 | 5.994E−05 | 2.31 | 39 |
| 452248 | AA093668 | Hs. 28578 | muscleblind (Drosophila)-like | 2.634E−05 | 2.31 | 62.25 |
| 436252 | AI539519 | Hs. 120969 | Homo sapiens cDNA FLJ11562 fis, clone HE | 5.406E−04 | 2.31 | 70 |
| 420825 | AI656727 | | gb: tt53f12.x1 NCI_CGAP_GC6 Homo sapiens | 7.165E−04 | 2.31 | 42.5 |
| 442831 | AI798959 | Hs. 131686 | ESTs | 7.169E−04 | 2.31 | 76.75 |
| 407970 | AW403814 | Hs. 41714 | BCL2-associated athanogene | 1.038E−02 | 2.30 | 30 |
| 410726 | AI623859 | Hs. 15936 | ESTs | 2.595E−04 | 2.30 | 33.25 |
| 437109 | AW006781 | Hs. 5457 | hypothetical protein FLJ10738 | 1.732E−04 | 2.30 | 45.25 |
| 415662 | AW972481 | Hs. 170610 | ESTs, Highly similar to G01887 MEK kinas | 6.380E−05 | 2.30 | 76.5 |
| 425913 | AA365799 | Hs. 50785 | SEC22, vesicle trafficking protein (S. c | 6.393E−05 | 2.30 | 79.75 |
| 410017 | AW952426 | Hs. 109438 | Homo sapiens clone 24775 mRNA sequence | 1.089E−04 | 2.30 | 82.25 |
| 422900 | M641201 | Hs. 222051 | ESTs | 5.657E−04 | 2.30 | 54.75 |
| 420164 | AW339037 | Hs. 24908 | ESTs | 2.491E−02 | 2.30 | 33.75 |
| 413029 | AL119399 | Hs. 293850 | ESTs | 2.764E−05 | 2.30 | 44.5 |
| 457605 | AV657778 | Hs. 3314 | selenoprotein P, plasma, 1 | 7.165E−04 | 2.30 | 82.25 |
| 407656 | AW747986 | Hs. 37443 | Homo sapiens mRNA; cDNA DKFZp434B2119 (f | 3.664E−04 | 2.29 | 45 |
| 452852 | AK001972 | Hs. 30822 | hypothetical protein FLJ11110 | 4.496E−06 | 2.29 | 121.5 |
| 456563 | AA989220 | Hs. 766 | ESTs | 1.715E−02 | 2.29 | 75 |
| 449103 | T24968 | Hs. 23038 | HSPC071 protein | 9.789E−05 | 2.29 | 53.75 |
| 425960 | AW410646 | Hs. 164649 | hypothetical protein DKFZp434H247 | 2.493E−03 | 2.29 | 52.75 |
| 417386 | AL037228 | Hs. 82043 | Dt23 gene product | 2.841E−02 | 2.28 | 37.25 |
| 443189 | AB023179 | Hs. 9059 | KIAA0962 protein | 9.877E−04 | 2.28 | 38.5 |
| 439815 | AA206079 | Hs. 6693 | hypothetical protein FLJ20420 | 8.804E−05 | 2.28 | 46.5 |
| 404298 | | | C6001238*: gi\|121715\|sp\|P26697\|GTA3_CHICK | 5.420E−03 | 2.28 | 42 |
| 441664 | AW748420 | Hs. 6236 | Homo sapiens cDNA: FLJ21487 fis, clone C | 9.882E−04 | 2.28 | 58.5 |
| 448959 | AI610343 | Hs. 186355 | ESTs | 2.020E−04 | 2.27 | 46 |
| 417206 | AA291183 | Hs. 81648 | hypothetical protein FLJ11021 similar to | 1.184E−03 | 2.27 | 40.75 |
| 422460 | AW445014 | Hs. 197746 | ESTs | 2.659E−02 | 2.27 | 33.5 |
| 448410 | AK000227 | Hs. 21126 | hypothetical protein FLJ20220 | 6.881E−02 | 2.27 | 32.75 |
| 424528 | AW073971 | Hs. 238954 | ESTs, Weakly similar to KIAA1204 protein | 6.225E−04 | 2.27 | 45.25 |
| 456721 | AA533356 | | gb: nj67f10.s1 NCI_CGAP_Pr10 Homo sapiens | 1.038E−02 | 2.27 | 30.25 |
| 416269 | AA177138 | Hs. 161671 | ESTs | 6.350E−03 | 2.27 | 47.75 |
| 430308 | BE540865 | Hs. 238990 | cyclin-dependent kinase inhibitor 1B (p2 | 1.398E−05 | 2.27 | 105 |
| 446161 | AA628206 | Hs. 14125 | p53 regulated PA26 nuclear protein | 4.251E−04 | 2.27 | 74.75 |
| 414256 | AW410035 | Hs. 75862 | MAD (mothers against decapentaplegic, Dr | 1.968E−02 | 2.27 | 37.5 |
| 457819 | AA057484 | Hs. 35406 | ESTs, Highly similar to unnamed protein | 2.574E−02 | 2.27 | 30.5 |
| 420463 | D51872 | Hs. 23492 | ESTs | 4.267E−03 | 2.27 | 33 |
| 408077 | AL133574 | Hs. 42458 | Homo sapiens mRNA; cDNA DKFZp586C1817 (f | 5.869E−03 | 2.27 | 75 |
| 401403 | | | Target Exon | 3.235E−02 | 2.26 | 59.75 |
| 440675 | AW005054 | Hs. 47883 | ESTs, Weakly similar to KCC1_HUMAN CALCI | 5.063E−06 | 2.26 | 163 |
| 417867 | AW952547 | Hs. 194603 | ESTs, Moderately similar to I38022 hypot | 7.964E−03 | 2.26 | 39.25 |
| 448356 | AL120837 | Hs. 20993 | high-glucose-regulated protein 8 | 3.340E−02 | 2.25 | 32 |
| 421931 | NM_000814 | Hs. 1440 | gamma-aminobutyric acid (GABA) A recepto | 2.471E−04 | 2.25 | 74.25 |
| 435524 | AK000001 | Hs. 4914 | Homo sapiens mRNA for FLJ00031 protein, | 4.354E−05 | 2.25 | 49.5 |
| 439475 | AA836331 | Hs. 134981 | ESTs | 4.626E−03 | 2.25 | 41 |
| 414436 | U50078 | Hs. 76127 | hect (homologous to the E6-AP (UBE3A) ca | 9.997E−03 | 2.25 | 62.25 |
| 436856 | AI469355 | Hs. 127310 | ESTs | 8.090E−07 | 2.25 | 210.5 |
| 407151 | H25836 | Hs. 301527 | ESTs, Moderately similar to unknown [H.s | 2.111E−07 | 2.25 | 231.25 |
| 435756 | AI418466 | Hs. 33665 | ESTs | 3.491E−04 | 2.25 | 56.25 |
| 416754 | H07145 | Hs. 6799 | ESTs, Weakly similar to T12483 hypotheti | 1.690E−03 | 2.25 | 46.75 |
| 444647 | H14718 | Hs. 11506 | Human clone 23589 mRNA sequence | 2.255E−02 | 2.25 | 45 |
| 440638 | AI376551 | | gb: te64e10.x1 Soares_NFL_T_GBC_S1 Homo s | 3.480E−03 | 2.25 | 36.25 |
| 429025 | AI399910 | Hs. 266782 | KIAA1826 protein | 3.338E−03 | 2.25 | 35.25 |
| 418383 | AA218986 | Hs. 118854 | ESTs | 3.769E−03 | 2.25 | 32.25 |
| 438829 | AA826926 | Hs. 204214 | ESTs, Weakly similar to I38022 hypotheti | 2.600E−03 | 2.25 | 42.75 |
| 416240 | NM_001981 | Hs. 79095 | epidermal growth factor receptor pathway | 2.219E−05 | 2.25 | 69.75 |
| 438030 | X98427 | Hs. 122634 | ESTs | 1.243E−03 | 2.24 | 213.75 |
| 419200 | AW966405 | Hs. 288856 | prefoldin 5 | 3.897E−07 | 2.24 | 136.25 |
| 433713 | AW976511 | Hs. 112592 | ESTs | 8.288E−03 | 2.24 | 91 |
| 447109 | X69086 | Hs. 286161 | Homo sapiens cDNA FLJ13613 fis. clone PL | 3.777E−03 | 2.24 | 52 |
| 452870 | AW502761 | Hs. 30909 | KIAA0430 gene product | 1.246E−02 | 2.24 | 31.25 |
| 427268 | X78520 | Hs. 174139 | chloride channel 3 | 1.922E−04 | 2.24 | 87.25 |
| 439046 | AA947354 | | gb: od86e11.s1 NCI_CGAP_Ov2 Homo sapiens | 1.488E−04 | 2.24 | 39.25 |
| 453929 | AW190054 | | gb: xl11d05.x1 NCI_CGAP_Ut4 Homo sapiens | 1.657E−02 | 2.23 | 30.25 |
| 417944 | AU077196 | Hs. 82985 | collagen, type V, alpha 2 | 3.331E−04 | 2.23 | 129.75 |
| 417303 | NM_001698 | Hs. 81886 | AU RNA-binding protein/enoyl-Coenzyme A | 3.330E−04 | 2.23 | 67 |
| 411495 | AP000693 | Hs. 70359 | KIAA0136 protein | 2.411E−02 | 2.23 | 36.25 |
| 452144 | AA032197 | Hs. 102558 | Homo sapiens, clone MGC: 5352, mRNA, comp | 2.789E−05 | 2.22 | 58.25 |
| 405270 | | | NM_018850*: Homo sapiens ATP-binding cass | 1.389E−02 | 2.22 | 58 |
| 452436 | BE077546 | Hs. 31447 | ESTs, Moderately similar to A46010 X-lin | 2.491E−03 | 2.22 | 40.25 |
| 450314 | AA574309 | Hs. 283402 | TCR eta | 2.286E−03 | 2.22 | 38 |
| 442806 | AW294522 | Hs. 149991 | ESTs | 3.790E−02 | 2.22 | 34.75 |
| 406274 | | | Target Exon | 1.084E−03 | 2.22 | 69.5 |
| 449437 | AI702038 | Hs. 100057 | Homo sapiens cDNA: FLJ22902 fis, clone K | 1.922E−04 | 2.22 | 73.75 |
| 434260 | AF121856 | Hs. 284291 | sorting nexin 6 | 1.321E−05 | 2.22 | 100.5 |
| 445893 | AI610702 | Hs. 28212 | ESTs, Weakly similar to TRHY_HUMAN TRICH | 2.942E−05 | 2.22 | 63.75 |
| 429520 | AA160142 | Hs. 205058 | hypothetical protein FLJ20075 | 1.839E−02 | 2.22 | 39 |
| 437801 | AA613866 | Hs. 5848 | Homo sapiens mRNA; cDNA DKFZp564L222 (fr | 4.426E−02 | 2.22 | 39 |
| 425508 | AA991551 | Hs. 97013 | Homo sapiens, Similar to RIKEN cDNA 2310 | 3.290E−05 | 2.21 | 59.25 |
| 425303 | AA354785 | | gb: EST63098 Jurkat T-cells V Homo sapien | 5.450E−02 | 2.21 | 30.75 |
| 450157 | AW961576 | Hs. 60178 | ESTs | 3.074E−03 | 2.20 | 69 |
| 453927 | AA082465 | Hs. 125031 | choline/ethanolaminephosphotransferase | 9.265E−06 | 2.20 | 58 |
| 437580 | AA761075 | Hs. 293567 | ESTs | 4.094E−03 | 2.20 | 54.75 |
| 408990 | AL022395 | Hs. 49526 | f-box and leucine-rich repeat protein 4 | 2.289E−03 | 2.20 | 44 |
| 438619 | AB032773 | Hs. 6341 | TU12B1-TY protein | 1.076E−02 | 2.20 | 39 |
| 404149 | | | C6002509*: gi\|5031885\|ref\|NP_005568.1\|li | 5.296E−02 | 2.20 | 36.25 |
| 448102 | AI750793 | Hs. 20295 | CHK1 (checkpoint, S. pombe) homolog | 1.901E−02 | 2.20 | 30 |
| 433618 | AA602539 | Hs. 345494 | ESTs | 4.698E−02 | 2.20 | 30.25 |
| 446591 | H44186 | Hs. 15456 | PDZ domain containing 1 | 5.821E−07 | 2.20 | 113.5 |
| 416041 | AA345547 | Hs. 53263 | hypothetical protein FLJ13287 | 5.649E−03 | 2.20 | 57.75 |
| 414588 | AA302905 | | gb: EST10607 Adipose tissue, white I Homo | 4.681E−04 | 2.20 | 38.25 |
| 459680 | H96982 | Hs. 42321 | ESTs | 3.860E−04 | 2.20 | 84.25 |
| 452574 | AF127481 | Hs. 301946 | lymphoid blast crisis oncogene | 1.483E−03 | 2.19 | 49 |
| 412240 | H72176 | Hs. 4273 | hypothetical protein FLJ13159 | 2.713E−03 | 2.19 | 33.25 |
| 423996 | AF205071 | Hs. 137425 | solute carrier family 21 (organic anion | 8.139E−02 | 2.19 | 50.75 |
| 440113 | AI916532 | Hs. 188272 | ESTs | 1.078E−03 | 2.19 | 49.5 |
| 456073 | AA587775 | Hs. 66295 | multi-PDZ-domain-containing protein | 1.316E−05 | 2.19 | 56 |
| 419675 | AL079310 | Hs. 92260 | high-mobility group protein 2-like 1 | 1.182E−03 | 2.18 | 50.25 |
| 427631 | AB029023 | Hs. 179946 | KIAA1100 protein | 5.139E−05 | 2.18 | 73 |
| 442332 | AI693251 | Hs. 8248 | Target CAT | 1.650E−04 | 2.18 | 91.5 |
| 450969 | BE244603 | Hs. 25726 | transposon-derived Buster 1 transposase-l | 2.101E−03 | 2.18 | 45 |
| 421077 | AK000061 | Hs. 101590 | hypothetical protein | 6.854E−03 | 2.17 | 46.5 |
| 444291 | AI598022 | Hs. 193989 | TAR DNA binding protein | 2.232E−04 | 2.17 | 40 |
| 407904 | W44735 | Hs. 9286 | Homo sapiens cDNA: FLJ21278 fis, clone C | 1.647E−04 | 2.17 | 35.5 |
| 442129 | N36918 | Hs. 20142 | PNAS-127 protein | 2.948E−03 | 2.17 | 34 |
| 412025 | AI827451 | Hs. 24143 | Wiskott-Aldrich syndrome protein interac | 3.489E−05 | 2.17 | 82.75 |
| 412059 | AA317962 | Hs. 249721 | ESTs, Moderately similar to PC4259 ferri | 3.674E−02 | 2.17 | 42 |
| 432235 | AA531129 | Hs. 190297 | ESTs | 5.460E−02 | 2.17 | 36.75 |
| 424247 | X14008 | Hs. 234734 | lysozyme (renal amyloidosis) | 8.638E−04 | 2.17 | 158.25 |
| 413326 | H88621 | Hs. 19762 | ESTs, Weakly similar to KIAA1140 protein | 7.992E−03 | 2.17 | 45.75 |
| 416065 | BE267931 | Hs. 78996 | proliferating cell nuclear antigen | 2.240E−04 | 2.17 | 123.25 |
| 413012 | D83777 | Hs. 75137 | KIAA0193 gene product | 2.948E−03 | 2.16 | 53.75 |
| 442092 | AW578669 | | hypothetical protein FLJ12439 | 1.619E−03 | 2.16 | 31.25 |
| 423630 | AB011132 | Hs. 129952 | KIAA0560 gene product | 3.203E−03 | 2.16 | 40.75 |
| 421921 | H83363 | Hs. 6820 | translocase of inner mitochondrial membr | 4.039E−04 | 2.15 | 52.25 |
| 429586 | T73510 | Hs. 209153 | angiopoietin-like 3 | 4.604E−05 | 2.15 | 132 |
| 449217 | AA278536 | Hs. 23262 | ribonuclease, RNase A family, k6 | 7.916E−02 | 2.15 | 45.5 |
| 409518 | BE384836 | Hs. 3454 | KIAA1821 protein | 8.314E−05 | 2.15 | 34.25 |
| 414525 | C14904 | Hs. 45184 | Homo sapiens cDNA FLJ12284 fis, clone MA | 4.233E−04 | 2.15 | 44 |
| 411979 | X85134 | Hs. 72984 | retinoblastoma-binding protein 5 | 9.468E−04 | 2.15 | 42.75 |
| 448705 | H05072 | Hs. 124984 | ESTs, Moderately similar to ALU7_HUMAN A | 2.180E−02 | 2.15 | 37.25 |
| 419165 | AW860767 | Hs. 118879 | ESTs | 1.038E−02 | 2.15 | 36 |
| 419479 | AI288348 | Hs. 23450 | mitochondrial ribosomal protein S25 | 8.590E−02 | 2.15 | 35.5 |
| 432709 | H17238 | | gb: ym42f03.r1 Soares infant brain 1NIB H | 1.353E−03 | 2.15 | 33.5 |
| 440266 | AA088809 | Hs. 19525 | hypothetical protein FLJ22794 | 9.808E−05 | 2.15 | 82.5 |
| 440010 | AA534930 | Hs. 127236 | hypothetical protein FLJ12879 | 1.619E−03 | 2.15 | 46.25 |
| 408866 | AW292096 | Hs. 255036 | ESTs | 4.439E−03 | 2.14 | 43.5 |
| 438023 | AF204883 | Hs. 6048 | FEM-1 (C. elegans) homolog b | 3.849E−04 | 2.14 | 50 |
| 439318 | AW837046 | Hs. 6527 | G protein-coupled receptor 56 | 3.331E−04 | 2.14 | 68.75 |
| 403576 | | | C3000124: gi\|12737057\|ref\|XP_012129.1\|si | 5.416E−03 | 2.14 | 35 |
| 429567 | R35606 | Hs. 326800 | Human EST clone 53125 mariner transposon | 3.623E−03 | 2.13 | 32 |
| 452679 | Z42387 | Hs. 83883 | transmembrane, prostate androgen induced | 3.069E−03 | 2.13 | 32 |
| 424904 | AI221739 | Hs. 96899 | ESTs | 1.035E−02 | 2.13 | 38.75 |
| 441991 | AW844404 | Hs. 126901 | Homo sapiens mRNA full length insert cDN | 3.860E−04 | 2.13 | 45.25 |
| 433160 | AW207002 | Hs. 134342 | TASP for testis-specific adriamycin sens | 1.543E−02 | 2.13 | 58.25 |
| 409969 | AW514668 | Hs. 194258 | ESTs, Moderately similar to ALU5_HUMAN A | 2.333E−02 | 2.13 | 34.75 |
| 437796 | AW407459 | Hs. 5836 | mitochondrial ribosomal protein S23 | 2.218E−05 | 2.13 | 57 |
| 404240 | | | NM_018950: Homo sapiens major histocompat | 6.450E−06 | 2.13 | 177.25 |
| 413587 | AA156164 | Hs. 286241 | protein kinase, cAMP-dependent, regulato | 1.842E−03 | 2.13 | 53.5 |
| 453022 | AA031499 | Hs. 118489 | ESTs | 1.483E−03 | 2.13 | 76 |
| 407183 | AA358015 | | gb: EST66864 Fetal lung III Homo sapiens | 5.708E−05 | 2.13 | 49.25 |
| 408047 | AW205461 | Hs. 243612 | ESTs | 6.105E−03 | 2.13 | 40.5 |
| 445800 | AA126419 | Hs. 32944 | inositol polyphosphate-4-phosphatase, ty | 9.289E−03 | 2.13 | 40 |
| 401952 | | | Target Exon | 3.784E−02 | 2.13 | 31.5 |
| 407874 | AI766311 | Hs. 289047 | Homo sapiens cDNA FLJ14059 fis, clone HE | 1.361E−06 | 2.12 | 156.25 |
| 430399 | AI916284 | Hs. 199671 | ESTs | 4.676E−04 | 2.12 | 62.25 |
| 444913 | AI362726 | Hs. 193656 | Homo sapiens mRNA for KIAA1658 protein, | 2.218E−05 | 2.12 | 42.5 |
| 412760 | AW379030 | Hs. 41324 | ESTs | 4.117E−05 | 2.11 | 55.75 |
| 443804 | AL135352 | Hs. 255883 | ESTs, Weakly similar to I38022 hypotheti | 9.035E−04 | 2.11 | 44 |
| 454146 | BE086548 | Hs. 42346 | calcineurin-binding protein calsarcin-1 | 1.848E−03 | 2.11 | 115 |
| 425745 | U44060 | Hs. 14427 | Homo sapiens cDNA: FLJ21800 fis, clone H | 6.541E−04 | 2.11 | 81 |
| 426676 | R19549 | Hs. 13996 | Homo sapiens cDNA: FLJ23260 fis, clone C | 1.640E−06 | 2.11 | 147.75 |
| 430587 | AK000341 | Hs. 246107 | elongation of very long chain fatty acid | 1.344E−04 | 2.11 | 91.5 |
| 448442 | AB033281 | Hs. 21229 | f-box and WD-40 domain protein 1B | 6.107E−03 | 2.11 | 52 |
| 429286 | AA449239 | Hs. 154855 | ESTs | 4.644E−04 | 2.11 | 35 |
| 439656 | AW138241 | Hs. 210846 | ESTs | 8.798E−05 | 2.11 | 45.25 |
| 438135 | AI253025 | Hs. 190426 | ESTs | 1.085E−03 | 2.11 | 99 |
| 407795 | AA044754 | Hs. 48821 | ESTs | 3.678E−04 | 2.11 | 58.5 |
| 447749 | T53260 | Hs. 191091 | ESTs | 7.069E−05 | 2.11 | 51.25 |
| 426685 | R20212 | Hs. 28454 | ESTs | 3.077E−03 | 2.10 | 40.25 |
| 440495 | AA887212 | Hs. 14161 | hypothetical protein DKFZp434I1930 | 5.008E−03 | 2.10 | 32.5 |
| 456373 | BE247706 | Hs. 89751 | membrane-spanning 4-domains, subfamily A | 2.022E−04 | 2.10 | 61.75 |
| 445817 | NM_003642 | Hs. 13340 | histone acetyltransferase 1 | 9.044E−04 | 2.10 | 52.75 |
| 449901 | AI674072 | | gb: wd15h01.x1 Soares_NFL_T_GBC_S1 Homo s | 5.149E−04 | 2.10 | 41.5 |
| 439161 | Y15164 | Hs. 6483 | oral-facial-digital syndrome 1 | 6.522E−04 | 2.10 | 38.75 |
| 442229 | AI885776 | Hs. 8164 | Mulibrey nanism | 7.120E−03 | 2.10 | 37.5 |
| 441024 | AW081530 | Hs. 268231 | ESTs | 2.934E−03 | 2.10 | 31.5 |
| 417691 | AU076610 | Hs. 82399 | low density lipoprotein receptor defect | 6.847E−03 | 2.10 | 30 |
| 445447 | H14522 | Hs. 8358 | ribosomal protein S4, X-linked | 2.286E−03 | 2.10 | 50 |
| 423703 | NM_014913 | Hs. 131915 | KIAA0863 protein | 1.086E−04 | 2.10 | 54 |
| 448749 | AW859679 | Hs. 21902 | Homo sapiens clone 25237 mRNA sequence | 2.491E−02 | 2.09 | 38 |
| 426272 | AW450671 | Hs. 189284 | ESTs | 2.953E−05 | 2.09 | 94 |
| 450124 | N95443 | Hs. 19180 | Homo sapiens mRNA; cDNA DKFZp564E122 (fr | 2.013E−03 | 2.09 | 47 |
| 413856 | D13639 | Hs. 75586 | cyclin D2 | 2.602E−03 | 2.09 | 45.75 |
| 446103 | U90918 | Hs. 13804 | hypothetical protein dJ462O23.2 | 1.900E−02 | 2.09 | 31 |
| 453173 | AB007902 | Hs. 32168 | KIAA0442 protein | 3.965E−06 | 2.09 | 169.25 |
| 450903 | AA011670 | Hs. 114749 | ESTs | 3.668E−04 | 2.09 | 40.75 |
| 421766 | AA481157 | Hs. 108110 | DKFZP547E2110 protein | 1.620E−03 | 2.08 | 43.25 |
| 409259 | AW608930 | Hs. 52184 | hypothetical protein FLJ20618 | 7.498E−07 | 2.08 | 83.5 |
| 438154 | AI671957 | Hs. 19523 | Homo sapiens clone 022f05 My030 protein | 1.646E−06 | 2.08 | 84.5 |
| 437469 | AW753112 | Hs. 15514 | hypothetical protein MGC3260 | 2.387E−03 | 2.08 | 55 |
| 451334 | AI122691 | Hs. 13268 | ESTs | 2.732E−04 | 2.08 | 49.25 |
| 418459 | R85436 | Hs. 268814 | ESTs | 2.866E−04 | 2.07 | 61.25 |
| 424737 | BE301883 | Hs. 152707 | glioblastoma amplified sequence | 1.146E−04 | 2.07 | 50.75 |
| 454038 | X06374 | Hs. 37040 | platelet-derived growth factor alpha pol | 2.713E−03 | 2.07 | 41.75 |
| 408951 | AW407227 | Hs. 49282 | hypothetical protein FLJ11088 | 3.336E−02 | 2.07 | 32.75 |
| 431923 | AI741770 | Hs. 292690 | ESTs, Weakly similar to I38022 hypotheti | 2.599E−03 | 2.07 | 43.5 |
| 447560 | AF065214 | Hs. 18858 | phospholipase A2, group IVC (cytosolic, | 4.846E−02 | 2.07 | 40.25 |
| 413836 | W92003 | Hs. 70614 | ESTs | 2.751E−02 | 2.07 | 30 |
| 450331 | AA009536 | Hs. 38323 | ESTs | 8.945E−03 | 2.07 | 34.75 |
| 446229 | AI744964 | Hs. 14449 | KIAA1609 protein | 9.450E−04 | 2.07 | 38.75 |
| 440706 | AA927562 | Hs. 148234 | ESTs | 2.101E−03 | 2.07 | 38.75 |
| 441872 | BE567100 | Hs. 154938 | hypothetical protein MDS025 | 6.092E−06 | 2.07 | 121.75 |
| 415710 | J04543 | Hs. 78637 | annexin A7 | 1.245E−02 | 2.07 | 31.75 |
| 428220 | BE183533 | Hs. 347128 | Human DNA sequence from clone 34B21 on c | 2.733E−06 | 2.07 | 76.5 |
| 438363 | AI886351 | Hs. 22353 | hypothetical protein FLJ21952 | 2.129E−04 | 2.07 | 65.25 |
| 414781 | D50917 | Hs. 77293 | KIAA0127 gene product | 2.237E−04 | 2.06 | 41 |
| 421297 | AB037360 | | KIAA1255 protein | 2.940E−05 | 2.06 | 74.5 |
| 452068 | W76412 | Hs. 57877 | ESTs | 9.454E−04 | 2.06 | 59 |
| 429788 | U87791 | Hs. 221040 | HBS1 (S. cerevisiae)-like | 6.130E−02 | 2.05 | 38.5 |
| 414718 | H95348 | Hs. 107987 | ESTs | 3.234E−02 | 2.05 | 53 |
| 421727 | Y13153 | Hs. 107318 | kynurenine 3-monooxygenase (kynurenine 3 | 1.389E−02 | 2.05 | 102.25 |
| 432401 | NM_013330 | Hs. 274479 | NME7 | 2.193E−03 | 2.05 | 104.5 |
| 448920 | AW408009 | Hs. 22580 | alkylglycerone phosphate synthase | 2.599E−03 | 2.05 | 53.25 |
| 433735 | AA608955 | Hs. 109653 | ESTs | 3.337E−03 | 2.05 | 41.25 |
| 415083 | AI632683 | Hs. 27179 | Homo sapiens cDNA FLJ12933 fis, clone NT | 2.842E−02 | 2.05 | 38.25 |
| 453078 | AF053551 | Hs. 31584 | metaxin 2 | 3.467E−03 | 2.05 | 37.5 |
| 453574 | AI767947 | Hs. 50841 | ESTs | 1.246E−02 | 2.05 | 34.25 |
| 426124 | AI268389 | Hs. 250697 | phosphatidylinositol glycan, class F | 4.291E−02 | 2.05 | 31.5 |
| 451408 | AI795947 | Hs. 209596 | ESTs | 1.438E−02 | 2.05 | 30 |
| 414553 | AI813865 | Hs. 164478 | hypothetical protein FLJ21939 similar to | 2.038E−02 | 2.05 | 39.25 |
| 431192 | AI670056 | Hs. 137274 | ESTs, Weakly similar to SP62_HUMAN SPLIC | 7.401E−03 | 2.04 | 53.25 |
| 457830 | BE147896 | Hs. 14662 | ESTs | 7.394E−03 | 2.04 | 31.5 |
| 449480 | AI741617 | Hs. 108447 | spinocerebellar ataxia 7 (olivopontocere | 4.046E−04 | 2.04 | 44.25 |
| 448384 | R12314 | Hs. 21056 | Homo sapiens cDNA: FLJ21366 fis, clone C | 1.085E−03 | 2.04 | 57.75 |
| 451798 | BE297567 | Hs. 27047 | hypothetical protein FLJ20392 | 1.480E−03 | 2.04 | 32.25 |
| 409245 | AA361037 | Hs. 288036 | tRNA isopentenylpyrophosphate transferas | 4.991E−02 | 2.04 | 39.25 |
| 446946 | AI878932 | Hs. 317 | topoisomerase (DNA) I | 2.490E−02 | 2.04 | 32.75 |
| 421166 | AA305407 | Hs. 102308 | potassium inwardly-rectifying channel, s | 1.298E−03 | 2.04 | 69.25 |
| 434275 | H92087 | Hs. 114288 | ESTs, Weakly similar to A47582 B-cell gr | 5.672E−04 | 2.04 | 127.5 |
| 428706 | AA432030 | Hs. 265827 | interferon, alpha-inducible protein (clo | 8.647E−07 | 2.04 | 242 |
| 435841 | R28522 | Hs. 186937 | ESTs | 3.666E−04 | 2.03 | 44.5 |
| 422684 | BE561617 | Hs. 119192 | H2A histone family, member Z | 2.714E−03 | 2.03 | 53.25 |
| 410333 | AL049538 | Hs. 62349 | ras association (RalGDS/AF-6) domain con | 1.157E−02 | 2.03 | 33.25 |
| 446126 | AW085909 | Hs. 10177 | pleckstrin homology domain interacting p | 3.205E−03 | 2.03 | 62 |
| 421628 | AL121317 | Hs. 106210 | hypothetical protein FLJ10813 | 3.780E−03 | 2.03 | 56.75 |
| 448939 | BE267795 | Hs. 22595 | hypothetical protein FLJ10637 | 1.246E−02 | 2.03 | 49.25 |
| 420747 | BE294407 | Hs. 99910 | phosphofructokinase, platelet | 6.596E−03 | 2.03 | 50.25 |
| 420977 | AW794019 | Hs. 100651 | golgi SNAP receptor complex member 2 | 4.088E−03 | 2.03 | 30.5 |
| 432886 | BE159028 | Hs. 279704 | chromatin accessibility complex 1 | 4.034E−02 | 2.02 | 52 |
| 432960 | AW150945 | Hs. 144758 | ESTs | 8.629E−04 | 2.02 | 45.25 |
| 429857 | AF089897 | Hs. 294030 | topoisomerase-related function protein 4 | 4.393E−06 | 2.02 | 43.75 |
| 407879 | AA045464 | Hs. 6557 | zinc finger protein 161 | 3.776E−03 | 2.02 | 39.75 |
| 428708 | NM_014897 | Hs. 190386 | KIAA0924 protein | 1.543E−02 | 2.02 | 36.75 |
| 424670 | W61215 | Hs. 116651 | epithelial V-like antigen 1 | 1.091E−04 | 2.02 | 93 |
| 421315 | D78791 | Hs. 103419 | fasciculation and elongation protein zet | 6.077E−06 | 2.02 | 90.25 |
| 437370 | AL359567 | Hs. 161962 | Homo sapiens mRNA; cDNA DKFZp547D023 (fr | 3.501E−04 | 2.02 | 48.75 |
| 445330 | R52656 | Hs. 21691 | ESTs | 8.939E−03 | 2.02 | 63.75 |
| 433882 | U90441 | Hs. 3622 | procollagen-proline, 2-oxoglutarate 4-di | 7.522E−04 | 2.02 | 57.5 |
| 425409 | H18021 | Hs. 25005 | hypothetical protein MGC3329 | 1.620E−03 | 2.02 | 30.75 |
| 407807 | AL031427 | Hs. 40094 | Human DNA sequence from clone 167A19 on | 6.339E−03 | 2.01 | 34 |
| 414900 | AW452420 | Hs. 248678 | ESTs | 3.481E−03 | 2.01 | 57.25 |
| 408194 | AA601038 | Hs. 191797 | ESTs, Weakly similar to S65657 alpha-1C- | 2.035E−02 | 2.01 | 38.75 |
| 410582 | AW867197 | Hs. 337561 | hypothetical protein FLJ21616 | 5.072E−06 | 2.01 | 103.5 |
| 422576 | BE548555 | Hs. 118554 | CGI-83 protein | 6.601E−03 | 2.01 | 79.5 |
| 418504 | BE159718 | Hs. 85335 | Homo sapiens mRNA; cDNA DKFZp564D1462 (f | 3.692E−05 | 2.01 | 80 |
| 422431 | AI769410 | Hs. 221461 | ESTs | 3.471E−03 | 2.01 | 34.5 |
| 426369 | AF134157 | Hs. 169487 | Kreisler (mouse) maf-related leucine zip | 1.765E−05 | 2.01 | 123.75 |
| 413949 | AA316077 | Hs. 75639 | Human TB1 gene mRNA, 3′ end | 2.410E−02 | 2.01 | 42.5 |
| 451625 | R56793 | Hs. 106576 | alanine-glyoxylate aminotransferase 2-li | 2.789E−05 | 2.00 | 184.5 |
| 405688 | | | NM_018850*: Homo sapiens ATP-binding cass | 1.551E−03 | 2.00 | 83.25 |
| 418838 | AW385224 | Hs. 35198 | ectonucleotide pyrophosphatase/phosphodi | 7.726E−06 | 2.00 | 64.5 |
| 437672 | AW748265 | Hs. 5741 | flavohemoprotein b5? | 6.821E−04 | 2.00 | 45.75 |
| 458389 | H70284 | Hs. 160152 | ESTs, Weakly similar to FPHU alpha-fetop | 8.798E−05 | 2.00 | 45.25 |
| 417727 | AL133623 | Hs. 82501 | similar to mouse Xrn1/Dhm2 protein | 6.585E−03 | 2.00 | 41 |
| 444743 | AA045648 | Hs. 301957 | nudix (nucleoside diphosphate linked moi | 1.715E−02 | 2.00 | 40.75 |
| 429167 | BE465867 | Hs. 197751 | KIAA0666 protein | 7.118E−03 | 2.00 | 40.25 |
| 451141 | AW772713 | Hs. 247186 | ESTs | 2.599E−03 | 2.00 | 40 |
| 430178 | AW449612 | Hs. 152475 | ESTs | 1.337E−02 | 2.00 | 34.75 |
| 421620 | AA446183 | Hs. 91885 | ESTs, Weakly similar to I55214 salivary | 4.811E−03 | 2.00 | 33.25 |
| 439898 | AW505514 | Hs. 209561 | KIAA1715 protein | 2.596E−03 | 2.00 | 32.5 |
| 452874 | AK001061 | Hs. 30925 | hypothetical protein FLJ10199 | 3.337E−02 | 2.00 | 30 |

Pkey: Unique Eos probeset identifier number
ExAccn: Exemplar Accession number, Genbank accession number
UnigeneID: Unigene number
Unigene Title: Unigene gene title
R1: Wilcoxon rank-sum test p-value
R2: Trimean of Hep C+ Liver over Trimean of Hep C− liver Ratio
R3: Difference of Trimean of Hep C+ Liver vs. Trimean of Hep C− Liver

TABLE 3B

| Pkey | CAT Number | Accession |
|---|---|---|
| 407992 | 103172_2 | AW418811 AI743200 AI458141 |
| 408731 | 10774_1 | R85652 AA114024 AA296219 AA375304 AW963796 AW885952 AW020969 AA114025 AI804930 BE350971 AI765355 |
| | | AW317067 AW974763 |
| | | H85930 AW172600 AI310231 AW612019 D62908 D62864 AA652738 AI674617 AI494064 AW138666 AI147620 |
| | | AI147629 AW611793 AI668922 |
| | | AI971005 AI864742 AA174171 |
| 410099 | 117647_1 | AA081630 T08671 AI174254 D83874 AW959843 AA364503 AA693467 AW993370 BE327037 AA167714 |
| | | N79906 AW901977 AW901980 W52882 |
| | | T07735 AA484549 W60090 D52685 T23811 BE327043 AW901768 BE551237 AA917004 AA716027 AI439658 |
| | | AA283724 AI805992 AI457096 |
| | | AA084618 BE467736 AI092635 AI887863 AI697593 AA436618 AI167419 AI418634 T31586 AA436630 AA706191 |
| | | AI041169 AI422304 T03534 |
| | | AA211402 AI204899 AI366472 AW827081 AA788593 T32736 AI767935 AA167791 AA747914 AA663870 AI865504 |
| 411590 | 125064_1 | T96183 T64070 AA094134 |
| 411656 | 1252973_1 | AW855576 AW855650 AW855578 AW855577 AW855642 AW855619 AW855624 AW855621 |
| 411777 | 1257741_1 | BE067552 BE067800 BE067593 BE067714 BE067819 H49900 |
| 413007 | 1343540_1 | BE046662 BE046697 BE046655 |
| 413509 | 1374313_1 | BE145419 BE145433 |
| 413645 | 138145_1 | AA130992 AA503835 AW969537 |
| 414279 | 143227_1 | AW021691 AI537404 R45431 AI333439 AI741845 AI674468 R44190 R52535 R52617 AI220925 AI979148 AI744688 |
| | | AW242437 AA618148 |
| | | AI983837 AA399623 AI676204 AI420077 N24944 D51042 AA282786 AA137264 AW236107 AW769997 N53529 |
| | | AI624731 AI968243 AI863637 |
| | | Z41183 N38931 H99461 AI129279 AI767302 AI474904 H72893 AI434776 AI498342 AI089287 AA398984 H79114 |
| | | N33821 AA625451 AA282874 |
| | | AA137263 H72493 AF236691 AA482849 R23405 R25093 R53271 BE073129 R52586 BE073137 Z33587 AW080738 |
| | | Z45484 AW889665 |
| 414496 | 145392_1 | W73853 AA928112 W77887 AW889237 AA148524 AI749182 AI754442 AI338392 AI253102 AI079403 AI370541 |
| | | AI697341 H97538 AW188021 |
| | | AI927669 W72716 AI051402 AI188071 AI335900 N21488 AW770478 W92522 AI691028 AI913512 AI144448 W73819 |
| | | AA604358 N28900 W95221 |
| | | AI868132 H98465 AA148793 |
| 414588 | 146467_1 | AA302905 AA495793 AA149783 |
| 416475 | 1596398_1 | T70298 H58072 R02750 |
| 416611 | 160379_1 | AA568308 AI453629 AI984473 AI341559 AI983169 AA181902 AI708321 AI695791 AI695792 AI478300 |
| 418259 | 173388_1 | AA215404 AI990909 BE464132 AW271459 N74332 AI262061 |
| 418304 | 173658_2 | AA215702 AA368006 AA215703 BE066555 BE006876 |
| 418876 | 179960_1 | AA740616 AA654854 AA229923 |
| 420825 | 196769_1 | AI656727 AI697887 AI802122 AA910877 Z28718 T16711 AA651731 AL047264 BE000621 R68736 AW992695 |
| | | AI768764 AW271284 AW974653 |
| | | AI308951 AW055146 R93609 AW467031 AI096866 AI371871 AI126182 AI564756 AI361460 AI358914 AI419231 |
| | | AW439733 R87059 AA628064 |
| | | AW088970 AW008695 R68682 AI719136 R97752 AW196262 |
| 420985 | 19829_1 | X94703 NM_004249 R52316 T87420 N46403 Z36855 BE076834 |
| 421057 | 198849_1 | T58283 AA765038 AA283052 H99396 AA814751 AI032674 N81016 N81017 BE222349 AA830545 |
| 421272 | 200810_1 | AA704157 AA286825 C15898 C15173 D81736 AI810506 BE466071 H84948 R88291 H85515 AW014470 |
| 421297 | 200_1 | AB037360 NM_016376 AB033081 T25038 R94364 AA399607 AW814619 BE172335 AW609548 T83895 N40773 |
| | | T86064 N40777 N40757 |
| | | BE550945 AW450283 H62663 AI298087 AI288098 N55959 T83725 H65293 N30050 AW858563 AW858561 |
| | | AW858575 AW861931 AW858569 |
| | | AW363633 N30072 AW858524 AW754343 AW754342 AW604731 AW754345 AI363734 N30068 BE155949 T16974 |
| | | R60281 BE071291 AI423079 |
| | | BE071350 AI917153 AA256914 AA436381 AW383355 AI630341 AW383736 AW388181 BE002858 |
| | | AW815513 AA351367 |
| 421718 | 20595_1 | AL117574 AI681945 BE503055 AW015330 C16652 AA441855 AA329853 |
| | | AW962502 AA441830 AF114049 |
| 423886 | 232905_1 | AA332098 AW950499 AW950497 |
| 423983 | 233891_1 | AA333261 AA333365 AA552870 |
| 425303 | 249780_1 | AA354785 AA354792 AW958427 |
| 425757 | 255956_1 | AA363171 AW963347 AA371863 |
| 427213 | 276052_1 | AW007211 AA399252 AA960991 BE467259 |
| 427384 | 278072_1 | T82854 AA401778 R92652 |
| 428342 | 290035_2 | AI739168 AA426249 AI199636 AW505198 AW977291 AA824583 AA883419 AA724079 AI015524 AI377728 AW293682 |
| | | AI928140 AA731438 |
| | | AI092404 AI085630 AA731340 |
| 429978 | 31150_1 | AA249027 AL038984 AK001993 AL080066 AV652725 BE566226 AA345557 AA315222 AA090585 AA375688 |
| | | AA301092 AA298454 W05762 |
| | | AW607939 H51658 D83880 N84323 BE296821 AW947007 D61461 AW079261 AA329482 AW901780 AI354442 |
| | | AA772275 R31663 AI354441 |
| | | AI767525 H92431 AI916735 H93575 AI394255 AW014741 AI573090 C06195 AW612857 AW265195 AI339558 |
| | | AI377532 AI308821 AI919424 |
| | | AI589705 AW055215 AI336532 AI338051 AA806547 C75509 C00618 AW071172 AW769904 AA630381 AI678018 |
| | | AI863985 D79662 BE221049 |
| | | AW265018 AI589700 AW196655 N76573 AI370908 BE042393 N75017 AI698870 AW960115 |
| 430068 | 312849_1 | AA464964 M85405 AA947566 |
| 432474 | 348197_1 | AA584042 AW973273 AA548798 |
| 432600 | 350959_1 | AI821085 AW973464 AA554802 AI821831 AA657438 AA640756 AA650339 |
| 432676 | 352582_2 | AI187366 AA558869 AA618478 |
| 432689 | 35275_1 | AB018320 H56457 AA247916 N83488 N87920 AA095653 T19858 AL134279 AA094167 AI673378 AI000340 |
| | | R47500 W16595 AW152297 AI625937 |
| | | AA002027 AI814851 AA902666 AI039729 AW975053 BE302243 AI240793 AA193203 N55818 AI886651 |
| | | AA877735 AA565288 AI284399 |
| | | AA707069 AA775885 AI014967 AA524249 R56212 AA878627 H80252 AA085731 AA657859 AI753853 AA564328 |
| | | H83107 H56458 AA193204 |
| | | AI075230 AA001823 AI269462 F09349 N87900 AA653028 T71600 T71751 T71812 R58128 AI890218 AL134289 |
| | | T71659 AA323827 N92477 |
| | | T54867 AA985252 F11689 R56295 BE086764 N88917 AA013161 BE407758 AW993041 BE018672 AL120628 |
| | | H54590 AW364176 |
| 432709 | 353139_1 | H17238 AA563739 F05559 F06116 |
| 433464 | 367082_1 | N92481 AI475594 AW674508 AA593748 AW974058 |
| 433847 | 374914_1 | AA610266 AA610273 AA632625 AA812563 AI688018 AI094802 H79160 R99139 |
| 433891 | 376239_1 | AA613792 AW182329 T05304 AW858385 |
| 434280 | 382816_1 | BE005398 AA628622 AA994155 |
| 437151 | 433855_1 | AA745618 AA745616 AW805449 BE159081 |
| 438141 | 450685_1 | AW946871 AW946782 AW946955 AA778849 |
| 438980 | 467544_1 | AW502384 AI982587 AA828822 |
| 439046 | 468133_1 | AA947354 AA829660 AI687296 |
| 439195 | 46967_1 | H89360 AF086037 H89546 |
| 440638 | 499025_1 | AI376551 T87714 AA897445 |
| 442048 | 531432_1 | AA974603 AI984319 AW340495 |
| 442092 | 532588_1 | AW578669 AI862106 AW025563 AW193658 AA976004 |
| 444314 | 600667_1 | AI140497 AW749625 AW749626 AW749644 |
| 448219 | 75529_4 | AA228092 AW577775 AI033234 BE076308 AI377195 AW577769 |
| 449001 | 792914_1 | AI619957 AI654836 R99473 H51659 R99472 |
| 449901 | 818599_1 | AI674072 BE268487 |
| 451107 | 85931_1 | AA235108 W00590 H30674 T12414 AA384068 AA482520 AA325367 AW955325 AW517234 AI749193 |
| | | AW500012 AW503974 AI720354 AI769223 |
| | | AW058126 AI371974 AW021415 AA017421 AI027749 R55450 W80453 AA601599 N63625 AA235217 |
| | | AA015723 AI492264 AI075880 AW444749 |
| | | AI056860 AI684298 AI826258 AA055649 AA558483 AAI59479 AA533567 AA496743 AA417823 AA069759 |
| | | AA057021 BE247112 N68913 |
| | | AA486633 AA486538 N93443 T12413 |
| 452480 | 919099_1 | AI903526 AI903572 AI903574 AI903571 |
| 453929 | 98823_1 | AW190054 AI263834 AA040074 |
| 456034 | 142696_1 | AW450979 AA136653 AA136656 AW419381 AA984358 AA492073 BE168945 AA809054 AW238038 |
| | | BE011212 BE011359 BE011367 BE011368 |
| | | BE011362 BE011215 BE011365 BE011363 |
| 456721 | 222858_1 | AA533356 AW468427 R67736 AA779031 AA614088 AI823404 AA318991 AA720986 |
| 457247 | 308656_1 | AA458605 AW977252 AI261627 AW274550 AI418272 AW665579 AA731376 AW293861 D80453 AI217860 |

Pkey: Unique Eos probeset identifier number
CAT number: Gene cluster number
Accession: Genbank accession numbers

TABLE 3C

| Pkey | Ref | Strand | Nt_position |
|---|---|---|---|
| 400517 | 9796686 | Minus | 49996-50346 |
| 401016 | 8117441 | Plus | 126234-126359, 128050-128236 |
| 401091 | 9958240 | Plus | 94760-94898 |
| 401197 | 9719705 | Plus | 176341-176452 |
| 401403 | 7710966 | Plus | 146180-146294 |
| 401898 | 8570008 | Minus | 72013-72132, 72400-72487 |
| 401928 | 3873182 | Plus | 54932-55070 |
| 401952 | 3319121 | Minus | 53770-53979 |
| 402041 | 7770639 | Plus | 69353-69454 |
| 402439 | 9796503 | Minus | 108604-108764 |
| 402507 | 9797889 | Plus | 118979-119086 |
| 402727 | 9211324 | Plus | 54596-54777 |
| 402737 | 9212184 | Minus | 13358-13552 |
| 402847 | 9408716 | Minus | 104107-104314 |
| 402964 | 9581599 | Minus | 46624-46784 |
| 403027 | 7670575 | Plus | 60696-60932, 61362-61521 |
| 403330 | 8516153 | Plus | 116558-116698 |
| 403575 | 8101156 | Minus | 81961-82068 |
| 403576 | 6862645 | Minus | 73475-73940, 74079-74207 |
| 403707 | 7108128 | Minus | 132794-133294 |
| 403738 | 7212067 | Plus | 38434-38562, 56876-57007, |
| | | | 59789-59876, 60714-60933, |
| | | | 62986-63265 |
| 403743 | 7652003 | Minus | 136463-136646 |
| 403790 | 8084957 | Minus | 87826-87947, 89835-90002 |
| 403809 | 8568861 | Plus | 33910-34129, 34583-34862 |
| 403976 | 7657840 | Plus | 24755-24969 |
| 404149 | 7534008 | Plus | 121831-121951, 124044-124150 |
| 404240 | 5002624 | Minus | 116132-116407, 116653-116922 |
| 404298 | 9944263 | Minus | 73591-73723 |
| 404676 | 9797204 | Minus | 56167-56342, 58066-58189, |
| | | | 58891-59048, 60452-60628 |
| 405102 | 8076881 | Minus | 120922-121296 |
| 405141 | 8980911 | Plus | 99861-100054 |
| 405268 | 4156151 | Minus | 24404-24521 |
| 405270 | 4156145 | Minus | 3952-4123, 6886-7010, 8541-8728 |
| 405688 | 4508117 | Minus | 88702-88899 |
| 405689 | 4508117 | Minus | 92558-92698, 94282-94382, |
| | | | 97977-98180, 99202-99285 |
| 406038 | 8389537 | Plus | 37764-37877 |
| 406274 | 7543787 | Plus | 932-1123 |
| 406423 | 9256411 | Plus | 165600-165824 |

Pkey: Unique number corresponding to an Eos probeset
Ref: Sequence source. The 7 digit numbers in this column are Genbank Identifier (GI) numbers. “Dunham, et al” refers to the publication entitled “The DNA sequence of human chromosome 22” Dunham, et al. (1999) Nature 402: 489-495.
Strand: Indicates DNA strand from which exons were predicted.
Nt_position: Indicates nucleotide positions of predicted exons.

Table 4A lists about 200 genes down-regulated in Hepatitis C positive liver tissues compared to Hepatitis C negative liver tissues. These were selected from 59680 probesets on the Affymetrix/Eos Hu03 GeneChip array such that the Wilcoxon rank-sum test p-value between the 2 groups was less than 0.10, the ratio of the “weighted average” of Hepatitis C negative liver tissues to the “weighted average” of Hepatitis C positive liver tissues was equal to or above 2.0, and that the differences between the same 2 groups was equal to or above 30.0. The “weighted average” of the Hepatitis C negative liver tissues was set to the trimean of various different Hepatitis C negative liver tissues. The “weighted average” of the Hepatitis C positive liver tissues was set to the either 10 or the trimean of various different Hepatitis C positive liver tissues, whichever value was greater to eliminate ratios with a denominator of zero or less. Tables 4B and 4C relate to Table 4A as 3B and 3C relate to 3A.

TABLE 4A
ABOUT 200 GENES DOWN-REGULATED IN HEPATITIS C POSITIVE LIVER TISSUES COMPARED TO
HEPATITIS C NEGATIVE LIVER TISSUES

| Pkey | ExAccn | UnigeneID | Unigene Title | R1 | R2 | R3 |
|---|---|---|---|---|---|---|
| 450912 | AW939251 | Hs. 25647 | v-fos FBJ murine osteosarcoma viral onco | 2.125E−06 | 21.50 | 645.75 |
| 451831 | NM_001674 | Hs. 460 | activating transcription factor 3 | 1.410E−04 | 19.50 | 189.75 |
| 414559 | AV656184 | Hs. 76452 | C-reactive protein, pentraxin-related | 1.209E−04 | 19.31 | 425.75 |
| 447078 | AW885727 | Hs. 9914 | ESTs | 2.578E−02 | 14.00 | 138 |
| 405278 | | | NM_002864: Homo sapiens pregnancy-zone pr | 1.132E−03 | 11.92 | 115.5 |
| 451029 | AA852097 | Hs. 25829 | ras-related protein | 7.883E−04 | 11.12 | 346.75 |
| 416188 | BE157260 | Hs. 79070 | v-myc avian myelocytomatosis viral oncog | 7.173E−04 | 9.65 | 103.5 |
| 442941 | AU076728 | Hs. 8867 | cysteine-rich, angiogenic inducer, 61 | 1.830E−04 | 8.74 | 309.5 |
| 434078 | AW880709 | Hs. 283683 | chromosome 8 open reading frame 4 | 1.545E−02 | 8.47 | 76.75 |
| 414220 | BE298094 | Hs. 323806 | gb: 601118231F1 NIH_MGC_17 Homo sapiens c | 3.560E−02 | 6.63 | 57.25 |
| 448607 | AL042506 | Hs. 21599 | Kruppel-like factor 7 (ubiquitous) | 2.189E−03 | 6.50 | 57.5 |
| 446066 | AI343931 | Hs. 149383 | ESTs | 3.476E−03 | 6.42 | 54.75 |
| 404501 | AW247252 | | nucleoside phosphorylase | 2.939E−02 | 5.93 | 53 |
| 420101 | AW500529 | Hs. 95180 | KIAA0767 protein | 1.621E−03 | 5.84 | 61.75 |
| 407173 | T64349 | | gb: yc10d08.s1 Stratagene lung (937210) H | 1.543E−02 | 5.80 | 55 |
| 420548 | AA278246 | Hs. 920 | ESTs | 5.637E−03 | 5.46 | 45.75 |
| 414327 | BE408145 | Hs. 185254 | ESTs, Weakly similar to T24435 hypotheti | 5.401E−04 | 5.45 | 63.5 |
| 416434 | AW163045 | Hs. 79334 | nuclear factor, interleukin 3 regulated | 1.900E−02 | 5.45 | 101.75 |
| 427509 | M62505 | Hs. 2161 | complement component 5 receptor 1 (C5a I | 1.439E−02 | 5.43 | 89.75 |
| 419299 | AI311085 | Hs. 62406 | hypothetical protein FLJ22573 | 3.630E−03 | 5.05 | 45.25 |
| 400425 | AY004252 | Hs. 287385 | PR domain containing 12 | 1.922E−04 | 4.88 | 39.75 |
| 417752 | C15737 | Hs. 269386 | ESTs | 4.093E−03 | 4.80 | 43 |
| 430070 | AF197927 | Hs. 231967 | ALL1 fused gene from 5q31 | 4.351E−05 | 4.80 | 51.25 |
| 401149 | | | Target Exon | 1.567E−04 | 4.75 | 45.75 |
| 409233 | AK002001 | Hs. 51305 | v-maf musculoaponeurotic fibrosarcoma (a | 2.734E−04 | 4.74 | 80.5 |
| 423053 | BE312679 | | gb: 601148138F1 NIH_MGC_19 Homo sapiens c | 1.242E−03 | 4.72 | 64.25 |
| 453196 | AW003567 | Hs. 345309 | ESTs | 2.602E−03 | 4.65 | 41.75 |
| 408278 | AW876813 | Hs. 3343 | phosphoglycerate dehydrogenase | 2.866E−04 | 4.52 | 35.5 |
| 401677 | H18444 | | BAI1-associated protein 3 | 2.177E−02 | 4.40 | 34.5 |
| 428106 | BE620016 | Hs. 182470 | PTD010 protein | 8.236E−04 | 4.38 | 57.25 |
| 427557 | NM_002659 | Hs. 179657 | plasminogen activator, urokinase recepto | 8.798E−05 | 4.34 | 71 |
| 418303 | AA215701 | Hs. 186541 | ESTs, Weakly similar to I38022 hypotheti | 2.749E−02 | 4.32 | 44.75 |
| 431319 | AA873350 | Hs. 302232 | ESTs | 5.146E−02 | 4.20 | 81.75 |
| 449986 | AW864502 | | gb: PM4-SN0016-120400-004-b12 SN0016 Homo | 7.698E−02 | 4.17 | 33.75 |
| 458867 | AW995393 | | gb: QV0-BN0042-170300-163-g12 BN0042 Homo | 5.074E−07 | 4.16 | 126.25 |
| 423720 | AL044191 | Hs. 23388 | hypothetical protein DKFZp434F0318 | 5.213E−03 | 4.15 | 82.25 |
| 433071 | BE150229 | Hs. 281564 | retinal outer segment membrane protein 1 | 1.922E−03 | 4.15 | 32.25 |
| 450335 | BE218355 | Hs. 201781 | ESTs, Weakly similar to B34087 hypotheti | 1.000E−02 | 4.13 | 33.75 |
| 448871 | BE616709 | Hs. 159265 | kruppel-related zinc finger protein hcKr | 5.425E−03 | 4.10 | 47.25 |
| 409795 | AI934808 | Hs. 219132 | ESTs, Weakly similar to T46338 hypotheti | 3.624E−03 | 4.02 | 38.5 |
| 401913 | | | ENSP00000249158*: CDNA | 3.560E−02 | 4.00 | 33 |
| 403796 | | | Target Exon | 1.239E−03 | 3.97 | 44.25 |
| 436068 | AK000038 | Hs. 300979 | ESTs, Weakly similar to I38022 hypotheti | 1.038E−02 | 3.95 | 31.25 |
| 449236 | AJ403126 | Hs. 26373 | Homo sapiens cDNA: FLJ23449 fis, clone H | 2.255E−02 | 3.90 | 35.5 |
| 431394 | AK000692 | Hs. 252351 | HERV-H LTR-associating 2 | 3.334E−03 | 3.88 | 35.25 |
| 454985 | AW849292 | | gb: IL3-CT0215-020300-090-E06 CT0215 Homo | 2.711E−03 | 3.88 | 42 |
| 444531 | BE158822 | Hs. 282469 | ESTs, Weakly similar to I38022 hypotheti | 4.629E−03 | 3.85 | 30.5 |
| 441063 | AA913819 | Hs. 188025 | ESTs | 9.074E−02 | 3.77 | 33.5 |
| 431861 | AA521072 | Hs. 292128 | ESTs | 3.666E−04 | 3.76 | 31.75 |
| 402517 | | | Target Exon | 1.902E−02 | 3.76 | 33.75 |
| 454598 | AW809716 | | gb: MR4-ST0124-241199-026-h09 ST0124 Homo | 7.123E−03 | 3.75 | 36 |
| 415477 | NM_002228 | Hs. 78465 | v-jun avian sarcoma virus 17 oncogene ho | 3.862E−04 | 3.75 | 135.25 |
| 411349 | AW838313 | | gb: QV2-LT0051-030500-188-f06 LT0051 Homo | 7.860E−04 | 3.70 | 35 |
| 410267 | AW978005 | Hs. 12600 | N-ethylmaleimide-sensitive factor attach | 2.814E−03 | 3.65 | 32.25 |
| 454292 | N57559 | Hs. 82273 | hypothetical protein | 7.123E−03 | 3.65 | 53 |
| 404958 | | | Target Exon | 2.491E−03 | 3.63 | 37.25 |
| 403794 | | | Target Exon | 1.763E−03 | 3.61 | 45 |
| 412176 | AW898334 | | gb: RC3-NN0070-270400-011-f02 NN0070 Homo | 7.511E−04 | 3.60 | 30 |
| 445402 | AI222415 | Hs. 147852 | ESTs | 6.522E−04 | 3.60 | 34.5 |
| 439760 | AL355741 | Hs. 21641 | Homo sapiens mRNA full length insert cDN | 7.856E−04 | 3.55 | 37.5 |
| 450875 | AK000724 | Hs. 301553 | karyopherin alpha 6 (importin alpha 7) | 2.938E−05 | 3.52 | 83.25 |
| 417551 | AI816291 | Hs. 82273 | hypothetical protein | 8.224E−04 | 3.52 | 103.25 |
| 429258 | AA448765 | | gb: zx10e09.r1 Soares_total_fetus_Nb2HF8_{—} | 5.001E−03 | 3.51 | 33.25 |
| 403324 | | | C2000428*: gi\|7705383\|ref\|NP_057536.1\|GC | 2.038E−02 | 3.50 | 30 |
| 411929 | AA098880 | Hs. 69297 | ESTs | 9.630E−03 | 3.50 | 50 |
| 418525 | AW450369 | Hs. 86937 | ESTs | 1.199E−02 | 3.47 | 32.5 |
| 403579 | | | Target Exon | 1.158E−02 | 3.47 | 38.25 |
| 449335 | AW150717 | Hs. 345728 | STAT induced STAT inhibitor 3 | 4.096E−03 | 3.47 | 221.25 |
| 414428 | BE296906 | Hs. 182625 | VAMP (vesicle-associated membrane protei | 5.786E−02 | 3.45 | 43.5 |
| 449785 | AI225235 | Hs. 288300 | hypothetical protein FLJ23231 | 2.255E−02 | 3.45 | 35.25 |
| 430389 | AL117429 | Hs. 240845 | DKFZP434D146 protein | 1.489E−02 | 3.42 | 31.5 |
| 454636 | AW811502 | | gb: QV2-ST0145-061299-015-b04 ST0145 Homo | 2.830E−03 | 3.42 | 38.75 |
| 402889 | | | ENSP00000217123*: FLJ00118 protein (Fragm | 2.011E−03 | 3.42 | 36.25 |
| 405600 | | | C12001673: gi\|9631264\|ref\|NP_048045.1\|or | 4.808E−03 | 3.39 | 36.5 |
| 408120 | AW299900 | Hs. 267632 | TATA element modulatory factor 1 | 5.865E−03 | 3.38 | 37.75 |
| 446052 | AA358760 | | gb: EST67699 Fetal lung II Homo sapiens c | 3.886E−05 | 3.36 | 72 |
| 409840 | AW502122 | | gb: UI-HF-BR0p-ajr-c-08-0-Ul.r1 NIH_MGC_5 | 9.284E−05 | 3.36 | 39.5 |
| 415897 | H08323 | Hs. 268712 | ESTs | 7.148E−04 | 3.35 | 30 |
| 456782 | AK000462 | Hs. 132071 | ovarian carcinoma immunoreactive antigen | 1.409E−04 | 3.35 | 38.75 |
| 417732 | R36065 | | gb: yg69h06.r1 Soares infant brain 1NIB H | 7.852E−04 | 3.31 | 31.25 |
| 402273 | | | Target Exon | 2.933E−02 | 3.30 | 34 |
| 440535 | AI590563 | Hs. 125910 | ESTs | 1.339E−02 | 3.30 | 32.75 |
| 450799 | AW407504 | | gb: UI-HF-BM0-adk-g-12-0-Ul.r1 NIH_MGC_38 | 1.768E−03 | 3.27 | 42.5 |
| 403508 | | | Target Exon | 2.106E−02 | 3.25 | 36 |
| 443725 | AW245680 | Hs. 9701 | growth arrest and DNA-damage-inducible, | 5.085E−06 | 3.24 | 412.5 |
| 427536 | BE277141 | Hs. 115803 | gb: 601178666F1 NIH_MGC_20 Homo sapiens c | 9.859E−04 | 3.20 | 38 |
| 447306 | AI373163 | Hs. 170333 | ESTs | 2.254E−02 | 3.17 | 39.5 |
| 430071 | AA355986 | Hs. 232068 | transcription factor 8 (represses interl | 1.243E−03 | 3.15 | 142.75 |
| 410052 | AA525225 | Hs. 334630 | Homo sapiens cDNA FLJ14462 fis, clone MA | 1.903E−02 | 3.11 | 107.75 |
| 408952 | S79854 | Hs. 49322 | deiodinase, iodothyronine, type III | 2.471E−04 | 3.09 | 35.5 |
| 447819 | U90544 | Hs. 19710 | solute carrier family 17 (sodium phospha | 4.558E−02 | 3.07 | 30.5 |
| 433010 | AW970018 | | gb: EST382097 MAGE resequences, MAGK Homo | 2.492E−03 | 3.05 | 36.5 |
| 442364 | AA993149 | Hs. 129895 | ESTs, Moderately similar to TBX3_HUMAN T | 2.193E−03 | 3.05 | 32.5 |
| 403582 | | | Target Exon | 5.956E−02 | 3.02 | 33.75 |
| 449180 | AI633836 | Hs. 195649 | ESTs | 5.785E−02 | 3.02 | 30.25 |
| 415994 | NM_002923 | Hs. 78944 | regulator of G-protein signalling 2, 24k | 5.146E−02 | 3.02 | 117 |
| 447135 | T58148 | | gb: yb98g06.s1 Stratagene lung (937210) H | 2.012E−03 | 3.00 | 62.5 |
| 419555 | AA244416 | | gb: nc07d11.s1 NCI_CGAP_Pr1 Homo sapiens | 2.196E−03 | 2.99 | 95.5 |
| 412568 | AI878826 | Hs. 74034 | caveolin 1, caveolae protein, 22 kD | 3.035E−02 | 2.98 | 61 |
| 444047 | AI097452 | Hs. 135095 | ESTs | 1.900E−02 | 2.97 | 35.5 |
| 403180 | | | Target Exon | 3.858E−04 | 2.96 | 38.75 |
| 402777 | | | C1002652*: gi\|544327\|sp\|Q04799\|FMO5_RABIT | 1.117E−02 | 2.94 | 41.25 |
| 433095 | AK001092 | Hs. 302480 | Homo sapiens cDNA FLJ10230 fis, clone HE | 1.902E−02 | 2.93 | 33.75 |
| 402070 | | | Target Exon | 7.109E−03 | 2.89 | 35.5 |
| 402124 | | | NM_031891: Homo sapiens cadherin 20, type | 8.621E−04 | 2.88 | 36.25 |
| 402663 | | | C1002133: gi\|12697931\|dbj\|BAB21784.1\| (AB | 8.917E−03 | 2.88 | 30.5 |
| 447721 | BE619620 | | lysyl oxidase | 1.845E−03 | 2.85 | 32.25 |
| 422017 | NM_003877 | Hs. 110776 | STAT induced STAT inhibitor-2 | 3.480E−03 | 2.84 | 108 |
| 432877 | AW974111 | Hs. 292477 | ESTs | 4.422E−02 | 2.84 | 67 |
| 444863 | AW384082 | Hs. 104879 | serine (or cysteine) proteinase inhibito | 2.331E−02 | 2.80 | 37.5 |
| 444207 | AI565004 | Hs. 343475 | cathepsin D (lysosomal aspartyl protease | 4.591E−05 | 2.80 | 194 |
| 428568 | AC004755 | Hs. 302038 | Homo sapiens chromosome 19, fosmid 37502 | 7.492E−04 | 2.80 | 46.25 |
| 424042 | Y10601 | Hs. 137674 | ankyrin-like with transmembrane domains | 7.392E−03 | 2.78 | 35.25 |
| 440509 | BE410132 | Hs. 134202 | ESTs, Weakly similar to T17279 hypotheti | 4.906E−04 | 2.78 | 94.75 |
| 459721 | AI299050 | Hs. 143835 | gb: qn14d12.x1 NCI_CGAP_Lu5 Homo sapiens | 4.287E−02 | 2.78 | 37.75 |
| 402651 | | | NM_000721*: Homo sapiens calcium channel, | 2.705E−03 | 2.77 | 51 |
| 403545 | | | Target Exon | 2.012E−03 | 2.77 | 43 |
| 416666 | H73028 | Hs. 268992 | ESTs | 1.549E−03 | 2.74 | 32.25 |
| 447981 | R53772 | Hs. 8929 | hypothetical protein FLJ11362 | 4.160E−02 | 2.74 | 37 |
| 453560 | AA348626 | Hs. 5890 | hypothetical protein FLJ23306 | 4.700E−02 | 2.74 | 41.75 |
| 441269 | AW015206 | Hs. 178784 | ESTs | 4.422E−02 | 2.72 | 64 |
| 441427 | BE550625 | Hs. 126956 | ESTs | 4.030E−04 | 2.72 | 34 |
| 411283 | AW852754 | | gb: PM1-CT0247-180100-009-c05 CT0247 Homo | 1.337E−02 | 267 | 33.5 |
| 423433 | AK000497 | Hs. 128646 | hypothetical protein FLJ20639 | 7.487E−02 | 2.66 | 34 |
| 421993 | R22497 | Hs. 110571 | growth arrest and DNA-damage-inducible, | 4.117E−05 | 2.65 | 371 |
| 442702 | AW235697 | Hs. 130980 | ESTs | 2.107E−02 | 2.65 | 38.25 |
| 414890 | BE281095 | Hs. 77573 | uridine phosphorylase | 1.084E−03 | 2.61 | 52.25 |
| 401346 | BE041451 | | hypothetical protein | 9.633E−03 | 2.60 | 46.5 |
| 408112 | AW451982 | Hs. 248613 | ESTs | 3.674E−02 | 2.60 | 40.75 |
| 432745 | AI821926 | | gb: nt78f05.x5 NCI_CGAP_Pr3 Homo sapiens | 4.904E−04 | 2.56 | 32.75 |
| 404717 | | | NM_007313*: Homo sapiens v-abl Abelson mu | 8.134E−02 | 2.56 | 30.75 |
| 437180 | BE180234 | Hs. 281462 | Homo sapiens cDNA FLJ14793 fis. clone NT | 5.867E−03 | 2.53 | 63 |
| 417213 | BE257508 | Hs. 24719 | modulator of apoptosis 1 | 2.923E−05 | 2.52 | 38.75 |
| 422743 | BE304678 | Hs. 119598 | ribosomal protein L3 | 1.837E−02 | 2.50 | 42.75 |
| 457148 | AF091035 | Hs. 184627 | KIAA0118 protein | 7.111E−03 | 2.50 | 60.25 |
| 417079 | U65590 | Hs. 81134 | interleukin 1 receptor antagonist | 1.839E−02 | 2.49 | 216.5 |
| 427899 | AA829286 | Hs. 332053 | serum amyloid A1 | 9.814E−05 | 2.48 | 799 |
| 427413 | BE547647 | Hs. 177781 | hypothetical protein MGC5618 | 1.159E−02 | 2.48 | 92.25 |
| 422924 | AI480125 | Hs. 185777 | ESTs | 9.609E−03 | 2.48 | 30.25 |
| 412429 | AV650262 | Hs. 75765 | GRO2 oncogene | 1.649E−06 | 2.47 | 246.25 |
| 407654 | AW064121 | Hs. 279175 | ESTs | 1.297E−03 | 2.46 | 40.5 |
| 447650 | AW581199 | Hs. 161137 | ESTs, Moderately similar to I54374 gene | 1.292E−02 | 2.45 | 40.5 |
| 434180 | AA921757 | Hs. 116180 | ESTs | 9.459E−04 | 2.45 | 50.25 |
| 414141 | BE255083 | Hs. 145729 | hypothetical protein DKFZp564A1164 | 6.099E−03 | 2.42 | 30.5 |
| 410204 | AJ243425 | Hs. 326035 | early growth response 1 | 1.419E−03 | 2.41 | 232 |
| 446912 | AI347650 | Hs. 128521 | ESTs, Moderately similar to ALU4_HUMAN A | 2.408E−02 | 2.40 | 37.25 |
| 448429 | D17408 | Hs. 21223 | calponin 1, basic, smooth muscle | 5.621E−02 | 2.40 | 31.5 |
| 423499 | AW608884 | Hs. 28068 | ESTs | 3.008E−04 | 2.40 | 31.75 |
| 424236 | AW058114 | Hs. 7837 | phosphoprotein regulated by mitogenic pa | 2.290E−03 | 2.37 | 47.5 |
| 426521 | AF161445 | Hs. 170219 | hypothetical protein | 2.841E−02 | 2.37 | 39.25 |
| 446378 | AI905699 | Hs. 239760 | citrate synthase | 8.200E−04 | 2.36 | 50.5 |
| 400818 | | | Target Exon | 3.204E−03 | 2.36 | 46.75 |
| 454339 | AW381980 | | gb: QV4-HT0316-091199-028-d05 HT0316 Homo | 1.969E−02 | 2.35 | 39 |
| 401942 | | | C17001396*: gi\|3212355\|pdb\|1A4P\|A Chain A | 1.759E−03 | 2.33 | 36 |
| 456147 | H41324 | Hs. 31581 | ESTs, Moderately similar to ST1B_HUMAN S | 6.882E−02 | 2.32 | 35.5 |
| 419305 | AI609195 | Hs. 304930 | ESTs | 5.144E−04 | 2.31 | 32.75 |
| 428403 | AI393048 | Hs. 326159 | leucine rich repeat (in FLII) interactin | 7.081E−02 | 2.30 | 52 |
| 456013 | T92048 | | gb: yd54g12.s1 Soares fetal liver spleen | 7.837E−04 | 2.29 | 30.25 |
| 435992 | AI033259 | Hs. 118317 | Homo sapiens cDNA FLJ12088 fis, clone HE | 2.663E−02 | 2.28 | 50.75 |
| 424272 | AA360929 | Hs. 144439 | putative RNA binding protein | 3.780E−03 | 2.28 | 34 |
| 411231 | AW833501 | | gb: QV4-TT0008-091199-025-e09 TT0008 Homo | 2.189E−03 | 2.27 | 30.5 |
| 434651 | BE616902 | Hs. 285313 | core promoter element binding protein | 9.321E−02 | 2.27 | 45.75 |
| 401376 | | | Target Exon | 1.773E−02 | 2.27 | 37.75 |
| 407338 | AA773213 | Hs. 200558 | gb: ab66f10.s1 Stratagene lung carcinoma | 1.038E−02 | 2.26 | 39.5 |
| 402012 | AF106069 | | ubiquitin specific protease 15 | 9.915E−04 | 2.25 | 36 |
| 415759 | AA169182 | Hs. 182740 | gb: zp20e02.s1 Stratagene fetal retina 93 | 9.630E−03 | 2.25 | 34.25 |
| 431387 | AI878854 | Hs. 252229 | v-maf musculoaponeurotic fibrosarcoma (a | 8.945E−03 | 2.23 | 35.5 |
| 452236 | AI130858 | Hs. 143218 | ESTs | 5.457E−02 | 2.22 | 31.25 |
| 405443 | | | Target Exon | 1.202E−02 | 2.21 | 132.75 |
| 402878 | | | ENSP00000217420*: BA122O1.1 (A novel prot | 1.132E−03 | 2.20 | 35.5 |
| 422831 | R02504 | Hs. 332943 | ESTs | 7.682E−03 | 2.20 | 39.75 |
| 410895 | AW809679 | | gb: MR4-ST0124-261099-015-f05 ST0124 Homo | 2.489E−02 | 2.20 | 34.75 |
| 405022 | | | Target Exon | 5.299E−02 | 2.19 | 33.75 |
| 403750 | | | C5001092: gi\|6671939\|gb\|AAF23199.1\|AC0167 | 4.249E−03 | 2.19 | 30.75 |
| 404187 | | | NM_019602: Homo sapiens butyrophilin-like | 9.022E−04 | 2.19 | 53.25 |
| 420701 | N42919 | Hs. 88630 | ESTs, Weakly similar to T14748 hypotheti | 9.286E−03 | 2.19 | 31.75 |
| 434495 | AW352170 | Hs. 129086 | Homo sapiens cDNA FLJ12007 fis, clone HE | 9.997E−03 | 2.16 | 38.5 |
| 427700 | AA262294 | Hs. 180383 | dual specificity phosphatase 6 | 3.340E−02 | 2.15 | 84 |
| 416348 | H65887 | Hs. 272163 | ESTs | 3.497E−04 | 2.15 | 30.5 |
| 426653 | AA530892 | Hs. 171695 | dual specificity phosphatase 1 | 3.337E−04 | 2.14 | 399.5 |
| 440249 | AI246590 | Hs. 249175 | ESTs | 9.290E−05 | 2.14 | 53.75 |
| 458568 | AI769067 | Hs. 127824 | ESTs, Weakly similar to T28770 hypotheti | 1.340E−02 | 2.11 | 395.75 |
| 459711 | BE386801 | Hs. 21858 | trinucleotide repeat containing 3 | 4.562E−02 | 2.10 | 37.5 |
| 409540 | AW409569 | | gb: fh01e09.x1 NIH_MGC_17 Homo sapiens cD | 2.287E−03 | 2.09 | 41.75 |
| 437389 | AL359587 | Hs. 271586 | hypothetical protein DKFZp762M115 | 3.208E−03 | 2.09 | 87.75 |
| 455968 | BE168828 | | gb: QV1-HT0517-020400-145-f04 HT0517 Homo | 1.237E−03 | 2.08 | 50.5 |
| 419909 | AL136653 | Hs. 93675 | decidual protein induced by progesterone | 2.488E−03 | 2.07 | 80.25 |
| 407869 | AI827976 | Hs. 24391 | hypothetical protein FLJ13612 | 1.358E−03 | 2.07 | 199 |
| 456465 | M94065 | Hs. 94925 | dihydroorotate dehydrogenase | 5.155E−04 | 2.07 | 121.75 |
| 412541 | BE009398 | Hs. 74002 | nuclear receptor coactivator 1 | 9.647E−03 | 2.04 | 41.25 |
| 423935 | BE382429 | Hs. 268561 | hypothetical protein FLJ10726 | 1.246E−02 | 2.03 | 38.75 |
| 408022 | AW137208 | Hs. 176000 | ESTs | 2.571E−02 | 2.03 | 42.25 |
| 406140 | | | Target Exon | 1.081E−03 | 2.03 | 48 |
| 417076 | AW973454 | Hs. 238442 | ESTs, Moderately similar to ALU7_HUMAN A | 9.630E−03 | 2.02 | 31.5 |
| 442321 | AF207664 | Hs. 8230 | a disintegrin-like and metalloprotease ( | 1.926E−04 | 2.02 | 187 |
| 431111 | AB033072 | Hs. 250015 | KIAA1246 protein | 1.272E−04 | 2.01 | 37.25 |
| 445132 | Z44811 | Hs. 14928 | hypothetical protein FLJ12903 | 5.935E−04 | 2.01 | 49 |
| 438808 | M73980 | Hs. 129053 | Homo sapiens NOTCH 1 (N1) mRNA, complete | 2.703E−03 | 2.01 | 34.25 |
| 406210 | | | Target Exon | 2.179E−02 | 2.01 | 34.5 |
| 422424 | AI186431 | Hs. 296638 | prostate differentiation factor | 2.333E-02 | 2.00 | 153.5 |

Pkey: Unique Eos probeset identifier number
ExAccn: Exemplar Accession number, Genbank accession number
UnigeneID: Unigene number
Unigene Title: Unigene gene title
R1: Wilcoxon rank-sum test p-value
R2: Trimean of Hep C− Liver over Trimean of Hep C+ liver Ratio
R3: Difference of Trimean of Hep C− Liver vs. Trimean of Hep C+ Liver

TABLE 4B

| Pkey | CAT Number | Accession |
|---|---|---|
| 409540 | 1138613_1 | AW409569 BE297044 BE295828 |
| 409840 | 1156071_1 | AW502122 AW502125 AW501663 AW501720 |
| 410895 | 1226051_1 | AW809679 AW809678 AW810113 AW810182 AW809900 AW809851 AW810110 AW810228 AW810342 AW810181 |
| | | AW809632 AW809745 |
| | | AW810372 AW809681 AW809792 AW809806 AW810452 AW809675 AW809964 AW810033 AW810111 |
| | | AW809846 AW809847 AW809717 |
| 411231 | 1236356_1 | AW833501 AW833506 AW833722 AW833332 AW833509 AW833511 AW833767 AW833339 |
| 411283 | 1237666_1 | AW852754 AW852897 AW852757 AW852617 BE172755 AW835444 |
| 411349 | 1239900_1 | AW838313 AW838353 AW838382 AW838207 |
| 412176 | 1281370_1 | AW898334 H66426 AW899792 |
| 417732 | 1695922_1 | R36065 R12062 R12616 Z43412 |
| 419555 | 185884_1 | AA244416 AA244401 |
| 423053 | 224319_1 | BE312679 BE314223 AA320990 |
| 429258 | 301917_1 | AA448765 C04967 C03045 AA658293 |
| 432745 | 353673_1 | AI821926 AA658826 AA564492 AA635129 AI791191 |
| 433010 | 357372_1 | AW970018 AA573669 AA573622 R08736 |
| 446052 | 65988_1 | AA358760 AA158850 AW062737 AW062738 AV656291 |
| 447135 | 70963_1 | T58148 AW516579 AW059603 |
| 447721 | 73383_1 | BE619620 AA448721 AI971709 AW175624 W24193 W24199 BE463718 AW193322 AI341487 AW072844 |
| | | BE048584 AW593800 AI623222 |
| | | AI983635 AW275114 AI952164 AI800442 AA385255 AW959076 AA977038 AI801910 AW513859 AW273202 AW337946 |
| | | AA250733 AW273147 |
| | | AI453134 AW235230 AI818468 AW166266 AI086791 AW300481 AI631778 AI561259 AW470887 AI207341 |
| | | AW103087 AW193240 AI052433 |
| | | AI128968 AW264492 AA327876 AI682412 AW771868 AI669677 AA448630 AA456607 AI128695 AI825128 |
| | | AW195959 N79049 AI470892 |
| | | AA902669 AI672486 AA769789 AI214684 |
| 449986 | 821463_1 | AW864502 AW864369 AI678780 |
| 450799 | 847242_1 | AW407504 W31274 AI738877 |
| 454339 | 1122972_1 | AW381980 BE152244 BE152235 BE152238 BE152232 |
| 454598 | 1226062_1 | AW809716 AW810152 AW809646 AW809747 AW809738 AW809826 AW809996 AW809798 AW809802 AW809840 |
| | | AW809695 |
| 454636 | 1227589_1 | AW811502 AW811521 AW811548 AW811471 AW811511 AW811508 |
| 454985 | 1248105_1 | AW849292 AW849431 AW849422 AW849428 AW849420 AW849424 AW849427 |
| 455968 | 1391117_1 | BE168828 BE168830 BE168823 BE168928 BE168820 BE168826 |
| 456013 | 1411982_1 | T92048 BE242744 |
| 458867 | 80304_1 | AW995393 AJ403118 |

Pkey: Unique Eos probeset identifier number
CAT number: Gene cluster number
Accession: Genbank accession numbers

TABLE 4C

| Pkey | Ref | Strand | Nt_position |
|---|---|---|---|
| 400818 | 8569994 | Plus | 172644-172765, 173085-173200 |
| 401149 | 7229925 | Plus | 73117-74019 |
| 401346 | 9926605 | Minus | 12031-13032 |
| 401376 | 7417809 | Plus | 40584-40963 |
| 401677 | 9965537 | Minus | 62856-63086, 63603-63884 |
| 401913 | 9369520 | Minus | 33753-33904 |
| 401942 | 4982556 | Minus | 130749-131044 |
| 402012 | 7407997 | Minus | 111771-111909, 112107-112226, |
| | | | 112519-112694 |
| 402070 | 8117335 | Minus | 68256-68484 |
| 402124 | 4033680 | Plus | 164206-164459 |
| 402273 | 2979528 | Plus | 28990-29203, 32299-32402, |
| | | | 32474-32668 |
| 402517 | 9798106 | Plus | 17569-17721 |
| 402651 | 7960391 | Plus | 174215-174380 |
| 402663 | 8077020 | Minus | 14155-14364 |
| 402777 | 9588235 | Plus | 126786-126948 |
| 402878 | 9908870 | Minus | 56133-56522 |
| 402889 | 9931133 | Plus | 89392-89498, 90358-90571 |
| 403180 | 7523976 | Minus | 63603-63759 |
| 403324 | 8440025 | Minus | 107104-107309 |
| 403508 | 7630896 | Plus | 5570-5719 |
| 403545 | 8078400 | Plus | 25293-25640 |
| 403579 | 8101179 | Minus | 36167-36365 |
| 403582 | 8101186 | Plus | 18308-18458 |
| 403750 | 7229814 | Minus | 133638-134110 |
| 403794 | 8096910 | Plus | 163292-163884 |
| 403796 | 8099896 | Minus | 75073-77664 |
| 404187 | 4481839 | Plus | 7644-7991 |
| 404501 | 7229859 | Minus | 37270-37526 |
| 404717 | 9838068 | Minus | 165900-166052, 169415-169599, |
| | | | 171430-171607, 172254-172338 |
| 404958 | 7407941 | Minus | 2731-4531 |
| 405022 | 7330304 | Plus | 217163-217439 |
| 405278 | 6139075 | Minus | 3863-3965, 4823-4891, 5439-5529, |
| | | | 6043-6170, 6344-6562, 7835-7990 |
| 405443 | 7408143 | Plus | 90716-90887, 101420-101577 |
| 405600 | 5923640 | Plus | 26662-27225 |
| 406140 | 9168231 | Minus | 49887-50219 |
| 406210 | 7341959 | Plus | 58546-58687 |

Pkey: Unique number corresponding to an Eos probeset
Ref: Sequence source. The 7 digit numbers in this column are Genbank Identifier (GI) numbers. “Dunham, et al.” refers to the publication entitled “The DNA sequence of human chromosome 22” Dunham, et al. (1999) Nature 402: 489-495.
Strand: Indicates DNA strand from which exons were predicted.
Nt_position: Indicates nucleotide positions of predicted exons.

Table 5A lists about 164 genes up-regulated in Hepatitis C positive liver tissues at a time before pegylated-interferon-alpha plus ribavirin treatment that was found to be non-responsive (non-responders) to the treatment compared to Hepatitis C positive liver tissues at a time before pegylated-interferon-alpha plus ribavirin treatment that was found to be responsive to the treatment (responders). These were selected from 59680 probesets on the Affymetrix/Eos Hu03 GeneChip array such that the Wilcoxon rank-sum test p-value between the 2 groups was less than 0.10, the ratio of the “weighted average” of non-responders to the “weighted average” of responders was equal to or above 2.0, and that the differences between the same 2 groups was equal to or above 30.0. The “weighted average” of the non-responders was set to the trimean of the non-responders. The “weighted average” of the responders was set to the either 10 or the trimean of the different responders, whichever value was greater to eliminate ratios with a denominator of zero or less. Tables 5B and 5C relate to Table 5A as 3B and 3C relate to 3A.

TABLE 5A

| Pkey | ExAccn | UnigeneID | Unigene Title | R1 | R2 | R3 |
|---|---|---|---|---|---|---|
| 454075 | R43826 | Hs. 16313 | Kruppel-like zinc finger protein GLIS2 | 6.136E−02 | 7.15 | 64.75 |
| 430273 | AI311127 | Hs. 125522 | ESTs | 7.404E−02 | 6.35 | 60 |
| 444342 | NM_014398 | Hs. 10887 | similar to lysosome−associated membrane | 5.766E−03 | 6.25 | 77.5 |
| 424381 | AA285249 | Hs. 146329 | protein kinase Chk2 | 3.476E−02 | 6.17 | 52.75 |
| 444104 | AW296150 | Hs. 177151 | ESTs | 7.353E−02 | 5.97 | 57 |
| 447261 | NM_006691 | Hs. 17917 | extracellular link domain-containing 1 | 5.091E−02 | 5.33 | 53 |
| 426990 | AL044315 | Hs. 173094 | Homo sapiens mRNA for KIAA1750 protein, | 7.404E−02 | 5.31 | 52.75 |
| 404493 | NA | | Target Exon | 1.485E−02 | 5.13 | 42 |
| 412747 | AW994222 | | gb: RC3-BN0036-250200-012-e02 BN0036 Homo | 5.091E−02 | 5.07 | 51.75 |
| 404075 | NA | | Target Exon | 5.132E−02 | 4.97 | 41 |
| 454525 | BE280421 | Hs. 94499 | ESTs | 5.681E−03 | 4.95 | 50.5 |
| 445098 | AL050272 | Hs. 12305 | DKFZP566B183 protein | 1.812E−02 | 4.92 | 39.25 |
| 402012 | AF106069 | | ubiquitin specific protease 15 | 7.404E−02 | 4.91 | 44 |
| 452826 | BE245286 | Hs. 301636 | peroxisomal biogenesis factor 6 | 2.299E−02 | 4.91 | 62.5 |
| 407828 | AW959500 | Hs. 49597 | retinoic acid induced 2 | 4.200E−02 | 4.75 | 49.5 |
| 417123 | BE326521 | Hs. 159450 | ESTs | 3.381E−02 | 4.70 | 43.25 |
| 418483 | W26076 | Hs. 221847 | ESTs | 5.132E−02 | 4.70 | 41.25 |
| 414308 | AI740705 | Hs. 126485 | hypothetical protein FLJ12604; KIAA1692 | 5.051E−02 | 4.55 | 42.25 |
| 426106 | AI678765 | Hs. 21812 | ESTs | 3.476E−02 | 4.49 | 53.25 |
| 437233 | D81448 | Hs. 339352 | Homo sapiens brother of CDO (BOC) mRNA, | 6.136E−02 | 4.47 | 59.25 |
| 403807 | NA | | NM_031889: Homo sapiens enamelin (ENAM), | 5.681E−03 | 4.34 | 36.75 |
| 432250 | AA452088 | Hs. 274170 | Opa-interacting protein 2 | 1.833E−02 | 4.32 | 39.75 |
| 452455 | N25153 | Hs. 61661 | ESTs, Weakly similar to AF174605 1 F-box | 2.251E−02 | 4.25 | 40 |
| 459171 | AW967801 | Hs. 64783 | ESTs, Weakly similar to T42705 hypotheti | 5.132E−02 | 4.17 | 35.5 |
| 435791 | AA243086 | Hs. 25204 | chondroitin 4-O-sulfotransferase 2 | 1.485E−02 | 4.17 | 41.25 |
| 426798 | AA385062 | Hs. 130260 | ESTs | 5.132E−02 | 4.13 | 32.5 |
| 424017 | AA333789 | | gb: EST37925 Embryo, 9 week Homo sapiens | 6.136E−02 | 4.10 | 35.25 |
| 446627 | AI973016 | Hs. 15725 | hypothetical protein SBBI48 | 3.476E−02 | 4.06 | 72 |
| 442269 | AI797066 | Hs. 201995 | ESTs | 1.450E−02 | 4.05 | 31 |
| 447698 | AI420156 | Hs. 326733 | ESTs | 1.168E−02 | 4.05 | 47.25 |
| 432336 | NM_002759 | Hs. 274382 | protein kinase, interferon-inducible dou | 7.404E−02 | 4.02 | 35 |
| 408711 | AW376061 | Hs. 63335 | ESTs, Moderately similar to A46010 X-lin | 7.404E−02 | 4.02 | 31 |
| 427867 | NM_005073 | Hs. 2217 | solute carrier family 15 (oligopeptide t | 8.877E−03 | 3.97 | 49.5 |
| 437990 | AI686579 | Hs. 121784 | ESTs | 5.132E−02 | 3.97 | 34 |
| 444640 | AL133933 | Hs. 64310 | interleukin 11 receptor, alpha | 7.353E−02 | 3.97 | 30.75 |
| 437063 | AA351109 | Hs. 5437 | Tax1 (human T-cell leukemia virus type I | 7.404E−02 | 3.95 | 33.25 |
| 428108 | AA421452 | | ESTs, Weakly similar to KIAA0926 protein | 2.807E−02 | 3.95 | 31.5 |
| 412055 | AA099907 | Hs. 271806 | ESTs, Weakly similar to ALU1_HUMAN ALU S | 5.091E−02 | 3.93 | 33 |
| 417936 | X01059 | Hs. 82963 | gonadotropin-releasing hormone 1 (leutin | 4.200E−02 | 3.92 | 37.5 |
| 410099 | AA081630 | | KIAA0036 gene product | 1.485E−02 | 3.91 | 80.75 |
| 410541 | AA065003 | Hs. 64179 | syntenin-2 protein | 5.766E−03 | 3.85 | 123.25 |
| 424259 | AK001776 | Hs. 143954 | hypothetical protein FLJ10914 | 2.299E−02 | 3.82 | 39.75 |
| 458381 | AI041873 | Hs. 132148 | ESTs | 5.132E−02 | 3.82 | 36.25 |
| 441897 | AI264686 | Hs. 23921 | hypothetical protein DKFZp547A023 | 5.091E−02 | 3.77 | 42.75 |
| 405411 | | | ENSP00000252213: SODIUM BICARBONATE COTRA | 5.132E−02 | 3.75 | 64.25 |
| 427509 | M62505 | Hs. 2161 | complement component 5 receptor 1 (C5a I | 3.476E−02 | 3.75 | 30.25 |
| 428151 | AA422028 | Hs. 171136 | ESTs | 3.476E−02 | 3.72 | 32 |
| 422553 | AI697720 | Hs. 171455 | ESTs, Weakly similar to T31613 hypotheti | 5.091E−02 | 3.69 | 78 |
| 401842 | NA | | Target Exon | 7.404E−02 | 3.67 | 38 |
| 433365 | AF026944 | Hs. 293797 | ESTs | 5.091E−02 | 3.65 | 68 |
| 402200 | AL080200 | | DKFZP434F122 protein | 2.275E−02 | 3.65 | 41 |
| 437191 | NM_006846 | Hs. 331555 | serine protease inhibitor, Kazal type, 5 | 6.136E−02 | 3.63 | 31.75 |
| 444196 | AW139633 | Hs. 279582 | GTP-binding protein Sara | 5.132E−02 | 3.60 | 35 |
| 403436 | | | NM_006548*: Homo sapiens IGF-II mRNA-bind | 2.299E−02 | 3.59 | 39.5 |
| 435128 | AF176832 | Hs. 47005 | low density lipoprotein-related protein | 5.681E−03 | 3.57 | 39.25 |
| 422240 | R60594 | Hs. 29002 | KIAA1706 protein | 5.132E−02 | 3.57 | 32.75 |
| 431726 | NM_015361 | Hs. 268053 | KIAA0029 protein | 3.444E−02 | 3.52 | 30.25 |
| 407366 | AF026942 | Hs. 271530 | gb: Homo sapiens cig33 mRNA, partial sequ | 1.485E−02 | 3.51 | 91 |
| 404109 | | | C6000844*: gi\|7497891\|pir\|\|T20194 hypothe | 1.468E−02 | 3.50 | 33 |
| 426788 | U66615 | Hs. 172280 | SWI/SNF related, matrix associated, acti | 7.404E−02 | 3.46 | 32 |
| 415822 | D59243 | | gb: HUM526E07B Clontech human placenta po | 2.299E−02 | 3.45 | 52.25 |
| 449922 | AA004731 | | gb: zh93g05.s1 Soares_fetal_liver_spleen_{—} | 5.132E−02 | 3.43 | 32.75 |
| 458159 | AA883831 | Hs. 252924 | ESTs | 9.243E−03 | 3.42 | 44.5 |
| 446650 | AB016625 | Hs. 15813 | solute carrier family 22 (organic cation | 5.132E−02 | 3.42 | 38.25 |
| 450011 | AW071789 | Hs. 99233 | ESTs | 5.681E−03 | 3.42 | 38 |
| 432706 | NM_013230 | Hs. 286124 | CD24 antigen (small cell lung carcinoma | 8.760E−02 | 3.42 | 34.5 |
| 447898 | AW969638 | Hs. 112318 | 6.2 kd protein | 3.476E−02 | 3.40 | 46.75 |
| 439478 | AF049460 | Hs. 6574 | deformed epidermal autoregulatory factor | 3.476E−02 | 3.36 | 50.25 |
| 414709 | AA704703 | Hs. 77031 | Sp2 transcription factor | 2.299E−02 | 3.35 | 63.5 |
| 450404 | AA742544 | Hs. 159231 | hypothetical protein FLJ21551 | 5.132E−02 | 3.35 | 34 |
| 429505 | AW820035 | Hs. 278679 | a disintegrin and metalloproteinase doma | 3.476E−02 | 3.25 | 32.25 |
| 440258 | AI741633 | Hs. 125350 | ESTs | 9.243E−03 | 3.24 | 32.5 |
| 449713 | AW027025 | Hs. 345528 | ESTs | 3.476E−02 | 3.24 | 38 |
| 432791 | NM_014554 | Hs. 66450 | sentrin/SUMO-specific protease | 7.404E−02 | 3.22 | 38.5 |
| 408570 | AL046406 | Hs. 103483 | KIAA1798 protein | 7.302E−02 | 3.22 | 38 |
| 403512 | NA | | C3000579*: gi\|12643308\|sp\|Q9Y4K1\|AIM1_HUM | 1.485E−02 | 3.13 | 46.75 |
| 406303 | | | C16000922: gi\|7499103\|pir\|\|T20903 hypothe | 3.444E−02 | 3.07 | 30.5 |
| 449137 | AW134478 | Hs. 196033 | ESTs | 7.268E−03 | 3.05 | 52 |
| 401807 | | | C7001350: gi\|6578126\|gb\|AAF17706.1\|AF0496 | 5.132E−02 | 3.05 | 31.5 |
| 423748 | AI149048 | Hs. 30211 | hypothetical protein FLJ22313 | 5.132E−02 | 3.02 | 38 |
| 448497 | BE613269 | Hs. 21893 | hypothetical protein DKFZp761N0624 | 1.168E−02 | 3.02 | 34.25 |
| 447164 | AF026941 | Hs. 17518 | Homo sapiens cig5 mRNA, partial sequence | 5.132E−02 | 2.98 | 128 |
| 450446 | AI696334 | Hs. 14450 | ESTs | 1.468E−02 | 2.97 | 43.75 |
| 435938 | AI248774 | Hs. 126707 | hypothetical protein FLJ11457 | 4.970E−02 | 2.97 | 35.5 |
| 422744 | AW268803 | Hs. 119640 | hBKLF for basic kruppel like factor | 4.128E−02 | 2.97 | 34.5 |
| 453397 | AA035378 | Hs. 31222 | ESTs, Weakly similar to DRPL_HUMAN ATROP | 5.132E−02 | 2.96 | 34.25 |
| 454204 | AW816498 | | gb: QV0-ST0236-171299-075-b02 ST0236 Homo | 3.444E−02 | 2.95 | 30.25 |
| 443041 | AA558677 | Hs. 8928 | hypothetical protein FLJ20291 | 2.299E−02 | 2.93 | 40.5 |
| 422366 | T83882 | Hs. 97927 | ESTs | 4.164E−02 | 2.92 | 31.75 |
| 429982 | AW449534 | Hs. 99607 | hypothetical protein FLJ13841 | 9.366E−03 | 2.77 | 51.25 |
| 447278 | AI934935 | Hs. 158669 | ESTs | 8.760E−02 | 2.77 | 33.75 |
| 431250 | BE264649 | Hs. 251377 | taxol resistance associated gene 3 | 7.404E−02 | 2.76 | 85.5 |
| 419270 | NM_005232 | Hs. 89839 | EphA1 | 7.404E−02 | 2.76 | 45.75 |
| 425109 | R37456 | Hs. 184793 | Homo sapiens cDNA: FLJ21880 fis, clone H | 3.349E−02 | 2.75 | 35 |
| 417214 | AW192804 | Hs. 24719 | modulator of apoptosis 1 | 5.766E−03 | 2.74 | 52.25 |
| 430259 | BE550182 | Hs. 127826 | RalGEF-like protein 3, mouse homolog | 5.766E−03 | 2.74 | 37.75 |
| 427765 | AA412247 | Hs. 111970 | ESTs | 5.132E−02 | 2.72 | 33.5 |
| 409947 | AA078629 | Hs. 27301 | ESTs | 3.444E−02 | 2.72 | 37 |
| 443493 | AI074053 | Hs. 127146 | ESTs | 3.349E−02 | 2.71 | 35 |
| 449394 | AA004368 | Hs. 18160 | Homo sapiens cDNA FLJ11550 fis. clone HE | 3.476E−02 | 2.70 | 45.75 |
| 405348 | NA | | C7001664: gi\|12698061\|dbj\|BAB21849.1\| (AB | 3.476E−02 | 2.70 | 42.75 |
| 448030 | N30714 | Hs. 325960 | membrane-spanning 4-domains, subfamily A | 1.468E−02 | 2.68 | 39 |
| 418203 | X54942 | Hs. 83758 | CDC28 protein kinase 2 | 5.132E−02 | 2.66 | 42.75 |
| 422833 | BE141312 | | gb: MR0-HT0078-051099-002-h06 HT0078 Homo | 1.468E−02 | 2.65 | 38.75 |
| 448124 | AI470454 | Hs. 199150 | ESTs | 3.444E−02 | 2.60 | 37 |
| 401536 | | | NM_002530*: Homo sapiens neurotrophic tyr | 8.760E−02 | 2.53 | 41.75 |
| 456370 | AA234938 | Hs. 87384 | ESTs | 3.476E−02 | 2.50 | 52.25 |
| 404607 | NA | | Target Exon | 5.132E−02 | 2.50 | 48.5 |
| 436729 | BE621807 | | transmembrane 4 superfamily member 1 | 7.353E−02 | 2.49 | 35 |
| 404801 | | | NM_004286*: Homo sapiens GTP binding prot | 2.299E−02 | 2.46 | 50.25 |
| 414173 | N27495 | Hs. 5565 | hypothetical protein FLJ22626 | 2.275E−02 | 2.45 | 39.75 |
| 413719 | BE439580 | Hs. 75498 | small inducible cytokine subfamily A (Cy | 3.476E−02 | 2.45 | 64 |
| 413550 | W03011 | Hs. 306881 | MSTP043 protein | 8.705E−02 | 2.44 | 32.75 |
| 405416 | | | C19001058*: gi\|5080758\|gb\|AAD39268.1\|AC00 | 3.476E−02 | 2.42 | 31.25 |
| 458388 | W28887 | Hs. 133142 | Homo sapiens cDNA: FLJ22153 fis, clone H | 7.404E−02 | 2.42 | 30.5 |
| 432886 | BE159028 | Hs. 279704 | chromatin accessibility complex 1 | 1.485E−02 | 2.41 | 34.25 |
| 419497 | NM_006410 | Hs. 90753 | Tat-interacting protein (30 kD) | 8.760E−02 | 2.40 | 33.5 |
| 443743 | AI084210 | Hs. 303925 | ESTs, Weakly similar to A46010 X-linked | 8.760E−02 | 2.39 | 34.5 |
| 444811 | AW137791 | Hs. 148419 | ESTs | 1.485E−02 | 2.39 | 41 |
| 430291 | AV660345 | Hs. 238126 | CGI-49 protein | 7.404E−02 | 2.39 | 41 |
| 459119 | AW844498 | Hs. 306121 | CDC20 (cell division cycle 20, S. cerevi | 5.132E−02 | 2.38 | 38.75 |
| 432195 | AJ243669 | Hs. 8127 | KIAA0144 gene product | 3.476E−02 | 2.37 | 44.75 |
| 416046 | H16054 | Hs. 297644 | ESTs | 8.705E−02 | 2.36 | 46.5 |
| 421650 | AA781795 | Hs. 122587 | ESTs | 3.444E−02 | 2.35 | 59.5 |
| 455886 | BE153549 | Hs. 293818 | ribosomal protein L7 | 5.132E−02 | 2.34 | 40 |
| 409775 | AW499605 | | gb: UI-HF-BP0p-ain-c-05-O-Ul.r1 NIH_MGC_5 | 5.132E−02 | 2.32 | 49 |
| 402601 | | | Target Exon | 5.132E−02 | 2.31 | 55.5 |
| 439602 | W79114 | Hs. 58558 | ESTs | 7.353E−02 | 2.30 | 41 |
| 458946 | AA009716 | Hs. 42311 | ESTs | 6.136E−02 | 2.30 | 32.75 |
| 419445 | AA884471 | Hs. 90449 | Human clone 23908 mRNA sequence | 3.476E−02 | 2.30 | 31.5 |
| 439242 | BE167324 | Hs. 53996 | ESTs, Weakly similar to ZN42_HUMAN ZINC | 9.243E−03 | 2.29 | 50.5 |
| 444301 | AK000136 | Hs. 10760 | asporin (LRR class 1) | 5.132E−02 | 2.28 | 50.75 |
| 435706 | W31254 | Hs. 7045 | GL004 protein | 7.404E−02 | 2.27 | 46.5 |
| 405418 | NA | | Target Exon | 5.766E−03 | 2.27 | 53.25 |
| 452032 | BE244005 | Hs. 27610 | retinoic acid- and interferon-inducible | 7.268E−03 | 2.25 | 53.5 |
| 444830 | AI198854 | Hs. 145437 | ESTs | 6.136E−02 | 2.24 | 32 |
| 428075 | AW205525 | Hs. 212511 | ESTs | 5.132E−02 | 2.23 | 48.75 |
| 446169 | AA398857 | Hs. 14142 | nudix (nucleoside diphosphate linked moi | 9.366E−03 | 2.23 | 31.25 |
| 453405 | AI567972 | | ESTs, Highly similar to AF161437 1 HSPC3 | 7.302E−02 | 2.22 | 48 |
| 441942 | AF182645 | Hs. 8024 | IK cytokine, down-regulator of HLA II | 7.404E−02 | 2.19 | 38.5 |
| 417640 | D30857 | Hs. 82353 | protein C receptor, endothelial (EPCR) | 7.353E−02 | 2.19 | 37.5 |
| 425225 | NM_003450 | Hs. 155204 | zinc finger protein 174 | 2.251E−02 | 2.18 | 40.25 |
| 430227 | AI924441 | Hs. 236218 | TAT-INTERACTIVE PROTEIN, 72-KD | 7.404E−02 | 2.17 | 49.5 |
| 458704 | AI343613 | Hs. 156600 | ESTs | 4.200E−02 | 2.17 | 33.25 |
| 404973 | NA | | Target Exon | 5.091E−02 | 2.16 | 47.25 |
| 448362 | AA641767 | Hs. 21015 | hypothetical protein DKFZp564L0864 simil | 1.485E−02 | 2.14 | 37 |
| 428315 | AA688152 | Hs. 98505 | ESTs | 6.091E−02 | 2.13 | 44.75 |
| 415749 | BE262529 | Hs. 78771 | phosphoglycerate kinase 1 | 7.166E−03 | 2.12 | 39 |
| 419104 | AA709154 | Hs. 191514 | ESTs | 5.132E−02 | 2.11 | 33 |
| 437849 | AA769680 | Hs. 18768 | ESTs | 3.476E−02 | 2.11 | 36.25 |
| 428726 | AA432195 | Hs. 98694 | ESTs | 5.132E−02 | 2.09 | 43.25 |
| 440495 | AA887212 | Hs. 14161 | hypothetical protein DKFZp43411930 | 1.485E−02 | 2.08 | 41.75 |
| 446337 | AW272746 | Hs. 239818 | phosphoinositide-3-kinase, catalytic, be | 5.132E−02 | 2.07 | 39.5 |
| 404975 | AL042279 | | uncharacterized hypothalamus protein HT0 | 7.302E−02 | 2.06 | 38.25 |
| 415599 | Z44487 | Hs. 8763 | gb: HSC21F081 normalized infant brain cDN | 7.353E−02 | 2.05 | 34.75 |
| 416404 | AA180138 | Hs. 107924 | ESTs | 7.404E−02 | 2.05 | 41.5 |
| 439277 | R80061 | Hs. 164478 | hypothetical protein FLJ21939 similar to | 8.649E−02 | 2.05 | 33.75 |
| 430108 | AA465294 | Hs. 291750 | ESTs | 1.485E−02 | 2.05 | 31.5 |
| 422746 | NM_004484 | Hs. 119651 | glypican 3 | 7.404E−02 | 2.02 | 144.5 |
| 458410 | H20380 | Hs. 200250 | ESTs, Moderately similar to ALU7_HUMAN A | 8.760E−02 | 2.01 | 39.25 |
| 410204 | AJ243425 | Hs. 326035 | early growth response 1 | 1.485E−02 | 2.01 | 148.75 |
| 441616 | BE569122 | | RNA-binding protein (autoantigenic) | 5.091E−02 | 2.01 | 31.25 |
| 414395 | BE304888 | Hs. 279834 | EST | 7.353E−02 | 2.00 | 40.25 |
| 456999 | AA319798 | Hs. 298581 | eukaryotic translation elongation factor | 3.476E−02 | 2.00 | 31.25 |

Pkey: Unique Eos probeset identifier number
ExAccn: Exemplar Accession number, Genbank accession number
UnigeneID: Unigene number
Unigene Title: Unigene gene title
R1: Wilcoxon rank-sum test p-value
R2: Trimean of non-responders over Trimean of responders Ratio
R3: Difference of Trimean of non-responders vs. Trimean of responders

TABLE 5B

| Pkey | CAT Number | Accession |
|---|---|---|
| 409775 | 1154112_1 | AW499605 AW501372 AW503065 AW500923 AW501639 |
| 410099 | 117647_1 | AA081630 T08671 AI174254 D83874 AW959843 AA364503 |
| | | AA693467 AW993370 BE327037 AA167714 N79906 AW901977 AW901980 |
| | | W52882 T07735 AA484549 W60090 D52685 T23811 BE327043 AW901768 BE551237 |
| | | AA917004 AA716027 AI439658 AA283724 AI805992 |
| | | AI457096 AA084618 BE467736 AI092635 AI887863 AI697593 AA436618 AI167419 |
| | | AI418634 T31586 AA436630 AA706191 AI041169 |
| | | AI422304 T03534 AA211402 AI204899 AI366472 AW827081 AA788593 T32736 |
| | | AI767935 AA167791 AA747914 AA663870 AI865504 |
| 412747 | 1324696_1 | AW994222 AW994377 |
| 415822 | 155791_1 | D59243 D63202 AA169716 |
| 422833 | 221904_1 | BE141312 BE141417 BE141389 AA317747 |
| 424017 | 234338_1 | AA333789 AA334317 AW966107 |
| 428108 | 287154_1 | AA421452 BE302040 AI204456 AA833751 AA421483 AI825059 |
| 436729 | 42585_1 | BE621807 AI445461 AI346835 AI453743 AI564644 AI928364 AW984527 BE156214 |
| | | AI694111 AI591358 C17504 C17476 C17963 C18304 |
| | | AW071625 AI678712 C17732 D57559 H61762 AI720939 AI262930 H27252 AA479712 |
| | | AI927769 AA291465 AA155661 AI963432 AI567995 |
| | | AA421678 AI925607 AA292956 AA192448 AW192593 AI865838 AI696905 AI871950 |
| | | AI911921 BE619741 BE439796 AI161312 AI597801 |
| | | AI424384 AI093510 AI240988 AW820230 AI492554 BE044033 AW262737 AW008570 |
| | | AA043216 AW629505 AA136645 AA037722 AA706057 |
| | | AA088439 AW806193 AW806183 AA479834 BE501957 AA129574 R38114 AA649494 |
| | | AA524526 BE327120 AW572531 BE219784 BE349186 |
| | | AW015724 AA043217 AW772000 AI799814 AI671727 AW779725 AA502832 AI470033 |
| | | AA129575 W38161 AI972739 AA404570 AA627686 |
| | | AA723200 AA147228 AA903050 AI990245 AI075878 T32487 C06123 AA157944 AI800106 |
| | | W60075 AI859160 AA478328 AW673152 AA182640 |
| | | AI990827 AW275048 AW103470 AI298935 AW471421 R79190 AW085158 W45410 |
| | | AW300456 AA662517 T55840 AI823466 |
| | | AI692846 AA962397 AW191997 AA136658 AI251817 BE044134 AW339104 AW517762 AA724739 |
| | | R79933 AA411100 AA191349 AA037696 |
| | | AA190966 AA757735 AW772283 AA010631 H80983 AI769516 H64985 AI061065 AI950693 |
| | | AA085492 AI245632 H28594 AW088968 BE156360 |
| | | AI349390 AI621320 AI738844 AW194272 AA148284 AA953883 C06365 AA487893 |
| | | AI927217 AI918523 AI453453 AI798502 AI189366 |
| | | AI261359 AI032569 AW338678 AI972899 AI500576 AI872628 AI693030 Z28771 |
| | | AI985583 AI363829 AW339301 AA581093 AI650338 W60032 |
| | | AA603586 AI686240 AW242958 AA719173 AI745717 AW675302 AI582462 AI244845 |
| | | AI565439 F09579 AI918453 AA035576 AI472527 |
| | | AW351556 AA191414 AW674145 D57558 AI446740 D57845 AI589264 C05782 AA722206 |
| | | AI432033 R21752 BE157510 AI829640 AI468237 |
| | | AW384233 AA989662 AI865912 AW197954 AI344941 X75684 AI344943 AW583310 AA988297 |
| | | AI334860 AI348877 AI798415 D11921 |
| | | AI377596 AI983655 AI744233 C06111 AI248307 AA948565 AI224807 |
| 441616 | 52161_2 | BE569122 BE43964 BE621682 BE548066 N73920 AA164369 W80481 |
| | | AI301065 N36665 AW071970 AW263496 AI499701 AA001425 |
| | | AA781414 AI479737 AA922117 AW104450 AA026632 N30835 AW274584 AW173171 AW817587 |
| | | R94765 AA019198 AA054252 H03499 |
| | | AA954721 AI926512 AI685317 AI393574 AW166832 AA742324 AI749979 AA972701 AA434451 |
| | | AI305153 AA678370 F12140 H16890 AI356800 |
| | | AW613806 R73589 AI280399 AW627592 AI334386 AI200095 W47269 AI491703 AA837589 AI073728 |
| | | AI367984 AI422897 AI282124 AA053948 |
| | | AI218584 H84116 AI273274 AA731678 AW338258 AI347082 AA256494 AW022998 |
| | | AA743354 AI698561 R69295 AI280442 AA480586 |
| | | AA026691 AI948431 AI381988 AA417566 BE564055 AA365458 AA054003 |
| 449922 | 81925_1 | AA004731 AA004787 AI240768 |
| 453405 | 96607_1 | AI567972 AW196160 AA035448 AA934760 N70743 AA862404 AA485737 |
| | | AA885091 AI926381 AA885115 AA284561 AA284560 C00839 |
| | | W01732 |
| 454204 | 1050597_1 | AW816498 AW808791 AW808515 AW808379 AW808532 AW808605 AW808977 |
| | | AW808816 AW178676 AW178486 AW808514 AW178483 |
| | | AW178485 AW809007 AW808524 |

Pkey: Unique Eos probeset identifier number
CAT number: Gene cluster number
Accession: Genbank accession numbers

TABLE 5C

| Pkey | Ref | Strand | Nt_position |
|---|---|---|---|
| 401536 | 7960358 | Plus | 100193-100336, 103486-103671 |
| 401807 | 7331536 | Plus | 152325-152912 |
| 401842 | 7684597 | Plus | 111602-111746 |
| 402012 | 7407997 | Minus | 111771-111909, 112107-112226, |
| | | | 112519-112694 |
| 402200 | 7689783 | Plus | 28877-29398 |
| 402601 | 7705237 | Minus | 170567-170936, 171175-171610, |
| | | | 173128-173238, 173317-173425, |
| | | | 173505-173766 |
| 403436 | 9719642 | Minus | 96248-96361, 98626-98757 |
| 403512 | 7656757 | Minus | 114487-114610 |
| 403807 | 8439933 | Minus | 162963-165773 |
| 404075 | 7652011 | Plus | 53692-53938 |
| 404109 | 9211742 | Minus | 144675-144928, 151899-152064 |
| 404493 | 8140670 | Plus | 38930-39208 |
| 404607 | 7328770 | Plus | 28477-28591 |
| 404801 | 4582132 | Plus | 89110-89482, 94224-94347 |
| 404973 | 3213020 | Plus | 101602-102591 |
| 404975 | 3419864 | Minus | 86096-86605 |
| 405348 | 2914717 | Minus | 43310-43462 |
| 405411 | 3451356 | Minus | 17503-17778, 18021-18290 |
| 405416 | 6984492 | Minus | 28380-30235 |
| 405418 | 6997292 | Plus | 51839-51953 |
| 406303 | 8575868 | Plus | 173622-173786 |

Pkey: Unique number corresponding to an Eos probeset
Ref: Sequence source. The 7 digit numbers in this column are Genbank Identifier (GI) numbers. “Dunham, et al.” refers to the publication entitled “The DNA sequence of human chromosome 22” Dunham, et al. (1999) Nature 402: 489-495.
Strand: Indicates DNA strand from which exons were predicted.
Nt_position: Indicates nucleotide positions of predicted exons.

Table 6A lists about 284 genes up-regulated in Hepatitis C positive liver tissues at a time before pegylated-interferon-alpha plus ribavirin treatment that was found to be responsive (responders) to the treatment compared to Hepatitis C positive liver tissues at a time before pegylated-interferon-alpha plus ribavirin treatment that was found to be non-responsive to the treatment (non-responders). These were selected from 59680 probesets on the Affymetrix/Eos Hu03 GeneChip array such that the Wilcoxon rank-sum test p-value between the 2 groups was less than 0.10, the ratio of the “weighted average” of responders to the “weighted average” of non-responders was equal to or above 2.0, and that the differences between the same 2 groups was equal to or above 30.0. The “weighted average” of the responders was set to the trimean of different non-responders. The “weighted average” of the non-responders was set to the either 10 or the trimean of different non-responders, whichever value was greater to eliminate ratios with a denominator of zero or less. Tables 6B and 6C relate to Table 6A as 3B and 3C relate to 3A.

TABLE 6A

| Pkey | ExAccn | UnigeneID | Unigene Title | R1 | R2 | R3 |
|---|---|---|---|---|---|---|
| 433156 | R59206 | Hs. 17519 | Homo sapiens cDNA: FLJ22539 fis, clone H | 9.366E−03 | 9.97 | 91.25 |
| 425701 | AA361850 | Hs. 322149 | Human clone 137308 mRNA, partial cds | 2.299E−02 | 8.90 | 86.75 |
| 453822 | NM_014116 | Hs. 35416 | PRO0132 protein | 7.404E−02 | 8.15 | 71.75 |
| 421160 | AL080215 | Hs. 102301 | Homo sapiens mRNA; cDNA DKFZp586J0323 (f | 8.705E−02 | 7.32 | 74.5 |
| 424375 | AF070547 | Hs. 146312 | Homo sapiens clone 24820 mRNA sequence | 7.404E−02 | 7.27 | 75 |
| 435147 | AL133731 | Hs. 4774 | Homo sapiens mRNA; cDNA DKFZp761C1712 (f | 5.766E−03 | 6.52 | 104.5 |
| 416686 | H75435 | Hs. 169404 | ESTs | 3.476E−02 | 6.47 | 75.75 |
| 451078 | AI927694 | Hs. 340945 | ESTs | 5.132E−02 | 6.47 | 58.25 |
| 432474 | AA584042 | | gb: nn65e09.s1 NCI_CGAP_Lar1 Homo sapiens | 3.476E−02 | 6.40 | 54 |
| 415374 | F06904 | Hs. 8346 | ESTs | 2.299E−02 | 6.30 | 62.5 |
| 427958 | AA418000 | Hs. 98280 | potassium intermediate/small conductance | 5.681E−03 | 5.97 | 91 |
| 408806 | AW847814 | Hs. 289005 | Homo sapiens cDNA: FLJ21532 fis, clone C | 1.485E−02 | 5.82 | 54.75 |
| 401757 | | | Target Exon | 2.299E−02 | 5.77 | 59.75 |
| 400864 | | | Target Exon | 5.132E−02 | 5.63 | 55.5 |
| 432425 | AF070619 | Hs. 274539 | Homo sapiens clone 24481 mRNA sequence | 7.404E−02 | 5.60 | 65.5 |
| 401529 | | | Target Exon | 5.132E−02 | 5.57 | 47.75 |
| 451408 | AI795947 | Hs. 209596 | ESTs | 3.476E−02 | 5.55 | 60.5 |
| 444813 | AW054834 | Hs. 210356 | ESTs | 5.132E−02 | 5.40 | 57.5 |
| 418432 | M14156 | Hs. 85112 | insulin-like growth factor 1 (somatomedi | 7.404E−02 | 5.36 | 49 |
| 419831 | AW448930 | Hs. 5415 | ESTs | 7.353E−02 | 5.17 | 51.5 |
| 436593 | AW085002 | Hs. 156187 | ESTs | 2.698E−02 | 5.13 | 49 |
| 420315 | NM_006299 | Hs. 96448 | zinc finger protein 193 | 7.353E−02 | 5.05 | 54 |
| 438098 | AI076370 | Hs. 134037 | ESTs | 7.404E−02 | 5.05 | 43.5 |
| 411755 | BE327036 | Hs. 117494 | ESTs | 2.807E−02 | 5.02 | 44 |
| 442719 | H12048 | Hs. 91564 | ESTs | 8.705E−02 | 5.02 | 41 |
| 449625 | NM_014253 | | odz (odd Oz/ten-m, Drosophila) homolog 1 | 7.353E−02 | 4.92 | 63 |
| 407269 | AJ245210 | | gb: Homo sapiens mRNA for immunoglobulin | 4.164E−02 | 4.92 | 44.25 |
| 403609 | | | C3001199: gi\|7494834\|pir\|\|T15308 hypothet | 9.366E−03 | 4.82 | 51.25 |
| 421417 | AA291004 | Hs. 326088 | ESTs | 1.468E−02 | 4.82 | 40 |
| 400279 | | | NM_004581*: Homo sapiens Rab geranylgeran | 3.476E−02 | 4.81 | 44.75 |
| 435545 | AA687415 | Hs. 28107 | ESTs | 5.681E−03 | 4.72 | 50.75 |
| 445757 | AW449065 | Hs. 13264 | KIAA0856 protein | 2.299E−02 | 4.70 | 40.75 |
| 401593 | | | Target Exon | 2.299E−02 | 4.70 | 48.5 |
| 442552 | R20624 | | son of sevenless (Drosophila) homolog 1 | 3.476E−02 | 4.70 | 45.25 |
| 418660 | AW161979 | Hs. 100914 | hypothetical protein FLJ10352 | 5.132E−02 | 4.70 | 39.5 |
| 435368 | AI056230 | Hs. 117122 | ESTs | 5.766E−03 | 4.57 | 83.25 |
| 434253 | AI393345 | Hs. 116215 | ESTs | 5.091E−02 | 4.57 | 49.75 |
| 407578 | BE464417 | Hs. 131035 | ESTs, Weakly similar to CA24_HUMAN COLLA | 7.404E−02 | 4.57 | 48.25 |
| 424014 | AA333653 | Hs. 24790 | KIAA1573 protein | 9.366E−03 | 4.55 | 41 |
| 440798 | AB020648 | Hs. 7426 | KIAA0841 protein | 5.766E−03 | 4.52 | 44.25 |
| 426075 | AW513691 | Hs. 270149 | ESTs, Weakly similar to 2109260A B cell | 6.136E−02 | 4.52 | 43.25 |
| 455358 | AW902641 | | gb: QV3-NN1024-100500-181-d08 NN1024 Homo | 7.404E−02 | 4.50 | 41.75 |
| 449621 | AI656060 | Hs. 195726 | ESTs | 5.132E−02 | 4.47 | 44 |
| 459674 | AA180511 | | gb: zp53f03.r1 Stratagene NT2 neuronal pr | 7.404E−02 | 4.42 | 79 |
| 433085 | AA781270 | Hs. 220943 | ESTs | 3.444E−02 | 4.40 | 50.25 |
| 434995 | AW974995 | | gb: EST387100 MAGE resequences, MAGN Homo | 7.404E−02 | 4.40 | 40 |
| 456250 | U09196 | Hs. 82520 | polymerase (DNA-directed), delta 4 | 7.404E−02 | 4.38 | 44.75 |
| 425594 | AA649842 | Hs. 269334 | ESTs | 2.299E−02 | 4.38 | 34.75 |
| 402439 | | | C1002445*: gi\|4506787\|ref\|NP 003861.1\|IQ | 7.404E−02 | 4.37 | 43 |
| 429355 | AW973253 | Hs. 292689 | ESTs | 7.404E−02 | 4.35 | 46.75 |
| 453751 | R36762 | Hs. 101282 | Homo sapiens cDNA: FLJ21238 fis, clone C | 9.366E−03 | 4.32 | 63.75 |
| 444636 | T96667 | Hs. 17877 | ESTs | 5.132E−02 | 4.30 | 46.25 |
| 401197 | | | ENSP00000229263*: HSPC213. | 8.760E−02 | 4.27 | 39.25 |
| 432014 | H66741 | Hs. 38540 | ESTs, Weakly similar to ALU4_HUMAN ALU S | 7.404E−02 | 4.27 | 61.25 |
| 420556 | AA278300 | Hs. 124292 | Homo sapiens cDNA: FLJ23123 fis, clone L | 1.485E−02 | 4.25 | 59.75 |
| 413329 | AI056885 | Hs. 133539 | ESTs | 9.366E−03 | 4.25 | 40.25 |
| 427450 | AB014526 | Hs. 178121 | KIAA0626 gene product | 3.476E−02 | 4.24 | 41.25 |
| 431858 | AA732530 | Hs. 184019 | Homo sapiens clone 23551 mRNA sequence | 1.792E−02 | 4.22 | 45.75 |
| 408852 | AW291435 | Hs. 254961 | ESTs | 1.450E−02 | 4.22 | 39.5 |
| 433171 | AA579425 | | gb: nf37c08.s1 NCI_CGAP_Pr2 Homo sapiens | 3.476E−02 | 4.20 | 43.75 |
| 430920 | U96402 | Hs. 248132 | goosecoid-like | 7.404E−02 | 4.17 | 42.75 |
| 405206 | | | Target Exon | 8.705E−02 | 4.13 | 38.75 |
| 453815 | AL135365 | Hs. 126857 | Homo sapiens cDNA FLJ12936 fis. clone NT | 3.476E−02 | 4.07 | 44 |
| 440573 | BE550891 | Hs. 270624 | ESTs | 5.132E−02 | 4.05 | 32.25 |
| 411047 | AW938479 | | gb: CM0-DT0057-290200-253-h06 DT0057 Homo | 1.468E−02 | 4.04 | 39.5 |
| 400738 | | | Target Exon | 1.833E−02 | 4.00 | 36.5 |
| 423983 | AA333261 | | gb: EST37476 Embryo, 8 week 1 Homo sapien | 2.299E−02 | 4.00 | 32.5 |
| 403686 | | | C4001366*: gi\|9837427\|gb\|AAG00570.1\|AF287 | 1.485E−02 | 3.95 | 39.75 |
| 418677 | S83308 | Hs. 87224 | SRY (sex determining region Y)-box 5 | 1.468E−02 | 3.94 | 52.25 |
| 453861 | AI026838 | Hs. 30120 | ESTs, Weakly similar to NUCL_HUMAN NUCLE | 5.132E−02 | 3.93 | 33.75 |
| 401778 | AA463798 | | MCT-1 protein | 5.766E−03 | 3.90 | 40.75 |
| 448854 | AW245617 | Hs. 77703 | hypothetical protein FLJ11506 | 7.404E−02 | 3.89 | 32.5 |
| 456327 | H68741 | Hs. 38774 | ESTs | 3.476E−02 | 3.85 | 49.5 |
| 431300 | AA502346 | | gb: ne26b03.s1 NCI_CGAP_Co3 Homo sapiens | 5.091E−02 | 3.82 | 56.75 |
| 438146 | Z36842 | Hs. 57548 | ESTs | 2.299E−02 | 3.82 | 35 |
| 443222 | AW390067 | Hs. 132762 | ESTs | 2.275E−02 | 3.82 | 31.75 |
| 457247 | AA458605 | | KIAA1681 protein | 9.366E−03 | 3.82 | 91.5 |
| 406299 | | | Target Exon | 5.132E−02 | 3.80 | 30 |
| 430383 | AI861854 | Hs. 210778 | hypothetical protein FLJ10989 | 2.275E−02 | 3.77 | 49.75 |
| 453478 | AF083898 | Hs. 33021 | neuro-oncological ventral antigen 2 | 6.091E−02 | 3.77 | 32 |
| 439601 | AB029032 | Hs. 6606 | KIAA1109 protein | 7.404E−02 | 3.76 | 49.75 |
| 421828 | AW891965 | Hs. 279789 | histone deacetylase 3 | 3.476E−02 | 3.76 | 42.75 |
| 406528 | | | Target Exon | 3.476E−02 | 3.75 | 45.5 |
| 451614 | AA298812 | Hs. 98539 | ESTs | 7.353E−02 | 3.75 | 38.25 |
| 430320 | BE245290 | Hs. 239218 | uncharacterized hypothalamus protein HCD | 5.132E−02 | 3.75 | 36.5 |
| 441516 | F06700 | Hs. 7879 | interferon-related developmental regulat | 7.404E−02 | 3.73 | 30.75 |
| 430730 | T71087 | Hs. 44197 | hypothetical protein DKFZp564D0462 | 2.299E−02 | 3.72 | 44.25 |
| 430446 | AF131782 | Hs. 241438 | Homo sapiens clone 24941 mRNA sequence | 5.766E−03 | 3.72 | 46.25 |
| 414899 | AW975433 | Hs. 36288 | ESTs | 5.132E−02 | 3.69 | 33 |
| 447695 | AI420116 | Hs. 161269 | ESTs | 2.299E−02 | 3.69 | 30.25 |
| 402842 | | | ENSP00000241325*: DJ947L8.1.3 (novel CUB | 5.681E−03 | 3.65 | 30.5 |
| 446001 | AI827473 | Hs. 346435 | ESTs | 9.366E−03 | 3.63 | 46.25 |
| 402836 | | | ENSP00000251163*: Membrane-associated gua | 4.164E−02 | 3.60 | 57.25 |
| 410469 | AW749723 | Hs. 43586 | gb: QV3-BT0511-081299-035-d01 BT0511 Homo | 8.705E−02 | 3.60 | 37 |
| 421471 | U90545 | Hs. 327179 | solute carrier family 17 (sodium phospha | 3.476E−02 | 3.58 | 110.75 |
| 456962 | BE272095 | Hs. 167791 | reticulocalbin 1, EF-hand calcium bindin | 6.136E−02 | 3.57 | 35.75 |
| 400301 | X03635 | Hs. 1657 | estrogen receptor 1 | 1.485E−02 | 3.57 | 66.25 |
| 438099 | AA777767 | Hs. 187562 | ESTs | 4.200E−02 | 3.56 | 37.75 |
| 441243 | AI767056 | Hs. 193002 | ESTs | 2.299E−02 | 3.55 | 53 |
| 416401 | N80139 | Hs. 268916 | ESTs | 2.299E−02 | 3.55 | 49.75 |
| 402942 | | | Target Exon | 5.681E−03 | 3.55 | 35.5 |
| 451351 | AW058261 | Hs. 321435 | ESTs, Weakly similar to ALU1_HUMAN ALU S | 5.132E−02 | 3.53 | 38 |
| 457565 | BE294029 | Hs. 279903 | Ras homolog enriched in brain 2 | 2.299E−02 | 3.52 | 35 |
| 419287 | X91906 | Hs. 89872 | chloride channel 5 (nephrolithiasis 2, X | 9.366E−03 | 3.52 | 31.75 |
| 430807 | S74019 | Hs. 247979 | pre-B lymphocyte gene 1 | 2.807E−02 | 3.50 | 35.5 |
| 400302 | N48056 | | folate hydrolase (prostate-specific memb | 3.476E−02 | 3.48 | 63.75 |
| 404657 | | | C9000088*: gi\|6754878\|ref\|NP_035061.1\|nu | 1.468E−02 | 3.44 | 53.75 |
| 445664 | AW968638 | Hs. 237691 | ESTs, Weakly similar to KIAA0601-protein | 2.807E−02 | 3.42 | 39.5 |
| 436336 | AJ011378 | Hs. 186927 | ESTs | 4.056E−02 | 3.42 | 33.25 |
| 409959 | BE349470 | | mucin 6, gastric | 9.120E−03 | 3.40 | 47.5 |
| 458443 | AV647010 | Hs. 27 | glycine dehydrogenase (decarboxylating; | 4.200E−02 | 3.40 | 47.25 |
| 411214 | BE046571 | | gb: hn40g11.x1 NCI_CGAP_RDF2 Homo sapiens | 2.299E−02 | 3.38 | 39.25 |
| 437556 | AI223078 | Hs. 136776 | ESTs | 9.243E−03 | 3.35 | 36.25 |
| 425295 | AA431366 | Hs. 37251 | ESTs | 7.404E−02 | 3.32 | 50 |
| 409440 | AI239685 | Hs. 12315 | hypothetical protein FLJ11608 | 5.091E−02 | 3.32 | 30.75 |
| 405543 | | | C2002468*: gi\|3334856\|emb\|CAA11279.1\| (AJ | 4.970E−02 | 3.30 | 35.75 |
| 449986 | AW864502 | | gb: PM4-SN0016-120400-004-b12 SN0016 Homo | 2.299E−02 | 3.25 | 60.75 |
| 451790 | AA927403 | Hs. 3803 | reticulon 2 | 5.132E−02 | 3.24 | 41.5 |
| 456350 | BE246762 | Hs. 89499 | arachidonate 5-lipoxygenase | 5.132E−02 | 3.22 | 40.5 |
| 436169 | AA888311 | Hs. 17602 | Homo sapiens cDNA FLJ12381 fis, clone MA | 2.251E−02 | 3.22 | 33.25 |
| 421497 | BE145334 | Hs. 194637 | BANP homolog, SMAR1 homolog | 5.766E−03 | 3.22 | 30 |
| 449959 | AI076834 | Hs. 21160 | ESTs | 4.200E−02 | 3.20 | 35.25 |
| 441189 | AW450266 | Hs. 257276 | ESTs | 7.353E−02 | 3.19 | 31.75 |
| 411399 | AI144416 | Hs. 22248 | ESTs | 7.404E−02 | 3.15 | 31.75 |
| 447896 | AI436124 | Hs. 294069 | Homo sapiens cDNA FLJ13384 fis, clone PL | 2.299E−02 | 3.15 | 62 |
| 420728 | AA767718 | Hs. 93581 | hypothetical protein FLJ10512 | 5.132E−02 | 3.15 | 41.25 |
| 444959 | AV652120 | Hs. 213232 | ESTs, Weakly similar to 2004399A chromos | 1.485E−02 | 3.15 | 117 |
| 424049 | AB014524 | Hs. 138380 | KIAA0624 protein | 5.597E−03 | 3.13 | 61.25 |
| 444363 | AI142827 | Hs. 143656 | ESTs | 8.760E−02 | 3.12 | 35.5 |
| 437056 | AI147061 | | gb: ok33a11.s1 Soares_NSF_F8_9W_OT_PA_P_S | 5.091E−02 | 3.11 | 32.75 |
| 408931 | AA251995 | Hs. 334648 | poly(A) polymerase alpha | 2.299E−02 | 3.11 | 60 |
| 447135 | T58148 | | gb: yb98g06.s1 Stratagene lung (937210) H | 1.468E−02 | 3.10 | 34.75 |
| 451513 | AI800283 | Hs. 155787 | ESTs | 2.780E−02 | 3.08 | 31.75 |
| 430448 | AI633553 | Hs. 13303 | Homo sapiens cDNA: FLJ21784 fis, clone H | 5.010E−02 | 3.07 | 31.75 |
| 413922 | AI535895 | Hs. 221024 | ESTs | 3.476E−02 | 3.07 | 56 |
| 440700 | AW952281 | Hs. 296184 | guanine nucleotide binding protein (G pr | 1.485E−02 | 3.06 | 96.25 |
| 442049 | AA310393 | Hs. 190044 | ESTs | 7.404E−02 | 3.05 | 36.25 |
| 445159 | AI631036 | Hs. 196843 | ESTs | 5.132E−02 | 3.03 | 30.5 |
| 402781 | | | C1001326*: gi\|7292419\|gb\|AAF47823.1\|(AE0 | 3.444E−02 | 3.03 | 32.5 |
| 450127 | AI698001 | Hs. 200664 | ESTs | 1.833E−02 | 3.03 | 35.5 |
| 416336 | R97949 | Hs. 24128 | ESTs | 7.404E−02 | 3.02 | 42.5 |
| 428337 | AA644508 | | gb: af75C01.r1 Soares_NhHMPu_S1 Homo sapi | 2.275E−02 | 3.02 | 32.25 |
| 441959 | BE536998 | Hs. 200360 | Homo sapiens cDNA FLJ13027 fis. clone NT | 3.476E−02 | 3.01 | 34.25 |
| 450770 | AA019924 | Hs. 28803 | ESTs | 3.476E−02 | 3.01 | 52.75 |
| 414149 | BE313317 | Hs. 902 | neurofibromin 2 (bilateral acoustic neur | 7.404E−02 | 2.99 | 47.25 |
| 444622 | H77824 | | ESTs | 5.681E−03 | 2.98 | 51 |
| 444909 | AI933051 | Hs. 192280 | ESTs | 5.132E−02 | 2.97 | 42.75 |
| 419662 | W87674 | | gb: zh66a01.r1 Soares_fetal_liver_spleen_{—} | 7.166E−03 | 2.97 | 36 |
| 439435 | AI627654 | Hs. 174497 | ESTs, Weakly similar to S43529 165 K prot | 6.091E−02 | 2.97 | 36.5 |
| 435902 | AA701867 | Hs. 297726 | ESTs | 7.404E−02 | 2.95 | 70 |
| 406526 | | | C19001782*: gi\|8922411\|ref\|NP_060558.1\|h | 5.766E−03 | 2.95 | 51.5 |
| 405846 | | | Target Exon | 5.132E−02 | 2.95 | 39.75 |
| 448920 | AW408009 | Hs. 22580 | alkylglycerone phosphate synthase | 4.164E−02 | 2.92 | 43 |
| 422561 | AA991499 | Hs. 118354 | CAT56 protein | 5.132E−02 | 2.92 | 42.75 |
| 440915 | AI809240 | Hs. 210383 | olfactory receptor, family 4, subfamily | 1.485E−02 | 2.92 | 31.25 |
| 449495 | AI652833 | | gb: wb22c11.x1 NCI_CGAP_GC6 Homo sapiens | 5.132E−02 | 2.90 | 34.75 |
| 411426 | BE141714 | | gb: QV0-HT0101-061099-032-c04 HT0101 Homo | 4.200E−02 | 2.90 | 33 |
| 435236 | T03890 | Hs. 157208 | ESTs, Highly similar to ARX MOUSE HOMEOB | 7.268E−03 | 2.88 | 44.75 |
| 405152 | | | Target Exon | 7.353E−02 | 2.88 | 37.5 |
| 457230 | T73510 | Hs. 209153 | angiopoietin-like 3 | 1.468E−02 | 2.82 | 50 |
| 455485 | AA102287 | Hs. 26756 | hypothetical protein FLJ20896 | 5.132E−02 | 2.82 | 46.5 |
| 452266 | AI767250 | Hs. 165240 | ESTs | 3.476E−02 | 2.81 | 49 |
| 418154 | BE165866 | | nuclear receptor subfamily 1, group I, m | 7.404E−02 | 2.80 | 65.75 |
| 405481 | | | Target Exon | 5.132E−02 | 2.80 | 46.5 |
| 404162 | | | NM_022113*: Homo sapiens kinesin family m | 3.476E−02 | 2.80 | 40.5 |
| 415647 | F13458 | Hs. 6985 | ESTs | 7.353E−02 | 2.80 | 40 |
| 456505 | AA504595 | Hs. 111418 | ESTs | 1.168E−02 | 2.78 | 52.5 |
| 447567 | AW474513 | Hs. 224397 | ESTs, Weakly similar to I38931 Wiskott-A | 5.766E−03 | 2.77 | 43 |
| 422473 | U94780 | Hs. 117242 | meningioma expressed antigen 6 (coiled-c | 7.353E−02 | 2.77 | 40.25 |
| 452830 | AW976032 | Hs. 288912 | hypothetical protein FLJ22604 | 3.476E−02 | 2.76 | 30 |
| 428375 | T65153 | Hs. 260282 | ESTs | 6.091E−02 | 2.76 | 32.5 |
| 444618 | AV653785 | Hs. 173334 | ELL-RELATED RNA POLYMERASE II, ELONGATIO | 7.268E−03 | 2.73 | 30.25 |
| 432629 | AW860548 | Hs. 280658 | ESTs | 2.807E−02 | 2.73 | 55.25 |
| 457747 | AW975000 | | gb: EST387105 MAGE resequences, MAGN Homo | 9.366E−03 | 2.71 | 48 |
| 449210 | AI635363 | Hs. 345517 | ESTs | 7.404E−02 | 2.71 | 30 |
| 419711 | C02621 | Hs. 159282 | ESTs | 2.251E−02 | 2.71 | 32 |
| 407609 | R43159 | Hs. 238432 | ESTs | 5.132E−02 | 2.70 | 47.25 |
| 438850 | R33727 | Hs. 24688 | EST | 1.468E−02 | 2.70 | 37.75 |
| 449000 | U69560 | Hs. 3826 | kelch-like protein C3IP1 | 3.444E−02 | 2.70 | 33.5 |
| 413359 | AA524285 | Hs. 154172 | ESTs, Moderately similar to BCGF_HUMAN B | 7.404E−02 | 2.67 | 34.75 |
| 402168 | | | NM_022046*: Homo sapiens kallikrein 14 (K | 5.132E−02 | 2.66 | 65 |
| 423665 | BE167153 | Hs. 24380 | ESTs | 5.132E−02 | 2.65 | 47.75 |
| 409872 | AW502313 | | gb: UI-HF-BR0p-ajs-d-08-0-Ul.r1 NIH_MGC_5 | 7.404E−02 | 2.65 | 45.5 |
| 419853 | AA252006 | Hs. 190150 | ESTs | 7.404E−02 | 2.65 | 32.25 |
| 409277 | T05558 | Hs. 156880 | ESTs | 7.404E−02 | 2.65 | 59.75 |
| 401114 | | | Target Exon | 2.299E−02 | 2.64 | 33.25 |
| 433847 | AA610266 | | ESTs | 2.299E−02 | 2.64 | 42.25 |
| 412204 | AI125507 | Hs. 130829 | ESTs | 5.132E−02 | 2.63 | 37 |
| 454302 | AA306105 | Hs. 50785 | SEC22, vesicle trafficking protein (S. c | 6.136E−02 | 2.63 | 33.5 |
| 425744 | AA362985 | | gb: EST72769 Ovary II Homo sapiens cDNA 5 | 3.476E−02 | 2.61 | 43 |
| 404265 | | | ENSP00000243218*: DJ329L24.3 (member of M | 1.485E−02 | 2.60 | 48.25 |
| 447113 | AA741545 | Hs. 282832 | ESTs, Weakly similar to T24961 hypotheti | 3.476E−02 | 2.60 | 36.75 |
| 418383 | AA218986 | Hs. 118854 | ESTs | 5.132E−02 | 2.60 | 31.5 |
| 448339 | AL035920 | Hs. 20938 | RNA binding motif, single stranded inter | 3.476E−02 | 2.60 | 33.5 |
| 435321 | R16814 | Hs. 112062 | ESTs | 1.485E−02 | 2.59 | 147.5 |
| 451898 | T92572 | Hs. 142019 | ESTs, Weakly similar to 1207289A reverse | 3.476E−02 | 2.59 | 32.25 |
| 436869 | NM_014867 | Hs. 5333 | KIAA0711 gene product | 1.168E−02 | 2.59 | 48.5 |
| 450016 | AA249590 | Hs. 100748 | ESTs, Weakly similar to A28996 proline-r | 2.299E−02 | 2.57 | 54.25 |
| 434421 | AI915927 | Hs. 34771 | ESTs | 7.353E−02 | 2.57 | 73.25 |
| 446783 | AW138343 | Hs. 141867 | ESTs | 1.485E−02 | 2.56 | 58 |
| 418812 | AB018256 | Hs. 283881 | KIAA0713 protein | 5.132E−02 | 2.55 | 34.5 |
| 417157 | N49713 | | gb: yv23f06.s1 Soares fetal liver spleen | 8.760E−02 | 2.54 | 32.75 |
| 400308 | AF041410 | | Target | 2.275E−02 | 2.53 | 34.5 |
| 431082 | AA491600 | | gb: ne80a11.s1 NCI_CGAP_Ew1 Homo sapiens | 5.132E−02 | 2.52 | 66.75 |
| 451850 | R14553 | Hs. 301663 | ESTs | 6.136E−02 | 2.51 | 31.75 |
| 406029 | | | Target Exon | 7.404E−02 | 2.50 | 46.25 |
| 413471 | BE142098 | | gb: CM4-HT0137-220999-017-d11 HT0137 Homo | 3.444E−02 | 2.50 | 44.5 |
| 405014 | AF042838 | | mitogen-activated protein kinase kinase | 7.353E−02 | 2.50 | 35.25 |
| 432634 | AI650267 | Hs. 196169 | ESTs | 5.132E−02 | 2.50 | 32.75 |
| 433187 | R53995 | Hs. 293381 | ESTs, Moderately similar to ALU7_HUMAN A | 5.132E−02 | 2.47 | 33.5 |
| 443832 | AI829610 | Hs. 23531 | ESTs | 3.476E−02 | 2.47 | 37.5 |
| 447371 | AA334274 | Hs. 18368 | DKFZP564B0769 protein | 7.404E−02 | 2.46 | 44.75 |
| 458455 | AV648310 | Hs. 213488 | ESTs | 3.444E−02 | 2.45 | 58.5 |
| 447980 | AI703397 | Hs. 202355 | ESTs | 8.705E−02 | 2.45 | 33 |
| 419511 | AA429750 | Hs. 75113 | general transcription factor IIIA | 1.485E−02 | 2.45 | 41.25 |
| 412510 | AI056689 | Hs. 133538 | ESTs, Weakly similar to ALU1_HUMAN ALU S | 5.681E−03 | 2.45 | 46.25 |
| 416153 | R13894 | | gb: yf62a06.r1 Soares infant brain 1NIB H | 1.468E−02 | 2.44 | 42.75 |
| 458492 | AI143655 | Hs. 231200 | ESTs | 1.168E−02 | 2.43 | 47.5 |
| 425224 | AA352856 | Hs. 147211 | ESTs | 7.404E−02 | 2.40 | 44.75 |
| 413489 | BE144228 | | gb: MR0-HT0165-140200-009-d04 HT0165 Homo | 1.168E−02 | 2.40 | 36.75 |
| 437114 | AA836641 | Hs. 163085 | ESTs | 1.485E−02 | 2.38 | 53.5 |
| 457558 | AF083955 | Hs. 279852 | G protein-coupled receptor | 5.132E−02 | 2.37 | 45 |
| 429886 | AL050145 | Hs. 225986 | Homo sapiens mRNA; cDNA DKFZp586C2020 (f | 1.168E−02 | 2.35 | 52.25 |
| 403171 | | | C2001472*: gi\|5809678\|gb\|AAB41848.2\|(U64 | 3.476E−02 | 2.35 | 50.5 |
| 406126 | | | ENSP00000246871: PMS6 PROTEIN (HPMS6 PROT | 3.476E−02 | 2.35 | 47.25 |
| 428673 | AW601325 | Hs. 337757 | Homo sapiens mRNA; cDNA DKFZp566M063 (fr | 5.132E−02 | 2.35 | 37.75 |
| 421466 | U88063 | Hs. 104633 | agouti (mouse) related protein | 7.404E−02 | 2.33 | 37.5 |
| 453285 | X99894 | Hs. 32938 | insulin promoter factor 1, homeodomain t | 7.404E−02 | 2.32 | 36.5 |
| 441927 | AW850555 | Hs. 39925 | ESTs, Weakly similar to A46010 X-linked | 7.404E−02 | 2.32 | 30.75 |
| 443368 | BE568891 | Hs. 199210 | ESTs, Moderately similar to bK116F5.2 [H | 5.681E−03 | 2.32 | 42.25 |
| 430517 | S80071 | Hs. 241597 | solute carrier family 6 (neurotransmitte | 2.299E−02 | 2.31 | 45.5 |
| 450734 | AI732317 | Hs. 299119 | ESTs, Moderately similar to 2211404A B21 | 7.353E−02 | 2.30 | 34.5 |
| 406466 | | | Target Exon | 9.243E−03 | 2.30 | 48.75 |
| 431025 | AA490842 | Hs. 105269 | ESTs | 2.807E−02 | 2.30 | 38.5 |
| 421573 | AI302850 | Hs. 262455 | ESTs | 4.200E−02 | 2.29 | 44.75 |
| 429031 | BE002237 | Hs. 239666 | Homo sapiens cDNA FLJ13495 fis, clone PL | 3.476E−02 | 2.28 | 42.25 |
| 403545 | | | Target Exon | 1.812E−02 | 2.27 | 40.5 |
| 418063 | R38973 | Hs. 329841 | EST | 2.299E−02 | 2.27 | 39 |
| 412243 | W37901 | Hs. 278349 | ESTs | 3.444E−02 | 2.27 | 31 |
| 447793 | AI424924 | Hs. 211203 | ESTs | 3.476E−02 | 2.26 | 42.25 |
| 402370 | | | Target Exon | 1.485E−02 | 2.25 | 58.25 |
| 404150 | | | Target Exon | 7.404E−02 | 2.25 | 53.75 |
| 431668 | AW969610 | Hs. 151179 | ESTs | 5.132E−02 | 2.25 | 37.75 |
| 442174 | AI690080 | Hs. 128907 | ESTs, Weakly similar to ARIX homeodomain | 7.404E−02 | 2.24 | 44.75 |
| 444758 | AL044878 | Hs. 11899 | 3-hydroxy-3-methylglutaryl-Coenzyme A re | 5.132E−02 | 2.24 | 45 |
| 431395 | AW752337 | Hs. 193666 | haspin | 1.468E−02 | 2.24 | 36 |
| 447115 | AI815852 | Hs. 205865 | ESTs, Weakly similar to S02392 alpha-2-m | 3.444E−02 | 2.24 | 34.75 |
| 408847 | AW290997 | Hs. 30348 | ESTs | 2.275E−02 | 2.22 | 46 |
| 420843 | H96982 | Hs. 42321 | ESTs | 7.353E−02 | 2.22 | 39.25 |
| 423732 | AF058056 | Hs. 132183 | solute carrier family 16 (monocarboxylic | 7.404E−02 | 2.21 | 51.75 |
| 447083 | AI472124 | Hs. 157757 | ESTs | 5.766E−03 | 2.21 | 46.5 |
| 425757 | AA363171 | | gb: EST72986 Ovary II Homo sapiens cDNA 5 | 3.476E−02 | 2.20 | 33.25 |
| 401941 | | | Target Exon | 3.476E−02 | 2.19 | 39 |
| 421193 | D60983 | Hs. 5096 | ESTs, Weakly similar to I38022 hypotheti | 1.450E−02 | 2.19 | 42.25 |
| 441357 | AI240184 | Hs. 126819 | ESTs | 7.404E−02 | 2.19 | 33.5 |
| 451068 | AW294432 | Hs. 144252 | ESTs | 7.404E−02 | 2.18 | 32.25 |
| 427332 | R09418 | Hs. 261101 | ESTs, Weakly similar to I38022 hypotheti | 8.760E−02 | 2.18 | 45.5 |
| 401929 | | | C17001690: gi\|6005701\|ref\|NP_009099.1\|AT | 5.091E−02 | 2.18 | 96.5 |
| 433444 | AW975324 | Hs. 129816 | ESTs | 3.476E−02 | 2.18 | 94.25 |
| 444331 | AW193342 | Hs. 24144 | ESTs | 7.353E−02 | 2.18 | 43.25 |
| 445828 | F05802 | Hs. 81907 | ESTs | 1.485E−02 | 2.17 | 64.25 |
| 452137 | AI861840 | Hs. 211687 | ESTs | 5.132E−02 | 2.17 | 34.75 |
| 409061 | AI204994 | Hs. 7874 | Homo sapiens cDNA: FLJ21435 fis, clone C | 2.299E−02 | 2.16 | 31 |
| 457281 | BE253012 | Hs. 153400 | ESTs, Weakly similar to ALU1_HUMAN ALU S | 7.404E−02 | 2.16 | 31.25 |
| 426129 | NM_000348 | Hs. 1989 | steroid-5-alpha-reductase, alpha polypep | 3.444E−02 | 2.15 | 40.75 |
| 453894 | AW937825 | Hs. 56847 | Homo sapiens cDNA FLJ12874 fis, clone NT | 6.136E−02 | 2.14 | 54.25 |
| 403923 | | | Target Exon | 3.444E−02 | 2.13 | 49.25 |
| 428655 | H05769 | Hs. 188757 | Homo sapiens, clone MGC: 5564, mRNA, comp | 2.299E−02 | 2.13 | 32.5 |
| 454333 | AW373209 | | gb: RC5-BT0506-271199-031-C12 BT0506 Homo | 3.444E−02 | 2.12 | 44.5 |
| 445805 | C16975 | Hs. 301444 | KIAA1673 | 4.200E−02 | 2.11 | 32 |
| 446709 | R10490 | Hs. 156341 | ESTs | 5.431E−03 | 2.10 | 32.25 |
| 436505 | AJ277841 | Hs. 120963 | ELG protein | 7.404E−02 | 2.10 | 41.75 |
| 427761 | AA412205 | Hs. 140996 | ESTs | 4.929E−02 | 2.10 | 31.75 |
| 417720 | AA205625 | Hs. 208067 | ESTs | 5.766E−03 | 2.10 | 63.5 |
| 417688 | R09170 | Hs. 284350 | ESTs | 1.485E−02 | 2.10 | 36.25 |
| 434521 | NM_002267 | Hs. 3886 | karyopherin alpha 3 (importin alpha 4) | 7.353E−02 | 2.09 | 35 |
| 444900 | AI360120 | Hs. 148581 | ESTs | 5.132E−02 | 2.06 | 34.5 |
| 444595 | AL121094 | Hs. 83572 | hypothetical protein MGC14433 | 3.444E−02 | 2.05 | 34 |
| 406180 | AB018249 | | small inducible cytokine subfamily A (Cy | 7.404E−02 | 2.05 | 45.25 |
| 423378 | BE313601 | Hs. 164866 | hypothetical protein FLJ22558 | 3.476E−02 | 2.04 | 41 |
| 427227 | AF103803 | Hs. 283690 | hypothetical protein | 6.136E−02 | 2.03 | 36.25 |
| 438163 | AI056258 | Hs. 122523 | ESTs | 6.091E−02 | 2.02 | 55.75 |
| 429219 | AI221480 | Hs. 99161 | ESTs | 5.132E−02 | 2.02 | 39.25 |
| 438011 | BE466173 | Hs. 145696 | splicing factor (CC1.3) | 1.485E−02 | 2.02 | 38.25 |

Pkey: Unique Eos probeset identifier number
ExAccn: Exemplar Accession number, Genbank accession number
UnigeneID: Unigene number
Unigene Title: Unigene gene title
R1: Wilcoxon rank-sum test p-value
R2: Trimean of responders over Trimean of non-responders Ratio
R3: Difference of Trimean of responders vs. Trimean of non-responders

TABLE 6B

| Pkey | CAT Number | Accession |
|---|---|---|
| 409872 | 1156673_1 | AW502313 AW502681 AW502682 |
| 409959 | 1162663_1 | BE349470 BE179199 BE179195 BE179198 BE179204 BE162686 AW513804 |
| 411047 | 1230266_1 | AW938479 AW850678 AW814826 |
| 411214 | 1235915_1 | BE046571 BE046734 BE046555 BE046651 AW827568 |
| 411426 | 1245515_1 | BE141714 AW845993 AW845989 |
| 413471 | 1371778_1 | BE142098 BE142092 |
| 413489 | 1373392_1 | BE144228 BE144291 |
| 416153 | 1573947_1 | R13894 H23037 R56371 |
| 417157 | 1653833_1 | N49713 N49819 W03810 |
| 418154 | 17249_1 | BE165866 BE165832 AA319621 AA401166 AI811901 H78857 X56199 R93797 |
| | | AW896675 AA401072 AW374411 H52942 AW896685 AA348138 |
| | | AI399764 AA010244 W90159 N90874 AA339496 AW967136 W38705 AA029093 |
| | | AW444647 BE175700 AV651656 AV651847 AA332039 |
| | | AV649227 AV649164 AV649491 N87956 AA332262 BE001561 H75493 |
| | | BE218742 AA333298 AA095633 AA091968 M78602 T05342 W17094 |
| | | AA126501 AW374665 AI452905 AW316900 AI185080 AI202928 AI651843 AA693541 |
| | | AI681019 AV658257 AV658133 BE045335 BE089546 |
| | | AA300830 AA361376 BE218739 AW207622 AA765340 AW612733 BE348741 AI806054 |
| | | AI871563 AA808652 AI500693 AW342032 AA147066 |
| 419662 | 1870052_1 | W87674 W87872 W87774 |
| 423983 | 233891_1 | AA333261 AA333365 AA552870 |
| 425744 | 255834_1 | AA362985 AW963337 T27244 |
| 425757 | 255956_1 | AA363171 AW963347 AA371863 |
| 428337 | 289967_1 | AA644508 AA479489 AA426174 |
| 431082 | 327710_1 | AA491600 AA491645 AI920986 |
| 431300 | 331217_1 | AA502346 BE159863 |
| 432474 | 348197_1 | AA584042 AW973273 AA548798 |
| 433171 | 360292_1 | AA579425 AW969965 AA579102 |
| 433847 | 374914_1 | AA610266 AA610273 AA632625 AA812563 AI688018 AI094802 H79160 R99139 |
| 434995 | 397210_1 | AW974995 AI821880 AI821932 AI791196 AA659617 AI821137 AA658925 |
| 437056 | 432262_1 | AI147061 AA743380 AA765223 AW976398 AI803927 |
| 442552 | 54472_3 | R20624 AA809852 AW025682 AW292949 H02596 AA633530 AA846566 AA806021 |
| | | AA713679 AI364631 BE350976 AW076131 AW518885 |
| | | AW263078 AI417702 AW086132 AI628605 AI769772 H02691 AW591478 H50040 AA227699 |
| | | AI885558 AA400937 AI261878 R82844 AW075473 |
| | | AI453800 N56889 AI922512 N51071 N51720 N51823 A1654491 W95290 AI272033 AI695006 |
| | | AI918702 AI638383 BE552047 AI783684 AI126790 |
| | | H01465 BE243273 |
| 444622 | 61286_1 | H77824 AA370099 AW956039 H73387 R93651 AV650472 |
| 447135 | 70963_1 | T58148 AW516579 AW059603 |
| 449495 | 808345_1 | AI652833 AI695904 AW888916 |
| 449625 | 8113_1 | NM_014253 AF100772 BE088769 AL022718 BE161779 AW863569 BE161640 AL039060 |
| | | BE168542 AW296554 AA323193 AA235370 AW779760 |
| | | N48674 AI375997 R45432 D59344 AI203107 F07491 R35360 R25094 AI913631 AI498402 T61382 |
| | | AI016320 N45526 T61415 AA331486 |
| 449986 | 821463_1 | AW864502 AW864369 AI678780 |
| 454333 | 1115507_1 | AW373209 AW373205 |
| 455358 | 1284494_1 | AW902641 AW902569 AW902654 AW902557 AW902650 AW902644 AW902741 |
| 457247 | 308656_1 | AA458605 AW977252 AI261627 AW274550 AI418272 AW665579 |
| | | AA731376 AW293861 D80453 AI217860 |
| 457747 | 397222_1 | AW975000 AA658945 AA661558 |

Pkey: Unique Eos probeset identifier number
CAT number: Gene cluster number
Accession: Genbank accession numbers

TABLE 6C

| Pkey | Ref | Strand | Nt_position |
|---|---|---|---|
| 400738 | 7321559 | Plus | 150214-150446 |
| 400864 | 9798617 | Minus | 2555-2673, 27827-28000 |
| 401114 | 9966554 | Plus | 52327-53385 |
| 401197 | 9719705 | Plus | 176341-176452 |
| 401529 | 7770649 | Minus | 14577-15668 |
| 401593 | 7230957 | Plus | 10368-10572, 11293-12356 |
| 401757 | 7239630 | Plus | 88641-88751 |
| 401778 | 7249133 | Minus | 136881-137147, 137499-137628, |
| | | | 137858-138143, 141015-142602 |
| 401929 | 3810670 | Minus | 3167-3286, 4216-4310 |
| 401941 | 4982556 | Plus | 112022-112204 |
| 402168 | 7458725 | Minus | 43245-43397 |
| 402370 | 9558580 | Minus | 14525-15733 |
| 402439 | 9796503 | Minus | 108604-108764 |
| 402781 | 7387389 | Plus | 100409-100666 |
| 402836 | 8745058 | Minus | 96756-96941 |
| 402842 | 9369121 | Minus | 76355-76479 |
| 402942 | 9368398 | Plus | 102152-102386 |
| 403171 | 9838164 | Minus | 74502-74703 |
| 403545 | 8078400 | Plus | 25293-25640 |
| 403609 | 8308266 | Minus | 125974-126320 |
| 403686 | 7387348 | Minus | 66625-68364 |
| 403923 | 7454203 | Minus | 1793-2128 |
| 404150 | 7534008 | Plus | 165811-165943 |
| 404162 | 9926427 | Minus | 107973-108135 |
| 404265 | 9437317 | Minus | 50752-50877 |
| 404657 | 9797066 | Minus | 12444-12548, 17348-17455 |
| 405014 | 6478993 | Minus | 100709-100845 |
| 405152 | 9965561 | Minus | 137662-137969 |
| 405206 | 6692345 | Plus | 17807-18338, 20430-20538 |
| 405481 | 3688109 | Plus | 5718-5837, 8719-8818 |
| 405543 | 9857582 | Minus | 104338-104449 |
| 405846 | 7637314 | Plus | 6623-7188 |
| 406029 | 8312328 | Plus | 63187-63801 |
| 406126 | 7108191 | Minus | 46035-46534 |
| 406180 | 7283201 | Minus | 38923-39107 |
| 406299 | 5686278 | Minus | 35655-36119 |
| 406466 | 9795550 | Plus | 115511-115658, 118766-118915, |
| | | | 121699-121771 |
| 406526 | 7711448 | Minus | 102742-102944 |
| 406528 | 7711464 | Minus | 11201-11407, 12589-12733, |
| | | | 13287-13499 |

Pkey: Unique number corresponding to an Eos probeset
Ref: Sequence source. The 7 digit numbers in this column are Genbank Identifier (GI) numbers. “Dunham I. et al.” refers to the publication entitled “The DNA sequence of human chromosome 22” Dunham, et al. (1999) Nature 402: 489-495.
Strand: Indicates DNA strand from which exons were predicted.
Nt_position: Indicates nucleotide positions of predicted exons.

Table 7A lists about 1298 genes up-regulated in Hepatitis C positive liver tissues compared to Hepatitis C negative liver tissues. These genes have potential to be diagnostics and/or prognostic markers for Hepatitis C infected liver tissues. They may also provide clinical information on Hepatitis C infection and pathology. They may also be potential targets for therapeutic drugs and/or treatments. These were selected from 59680 probesets on the Affymetrix/Eos Hu03 GeneChip array such that the Wilcoxon rank-sum test p-value between the 2 groups was less than 0.10, the ratio of the “weighted average” of Hepatitis C positive liver tissues to the “weighted average” of Hepatitis C negative liver tissues was equal to or above 2.0, and that the differences between the same 2 groups was equal to or above 30.0. The “weighted average” of the Hepatitis C positive liver tissues was set to the trimean of several different Hepatitis C positive liver tissues. The “weighted average” of the Hepatitis C negative liver tissues was set to the either 10 or the trimean of several different Hepatitis C negative liver tissues, whichever value was greater to eliminate ratios with a denominator of zero or less.

TABLE 7A

| Pkey | ExAccn | UniGene | Unigene Title | R1 | R2 |
|---|---|---|---|---|---|
| 428227 | AA321649 | Hs. 2248 | small inducible cytokine subfamily B (Cy | 1.74E−07 | 25.27 |
| 414004 | M737033 | Hs. 7155 | ESTs, Moderately similar to 2115357A TYK | 3.18E−07 | 24.52 |
| 448111 | AA053486 | Hs. 20315 | interferon-induced protein with tetratri | 4.93E−07 | 24.00 |
| 417621 | AV654694 | Hs. 82316 | interferon-induced, hepatitis C-associat | 2.36E−07 | 23.13 |
| 422746 | NM_004484 | Hs. 119651 | glypican 3 | 4.03E−06 | 22.55 |
| 426711 | AA383471 | Hs. 343800 | conserved gene amplified in osteosarcoma | 1.75E−07 | 20.97 |
| 424090 | X99699 | Hs. 139262 | XIAP associated factor-1 | 2.78E−05 | 17.80 |
| 433854 | AA610649 | Hs. 333239 | ESTs | 2.26E−04 | 15.90 |
| 451652 | AA018968 | Hs. 133536 | ESTs | 1.62E−04 | 15.05 |
| 427283 | AL119796 | Hs. 174185 | ectonucleotide pyrophosphatase/phosphodi | 4.02E−06 | 14.90 |
| 413670 | AB000115 | Hs. 75470 | hypothetical protein, expressed in osteo | 6.13E−07 | 14.59 |
| 418216 | AA662240 | Hs. 283099 | AF15q14 protein | 4.93E−07 | 14.17 |
| 425787 | AA363867 | Hs. 155029 | ESTs | 7.26E−05 | 13.57 |
| 446094 | AK001760 | Hs. 13801 | KIAA1685 protein | 2.95E−05 | 12.90 |
| 450937 | R49131 | Hs. 26267 | ATP-dependant interferon response protei | 4.61E−06 | 12.07 |
| 442048 | M974603 | | gb: op34f05.s1 Soares_NFL_T_GBC_S1 Homo s | 3.34E−05 | 11.92 |
| 408393 | AW015318 | Hs. 23165 | ESTs | 1.31E−05 | 11.82 |
| 415443 | T07353 | Hs. 7948 | ESTs | 1.67E−03 | 11.80 |
| 417308 | H60720 | Hs. 81892 | KIAA0101 gene product | 2.17E−05 | 11.70 |
| 416206 | AW206248 | Hs. 111092 | hypothetical protein FLJ22332 | 2.50E−06 | 11.35 |
| 432378 | AI493046 | Hs. 146133 | ESTs | 3.52E−06 | 11.13 |
| 430200 | BE613337 | Hs. 234896 | geminin | 7.59E−07 | 11.04 |
| 445823 | AI478563 | Hs. 145519 | FKSG87 protein | 1.15E−05 | 11.00 |
| 447973 | AB011169 | Hs. 20141 | similar to S. cerevisiae SSM4 | 4.52E−05 | 10.97 |
| 409153 | W03754 | Hs. 50813 | hypothetical protein FLJ20022 | 5.70E−07 | 10.72 |
| 451752 | AB032997 | | KIAA1171 protein | 1.74E−07 | 10.54 |
| 414052 | AW578849 | Hs. 283552 | ESTs, Weakly similar to unnamed protein | 1.77E−06 | 10.45 |
| 409231 | AA446644 | Hs. 692 | GA733-2 antigen; epithelial glycoprotein | 1.03E−04 | 10.30 |
| 432094 | AI658580 | Hs. 61426 | Homo sapiens mesenchymal stem cell prote | 5.74E−05 | 10.17 |
| 403790 | | | NM_001334*: Homo sapiens cathepsin O (CTS | 1.08E−03 | 9.65 |
| 421057 | T58283 | | Homo sapiens cDNA: FLJ22063 fis, clone H | 4.57E−03 | 9.27 |
| 428065 | AI634046 | Hs. 157313 | ESTs | 5.43E−03 | 9.22 |
| 444665 | BE613126 | Hs. 47783 | B aggressive lymphoma gene | 4.58E−06 | 9.17 |
| 435812 | AA700439 | Hs. 188490 | ESTs | 5.03E−04 | 9.13 |
| 418143 | M283057 | Hs. 266957 | hypothetical protein FLJ14281 | 5.30E−04 | 9.00 |
| 435665 | AI248952 | Hs. 12320 | ESTs | 1.77E−06 | 8.85 |
| 407644 | D16815 | | nuclear receptor subfamily 1, group D, m | 4.31E−04 | 8.85 |
| 421282 | AA286914 | Hs. 40782 | ESTs | 4.54E−04 | 8.67 |
| 422546 | AB007969 | Hs. 301478 | KIAA0500 protein | 4.00E−05 | 8.52 |
| 409512 | AW979187 | Hs. 293591 | melanoma differentiation associated prot | 2.36E−07 | 8.50 |
| 429490 | AI971131 | Hs. 23889 | ESTs, Weakly similar to ALU7 HUMAN ALU S | 1.15E−04 | 8.50 |
| 442994 | AI026718 | Hs. 16954 | ESTs | 2.43E−03 | 8.47 |
| 416224 | NM_002902 | Hs. 79088 | reticulocalbin 2, EF-hand calcium bindin | 3.74E−06 | 8.45 |
| 417933 | X02308 | Hs. 82962 | thymidylate synthetase | 2.02E−03 | 8.35 |
| 405102 | | | C15001220*: gi\|4469558\|gb\|AAD21311.1\| (AF | 1.93E−03 | 8.20 |
| 444314 | AI140497 | | gb: ow76b09.s1 Soares_fetal liver_spleen_{—} | 1.38E−03 | 8.10 |
| 426818 | AA554827 | Hs. 292996 | DKFZp434A0131 protein | 9.75E−03 | 8.02 |
| 432954 | AI076345 | | ESTs | 2.02E−03 | 7.88 |
| 449541 | AA730673 | Hs. 188634 | ESTs | 7.22E−04 | 7.85 |
| 410315 | AI638871 | Hs. 152519 | Homo sapiens cDNA: FLJ22524 fis. clone H | 1.30E−05 | 7.80 |
| 433586 | T85301 | | gb: yd78d06.s1 Soares fetal liver spleen | 3.20E−03 | 7.80 |
| 436995 | AI160015 | Hs. 125489 | ESTs | 5.89E−04 | 7.80 |
| 446619 | AU076643 | Hs. 313 | secreted phosphoprotein 1 (osteopontin, | 4.00E−03 | 7.80 |
| 407949 | W21874 | Hs. 247057 | ESTs, Weakly similar to 2109260A B cell | 1.03E−04 | 7.72 |
| 421904 | BE143533 | Hs. 109309 | hypothetical protein FLJ20035 | 7.61E−07 | 7.67 |
| 424848 | AI263231 | Hs. 327090 | EST | 2.11E−03 | 7.63 |
| 433401 | AF039698 | Hs. 284217 | serologically defined colon cancer antig | 1.57E−05 | 7.55 |
| 435571 | AF212225 | Hs. 283693 | mitochondrial ribosomal protein L1 | 8.63E−05 | 7.55 |
| 419175 | AW270037 | | KIAA0779 protein | 1.01E−05 | 7.50 |
| 407930 | AA045847 | Hs. 188361 | Homo sapiens cDNA FLJ12807 fis, clone NT | 6.86E−04 | 7.47 |
| 400247 | | | Eos Control | 8.61E−03 | 7.38 |
| 449935 | AA004798 | Hs. 108311 | ESTs, Weakly similar to T00351 hypotheti | 1.80E−05 | 7.38 |
| 435102 | AW899053 | Hs. 76917 | F-box only protein 8 | 3.50E−03 | 7.32 |
| 418452 | BE379749 | Hs. 85201 | C-type (calcium dependent, carbohydrate- | 4.25E−07 | 7.32 |
| 425053 | AF046024 | Hs. 154320 | ubiquitin-activating enzyme E1C (homolog | 2.81E−04 | 7.30 |
| 431183 | NM_006855 | Hs. 250696 | KDEL (Lys-Asp-Glu-Leu) endoplasmic retic | 1.10E−02 | 7.30 |
| 446488 | AB037782 | Hs. 15119 | KIAA1361 protein | 2.12E−03 | 7.25 |
| 458725 | AW970192 | Hs. 171942 | ras responsive element binding protein 1 | 1.71E−04 | 7.25 |
| 449718 | AA459480 | Hs. 23956 | hypothetical protein FLJ20502 | 1.80E−05 | 7.17 |
| 418840 | AI821614 | Hs. 185831 | ESTs | 1.63E−02 | 7.10 |
| 408063 | BE086548 | Hs. 42346 | calcineurin-binding protein calsarcin-1 | 1.71E−04 | 7.05 |
| 436024 | AI800041 | | ESTs | 2.81E−04 | 7.02 |
| 402727 | | | NM_025065: Homo sapiens hypothetical prot | 1.02E−02 | 7.00 |
| 447574 | AF162666 | Hs. 18895 | tousled-like kinase 1 | 1.92E−04 | 6.97 |
| 447809 | AW207605 | Hs. 164230 | ESTs, Highly similar to JC7266 3′,5′-cyc | 7.31E−06 | 6.97 |
| 421379 | Y15221 | Hs. 103982 | small inducible cytokine subfamily B (Cy | 4.93E−07 | 6.97 |
| 424878 | H57111 | Hs. 221132 | ESTs | 2.67E−03 | 6.95 |
| 450016 | AA249590 | Hs. 4747 | ESTs, Weakly similar to A28996 proline-r | 8.16E−05 | 6.90 |
| 433230 | AW136134 | Hs. 220277 | ESTs | 1.45E−04 | 6.80 |
| 437967 | BE277414 | Hs. 5947 | mel transforming oncogene (derived from | 6.72E−03 | 6.80 |
| 419586 | AI088485 | Hs. 144759 | ESTs, Weakly similar to I38022 hypotheti | 1.54E−06 | 6.77 |
| 422506 | R20909 | Hs. 300741 | sorcin | 3.86E−02 | 6.77 |
| 433730 | AK002135 | Hs. 3542 | hypothetical protein FLJ11273 | 1.08E−03 | 6.72 |
| 445929 | AI089660 | Hs. 323401 | dpy-30-like protein | 4.78E−04 | 6.72 |
| 405268 | | | ENSP00000223174*: KIAA0783 PROTEIN. | 2.96E−04 | 6.70 |
| 452973 | H88409 | Hs. 40527 | ESTs | 5.20E−03 | 6.70 |
| 452744 | AI267652 | Hs. 246107 | Homo sapiens mRNA; cDNA DKFZp434E082 (fr | 5.70E−07 | 6.61 |
| 419407 | AW410377 | Hs. 41502 | hypothetical protein FLJ21276 | 4.25E−05 | 6.60 |
| 443547 | AW271273 | | hypothetical protein FLJ12666 | 5.23E−06 | 6.57 |
| 416475 | T70298 | | gb: yd26g02.s1 Soares fetal liver spleen | 2.32E−03 | 6.55 |
| 441976 | AA428403 | Hs. 106131 | ESTs | 2.50E−04 | 6.55 |
| 446493 | AK001389 | Hs. 15144 | hypothetical protein DKFZp564O043 | 4.54E−04 | 6.55 |
| 408096 | BE250162 | | dihydrofolate reductase | 1.39E−02 | 6.52 |
| 434375 | BE277910 | Hs. 3833 | 3′-phosphoadenosine 5′-phosphosulfate sy | 2.61E−05 | 6.52 |
| 453887 | BE564037 | Hs. 36237 | hypothetical protein | 1.61E−04 | 6.52 |
| 419550 | D50918 | Hs. 90998 | KIAA0128 protein; septin 2 | 1.44E−06 | 6.50 |
| 424941 | AA128376 | Hs. 153884 | ATP binding protein associated with cell | 2.25E−04 | 6.47 |
| 416133 | NM_001683 | Hs. 89512 | ATPase, Ca transporting, plasma membrane | 5.20E−03 | 6.45 |
| 442993 | BE018682 | Hs. 166196 | ATPase, Class I, type 8B, member 1 | 4.00E−05 | 6.40 |
| 445525 | BE149866 | Hs. 14831 | Homo sapiens, Similar to zinc finger pro | 1.91E−05 | 6.35 |
| 450293 | N36754 | Hs. 171118 | hypothetical protein FLJ00026 | 2.52E−04 | 6.34 |
| 409052 | AW898179 | Hs. 50123 | zinc finger protein 189 | 6.72E−03 | 6.33 |
| 442679 | R53718 | | hypothetical protein FLJ10659 | 8.87E−06 | 6.27 |
| 418476 | AA648431 | Hs. 37883 | hypothetical protein PNAS-131 | 3.67E−04 | 6.25 |
| 426925 | NM_001196 | Hs. 315689 | Homo sapiens cDNA: FLJ22373 fis, clone H | 9.71E−05 | 6.25 |
| 451788 | BE242857 | Hs. 27021 | hypothetical protein FLJ11159 | 7.26E−05 | 6.22 |
| 414183 | AW957446 | Hs. 301711 | ESTs | 1.45E−03 | 6.17 |
| 425073 | W39609 | Hs. 22003 | solute carrier family 6 (neurotransmitte | 1.05E−02 | 6.17 |
| 426110 | NM_002913 | Hs. 166563 | replication factor C (activator 1) 1 (14 | 3.33E−05 | 6.13 |
| 452548 | AL050321 | Hs. 301532 | CRP2 binding protein | 3.29E−04 | 6.08 |
| 433745 | AF075320 | Hs. 28980 | hypothetical protein FLJ14540 | 3.83E−03 | 6.07 |
| 422173 | BE385828 | Hs. 250619 | phorbolin-like protein MDS019 | 7.58E−07 | 6.05 |
| 451406 | AI694320 | Hs. 6295 | ESTs, Weakly similar to T17248 hypotheti | 1.25E−03 | 6.04 |
| 443852 | AI679966 | Hs. 150603 | ESTs | 7.94E−03 | 6.02 |
| 445813 | Z42023 | Hs. 106576 | alanine-glyoxylate aminotransferase 2-li | 6.52E−04 | 6.02 |
| 413922 | AI535895 | Hs. 221024 | ESTs | 4.57E−03 | 6.00 |
| 426458 | D83032 | Hs. 169984 | nuclear protein | 8.28E−03 | 6.00 |
| 443035 | Z45822 | Hs. 8906 | Homo sapiens clone 24889 mRNA sequence | 1.44E−04 | 5.97 |
| 447164 | AF026941 | Hs. 17518 | vipirin; similar to inflammatory respon | 8.74E−07 | 5.97 |
| 431604 | AF175265 | Hs. 264190 | vacuolar protein sorting 35 (yeast homol | 8.84E−04 | 5.95 |
| 445943 | AW898533 | Hs. 181574 | ESTs | 6.87E−04 | 5.95 |
| 451122 | AA015767 | Hs. 84522 | ESTs | 7.62E−03 | 5.95 |
| 411252 | AB018549 | Hs. 69328 | MD-2 protein | 2.60E−05 | 5.92 |
| 423598 | BE247600 | Hs. 155538 | ESTs | 9.75E−04 | 5.92 |
| 410361 | BE391804 | Hs. 62661 | guanylate binding protein 1, interferon- | 1.74E−03 | 5.88 |
| 417228 | AL134324 | Hs. 7312 | ESTs | 4.31E−04 | 5.88 |
| 434421 | AI915927 | Hs. 34771 | ESTs | 9.75E−04 | 5.87 |
| 424308 | AW975531 | Hs. 154443 | minichromosome maintenance deficient (S. | 2.17E−05 | 5.82 |
| 427484 | N32859 | Hs. 37288 | nuclear receptor subfamily 1, group D, m | 2.45E−05 | 5.82 |
| 430261 | AA305127 | Hs. 4147 | hypothetical protein HT023 | 2.42E−03 | 5.82 |
| 443291 | AA325633 | Hs. 136102 | KIAA0853 protein | 3.06E−03 | 5.82 |
| 447735 | AA775268 | Hs. 6127 | Homo sapiens cDNA: FLJ23020 fis, clone L | 5.86E−04 | 5.82 |
| 419644 | AU076951 | Hs. 179573 | retinoblastoma-binding protein 1 | 6.08E−05 | 5.80 |
| 456619 | AV647917 | Hs. 107153 | inhibitor of growth family, member 1-lik | 7.31E−06 | 5.80 |
| 414812 | X72755 | Hs. 77367 | monokine induced by gamma interferon | 1.34E−06 | 5.78 |
| 430556 | AW967807 | Hs. 13797 | ESTs | 1.83E−03 | 5.77 |
| 408360 | AI806090 | Hs. 44344 | hypothetical protein FLJ20534 | 4.24E−05 | 5.75 |
| 418224 | AL036057 | Hs. 83795 | interferon regulatory factor 2 | 2.38E−04 | 5.75 |
| 418876 | AA740616 | | gb: ny97f11.s1 NCI_CGAP_GCB1 Homo sapiens | 5.09E−05 | 5.75 |
| 456236 | AF045229 | Hs. 82280 | regulator of G-protein signalling 10 | 1.52E−03 | 5.72 |
| 437374 | AL359571 | Hs. 44054 | ninein (GSK3B interacting protein) | 6.85E−05 | 5.71 |
| 445529 | H14421 | Hs. 180513 | ATP-binding cassette, sub-family A (ABC1 | 9.77E−04 | 5.70 |
| 400517 | | | lengsin | 2.45E−05 | 5.67 |
| 410577 | X91911 | Hs. 64639 | glioma pathogenesis-related protein | 1.76E−06 | 5.67 |
| 434210 | AA665612 | | ESTs | 3.27E−06 | 5.67 |
| 438011 | BE466173 | Hs. 145696 | splicing factor (CC1.3) | 7.94E−03 | 5.67 |
| 421594 | R45689 | Hs. 21889 | Homo sapiens cDNA FLJ12978 fis, clone NT | 3.50E−03 | 5.65 |
| 400133 | | | Eos Control | 1.29E−02 | 5.63 |
| 452827 | AI571835 | Hs. 55468 | ESTs | 2.61E−05 | 5.63 |
| 448694 | AA478756 | Hs. 194477 | E3 ubiquitin ligase SMURF2 | 2.26E−04 | 5.62 |
| 408214 | AL120445 | Hs. 77823 | hypothetical protein FLJ21343 | 2.66E−04 | 5.60 |
| 449609 | BE246434 | Hs. 289026 | guanine nucleotide binding protein (G pr | 6.17E−04 | 5.59 |
| 409277 | T05558 | Hs. 156880 | ESTs | 6.17E−03 | 5.57 |
| 421650 | AA781795 | Hs. 122587 | ESTs | 1.22E−05 | 5.57 |
| 428172 | U09367 | Hs. 182828 | zinc finger protein 136 (clone pHZ-20) | 2.32E−03 | 5.57 |
| 433037 | NM_014158 | Hs. 279938 | HSPC067 protein | 3.77E−05 | 5.57 |
| 413509 | BE145419 | | gb: IL5-HT0198-291099-009-E01 HT0198 Homo | 1.75E−03 | 5.52 |
| 457130 | NM_005651 | Hs. 183671 | tryptophan 2,3-dioxygenase | 5.91E−03 | 5.52 |
| 433208 | AW002834 | Hs. 24095 | ESTs | 5.39E−02 | 5.51 |
| 410867 | X63556 | Hs. 750 | fibrillin 1 (Marfan syndrome) | 1.96E−02 | 5.50 |
| 412802 | U41518 | Hs. 74602 | aquaporin 1 (channel-forming integral pr | 5.89E−04 | 5.50 |
| 416309 | R84694 | Hs. 79194 | cAMP responsive element binding protein | 6.82E−05 | 5.50 |
| 437730 | AW071087 | Hs. 239176 | insulin-like growth factor 1 receptor | 2.54E−03 | 5.50 |
| 420520 | AK001978 | Hs. 98510 | similar to rab11-binding protein | 6.16E−03 | 5.47 |
| 422423 | AF283777 | Hs. 116481 | CD72 antigen | 5.67E−03 | 5.47 |
| 431586 | AW971100 | Hs. 293189 | ESTs | 2.79E−03 | 5.47 |
| 415660 | AI909007 | Hs. 78563 | ubiquitin-conjugating enzyme E2G 1 (homo | 1.96E−02 | 5.45 |
| 424243 | AI949359 | Hs. 143600 | ESTs, Highly similar to cis Golgi-locali | 9.29E−04 | 5.45 |
| 439008 | AF075072 | Hs. 167535 | ESTs, Weakly similar to ALU1_HUMAN ALU S | 2.66E−03 | 5.45 |
| 443441 | AW291196 | Hs. 92195 | ESTs | 4.42E−02 | 5.45 |
| 413010 | AA393273 | Hs. 75133 | transcription factor 6-like 1 (mitochond | 4.77E−03 | 5.42 |
| 439375 | AA689526 | Hs. 344249 | steroid dehydrogenase homolog | 9.70E−05 | 5.42 |
| 405141 | | | zinc finger protein 200 | 1.69E−02 | 5.40 |
| 429747 | M87507 | Hs. 2490 | caspase 1, apoptosis-related cysteine pr | 1.39E−05 | 5.40 |
| 448901 | AK001021 | Hs. 22505 | hypothetical protein FLJ10159 | 6.40E−06 | 5.40 |
| 450206 | AI796450 | Hs. 201600 | ESTs | 2.52E−04 | 5.38 |
| 437175 | AW968078 | Hs. 87773 | protein kinase, cAMP-dependent, catalyti | 2.96E−04 | 5.37 |
| 429588 | AI080271 | | ESTs | 2.55E−03 | 5.36 |
| 408048 | NM_007203 | Hs. 42322 | A kinase (PRKA) anchor protein 2 | 1.01E−02 | 5.35 |
| 422553 | AI697720 | Hs. 171455 | ESTs, Weakly similar to T31613 hypotheti | 2.31E−05 | 5.35 |
| 448554 | NM_016169 | Hs. 21431 | suppressor of fused | 2.80E−04 | 5.33 |
| 420338 | AA825595 | Hs. 88269 | Homo sapiens, clone MGC: 17339, mRNA, com | 4.92E−06 | 5.32 |
| 425167 | AA351629 | Hs. 225567 | ESTs | 2.22E−03 | 5.31 |
| 449429 | AA054224 | Hs. 59847 | ESTs | 1.10E−02 | 5.30 |
| 436562 | H71937 | Hs. 322904 | ESTs, Weakly similar to I38022 hypotheti | 4.77E−03 | 5.29 |
| 407347 | AA829847 | | gb: od40d07.s1 NCI_CGAP_GCB1 Homo sapiens | 5.08E−05 | 5.29 |
| 433505 | AW504027 | Hs. 15301 | Homo sapiens cDNA FLJ12596 fis, clone NT | 1.13E−03 | 5.27 |
| 439301 | AA833784 | Hs. 252888 | ESTs | 1.57E−02 | 5.27 |
| 439195 | H89360 | | gb: yw28d08.s1 Morton Fetal Cochlea Homo | 3.20E−03 | 5.27 |
| 414737 | AI160386 | Hs. 125087 | ESTs | 6.37E−06 | 5.25 |
| 419490 | NM_006144 | Hs. 90708 | granzyme A (granzyme 1, cytotoxic T-lymp | 1.29E−02 | 5.25 |
| 439971 | W32474 | Hs. 301746 | RAP2A, member of RAS oncogene family | 1.39E−05 | 5.25 |
| 450697 | AW152166 | Hs. 182113 | ESTs | 5.43E−03 | 5.25 |
| 432441 | AW292425 | Hs. 163484 | ESTs | 2.52E−04 | 5.22 |
| 410511 | AA743475 | Hs. 285655 | ESTs | 4.00E−03 | 5.20 |
| 421535 | AB002359 | Hs. 105478 | phosphoribosylformylglycinamidine syntha | 1.59E−03 | 5.20 |
| 427699 | AW965076 | Hs. 180378 | hypothetical protein 669 | 1.14E−02 | 5.20 |
| 437650 | AA814338 | Hs. 292297 | ESTs | 7.30E−03 | 5.20 |
| 408405 | AK001332 | Hs. 44672 | hypothetical protein FLJ10470 | 1.77E−06 | 5.18 |
| 413007 | BE046662 | | gb: hn42f02.x1 NCI_CGAP_RDF2 Homo sapiens | 8.00E−04 | 5.17 |
| 449365 | AW968261 | Hs. 118913 | ESTs, Moderately similar to T46371 hypot | 1.10E−02 | 5.17 |
| 452327 | AK000196 | Hs. 29052 | hypothetical protein FLJ20189 | 5.73E−05 | 5.17 |
| 416701 | R94977 | Hs. 35416 | PRO0132 protein | 5.20E−03 | 5.15 |
| 447774 | BE018118 | Hs. 19554 | chromosome 1 open reading frame 2 | 1.10E−02 | 5.14 |
| 437834 | AA769294 | | gb: nz36g03.s1 NCI CGAP_GCB1 Homo sapiens | 2.64E−04 | 5.13 |
| 458965 | AA010319 | Hs. 60389 | ESTs | 1.38E−03 | 5.13 |
| 421097 | AI280112 | Hs. 125232 | Homo sapiens cDNA FLJ13266 fis, clone OV | 3.26E−02 | 5.10 |
| 410337 | M83822 | Hs. 62354 | cell division cycle 4-like | 4.31E−04 | 5.10 |
| 422267 | AB033044 | Hs. 114012 | KIAA1218 protein | 4.28E−04 | 5.10 |
| 442878 | AI868648 | Hs. 22315 | ESTs | 1.92E−03 | 5.10 |
| 451338 | AW612322 | Hs. 19131 | transcription factor Dp-2 (E2F dimerizat | 2.66E−04 | 5.10 |
| 407204 | R41933 | Hs. 140237 | ESTs, Weakly similar to ALU1_HUMAN ALU S | 2.52E−04 | 5.07 |
| 431049 | AA846576 | Hs. 103267 | hypothetical protein FLJ22548 similar to | 8.59E−05 | 5.07 |
| 419522 | AI682428 | Hs. 157728 | ESTs | 2.13E−04 | 5.05 |
| 431736 | AI912234 | Hs. 3297 | ribosomal protein S27a | 9.76E−04 | 5.02 |
| 433697 | AA600357 | Hs. 239489 | TIA1 cytotoxic granute-associated RNA-bi | 1.34E−02 | 5.02 |
| 403738 | | | C4000675*: gi\|3426332\|gb\|AAC32272.1\| (AF0 | 7.30E−06 | 5.02 |
| 453742 | AB037744 | Hs. 34892 | KIAA1323 protein | 3.35E−03 | 5.02 |
| 426052 | N49068 | Hs. 93966 | ESTs | 2.96E−04 | 5.00 |
| 430268 | AK000737 | Hs. 237480 | hypothetical protein FLJ20730 | 4.56E−03 | 5.00 |
| 430512 | AF182294 | Hs. 241578 | U6 snRNA-associated Sm-like protein LSm8 | 1.39E−02 | 5.00 |
| 434658 | AI624436 | Hs. 310286 | ESTs | 1.22E−04 | 5.00 |
| 450086 | AW016343 | Hs. 233301 | ESTs | 4.98E−03 | 5.00 |
| 432606 | NM_002104 | Hs. 3066 | granzyme K (serine protease, granzyme 3; | 8.15E−05 | 4.99 |
| 450916 | AA011597 | Hs. 177398 | ESTs | 4.98E−03 | 4.98 |
| 444372 | AW377983 | Hs. 298140 | Homo sapiens cDNA: FLJ22502 fis, clone H | 8.13E−05 | 4.98 |
| 439334 | AI148976 | Hs. 112062 | ESTs | 9.71E−04 | 4.98 |
| 440043 | BE277457 | Hs. 30661 | hypothetical protein MGC4606 | 1.52E−03 | 4.97 |
| 424852 | AI222779 | Hs. 144848 | ESTs | 1.06E−02 | 4.95 |
| 443884 | N20617 | Hs. 194397 | leptin receptor | 2.54E−02 | 4.95 |
| 444670 | H58373 | Hs332938 | hypothetical protein MGC5370 | 2.96E−04 | 4.95 |
| 409703 | NM_006187 | Hs. 56009 | 2′-5′-oligoadenylate synthetase 3 (100 k | 1.74E−07 | 4.94 |
| 426793 | X89887 | Hs. 172350 | HIR (histone cell cycle regulation defec | 4.18E−03 | 4.93 |
| 407366 | AF026942 | Hs. 17518 | gb: Homo sapiens cig33 mRNA, partial sequ | 3.76E−06 | 4.92 |
| 428255 | AI627478 | Hs. 187670 | ESTs | 3.51E−03 | 4.91 |
| 415938 | BE383507 | Hs. 78921 | A kinase (PRKA) anchor protein 1 | 6.44E−05 | 4.91 |
| 403904 | | | ENSP00000251503*: WUGSC | 7.31E−03 | 4.90 |
| 408831 | AF090114 | Hs. 48433 | endocrine regulator | 1.81E−04 | 4.90 |
| 414646 | AA353776 | Hs. 901 | CD48 antigen (B-cell membrane protein) | 7.82E−06 | 4.90 |
| 420151 | AA255931 | Hs. 186704 | ESTs | 8.60E−05 | 4.90 |
| 434158 | T86534 | Hs. 14372 | ESTs | 2.43E−03 | 4.90 |
| 450447 | AF212223 | Hs. 25010 | hypothetical protein P15-2 | 3.99E−03 | 4.90 |
| 447769 | AW873704 | Hs. 320831 | Homo sapiens cDNA FLJ14597 fis, clone NT | 4.79E−04 | 4.89 |
| 416050 | U51903 | Hs. 78993 | IQ motif containing GTPase activating pr | 3.14E−02 | 4.88 |
| 421215 | AI868634 | Hs. 246358 | ESTs, Weakly similar to T32250 hypotheti | 3.99E−05 | 4.87 |
| 408761 | AA057264 | Hs. 238936 | ESTs, Weakly similar to (defline not ava | 3.48E−04 | 4.86 |
| 449523 | NM_000579 | Hs. 54443 | chemokine (C-C motif) receptor 5 | 2.78E−05 | 4.85 |
| 423857 | N48902 | Hs. 133481 | Homo sapiens mRNA; cDNA DKFZp564O0862 (f | 1.66E−03 | 4.82 |
| 427384 | T82854 | | gb: yd42a09.r1 Soares fetal liver spleen | 1.14E−02 | 4.82 |
| 451273 | NM_014811 | Hs. 26163 | KIAA0649 gene product | 4.35E−03 | 4.82 |
| 431620 | AA126109 | Hs. 264981 | 2′-5′-oligoadenylate synthetase 2 (69-71 | 1.75E−07 | 4.82 |
| 439223 | AW238299 | Hs. 250618 | UL16 binding protein 2 | 5.03E−04 | 4.82 |
| 451081 | AI078645 | Hs. 431 | murine leukemia viral (bmi-1) oncogene h | 7.30E−03 | 4.79 |
| 407609 | R43159 | Hs. 238432 | ESTs | 1.92E−03 | 4.77 |
| 424683 | N87519 | Hs. 27196 | ESTs | 2.52E−04 | 4.77 |
| 429966 | BE081342 | Hs. 283037 | HSPC039 protein | 6.93E−02 | 4.77 |
| 443998 | AI620661 | Hs. 296276 | ESTs | 8.63E−03 | 4.77 |
| 452820 | N46161 | Hs. 35274 | ESTs | 5.43E−03 | 4.77 |
| 454075 | R43826 | Hs. 16313 | Kruppel-like zinc finger protein GLIS2 | 7.30E−03 | 4.77 |
| 429922 | Z97630 | Hs. 226117 | H1 histone family, member 0 | 3.25E−02 | 4.77 |
| 422541 | NM_005131 | Hs. 1540 | nuclear matrix protein p84 | 2.66E−03 | 4.76 |
| 449613 | N63808 | Hs. 34299 | ESTs | 5.99E−06 | 4.75 |
| 407309 | AA526438 | Hs. 281680 | peroxisomal trans 2-enoyl CoA reductase; | 7.68E−05 | 4.74 |
| 431966 | AB037903 | Hs. 272257 | Homo sapiens truncated AKR mRNA for trun | 7.62E−03 | 4.73 |
| 410382 | AW664971 | Hs. 259546 | ESTs | 7.68E−05 | 4.72 |
| 434926 | BE543269 | Hs. 50252 | mitochondrial ribosomal protein L32 | 3.29E−04 | 4.72 |
| 414219 | W20010 | Hs. 75823 | ALL1-fused gene from chromosome 1q | 8.62E−03 | 4.71 |
| 413048 | M93221 | Hs. 75182 | mannose receptor, C type 1 | 1.91E−04 | 4.67 |
| 419110 | AA234171 | Hs. 187626 | ESTs | 1.71E−04 | 4.67 |
| 429952 | AF080158 | Hs. 226573 | inhibitor of kappa light polypeptide gen | 2.52E−04 | 4.67 |
| 450401 | AW959281 | Hs. 8184 | ESTs | 1.19E−03 | 4.67 |
| 432600 | AI821085 | | gb: ns95a12.y5 NCI_CGAP_Pr3 Homo sapiens | 2.55E−03 | 4.65 |
| 441892 | AB028981 | Hs. 8021 | KIAA1058 protein | 1.29E−04 | 4.65 |
| 446207 | AW968535 | Hs. 14328 | hypothetical protein FLJ20071 | 9.27E−04 | 4.65 |
| 450516 | AA902656 | Hs. 21943 | NIF3 (Ngg1 interacting factor 3. S. pombe | 7.19E−04 | 4.65 |
| 419590 | AF005043 | Hs. 91390 | poly (ADP-ribose) glycohydrolase | 6.44E−05 | 4.65 |
| 449909 | AA004681 | Hs. 59432 | ESTs | 4.77E−03 | 4.63 |
| 409401 | AI201895 | Hs. 181309 | proteasome (prosome, macropain) subunit. | 2.12E−03 | 4.63 |
| 441652 | BE467811 | Hs. 7471 | BBP-like protein 1 | 1.75E−03 | 4.61 |
| 417688 | R09170 | Hs. 284350 | ESTs | 4.78E−04 | 4.61 |
| 411605 | AW006831 | | ESTs | 8.26E−03 | 4.60 |
| 408108 | AI580492 | Hs. 42743 | hypothetical protein | 1.38E−03 | 4.60 |
| 451079 | AI827988 | Hs. 240728 | ESTs, Moderately similar to PC4259 ferri | 8.00E−04 | 4.60 |
| 451326 | AW296946 | Hs. 256078 | ESTs | 5.57E−04 | 4.60 |
| 453555 | N23574 | Hs. 123649 | ESTs, Moderately similar to ALU7_HUMAN A | 6.81E−05 | 4.60 |
| 447541 | AK000288 | Hs. 18800 | hypothetical protein FLJ20281 | 2.19E−02 | 4.60 |
| 402737 | | | Target Exon | 3.66E−04 | 4.60 |
| 454067 | AA041455 | | ESTs | 8.00E−04 | 4.59 |
| 408705 | AA312135 | Hs. 46967 | HSPCO34 protein | 3.06E−06 | 4.57 |
| 413786 | AW613780 | Hs. 13500 | ESTs | 5.69E−07 | 4.57 |
| 424626 | AA344308 | Hs. 128427 | Homo sapiens BAC clone RP11-335J18 from | 2.45E−05 | 4.57 |
| 447023 | AA356764 | Hs. 17109 | integral membrane protein 2A | 1.92E−04 | 4.57 |
| 449500 | AW956345 | Hs. 12926 | ESTs | 5.56E−02 | 4.56 |
| 432572 | AI660840 | Hs. 191202 | ESTs, Weakly similar to ALUE_HUMAN !!!! | 1.06E−02 | 4.55 |
| 446927 | AW503484 | Hs. 16533 | myosin phosphatase, target subunit 1 | 2.78E−03 | 4.55 |
| 452695 | AW780199 | Hs. 30327 | mitogen-activated protein kinase-activat | 2.32E−03 | 4.55 |
| 456034 | AW450979 | | gb: UI-H-BI3-ala-a-12-0-UI.s1 NCI_CGAP_Su | 3.26E−02 | 4.55 |
| 401091 | | | decay accelerating factor for complement | 7.98E−04 | 4.52 |
| 423250 | BE061916 | Hs 125849 | chromosome 8 open reading frame 2 | 1.89E−02 | 4.52 |
| 451690 | AW451469 | Hs. 209990 | ESTs | 1.06E−02 | 4.52 |
| 443119 | AA312264 | Hs. 7980 | hypothetical protein MGC12966 | 1.91E−04 | 4.50 |
| 410245 | C17908 | Hs. 194125 | ESTs | 1.69E−02 | 4.49 |
| 448760 | AA313825 | Hs. 323583 | AD036 protein | 8.13E−05 | 4.48 |
| 417954 | AI633943 | Hs. 26613 | ESTs, Weakly similar to no similarities | 5.56E−02 | 4.47 |
| 417973 | NM_004490 | Hs. 83070 | growth factor receptor-bound protein 14 | 2.73E−02 | 4.47 |
| 419743 | AW408762 | Hs. 5957 | Homo sapiens clone 24416 mRNA sequence | 4.88E−02 | 4.47 |
| 429276 | AF056085 | Hs. 198612 | G protein-coupled receptor 51 | 2.80E−04 | 4.47 |
| 432967 | AA572949 | Hs. 207566 | ESTs | 7.59E−03 | 4.47 |
| 433226 | AW503733 | Hs. 9414 | KIAA1488 protein | 2.67E−03 | 4.47 |
| 445776 | NM_001310 | Hs. 13313 | cAMP responsive element binding protein- | 2.83E−02 | 4.47 |
| 426860 | U04953 | Hs. 172801 | isoleucine-tRNA synthetase | 1.34E−02 | 4.47 |
| 419943 | AA252111 | Hs. 15200 | ESTs | 4.54E−04 | 4.47 |
| 442202 | BE272862 | Hs. 106534 | hypothetical protein FLJ22625 | 4.30E−04 | 4.47 |
| 432676 | AI187366 | | gb: qf29c01.x1 Soares_testis_NHT Homo sap | 1.45E−04 | 4.46 |
| 421181 | NM_005574 | Hs. 184585 | LIM domain only 2 (rhombotin-like 1) | 5.91E−03 | 4.45 |
| 428342 | AI739168 | | Homo sapiens cDNA FLJ13458 fis, clone PL | 8.15E−05 | 4.45 |
| 434941 | AW073202 | Hs. 334825 | Homo sapiens cDNA FLJ14752 fis, clone NT | 7.95E−03 | 4.45 |
| 446839 | BE091926 | Hs. 16244 | mitotic spindle coiled-coil related prot | 4.56E−03 | 4.45 |
| 421508 | NM_004833 | Hs. 105115 | absent in melanoma 2 | 1.53E−04 | 4.43 |
| 438098 | AI076370 | Hs. 134037 | ESTs | 1.19E−02 | 4.42 |
| 449001 | AI619957 | | ESTs | 6.17E−03 | 4.42 |
| 409461 | AA382169 | Hs. 54483 | N-myc (and STAT) interactor | 4.91E−07 | 4.42 |
| 408461 | AB037756 | Hs. 45207 | hypothetical protein KIAA1335 | 8.26E−03 | 4.40 |
| 413243 | AA769266 | Hs. 193657 | ESTs | 1.06E−02 | 4.40 |
| 450747 | AI064821 | Hs. 318535 | ESTs, Highly similar to 1818357A EWS gen | 5.92E−02 | 4.40 |
| 451658 | AW195351 | Hs. 250520 | ESTs, Moderately similar to I38022 hypot | 1.22E−04 | 4.38 |
| 419951 | AI653415 | Hs. 195789 | ESTs | 8.63E−05 | 4.38 |
| 421685 | AF189723 | Hs. 106778 | ATPase, Ca transporting, type 2C, member | 8.00E−04 | 4.38 |
| 424960 | BE245380 | Hs. 153952 | 5′ nucleotidase (CD73) | 1.06E−02 | 4.38 |
| 432435 | BE218886 | Hs. 282070 | ESTs | 1.19E−03 | 4.38 |
| 431863 | AA188185 | Hs. 289043 | spindlin | 3.55E−05 | 4.37 |
| 408162 | AA993833 | Hs. 118527 | ESTs | 6.41E−03 | 4.35 |
| 417363 | AW129357 | Hs. 329700 | ESTs | 9.37E−03 | 4.35 |
| 422879 | AI241409 | Hs. 188092 | ESTs | 1.59E−03 | 4.35 |
| 438769 | AA830684 | Hs. 163426 | ESTs | 2.79E−03 | 4.35 |
| 443084 | AI827639 | Hs. 125539 | ESTs | 2.66E−03 | 4.35 |
| 425068 | AL048716 | Hs. 154387 | KIAA0103 gene product | 8.08E−02 | 4.35 |
| 445652 | AL117473 | Hs. 13036 | DKFZP727A071 protein | 8.40E−04 | 4.33 |
| 431328 | AA502999 | Hs. 291591 | ESTs | 2.03E−04 | 4.30 |
| 434666 | AF151103 | Hs. 112259 | T cell receptor gamma locus | 7.99E−04 | 4.30 |
| 452939 | R35348 | Hs. 24970 | ESTs, Weakly similar to B34323 GTP-bindi | 6.17E−03 | 4.30 |
| 407690 | R47799 | Hs. 266957 | hypothetical protein FLJ14281 | 3.28E−06 | 4.27 |
| 432610 | BE246615 | Hs. 278507 | histidyl-tRNA synthetase-like | 3.35E−03 | 4.27 |
| 434198 | AF119849 | Hs. 283028 | hypothetical protein PRO1598 | 2.38E−04 | 4.27 |
| 446751 | AA766998 | Hs. 79126 | Human DNA sequence from clone RP11-16L21 | 4.58E−02 | 4.27 |
| 447887 | AA114050 | Hs. 19949 | caspase 8, apoptosis-related cysteine pr | 7.96E−04 | 4.27 |
| 411656 | AW855576 | | gb: CM4-CT0278-221099-027-d01 CT0278 Homo | 3.20E−03 | 4.25 |
| 428720 | T90468 | Hs. 178154 | ESTs | 1.25E−03 | 4.25 |
| 432195 | AJ243669 | Hs. 8127 | KIAA0144 gene product | 6.52E−04 | 4.25 |
| 433847 | AA610266 | Hs. 3631 | ESTs | 4.54E−04 | 4.25 |
| 434987 | AW975114 | | ESTs | 7.62E−03 | 4.25 |
| 443020 | AI350058 | Hs. 106129 | ESTs | 1.39E−02 | 4.24 |
| 440193 | AW902312 | Hs. 7037 | Homo sapiens clone 24923 mRNA sequence | 8.57E−02 | 4.22 |
| 440201 | AL359588 | Hs. 7041 | hypothetical protein DKFZp762B226 | 1.05E−02 | 4.22 |
| 447513 | AW955776 | Hs. 313500 | ESTs, Moderately similar to ALU7_HUMAN A | 2.03E−06 | 4.22 |
| 453793 | AK002178 | Hs. 35225 | hypothetical protein FLJ11316 | 6.72E−03 | 4.22 |
| 434280 | BE005398 | | gb: CM1-BN0116-150400-189-h02 BN0116 Homo | 1.59E−03 | 4.22 |
| 414405 | AI362533 | | KIAA0306 protein | 6.39E−06 | 4.21 |
| 433437 | U20536 | Hs. 3280 | caspase 6, apoptosis-related cysteine pr | 7.81E−06 | 4.20 |
| 402507 | | | Target Exon | 5.30E−04 | 4.20 |
| 430569 | AF241254 | Hs. 178098 | angiotensin \| converting enzyme (peptidy | 2.45E−02 | 4.20 |
| 442061 | AA774284 | Hs. 285728 | abl-interactor 12 (SH3-containing protei | 7.58E−04 | 4.20 |
| 414915 | NM_002462 | Hs. 76391 | myxovirus (influenza) resistance 1, homo | 2.36E−07 | 4.19 |
| 442045 | C05768 | Hs. 8078 | Homo sapiens clone FBD3 Cri-du-chat crit | 1.31E−03 | 4.18 |
| 402964 | | | NM_022095*: Homo sapiens hypothetical C2H | 5.59E−04 | 4.17 |
| 452032 | BE244005 | Hs. 27610 | retinoic acid-and interferon-inducible | 4.54E−04 | 4.17 |
| 407218 | AA095473 | | ubiquitin-conjugating enzyme E2H (homolo | 7.93E−03 | 4.17 |
| 425387 | AB037864 | Hs. 156051 | KIAA1443 protein | 3.65E−04 | 4.17 |
| 431122 | AI267593 | Hs. 250535 | Homo sapiens mRNA; cDNA DKFZp434N2412 (f | 6.51E−04 | 4.17 |
| 407756 | AA116021 | Hs. 38260 | ubiquitin specific protease 18 | 3.41E−07 | 4.17 |
| 407992 | AW418811 | | gb: ha21a06.x1 NCI-HD — | 3.64E−03 | 4.16 |
| | | | CGAP_Kid12 Homo sapien | | |
| 416647 | BE297139 | Hs. 79411 | replication protein A2 (32 kD) | 7.98E−04 | 4.16 |
| 433017 | Y15067 | Hs. 279914 | zinc finger protein 232 | 3.31E−05 | 4.15 |
| 435513 | AW404075 | Hs. 42785 | DC11 protein | 2.63E−02 | 4.15 |
| 447094 | X65232 | Hs. 17364 | zinc finger protein 79 (pT7) | 1.19E−03 | 4.15 |
| 450746 | D82673 | Hs. 278589 | general transcription factor II, i | 5.23E−06 | 4.15 |
| 432388 | X15218 | Hs. 2969 | v-ski avian sarcoma viral oncogene homol | 8.84E−04 | 4.15 |
| 431976 | AA719001 | Hs. 291065 | ESTs | 6.86E−05 | 4.14 |
| 453394 | AW960474 | Hs. 40289 | ESTs | 103E−04 | 4.13 |
| 418793 | AW382987 | Hs. 88474 | prostaglandin-endoperoxide synthase 1 (p | 1.39E−02 | 4.13 |
| 456439 | AA251242 | Hs. 103238 | ESTs | 7.94E−03 | 4.13 |
| 406423 | | | C19000229*: gi\|6753826\|ref\|NP_034311.1\| f | 2.04E−02 | 4.13 |
| 408392 | U28831 | Hs. 44566 | KIAA1641 protein | 5.67E−03 | 4.13 |
| 417601 | NM_014735 | Hs. 82292 | KIAA0215 gene product | 3.13E−04 | 4.13 |
| 425462 | AI491852 | Hs. 46783 | Homo sapiens cDNA: FLJ22382 fis, clone H | 3.74E−02 | 4.12 |
| 440624 | AF017987 | Hs. 7306 | secreted frizzled-related protein 1 | 1.10E−02 | 4.11 |
| 422040 | AA172106 | Hs. 110950 | Rag C protein | 6.53E−04 | 4.10 |
| 416987 | D86957 | Hs. 80712 | KIAA0202 protein | 4.37E−03 | 4.10 |
| 418720 | AI381687 | Hs. 39526 | ESTs | 5.39E−02 | 4.10 |
| 433556 | W56321 | Hs. 111460 | calcium/calmodulin-dependent protein kin | 1.15E−04 | 4.10 |
| 435260 | H64245 | Hs. 34458 | ESTs | 3.35E−03 | 4.09 |
| 443601 | AI078554 | Hs. 42658 | ESTs | 1.45E−04 | 4.09 |
| 435029 | AF167706 | Hs. 19280 | cysteine-rich motor neuron 1 | 1.45E−02 | 4.08 |
| 446667 | BE161878 | Hs224805 | ESTs | 2.04E−02 | 4.06 |
| 406038 | | | zinc finger protein 200 | 2.92E−03 | 4.05 |
| 430468 | NM_004673 | Hs. 241519 | angiopoietin-like 1 | 1.69E−02 | 4.05 |
| 430522 | N75750 | Hs. 242271 | KIAA0471 gene product | 4.00E−03 | 4.05 |
| 423732 | AF058056 | Hs. 132183 | solute carrier family 16 (monocarboxylic | 2.02E−03 | 4.05 |
| 408548 | AA055449 | Hs. 63187 | ESTs, Weakly similar to ALUC_HUMAN !!!! | 3.66E−03 | 4.02 |
| 451593 | AF151879 | Hs. 26706 | CGI-121 protein | 1.71E−04 | 4.02 |
| 459297 | BE300741 | Hs. 125034 | hypothetical protein FLJ13340 | 2.92E−03 | 4.02 |
| 433001 | AF217513 | Hs. 279905 | clone HQ0310 PRO0310p1 | 3.13E−04 | 4.02 |
| 433233 | AB040927 | Hs. 301804 | KIAA1494 protein | 8.27E−03 | 4.00 |
| 445733 | BE295568 | Hs. 13225 | UDP-Gal: betaGlcNAc beta 1,4-galactosylt | 3.06E−03 | 4.00 |
| 444985 | AI677737 | Hs. 28329 | hypothetical protein FLJ14005 | 8.82E−04 | 3.98 |
| 408380 | AF123050 | Hs. 44532 | diubiquitin | 1.54E−06 | 3.98 |
| 416999 | AW195747 | Hs. 21122 | hypothetical protein FLJ11830 similar to | 5.89E−04 | 3.97 |
| 424238 | AA337401 | Hs. 137635 | ESTs | 2.93E−02 | 3.97 |
| 425395 | NM_014102 | Hs. 156243 | PRO1848 protein | 9.70E−05 | 3.97 |
| 442485 | BE092285 | Hs. 29724 | hypothetical protein FLJ13187 | 1.02E−02 | 3.97 |
| 401649 | | | Target Exon | 9.28E−04 | 3.95 |
| 426108 | AA622037 | Hs. 166468 | programmed cell death 5 | 9.92E−02 | 3.95 |
| 441889 | AI090455 | Hs. 268371 | hypothetical protein FLJ20274 | 3.74E−02 | 3.95 |
| 428467 | AK002121 | Hs. 184465 | hypothetical protein FLJ11259 | 1.13E−03 | 3.95 |
| 452194 | AI694413 | | Ubiquitin-like protein FAT10??? - diubiq | 2.18E−06 | 3.93 |
| 421443 | BE550141 | Hs. 156148 | hypothetical protein FLJ13231 | 7.59E−04 | 3.92 |
| 427213 | AW007211 | | hypothetical protein FLJ12876 | 5.21E−03 | 3.92 |
| 449964 | AW001741 | Hs. 24243 | hypothetical protein FLJ10706 | 2.01E−03 | 3.92 |
| 435970 | H75410 | Hs. 54452 | zinc finger protein, subfamily 1 A, 1 (lk | 1.44E−03 | 3.92 |
| 448965 | AF092134 | Hs. 22679 | CGI-24 protein | 5.74E−05 | 3.90 |
| 419737 | H24185 | Hs. 92918 | hypothetical protein | 1.75E−03 | 3.90 |
| 425210 | AA054679 | Hs. 155150 | ribonuclease P (14 kD) | 7.30E−03 | 3.90 |
| 437862 | AW978107 | Hs. 5884 | Homo sapiens mRNA; cDNA DKFZp586C0224 (f | 5.67E−03 | 3.90 |
| 400475 | | | NM_031436*: Homo sapiens hypothetical pro | 2.91E−03 | 3.88 |
| 433075 | NM_002959 | | sortilin 1 | 5.75E−02 | 3.88 |
| 428420 | AL096858 | Hs. 184245 | KIAA0929 protein Msx2 interacting nuclea | 5.26E−06 | 3.86 |
| 436139 | AA765786 | Hs. 120936 | ESTs | 8.16E−05 | 3.86 |
| 449509 | AA001615 | Hs. 84561 | ESTs | 4.13E−02 | 3.85 |
| 452642 | AW474296 | Hs. 29667 | ESTs | 1.24E−02 | 3.85 |
| 456107 | AA160000 | Hs. 137396 | ESTs, Weakly similar to JC5238 galactosy | 6.15E−03 | 3.85 |
| 457584 | AA147979 | Hs. 285005 | mitochondrial import receptor Tom22 | 3.13E−04 | 3.84 |
| 414658 | X58528 | Hs. 76781 | ATP-binding cassette, sub-family D (ALD) | 4.18E−03 | 3.84 |
| 452866 | R26969 | Hs. 268016 | Homo sapiens cDNA: FLJ21243 fis, clone C | 1.38E−03 | 3.84 |
| 412651 | AA115333 | Hs. 107968 | ESTs | 4.02E−06 | 3.84 |
| 403575 | | | Target Exon | 1.44E−03 | 3.82 |
| 418304 | AA215702 | | gb: zr97g10.r1 NCI_CGAP_GCB1 Homo sapiens | 1.59E−03 | 3.82 |
| 435354 | AA678267 | Hs. 117115 | ESTs | 4.42E−02 | 3.82 |
| 411400 | AA311919 | Hs. 69851 | nucleolar protein family A, member 1 (H/ | 3.12E−04 | 3.82 |
| 408618 | AK000637 | Hs. 46624 | HSPC043 protein | 1.63E−02 | 3.81 |
| 419135 | R61448 | Hs. 106728 | ESTs, Weakly similar to KIAA1353 protein | 2.16E−05 | 3.80 |
| 421965 | AA301100 | Hs. 346482 | gb: EST14128 Testis tumor Homo sapiens cD | 3.33E−05 | 3.79 |
| 419908 | AW971327 | Hs. 293315 | ESTs | 1.13E−03 | 3.79 |
| 438021 | AV653790 | | WW domain-containing protein 1 | 9.79E−04 | 3.78 |
| 423450 | AJ290445 | Hs. 128759 | KIAA0524 protein | 2.81E−04 | 3.77 |
| 432689 | AB018320 | | Arg/Abl-interacting protein ArgBP2 | 1.51E−02 | 3.77 |
| 434526 | AW085147 | Hs. 152779 | ESTs | 8.24E−03 | 3.77 |
| 436385 | BE551618 | Hs. 144097 | ESTs | 5.39E−02 | 3.77 |
| 424003 | BE274717 | Hs. 137506 | Homo sapiens, clone IMAGE: 3605104, mRNA, | 1.69E−02 | 3.75 |
| 432409 | AA806538 | Hs. 130732 | KIAA1575 protein | 5.93E−02 | 3.75 |
| 432873 | AW837268 | Hs. 279639 | Homo sapiens mRNA; cDNA DKFZp586M2022 (f | 1.14E−02 | 3.74 |
| 401016 | | | ENSP00000227126: NAALADASE II PROTEIN. | 2.92E−03 | 3.72 |
| 425397 | J04088 | Hs. 156346 | topoisomerase (DNA) II alpha (170 kD) | 3.37E−02 | 3.72 |
| 426310 | NM_000909 | Hs. 169266 | neuropeptide Y receptor Y1 | 6.17E−03 | 3.72 |
| 430016 | NM_004736 | Hs. 227656 | xenotropic and polytropic retrovirus rec | 2.93E−02 | 3.72 |
| 434733 | AI334367 | Hs. 159337 | ESTs | 4.78E−04 | 3.72 |
| 447892 | AI435848 | Hs. 172978 | ESTs | 2.63E−02 | 3.72 |
| 453753 | BE252983 | Hs. 35086 | ubiquitin specific protease 1 | 4.56E−02 | 3.72 |
| 410099 | AA081630 | | KIAA0036 gene product | 7.84E−02 | 3.72 |
| 421205 | AL137540 | Hs. 102541 | netrin 4 | 6.12E−02 | 3.71 |
| 437629 | AW574774 | Hs. 121692 | ESTs | 7.98E−04 | 3.71 |
| 446506 | AI123118 | Hs. 15159 | chemokine-like factor, alternatively spl | 1.16E−06 | 3.71 |
| 405689 | | | NM_018850*: Homo sapiens ATP-binding cass | 8.40E−04 | 3.71 |
| 427399 | NM_014883 | Hs. 177664 | KIAA0914 gene product | 4.54E−04 | 3.70 |
| 417848 | AA206581 | Hs. 116586 | ESTs, Weakly similar to JC5314 CDC28/cdc | 1.75E−03 | 3.70 |
| 419511 | AA429750 | Hs. 75113 | general transcription factor IIIA | 5.20E−03 | 3.70 |
| 425423 | NM_005897 | Hs. 157180 | intracisternal A particle-promoted polyp | 1.44E−03 | 3.70 |
| 433891 | AA613792 | | gb: no97h03.s1 NCI_CGAP_Pr2 Homo sapiens | 2.19E−02 | 3.70 |
| 434924 | AA443164 | Hs. 23259 | hypothetical protein FLJ13433 | 4.01E−05 | 3.70 |
| 442085 | AA975688 | Hs. 159955 | ESTs | 4.00E−03 | 3.70 |
| 444363 | AI142827 | Hs. 143656 | ESTs | 4.29E−04 | 3.70 |
| 423954 | AW753164 | Hs. 288604 | KIAA1632 protein | 2.11E−03 | 3.69 |
| 423494 | AW504365 | Hs. 24143 | Wiskott-Aldrich syndrome protein interac | 2.54E−03 | 3.67 |
| 429680 | AL035754 | Hs. 2474 | toll-like receptor 1 | 1.62E−04 | 3.67 |
| 429686 | AI871613 | Hs. 28538 | Homo sapiens cDNA: FLJ21086 fis, clone C | 1.75E−02 | 3.67 |
| 440865 | AI281525 | Hs. 130180 | ESTs | 2.43E−03 | 3.67 |
| 444508 | AI554691 | Hs. 334583 | ring finger protein 23 | 1.02E−03 | 3.67 |
| 427581 | NM_014788 | Hs. 179703 | KIAA0129 gene product | 4.92E−07 | 3.67 |
| 438459 | T49300 | Hs. 35304 | Homo sapiens cDNA FLJ13655 fis, clone PL | 2.38E−04 | 3.67 |
| 442160 | AI337127 | Hs. 156325 | ESTs | 4.00E−06 | 3.67 |
| 448071 | BE621584 | Hs. 6983 | Homo sapiens cDNA: FLJ22646 fis, clone H | 1.62E−04 | 3.65 |
| 425757 | AA363171 | | gb: EST72986 Ovary II Homo sapiens cDNA 5 | 1.13E−03 | 3.65 |
| 431560 | BE244135 | Hs. 260238 | hypothetical protein FLJ10842 | 5.19E−03 | 3.65 |
| 444969 | AI203334 | Hs. 160628 | ESTs | 7.93E−03 | 3.65 |
| 443562 | AF118838 | Hs. 9599 | solute carrier family 25, member 13 (cit | 2.04E−02 | 3.64 |
| 435511 | AA683336 | Hs. 189046 | ESTs | 1.89E−02 | 3.64 |
| 436503 | AJ277750 | Hs. 183924 | ubiquitin associated and SH3 domain cont | 5.67E−03 | 3.63 |
| 417148 | AA359896 | Hs. 293885 | hypothetical protein FLJ14902 | 6.31E−02 | 3.63 |
| 421114 | AW975051 | Hs. 293156 | ESTs, Weakly similar to 178885 serine/th | 2.73E−02 | 3.63 |
| 443373 | AI792868 | Hs. 135365 | ESTs | 1.06E−02 | 3.63 |
| 449720 | AA311152 | Hs. 288708 | hypothetical protein FLJ21562 | 3.99E−05 | 3.63 |
| 451938 | AI354355 | Hs. 16697 | down-regulator of transcription 1, TBP-b | 1.38E−03 | 3.63 |
| 457231 | AI472022 | Hs. 301959 | proline synthetase co-transcribed (bacte | 1.02E−02 | 3.63 |
| 425332 | AA633306 | Hs. 127279 | ESTs | 9.28E−04 | 3.61 |
| 436860 | H12751 | Hs. 5327 | PRO1914 protein | 4.08E−04 | 3.60 |
| 400281 | | | Eos Control | 2.96E−05 | 3.60 |
| 407946 | AA226495 | Hs. 154292 | ESTs | 5.83E−04 | 3.60 |
| 418699 | BE539639 | Hs. 173030 | ESTs, Weakly similar to ALU8_HUMAN ALU S | 1.10E−02 | 3.60 |
| 422303 | AW410382 | Hs. 27556 | hypothetical protein FLJ22405 | 6.32E−02 | 3.60 |
| 425345 | AU077297 | Hs. 155894 | protein tyrosine phosphatase, non-recept | 6.44E−03 | 3.60 |
| 425692 | D90041 | Hs. 155956 | N-acetyltransferase 1 (arylamine N-acety | 7.67E−05 | 3.60 |
| 428250 | AW809208 | Hs. 183297 | DKFZP566F2124 protein | 1.13E−03 | 3.60 |
| 443968 | AA287702 | Hs. 10031 | KIAA0955 protein | 1.51E−02 | 3.60 |
| 447433 | AA651869 | Hs. 5320 | hypothetical protein | 1.44E−02 | 3.60 |
| 456760 | AW961251 | Hs. 127828 | guanine nucleotide binding protein (G pr | 1.31E−03 | 3.60 |
| 430293 | AI416988 | Hs. 238272 | inositol 1,4,5-triphosphate receptor, ty | 2.14E−04 | 3.59 |
| 427051 | BE178110 | Hs. 173374 | Homo sapiens cDNA FLJ10500 fis, clone NT | 3.49E−02 | 3.58 |
| 408438 | AB011180 | Hs. 100960 | KIAA0608 protein | 6.17E−03 | 3.57 |
| 413645 | AA130992 | | gb: zo15e02.s1 Stratagene colon (937204) | 1.63E−02 | 3.57 |
| 424881 | AL119690 | Hs. 153618 | HCGVIII-1 protein | 6.17E−03 | 3.57 |
| 427471 | AA403131 | Hs. 266782 | KIAA1826 protein | 4.37E−03 | 3.57 |
| 430024 | AI808780 | Hs. 227730 | integrin, alpha 6 | 2.02E−04 | 3.57 |
| 447941 | AW181928 | Hs. 249946 | ESTs | 9.64E−02 | 3.57 |
| 418822 | Z43371 | Hs. 7012 | ESTs | 1.19E−03 | 3.56 |
| 409342 | AU077058 | Hs. 54089 | BRCA1 associated RING domain 1 | 6.72E−03 | 3.55 |
| 410054 | AL120050 | Hs. 58220 | Homo sapiens cDNA: FLJ23005 fis, clone L | 8.27E−03 | 3.55 |
| 419440 | AB020689 | Hs. 90419 | KIAA0882 protein | 7.61E−04 | 3.55 |
| 423886 | AA332098 | | gb: EST36256 Embryo, 8 week I Homo sapien | 2.21E−03 | 3.55 |
| 437133 | AB018319 | Hs. 5460 | KIAA0776 protein | 4.14E−02 | 3.55 |
| 445718 | H79791 | Hs. 15227 | ESTs | 2.78E−05 | 3.55 |
| 408731 | R85652 | | Homo sapiens mRNA; cDNA DKFZp434F1928 (f | 1.29E−04 | 3.55 |
| 409038 | T97490 | Hs. 50002 | small inducible cytokine subfamily A (Cy | 7.24E−04 | 3.53 |
| 403809 | | | NM_024743*: Homo sapiens hypothetical pro | 3.37E−02 | 3.52 |
| 417301 | AI478158 | Hs. 164478 | hypothetical protein FLJ21939 similar to | 2.55E−03 | 3.52 |
| 421684 | BE281591 | Hs. 106768 | hypothetical protein FLJ10511 | 4.14E−02 | 3.52 |
| 432834 | F06459 | Hs. 289113 | cytochrome b5 reductase 1 (B5R.1) | 1.69E−02 | 3.52 |
| 439763 | AA845366 | Hs. 184075 | ESTs, Weakly similar to ALU1_HUMAN ALU S | 4.36E−03 | 3.52 |
| 445240 | AI217385 | Hs. 147574 | ESTs | 2.45E−02 | 3.52 |
| 444545 | AW995346 | Hs. 146910 | ESTs | 2.63E−02 | 3.52 |
| 420789 | AI670057 | Hs. 199882 | ESTs | 4.73E−02 | 3.52 |
| 421662 | NM_014141 | Hs. 106552 | cell recognition molecule Caspr2 | 6.19E−04 | 3.51 |
| 426181 | AA371422 | Hs. 334371 | hypothetical protein MGC13096 | 1.59E−03 | 3.51 |
| 421951 | BE327432 | Hs. 109804 | H1 histone family, member X | 1.83E−03 | 3.50 |
| 426780 | BE242284 | Hs. 172199 | adenylate cyclase 7 | 1.67E−03 | 3.50 |
| 429301 | AA449416 | Hs. 31395 | ESTs | 4.76E−03 | 3.50 |
| 430750 | AI650360 | Hs. 100256 | ESTs | 5.89E−03 | 3.50 |
| 443126 | AI033503 | | gb: ox06d11.s1 Soares_fetal_liver_spleen_{—} | 1.39E−02 | 3.50 |
| 450253 | AL133047 | Hs. 24715 | Homo sapiens mRNA; cDNA DKFZp434D0215 (f | 2.79E−03 | 3.50 |
| 442739 | NM_007274 | Hs. 8679 | cytosolic acyl coenzyme A thioester hydr | 1.31E−03 | 3.49 |
| 430478 | NM_014349 | Hs. 241535 | apolipoprotein L, 3 | 1.74E−07 | 3.49 |
| 408784 | AW971350 | Hs. 63386 | ESTs | 1.90E−05 | 3.47 |
| 417562 | AW888754 | Hs. 134126 | crystallin, gamma S | 2.92E−03 | 3.47 |
| 437664 | AW977714 | Hs. 211610 | ESTs, Moderately similar to ALU1_HUMAN A | 3.35E−03 | 3.47 |
| 448219 | AA228092 | | KIAA1681 protein | 1.29E−02 | 3.47 |
| 403671 | | | C4001270*: gi\|7509005\|pir\|\|T26190 hypothe | 8.28E−03 | 3.47 |
| 427008 | Z45258 | Hs. 286013 | short coiled-coil protein | 1.37E−04 | 3.46 |
| 409005 | AW299806 | Hs. 297256 | ESTs | 2.67E−03 | 3.46 |
| 446162 | AI631319 | Hs. 63841 | hypothetical protein DKFZp434E2318 | 1.19E−02 | 3.45 |
| 453686 | AL110326 | Hs. 304679 | ESTs, Moderately similar to Z195_HUMAN Z | 3.66E−04 | 3.45 |
| 441297 | AW403084 | Hs. 7766 | ubiquitin-conjugating enzyme E2E 1 (homo | 8.85E−04 | 3.44 |
| 430457 | AI436216 | Hs. 191715 | ESTs, Weakly similar to ZN91_HUMAN ZINC | 3.66E−03 | 3.43 |
| 413392 | AW021404 | Hs. 13021 | ESTs | 1.29E−02 | 3.42 |
| 416980 | AA381133 | Hs. 80684 | high-mobility group (nonhistone chromoso | 1.45E−02 | 3.42 |
| 417244 | T57053 | Hs. 10136 | ESTs | 4.18E−03 | 3.42 |
| 422343 | AI628633 | Hs. 346823 | gb: ty77d05.x1 NCI_CGAP_Kid11 Homo sapien | 1.06E−02 | 3.42 |
| 426494 | AL119528 | Hs. 170098 | KIAA0372 gene product | 1.44E−02 | 3.42 |
| 427722 | AK000123 | Hs. 180479 | hypothetical protein FLJ20116 | 4.28E−02 | 3.42 |
| 441466 | AW673081 | Hs. 54828 | ESTs | 1.14E−02 | 3.42 |
| 450850 | AA648886 | Hs. 151999 | ESTs | 9.18E−05 | 3.42 |
| 418259 | AA215404 | | ESTs | 3.54E−05 | 3.40 |
| 423032 | AI684746 | Hs. 119274 | RAS p21 protein activator (GTPase activa | 1.25E−03 | 3.40 |
| 452167 | N75238 | Hs. 13075 | Homo sapiens cDNA: FLJ23013 fis, clone L | 4.18E−03 | 3.40 |
| 415023 | AA932146 | | Homo sapiens clone TCCCIA00164 mRNA sequ | 6.18E−04 | 3.40 |
| 424088 | AL049942 | Hs. 139240 | DKFZP564F1422 protein | 8.79E−04 | 3.40 |
| 419438 | AA406400 | Hs. 12482 | glyceronephosphate O-acyltransferase | 7.62E−03 | 3.39 |
| 441077 | AI241273 | Hs. 15312 | ESTs | 3.07E−06 | 3.38 |
| 420664 | AI681270 | Hs. 99824 | BCE-1 protein | 4.09E−04 | 3.38 |
| 426979 | AF161472 | Hs. 173074 | DKFZP564O1863 protein | 7.37E−02 | 3.38 |
| 437410 | AW023340 | Hs. 14880 | ESTs | 4.36E−03 | 3.38 |
| 444430 | AI611153 | Hs. 6093 | Homo sapiens cDNA: FLJ22783 fis, clone K | 4.89E−02 | 3.38 |
| 417678 | X06560 | Hs. 82396 | 2′,5′-oligoadenylate synthetase 1 (40-46 | 4.94E−07 | 3.37 |
| 410243 | D83402 | Hs. 302085 | prostaglandin I2 (prostacyclin) synthase | 4.88E−02 | 3.35 |
| 412634 | U55984 | | heat shock 90 kD protein 1, alpha | 2.51E−04 | 3.35 |
| 419970 | AW612022 | | ESTs | 8.62E−03 | 3.35 |
| 439717 | W94472 | Hs. 59529 | ESTs, Moderately similar to ALU1_HUMAN A | 1.29E−02 | 3.35 |
| 444013 | T08531 | Hs. 44404 | Homo sapiens PRO1488 mRNA, complete cds | 5.91E−03 | 3.35 |
| 444454 | BE018316 | Hs. 11183 | sorting nexin 2 | 1.37E−04 | 3.35 |
| 439776 | AL360140 | Hs. 176005 | Homo sapiens mRNA full length insert cDN | 4.36E−03 | 3.34 |
| 408989 | AW361666 | Hs. 49500 | KIAA0746 protein | 3.99E−05 | 3.34 |
| 452187 | AA400200 | Hs. 19131 | transcription factor Dp-2 (E2F dimerizat | 7.94E−03 | 3.33 |
| 457701 | AW855466 | Hs. 271866 | ESTs, Weakly similar to ALU1_HUMAN ALU S | 7.23E−04 | 3.33 |
| 418318 | U47732 | Hs. 84072 | transmembrane 4 superfamily member 3 | 4.54E−04 | 3.33 |
| 438922 | R71288 | Hs. 259664 | ESTs | 6.44E−03 | 3.33 |
| 430008 | AW085625 | Hs. 186838 | ESTs, Weakly similar to Z295_HUMAN ZINC | 1.06E−02 | 3.33 |
| 438543 | AA810141 | Hs. 192182 | ESTs | 2.16E−05 | 3.32 |
| 447188 | H65423 | Hs. 17631 | hypothetical protein DKFZp434E2135 | 1.29E−02 | 3.32 |
| 454064 | AI130731 | Hs. 57967 | ESTs | 1.10E−02 | 3.32 |
| 433198 | AA992841 | Hs. 27263 | KIAA1458 protein | 3.04E−02 | 3.32 |
| 402041 | | | C15001201*: gi\|6841178\|gb\|AAF28942.1\|AF16 | 3.19E−03 | 3.31 |
| 441028 | AI333660 | Hs. 17558 | Homo sapiens cDNA FLJ14446 fis, clone HE | 9.30E−04 | 3.31 |
| 433483 | AI926520 | Hs. 31016 | putative DNA binding protein | 1.45E−02 | 3.31 |
| 419126 | AI810144 | Hs. 135276 | ESTs | 6.17E−03 | 3.30 |
| 417052 | NM_000712 | Hs. 81029 | biliverdin reductase A | 1.91E−05 | 3.30 |
| 433907 | AW296107 | Hs. 152686 | ESTs | 4.37E−03 | 3.29 |
| 433364 | AI075407 | Hs. 296083 | ESTs, Moderately similar to I54374 gene | 1.75E−07 | 3.29 |
| 449188 | AW072939 | Hs. 347187 | myotubularin related protein 1 | 8.61E−05 | 3.29 |
| 442831 | AI798959 | Hs. 131686 | ESTs | 2.81E−04 | 3.28 |
| 420985 | X94703 | | RAB28, member RAS oncogene family | 1.02E−03 | 3.28 |
| 446111 | W56338 | Hs. 13880 | CGI-143 protein | 1.24E−02 | 3.28 |
| 456984 | AB002349 | | Ral guanine nucleotide exchange factor R | 6.43E−03 | 3.28 |
| 418945 | BE246762 | Hs. 89499 | arachidonate 5-lipoxygenase | 3.66E−04 | 3.27 |
| 427675 | AW138190 | Hs. 180248 | zinc finger protein 124 (HZF-16) | 6.43E−05 | 3.27 |
| 444030 | AW021254 | Hs. 135055 | ESTs | 1.75E−02 | 3.27 |
| 423828 | AA331536 | | gb: EST35377 Embryo, 8 week I Homo sapien | 4.17E−03 | 3.26 |
| 426030 | BE243933 | Hs. 108642 | zinc finger protein 22 (KOX 15) | 5.21E−02 | 3.26 |
| 411777 | BE067552 | | gb: MR4-BT0358-020200-002-g10 BT0358 Homo | 1.59E−03 | 3.26 |
| 413568 | AA130381 | Hs. 180257 | ESTs | 2.16E−05 | 3.25 |
| 421828 | AW891965 | | histone deacetylase 3 | 1.01E−02 | 3.25 |
| 439389 | AA318940 | Hs. 56004 | ESTs | 4.72E−02 | 3.25 |
| 442297 | NM_006202 | Hs. 89901 | phosphodiesterase 4A, cAMP-specific (dun | 3.50E−03 | 3.25 |
| 451099 | R52795 | Hs. 25954 | interleukin 13 receptor, alpha 2 | 1.76E−02 | 3.25 |
| 424915 | R42755 | Hs. 23096 | ESTs | 2.92E−03 | 3.24 |
| 452279 | AA286844 | | hypothetical protein FLJ13164 | 9.93E−02 | 3.24 |
| 418791 | AA935633 | Hs. 194628 | ESTs | 5.92E−03 | 3.24 |
| 453822 | NM_014116 | Hs. 35416 | PRO0132 protein | 7.15E−02 | 3.24 |
| 456119 | AA161411 | Hs. 58668 | chromosome 21 open reading frame 57 | 1.44E−03 | 3.23 |
| 439708 | AI761369 | Hs. 59584 | hypothetical protein FLJ21144 | 1.03E−04 | 3.23 |
| 400189 | | | Eos Control | 1.34E−02 | 3.22 |
| 430929 | AA489166 | Hs. 156933 | ESTs | 4.54E−04 | 3.22 |
| 453128 | AW026516 | Hs. 31791 | acylphosphatase 2, muscle type | 350E−03 | 3.22 |
| 413129 | AF292100 | Hs. 104613 | RP42 homolog | 3.67E−04 | 3.22 |
| 435126 | AI393666 | Hs. 42315 | p10-binding protein | 5.04E−02 | 3.22 |
| 417831 | H16423 | Hs. 82685 | CD47 antigen (Rh-related antigen, integr | 9.76E−04 | 3.21 |
| 449209 | BE616830 | Hs. 294145 | ESTs | 3.05E−03 | 3.20 |
| 414821 | M63835 | Hs. 77424 | Fc fragment of IgG, high affinity la, re | 3.03E−02 | 3.20 |
| 442961 | BE614474 | | F-box only protein 22 | 5.10E−05 | 3.20 |
| 436854 | AA749167 | Hs. 173911 | ESTs | 2.04E−02 | 3.20 |
| 450222 | U75308 | Hs. 24644 | TATA box binding protein (TBP)-associate | 2.32E−03 | 3.20 |
| 426312 | AF026939 | Hs. 181874 | interferon-induced protein with tetratri | 3.66E−07 | 3.18 |
| 418757 | AI864193 | Hs. 169728 | hypothetical protein FLJ13150 | 4.99E−03 | 3.18 |
| 422150 | AI867118 | | calpastatin | 2.83E−02 | 3.18 |
| 403478 | | | NM_022342: Homo sapiens kinesin protein 9 | 6.11E−02 | 3.17 |
| 414650 | AA150435 | Hs. 8248 | ESTs | 5.04E−02 | 3.17 |
| 431188 | W05656 | Hs. 169755 | ESTs | 1.19E−02 | 3.17 |
| 436262 | AA707425 | | gb: ag83f05.r1 Stratagene hNT neuron (937 | 6.44E−03 | 3.17 |
| 439680 | AW245741 | Hs. 58461 | ESTs, Weakly similar to A35659 krueppel- | 3.50E−03 | 3.17 |
| 447371 | AA334274 | Hs. 18368 | DKFZP564B0769 protein | 1.51E−02 | 3.17 |
| 448914 | AI927656 | Hs. 196459 | ESTs | 9.91E−02 | 3.17 |
| 448481 | W15284 | Hs. 74832 | ESTs | 3.35E−03 | 3.17 |
| 414449 | AA557660 | Hs. 76152 | decorin | 3.49E−02 | 3.15 |
| 418721 | NM_002731 | Hs. 87773 | protein kinase, cAMP-dependent, catalyti | 1.71E−04 | 3.15 |
| 407821 | AA346172 | Hs. 195614 | ESTs | 2.65E−04 | 3.15 |
| 410390 | AA876905 | Hs. 125286 | ESTs | 6.72E−03 | 3.15 |
| 412977 | AA125910 | Hs. 191461 | ESTs | 6.51E−04 | 3.15 |
| 426506 | AW935187 | Hs. 170162 | KIAA1357 protein | 2.81E−04 | 3.15 |
| 436169 | AA888311 | Hs. 17602 | Homo sapiens cDNA FLJ 12381 fis, clone MA | 7.20E−04 | 3.15 |
| 441879 | AI521936 | Hs. 107149 | novel protein similar to archaeal, yeast | 1.29E−04 | 3.15 |
| 444342 | NM_014398 | Hs. 10887 | similar to lysosome-associated membrane | 3.41E−07 | 3.15 |
| 448919 | AI590605 | Hs. 191036 | ESTs | 3.04E−03 | 3.15 |
| 408683 | R58665 | Hs. 46847 | TRAF and TNF receptor-associated protein | 2.03E−06 | 3.15 |
| 436535 | AW295687 | Hs. 254420 | ESTs | 1.29E−04 | 3.14 |
| 411060 | NM_006074 | Hs. 318501 | Homo sapiens mRNA full length insert cDN | 2.74E−07 | 3.14 |
| 419195 | AW291165 | Hs. 25447 | ESTs | 4.57E−03 | 3.14 |
| 434821 | AA159111 | Hs. 284281 | Human putative ribosomal protein S1 mRNA | 1.36E−04 | 3.14 |
| 450056 | BE047394 | Hs. 8208 | ESTs, Weakly similar to S71512 hypotheti | 2.66E−04 | 3.13 |
| 429286 | AA449239 | Hs. 154855 | ESTs | 1.13E−03 | 3.13 |
| 418027 | AB037807 | Hs. 83293 | hypothetical protein | 6.08E−05 | 3.13 |
| 450374 | AA397540 | Hs. 60293 | Homo sapiens clone 122482 unknown mRNA | 5.05E−02 | 3.13 |
| 426381 | T61692 | Hs. 269340 | ESTs | 7.37E−02 | 3.12 |
| 413838 | AV661185 | Hs. 75574 | mitochondrial ribosomal protein L19 | 2.43E−03 | 3.12 |
| 408931 | AA251995 | Hs. 334648 | poly(A) polymerase alpha | 4.97E−03 | 3.12 |
| 430532 | D61216 | Hs. 18672 | ESTs | 4.77E−03 | 3.12 |
| 446177 | AK001902 | Hs. 14202 | hypothetical protein FLJ11040 | 1.80E−04 | 3.11 |
| 407436 | AF211977 | | gb: Homo sapiens LENG10 mRNA, partial seq | 1.63E−02 | 3.11 |
| 418733 | AA227714 | | KIAA0129 gene product | 2.03E−06 | 3.11 |
| 403027 | | | C21000364*: gi\|8394509\|ref\|NP_058778.1\|u | 8.80E−04 | 3.11 |
| 419257 | X53461 | Hs. 89781 | upstream binding transcription factor, R | 1.15E−04 | 3.11 |
| 410425 | BE278367 | Hs. 63510 | KIAA0141 gene product | 1.63E−02 | 3.10 |
| 426647 | AA243464 | Hs. 294101 | pre-B-cell leukemia transcription factor | 1.92E−03 | 3.10 |
| 434629 | AA789081 | Hs. 4029 | glioma-amplified sequence-41 | 2.12E−03 | 3.10 |
| 418182 | AW016405 | Hs. 16648 | ESTs | 1.67E−03 | 3.09 |
| 449832 | AA694264 | Hs. 60049 | ESTs | 2.73E−02 | 3.09 |
| 407813 | AL120247 | Hs. 40109 | KIAA0872 protein | 2.26E−04 | 3.08 |
| 452664 | AA398859 | Hs. 18397 | hypothetical protein FLJ23221 | 3.07E−06 | 3.08 |
| 410851 | AW612147 | Hs. 32058 | Homo sapiens C1orf19 mRNA, partial cds | 2.79E−03 | 3.07 |
| 448362 | AA641767 | Hs. 21015 | hypothetical protein DKFZp564L0864 simil | 4.14E−02 | 3.07 |
| 449656 | AA002008 | Hs. 188633 | ESTs | 2.02E−03 | 3.07 |
| 416039 | AA376989 | Hs. 78989 | alcohol dehydrogenase 5 (class III), chi | 1.03E−03 | 3.07 |
| 418729 | AB028449 | Hs. 87889 | helicase-moi | 2.52E−04 | 3.07 |
| 431629 | AU077025 | Hs. 265827 | interferon, alpha-inducible protein (clo | 2.74E−07 | 3.06 |
| 437151 | AA745618 | | BANP homolog, SMAR1 homolog | 5.93E−02 | 3.06 |
| 414948 | C15240 | Hs. 182155 | ESTs | 8.63E−05 | 3.05 |
| 419465 | AW500239 | Hs. 21187 | Homo sapiens cDNA: FLJ23068 fis, clone L | 2.63E−02 | 3.05 |
| 432680 | T47364 | Hs. 278613 | interferon, alpha-inducible protein 27 | 1.75E−07 | 3.05 |
| 418627 | AL079835 | Hs. 86858 | ribosomal protein S6 kinase, 70 kD, polyp | 1.02E−03 | 3.04 |
| 408753 | AI337192 | Hs. 47438 | SH3 domain binding glutamic acid-rich pr | 1.75E−02 | 3.04 |
| 421654 | AW163267 | Hs. 106469 | suppressor of var1 (S. cerevisiae) 3-like | 6.12E−02 | 3.03 |
| 421272 | AA704157 | | ESTs | 7.22E−04 | 3.03 |
| 420174 | AI824144 | Hs. 199749 | ESTs | 4.79E−05 | 3.03 |
| 439024 | R96696 | Hs. 35598 | ESTs | 3.61E−02 | 3.03 |
| 407705 | AB023139 | Hs. 37892 | KIAA0922 protein | 4.29E−04 | 3.02 |
| 409977 | AW805510 | Hs. 97056 | hypothetical protein FLJ21634 | 5.58E−04 | 3.02 |
| 411960 | R77776 | Hs. 18103 | ESTs | 1.05E−02 | 3.02 |
| 437086 | AW291411 | Hs. 192531 | ESTs, Weakly similar to S00754 zinc fing | 5.20E−03 | 3.02 |
| 442071 | BE048433 | Hs. 276043 | ESTs | 2.02E−03 | 3.02 |
| 448030 | N30714 | Hs. 325960 | membrane-spanning 4-domains, subfamily A | 4.87E−02 | 3.02 |
| 430068 | AA464964 | | gb: zx80f10.s1 Soares ovary tumor NbHOT H | 9.30E−04 | 3.02 |
| 436090 | AI640635 | Hs. 332879 | EST | 2.04E−02 | 3.01 |
| 408047 | AW205461 | Hs. 243612 | ESTs | 1.81E−04 | 3.00 |
| 412288 | NM_003005 | Hs. 73800 | selectin P (granule membrane protein 140 | 5.91E−03 | 3.00 |
| 432474 | AA584042 | | gb: nn65e09.s1 NCI_CGAP_Lar1 Homo sapiens | 6.72E−02 | 3.00 |
| 446851 | AW007332 | | Homo sapiens cDNA: FLJ22063 fis, clone H | 2.43E−03 | 3.00 |
| 451171 | AA248829 | Hs. 112921 | gb: jj6059.seq.F Human fetal heart, Lambd | 7.98E−04 | 3.00 |
| 452429 | AK000149 | Hs. 29493 | hypothetical protein FLJ20142 | 8.96E−03 | 3.00 |
| 413278 | BE563085 | Hs. 833 | interferon-stimulated protein, 15 kDa | 1.75E−07 | 2.98 |
| 426221 | AB007881 | | KIAA0421 protein | 2.55E−03 | 2.98 |
| 411190 | AA306342 | Hs. 69171 | protein kinase C-like 2 | 3.87E−02 | 2.97 |
| 444437 | AI377961 | Hs. 44041 | ESTs | 8.62E−03 | 2.97 |
| 451572 | AA018556 | Hs. 268691 | ESTs, Moderately similar to ALU2_HUMAN A | 1.09E−04 | 2.97 |
| 452420 | BE564871 | Hs. 29463 | centrin, EF-hand protein, 3 (CDC31 yeast | 6.84E−05 | 2.97 |
| 412675 | AA460716 | Hs. 9788 | hypothetical protein MGC10924 similar to | 2.04E−02 | 2.97 |
| 446215 | AW821329 | Hs. 14368 | SH3 domain binding glutamic acid-rich pr | 6.45E−03 | 2.96 |
| 442287 | AW952703 | Hs. 8182 | synaptic nuclei expressed gene 1b | 1.67E−03 | 2.96 |
| 439658 | AA332057 | Hs. 6639 | hypothetical protein MGC15440 | 5.03E−04 | 2.96 |
| 400471 | | | Target Exon | 6.12E−02 | 2.95 |
| 431266 | AW149321 | Hs. 105411 | ESTs | 2.12E−03 | 2.95 |
| 404433 | | | C8000067*: gi\|10432400\|emb\|CAC10290.1\|(A | 1.75E−02 | 2.95 |
| 435852 | H72303 | Hs. 36011 | ESTs | 4.18E−03 | 2.95 |
| 437275 | AW976035 | Hs. 292396 | ESTs, Weakly similar to A47582 B-cell gr | 4.18E−03 | 2.95 |
| 439601 | AB029032 | Hs. 6606 | KIAA1109 protein | 1.50E−02 | 2.95 |
| 458455 | AV648310 | Hs. 213488 | ESTs | 8.62E−03 | 2.95 |
| 411943 | BE502436 | Hs. 7962 | ESTs, Weakly similar to S44608 C02F5.6 p | 7.94E−03 | 2.95 |
| 421718 | AL117574 | Hs. 193602 | Homo sapiens mRNA; cDNA DKFZp434L2221 (f | 1.71E−04 | 2.94 |
| 450256 | AA286887 | Hs. 24724 | MFH-amplified sequences with leucine-ric | 7.26E−05 | 2.94 |
| 401898 | | | NM_024722*: Homo sapiens hypothetical pro | 4.00E−03 | 2.94 |
| 458368 | BE504731 | Hs. 138827 | ESTs | 2.92E−03 | 2.94 |
| 456563 | AA989220 | Hs. 766 | ESTs | 1.51E−02 | 2.93 |
| 402836 | | | ENSP00000251163*: Membrane-associated gua | 3.86E−02 | 2.92 |
| 409444 | H47933 | Hs. 33983 | ESTs, Weakly similar to ALU6_HUMAN ALU S | 1.08E−04 | 2.92 |
| 418522 | AA605038 | Hs. 7149 | Homo sapiens cDNA: FLJ21950 fis, clone H | 4.30E−04 | 2.92 |
| 423129 | L44396 | Hs. 124106 | Homo sapiens cDNA FLJ11941 fis, clone HE | 4.77E−03 | 2.92 |
| 426279 | AI648520 | Hs. 169084 | tubby like protein 3 | 1.75E−02 | 2.92 |
| 441969 | AI733386 | | ESTs, Weakly similar to ALU1_HUMAN ALU S | 7.20E−04 | 2.92 |
| 446552 | AW470827 | Hs. 156241 | ESTs | 2.02E−04 | 2.92 |
| 451644 | N23235 | Hs. 30567 | ESTs, Weakly similar to B34087 hypotheti | 2.25E−04 | 2.92 |
| 403330 | | | Target Exon | 7.23E−04 | 2.92 |
| 414617 | AI339520 | Hs. 288817 | ESTs, Moderately similar to N Chain N, M | 3.77E−05 | 2.92 |
| 459436 | AA323121 | | gb: EST25881 Cerebellum II Homo sapiens c | 6.52E−04 | 2.91 |
| 420181 | AI380089 | Hs. 158951 | ESTs | 7.30E−03 | 2.91 |
| 447002 | BE242866 | Hs. 16933 | HepA-related protein | 2.49E−06 | 2.91 |
| 408212 | AA297567 | Hs. 43728 | hypothetical protein | 1.19E−03 | 2.91 |
| 417008 | AA191708 | Hs. 325825 | Homo sapiens cDNA FLJ20848 fis, clone AD | 1.52E−03 | 2.90 |
| 434263 | N34895 | Hs. 79187 | ESTs | 7.62E−03 | 2.90 |
| 403743 | | | C1002604: gi\|8393668\|ref\|NP 058989.1\|kin | 7.94E−03 | 2.90 |
| 414429 | R51494 | Hs. 71818 | ESTs | 9.10E−02 | 2.90 |
| 420265 | AA766209 | Hs. 88087 | ESTs | 2.93E−02 | 2.90 |
| 420683 | AA830168 | Hs. 271305 | ESTs | 1.06E−02 | 2.90 |
| 426590 | AA617830 | Hs. 28310 | ESTs | 2.02E−03 | 2.90 |
| 428738 | NM_000380 | Hs. 192803 | xeroderma pigmentosum, complementation g | 1.25E−03 | 2.90 |
| 437108 | AA434054 | Hs. 80624 | hypothetical protein MGC2560 | 1.75E−02 | 2.90 |
| 451078 | AI927694 | Hs. 340945 | ESTs | 8.83E−02 | 2.90 |
| 442439 | U09759 | Hs. 246857 | mitogen-activated protein kinase 9 | 7.24E−05 | 2.90 |
| 409939 | AA463437 | Hs. 11556 | Homo sapiens cDNA FLJ12566 fis, clone NT | 2.52E−04 | 2.90 |
| 414493 | AL133921 | Hs. 76272 | retinoblastoma-binding protein 2 | 1.58E−05 | 2.90 |
| 434045 | AI065133 | Hs. 152316 | hypothetical protein PRO0971 | 9.77E−04 | 2.90 |
| 440668 | AI989538 | Hs. 191074 | ESTs | 2.22E−03 | 2.89 |
| 446217 | AI651594 | Hs. 99709 | ESTs | 4.26E−07 | 2.89 |
| 438914 | N93892 | Hs. 10727 | ESTs | 2.11E−02 | 2.89 |
| 443303 | U67319 | Hs. 9216 | caspase 7, apoptosis-related cysteine pr | 1.81E−04 | 2.89 |
| 418584 | NM_004606 | Hs. 1179 | TATA box binding protein (TBP)-associate | 3.66E−03 | 2.88 |
| 428695 | AI355647 | Hs. 189999 | purinergic receptor (family A group 5) | 4.24E−05 | 2.88 |
| 436372 | AW972301 | Hs. 310286 | ESTs | 1.03E−04 | 2.87 |
| 411360 | AK001601 | Hs. 69594 | high-mobility group 20A | 3.61E−02 | 2.87 |
| 437456 | AL047045 | Hs. 60293 | Homo sapiens clone 122482 unknown mRNA | 2.55E−03 | 2.86 |
| 448198 | BE622100 | Hs. 209406 | ESTs, Weakly similar to I38600 zinc fing | 5.39E−05 | 2.86 |
| 428974 | AA442693 | Hs. 272006 | ESTs, Weakly similar to I38022 hypotheti | 3.33E−05 | 2.86 |
| 423568 | NM_005256 | Hs. 129818 | growth arrest-specific 2 | 5.04E−04 | 2.85 |
| 418796 | AA228351 | Hs. 34060 | ESTs | 2.79E−04 | 2.85 |
| 406274 | | | Target Exon | 4.78E−04 | 2.85 |
| 427209 | H06509 | Hs. 92423 | KIAA1566 protein | 2.61E−05 | 2.85 |
| 430280 | AA361258 | Hs. 237868 | interleukin 7 receptor | 2.11E−03 | 2.85 |
| 440760 | AK001145 | Hs. 284216 | hypothetical protein FLJ10283 | 3.19E−03 | 2.85 |
| 415914 | AA306033 | Hs. 78915 | GA-binding protein transcription factor, | 7.37E−02 | 2.85 |
| 448569 | BE382657 | Hs. 21486 | signal transducer and activator of trans | 1.75E−07 | 2.84 |
| 425284 | AF155568 | | NS1-associated protein 1 | 1.59E−03 | 2.84 |
| 422614 | AI908006 | Hs. 295362 | Homo sapiens cDNA FLJ14459 fis, clone HE | 6.44E−03 | 2.84 |
| 424755 | AB033094 | Hs. 152925 | KIAA1268 protein | 1.08E−06 | 2.83 |
| 411617 | AA247994 | | neurocalcin delta | 2.63E−02 | 2.83 |
| 452699 | AW295390 | Hs. 213062 | ESTs | 8.27E−03 | 2.83 |
| 401197 | | | ENSP00000229263*: HSPC213. | 4.72E−02 | 2.82 |
| 409205 | AI952884 | Hs. 14832 | ESTs, Moderately similar to unnamed prot | 1.06E−02 | 2.82 |
| 429503 | AA394183 | Hs. 204166 | ESTs | 3.50E−03 | 2.82 |
| 436114 | AA778232 | Hs. 19515 | ESTs, Highly similar to NRG3_HUMAN PRO-N | 8.26E−03 | 2.82 |
| 445564 | AB028957 | Hs. 12896 | KIAA1034 protein | 1.50E−02 | 2.82 |
| 443280 | AA299688 | Hs. 24183 | ESTs | 2.55E−03 | 2.82 |
| 410541 | AA065003 | Hs. 64179 | syntenin-2 protein | 2.55E−03 | 2.82 |
| 439653 | AW021103 | Hs. 6631 | hypothetical protein FLJ20373 | 6.73E−03 | 2.82 |
| 446238 | T95143 | Hs. 14511 | SCO (cytochrome oxidase deficient, yeast | 3.13E−04 | 2.82 |
| 428418 | AI368826 | Hs. 8768 | ESTs | 6.46E−05 | 2.81 |
| 431392 | AI371223 | Hs. 288671 | Homo sapiens cDNA FLJ11997 fis, clone HE | 6.18E−03 | 2.81 |
| 434519 | AA635727 | Hs. 136581 | ESTs, Weakly similar to ARNO_HUMAN ARF N | 2.32E−03 | 2.80 |
| 430027 | AB023197 | Hs. 227743 | KIAA0980 protein | 1.90E−06 | 2.80 |
| 425836 | AW955696 | Hs. 90960 | ESTs | 4.78E−05 | 2.80 |
| 428821 | H91282 | Hs. 286232 | Homo sapiens cDNA: FLJ23190 fis, clone L | 8.62E−03 | 2.80 |
| 439773 | AI051313 | Hs. 143315 | ESTs | 5.41E−03 | 2.80 |
| 432485 | N90866 | Hs. 276770 | CDW52 antigen (CAMPATH-1 antigen) | 1.01E−06 | 2.80 |
| 456508 | AA502764 | Hs. 123469 | ESTs, Weakly similar to AF208855 1 BM-01 | 4.98E−03 | 2.79 |
| 436235 | AI084982 | Hs. 120790 | ESTs | 8.15E−05 | 2.79 |
| 421446 | AA682425 | Hs. 118959 | ESTs | 1.34E−02 | 2.78 |
| 421524 | AA312082 | Hs. 105445 | GDNF family receptor alpha 1 | 1.69E−05 | 2.77 |
| 421743 | T35958 | Hs. 107614 | DKFZP564I1171 protein | 3.48E−04 | 2.76 |
| 404676 | | | Target Exon | 7.31E−03 | 2.75 |
| 411352 | NM_002890 | Hs. 758 | RAS p21 protein activator (GTPase activa | 1.59E−03 | 2.75 |
| 424381 | AA285249 | Hs. 146329 | protein kinase Chk2 (CHEK2) | 2.54E−02 | 2.75 |
| 426416 | AW612744 | Hs. 169824 | killer cell lectin-like receptor subfami | 2.18E−02 | 2.75 |
| 432134 | AI816782 | Hs. 122583 | hypothetical protein FLJ21934 | 1.84E−03 | 2.75 |
| 434521 | NM_002267 | Hs. 3886 | karyopherin alpha 3 (importin alpha 4) | 221E−03 | 2.75 |
| 434963 | AW974957 | Hs. 288719 | Homo sapiens cDNA FLJ12142 fis, clone MA | 2.63E−02 | 2.75 |
| 436184 | BE154067 | Hs. 136660 | ESTs, Weakly similar to ZN91_HUMAN ZINC | 3.20E−03 | 2.75 |
| 439605 | AF086431 | Hs. 22380 | ESTs | 8.61E−05 | 2.75 |
| 449259 | AW452058 | Hs. 257519 | ESTs | 3.66E−04 | 2.75 |
| 452057 | AW952005 | Hs. 14928 | hypothetical protein FLJ12903 | 3.83E−03 | 2.75 |
| 422231 | AA443512 | Hs. 101383 | ESTs | 1.84E−03 | 2.75 |
| 418941 | AA452970 | Hs. 239527 | E1B-55 kDa-associated protein 5 | 5.56E−02 | 2.74 |
| 424637 | NM_015057 | Hs. 151411 | KIAA0916 protein | 8.30E−06 | 2.74 |
| 436741 | AA860163 | Hs. 291319 | ESTs | 2.79E−03 | 2.72 |
| 439462 | AL133026 | Hs. 6567 | Homo sapiens mRNA; cDNA DKF2p434C136 (fr | 1.31E−03 | 2.72 |
| 414522 | AW518944 | Hs. 76325 | Immunoglobulin J chain | 2.12E−03 | 2.72 |
| 426125 | X87241 | Hs. 166994 | FAT tumor suppressor (Drosophila) homolo | 6.94E−02 | 2.72 |
| 427297 | AW292593 | Hs. 334907 | Homo sapiens, clone MGC: 17333, mRNA, com | 2.02E−04 | 2.72 |
| 423067 | AA321355 | Hs. 285401 | colony stimulating factor 2 receptor, be | 6.17E−03 | 2.72 |
| 444745 | AF117754 | Hs. 11861 | thyroid hormone receptor-associated prot | 9.77E−04 | 2.72 |
| 420962 | NM_005904 | Hs. 100602 | MAD (mothers against decapentaplegic, Dr | 1.68E−05 | 2.72 |
| 435427 | AA682573 | Hs. 188982 | ESTs, Weakly similar to organic anion tr | 1.67E−03 | 2.71 |
| 453779 | N35187 | Hs. 43388 | 28 kD interferon responsive protein | 3.17E−07 | 2.71 |
| 439559 | AW364675 | Hs. 173921 | ESTs, Weakly similar to 2109260A B cell | 1.25E−03 | 2.71 |
| 451119 | AA805417 | Hs. 64753 | ESTs | 7.60E−03 | 2.70 |
| 420825 | AI656727 | Hs. 194657 | gb: tt53f12.x1 NCI_CGAP_GC6 Homo sapiens | 5.58E−04 | 2.70 |
| 440538 | W76332 | Hs. 79107 | mitogen-activated protein kinase 14 | 1.45E−02 | 2.70 |
| 407796 | AA195509 | Hs. 39733 | postsynaptic protein CRIPT | 3.49E−03 | 2.70 |
| 401635 | | | C11000702: gi\|10048448\|ref\|NP_065258.1\|g | 1.24E−02 | 2.70 |
| 408051 | AI623351 | Hs. 172148 | ESTs | 3.04E−02 | 2.70 |
| 411213 | AA676939 | Hs. 69285 | neuropilin 1 | 1.45E−02 | 2.70 |
| 412935 | BE267045 | Hs. 75064 | tubulin-specific chaperone c | 1.49E−05 | 2.70 |
| 416048 | H16268 | | gb: ym22a06.r1 Soares infant brain 1NIB H | 4.07E−04 | 2.70 |
| 445715 | AB012958 | Hs. 13137 | UV radiation resistance associated gene | 3.48E−04 | 2.70 |
| 437145 | AF007216 | Hs. 5462 | solute carrier family 4, sodium bicarbon | 1.29E−02 | 2.69 |
| 414404 | W16712 | | KIAA0306 protein | 3.99E−05 | 2.68 |
| 420058 | AK001423 | Hs. 94694 | Homo sapiens cDNA FLJ10561 fis, clone NT | 1.91E−05 | 2.68 |
| 417558 | AF045229 | Hs. 82280 | regulator of G-protein signalling 10 | 2.54E−02 | 2.68 |
| 420339 | AW968259 | Hs. 186647 | ESTs | 1.62E−04 | 2.68 |
| 419497 | NM_006410 | Hs. 90753 | Tat-interacting protein (30 kD) | 1.51E−03 | 2.67 |
| 426797 | AW936258 | Hs. 342849 | ADP-ribosylation factor-like 5 | 8.07E−02 | 2.67 |
| 430594 | AK000790 | Hs. 246885 | hypothetical protein FLJ20783 | 4.88E−02 | 2.67 |
| 433312 | AI241331 | Hs. 131765 | ESTs, Moderately similar to I38937 DNA/R | 7.59E−04 | 2.67 |
| 437838 | AI307229 | | ESTs | 1.19E−03 | 2.67 |
| 438182 | AW342140 | Hs. 182545 | ESTs, Weakly similar to ALU1_HUMAN ALU S | 7.69E−05 | 2.67 |
| 450468 | AW379075 | Hs. 141742 | Homo sapiens cDNA FLJ12211 fis, clone MA | 5.67E−03 | 2.67 |
| 431315 | AW972227 | Hs. 163986 | Homo sapiens cDNA: FLJ22765 fis, clone K | 2.81E−04 | 2.67 |
| 425548 | AA890023 | Hs. 1906 | prolactin receptor | 7.58E−04 | 2.67 |
| 434669 | AF151534 | Hs92023 | core histone macroH2A2.2 | 6.01E−06 | 2.67 |
| 409600 | AJ011679 | Hs. 55099 | rab6 GTPase activating protein (GAP and | 1.08E−04 | 2.66 |
| 417928 | AA209344 | Hs. 30177 | ESTs | 1.81E−04 | 2.66 |
| 418832 | X04011 | Hs. 88974 | cytochrome b-245, beta polypeptide (chro | 2.52E−04 | 2.66 |
| 452696 | AI826645 | Hs. 211534 | ESTs | 2.45E−05 | 2.66 |
| 426126 | AL118747 | Hs. 26691 | ESTs | 6.18E−04 | 2.66 |
| 427528 | AU077143 | Hs. 179565 | minichromosome maintenance deficient (S. | 2.45E−05 | 2.65 |
| 434384 | AA631910 | | ESTs | 2.79E−03 | 2.65 |
| 440529 | AW207640 | Hs. 16478 | Homo sapiens cDNA: FLJ21718 fis, clone C | 8.62E−03 | 2.65 |
| 446830 | BE179030 | | Human DNA sequence from clone RP5-1174N9 | 6.85E−04 | 2.65 |
| 443405 | AF031463 | Hs. 9302 | phosducin-like | 2.65E−04 | 2.65 |
| 407807 | AL031427 | Hs. 40094 | Human DNA sequence from clone 167A19 on | 2.43E−03 | 2.65 |
| 421919 | AJ224901 | Hs. 109526 | zinc finger protein 198 | 1.38E−03 | 2.64 |
| 429617 | X89984 | Hs. 211563 | B-cell CLL/lymphoma 7A | 3.65E−03 | 2.63 |
| 403976 | | | Target Exon | 2.43E−03 | 2.63 |
| 435631 | BE254086 | Hs. 29647 | uncharacterized hematopoietic stem/proge | 7.30E−03 | 2.63 |
| 447657 | AI953011 | | ESTs | 1.52E−03 | 2.63 |
| 412520 | AA442324 | Hs. 795 | H2A histone family, member O | 5.74E−05 | 2.62 |
| 417317 | AW296584 | Hs. 293782 | ESTs | 5.71E−05 | 2.62 |
| 450331 | AA009536 | Hs. 38323 | ESTs | 1.10E−02 | 2.61 |
| 438441 | AW664960 | Hs. 205319 | ESTs | 2.26E−04 | 2.61 |
| 413836 | W92003 | Hs. 70614 | ESTs | 1.24E−02 | 2.61 |
| 446006 | NM_004403 | Hs. 13530 | deafness, autosomal dominant 5 | 1.45E−02 | 2.60 |
| 431214 | AA294921 | Hs. 348024 | v-ral simian leukemia viral oncogene horn | 1.39E−02 | 2.60 |
| 422283 | AW411307 | Hs. 114311 | CDC45 (cell division cycle 45, S.cerevis | 1.08E−04 | 2.60 |
| 425118 | AU076611 | Hs. 154672 | methylene tetrahydrofolate dehydrogenase | 9.36E−03 | 2.60 |
| 442432 | BE093589 | Hs. 38178 | hypothetical protein FLJ23468 | 6.43E−05 | 2.60 |
| 452014 | AI828174 | Hs. 227049 | ESTs | 1.39E−02 | 2.60 |
| 408138 | AA535740 | | tumor protein p53-binding protein, 1 | 1.75E−02 | 2.60 |
| 457819 | AA057484 | Hs. 35406 | ESTs, Highly similar to unnamed protein | 2.27E−02 | 2.60 |
| 451578 | NM_016323 | Hs. 26663 | cyclin-E binding protein 1 | 1.16E−06 | 2.59 |
| 428330 | L22524 | Hs. 2256 | matrix metalloproteinase 7 (matrilysin, | 1.51E−02 | 2.59 |
| 444057 | AA316896 | Hs. 257267 | FYVE and coiled-coil domain containing 1 | 2.26E−04 | 2.58 |
| 401928 | | | Target Exon | 7.00E−03 | 2.57 |
| 424375 | AF070547 | Hs. 146312 | Homo sapiens clone 24820 mRNA sequence | 3.87E−02 | 2.57 |
| 445106 | T10219 | Hs. 12329 | KIAA0697 protein | 7.60E−04 | 2.57 |
| 447211 | AL161961 | Hs. 17767 | KIAA1554 protein | 3.68E−07 | 2.57 |
| 427704 | AW971063 | Hs. 292882 | ESTs | 7.84E−02 | 2.56 |
| 430399 | AI916284 | Hs. 199671 | ESTs | 5.58E−04 | 2.56 |
| 422461 | NM_003417 | Hs. 117077 | zinc finger protein 264 | 1.30E−03 | 2.56 |
| 401952 | | | Target Exon | 1.05E−02 | 2.56 |
| 422392 | NM_005908 | Hs. 115945 | mannosidase, beta A, lysosomal | 7.14E−02 | 2.56 |
| 422722 | H74219 | Hs. 269772 | ESTs | 1.44E−03 | 2.56 |
| 414279 | AW021691 | | GCN5 (general control of amino-acid synt | 9.09E−02 | 2.55 |
| 446934 | AK001943 | Hs. 16577 | F-box only protein 3 | 5.20E−03 | 2.55 |
| 434948 | AI498469 | Hs. 12622 | ESTs, Highly similar to AF161436 1 HSPC3 | 2.73E−02 | 2.55 |
| 438865 | H64256 | Hs. 167619 | ESTs, Moderately similar to ALUC_HUMAN! | 8.08E−02 | 2.55 |
| 439372 | AF088033 | Hs. 159225 | ESTs | 3.25E−02 | 2.55 |
| 445757 | AW449065 | Hs. 13264 | KIAA0856 protein | 3.35E−03 | 2.55 |
| 409614 | BE297412 | Hs. 55189 | hypothetical protein | 2.74E−07 | 2.55 |
| 423932 | T95633 | Hs. 189703 | ESTs | 4.73E−02 | 2.55 |
| 451743 | AW074266 | Hs. 23071 | ESTs | 8.16E−05 | 2.54 |
| 416272 | AA178882 | | gb: zp38b09.r1 Stratagene muscle 937209 H | 1.29E−02 | 2.53 |
| 446161 | AA628206 | Hs. 14125 | p53 regulated PA26 nuclear protein | 1.45E−03 | 2.53 |
| 425303 | AA354785 | | gb: EST63098 Jurkat T-cells V Homo sapien | 2.04E−02 | 2.53 |
| 451253 | H48299 | Hs. 26126 | claudin 10 | 1.38E−03 | 2.52 |
| 408340 | AB037762 | Hs. 44268 | myelin gene expression factor 2 | 6.42E−05 | 2.52 |
| 410678 | BE540516 | Hs. 293732 | hypothetical protein MGC3195 | 1.31E−03 | 2.52 |
| 414792 | BE314949 | Hs. 87128 | hypothetical protein FLJ23309 | 4.23E−06 | 2.52 |
| 447387 | AI268331 | Hs. 102237 | tubby super-family protein | 3.51E−06 | 2.52 |
| 407765 | AW076027 | Hs. 257711 | ESTs, Moderately similar to ALU8_HUMAN A | 3.49E−02 | 2.51 |
| 421471 | U90545 | Hs. 327179 | solute carrier family 17 (sodium phospha | 2.19E−02 | 2.51 |
| 438980 | AW502384 | | gb: UI-HF-BR0p-aka-f-12-0-UI.r1 NIH_MGC_5 | 1.39E−02 | 2.51 |
| 453906 | AW444952 | Hs. 257054 | ESTs | 3.83E−03 | 2.51 |
| 414760 | BE298063 | Hs. 77254 | chromobox homolog 1 (Drosophila HP1 beta | 8.78E−07 | 2.51 |
| 434822 | AW076088 | Hs. 4187 | hypothetical protein 24636 | 2.38E−04 | 2.51 |
| 458079 | AI796870 | Hs. 54277 | DNA segment on chromosome X (unique) 992 | 1.15E−05 | 2.50 |
| 408521 | AA055264 | Hs. 260848 | ESTs, Weakly similar to S23650 retroviru | 1.39E−05 | 2.50 |
| 409161 | W07662 | Hs. 50861 | sirtuin (silent mating type information | 5.39E−05 | 2.50 |
| 411931 | AW675180 | Hs. 36828 | ESTs | 2.28E−02 | 2.50 |
| 414172 | AW954324 | Hs. 75790 | phosphatidylinositol glycan, class C | 4.17E−03 | 2.50 |
| 414502 | AL133721 | Hs. 224680 | ESTs | 6.44E−03 | 2.50 |
| 420613 | AI873871 | Hs. 7041 | ESTs, Weakly similar to A47582 B-cell gr | 4.60E−06 | 2.50 |
| 422195 | AB007903 | Hs. 113082 | KIAA0443 gene product | 1.89E−02 | 2.50 |
| 423645 | AI215632 | Hs. 147487 | ESTs | 1.62E−04 | 2.50 |
| 427210 | BE396283 | Hs. 173987 | eukaryotic translation initiation factor | 1.96E−02 | 2.50 |
| 430273 | AI311127 | Hs. 125522 | ESTs | 1.25E−03 | 2.50 |
| 445664 | AW968638 | Hs. 237691 | ESTs, Weakly similar to KIAA0601 protein | 4.30E−04 | 2.50 |
| 437708 | AB033020 | Hs. 5801 | KIAA1194 protein | 8.63E−05 | 2.50 |
| 439748 | AL389934 | Hs. 23248 | hypothetical protein from EUROIMAGE 2005 | 6.71E−03 | 2.50 |
| 435727 | T78475 | Hs. 269542 | ESTs, Weakly similar to S65657 alpha-1C- | 1.19E−02 | 2.49 |
| 414602 | AW630088 | Hs. 76550 | Homo sapiens mRNA; cDNA DKFZp564B1264 (f | 7.23E−04 | 2.49 |
| 451693 | BE220445 | Hs. 279635 | ESTs | 1.29E−02 | 2.49 |
| 443849 | BE566066 | Hs. 9893 | ASB-3 protein | 2.79E−03 | 2.48 |
| 421405 | AA251944 | Hs. 104058 | CGI-29 protein | 7.60E−04 | 2.48 |
| 446591 | H44186 | Hs. 15456 | PDZ domain containing 1 | 2.68E−06 | 2.48 |
| 432348 | AA534353 | Hs. 194081 | ESTs, Weakly similar to I38022 hypotheti | 4.76E−03 | 2.48 |
| 415323 | BE269352 | Hs. 949 | neutrophil cytosolic factor 2 (65 kD, chr | 2.73E−02 | 2.48 |
| 416436 | H55746 | Hs. 28704 | ESTs, Weakly similar to A45910 ultra-hig | 2.20E−02 | 2.48 |
| 446044 | H67567 | Hs. 13572 | calcium modulating ligand | 2.03E−06 | 2.48 |
| 446771 | AA128965 | Hs. 60679 | TATA box binding protein (TBP)-associate | 7.27E−05 | 2.48 |
| 407112 | AA070801 | Hs. 51615 | ESTs, Weakly similar to ALU7_HUMAN ALU S | 7.60E−02 | 2.47 |
| 410196 | AI936442 | Hs. 59838 | hypothetical protein FLJ10808 | 3.30E−04 | 2.47 |
| 427254 | AL121523 | Hs. 97774 | ESTs | 9.75E−03 | 2.47 |
| 430487 | D87742 | Hs. 241552 | KIAA0268 protein | 7.98E−04 | 2.47 |
| 452480 | AI903526 | | gb: RC-BT031-090199-063 BT031 Homo sapien | 1.51E−02 | 2.47 |
| 422473 | U94780 | Hs. 117242 | meningioma expressed antigen 6 (coiled-c | 7.00E−03 | 2.47 |
| 405117 | | | C11000181*: gi\|7305349\|ref\|NP_038647.1\|o | 1.37E−03 | 2.47 |
| 449082 | BE387561 | Hs. 22981 | DKFZP586M1523 protein | 6.16E−04 | 2.47 |
| 439815 | AA206079 | Hs. 6693 | hypothetical protein FLJ20420 | 2.02E−04 | 2.47 |
| 408145 | AF182316 | Hs. 234680 | fer-1 (C. elegans)-like 3 (myofertin) | 5.38E−02 | 2.47 |
| 448122 | AW665656 | Hs. 173187 | ESTs | 5.92E−02 | 2.47 |
| 414895 | AW894856 | Hs. 116278 | Homo sapiens cDNA FLJ13571 fis, clone PL | 3.82E−03 | 2.46 |
| 439747 | AK001148 | Hs. 6671 | COP9 complex subunit 4 | 6.85E−04 | 2.46 |
| 440706 | AA927562 | Hs. 148234 | ESTs | 2.79E−03 | 2.46 |
| 400557 | | | Target Exon | 3.14E−02 | 2.46 |
| 456721 | AA533356 | | gb: nj67f10.s1 NCI_CGAP_Pr10 Homo sapiens | 4.00E−03 | 2.45 |
| 416354 | NM_000633 | Hs. 79241 | B-cell CLL/lymphoma 2 (BCL2) | 5.04E−04 | 2.45 |
| 418203 | X54942 | Hs. 83758 | CDC28 protein kinase 2 | 4.57E−03 | 2.45 |
| 423983 | AA333261 | | gb: EST37476 Embryo, 8 week 1 Homo sapien | 1.10E−02 | 2.45 |
| 434128 | W93170 | Hs. 284164 | protein x 0004 | 1.62E−04 | 2.45 |
| 438141 | AW946871 | | gb: RC2-ET0022-080500-012-d02 ET0022 Homo | 3.34E−03 | 2.45 |
| 449239 | T24653 | Hs. 23360 | likely ortholog of yeast ARV1 | 3.06E−06 | 2.45 |
| 422241 | Y00062 | Hs. 170121 | protein tyrosine phosphatase, receptor t | 1.49E−05 | 2.45 |
| 414462 | BE622743 | Hs. 301064 | arfaptin 1 | 5.44E−03 | 2.44 |
| 423706 | U95218 | Hs. 131924 | G protein-coupled receptor 65 | 2.55E−03 | 2.44 |
| 431661 | AB037830 | Hs. 267150 | KIAA1409 protein | 1.71E−04 | 2.44 |
| 413856 | D13639 | Hs. 75586 | cyclin D2 | 4.57E−03 | 2.44 |
| 422684 | BE561617 | Hs. 119192 | H2A histone family, member Z | 6.72E−03 | 2.44 |
| 420623 | BE245485 | Hs. 99437 | Homo sapiens mRNA; cDNA DKFZp586G1924 (f | 6.45E−03 | 2.43 |
| 417377 | NM_016603 | Hs. 82035 | potential nuclear protein C5ORF5; GAP-li | 1.79E−05 | 2.43 |
| 409884 | AI904455 | Hs. 142684 | hypothetical protein DKFZp667O116 | 1.59E−03 | 2.43 |
| 452748 | AB011128 | Hs. 30512 | Homo sapiens mRNA for KIAA0556 protein, | 1.31E−03 | 2.43 |
| 434500 | AF143877 | Hs. 215047 | Homo sapiens clone IMAGE: 113431 mRNA seq | 3.49E−02 | 2.43 |
| 436021 | R26877 | Hs. 24128 | ESTs | 3.25E−02 | 2.42 |
| 442149 | AB014550 | Hs. 8118 | KIAA0650 protein | 3.50E−03 | 2.42 |
| 408411 | C15118 | Hs. 322482 | hypothetical protein DKFZp566J2046 | 9.48E−06 | 2.42 |
| 407656 | AW747986 | Hs. 37443 | Homo sapiens mRNA; cDNA DKFZp434B2119 (f | 4.08E−04 | 2.42 |
| 430587 | AK000341 | Hs. 246107 | elongation of very long chain fatty acid | 4.52E−05 | 2.41 |
| 412019 | AA485890 | Hs. 69330 | Homo sapiens cDNA FLJ13835 fis, clone TH | 1.81E−04 | 2.41 |
| 457650 | AA649162 | Hs. 236456 | ESTs | 4.25E−05 | 2.41 |
| 458971 | AL119206 | Hs. 101874 | ESTs, Weakly similar to ALU1_HUMAN ALU S | 2.28E−02 | 2.41 |
| 433902 | AW292820 | Hs. 144906 | ESTs | 5.91E−03 | 2.41 |
| 439645 | BE091801 | Hs. 27167 | ESTs, Weakly similar to I38022 hypotheti | 1.77E−06 | 2.41 |
| 430007 | NM_014892 | Hs. 227602 | KIAA1116 protein | 2.32E−03 | 2.41 |
| 427547 | BE047653 | Hs. 119183 | ESTs, Weakly similar to ZN91_HUMAN ZINC | 3.49E−03 | 2.41 |
| 448749 | AW859679 | Hs. 21902 | Homo sapiens clone 25237 mRNA sequence | 1.84E−03 | 2.40 |
| 444913 | AI362726 | Hs. 193656 | Homo sapiens mRNA for KIAA1658 protein, | 5.39E−05 | 2.40 |
| 428466 | AF151063 | Hs. 184456 | hypothetical protein | 4.00E−03 | 2.40 |
| 408636 | BE294925 | Hs. 46680 | CGI-12 protein | 3.06E−03 | 2.40 |
| 416517 | AA775987 | Hs. 79357 | proteasome (prosome, macropain) 26S subu | 1.06E−02 | 2.40 |
| 438041 | AI394551 | | ESTs | 4.56E−02 | 2.40 |
| 444088 | AW297946 | Hs. 138208 | ESTs | 2.54E−02 | 2.40 |
| 449118 | R67477 | Hs. 23103 | Bet1 (S. cerevisiae) homolog | 2.93E−02 | 2.40 |
| 452253 | AA928891 | Hs. 28608 | Homo sapiens cDNA: FLJ22115 fis. clone H | 1.18E−03 | 2.40 |
| 435872 | AA701357 | Hs. 192759 | ESTs | 1.59E−03 | 2.40 |
| 409132 | AJ224538 | Hs. 50732 | protein kinase, AMP-activated, beta 2 no | 1.25E−06 | 2.40 |
| 414256 | AW410035 | Hs. 75862 | MAD (mothers against decapentaplegic, Dr | 2.93E−02 | 2.39 |
| 459680 | H96982 | Hs. 42321 | ESTs | 2.14E−04 | 2.39 |
| 427657 | AV652249 | Hs. 180107 | polymerase (DNA directed), beta | 7.07E−07 | 2.39 |
| 446570 | AV659177 | Hs. 127160 | ESTs | 2.31E−05 | 2.38 |
| 415668 | AW957684 | Hs. 306814 | hypothetical protein FLJ21889 | 4.57E−02 | 2.38 |
| 428985 | AL134193 | Hs. 194709 | paraneoplastic antigen MA1 | 4.00E−03 | 2.38 |
| 438030 | X98427 | Hs. 122634 | ESTs | 2.33E−03 | 2.38 |
| 457605 | AV657778 | Hs. 3314 | selenoprotein P, plasma, 1 | 2.32E−03 | 2.38 |
| 422043 | AL133649 | Hs. 110953 | retinoic acid induced 1 | 1.31E−03 | 2.38 |
| 425375 | AA631977 | Hs. 155995 | KIAA0643 protein | 2.44E−05 | 2.38 |
| 427020 | AA397546 | Hs. 119151 | ESTs | 1.01E−02 | 2.38 |
| 440341 | AW664012 | Hs. 132333 | ESTs | 1.93E−03 | 2.38 |
| 424321 | W74048 | Hs. 1765 | lymphocyte-specific protein tyrosine kin | 2.38E−04 | 2.37 |
| 416611 | AA568308 | | ESTs, Weakly similar to ALU6_HUMAN ALU S | 2.67E−03 | 2.37 |
| 416065 | BE267931 | Hs. 78996 | proliferating cell nuclear antigen | 6.21E−04 | 2.37 |
| 419424 | BE041820 | Hs. 38516 | Homo sapiens, clone MGC: 15887, mRNA, com | 4.30E−04 | 2.36 |
| 452207 | NM_014517 | Hs. 28423 | upstream binding protein 1 (LBP-1a) | 5.71E−05 | 2.36 |
| 414496 | W73853 | | ESTs | 1.92E−03 | 2.36 |
| 451107 | AA235108 | Hs. 17639 | Homo sapiens ubiquitin protein ligase (U | 7.59E−04 | 2.35 |
| 452685 | AI634651 | Hs. 30250 | v-maf musculoaponeurotic fibrosarcoma (a | 6.41E−06 | 2.35 |
| 411196 | W31212 | Hs. 69192 | vacuolar protein sorting 29 (yeast homol | 2.45E−02 | 2.35 |
| 427647 | W19744 | Hs. 180059 | Homo sapiens cDNA FLJ20653 fis, clone KA | 1.62E−04 | 2.35 |
| 422607 | Z45471 | Hs. 118684 | stromal cell-derived factor 2 | 4.30E−04 | 2.35 |
| 427469 | AA403084 | Hs. 269347 | ESTs, Weakly similar to 2109260A B cell | 9.74E−03 | 2.35 |
| 429065 | AI753247 | Hs. 29643 | Homo sapiens cDNA FLJ13103 fis, clone NT | 1.06E−02 | 2.35 |
| 439103 | AF085959 | Hs. 38705 | ESTs | 2.43E−03 | 2.35 |
| 427094 | AB025254 | Hs. 283761 | tudor repeat associated with PCTAIRE 2 | 7.02E−07 | 2.35 |
| 451789 | AW291532 | Hs. 211297 | ESTs | 3.20E−03 | 2.35 |
| 414436 | U50078 | Hs. 76127 | hect (homologous to the E6-AP (UBE3A) ca | 5.43E−03 | 2.35 |
| 446487 | AA195526 | Hs. 44625 | Rad50-interacting protein 1 | 2.79E−03 | 2.34 |
| 411580 | AL080088 | Hs. 70877 | DKFZP564K2062 protein | 1.80E−04 | 2.34 |
| 413880 | AI660842 | Hs. 110915 | interleukin 22 receptor | 5.87E−04 | 2.33 |
| 431899 | AA521381 | Hs. 187726 | ESTs | 1.06E−02 | 2.33 |
| 450607 | AL050373 | Hs. 25213 | hypothetical protein | 2.30E−05 | 2.33 |
| 440726 | AL050333 | Hs. 306425 | DKFZP564B116 protein | 4.14E−02 | 2.33 |
| 417089 | H52280 | Hs. 18612 | Homo sapiens cDNA: FLJ21909 fis, clone H | 1.17E−06 | 2.33 |
| 450919 | AA011616 | Hs. 269877 | ESTs | 6.72E−03 | 2.33 |
| 414312 | AA155694 | Hs. 191060 | ESTs | 5.88E−04 | 2.32 |
| 421025 | AW958975 | Hs. 29397 | Homo sapiens cDNA FLJ13226 fis, clone OV | 2.26E−04 | 2.32 |
| 439556 | AI623752 | Hs. 163603 | ESTs | 2.36E−02 | 2.32 |
| 424528 | AW073971 | Hs. 238954 | ESTs, Weakly similar to KIAA1204 protein | 1.25E−03 | 2.32 |
| 423365 | AA324992 | Hs. 257168 | ESTs | 3.20E−03 | 2.32 |
| 403920 | | | suppressor of potassium transport defect | 4.14E−02 | 2.32 |
| 448280 | AW014215 | Hs. 357 | zinc finger protein 134 (clone pHZ-15) | 2.54E−03 | 2.32 |
| 407687 | AK002011 | Hs. 37558 | hypothetical protein FLJ11149 | 3.99E−03 | 2.31 |
| 409259 | AW608930 | Hs. 52184 | hypothetical protein FLJ20618 | 2.16E−06 | 2.30 |
| 407804 | AF228603 | Hs. 39957 | pleckstrin 2 (mouse) homolog | 5.21E−03 | 2.30 |
| 423968 | AF098277 | Hs. 136529 | solute carrier family 23 (nucleobase tra | 1.31E−05 | 2.30 |
| 414525 | C14904 | Hs. 45184 | Homo sapiens cDNA FLJ12284 fis, clone MA | 1.44E−03 | 2.30 |
| 420164 | AW339037 | Hs. 24908 | ESTs | 2.63E−02 | 2.30 |
| 422900 | AA641201 | Hs. 222051 | ESTs | 1.08E−03 | 2.30 |
| 423799 | AW026300 | Hs. 132906 | 19A24 protein | 3.35E−03 | 2.30 |
| 440390 | AW207385 | Hs. 36475 | KIAA0493 protein | 1.92E−03 | 2.30 |
| 456351 | AW971067 | Hs. 293056 | ESTs, Weakly similar to 138022 hypotheti | 3.50E−03 | 2.30 |
| 431341 | AA307211 | Hs. 251531 | proteasome (prosome, macropain) subunit. | 1.22E−04 | 2.30 |
| 413029 | AL119399 | Hs. 293850 | ESTs | 5.05E−05 | 2.30 |
| 453315 | BE544203 | Hs. 24831 | ESTs | 8.62E−03 | 2.30 |
| 433160 | AW207002 | Hs. 134342 | TASP for testis-specific adriamycin sens | 1.96E−02 | 2.30 |
| 420137 | AA306478 | Hs. 95327 | CD3D antigen, delta polypeptide (TiT3 co | 6.39E−06 | 2.30 |
| 436252 | AI539519 | Hs. 120969 | Homo sapiens cDNA FLJ11562 fis, clone HE | 1.03E-−03 | 2.30 |
| 437143 | AW204056 | Hs. 8917 | ESTs | 6.58E−07 | 2.30 |
| 440495 | AA887212 | Hs. 14161 | hypothetical protein DKFZp43411930 | 1.75E−03 | 2.30 |
| 419203 | AA488719 | Hs. 190151 | ESTs | 8.98E−03 | 2.30 |
| 410017 | AW952426 | Hs. 109438 | Homo sapiens clone 24775 mRNA sequence | 2.02E−04 | 2.29 |
| 415173 | AW501735 | Hs. 180059 | ESTs | 4.28E−02 | 2.29 |
| 419200 | AW966405 | | prefoldin 5 | 7.61E−07 | 2.29 |
| 437109 | AW006781 | Hs. 5457 | hypothetical protein FLJ10738 | 3.12E−04 | 2.29 |
| 438475 | W03856 | Hs. 13188 | ESTs, Highly similar to Gene product wit | 5.26E−06 | 2.28 |
| 417206 | AA291183 | Hs. 81648 | hypothetical protein FLJ11021 similar to | 2.66E−03 | 2.27 |
| 448410 | AK000227 | Hs. 21126 | hypothetical protein FLJ20220 | 7.37E−02 | 2.27 |
| 448959 | AI610343 | Hs. 293267 | ESTs | 1.59E−03 | 2.27 |
| 440528 | BE313555 | Hs. 7252 | KIAA1224 protein | 1.01E−02 | 2.27 |
| 421620 | AA446183 | Hs. 91885 | ESTs, Weakly similar to 155214 salivary | 6.17E−03 | 2.27 |
| 451589 | M424791 | Hs. 5734 | meningioma expressed antigen 5 (hyaluron | 4.56E−03 | 2.27 |
| 421931 | NM_000814 | Hs. 1440 | gamma-aminobutyric acid (GABA) A recepto | 1.62E−04 | 2.27 |
| 431831 | AW023204 | Hs. 302743 | ESTs | 8.40E−04 | 2.27 |
| 428583 | AA430589 | Hs. 75410 | heat shock 70 kD protein 5 (glucose-regul | 5.91E−03 | 2.26 |
| 452248 | AA093668 | Hs. 28578 | muscleblind (Drosophila)-like | 9.71E−05 | 2.26 |
| 446045 | AV656268 | Hs. 209153 | angiopoietin-like 3 | 1.13E−03 | 2.26 |
| 421297 | AB037360 | | KIAA1255 protein | 3.13E−05 | 2.26 |
| 440945 | AW505345 | Hs. 7540 | f-box and leucine-rich repeat protein 3A | 9.10E−02 | 2.26 |
| 452852 | AK001972 | Hs. 30822 | hypothetical protein FLJ11110 | 1.31E−05 | 2.26 |
| 403496 | | | C3000956*: gi\|7710129\|ref\|NP_006141.2\|LI | 1.19E−03 | 2.26 |
| 439046 | M947354 | | gb: od86e11.s1 NCI_CGAP_Ov2 Homo sapiens | 4.78E−04 | 2.25 |
| 427268 | X78520 | Hs. 174139 | chloride channel 3 | 6.19E−04 | 2.25 |
| 405122 | | | NM_006798*: Homo sapiens UDP glycosyltran | 1.59E−03 | 2.25 |
| 403320 | | | fms-related tyrosine kinase 3 | 1.89E−02 | 2.25 |
| 416754 | H07145 | Hs. 6799 | ESTs, Weakly similar to T12483 hypotheti | 1.52E−03 | 2.25 |
| 418046 | W49670 | Hs. 56044 | ESTs | 2.04E−02 | 2.25 |
| 418383 | AA218986 | Hs. 118854 | ESTs | 1.96E−02 | 2.25 |
| 429025 | AI399910 | Hs. 266782 | KIAA1826 protein | 1.06E−02 | 2.25 |
| 435756 | AI418466 | Hs. 33665 | ESTs | 2.01E−03 | 2.25 |
| 440638 | AI376551 | | gb: te64e10.x1 Soares_NFL_T_GBC.S1 Homo s | 1.39E−02 | 2.25 |
| 447225 | R62676 | Hs. 17820 | Rho-associated, coiled-coil containing p | 2.81E−04 | 2.25 |
| 434747 | AA837085 | | ESTs | 1.25E−03 | 2.24 |
| 453041 | AI680737 | Hs. 289068 | Homo sapiens cDNA FLJ11918 fis, clone HE | 2.45E−02 | 2.24 |
| 445044 | AL137728 | Hs. 12258 | Homo sapiens mRNA; cDNA DKFZp434B0920 (f | 5.56E−02 | 2.24 |
| 448939 | BE267795 | Hs. 22595 | hypothetical protein FLJ10637 | 1.01E−02 | 2.24 |
| 450196 | AW956868 | Hs. 24608 | DKFZP564D177 protein | 4.52E−05 | 2.23 |
| 453929 | AW190054 | | gb: xl11d05.x1 NCI_CGAP_Ut4 Homo sapiens | 2.04E−02 | 2.23 |
| 455959 | AA131782 | Hs. 182314 | ESTs | 1.10E−02 | 2.23 |
| 446594 | AI311917 | Hs. 16292 | ESTs | 9.93E−02 | 2.23 |
| 445893 | AI610702 | Hs. 202613 | ESTs, Weakly similar to TRHY_HUMAN TRICH | 1.09E−04 | 2.23 |
| 432476 | T94344 | Hs. 326263 | ESTs | 1.56E−02 | 2.23 |
| 448888 | AW196663 | Hs. 200242 | caspase recruitment domain protein 6 | 7.58E−04 | 2.23 |
| 405270 | | | NM_018850*: Homo sapiens ATP-binding cass | 1.02E−02 | 2.22 |
| 442806 | AW294522 | Hs. 149991 | ESTs | 2.45E−02 | 2.22 |
| 450314 | AA574309 | Hs. 283402 | TCR eta | 4.77E−03 | 2.22 |
| 452144 | AA032197 | Hs. 102558 | Homo sapiens, clone MGC: 5352, mRNA, comp | 2.39E−04 | 2.22 |
| 452436 | BE077546 | Hs. 31447 | ESTs, Moderately similar to A46010 X-lin | 3.66E−03 | 2.22 |
| 455834 | BE145364 | | gb: IL0-HT0198-151099-125-e05 HT0198 Homo | 1.14E−02 | 2.22 |
| 436643 | AA757626 | Hs. 10941 | ESTs, Weakly similar to IPP1_HUMAN PROTE | 5.03E−04 | 2.22 |
| 448558 | AW340579 | Hs. 171165 | ESTs | 6.51E−04 | 2.22 |
| 414091 | T83742 | Hs. 334616 | gb: yd67g02.s1 Soares fetal liver spleen | 2.80E−04 | 2.21 |
| 421958 | AA357185 | Hs. 109918 | ras homolog gene family, member H | 5.58E−04 | 2.21 |
| 433648 | AA603388 | Hs. 44131 | KIAA0974 protein | 1.75E−03 | 2.21 |
| 428004 | AA449563 | Hs. 151393 | glutamate-cysteine ligase, catalytic sub | 4.72E−02 | 2.21 |
| 446229 | AI744964 | Hs. 14449 | KIAA1609 protein | 5.58E−04 | 2.21 |
| 421921 | H83363 | | translocase of inner mitochondrial membr | 8.81E−04 | 2.20 |
| 408866 | AW292096 | Hs. 255036 | ESTs | 1.14E−02 | 2.20 |
| 400120 | | | Eos Control | 1.82E−02 | 2.20 |
| 415000 | AW025529 | Hs. 239812 | Homo sapiens serologically defined breas | 1.61E−04 | 2.20 |
| 408990 | AL022395 | Hs. 49526 | f-box and leucine-rich repeat protein 4 | 3.19E−03 | 2.20 |
| 422371 | NM_001882 | Hs. 115617 | corticotropin releasing hormone-binding | 1.15E−04 | 2.20 |
| 437580 | AA761075 | | ESTs | 2.04E−02 | 2.20 |
| 453927 | AA082465 | Hs. 125031 | choline/ethanolaminephosphotransferase | 4.60E−06 | 2.20 |
| 405688 | | | NM_018850*: Homo sapiens ATP-binding cass | 2.52E−04 | 2.20 |
| 427130 | AB029020 | Hs. 173694 | KIAA1097 protein | 6.45E−05 | 2.20 |
| 414718 | H95348 | Hs. 107987 | ESTs | 1.96E−02 | 2.20 |
| 409549 | AB029015 | Hs. 54886 | phospholipase C, epsilon 2 | 1.51E−03 | 2.20 |
| 424904 | AI221739 | Hs. 96899 | ESTs | 8.95E−03 | 2.20 |
| 449103 | T24968 | Hs. 23038 | HSPC071 protein | 3.87E−04 | 2.20 |
| 413326 | H88621 | Hs. 19762 | ESTs, Weakly similar to KIAA1140 protein | 2.63E−02 | 2.19 |
| 426011 | AW996096 | Hs. 58924 | ESTs, Weakly similar to JC5594 jerky gen | 1.67E−03 | 2.19 |
| 443352 | H70284 | Hs. 160152 | ESTs, Weakly similar to FPHU alpha-fetop | 9.75E−03 | 2.19 |
| 427156 | BE621719 | Hs. 173802 | KIAA0603 gene product | 4.14E−02 | 2.19 |
| 418980 | T77130 | Hs. 268606 | ESTs | 3.60E−02 | 2.19 |
| 425745 | U44060 | Hs. 159437 | Homo sapiens cDNA: FLJ21800 fis, clone H | 2.55E−03 | 2.19 |
| 433162 | AI025842 | | ESTs | 2.96E−04 | 2.19 |
| 412240 | H72176 | | hypothetical protein FLJ13159 | 3.35E−03 | 2.19 |
| 440113 | AI916532 | Hs. 188272 | ESTs | 4.35E−03 | 2.19 |
| 430526 | AF181862 | Hs. 242407 | G protein-coupled receptor, family C, gr | 2.81E−04 | 2.18 |
| 447881 | BE620886 | | GCN1 (general control of amino-acid synt | 1.51E−02 | 2.18 |
| 407151 | H25836 | Hs. 301527 | ESTs, Moderately similar to unknown [H.s | 4.59E−07 | 2.18 |
| 411590 | T96183 | | gb: ye09f07.s1 Stratagene lung (937210) H | 3.04E−02 | 2.18 |
| 413823 | AI341417 | Hs. 29406 | ESTs | 6.00E−06 | 2.18 |
| 401627 | | | Target Exon | 8.62E−03 | 2.18 |
| 414588 | AA302905 | | gb: EST10607 Adipose tissue, white I Homo | 9.77E−04 | 2.18 |
| 425508 | AA991551 | Hs. 97013 | Homo sapiens, Similar to RIKEN cDNA 2310 | 8.14E−05 | 2.18 |
| 434260 | AF121856 | Hs. 284291 | sorting nexin 6 | 4.52E−05 | 2.18 |
| 407904 | W44735 | Hs. 9286 | Homo sapiens cDNA: FLJ21278 fis, clone C | 8.83E−04 | 2.17 |
| 421077 | AK000061 | Hs. 101590 | hypothetical protein | 2.54E−02 | 2.17 |
| 427120 | R42099 | Hs. 21965 | ESTs | 1.37E−03 | 2.17 |
| 442129 | N36918 | Hs. 20142 | PNAS-127 protein | 4.37E−03 | 2.17 |
| 444291 | AI598022 | Hs. 193989 | TAR DNA binding protein | 3.99E−03 | 2.17 |
| 418416 | U11700 | Hs. 84999 | ATPase, Cu transporting, beta polypeptid | 1.79E−05 | 2.17 |
| 403576 | | | C3000124: gi\|12737057\|ref\|XP_012129.1\|si | 5.20E−03 | 2.17 |
| 446126 | AW085909 | | pleckstrin homology domain interacting p | 6.72E−03 | 2.17 |
| 424699 | AW206227 | Hs. 287727 | hypothetical protein FLJ23132 | 8.99E−03 | 2.17 |
| 437223 | C15105 | Hs. 330716 | Homo sapiens cDNA FLJ14368 fis, clone HE | 2.02E−03 | 2.17 |
| 432540 | AI821517 | Hs. 105866 | ESTs | 2.35E−02 | 2.17 |
| 425266 | J00077 | Hs. 155421 | alpha-fetoprotein | 1.14E−02 | 2.17 |
| 438619 | AB032773 | | TU12B1-TY protein | 3.86E−02 | 2.17 |
| 442053 | R35343 | Hs. 24968 | Human DNA sequence from clone RP1-233G16 | 9.74E−03 | 2.16 |
| 430308 | BE540865 | Hs. 238990 | cyclin-dependent kinase inhibitor 1B (p2 | 4.25E−05 | 2.16 |
| 434608 | AA805443 | Hs. 179909 | hypothetical protein FLJ22995 | 4.37E−03 | 2.16 |
| 408527 | AL135018 | Hs. 33074 | Homo sapiens, clone IMAGE: 3606519, mRNA, | 4.28E−02 | 2.16 |
| 410612 | AW502698 | Hs. 42400 | ESTs | 9.16E−05 | 2.16 |
| 410800 | BE280421 | Hs. 94499 | ESTs | 1.25E−03 | 2.15 |
| 456073 | AA587775 | Hs. 66295 | multi-PDZ-domain-containing protein | 3.76E−05 | 2.15 |
| 411468 | AW857470 | | gb: CM3-CT0310-180200-098-g03 CT0310 Homo | 1.19E−03 | 2.15 |
| 411979 | X85134 | Hs. 72984 | retinoblastoma-binding protein 5 | 5.21E−03 | 2.15 |
| 419165 | AW860767 | Hs. 118879 | ESTs | 1.14E−02 | 2.15 |
| 419479 | AI288348 | Hs. 23450 | mitochondrial ribosomal protein S25 | 7.60E−02 | 2.15 |
| 432709 | H17238 | | gb: ym42f03.r1 Soares infant brain 1NIB H | 3.65E−03 | 2.15 |
| 448705 | H05072 | Hs. 124984 | ESTs, Moderately similar to ALU7_HUMAN A | 6.51E−02 | 2.15 |
| 440266 | AA088809 | Hs. 19525 | hypothetical protein FLJ22794 | 1.29E−04 | 2.15 |
| 449881 | Z28444 | Hs. 24119 | Homo sapiens mRNA; cDNA DKFZp586G2222 (f | 5.68E−03 | 2.15 |
| 457500 | NM_002759 | Hs. 274382 | protein kinase, interferon-inducible dou | 1.10E−02 | 2.15 |
| 417713 | D42047 | Hs. 82432 | KIAA0089 protein | 3.66E−03 | 2.14 |
| 416269 | AA177138 | Hs. 161671 | ESTs | 8.29E−03 | 2.14 |
| 437370 | AL359567 | Hs. 161962 | Homo sapiens mRNA; cDNA DKFZp547D023 (fr | 2.55E−03 | 2.14 |
| 413305 | NM_000426 | Hs. 323511 | Homo sapiens cDNA: FLJ23176 fis, clone L | 6.93E−02 | 2.14 |
| 413012 | D83777 | Hs. 75137 | KIAA0193 gene product | 3.50E−03 | 2.14 |
| 431874 | AW610031 | Hs. 323914 | translocase of inner mitochondrial membr | 9.73E−03 | 2.14 |
| 421166 | AA305407 | Hs. 102308 | potassium inwardly-rectifying channel, s | 3.06E−03 | 2.13 |
| 414883 | AA926960 | | CDC28 protein kinase 1 | 6.41E−06 | 2.13 |
| 426685 | R20212 | Hs. 28454 | ESTs | 7.31E−03 | 2.13 |
| 407874 | AI766311 | Hs. 289047 | Homo sapiens cDNA FLJ14059 fis, clone HE | 4.92E−06 | 2.13 |
| 423703 | NM_014913 | Hs. 131915 | KIAA0863 protein | 8.83E−04 | 2.13 |
| 443804 | AL135352 | Hs. 255883 | ESTs, Weakly similar to I38022 hypotheti | 1.52E−03 | 2.13 |
| 436100 | AA704806 | Hs. 143842 | ESTs, Weakly similar to 2004399A chromos | 8.32E−02 | 2.13 |
| 444931 | AV652066 | Hs. 75113 | general transcription factor IIIA | 4.01E−03 | 2.13 |
| 403742 | | | Target Exon | 8.00E−04 | 2.13 |
| 407183 | AA358015 | | gb: EST66864 Fetal lung III Homo sapiens | 3.47E−04 | 2.13 |
| 430048 | T65054 | Hs. 73605 | ESTs | 1.29E−02 | 2.13 |
| 445800 | AA126419 | Hs. 32944 | inositol polyphosphate-4-phosphatase, ty | 2.54E−02 | 2.13 |
| 438829 | AA826926 | Hs. 204214 | ESTs, Weakly similar to 138022 hypotheti | 1.14E−02 | 2.12 |
| 443189 | AB023179 | Hs. 9059 | KIAA0962 protein | 3.49E−03 | 2.12 |
| 421633 | AF121860 | Hs. 106260 | sorting nexin 10 | 6.51E−02 | 2.12 |
| 408475 | AA315514 | Hs. 47986 | hypothetical protein MGC10940 | 5.93E−02 | 2.12 |
| 436237 | R11528 | Hs. 271968 | ESTs | 2.39E−04 | 2.12 |
| 409509 | AL036923 | Hs. 322710 | ESTs | 3.74E−02 | 2.11 |
| 408358 | D20044 | Hs. 12929 | hypothetical protein FLJ20721 | 2.91E−03 | 2.11 |
| 417867 | AW952547 | Hs. 194603 | ESTs, Moderately similar to I38022 hypot | 1.68E−02 | 2.10 |
| 417691 | AU076610 | Hs. 82399 | low density lipoprotein receptor defect | 1.19E−03 | 2.10 |
| 439161 | Y15164 | Hs. 6483 | oral-facial-digital syndrome 1 | 1.84E−03 | 2.10 |
| 441024 | AW081530 | Hs. 268231 | ESTs | 7.28E−03 | 2.10 |
| 445817 | NM_003642 | Hs. 13340 | histone acetyltransferase 1 | 2.12E−03 | 2.10 |
| 446783 | AW138343 | Hs. 141867 | ESTs | 9.75E−03 | 2.10 |
| 449901 | AI674072 | | gb: wd15h01.x1 Soares_NFL_T_{—} | 2.95E−05 | 2.10 |
| | | | GBC_S1 Homo s | | |
| 456373 | BE247706 | Hs. 89751 | membrane-spanning 4-domains, subfamily A | 9.78E−04 | 2.10 |
| 451582 | AI963026 | Hs. 289958 | ESTs, Weakly similar to putative p150 [H | 1.24E−02 | 2.10 |
| 432106 | N58323 | Hs. 269098 | ESTs, Weakly similar to RETROVIRUS-RELAT | 2.43E−03 | 2.10 |
| 426295 | AW367283 | | zinc finger protein 6 (CMPX1) | 1.30E−04 | 2.09 |
| 426272 | AW450671 | Hs. 189284 | ESTs | 4.52E−05 | 2.09 |
| 444647 | H14718 | Hs. 11506 | Human clone 23589 mRNA sequence | 6.93E−02 | 2.09 |
| 458476 | AA336878 | Hs. 9842 | Human DNA sequence from clone RP4-788L20 | 2.92E−03 | 2.09 |
| 428220 | BE183533 | Hs. 347128 | Human DNA sequence from clone 34B21 on c | 6.84E−06 | 2.09 |
| 446103 | U90918 | Hs. 13804 | hypothetical protein dJ462O23.2 | 4.76E−03 | 2.09 |
| 431742 | NM_016652 | Hs. 268281 | crooked neck protein (cm) | 1.10E−02 | 2.09 |
| 412760 | AW379030 | Hs. 41324 | ESTs | 3.99E−05 | 2.08 |
| 450203 | AF097994 | Hs. 301528 | L-kynurenine/alpha-aminoadipate aminotra | 8.57E−02 | 2.08 |
| 405086 | | | NM_006662*: Homo sapiens Snf2-related CBP | 1.82E−02 | 2.08 |
| 429567 | R35606 | Hs. 326800 | Human EST clone 53125 mariner transposon | 2.36E−02 | 2.08 |
| 418459 | R85436 | Hs. 268814 | ESTs | 2.21E−03 | 2.07 |
| 424737 | BE301883 | Hs. 152707 | glioblastoma amplified sequence | 4.77E−04 | 2.07 |
| 454038 | X06374 | Hs. 37040 | platelet-derived growth factor alpha pol | 1.75E−02 | 2.07 |
| 427468 | AB036829 | Hs. 178347 | SKIP for skeletal muscle and kidney enri | 1.25E−03 | 2.07 |
| 412748 | BE083158 | Hs. 10862 | Homo sapiens cDNA: FLJ23313 fis, clone H | 4.56E−03 | 2.07 |
| 455705 | AW161061 | | ESTs, Weakly similar to zinc finger prot | 2.36E−07 | 2.07 |
| 421483 | NM_003388 | Hs. 104717 | hypothetical protein MGC11333 | 1.19E−02 | 2.06 |
| 433713 | AW976511 | Hs. 112592 | ESTs | 2.83E−02 | 2.06 |
| 457171 | AA433896 | Hs. 201634 | ESTs | 1.29E−02 | 2.06 |
| 433201 | AB040896 | Hs. 21104 | KIAA1463 protein | 7.30E−03 | 2.06 |
| 432841 | M93425 | Hs. 62 | protein tyrosine phosphatase, non-recept | 2.83E−02 | 2.06 |
| 437469 | AW753112 | Hs. 15514 | hypothetical protein MGC3260 | 2.92E−03 | 2.06 |
| 402929 | | | ENSP00000243914*: DJ579F20.2 (similar to | 4.00E−03 | 2.06 |
| 408246 | N55669 | Hs. 333823 | mitochondrial ribosomal protein L13 | 4.50E−05 | 2.06 |
| 410582 | AW867197 | Hs. 337561 | hypothetical protein FLJ21616 | 1.15E−05 | 2.05 |
| 411495 | AP000693 | Hs. 70359 | KIAA0136 protein | 5.38E−02 | 2.05 |
| 412059 | AA317962 | Hs. 249721 | ESTs, Moderately similar to PC4259 ferri | 6.72E−02 | 2.05 |
| 414548 | AW937036 | Hs. 183506 | hypothetical protein FLJ14213 | 3.29E−04 | 2.05 |
| 433735 | AA608955 | Hs. 109653 | ESTs | 3.14E−02 | 2.05 |
| 441872 | BE567100 | Hs. 154938 | hypothetical protein MDS025 | 2.04E−05 | 2.05 |
| 448920 | AW408009 | Hs. 22580 | alkylglycerone phosphate synthase | 5.92E−03 | 2.05 |
| 448980 | AL137527 | Hs. 289038 | hypothetical protein MGC4126 | 5.43E−03 | 2.05 |
| 453078 | AF053551 | Hs. 31584 | metaxin 2 | 7.28E−03 | 2.05 |
| 453574 | AI767947 | Hs. 50841 | ESTs | 2.63E−02 | 2.05 |
| 410664 | NM_006033 | Hs. 65370 | lipase, endothelial | 7.31E−03 | 2.05 |
| 436856 | AI469355 | Hs. 127310 | ESTs | 2.87E−06 | 2.05 |
| 408483 | AA464836 | Hs. 291079 | ESTs, Weakly similar to T27173 hypotheti | 4.24E−05 | 2.05 |
| 444775 | AI040384 | Hs. 19102 | ESTs, Weakly similar to organic anion tr | 6.08E−05 | 2.04 |
| 423996 | AF205071 | Hs. 137425 | solute carrier family 21 (organic anion | 7.84E−02 | 2.04 |
| 451334 | AI122691 | Hs. 13268 | ESTs | 9.76E−04 | 2.04 |
| 418504 | BE159718 | Hs. 85335 | Homo sapiens mRNA; cDNA DKFZp564D1462 (f | 8.16E−05 | 2.04 |
| 457830 | BE147896 | Hs. 14662 | ESTs | 7.61E−03 | 2.04 |
| 408194 | AA601038 | Hs. 191797 | ESTs, Weakly similar to S65657 alpha-1C- | 4.72E−02 | 2.04 |
| 428706 | AA432030 | Hs. 265827 | interferon, alpha-inducible protein (clo | 2.68E−06 | 2.04 |
| 435841 | R28522 | Hs. 186937 | ESTs | 8.82E−04 | 2.03 |
| 429586 | T73510 | Hs. 209153 | angiopoietin-like 3 | 1.29E−04 | 2.03 |
| 453173 | AB007902 | Hs. 32168 | KIAA0442 protein | 5.73E−05 | 2.03 |
| 415662 | AW972481 | Hs. 170610 | ESTs, Highly similar to G01887 MEK kinas | 2.02E−04 | 2.03 |
| 407879 | AA045464 | Hs. 6557 | zinc finger protein 161 | 1.89E−02 | 2.02 |
| 417303 | NM_001698 | Hs. 81886 | AU RNA-binding protein/enoyl-Coenzyme A | 2.02E−03 | 2.02 |
| 429857 | AF089897 | Hs. 294030 | topoisomerase-related function protein 4 | 8.03E−05 | 2.02 |
| 432886 | BE159028 | Hs. 279704 | chromatin accessibility complex 1 | 7.38E−02 | 2.02 |
| 432960 | AW150945 | Hs. 8739 | ESTs | 1.51E−02 | 2.02 |
| 436797 | AA731491 | Hs. 334477 | hypothetical protein MGC14879 | 3.73E−05 | 2.02 |
| 439564 | W77911 | Hs. 110006 | ESTs | 5.56E−02 | 2.02 |
| 449845 | AW971183 | Hs. 6019 | DnaJ (Hsp40) homolog, subfamily C, membe | 6.20E−04 | 2.02 |
| 419426 | AI214690 | Hs. 346257 | aldo-keto reductase family 1, member B1 | 3.19E−03 | 2.02 |
| 424571 | BE379766 | | polymerase (RNA) II (DNA directed) polyp | 1.24E−06 | 2.02 |
| 441965 | AA972712 | Hs. 269737 | ESTs | 4.77E−03 | 2.02 |
| 421456 | AW579842 | Hs. 104557 | hypothetical protein FLJ10697 | 1.96E−02 | 2.02 |
| 408395 | BE072425 | Hs. 44579 | hypothetical protein FLJ20199 | 5.59E−04 | 2.02 |
| 427561 | AI123333 | Hs. 134191 | ESTs | 2.67E−06 | 2.02 |
| 411678 | AI907114 | Hs. 71465 | squalene epoxidase | 3.03E−02 | 2.02 |
| 448873 | NM_003677 | Hs. 22393 | density-regulated protein | 1.24E−02 | 2.02 |
| 420747 | BE294407 | Hs. 99910 | phosphofructokinase, platelet | 9.75E−03 | 2.02 |
| 430213 | AW993446 | Hs. 235445 | hypothetical protein FLJ21313 | 4.42E−02 | 2.02 |
| 417971 | Y08991 | Hs. 83050 | phosphoinositide-3-kinase, regulatory su | 1.19E−02 | 2.01 |
| 424247 | X14008 | Hs. 234734 | lysozyme (renal amyloidosis) | 3.06E−03 | 2.01 |
| 404240 | | | NM_018950*: Homo sapiens major histocompat | 2.45E−05 | 2.01 |
| 400906 | | | C18000324: gi\|12229928\|sp\|Q9PTW9\|PSA7_CAR | 6.69E−03 | 2.01 |
| 422431 | AI769410 | Hs. 221461 | ESTs | 4.37E−03 | 2.01 |
| 444654 | AV650572 | Hs. 23440 | KIAA1105 protein | 1.59E−03 | 2.01 |
| 418248 | NM_005000 | Hs. 83916 | NM_005000*: Homo sapiens NADH dehydrogena | 8.07E−02 | 2.01 |
| 437830 | AB020658 | Hs. 5867 | KIAA0851 protein; suppressor of actin 1 | 6.71E−03 | 2.01 |
| 438023 | AF204883 | Hs. 6048 | FEM-1 (C. elegans) homolog b | 2.43E−03 | 2.01 |
| 448770 | AA326683 | Hs. 21992 | likely ortholog of mouse variant polyade | 8.37E−04 | 2.00 |
| 416430 | H60487 | Hs. 159440 | bile acid Coenzyme A: amino acid N-acylt | 1.52E−03 | 2.00 |
| 421360 | AA297012 | Hs. 103839 | erythrocyte membrane protein band 4.1-li | 2.02E−03 | 2.00 |
| 418838 | AW385224 | Hs. 35198 | ectonucleotide pyrophosphatase/phosphodi | 5.60E−06 | 2.00 |
| 425145 | BE242802 | Hs. 154797 | KIAA0090 protein | 3.19E−03 | 2.00 |
| 437672 | AW748265 | Hs. 5741 | flavohemoprotein b5? | 1.19E−03 | 2.00 |
| 439898 | AW505514 | Hs. 209561 | KIAA1715 protein | 3.19E−03 | 2.00 |
| 444743 | AA045648 | Hs. 301957 | nudix (nucleoside diphosphate linked moi | 8.32E−02 | 2.00 |
| 445790 | AV655170 | Hs. 49015 | chromosome 21 open reading frame 35 | 2.55E−03 | 2.00 |
| 451141 | AW772713 | Hs. 247186 | ESTs | 2.04E−02 | 2.00 |
| 454105 | NM_001259 | Hs. 38481 | cyclin-dependent kinase 6 | 3.82E−03 | 2.00 |
| 458389 | H70284 | Hs. 160152 | ESTs, Weakly similar to FPHU alpha-fetop | 4.50E−05 | 2.00 |

Pkey: Unique Eos probeset identifier number
ExAccn: Exemplar Accession number, Genbank accession number
UnigeneID: Unigene number
Unigene Title: Unigene gene title
R1: Wilcoxon rank-sum test p-value
R2: Trimean of Hep C+ Liver over Trimean of Hep C− liver Ratio

TABLE 7B

| | CAT | |
|---|---|---|
| Pkey | Number | Accession |
| 442048 | 750422_1 | AW340495 AI984319 AA974603 |
| 451752 | 10408_5 | AB032997 AI141678 AW978722 BE467119 AI761408 BF727385 AW237035 |
| | | AI934521 BF436248 AI479668 Z40632 AA832081 AW295901 |
| | | BF057835 BE465977 AI621269 BE465983 BF756369 N74056 AI817896 AA716567 AA934774 |
| | | H62600 H09497 BF943762 BE395335 |
| | | BE883333 |
| 421057 | 265006_1 | BE222349 AA830545 BF224127 H99396 N81017 N81016 AI525205 T58283 |
| 407644 | 27910_1 | D16815 AL563603 BG399756 AI935028 N41813 L31785 AL529953 AA428473 |
| | | AA401262 BF802576 AA373868 BE886120 BM479352 T28309 |
| | | N57167 BI551531 BG037160 |
| 444314 | 1027984_1 | AW749625 AW749626 AW749644 AI140497 |
| 432954 | 2159612_1 | AI076345 AI887648 AA572691 |
| 433586 | 32908_1 | BC011194 AW517087 AA601054 T85512 |
| 419175 | 35068_1 | AB018322 BC012480 BI524873 AW665554 AI934469 AI479916 BF096179 BF096162 |
| | | BF096132 AA744972 AI951988 AI858339 BE076331 |
| | | AA886998 AI570585 AI916688 AI678811 AI693109 AI308135 AA669046 AA961064 |
| | | AI018062 H80618 BE221942 R52609 AI915164 AA365626 |
| | | Z44671 BI052776 BF882486 BG286184 AI589558 AA931663 AA534979 AI275392 |
| | | AI273455 R52553 AA829920 H80652 AA360728 F10618 |
| | | AW953666 AW176773 H85527 AA765570 AA081927 BF093262 BG743753 |
| | | AL037576 AA534314 BE814964 BE973713 N49493 BE006634 |
| | | BE006630 AW270037 AA234765 AI334004 BF057179 AI857450 AI341191 |
| | | AI434143 AI917449 AW517207 AA255424 AW008334 AA847572 |
| | | AA994211 AA861901 AA581873 AI580157 AI364363 AW242357 AW235291 |
| | | N55645 AA319869 R36911 AA256551 AW044188 AI203159 |
| | | N49403 F02090 AI187299 AI609644 Z40516 AW952314 |
| 400247 | 2764_1 | BC022339 BC009610 BC010537 X79805 NM_006713 U12979 BM467814 BM450743 |
| | | AU132951 AU137129 BG493425 AV758819 BG708412 |
| | | BG705885 BG702217 AV716638 BG777009 BI545689 BI552153 BM476712 |
| | | BG770858 BG527656 BG528277 BG391388 AV716861 BI602926 |
| | | BG290073 BI667399 BM451469 BI667173 BI602139 BG532171 BI669216 |
| | | BI544727 BG721852 AV716503 AV701327 BM090738 BI492000 |
| | | AI308856 BI544904 AL599813 AV715829 AV716505 AV714587 AV717902 |
| | | BF668072 AV716385 BI461927 BM090954 AV717826 BG503676 |
| | | AV647719 BG501392 BG428433 BE895629 BM313117 AW021050 BG435032 |
| | | BM152910 AA313503 AA872377 BG574714 AV712054 |
| | | AV732696 AA252476 AV712759 AL599643 BE790872 BG654930 W73337 |
| | | AW675377 AV760376 AV725139 AV716379 AA887165 BE830003 |
| | | AW023796 AL599291 AI902948 BG944042 F00781 AA352483 BG217897 |
| | | N33888 AW581924 BG654730 D31410 AA353088 D31288 |
| | | AA295029 H95170 BE935104 AU139980 BG772963 BG776470 BG532512 |
| | | BG105449 BI545421 AV715456 AW386083 BG699714 AL535832 |
| | | AL514940 BG190861 BG210593 AW999254 H95138 AA353863 BE764809 |
| | | N50375 BE091363 BG701255 BI860846 BI832485 BG168150 |
| | | BG028647 BE546301 BG900321 BI909737 BG702363 BG614141 BG611137 |
| | | BG700121 BF031492 N85802 AV715940 N51590 BG993478 |
| | | BE172016 AW893622 |
| 436024 | 138548_1 | AI800041 AA703553 AA984529 |
| 443547 | 137089_2 | AV645808 AA701657 AW271273 AI796734 AI472316 AI017531 AI061178 BF109096 |
| | | AA548964 N83805 AA131648 AA156589 BE708349 |
| | | AW952494 D30877 AV684717 Z24837 F00167 BF576150 T63841 R78995 N87474 |
| 416475 | 1972665_1 | R02750 H58072 T70298 BF367306 R02749 T80873 |
| 408096 | 9330_1 | U61981 NM_002439 J04810 BC004177 BC011817 BC017273 BG876463 |
| | | BG876469 BG876468 NM_000791 BC009634 BE741138 AI830697 |
| | | AW572941 AA489055 AA463881 AI186480 AI803332 AA129465 AA749344 AW572255 |
| | | AA447680 AI184392 H94631 BC000192 BC003584 |
| | | J00140 V00507 AL525560 BG532440 AU130196 AU130635 BG699143 BE295309 |
| | | BG771877 BG497327 AV714645 BE397236 BI458721 |
| | | AU127142 BE252613 AW575796 BG328723 BE250809 BI759776 BM051687 AL040147 |
| | | AA314334 BE780925 AU099092 T29009 BE250162 |
| | | AU139133 AA421716 H99382 BG188773 BE514324 BG199292 BG217128 BG188772 |
| | | BG202122 N44579 BE250719 AW473270 AA382889 |
| | | AU157895 BF907195 J00146 BM475776 AA974401 BF514204 BE743689 AW993728 |
| | | AW364498 BE514508 BG195205 AW069265 BF881506 |
| | | BF928376 BE874450 AI909030 AW051136 AL514516 AW970133 AL514515 AA701106 |
| | | AA516401 AA578546 BF792184 BE566555 |
| 442679 | 31783_3 | BG621493 BI056706 BG496376 R53718 W65356 R79357 BG434247 |
| | | AA357769 AW978686 BG573200 BF132113 BF086709 AA366938 |
| | | D79234 BG494628 AA156754 BG434311 AW978683 AW273417 BM054662 AI799886 |
| | | AI433351 AI160798 AI433742 BF056186 AI281606 |
| | | AW015046 AI439585 AI245530 AI078267 AA807170 AA837395 W61252 AA831085 |
| | | AA287371 AW768354 AA890606 AI302539 AI708575 |
| | | AI673031 AI242260 AW514069 AA283958 AA825452 AI371234 AA425696 AA453422 |
| | | AA827697 R23653 D20240 AA772517 H13802 R66972 |
| | | R79360 R27351 F03379 AA031952 N69504 R33143 R79358 R39136 R38800 |
| | | R15089 R52937 R37502 H01021 R33634 R46551 Z40404 |
| | | BG291052 BG570357 AW391046 BG496872 H23558 |
| 418876 | 121279_1 | AA740616 AA654854 AA229923 |
| 434210 | 54921_1 | AK057015 AI026834 BE857936 AA149091 AI742972 AW439172 AI253168 |
| | | AA255613 BF513175 AI005006 T03406 AW338149 AA836442 |
| | | AA420530 R88566 AI611672 AA433916 AA442855 BF063008 AA812568 AI889706 |
| | | AA715313 AA768539 AA767620 AA665471 AA404380 |
| | | AA665612 BF056442 AA706388 AI650676 AA627448 AI141769 H78227 AW901852 |
| | | H78221 BE701982 BF689273 AA397464 N33072 R60218 |
| | | AW968247 H14833 AA768305 AA043348 R56470 BF739832 R51827 AI474963 |
| | | BG494574 AA149090 BF238154 AI802210 BE000129 |
| | | BF734513 R41964 H21055 R85253 R17705 R40844 BF790218 BG388356 |
| | | BF003037 AA703138 AA377348 W24822 |
| 400133 | 2368_1 | NM_005648 BC013809 L34587 BF103775 BG702618 BG716553 |
| | | BI667090 BG505863 BF983483 BG718195 BI857891 BG501016 BM043599 |
| | | AL521812 BG705730 BI495545 BI495546 BF112248 BM023182 BM023123 AI075173 AW051799 |
| | | BF058224 BI324885 BF436008 AA398446 |
| | | BG822375 BM019558 BM023382 BG164174 N56909 BI467064 BM023464 AI207475 |
| | | BM311415 BG758430 BG758807 AI934826 N90351 |
| | | BG422026 BE910312 AI027778 AI081950 AI360890 BM009115 AI191829 BG759697 |
| | | AI138728 AA399403 AI355589 AI336427 AA868702 |
| | | AA393660 AA025127 BG027630 AA962774 AA631224 BG940967 BE791087 AA573315 |
| | | W81685 AA393525 BG944103 AI339125 AI149864 |
| | | AA977655 N90314 BE612839 BG491847 AI129091 AA461234 AA781198 AA759256 |
| | | AA888954 AA975844 AI184099 AI018025 AA398363 |
| | | AI003331 AI193380 AA626020 AI244476 AI601114 AW135664 AI206607 AW263599 |
| | | AA813219 AI684453 AA878626 AA772222 AI085496 |
| | | AI630226 BG940966 AI022010 AA770649 AA887624 AA491739 AA974295 BG530040 |
| | | AA037091 AA019912 BI160457 H64512 BG503896 |
| 413509 | 1518543_1 | BE145433 BE145419 |
| 429588 | 1396089_1 | AI092511 AI080271 AA455177 AI381859 AI381721 |
| 407347 | 810943_1 | T23514 AI655785 |
| 439195 | 21979_1 | AF086037 H89360 H89546 |
| 413007 | 1343540_1 | BE046662 BE046697 BE046655 |
| 437834 | 294580_1 | BG110129 AW749287 BE535498 AW749299 AW749293 AW749302 AW749298 |
| | | AW749291 AW749294 AW749289 AW749288 AW749296 |
| | | AA769294 AW749297 AW749295 AW749292 BE002573 |
| 427384 | 1367813_1 | AA401778 R92652 T82854 |
| 432600 | 114582_1 | AW973464 AI821831 AI821085 AA640756 AA657438 AA554802 AA650339 |
| 411605 | 10026_3 | BG256892 H10532 N46614 R52610 AW977696 BM460488 W56819 BI042183 BG977498 |
| | | BE767451 BF870009 BG477472 R61137 R14274 |
| | | R20259 R09686 BI838226 BF034269 AA429173 BE741829 AW867495 AI123683 |
| | | AW006831 BE831162 AW452753 AV742717 W86152 |
| | | BF115102 AI633815 BF921562 AA094230 BE092587 W86151 AA526153 AI672156 |
| | | BF914496 R12579 BF852352 AA699780 T57386 |
| | | BF903022 R09933 AA678298 |
| 454067 | 98108_1 | AI809849 AW241782 AA041455 |
| 456034 | 685586_1 | AA136653 AA136656 AW450979 AA984358 AA809054 AW238038 AA492073 BE168945 |
| 432676 | 3503_22 | AI187366 AA618478 AA558869 |
| 428342 | 6712_1 | AK056315 AI015524 AA724079 BI713619 AI377728 AW293682 AI928140 AI092404 |
| | | AI085630 AA731340 BM469629 AW968804 AA425658 |
| | | AA769094 BF446026 AW118719 AI332765 AW500888 AW576556 AI859571 |
| | | AW499664 AW614573 AW629495 AW505314 W74704 AI356361 |
| | | AI923640 AW070509 AI521500 AL042095 AA609309 AA761319 AI381489 H45700 |
| | | AA761333 AW265424 AA909524 AA635311 AA649040 |
| | | AI392620 Z40708 AI985564 AW263513 AA913892 AI693486 AW263502 AI806164 |
| | | AW291137 BI061872 BI059498 AA134476 AW084888 |
| | | AA036967 AW370823 T55263 BI002756 AA489664 BF827261 W74741 BF963166 |
| 449001 | 1968002_1 | AI654836 AI619957 H51659 R99472 R99473 |
| 411656 | 1095889_1 | AW855650 AW855576 AW855577 AW855642 AW855578 AW855621 BG950916 |
| | | BG950923 AW855619 AW855624 |
| 434987 | 121985_1 | AW975114 AI096634 AI767001 AA658364 AW517542 |
| 434280 | 1474904_1 | BE005398 AA628622 AA994155 |
| 414405 | 112689_1 | AL047596 AA393792 AI670731 AI037957 AW874364 AI038137 N62286 AI241379 BE501096 |
| | | AW090696 AI927369 AI669226 AI369437 |
| | | AI371075 AW612409 AI686711 AI183289 AA477717 AI076122 AA635190 AA700984 |
| | | AA781508 D81020 BF575223 AI356183 D79312 |
| | | AI375558 H61111 BG283489 BE090666 BE090664 BE090662 H26545 |
| 407218 | 34845_3 | AK026094 AK024519 BG697329 BI768179 BG680257 |
| | | BE275725 BF945273 AW674728 H62658 BG951138 AA326011 AW993688 W67819 |
| | | BI036861 AA524981 N28558 AW799454 BG286696 BG434856 AA317171 BE548299 |
| | | BF987870 H12577 H87965 BE867572 BG107813 |
| | | BE747295 AL118999 T95439 AA363896 H16012 H27146 H41959 R63045 N49563 |
| | | AA032167 BI917617 H02466 AA207162 AA081975 |
| | | BG749301 T11901 AA091553 BG945206 AA095473 BM172375 |
| 407992 | 101407_1 | AI458141 AI743200 AW418811 |
| 452194 | 90339_1 | AI694413 AW994700 AI912946 N73548 AI082035 AW271652 W24189 W24182 AI719718 |
| | | AA024658 AW810120 AW015394 T79755 AA988043 |
| | | AI709339 |
| 427213 | 507972_1 | AW007211 BE467259 AA960991 AA399252 |
| 433075 | 78_6 | X98248 NM_002959 AU127082 AU143346 BE327147 AI168442 AA426361 BF056336 |
| | | AA418378 AI470560 AI365213 BI032745 BI032986 |
| | | AA969895 AA233278 AW291971 AA418326 AI198417 AI337018 BI032747 AA888000 |
| | | AU099083 AU129681 AL036861 AA418275 BF887436 |
| | | BF767448 AA232975 BI094065 BI859449 AL079651 BF877391 AW902319 BF878798 |
| | | BG281450 AA418268 BG770672 BE890328 BI520437 |
| | | BE387505 AW375004 BI020046 BI033747 BF800905 BF869732 N99710 BF905459 |
| | | BE715637 BE746496 BF918537 BF751392 BF751390 |
| | | BF369123 BG949840 AA984366 H85743 AW593418 AI990196 T92267 AA018359 |
| | | H38111 BM050097 BF692315 AW805907 AI547305 |
| | | AA631091 BF845219 BG949806 T92310 BE277220 |
| 418304 | 1093209_1 | BE883520 BI057842 AA215702 AA215703 AA368006 BE006876 BE066555 |
| 438021 | 45849_1 | AY043361 NM_007013 AL136739 U96113 AL049041 AI750898 AA054855 BF887778 |
| | | BF333173 BI061338 BC015380 BG569062 BM090890 |
| | | AI769300 BE466554 AI091728 AI800660 AA832035 AI342285 AI750899 H66486 |
| | | AA455942 H20821 AI187877 AI417440 AW198099 AI245158 |
| | | T91227 AI373068 AW194829 AA680229 BG291204 AW885601 BF380168 D82380 |
| | | D82098 BF885552 AL050082 BE552267 AI458088 |
| | | AW137654 BM014715 AA418921 AI159868 AA845398 AI743920 AI159888 |
| | | AI653531 AA843952 AI799752 AI360957 R67562 BF445478 |
| | | AA854844 AA552895 C21152 BE614563 BI260650 AU155187 AW169673 AI915801 N29619 |
| | | AA435618 AI361252 AI424627 AI219018 R67514 |
| | | AA548141 AI246669 AA156867 R67561 R67513 AW263860 AA089478 AV653790 |
| | | AA354846 BI560015 AW794547 R52020 H18923 R67555 |
| | | AI302490 AL582101 AI871884 AW029104 AI582326 AV688960 AL040820 |
| | | AI804339 T57931 R67554 AA418232 AA393281 BE076206 |
| | | AW189435 AW979344 AW953494 BF056797 AA310720 N64062 W92065 AI761704 |
| | | AA418824 H89156 H88930 W92048 AV733096 AW007773 |
| | | AI745541 BE504125 AW243111 AI201009 AI187909 R44411 AI795988 |
| | | BF222383 R29593 AA156952 BF029856 |
| 432689 | 11004_5 | AL563022 BC011883 AI625937 BE302243 AI240793 AI886651 |
| | | AL134289 AA877735 AI284399 H80252 AI753853 AA564328 AI075230 |
| | | AA657859 AI269462 F09349 AA653028 T71751 T71812 R58128 AI890218 T71659 |
| 410099 | 16732_1 | AK055674 AW965247 AV751598 AA290926 R53043 AA331387 AK056148 BI917678 BG819395 |
| | | BG911971 BG820167 AI174254 AA348720 |
| | | AA364503 BG714279 AW893230 AA081774 H24222 AV727176 BF875715 AA081630 |
| | | BE000834 AA334880 AL563737 BG029709 W52882 |
| | | AI439658 BE551237 AA283724 BF109530 AI457096 AI805992 BE467736 AA693467 |
| | | AI697593 AI887863 AI167419 AW901980 AW901768 |
| | | BE702179 AA484549 T23811 BE327043 AA716027 AA917004 AA167714 BF339675 |
| | | AA084618 AI418634 T31586 AA436630 AI366472 |
| | | AA706191 AI422304 AI204899 AI041169 AA211402 AW827081 AA788593 T32736 |
| | | AI767935 AA747914 T03534 AW959843 AL119527 |
| | | BE327037 AW901982 AW993370 AW901977 AW902071 W60090 N79906 D52685 T07735 |
| | | BE702069 BE702172 T08671 BE767121 BE767117 |
| | | BE767113 |
| 433891 | 647290_1 | AW182329 AA613792 T05304 AW858385 |
| 425757 | 1228540_1 | AW963347 AA371863 AA363171 |
| 400281 | 9758_4 | BE564506 Z47727 BF028489 BI597545 AA152273 AA730753 |
| | | W07504 M406507 AA316220 AA424683 N35844 N72473 AA442109 W01788 |
| | | AA747605 AA235894 N41438 AA382786 AI906934 AI906935 BF091197 N85572 N75678 |
| | | AV758098 BE719745 BE719744 BE719735 |
| 413645 | 1234345_1 | AA130992 AW969537 AA503835 |
| 423886 | 887218_1 | AW950497 AA332098 AW950499 |
| 408731 | 11725_2 | AF305826 BE350971 AI765355 AW172600 AI310231 BM271766 AI547292 AW612019 |
| | | AI674617 AW138666 AI147629 AI147620 BF857810 |
| | | BF886300 BF885952 BF886303 BF885956 BG565497 BE670834 AA114025 BF886396 BF886928 AV750861 D62864 |
| 443126 | 1057340_1 | AI033503 AW801770 AW801782 |
| 448219 | 22785_8 | AW577769 BE076308 T98501 AW577775 |
| 418259 | 133853_1 | BM310925 AA426110 BM310629 BF434286 AW015091 BF475996 AW118867 |
| | | BE675186 AI688568 AI453594 AW590589 AI652425 AI827969 |
| | | BF056946 AI802866 AI393380 AI476224 AW590639 AW136271 AI458252 |
| | | AI524726 AA843768 AA782158 AI336058 AI097532 AW451563 |
| | | AA459408 AA459633 AA418444 W23607 BG940150 AI493445 AW054729 AI221929 AI868744 |
| | | AA215405 AA766713 AA621546 BF928317 |
| | | BE464132 AI990909 AW271459 AI262061 AA215404 N74332 BG940151 BG952261 AA972115 |
| | | W96315 AA689586 R69057 BF766886 |
| | | BE769254 W05240 |
| 415023 | 37164_1 | BG763540 BI115604 BG325282 AY007096 BE048206 BE048060 AA356949 |
| | | BG469451 BE267760 AI683752 AI702004 BE247172 T47611 |
| | | T47612 BE780300 AA932146 AA652369 AI784281 BM055020 BM023299 AI382709 |
| | | AA159351 BI262171 AI863355 AI674922 AI754389 |
| | | AW295190 BE673083 AW469497 AI949042 AW131720 AI990060 AI623178 BE673420 |
| | | AI863364 AA620354 BF431793 AI056058 AI056059 |
| | | BI258269 BI823911 BG482172 BI261556 BI519838 |
| 412634 | 131633_1 | U55984 AA195883 AA447957 AA393452 AA384799 AW958952 W68044 |
| | | AA197345 AI241933 R59505 W67965 AA826474 AI336006 AA931733 |
| | | AW025414 AI769180 AI355738 AI860952 AA907417 AW408679 AA453139 AA938346 AI279697 |
| | | AA121899 AA400768 AI559827 AA448889 |
| | | N62400 AW236656 AA904207 W79380 R59504 |
| 419970 | 13569_1 | AK055270 BE348291 AI190289 AW612022 AI269506 AI266578 |
| | | AI269675 AW271406 BE879851 BF574163 BI497126 AW903775 BI917368 |
| | | AW150900 BF244813 H79201 |
| 420985 | 455_7 | X94703 NM_004249 AL521205 BF886672 N46403 T87420 Z36855 BF934259 |
| | | BI549507 BG899191 BI517479 BI818431 H39666 |
| 456984 | 29289_2 | W28684 AW959164 AI909150 AW750651 BF055016 AW750639 AI909163 AA358374 |
| | | AV753402 AW968411 BF196253 AI697716 AI656043 |
| | | AW138212 AU146192 AI582306 AI814242 BF036992 AA723154 AW301605 AI378151 |
| | | AI680949 AL589221 AW304035 AA984657 AI290310 |
| | | AI668884 BG149396 AI537796 AA995333 AA402863 AA416859 AI300148 AA889348 |
| | | BE674396 AA740711 AI041113 AA723177 AW976742 |
| | | BF951876 AI909164 AI909181 H08832 R80981 W28685 |
| | | W28701 H18426 R80781 AW842470 |
| 423828 | 886732_1 | AW962569 AA331536 AA331754 AW962568 |
| 411777 | 1104614_1 | BE067593 H49900 BF331057 BF331045 BF331063 BE067552 BF331011 |
| | | BE067819 BF331039 BE067694 AW861997 BF331052 BF331005 BE067800 BE067714 |
| 421828 | 207_22 | AW891965 AW604749 BE080872 R15559 BE177623 AW883520 AW945343 |
| | | AI246167 T07082 AW805679 W96278 AA135796 W32615 |
| | | AW995418 AW801688 BE003837 AW801621 AW385721 AW385742 AW385714 AW604757 W87409 |
| | | AW604738 AW385757 AW580796 |
| | | AW801247 BE003239 BE003183 AA847112 AW580975 AW604760 AW385727 BE164590 |
| | | BE003090 AW362791 AW604759 AW866589 |
| | | AW604758 N44337 AI378548 AW890438 AA077172 AI288683 AA229639 AA091945 |
| | | AW945454 AA063629 AA702504 AW861938 AW894816 |
| | | AW580841 AA094372 T06399 AW885686 BE244086 BE005035 AW861913 AA551773 |
| | | AW858460 AW370926 AW754352 AW889695 |
| | | AW384408 AI907428 BE067491 AW861939 AA248197 AW381373 AW177325 |
| | | AW806879 AA935217 BE067498 BE083742 BE067470 |
| | | AW894935 BE082529 AI248811 BE179917 BE002200 AW607506 AW392889 AW894560 |
| | | AW381360 AI904206 AW863533 C00609 AW381372 |
| | | BE082530 AW898120 BE075323 AW392799 AW601420 AI695314 BE083790 AW858568 |
| | | AW945550 BE177153 AW970506 BE350419 |
| | | AI906919 AW360794 AI906917 AW885979 AW794240 AW945566 AI688683 AI688694 |
| | | AW009660 AW601421 AW360793 BE066524 BE083901 |
| | | AW369847 AW381871 AW935435 AW664582 AW877775 AW838449 BE180466 AW858501 |
| | | BE180464 AI371163 AA778231 AI174991 |
| | | BE011720 AW877776 AW877800 AW877795 T19900 AW866365 AW898099 BE011715 |
| | | BE167842 BE011718 BE011724 AW363639 |
| | | AW878658 AW878662 AW894887 BE082356 AW389211 AW804286 AW610312 AI904717 |
| | | AW610318 AW996909 AW610296 AW901923 |
| | | AW880003 AI762171 AW062582 AW368713 AW062593 AW176663 AW842064 AW842089 |
| | | AW842095 AI243049 AW902074 AW062592 |
| | | AW176664 AW751692 BE087703 AI907439 BE009686 BE172115 BE077030 AW608556 |
| | | AW835577 AI909628 BE077029 AW176241 |
| | | BE077552 BE160370 BE160288 AW835656 AW606765 AW606770 AW835678 AW606758 |
| | | AW606778 AI907484 BE172821 AW606768 |
| | | AW999517 AW844165 BE171738 AW751683 AW610493 BE177484 BE177487 AA090510 |
| | | AW844117 BE173367 AW999878 AI124870 |
| | | BE163472 AW841823 AW379762 AW893297 AI290296 BE089132 AA610287 AW176676 |
| | | AW607622 BE172639 AW893232 AA329629 |
| | | BE089008 BE178350 BE178214 BE063291 AW820236 AW999653 BE089486 BE173126 |
| | | BE171775 BE185787 AA558280 AI174840 AW999112 |
| | | BE218391 BE172734 BE178021 BE172738 BE173324 AW603494 AL036722 R38192 |
| | | R60905 H53721 H41052 AL037917 R37795 AW998972 |
| | | AA767189 AW044272 H50689 AA768399 AA767764 AI087888 H44202 BE222792 |
| | | N90597 W81396 N90615 AI935353 BE501168 F10945 |
| | | AW118215 AI970480 AI627641 AW236081 AA574090 AI627652 AI681913 AI759983 |
| | | N69591 N69276 BE467722 AW392780 BE172467 H92861 |
| | | AI524921 F02989 Z39328 F02705 F01414 T88678 AI215165 H87220 AW374781 |
| 452279 | 11990_1 | AF392454 AK023074 AI884890 AI814455 AW966220 BF736545 AA026021 AA286843 |
| | | AA251918 BG197710 AA026294 AA337356 BC010422 |
| | | AK023226 NM_022776 BM459496 AA769310 AI826460 AU153650 AU160375 AW166211 AW292992 |
| | | BF433538 AI823888 AI684798 AI655985 |
| | | AW770982 AI400454 AI276257 AA639510 AI689818 AW772604 AA807639 AU130298 |
| | | AU132028 BF900889 BF904822 AY007102 AU143256 |
| | | BG621460 AA829630 AI864665 AI084922 AA025234 AI360060 AA766554 AA026295 |
| | | AA825817 AA251762 BE180751 BM464530 AV715833 |
| | | AA779447 AI452519 AW418525 AA435643 D25894 AA435651 AA286844 N64369 |
| | | AI702262 BG288063 BE170545 BM466232 BE299160 |
| | | AA169573 AL567428 BF217285 BE967276 |
| 400189 | 2140_1 | Y08200 NM_004581 BC003093 BE733834 BI753321 BG773890 |
| | | BF091906 BI917541 AI023762 AA587230 BF435086 AI264262 AI687392 |
| | | AI810536 AW589886 AI244419 AA749261 AA535435 AW205689 AI765770 AI765431 |
| | | C02465 AW305347 AI818456 AA322111 AW381845 |
| | | AW381829 AV749407 AA811636 AU159893 AA603065 AA652542 AI468678 R49616 |
| | | AW381863 BE389867 BE182387 BF087771 AA527551 |
| | | AA134051 AA831504 AA134052 AI871759 AW089048 BI913532 AA367709 BG828155 BF093014 |
| 442961 | 60316_2 | BE966247 BE220885 BE467384 BE350135 BE672094 AI811582 AW665254 |
| | | AA772731 AI283601 AA417067 AW197746 AI868357 AI792143 |
| | | AA931120 AI758506 AA843761 BE737582 AW379586 N38812 BG567321 H13257 |
| 422150 | 782_1 | BI086421 BF059136 AW003898 BF446659 AI632891 AI628067 AI703179 |
| | | AI961149 BF111022 AW614154 BE674215 AA687350 AA779426 |
| | | BF591963 AW243344 AI356530 AI492508 AI694049 AI090422 AA465307 AI273387 BE674625 |
| | | AW271971 AA969153 AW468593 AA984014 |
| | | AI817491 AA970258 AI914450 AI018697 AW577591 AW577616 AA382101 AW954455 |
| | | AI867266 AI707995 AI337384 BF208406 BF037100 |
| | | BF223433 BF195517 BE673984 BF224124 BE813387 BG036579 BI553906 AA304995 |
| | | AW361269 AW754160 AW361276 AW361271 AI867118 |
| | | AW805555 AW361284 AW954458 AW958551 BG681507 H79011 AW205696 AW134957 |
| | | AA747667 AW753296 BF939060 AW958549 |
| | | BF910827 BG573750 BG168639 |
| 436262 | 2276_7 | BG028838 AL597498 AW938817 AA707425 BE089516 BE175202 |
| 418733 | 18099_4 | BM473699 N33854 N35083 BE090367 AI306129 AA911920 AA633160 AI302415 AA609954 |
| | | AI365203 AA911919 AW995271 AA234587 |
| | | BF996091 AW956344 AW389533 R60133 |
| 437151 | 282041_1 | AA745618 AW606658 AA745616 AW805449 BF897789 |
| 421272 | 64950_1 | BG819964 R87567 BF062036 BF062044 AW014470 BG057316 AI810506 BE466071 R88291 |
| | | AA704157 C15898 C15173 AA286825 BF594141 |
| | | R84605 H85515 |
| 430068 | 1177709_2 | AA947566 |
| 432474 | 1237469_1 | AW973273 AA584042 AA548798 |
| 446851 | 9025_1 | AL530311 AI929658 BE504156 AW160973 AW007332 BE327718 AI268061 BE219811 |
| | | AW162577 AW139586 AI142112 AI929817 BE348817 |
| | | AI281962 AI694916 AI694425 AA476502 AA911355 AI867757 AL522996 AK025716 BI966713 |
| | | AI823741 BF435119 AI740654 AI377656 |
| | | AI380387 AW612765 AI190300 AI080728 AI425090 AA927634 AI911024 AA826114 AA693577 |
| | | AI003964 AA680381 AI203302 AI004628 |
| | | N75950 AI497985 AI918293 AW168793 AA909369 AA884574 AI191792 AI868413 AI458050 |
| | | AA233857 BM462189 W72450 BG775793 |
| | | AF064843 AI458158 AA568445 AI802171 AA604222 AI393739 BE044639 AI351150 R43836 |
| | | AL523063 AW960720 AA621125 AA587562 |
| | | AA810864 T86692 AA007682 AA055564 AL523983 AA007681 N39693 W04974 AA449174 |
| | | T86786 W76299 R54146 |
| 426221 | MH1660_70 | AF186377 U32581 AA776981 AW473826 AU127861 N24261 W31964 BE702576 BM455665 |
| | | BF371317 BF371314 BF371305 BF371302 |
| 441969 | 2681178_1 | AI733449 AI733386 AA972780 AA988876 AI791798 AI791748 |
| 459436 | 880261_1 | AI908449 AA323121 |
| 425284 | 3834_5 | BM476120 BE672181 AI697653 AA938187 AI280879 AW340123 AI912727 AI081775 |
| | | AI089556 AI191349 AI871604 AI631607 AI890800 |
| | | AI701917 AW771624 AA663041 AI991576 AI160622 AA771763 BE089784 BE089788 AI222942 AW418516 |
| | | AA329211 AI095736 BE550294 |
| | | AA738345 BE218683 BE670548 BG149505 BF061776 D19821 BE005786 BE178892 |
| | | BE005728 BF841237 BE005648 R27634 |
| 411617 | 11546_6 | BF037031 AV752097 AL602446 AI004475 AA312353 N94870 N28394 AA179069 |
| | | BF746111 AA351029 AA096257 AA247994 BM313163 |
| | | AW364986 R25275 AA837053 AA191277 AA809755 AA094345 BM152038 |
| | | BF514649 BM151933 BE769272 |
| 416048 | 1904153_1 | H16413 H16268 H19416 Z43074 |
| 414404 | 19538_5 | BG036308 AW813969 AW579686 BE147476 AW813871 BE147474 BE147589 |
| | | BE147582 AA767716 AW813960 AW813958 BE147583 |
| | | AW813608 AW813612 AW813965 AW813954 BE706154 BF843546 AW813870 BE147801 |
| | | AW813962 R80512 BG681408 AA344564 |
| | | AW370224 BG614838 AW582118 AW582114 AW370227 W16712 BF209670 BG117739 |
| | | BE762802 BE087964 BE888647 R89145 BF342601 |
| | | BI752591 BG435038 AA166732 N79515 BF541709 N79567 BG503029 |
| 437838 | 2512601_1 | AI308202 AI307229 AA769348 |
| 434384 | 132928_1 | M631910 AI928421 AA687629 |
| 446830 | 41421_1 | BC020595 BI488430 BG168023 BE179030 AW294203 BF849776 AA459064 AI917452 |
| | | AW403072 W27419 BF914568 BF798468 AW370558 |
| | | T35055 AW370623 AA399232 AA214221 AW802987 BF902228 AW370622 BF819597 |
| | | AW370567 BF914313 AW954040 BF060706 AA194237 |
| | | T25074 C01285 BI489433 |
| 447657 | 2957064_1 | AI953011 AI953026 AI936839 AI418034 |
| 408138 | 14592_2 | AW374043 BG568336 AW374058 AW961372 AA328028 BF438186 AI268678 AV724415 |
| | | BG029720 AA725816 AI926580 BM310209 AA968470 |
| | | BF430989 AI185775 AW002091 AI969941 AI718705 AI360440 AW008856 AA862294 AA625752 |
| | | AI610753 AA471020 AA723203 AL044614 |
| | | D80637 H77377 AA745928 AA743114 W25004 AI127139 AA953939 AA908426 AI800072 AI417080 |
| | | AW874111 AI439293 AI370639 AI277179 |
| | | AA883338 AA469058 AI200997 W94913 AA047544 W91966 W69102 W84439 |
| | | W52870 W67288 W69103 H98077 AA111874 AI913850 |
| | | AA535740 N92824 AA973639 W68358 BE170126 T81345 N50135 AI567418 W67220 BF437728 |
| 414279 | 5766_2 | BG430995 R53271 Z33587 BE073129 BE073137 R52586 AA137263 |
| | | BG031848 AI983837 AI434776 AI767302 AW236107 AI676204 AI420077 |
| | | AA282786 AI498342 BM310542 BM310847 AI089287 AA398984 AI129279 H79114 |
| | | BF431053 N53529 AI741845 AI333439 AI674468 |
| | | AA137264 AI624731 AA399623 AI968243 AI863637 AW769997 AA618148 AI220925 |
| | | BI492628 R52535 AI474904 H72893 D51042 AI744688 |
| | | AW021691 N38931 N24944 AI537404 AW242437 H99461 Z41183 R52617 BF448803 AI979148 |
| | | R44190 R45431 BG436113 AF236691 H72493 |
| | | AA482849 BF219145 AA625451 BM312732 AA282874 N33821 AW970080 BE780127 R23405 R25093 |
| 416272 | 3292291_1 | AA178882 AA178897 AA179898 |
| 425303 | 1223924_1 | AW958427 AA354785 AA354792 |
| 438980 | 917280_1 | AW502384 AA828822 AI982587 |
| 452480 | 3144986_1 | AI903574 AI903572 AI903526 AI903571 |
| 456721 | 17316_2 | AK027110 AI823404 AI311783 AA552514 AI936370 BF435170 AW468427 |
| | | AA533356 AA779031 AA318991 AA614088 BF868935 R67736 |
| | | BG007848 AA720986 |
| 423983 | 888204_1 | AA333365 AA552870 AA333261 |
| 438141 | 1173217_1 | AA778849 AW946871 AW946782 AW946955 |
| 438041 | 2523562_1 | AI394551 AA812658 AA776285 |
| 416611 | 10454_1 | BC010161 BG576118 AI708321 AI478300 BF111611 AA568308 AI984473 |
| | | AI453629 BI495582 BI495581 AI983169 AI695792 AI695791 |
| | | AA181902 AI376993 AI472805 W26851 AI341559 BF735125 |
| 414496 | 1526_1 | AK058006 BF724822 W65303 AW887764 AW023806 Z25353 AW022095 AA730973 |
| | | W00417 W73819 BF982096 AI927669 AW188021 |
| | | AW770478 AI913512 AA604358 AI697341 AI691028 AI338392 AI079403 H97538 AI144448 |
| | | AI253102 AI051402 AI335900 AI868132 N28900 |
| | | H98465 AF268386 AI799915 AI819228 BE048413 AW304723 AI819923 BF223106 AA155907 |
| | | AW298079 BF055272 BF446804 BF197697 |
| | | W58588 BF197538 AA032180 AA992597 AW590254 AA027824 AI129369 AI131331 AI655843 |
| | | AA932907 AW104493 AI150615 BF110226 |
| | | AW172271 AI312659 AA057312 BE673669 AA722984 AW104985 AI129232 AI078648 |
| | | AI653086 AI703481 AW515897 AI352206 N67076 |
| | | AW297281 AI686162 AA029184 AI610743 AW772016 AI091778 W65401 AI687374 |
| | | AI218085 AI765158 AI018002 AI653068 AI335704 |
| | | AI520850 AW275228 AW275204 AI420247 AA975336 AI697042 AW182235 AA736386 |
| | | AI281682 AW169698 AW263325 BE645834 AI377438 |
| | | AI146706 AA613808 AA716538 BI496247 AA032248 AI698930 AI193399 N70026 |
| | | H86792 AA404489 W61267 BF447230 AA910805 AA150774 |
| | | AA621907 AA902526 AI827634 AW022037 BF059000 BI496246 BG577007 |
| | | BG571077 AA460779 AW816890 AW816893 AW816891 |
| | | AA029183 AA010295 H86850 T83320 BE160823 H12925 N40087 AA096372 |
| | | BE160847 AW816892 AW816889 AW816882 AW816868 |
| | | AW816941 AW816578 AW816940 AW816577 AI431628 AI828113 AA033677 |
| | | AA033654 AA452704 AA317582 AA346971 BF836584 H48669 |
| | | BI861605 BG925200 AA463277 H89048 AA155952 W03252 W01510 W00915 W58589 |
| | | AA164519 N24017 N24622 N27149 N70109 R43771 |
| | | AA010296 W84611 H98889 H88965 AW594424 AA034139 AA065223 N99696 |
| | | BG981481 N94371 AA767970 W47146 N70977 H05510 W61268 |
| | | T90796 AA164518 W47244 AA150883 AA034138 BF338483 F13671 H51317 |
| | | W72716 N21488 AI188071 AI370541 AI754442 AA148524 |
| | | AI749182 W95221 W92522 R20385 |
| 419200 | 9531_1 | BF036043 AW190446 BG194731 AW662036 AI445021 |
| | | BE937550 AW818972 AW393132 AA834685 BF112058 AV721682 H16423 AI270167 |
| | | AI857345 AA937302 AW818444 BE929780 BG498678 BF155010 BI598271 |
| | | BI599811 BE161728 AW578737 AW753711 AW379707 AW381918 |
| | | BG506608 AW028637 AW994240 BF887392 BF790073 AW381624 AV727105 |
| | | BF439618 AA443174 AI018009 N42850 AW573242 AI417258 |
| | | AA463483 AI676131 AI167170 AA836627 AA443828 AW592922 AA235129 |
| | | AA730278 AW439062 AW474332 BI043239 AW474342 BG708553 |
| | | AW362423 BF090028 BE827256 R16550 R39478 R39479 R94368 BG540916 |
| | | BM314745 AA251087 D54231 D55274 BF085805 D31589 |
| | | AW966405 AW994425 D81879 BE093545 AW901107 AA383529 BI021552 |
| | | R56420 N39976 AA573281 H82595 AA234955 BE093539 |
| | | AW367006 BF358697 BF366318 AA663856 BE702099 BF035969 AI267384 |
| | | AI267232 BE348320 AA621574 AA861212 BF083343 BF083341 |
| | | AV745131 D53074 AW954476 AW954472 AA376836 AV724531 D53063 C14928 |
| | | AA093287 AA062638 BG483558 BE940050 AA765954 |
| | | T70171 BE938775 BE940057 D53502 AW373300 AL118798 BM128728 AA193411 |
| | | AW444709 AW952455 AI887612 BF431948 BI496876 |
| | | AI264159 BM128481 AI624657 AI689301 AI969467 AA861685 AA251595 |
| | | AA625761 AA872090 AI826790 AA328366 BE827416 R75951 |
| | | D56918 R68122 BE827384 AL118797 AI184164 AA164411 BI495332 BE858113 |
| | | AI863860 H00660 T69849 AW780389 C14667 BE934995 |
| | | BI018652 R92801 AA164410 H00752 AW373305 AW373299 AW373302 |
| 421297 | 16064_1 | AB033081 AK074324 AW450283 BM128413 BE550945 BE670081 BE674137 AI298087 |
| | | N30050 BF110807 N30072 AI288098 N30068 |
| | | AW861931 AW858575 AI363734 AK025483 BF882045 BM128581 AW771330 BE669540 NM_016376 |
| | | AB037360 AA399607 R94364 BE514643 |
| | | BG684040 BG759578 T83725 AW858561 AW858524 H65293 AW754345 AK025960 |
| | | BM477612 BE071291 BF087041 BG995615 BE071350 |
| | | AI917153 AA436381 BE815318 AK025717 BE857148 AI741138 AW027718 BI789162 |
| | | AI492263 AI690072 AI420179 BI789249 AI637804 |
| | | AW957459 BF222458 AI094328 AW510893 AW295994 AI760430 BE464934 BE220458 |
| | | AI042022 AI653315 BM128472 AW337972 AW515837 |
| | | AI669489 AW664176 AI768730 AW271660 T16381 AI823834 AJ346113 AI242353 |
| | | BF437578 AI273673 AW513498 AI797424 AI674242 |
| | | AI277236 AW731756 AA373089 AW207589 AW467879 AL550999 N30075 BE883669 |
| | | BF155456 AI423079 R60281 T16974 BG676892 |
| | | BF927070 BI962758 BE811485 BE811591 BE811702 BE811600 BE811483 BE811533 |
| | | BF927061 BG008257 BF873368 AL574815 BF946447 |
| | | H70934 BF939868 BF378761 BE082381 BG984527 AA244447 BG983567 AW885571 |
| | | AW293182 AW885607 AA811057 AA610310 AI434406 |
| | | AA514352 H71020 BF740351 BE814815 N66648 AA737686 AI025403 AA255490 |
| | | N26754 AI094233 AA629548 AA716234 BF343632 |
| | | AW363633 H62663 N40757 N55959 AW858569 AW604731 N40773 N40777 T83895 |
| | | AI905899 BG007487 BF820299 R60231 R60787 |
| | | AA456130 AW383736 BF087045 BG995619 |
| 439046 | 2580645_1 | AA829660 AI687296 AA947354 |
| 440638 | 371165_1 | BG009500 AI376551 AA897445 T87714 |
| 434747 | 117643 _1 | AW976537 AI033582 AA837085 AA745261 AA648395 |
| 453929 | 97058_1 | AA045461 AA284145 AI494193 AI469576 AV700262 BF222563 AW190054 AI263834 AA040074 AA045137 |
| 455834 | 1518503_1 | BE145364 BE145429 BE145418 |
| 421921 | 100_2 | BC011987 AA976353 AA858127 AI370017 AW150562 N22469 AI823318 T39763 AW594422 |
| | | T39771 T39737 T39788 AA857399 T39764 |
| | | T39745 NM_012456 AF150089 BI835353 BG683054 BM021899 AI693046 |
| | | AI350334 AA609007 AA405045 AA412628 AW175775 AA035114 |
| | | BE729995 AV755393 AW027107 AI500393 H83363 F27315 BI966096 BG338889 BF342663 |
| | | AI097243 BE047615 AA628513 AI004714 |
| | | AA147779 AA993774 AA670296 AA845320 AA908522 AI002166 W61055 BE349980 |
| | | AW205379 R80128 AA383242 AI016394 AA035115 |
| | | AI309333 AI568254 AI094665 W04770 W24624 F27773 AA378373 AA353029 R50427 |
| | | BF341287 R99348 BF374303 N75319 AA563593 |
| | | BF933630 |
| 400120 | 133_1 | BC014030 NM_004068 BC004996 AK057883 D63475 BM468205 |
| | | BG386792 BG750447 BG575842 BG479084 BG741027 AU118129 |
| | | BE901043 AU141281 BG825395 BG338276 BE396231 AU121493 AU131489 BF341132 |
| | | BG335659 BI769251 AU142779 AU124483 BG480828 |
| | | AU121353 BG702326 AU137866 BG759046 BI460601 BE887290 BG750415 BF127710 |
| | | BG757819 BI160266 AU122086 BG824046 BG744180 |
| | | BE884943 BM006610 BG702599 BI870749 BF307033 BI334771 BG480606 AU142599 |
| | | AU141336 BM018563 BI335565 AU138308 BE391241 |
| | | BG774488 BE278025 BG717959 BG706920 BE312567 AU138300 BE303034 BE303016 |
| | | AU139252 BE336797 BG470940 BE336735 |
| | | BF305197 AU134288 AU139907 BE261245 BE208718 BG761912 BF212890 AW732692 |
| | | BM011258 AW247629 AU136696 BF530078 |
| | | BF204146 AL048752 BG328927 BE388385 BE260122 AU138789 BE253465 BF733914 |
| | | AW249415 AW239535 BE311791 BE256236 BF529742 |
| | | BG770465 AW245777 AW245813 BE296677 BE266852 BE168115 BE396596 BE280057 |
| | | BE168229 AI750820 AU134137 BF792191 AI272215 |
| | | AI907348 AW238875 BF805152 BF568397 BE712727 BE081443 BE001805 BF724536 |
| | | BF744705 AW247385 BE796369 AU133759 AV705142 |
| | | BE794402 BM465821 BG281284 AW384831 BM450689 AU134125 BE311650 BF356318 |
| | | BM462831 BG420555 BE749127 AU134590 |
| | | BM019438 BC013796 BG761223 BG122058 BE872076 BG748496 BG821374 BE619159 |
| | | BG423244 AU137110 AU127210 BE270081 |
| | | AA496860 AA351380 AA356303 AU099781 AA355912 AA371411 AA325535 U36188 |
| | | AL569574 AL577204 AL525543 AL567342 AL567334 |
| | | AL567122 AL536527 AL567098 BG681585 BG824951 BI870652 BI225855 AW393878 |
| | | BG750632 BI223803 BE877552 BE270473 BE389392 |
| | | BE779021 BM017845 BI117816 BE396513 BM049006 AW393945 BE786941 BE267724 |
| | | BE562981 BE314236 BF744102 BM019781 BF806063 |
| | | AA909846 BF924341 AW361460 AA425174 BE940557 BF761585 BF931276 BF934886 |
| | | W68597 BF933977 BI035906 BE836064 BF837576 |
| | | BI018368 AA205908 H54612 R88902 BE812330 BE932300 BF924562 AW805376 BE769859 |
| | | R87381 M371901 BE838855 AA326381 W56191 |
| | | AA341231 AA464093 AL575977 AL518650 AL547393 BG338327 AL517563 AL536921 BG207096 |
| | | BG207589 BG199290 BG220159 BG744842 |
| | | BG104730 AA555035 AA618009 AA861062 AA610582 AW245418 AI040983 AA521380 |
| | | AW245455 AU147292 AU155236 BE620286 |
| | | AW250767 BF888236 BE620819 AU154343 AU150827 AU148334 AW069495 AA701091 |
| | | AU155225 AW250019 AA666235 AU147764 |
| | | AA449739 BF907598 AW043731 AU144390 AI924565 BE300631 AW874021 BE208088 |
| | | BF732773 BF594057 AI159873 AU143930 N24100 |
| | | AI052499 AI989370 AI366151 AI139248 AU144776 AA838250 AI095433 Al269227 |
| | | BF811358 AU151352 AA477554 AI131290 AI049797 |
| | | AU157349 AA620559 AA581533 AI338299 AA402755 AI050079 AU157614 AI280691 |
| | | AA706590 BE206615 AA861056 AU153214 AU155712 |
| | | AW069554 AI081124 AU155846 AI081123 AA719876 AI754976 AI027763 AA723095 |
| | | BI518782 AW050706 F37521 R79062 AA565530 H39826 |
| | | AA404670 AU158884 AI139682 AA513633 AI283202 AA651856 AI623515 AA427413 |
| | | AW248474 BF841432 BM453246 AI302390 AA808269 |
| | | AI249357 AA132775 AA716732 AA479932 AI198296 AI208674 AI088433 F36977 |
| | | AA496899 AW615762 AA602541 AA341161 D58785 T15399 |
| | | AI286077 W69152 F28418 AI272156 AA338034 AA568455 BE206121 AI080033 |
| | | AI699680 AA861255 AI953465 AI613240 AL581773 AL531577 |
| | | BF526361 AA657809 AW152670 BE621523 AW078705 AW673719 AA102613 |
| | | AA195604 R72209 AA402208 AA404305 N24999 AI963535 |
| | | D54741 BI461415 BM475959 BI260926 BI116213 BE280120 BE940258 BE280044 |
| | | AI905744 BG001474 BE937718 BF799283 BI091621 |
| | | BG421006 BG289235 BF736825 BI253429 BG170064 R72512 T92815 BF931257 |
| | | BM455183 BG033362 BG574220 N31395 BF739185 |
| | | AA371995 BE879011 BI198754 BF378989 T12266 BM019421 BI194570 BF378992 |
| | | AW996595 BI858649 W94605 W61345 AA122384 |
| | | AA171923 T92736 AA761504 AI819039 AI439358 AL517777 AL565919 AA622001 |
| | | AA642695 AA704144 AA081465 AA070621 AA375562 |
| | | AA700011 AA926863 W37310 AL566236 BF677809 BG760021 AW361433 BE828605 |
| | | BE268449 BF805977 BG292452 BF981071 BF217108 |
| | | BF928698 R33993 AW882841 AI857453 AW078733 AI433035 AI018103 R90927 |
| | | AA804720 AA551734 R33835 H19741 R78754 BF930494 |
| | | BG109583 AA631926 BE834008 BG996533 |
| 437580 | 1240047_1 | AW976343 AA761075 AA983906 |
| 433162 | 2167905_1 | AI742311 AI025842 AA578843 |
| 412240 | 8235_1 | BF963346 BI460763 BI599382 AI188089 AY055003 AW959185 AA187681 AI692282 AU153608 |
| | | BI493898 BI493899 AI797349 BE550679 |
| | | AI701137 AA744561 AA082682 BE218816 AW511501 BF447881 AI859101 |
| | | BF059295 AI914038 AI346564 AI685003 AI676076 AI125336 |
| | | AW469637 T10225 AI831296 R53451 AI453440 AA983739 AW470873 AI348290 |
| | | BE857670 D55901 Z43908 T34429 F07305 AK074340 |
| | | AL538118 BG201484 AI334192 T35535 AW176751 BI496132 BI496133 AA469961 |
| | | H72176 BF326265 AI770016 AI693177 BF223634 |
| | | BF963661 BF962265 BI034894 AI475851 N69071 AI910707 BE219038 AI918036 |
| | | BE670589 BM193515 AI338497 AA805525 AA487625 |
| | | AA830336 AA749368 AW104323 AI628055 AI932332 AI272788 AI536849 AW162414 |
| | | AW161923 T23854 AA610763 AA912188 AW339028 |
| | | Z39946 T10224 F03171 AI205478 AI638791 F02580 F01551 AW207551 AA421030 |
| | | BE246012 BI034937 BI035373 BF939581 H19984 |
| | | BE867247 H92677 N55988 BF957332 H18615 R16442 BF956229 BF961886 R12698 |
| | | AA101186 AA365932 R53452 BF062714 BF959364 |
| | | H41634 AW086187 H19985 BF054881 F02581 H18616 H41527 AW003446 BE243443 |
| | | H92276 AW954002 BI670132 |
| 447881 | 44623_1 | AK074291 AW293424 BE676135 AI832125 BE019146 |
| | | BE465019 AI761124 AA617778 AI279232 AW575897 AI672039 F28618 BF924261 |
| | | AA722184 BF934174 BE004328 AV749301 BE880282 BI019798 BI019389 BF928776 |
| | | AW813409 AV726604 AA077560 BE272975 BF949119 |
| | | AW814195 BE879126 AI697926 BF594155 BE205787 BF063513 N35828 AI948557 |
| | | AI433839 AI379679 BG056182 AI589094 N23123 |
| | | AA588805 AW316581 AI080272 AI421980 AI493318 BF194830 N87590 AA495993 |
| | | N32996 AA699844 H96845 H96592 N28741 BI035539 |
| | | BF747723 BF171066 W01350 H05495 AI243785 Z39622 AA887432 AI350659 R46102 |
| 411590 | 2329293_1 | T96183 T64070 AA094134 |
| 414588 | 296561_1 | AA495793 AA302905 AA149783 BI059949 BF916498 BF924146 AW838065 BF990024 AW837915 AW837899 AW837927 |
| | | AW837919 |
| | | AW837903 AW838051 |
| 446126 | 610_2 | BF946219 BF946218 BF851494 AL536879 AA457150 AI590194 AI582629 |
| | | AA464515 AA916242 M337109 AA336509 N46906 AA336322 |
| | | AA336407 AA337222 AA319240 BI026817 BI027058 AL536880 AI693827 AA651730 |
| | | AI701013 BM068789 AW339506 AA293021 BF891108 |
| | | AI458885 AW361203 AW974652 AI761251 AI655763 AA628063 BE047125 |
| | | AW085916 AI129587 N52070 AW172361 AA052951 AW085909 |
| | | AI000008 AA962570 AI371342 AI364207 AA464514 AI962506 AI824603 |
| | | AW376300 AA058439 AW361192 AV656660 N50282 BF820514 |
| | | BF891008 H40784 BF891112 BE708029 AW043567 AA056762 |
| 438619 | 35124_1 | NM_016575 AB032773 AI765521 BF593742 AI497757 AI761233 AW467938 BF000670 |
| | | AI818496 N24761 AL043306 BF476138 BF593836 |
| | | AA132787 AI147248 AI086795 AA151317 T95298 AW083548 AA058371 N27951 |
| | | AI769860 AI784548 AW205506 AI800679 AI041733 AI459902 |
| | | BE327641 AI865829 AI254736 AI302433 AI744176 AI241825 AA027842 AL524933 |
| | | AL524932 BF947764 BF340737 BF948700 BG996395 |
| | | N53455 N21027 AI127616 N35901 M682443 AA678249 AA719371 M132582 T15981 |
| | | H99958 N40717 AW959402 AI267251 BF909329 |
| | | AI142035 T95379 H29420 R59632 H17318 H17331 H29327 R40829 R43395 R59573 |
| | | AI749561 R56599 H16755 AI694500 AA027907 |
| 411468 | 1085621_1 | AW857472 AW847920 AW857357 AW857470 AW857354 |
| 432709 | 1870037_1 | H17238 AA563739 F05559 F06116 |
| 414883 | 8371_2 | AF274943 BG494894 AI719075 AA908783 AI935150 AI422691 AA910644 AA583187 BM272167 AI828996 |
| | | AA527373 AW972459 AI831360 |
| | | AA772418 AI033892 AA100926 AU154749 AI459432 AI423513 AI094597 |
| | | AA740817 AI991988 AI090262 AI312104 BI256707 AA459522 |
| | | AA416871 AI075239 AI339996 AA701623 AI139549 AI336880 AA633648 |
| | | AI989380 AI362835 AA399239 AI146955 BF514270 N92892 |
| | | AI348243 AI278887 AA459292 AI494230 BF507531 AI492600 M962596 AW613002 |
| | | AA293140 AA235549 BF108854 AA954344 N49682 |
| | | AI457100 AW589407 AW300758 BE220715 BE220698 BE569091 BM009647 |
| | | BF900351 AI537692 AI203723 AI857576 AA584410 AW371667 |
| | | BM172363 |
| 449901 | 1269988_2 | BI227033 AI674072 |
| 426295 | 510_1 | BE880923 BG390191 AW470082 AW014585 AI423255 BI714731 BG054894 AW780248 N31683 AW664132 |
| | | AW467353 AI983152 AA617918 |
| | | BF447795 AI088357 AA807328 AA576970 AI741153 AI755003 AI474016 |
| | | AI422030 AI348114 AW997085 BM271753 AI363147 BM311311 |
| | | AM146640 AI246771 AW512619 AI359020 BG054897 AI292234 AI215830 |
| | | AI283836 C06205 AW503423 AW272680 N33205 AW873021 |
| | | AA070724 AI753886 AW192487 AI087151 AA658909 AI346368 AI335677 |
| | | AA825442 AW440066 AW131357 AW513210 AI082314 AI085455 |
| | | BE551404 AA780704 AW008596 AI796964 AA917471 AI400531 AA668626 |
| | | N72207 AI306482 AW440562 AI084687 AA347280 AA063536 |
| | | BF477389 AI241662 AA931543 AA484310 AA812486 AI032216 AA665779 |
| | | AI916336 AI350590 BF198106 AI433377 AI300638 AI672626 |
| | | AI282741 AI351487 AW105544 AA973627 AW517914 AA715424 AA508454 |
| | | BF334080 AI274618 AW367201 AW572619 AW469088 AA382095 |
| | | AI368364 AI146934 AI357180 AI361181 BI911347 BI871044 AA136325 |
| | | BF084010 BF084007 AA335788 AI920878 AA809614 BE932941 |
| | | AI678261 C75308 AI148479 BE178174 W88513 BM013627 |
| 455705 | 77478_2 | BF971018 BE513812 AA133359 AW581719 BF434402 AL600619 BG699731 BI551395 AW027136 |
| | | AW055130 BF939512 AI076048 H18584 |
| | | AW161061 AA864334 AI816101 BE049456 AW044012 AA954079 AI274682 |
| | | AI370526 AW131990 AA853195 AA853191 BG118295 AA761620 |
| | | BG705371 BF355591 BF336596 AA360497 H28072 BG198352 AW364709 |
| | | H40926 H44214 AA836538 BI059563 |
| 424571 | 9758_1 | BE379766 AW152643 AI803450 AI564343 AI092711 AI140525 AW152156 AI620740 AI554689 |
| | | AI161209 AI290242 AI339745 AI374611 |
| | | AI347388 AI858296 Al140529 AI366124 AA493912 AA406235 AA493889 |
| | | AI057160 AW022264 AI097277 AM144126 AI080051 AA983529 |
| | | AA860507 N53469 AA843767 N81163 N70628 AA424577 AA983537 BF003004 |
| | | AA626688 AA235977 AI057152 AI095366 AI095356 AA458646 |
| | | AW194479 AA150439 AI375272 AW571777 AI359198 AA993793 BE614394 BE738239 |
| | | AA127883 AI034344 T59504 D81608 AA908704 |
| | | AW051665 AA382785 AA307208 N24639 AI370715 BE244980 AA548596 AW449675 |
| | | AM91008 BF223749 N70752 N22266 AI191012 |
| | | AA028001 AI419106 BF215661 BF591548 BG942356 AI474968 BE858217 BF793358 |
| | | AV756758 BG483603 AI093724 BF693395 BG545345 |
| | | AI744294 T59549 AA811773 BG499757 |

Pkey: Unique Eos probeset identifier number
CAT number: Gene cluster number
Accession: Genbank accession numbers

TABLE 70

| Pkey | Ref | Strand | Nt_position |
|---|---|---|---|
| 403790 | 8084957 | Minus | 87826-87947,89835-90002 |
| 405102 | 8076881 | Minus | 120922-121296 |
| 402727 | 9211324 | Plus | 54596-54777 |
| 405268 | 4156151 | Minus | 24404-24521 |
| 400517 | 9796686 | Minus | 49996-50346 |
| 405141 | 8980911 | Plus | 99861-100054 |
| 403738 | 7212067 | Plus | 38434-38562,56876-57007, |
| | | | 59789-59876,6071 |
| 403904 | 7710675 | Minus | 8129-8261,41911-42053 |
| 402737 | 9212184 | Minus | 13358-13552 |
| 401091 | 9958240 | Plus | 94760-94898 |
| 402507 | 9797889 | Plus | 118979-119086 |
| 402964 | 9581599 | Minus | 46624-46784 |
| 406423 | 9256411 | Plus | 165600-165824 |
| 406038 | 8389537 | Plus | 37764-37877 |
| 401649 | 9090641 | Plus | 80229-80762 |
| 400475 | 9957990 | Minus | 64771-64887,67396-67563 |
| 403575 | 8101156 | Minus | 81961-82068 |
| 401016 | 8117441 | Plus | 126234-126359,128050-128236 |
| 405689 | 4508117 | Minus | 92558-92698,94282-94382, |
| | | | 97977-98180.9920 |
| 403809 | 8568861 | Plus | 33910-34129,34583-34862 |
| 403671 | 7272159 | Plus | 104461-104701 |
| 402041 | 7770639 | Plus | 69353-69454 |
| 403478 | 9958258 | Plus | 116458-116564 |
| 403027 | 7670575 | Plus | 60696-60932, 61362-61521 |
| 400471 | 9931670 | Minus | 105629-105760 |
| 404433 | 7407979 | Minus | 124561-124764 |
| 401898 | 8570008 | Minus | 72013-72132, 72400-72487 |
| 402836 | 8745058 | Minus | 96756-96941 |
| 403330 | 8516153 | Plus | 116558-116698 |
| 403743 | 7652003 | Minus | 136463-136646 |
| 406274 | 7543787 | Plus | 932-1123 |
| 401197 | 9719705 | Plus | 176341-176452 |
| 404676 | 9797204 | Minus | 56167-56342, 58066-58189, |
| | | | 58891-59048, 6045 |
| 401635 | 7145001 | Minus | 51895-52764 |
| 403976 | 7657840 | Plus | 24755-24969 |
| 401928 | 3873182 | Plus | 54932-55070 |
| 401952 | 3319121 | Minus | 53770-53979 |
| 405117 | 8096945 | Minus | 109169-109866 |
| 400557 | 9801261 | Plus | 208453-208528, 209633-209813 |
| 403920 | 7710868 | Plus | 40312-40490 |
| 403496 | 7523883 | Minus | 140940-141126, 144920-145123 |
| 405122 | 8137462 | Minus | 95449-95646 |
| 403320 | 8318526 | Plus | 127978-128124 |
| 405270 | 4156145 | Minus | 3952-4123, 6886-7010, 8541-8728 |
| 405688 | 4508117 | Minus | 88702-88899 |
| 401627 | 8575953 | Minus | 72828-73029 |
| 403576 | 6862645 | Minus | 73475-73940, 74079-74207 |
| 403742 | 7212067 | Plus | 84805-85525, 91096-91227, |
| | | | 93970-94057, 9523 |
| 405086 | 8072509 | Plus | 73664-73841, 74081-74217, |
| | | | 74610-74779, 7492 |
| 402929 | 8217647 | Minus | 51862-52032 |
| 404240 | 5002624 | Minus | 116132-116407, 116653-116922 |
| 400906 | 9966290 | Plus | 112863-112989, 120162-120286 |

Pkey: Unique number corresponding to an Eos probeset
Ref: Sequence source. The 7 digit numbers in this column are Genbank Identifier (Gl) numbers. “Dunham, et al.” refers to the publication entitled “The DNA sequence of human chromosome 22” Dunham, et al. (1999) Nature 402: 489-495.
Strand: Indicates DNA strand from which exons were predicted.
Nt_position: Indicates nucleotide positions of predicted exons.

Table 8A lists about 254 genes down-regulated in Hepatitis C positive liver tissues compared to Hepatitis C negative liver tissues. These genes have the potential to be diagnostics and/or prognostic markers for Hepatitis C infected liver tissues. They may also provide clinical information on Hepatitis C infection and pathology. They may also be potential targets for therapeutic drugs and/or treatments. These were selected from 59680 probesets on the Affymetrix/Eos Hu03 GeneChip array such that the Wilcoxon rank-sum test p-value between the 2 groups was less than 0.10, the ratio of the “weighted average” of Hepatitis C negative liver tissues to the “weighted average” of Hepatitis C positive liver tissues was equal to or above 2.0, and that the differences between the same 2 groups was equal to or above 30.0. The “weighted average” of the Hepatitis C negative liver tissues was set to the trimean of several different Hepatitis C negative liver tissues. The “weighted average” of the Hepatitis C positive liver tissues was set to the either 10 or the trimean of several different Hepatitis C positive liver tissues, whichever value was greater to eliminate ratios with a denominator of zero or less.

TABLE 8A

| Pkey | ExAccn | UniGene | Unigenen Title | R1 | R2 |
|---|---|---|---|---|---|
| 451831 | NM_001674 | Hs. 460 | activating transcription factor 3 | 2.25E−04 | 23.90 |
| 450912 | AW939251 | Hs. 25647 | v-fos FBJ murine osteosarcoma viral onco | 7.80E−06 | 23.32 |
| 414559 | AV656184 | Hs. 76452 | C-reactive protein, pentraxin-related | 9.77E−04 | 20.48 |
| 447078 | AW885727 | Hs. 9914 | ESTs | 2.93E−02 | 14.42 |
| 416188 | BE157260 | Hs. 79070 | v-myc avian myelocytomatosis viral oncog | 8.41E−04 | 12.75 |
| 451029 | AA852097 | Hs. 25829 | ras-related protein | 9.37E−03 | 10.77 |
| 434078 | AW880709 | Hs. 283683 | chromosome 8 open reading frame 4 | 1.93E−03 | 10.32 |
| 442941 | AU076728 | Hs. 8867 | cysteine-rich, angiogenic inducer, 61 | 9.18E−05 | 10.10 |
| 405278 | | | NM_002864: Homo sapiens pregnancy-zone pr | 5.19E−03 | 8.45 |
| 428106 | BE620016 | Hs. 182470 | PTD010 protein | 5.03E−04 | 7.47 |
| 446066 | AI343931 | Hs. 149383 | ESTs | 2.01E−03 | 7.30 |
| 407173 | T64349 | | gb: yc10d08.s1 Stratagene lung (937210) H | 4.77E−03 | 6.90 |
| 414220 | BE298094 | Hs. 101150 | gb: 601118231F1 NIH_MGC_17 Homo sapiens c | 5.74E−02 | 6.65 |
| 431319 | AA873350 | Hs. 302232 | ESTs | 1.19E−02 | 6.52 |
| 416434 | AW163045 | Hs. 79334 | nuclear factor, interleukin 3 regulated | 2.63E−02 | 6.50 |
| 448607 | AL042506 | Hs. 21599 | Kruppel-like factor 7 (ubiquitous) | 4.36E−03 | 6.50 |
| 420101 | AW500529 | Hs. 95180 | KIAA0767 protein | 6.53E−04 | 6.49 |
| 414327 | BE408145 | Hs. 185254 | ESTs, Weakly similar to T24435 hypotheti | 3.13E−04 | 6.32 |
| 446584 | U53445 | Hs. 15432 | downregulated in ovarian cancer 1 | 5.39E−02 | 5.82 |
| 404501 | | | nucleoside phosphorylase | 6.12E−02 | 5.70 |
| 420548 | AA278246 | Hs. 920 | ESTs | 7.62E−03 | 5.68 |
| 409233 | AK002001 | Hs. 51305 | v-maf musculoaponeurotic fibrosarcoma (a | 1.09E−04 | 5.55 |
| 427509 | M62505 | Hs. 2161 | complement component 5 receptor 1 (C5a I | 7.95E−03 | 5.48 |
| 400425 | AY004252 | Hs. 287385 | PR domain containing 12 | 1.91E−04 | 5.40 |
| 401149 | | | Target Exon | 2.04E−05 | 5.35 |
| 427557 | NM_002659 | Hs. 179657 | plasminogen activator, urokinase recepto | 3.33E−05 | 5.26 |
| 449986 | AW864502 | | gb: PM4-SN0016-120400-004-b12 SN0016 Homo | 3.61E−02 | 5.25 |
| 419299 | AI311085 | | hypothetical protein FLJ22573 | 3.50E−03 | 5.15 |
| 450335 | BE218355 | Hs. 201781 | ESTs, Weakly similar to B34087 hypotheti | 3.06E−03 | 5.15 |
| 417752 | C15737 | Hs. 269386 | ESTs | 5.87E−04 | 5.07 |
| 423053 | BE312679 | | gb: 601148138F1 NIH_MGC_19 Homo sapiens c | 1.81E−04 | 5.02 |
| 448871 | BE616709 | Hs. 159265 | kruppel-related zinc finger protein hcKr | 2.22E−03 | 5.02 |
| 408278 | AW876813 | | phosphoglycerate dehydrogenase | 2.66E−04 | 5.00 |
| 431326 | AW970580 | Hs. 198689 | KIAA0728 protein | 6.12E−02 | 4.92 |
| 436068 | AK000038 | Hs. 300979 | ESTs, Weakly similar to I38022 hypotheti | 5.92E−03 | 4.85 |
| 409795 | AI934808 | | ESTs, Weakly similar to T46338 hypotheti | 2.32E−03 | 4.84 |
| 430070 | AF197927 | Hs. 231967 | ALL1 fused gene from 5q31 | 2.94E−05 | 4.83 |
| 411929 | AA098880 | Hs. 69297 | ESTs | 1.14E−02 | 4.75 |
| 402273 | | | Target Exon | 2.11E−03 | 4.72 |
| 453196 | AW003567 | | ESTs | 3.50E−03 | 4.72 |
| 418303 | AA215701 | Hs. 186541 | ESTs, Weakly similar to I38022 hypotheti | 2.63E−02 | 4.70 |
| 455510 | AA422029 | Hs. 143640 | ESTs, Weakly similar to hyperpolarizatio | 6.12E−02 | 4.65 |
| 423720 | AL044191 | Hs. 23388 | hypothetical protein DKFZp434F0318 | 1.13E−03 | 4.63 |
| 402517 | | | Target Exon | 1.13E−03 | 4.61 |
| 454598 | AW809716 | | gb: MR4-ST0124-241199-026-h09 ST0124 Homo | 3.06E−03 | 4.40 |
| 448429 | D17408 | Hs. 21223 | calponin 1, basic, smooth muscle | 1.89E−02 | 4.35 |
| 429258 | AA448765 | Hs. 184252 | gb: zx10e09.r1 Soares_total_fetus_Nb2HF8_{—} | 7.00E−03 | 4.34 |
| 412176 | AW898334 | | gb: RC3-NN0070-270400-011-f02 NN0070 Homo | 7.60E−04 | 4.27 |
| 441269 | AW015206 | Hs. 178784 | ESTs | 1.29E−02 | 4.22 |
| 458867 | AW995393 | | gb: QV0-BN0042-170300-163-g12 BN0042 Homo | 1.16E−06 | 4.22 |
| 417732 | R36065 | | gb: yg69h06.r1 Soares infant brain 1NIB H | 9.14E−05 | 4.13 |
| 444863 | AW384082 | Hs. 104879 | serine (or cysteine) proteinase inhibito | 6.12E−02 | 4.13 |
| 415477 | NM_002228 | Hs. 78465 | v-jun avian sarcoma virus 17 oncogene ho | 7.23E−04 | 4.12 |
| 415994 | NM_002923 | Hs. 78944 | regulator of G-protein signalling 2, 24 k | 9.78E−04 | 4.05 |
| 417551 | AI816291 | Hs. 82273 | hypothetical protein | 4.30E−04 | 3.98 |
| 426006 | R49031 | Hs. 22627 | ESTs | 3.61E−02 | 3.95 |
| 401553 | | | Target Exon | 8.32E−02 | 3.95 |
| 432877 | AW974111 | Hs. 292477 | ESTs | 9.36E−02 | 3.92 |
| 410052 | AA525225 | Hs. 334630 | Homo sapiens cDNA FLJ14462 fis, clone MA | 1.14E−02 | 3.92 |
| 459476 | BE185844 | | gb: IL5-HT0731-110500-087-c08 HT0731 Homo | 7.31E−03 | 3.92 |
| 404958 | | | Target Exon | 9.30E−04 | 3.90 |
| 431394 | AK000692 | Hs. 252351 | HERV-H LTR-associating 2 | 4.76E−03 | 3.88 |
| 403324 | | | C2000428*: gi\|7705383\|ref\|NP_057536.1\|GC | 1.34E−02 | 3.88 |
| 447744 | AA313230 | Hs. 19413 | S100 calcium-binding protein A12 (calgra | 1.69E−02 | 3.88 |
| 447802 | AW593432 | Hs. 161455 | ESTs | 1.83E−03 | 3.87 |
| 424946 | M64572 | Hs. 153932 | protein tyrosine phosphatase, non-recept | 5.38E−02 | 3.85 |
| 401913 | | | ENSP00000249158*: CDNA | 8.32E−02 | 3.84 |
| 430389 | AL117429 | Hs. 240845 | DKFZP434D146 protein | 1.10E−02 | 3.83 |
| 441188 | AW292830 | Hs. 255609 | ESTs | 3.14E−02 | 3.82 |
| 449236 | AJ403126 | Hs. 26373 | Homo sapiens cDNA: FLJ23449 fis, clone H | 4.73E−02 | 3.82 |
| 446052 | AA358760 | Hs. 95893 | gb: EST67699 Fetal lung II Homo sapiens c | 2.16E−05 | 3.81 |
| 431861 | AA521072 | Hs. 292128 | ESTs | 5.29E−04 | 3.76 |
| 411993 | AA099329 | Hs. 151764 | ESTs | 2.54E−02 | 3.75 |
| 449335 | AW150717 | Hs. 345728 | STAT induced STAT inhibitor 3 | 2.11E−03 | 3.73 |
| 403794 | | | Target Exon | 1.08E−03 | 3.71 |
| 409840 | AW502122 | | gb: UI-HF-BR0p-ajr-c-08-0-Ul.r1 NIH_MGC_5 | 4.51E−05 | 3.67 |
| 410057 | R66634 | Hs. 268107 | multimerin | 1.39E−02 | 3.65 |
| 456525 | AW468397 | Hs. 100000 | S100 calcium-binding protein A8 (calgran | 4.57E−02 | 3.65 |
| 447572 | AI631546 | Hs. 287331 | zinc finger protein ZNF286 | 1.45E−03 | 3.65 |
| 449785 | AI225235 | Hs. 288300 | hypothetical protein FLJ23231 | 1.69E−02 | 3.65 |
| 412568 | AI878826 | Hs. 74034 | caveolin 1, caveolae protein, 22 kD | 2.93E−02 | 3.64 |
| 454985 | AW849292 | | gb: IL3-CT0215-020300-090-E06 CT0215 Homo | 2.04E−02 | 3.63 |
| 424554 | AA747563 | Hs. 131799 | ESTs, Weakly similar to ALU8_HUMAN ALU S | 2.93E−02 | 3.60 |
| 440535 | AI590563 | Hs. 125910 | ESTs | 5.43E−03 | 3.60 |
| 403180 | | | Target Exon | 1.15E−04 | 3.58 |
| 435715 | T78013 | Hs. 167279 | FYVE-finger-containing Rab5 effector pro | 7.62E−03 | 3.57 |
| 402651 | | | NM_000721*: Homo sapiens calcium channel, | 1.31E−03 | 3.57 |
| 419555 | AA244416 | | gb: nc07d11.s1 NCI_CGAP_Pr1 Homo sapiens | 1.53E−04 | 3.54 |
| 404359 | | | Target Exon | 5.67E−03 | 3.52 |
| 418525 | AW450369 | Hs. 86937 | ESTs | 2.32E−03 | 3.52 |
| 453899 | AW134536 | Hs. 243994 | ESTs | 5.20E−03 | 3.52 |
| 402889 | | | ENSP00000217123*: FLJ00118 protein (Fragm | 1.31E−03 | 3.52 |
| 403796 | | | Target Exon | 9.27E−04 | 3.50 |
| 404473 | | | ENSP00000247423: D-siglec precursor. | 1.18E−03 | 3.50 |
| 408120 | AW299900 | Hs. 267632 | TATA element modulatory factor 1 | 2.55E−03 | 3.50 |
| 447819 | U90544 | Hs. 19710 | solute carrier family 17 (sodium phospha | 2.45E−02 | 3.50 |
| 402023 | | | Target Exon | 7.83E−02 | 3.47 |
| 415897 | H08323 | Hs. 268712 | ESTs | 2.13E−04 | 3.47 |
| 445402 | AI222415 | Hs. 147852 | ESTs | 1.08E−03 | 3.47 |
| 404518 | | | CD83 antigen (activated B lymphocytes, i | 7.15E−02 | 3.45 |
| 450875 | AK000724 | | karyopherin alpha 6 (importin alpha 7) | 2.65E−04 | 3.45 |
| 454636 | AW811502 | | gb: QV2-ST0145-061299-015-b04 ST0145 Homo | 5.43E−03 | 3.44 |
| 403579 | | | Target Exon | 1.96E−02 | 3.40 |
| 456782 | AK000462 | Hs. 132071 | ovarian carcinoma immunoreactive antigen | 1.15E−04 | 3.39 |
| 433010 | AW970018 | | gb: EST382097 MAGE resequences, MAGK Homo | 3.05E−03 | 3.35 |
| 443725 | AW245680 | Hs. 9701 | growth arrest and DNA-damage-inducible, | 1.92E−05 | 3.33 |
| 400407 | AF210247 | Hs. 283949 | enamelin | 7.19E−04 | 3.32 |
| 432304 | AA932186 | Hs. 69297 | ESTs | 1.25E−03 | 3.32 |
| 447721 | BE619620 | | lysyl oxidase | 5.40E−05 | 3.32 |
| 433095 | AK001092 | Hs. 302480 | Homo sapiens cDNA FLJ10230 fis, clone HE | 2.92E−03 | 3.30 |
| 442364 | AA993149 | Hs. 129895 | ESTs, Moderately similar to TBX3_HUMAN T | 5.58E−04 | 3.30 |
| 444080 | AW812664 | | gb: RC4-ST0185-191099-012-h10 ST0185 Homo | 4.41E−02 | 3.30 |
| 403545 | | | Target Exon | 1.03E−03 | 3.26 |
| 454741 | BE154396 | | gb: CM2-HT0342-091299-050-b05 HT0342 Homo | 8.27E−03 | 3.24 |
| 428177 | AA423967 | | ESTs | 8.37E−04 | 3.24 |
| 454292 | N57559 | Hs. 82273 | hypothetical protein | 5.05E−02 | 3.22 |
| 427536 | BE277141 | Hs. 115803 | gb: 601178666F1 NIH_MGC_20 Homo sapiens c | 8.81E−04 | 3.20 |
| 439760 | AL355741 | Hs. 21641 | Homo sapiens mRNA full length insert cDN | 1.31E−03 | 3.20 |
| 450799 | AW407504 | | gb: UI-HF-BM0-adk-g-12-0-UI.r1 NIH_MGC_38 | 1.08E−03 | 3.16 |
| 447135 | T58148 | | gb: yb98g06.s1 Stratagene lung (937210) H | 2.38E−04 | 3.13 |
| 417079 | U65590 | Hs. 81134 | interleukin 1 receptor antagonist | 5.21E−03 | 3.11 |
| 428568 | AC004755 | | Homo sapiens chromosome 19, fosmid 37502 | 1.19E−03 | 3.11 |
| 406060 | | | Target Exon | 2.02E−03 | 3.07 |
| 421224 | AW402154 | Hs. 125812 | ESTs | 1.92E−03 | 3.05 |
| 459399 | BE407712 | Hs. 153998 | creatine kinase, mitochondrial 1 (ubiqui | 3.06E−03 | 3.05 |
| 440509 | BE410132 | Hs. 134202 | ESTs, Weakly similar to T17279 hypotheti | 2.25E−04 | 3.05 |
| 408952 | S79854 | Hs. 49322 | deiodinase, iodothyronine, type III | 6.52E−04 | 3.04 |
| 410619 | BE512730 | Hs. 65114 | keratin 18 | 8.62E−03 | 3.03 |
| 452236 | AI130858 | Hs. 143218 | ESTs | 1.29E−02 | 3.02 |
| 436651 | BE045962 | Hs. 275998 | ESTs | 5.55E−02 | 2.97 |
| 430071 | AA355986 | Hs. 232068 | transcription factor 8 (represses interl | 2.04E−02 | 2.97 |
| 405600 | | | C12001673: gi\|9631264\|ref\|NP_048045.1\|or | 2.19E−02 | 2.97 |
| 416666 | H73028 | Hs. 268992 | ESTs | 2.13E−04 | 2.96 |
| 414141 | BE255083 | Hs. 145729 | hypothetical protein DKFZp564A1164 | 2.92E−03 | 2.95 |
| 427408 | AA583206 | Hs. 2156 | RAR-related orphan receptor A | 9.92E−02 | 2.92 |
| 422017 | NM_003877 | Hs. 110776 | STAT induced STAT inhibitor-2 | 4.57E−03 | 2.92 |
| 444047 | AI097452 | | ESTs | 2.73E−02 | 2.90 |
| 440898 | AL035690 | Hs. 165 | glucagon-like peptide 1 receptor | 1.96E−02 | 2.90 |
| 408253 | AW807476 | Hs. 21051 | Homo sapiens mRNA for FLJ0012 protein, | 2.11E−02 | 2.88 |
| 446912 | AI347650 | Hs. 128521 | ESTs, Moderately similar to ALU4_HUMAN A | 2.54E−03 | 2.88 |
| 457148 | AF091035 | Hs. 184627 | KIAA0118 protein | 2.32E−03 | 2.86 |
| 444207 | AI565004 | | cathepsin D (lysosomal aspartyl protease | 3.53E−05 | 2.84 |
| 438460 | AB020702 | Hs. 6224 | KIAA0895 protein | 1.51E−03 | 2.82 |
| 405502 | | | C7000609*: gi\|628012\|pir\|\|A53933 myosin I | 4.72E−02 | 2.82 |
| 453560 | AA348626 | Hs. 5890 | hypothetical protein FLJ23306 | 1.19E−02 | 2.82 |
| 417213 | BE257508 | Hs. 24719 | modulator of apoptosis 1 | 5.05E−05 | 2.81 |
| 403582 | | | Target Exon | 8.57E−02 | 2.80 |
| 408112 | AW451982 | Hs. 248613 | ESTs | 2.83E−02 | 2.80 |
| 437180 | BE180234 | Hs. 281462 | Homo sapiens cDNA FLJ14793 fis clone NT | 2.67E−03 | 2.79 |
| 412429 | AV650262 | Hs. 75765 | GRO2 oncogene | 1.01E−06 | 2.79 |
| 421993 | R22497 | Hs. 110571 | growth arrest and DNA-damage-inducible, | 1.62E−04 | 2.79 |
| 407338 | AA773213 | Hs. 200558 | gb: ab66f10.s1 Stratagene lung carcinoma | 2.32E−03 | 2.78 |
| 448778 | AF074913 | | gb: Homo sapiens transcription factor Pax | 1.14E−02 | 2.77 |
| 422743 | BE304678 | Hs. 119598 | ribosomal protein L3 | 2.36E−02 | 2.75 |
| 427413 | BE547647 | Hs. 177781 | hypothetical protein MGC5618 | 5.43E−03 | 2.70 |
| 447306 | AI373163 | Hs. 170333 | ESTs | 5.21E−02 | 2.70 |
| 447526 | AL048753 | Hs. 303649 | small inducible cytokine A2 (monocyte ch | 2.20E−02 | 2.69 |
| 400818 | | | Target Exon | 2.26E−04 | 2.69 |
| 432128 | AA127221 | Hs. 296502 | ESTs | 3.25E−02 | 2.67 |
| 414890 | BE281095 | Hs. 77573 | uridine phosphorylase | 1.59E−03 | 2.65 |
| 413731 | BE243845 | Hs. 75511 | connective tissue growth factor | 2.45E−02 | 2.65 |
| 434180 | AA921757 | Hs. 116180 | ESTs | 1.37E−04 | 2.64 |
| 435992 | AI033259 | Hs. 118317 | Homo sapiens cDNA FLJ12088 fis, clone HE | 2.83E−02 | 2.63 |
| 419180 | T95449 | Hs. 220817 | ESTs | 1.56E−02 | 2.63 |
| 457054 | NM_014137 | Hs. 177258 | PRO0650 protein | 1.75E−02 | 2.63 |
| 432745 | AI821926 | | gb: nt78f05.x5 NCI_CGAP_Pr3 Homo sapiens | 7.21E−04 | 2.62 |
| 427899 | AA829286 | Hs. 332053 | serum amyloid A1 | 1.75E−03 | 2.61 |
| 405443 | | | Target Exon | 6.44E−03 | 2.61 |
| 428403 | AI393048 | Hs. 326159 | leucine rich repeat (in FLII) interactin | 6.94E−02 | 2.61 |
| 409463 | AI458165 | Hs. 17296 | hypothetical protein MGC2376 | 2.63E−02 | 2.60 |
| 402878 | | | ENSP00000217420*: BA122O1.1 (A novel prot | 3.12E−04 | 2.57 |
| 404046 | | | NM_019120*: Homo sapiens protocadherin be | 1.66E−03 | 2.56 |
| 458873 | AW150717 | Hs. 345728 | STAT induced STAT inhibitor 3 | 3.05E−03 | 2.55 |
| 426653 | AA530892 | Hs. 171695 | dual specificity phosphatase 1 | 2.52E−04 | 2.55 |
| 411920 | AW876263 | | gb: PM4-PT0019-131299-006-E09 PT0019 Homo | 1.75E−02 | 2.54 |
| 454339 | AW381980 | | gb: QV4-HT0316-091199-028-05 HT0316 Homo | 2.04E−02 | 2.52 |
| 432909 | AA570111 | | ESTs, Weakly similar to ALUE_HUMAN !!!! | 1.30E−03 | 2.51 |
| 411283 | AW852754 | | gb: PM1-CT0247-180100-009-c05 CT0247 Homo | 3.03E−02 | 2.50 |
| 415759 | AA169182 | Hs. 182740 | gb: zp20e02.s1 Stratagene fetal retina 93 | 2.43E−03 | 2.50 |
| 447650 | AW581199 | Hs. 161137 | ESTs, Moderately similar to 154374 gene | 8.28E−03 | 2.50 |
| 402012 | | | ubiquitin specific protease 15 | 3.88E−04 | 2.50 |
| 423499 | AW608884 | Hs. 28068 | ESTs | 2.96E−04 | 2.49 |
| 402124 | | | NM_031891: Homo sapiens cadherin 20, type | 3.05E−03 | 2.47 |
| 412755 | BE144306 | Hs. 179891 | ESTs, Weakly similar to P4HA_HUMAN PROLY | 1.38E−03 | 2.47 |
| 418443 | NM_005239 | Hs. 85146 | v-ets avian erythroblastosis virus E26 o | 1.37E−04 | 2.47 |
| 419239 | AA468183 | Hs. 184598 | Homo sapiens cDNA: FLJ23241 fis, clone C | 6.72E−02 | 2.47 |
| 419909 | AL136653 | Hs. 93675 | decidual protein induced by progesterone | 1.08E−03 | 2.45 |
| 402608 | | | CD83 antigen (activated B lymphocytes, i | 4.57E−02 | 2.44 |
| 402022 | | | Target Exon | 4.00E−03 | 2.42 |
| 458568 | AI769067 | Hs. 127824 | ESTs, Weakly similar to T28770 hypotheti | 8.65E−05 | 2.42 |
| 442702 | AW235697 | Hs. 130980 | ESTs | 6.12E−02 | 2.42 |
| 424236 | AW058114 | Hs. 7837 | phosphoprotein regulated by mitogenic pa | 6.17E−03 | 2.42 |
| 427700 | AA262294 | Hs. 180383 | dual specificity phosphatase 6 | 2.04E−02 | 2.41 |
| 410204 | AJ243425 | Hs. 326035 | early growth response 1 | 4.00E−03 | 2.41 |
| 424221 | NM_014478 | Hs. 300684 | calcitonin gene-related peptide-receptor | 8.59E−05 | 2.41 |
| 401376 | | | Target Exon | 8.26E−03 | 2.40 |
| 456147 | H41324 | Hs. 31581 | ESTs, Moderately similar to ST1B_HUMAN S | 4.28E−02 | 2.40 |
| 407654 | AW064121 | Hs. 279175 | ESTs | 1.83E−03 | 2.38 |
| 402364 | | | CCAAT/enhancer binding protein (C/EBP), | 3.25E−02 | 2.37 |
| 438808 | M73980 | Hs. 129053 | Homo sapiens NOTCH 1 (N1) mRNA, complete | 1.30E−05 | 2.36 |
| 401942 | | | C17001396*: gi\|3212355\|pdb\|1A4P\|A Chain A | 3.80E−03 | 2.33 |
| 426521 | AF161445 | Hs. 170219 | hypothetical protein | 3.03E−02 | 2.32 |
| 446378 | AI905699 | | citrate synthase | 3.18E−03 | 2.31 |
| 408935 | BE539706 | Hs. 285363 | ESTs | 4.77E−03 | 2.31 |
| 424028 | AF055084 | Hs. 153692 | Homo sapiens cDNA FLJ14354 fis, clone Y7 | 6.93E−02 | 2.29 |
| 433070 | N75346 | | CDC20 (cell division cycle 20, S. cerevi | 5.56E−02 | 2.29 |
| 456825 | H67220 | Hs. 169681 | death effector domain-containing | 1.24E−02 | 2.26 |
| 459711 | BE386801 | Hs. 21858 | trinucleotide repeat containing 3 | 8.29E−03 | 2.26 |
| 406140 | | | Target Exon | 8.12E−05 | 2.26 |
| 400327 | M18679 | | Human variant 5S rRNA-like gene and ORF, | 8.07E−02 | 2.25 |
| 455968 | BE168828 | | gb: QV1-HT0517-020400-145-f04 HT0517 Homo | 2.65E−04 | 2.25 |
| 431387 | AI878854 | Hs. 252229 | v-maf musculoaponeurotic fibrosarcoma (a | 1.75E−02 | 2.24 |
| 421233 | AA209534 | Hs. 284243 | tetraspan NET-6 protein | 1.82E−02 | 2.23 |
| 401522 | | | CGI-35 protein | 5.42E−03 | 2.22 |
| 422831 | R02504 | Hs. 332943 | ESTs | 1.14E−02 | 2.22 |
| 401346 | | | hypothetical protein | 2.93E−02 | 2.21 |
| 410268 | AA316181 | Hs. 61635 | six transmembrane epithelial antigen of | 2.36E−02 | 2.20 |
| 434495 | AW352170 | Hs. 129086 | Homo sapiens cDNA FLJ12007 fis, clone HE | 1.56E−02 | 2.20 |
| 442321 | AF207664 | Hs. 8230 | a disintegrin-like and metalloprotease ( | 1.69E−05 | 2.19 |
| 458692 | BE549905 | Hs. 231754 | ESTs | 7.60E−02 | 2.19 |
| 403750 | | | C5001092: gi\|6671939\|gb\|AAF23199.1\|AC0167 | 5.90E−03 | 2.18 |
| 441516 | F06700 | Hs. 7879 | interferon-related developmental regulat | 2.93E−02 | 2.18 |
| 423503 | M92843 | Hs. 343586 | zinc finger protein homologous to Zfp-36 | 1.19E−03 | 2.18 |
| 456465 | M94065 | Hs. 94925 | dihydroorotate dehydrogenase | 6.84E−05 | 2.17 |
| 440249 | AI246590 | Hs. 249175 | ESTs | 1.53E−04 | 2.16 |
| 459235 | BE246010 | | Homo sapiens mRNA for FLJ00038 protein, | 4.54E−04 | 2.15 |
| 409540 | AW409569 | Hs. 101550 | gb: fh01e09.x1 NIH_MGC_17 Homo sapiens cD | 7.98E−04 | 2.14 |
| 412541 | BE009398 | Hs. 74002 | nuclear receptor coactivalor 1 | 7.31E−03 | 2.13 |
| 449713 | AW027025 | | ESTs | 6.19E−04 | 2.13 |
| 442351 | W52642 | Hs. 8261 | hypothetical protein FLJ22393 | 8.39E−04 | 2.13 |
| 407869 | AI827976 | Hs. 24391 | hypothetical protein FLJ13612 | 3.06E−03 | 2.13 |
| 411372 | AI147861 | Hs. 213289 | low density lipoprotein receptor (famili | 7.71E−05 | 2.12 |
| 423168 | R34385 | Hs. 124940 | GTP-binding protein | 1.06E−02 | 2.11 |
| 414002 | NM_006732 | Hs. 75678 | FBJ murine osteosarcoma viral oncogene h | 4.02E−06 | 2.10 |
| 419048 | T91158 | Hs. 268605 | ESTs | 1.37E−03 | 2.09 |
| 433178 | AB038269 | Hs. 253706 | cysteinyl leukotriene CysLT2 receptor; c | 1.92E−03 | 2.08 |
| 432190 | T80206 | Hs. 14716 | ESTs | 1.25E−03 | 2.07 |
| 447261 | NM_006691 | Hs. 17917 | extracellular link domain-containing 1 | 9.36E−02 | 2.07 |
| 436869 | NM_014867 | Hs. 5333 | KIAA0711 gene product | 2.67E−03 | 2.06 |
| 402441 | | | Target Exon | 4.77E−03 | 2.05 |
| 441475 | AI929602 | Hs. 177 | phosphatidylinositol glycan, class H | 2.02E−04 | 2.05 |
| 404126 | | | ENSP00000211797*: Helicase SKI2W (Helicas | 1.08E−03 | 2.05 |
| 443514 | BE464288 | Hs. 141937 | ESTs | 8.57E−02 | 2.05 |
| 404187 | | | NM_019602: Homo sapiens butyrophilin-like | 3.35E−03 | 2.03 |
| 424125 | M31669 | Hs. 1735 | inhibin, beta B (activin AB beta polypep | 1.82E−02 | 2.03 |
| 400409 | AF153341 | | Homo sapiens winged helix/forkhead trans | 4.37E−03 | 2.02 |
| 404752 | | | NM_024778: Homo sapiens hypothetical prot | 2.82E−02 | 2.02 |
| 449338 | H73444 | Hs. 394 | adrenomedullin | 2.36E−02 | 2.02 |
| 437389 | AL359587 | Hs. 271586 | hypothetical protein DKFZp762M115 | 2.63E−02 | 2.02 |
| 401405 | | | Target Exon | 3.49E−02 | 2.01 |
| 429446 | AI547111 | | gb: PN2.1_A01_G12.r mynorm Homo sapiens c | 2.36E−02 | 2.01 |
| 431111 | AB033072 | Hs. 250015 | KIAA1246 protein | 1.44E−04 | 2.01 |
| 406210 | | | Target Exon | 1.82E−02 | 2.01 |
| 417076 | AW973454 | Hs. 115175 | ESTs, Moderately similar to ALU7_HUMAN A | 2.04E−02 | 2.00 |

Pkey: Unique Eos probeset identifier number
ExAccn: Exemplar Accession number, Genbank accession number
UnigeneID: Unigene number
Unigene Title: Unigene gene title
R1: Wilcoxon rank-sum test p-value
R2: Trimean of Hep C− Liver over Trimean of Hep C+ liver Ratio

TABLE 8B

| Pkey | CAT Number | Accession |
|---|---|---|
| 449986 | 2292_10 | AK055879 AW007836 AA873089 N74374 AV720071 AA702706 AW055276 |
| | | BE672779 AW864502 AI678780 AW864369 AI052145 T40984 |
| | | N74426 |
| 419299 | 36328_1 | BM477587 BE675426 AA827059 AI597639 AI571409 AI719948 AI311085 |
| | | AA953361 AI498787 AI364049 AI311084 BF871020 BE549868 |
| | | AI356384 AA236660 |
| 423053 | 367623_1 | BE314223 AA320990 BE312679 BI054000 BI324838 AW057717 |
| 408278 | MH337_68 | BG422281 BG422108 AW177973 BG750536 AW876813 AW876814 |
| 409795 | MH905_27 | AI498073 AI934808 AW374654 BI036066 BE709215 AA077985 BI831355 |
| | | BF932450 BF770705 BF379220 AA077682 AA076814 AA076875 |
| | | BI027970 AA077787 AA077311 BF811798 AW393752 BF327269 AA077305 |
| | | AA078114 BF800885 BF942930 AW903223 BF916052 AW903170 |
| | | AW903224 BF352664 BF379209 |
| 453196 | 505145_1 | AW003567 AI963955 AI990231 |
| 454598 | 1063474_1 | BF374577 AW809840 AW809996 AW809798 AW809695 AW809646 AW809738 BF374582 |
| | | AW809716 AW809826 AW809802 AW809747 |
| | | AW810152 |
| 412176 | 199671_1 | BF818635 AW898334 H66426 AW899792 |
| 458867 | 1246993_1 | AW995393 AJ403118 |
| 417732 | 299677_1 | R12062 R12616 Z43412 R36065 |
| 459476 | 169217_1 | AA584407 BE185844 BF764955 |
| 409840 | 915929_1 | AW502122 AW501663 AW501720 AW502125 |
| 454985 | 1087722_1 | AW849431 AW849428 AW849424 AW849422 AW849420 AW849292 AW849427 |
| 419555 | 252042_1 | AA244401 AA244416 |
| 450875 | 10801_1 | AL041364 BE393266 AA573189 BF589066 AI623423 AI889612 H54292 |
| | | AA085863 AA669816 BE542832 BI094274 AI360690 T61853 AW081194 |
| | | AI541147 AW750358 |
| 454636 | 1064998_2 | AW811471 AW811548 AW811521 |
| 433010 | 1234715_1 | AW970018 BE843649 BE843644 AA573622 R08736 AA573669 |
| 447721 | 1889_2 | BC013767 AL572931 BE742185 BI520113 AW959076 AI341487 AI623222 BM091074 |
| | | AW593800 AI983635 AW275114 AI952164 BM091378 |
| | | AA977038 AW513859 AI801910 AW273202 AW166266 AW337946 AI086791 |
| | | BE907359 AW273147 AI453134 AA250733 AW072844 AI818468 |
| | | AI561259 AW470887 AW300481 AW103087 AW175624 BE048584 BF063936 |
| | | AI207341 AW193240 AW193322 AW264492 AI682412 AI631778 |
| | | AI669677 AI128695 AA448630 AA456607 BF313680 BG898294 BI195544 |
| | | BG421755 BI760100 BE383304 BF329916 BF526599 AA385255 |
| | | BI520887 AA410939 AA448721 BF525380 BG423666 BI761786 BF944570 |
| 444080 | 20054_4 | AL545854 H05874 AW812663 BE146011 AW812664 AV647861 |
| | | AW812655 AW812611 BG718126 |
| 454741 | 220369_1 | BE154396 BF846839 AW842318 AW817959 BE154393 BI050168 |
| 428177 | 8737_7 | AA779866 AA423967 AA423968 |
| 450799 | 819959_1 | W31274 AW407504 AI738877 |
| 447135 | 579607_1 | T58148 AW516579 AW059603 |
| 428568 | 738084_1 | BE259137 BE251523 |
| 444047 | 56634_1 | AK057279 AI939345 BF748883 AI161300 AI097452 AI097450 BF958187 BE936529 |
| | | AW890840 AW890825 BE763814 AW408933 BI038453 |
| | | BE735308 |
| 444207 | 9172_3 | BE739425 AA514221 AA865491 AI828293 AA470456 AI276739 AA169357 BE932464 |
| | | AA514889 AW819039 AW819083 BE843048 AI432496 |
| | | AI470335 AI247243 BG533994 AA513783 AI887309 AA528036 AW972006 AW873028 AI924914 AI818810 |
| | | AW152378 AW084946 AI521413 |
| | | AI669583 BE932521 AI581370 BE180238 AW089750 AW771461 AW089714 AI590949 AI819148 |
| | | AA731056 BF815234 BF911506 AA235803 |
| | | AA485373 AI735658 AW393133 AW073080 AI707637 BF353320 BE843111 AW819036 AW393135 |
| | | BG697291 AV648670 AV654332 AV687530 |
| | | BG566964 AI807430 AI676072 AA837010 AI452482 AI625817 AW241750 BE048616 AI290928 |
| | | AI680714 AA485530 BE175687 AV648513 |
| | | AW130312 AI000556 AA632893 BE674169 BF001208 AA948166 BE175650 AA524664 AA490345 |
| | | AI244948 AA602956 AA483492 AA918178 |
| | | AW802049 BG675859 AV658871 BG678060 AI565004 AW819026 BE843092 AV686437 AV723049 |
| | | BG616948 AI911647 AI743490 AI091096 |
| | | BE857251 AI962074 AA040027 AW769317 AA343477 AA640112 BF876213 R82948 H26425 H82876 |
| | | BE843095 BE843140 BG536641 |
| | | BG617830 AA235802 BE774985 BE006682 BF342375 AA903144 BF338083 BF984258 AV657996 |
| | | AI749532 BE768614 BE857252 BE932516 |
| | | BE768573 AV657993 AV657777 AV752631 BE774974 T55847 BF095761 BF911511 BE710793 |
| | | BE180119 BG617338 H45942 T55897 AV657718 |
| | | BG563497 |
| 448778 | 7800_1 | AW505435 U62539 |
| 432745 | 112643_1 | AA658826 AI821926 AI791191 AA635129 AA564492 |
| 411920 | 1141803_1 | AW876134 AW876141 AW876398 AW876328 AW876308 AW876331 AW876415 AW876326 |
| | | AW876181 AW876138 AW876131 AW876240 |
| | | AW876206 AW876289 AW876218 AW876261 AW876343 AW876347 AW876188 |
| | | AW876371 AW876273 AW876231 AW876191 AW876209 |
| | | AW876322 AW876314 AW876165 AW876363 AW876378 AW876376 AW876169 AW876412 AW876426 |
| | | AW876407 AW876257 AW876263 |
| | | AW876366 AW876334 |
| 454339 | 789006_1 | BE152238 AW381980 BE152235 BE152244 BE152232 |
| 432909 | 137712_1 | AA702596 AA570111 AI348435 AW192161 |
| 411283 | 215263_1 | AW852754 AW852757 AW852617 BE172755 AW852897 AW835444 AW835440 |
| | | AW801490 AW801489 BF839901 AW835520 |
| 446378 | 10288_1 | BC010106 AL560552 AU133296 AU133086 BE268567 BE268523 BI544879 |
| | | BE398161 BG473088 BI544445 BE258021 BE296339 BE255040 |
| | | BE263020 BG706790 AL598627 AW952337 BG758113 AW512753 BE267666 BE253415 BI225718 BE268350 |
| | | BE258245 BI224965 AW772605 |
| | | BG723903 BE397282 BI196341 BG702880 AI878949 AL600437 AA416759 BE259917 AI031582 BF512142 |
| | | AI088248 BE560328 AI802623 |
| | | AI288613 AL597585 AW768553 AI816352 BF732831 BI225687 AA833686 AA722593 AA807750 |
| | | AW068064 AA405187 AI923236 N51593 |
| | | AL527710 BG282576 AL525927 AL525971 BI869547 AI064725 R91856 H46814 H20112 |
| | | W01682 AW848870 AW848585 AW376662 AW848985 |
| | | AW848937 AW848862 AW848581 AW848512 AW848176 AW752623 AW752618 AW376822 |
| | | AW376821 AW376684 AW376623 AW376622 |
| | | BE706047 AW752602 AW752691 AW752674 AW752652 AA379167 AW752610 AW752684 |
| | | AW752613 AW752660 AW848709 AW848576 |
| | | AW849155 AW848981 AW848980 AW848979 AW848978 AW848973 AW848916 AW848713 |
| | | AW848708 AW848642 AW848641 AW848639 |
| | | AW848573 AW848493 AW848492 AW848489 AW848488 AW848487 AW848353 AW848352 |
| | | AW848220 AW752698 AW752697 AW752682 |
| | | AW752681 AW752680 AW752679 AW752664 AW752651 AW752638 AW752637 AW752636 |
| | | AW752628 AW752626 AW752624 AW752619 |
| | | AW752596 AW752608 AL582019 BE875587 AL529175 AW965868 BG686208 AA259073 |
| | | BE696973 AA459543 AA358314 W40564 BF926427 |
| | | AW849000 AW848718 AW848515 AW848507 AW848444 AW848440 AW848232 AW848222 |
| | | AW752657 AW376786 AW376781 AW376615 |
| | | AW376614 BI752581 AA534520 AI748906 AA047799 AI014753 AL514460 AL581982 |
| | | BG743146 W24171 H20102 H11227 AW752607 AW006596 |
| | | AW130378 BE716519 AW752661 AW848289 BF349557 AW752612 AW752632 AW848910 |
| | | NM_004077 AF047042 AL560606 BI765896 |
| | | BI196831 BI855656 BE906674 BG749937 BE535486 BE019810 AA313713 AA992542 |
| | | AA332541 AA682985 AA356125 BE140478 BG750945 |
| | | BI457548 BG025661 BF326302 AA325019 BG980676 AA337465 AA321974 BG949285 |
| | | BG427585 R23979 BG611485 BE560678 W16977 |
| | | N50379 BG824101 BG471750 BI463171 W04691 AU099360 BG471590 |
| | | BM011999 BE262945 BE559801 BF756438 BE881957 BE314546 |
| | | BG911831 BG150811 BG112017 AA157518 T92368 AW752620 |
| 433070 | 22399_1 | BE794397 BG121933 BI194378 AW410585 AW954321 BI045952 AW024741 |
| | | AW444579 AI973044 AI075432 AI093319 AI635673 AA625246 |
| | | D45465 BE173394 AV724875 AW954889 AI366776 AI498872 AA027096 AI351434 AA916072 |
| | | AA302868 AA535890 AI420076 AI669179 |
| | | AI240010 AI201405 AW451691 AA317478 AA424952 AW772292 BE857671 |
| | | AI869583 AI470411 AI804946 AI744155 D80532 |
| 455968 | 1557068_1 | BE168828 BE168823 BE168830 BE168820 BE168931 BE168826 BE168928 |
| 459235 | 26808_3 | AW814516 AW815927 AW814504 BF375203 AW814522 AW814521 |
| | | AW814524 BF375206 AW814518 AW814517 AI940652 BF837881 |
| | | AW751232 BF374342 AW176453 AW814505 BF886651 |
| | | AW814525 BG001382 BE933380 |
| 449713 | 519163_1 | AW027025 AW028264 AI660390 |
| 429446 | 352_30 | AI547111 BG945630 BG913104 AA558007 AW973749 |

Pkey: Unique Eos probeset identifier number
CAT number: Gene cluster number
Accession: Genbank accession numbers

TABLE 8C

| Pkey | Ref | Strand | Nt_position |
|---|---|---|---|
| 405278 | 6139075 | Minus | 3863-3965, 4823-4891, |
| | | | 5439-5529, 6043-6170 |
| 404501 | 7229859 | Minus | 37270-37526 |
| 401149 | 7229925 | Plus | 73117-74019 |
| 402273 | 2979528 | Plus | 28990-29203, 32299-32402, |
| | | | 32474-32668 |
| 402517 | 9798106 | Plus | 17569-17721 |
| 401553 | 8099284 | Minus | 83990-84161 |
| 404958 | 7407941 | Minus | 2731-4531 |
| 403324 | 8440025 | Minus | 107104-107309 |
| 401913 | 9369520 | Minus | 33753-33904 |
| 403794 | 8096910 | Plus | 163292-163884 |
| 403180 | 7523976 | Minus | 63603-63759 |
| 402651 | 7960391 | Plus | 174215-174380 |
| 404359 | 7630876 | Minus | 11789-12516 |
| 402889 | 9931133 | Plus | 89392-89498, 90358-90571 |
| 403796 | 8099896 | Minus | 75073-77664 |
| 404473 | 8079921 | Plus | 22639-22773 |
| 402023 | 7528158 | Minus | 132872-133040 |
| 404518 | 8151988 | Plus | 84494-84603 |
| 403579 | 8101179 | Minus | 36167-36365 |
| 403545 | 8078400 | Plus | 25293-25640 |
| 406060 | 6899623 | Minus | 20339-20746 |
| 405600 | 5923640 | Plus | 26662-27225 |
| 405502 | 9211311 | Minus | 50360-50584 |
| 403582 | 8101186 | Plus | 18308-18458 |
| 400818 | 8569994 | Plus | 172644-172765, 173085-173200 |
| 405443 | 7408143 | Plus | 90716-90887, 101420-101577 |
| 402878 | 9908870 | Minus | 56133-56522 |
| 404046 | 3688074 | Minus | 2-773 |
| 402012 | 7407997 | Minus | 111771-111909, 112107-112226, |
| | | | 112519-11269 |
| 402124 | 4033680 | Plus | 164206-164459 |
| 402608 | 9910096 | Plus | 37495-37669 |
| 402022 | 7139714 | Plus | 165595-165748 |
| 401376 | 7417809 | Plus | 40584-40963 |
| 402364 | 9454515 | Minus | 54983-55240, 56507-56785, |
| | | | 56982-57365 |
| 401942 | 4982556 | Minus | 130749-131044 |
| 406140 | 9168231 | Minus | 49887-50219 |
| 401522 | 7717114 | Minus | 154437-154847, 155420-155505, |
| | | | 155568-15615 |
| 401346 | 9926605 | Minus | 12031-13032 |
| 403750 | 7229814 | Minus | 133638-134110 |
| 402441 | 9796503 | Plus | 140903-141106 |
| 404126 | 9796876 | Plus | 48919-49155 |
| 404187 | 4481839 | Plus | 7644-7991 |
| 404752 | 7109522 | Minus | 120168-120326 |
| 401405 | 7768126 | Minus | 69276-69452, 69548-69958 |
| 406210 | 7341959 | Plus | 58546-58687 |

Pkey: Unique number corresponding to an Eos probeset
Ref: Sequence source. The 7 digit numbers in this column are Genbank Identifier (GI) numbers. “Dunham, et al.” refers to the publication entitled “The DNA sequence of human chromosome 22” Dunham, et al. (1999) Nature 402: 489-495.
Strand: Indicates DNA strand from which exons were predicted.
Nt_position: Indicates nucleotide positions of predicted exons.

Table 9A lists about 100 genes up-regulated in Hepatitis C positive liver tissues from patients that are non-responsive to pegylated-interferon-alpha plus ribavirin treatment (non-responders) compared to Hepatitis C positive liver tissues from patients that are responsive to the treatment (responders). In both cases, the liver biopsies were obtained prior to treatment. The 100 genes have the potential to be diagnostics and/or prognostic markers for determining the responsiveness of Hepatitis C infected liver tissues to pegylated-interferon-alpha plus ribavirin treatment. They may also provide clinical information on Hepatitis C infection and pathology. They may also be potential targets for therapeutic drugs and/or treatments. These were selected from 59680 probesets on the Affymetrix/Eos Hu03 GeneChip array such that the Wilcoxon rank-sum test p-value between the 2 groups was less than 0.10, the ratio of the “weighted average” of non-responders to the “weighted average” of responders was equal to or above 2.0, and that the differences between the same 2 groups was equal to or above 30.0. The “weighted average” of the non-responders was set to the trimean of several different non-responders. The “weighted average” of the responders was set to the either 10 or the trimean of several different responders, whichever value was greater to eliminate ratios with a denominator of zero or less.

TABLE 9A

| Pkey | ExAccn | UniGene | UnigeneID | R1 | R2 |
|---|---|---|---|---|---|
| 444342 | NM_014398 | Hs. 10887 | similar to lysosome-associated membrane | 2.34E−02 | 5.21 |
| 431326 | AW970580 | Hs. 198689 | KIAA0728 protein | 2.34E−02 | 4.92 |
| 424017 | AA333789 | | gb: EST37925 Embryo, 9 week Homo sapiens | 5.29E−02 | 4.82 |
| 410099 | AA081630 | | KIAA0036 gene product | 1.13E−02 | 4.77 |
| 407360 | X13075 | | gb: Human 2a12 mRNA for kappa-immunoglobu | 6.19E−02 | 4.52 |
| 447514 | AI809314 | Hs. 208501 | ESTs, Weakly similar to B34087 hypotheti | 8.30E−02 | 4.52 |
| 437233 | D81448 | Hs. 339352 | Homo sapiens brother of CDO (BOC) mRNA, | 1.34E−02 | 4.47 |
| 454525 | BE280421 | Hs. 94499 | ESTs | 4.53E−02 | 4.47 |
| 452455 | N25153 | | ESTs, Weakly similar to AF174605 1 F-box | 2.31E−02 | 4.45 |
| 458393 | AI792868 | Hs. 135365 | ESTs | 8.24E−02 | 4.30 |
| 458026 | H12028 | Hs. 6396 | jumping translocation breakpoint | 1.63E−02 | 4.20 |
| 459171 | AW967801 | Hs. 151293 | ESTs, Weakly similar to T42705 hypotheti | 4.55E−02 | 4.17 |
| 426798 | AA385062 | Hs. 130260 | ESTs | 8.27E−02 | 4.13 |
| 447339 | AI770001 | Hs. 209445 | ESTs | 4.55E−02 | 4.10 |
| 447686 | AI939440 | | ESTs | 9.52E−02 | 4.10 |
| 433312 | AI241331 | Hs. 131765 | ESTs, Moderately similar to I38937 DNA/R | 3.29E−02 | 4.09 |
| 435391 | AA704588 | Hs. 58934 | ESTs | 5.29E−02 | 4.05 |
| 437994 | U92012 | Hs. 251659 | ESTs, Weakly similar to mariner transpos | 3.83E−02 | 4.00 |
| 437990 | AI686579 | Hs. 121784 | ESTs | 7.15E−02 | 3.97 |
| 406355 | | | C5000598: gi\|2136258\|pir\|\|I59377 template | 8.30E−02 | 3.95 |
| 401745 | | | Target Exon | 2.34E−02 | 3.92 |
| 435903 | AA701890 | Hs. 118163 | ESTs | 4.43E−02 | 3.92 |
| 407366 | AF026942 | Hs. 17518 | gb: Homo sapiens cig33 mRNA, partial sequ | 1.64E−02 | 3.80 |
| 442269 | AI797066 | Hs. 201995 | ESTs | 1.10E−02 | 3.75 |
| 452941 | AL110347 | Hs. 31074 | N-sulfoglucosamine sulfohydrolase (sulfa | 5.11E−03 | 3.72 |
| 455604 | BE011183 | | gb: PM3-BN0218-100500-003-d09 BN0218 Homo | 4.55E−02 | 3.58 |
| 401376 | | | Target Exon | 8.27E−02 | 3.55 |
| 456445 | AK000735 | Hs. 93581 | hypothetical protein FLJ10512 | 3.86E−02 | 3.52 |
| 416814 | AW192307 | Hs. 80042 | dolichyl-P-Glc: Man9GlcNAc2-PP-dolichylgl | 8.30E−02 | 3.50 |
| 444640 | AL133933 | Hs. 64310 | interleukin 11 receptor, alpha | 3.27E−02 | 3.48 |
| 445882 | AI948717 | Hs. 225155 | ESTs, Weakly similar to A46302 PTB-assoc | 6.17E−02 | 3.42 |
| 453471 | AL037887 | Hs. 208179 | ESTs | 8.30E−02 | 3.42 |
| 449756 | W16700 | Hs. 94542 | ESTs | 8.27E−02 | 3.42 |
| 437774 | AW978199 | Hs. 291648 | ESTs, Weakly similar to I38022 hypotheti | 6.17E−02 | 3.30 |
| 437929 | T09353 | Hs. 106642 | ESTs, Weakly similar to T09052 hypotheti | 6.19E−02 | 3.30 |
| 405513 | | | ENSP00000241075: TRRAP PROTEIN. | 7.15E−02 | 3.28 |
| 404098 | | | Target Exon | 8.27E−02 | 3.25 |
| 408570 | AL046406 | Hs. 103483 | KIAA1798 protein | 7.12E−02 | 3.22 |
| 426101 | AL049987 | | Homo sapiens mRNA; cDNA DKFZp564F 112 (fr | 4.55E−02 | 3.22 |
| 447164 | AF026941 | Hs. 17518 | vipirin; similar to inflammatory respon | 8.30E−02 | 3.20 |
| 401242 | | | mitogen-activated protein kinase 8 inter | 4.55E−02 | 3.10 |
| 435154 | AA668764 | | ESTs | 8.30E−02 | 3.10 |
| 449137 | AW134478 | Hs. 196033 | ESTs | 3.32E−03 | 3.05 |
| 448497 | BE613269 | Hs. 21893 | hypothetical protein DKFZp761N0624 | 2.31E−02 | 3.02 |
| 410541 | AA065003 | Hs. 64179 | syntenin-2 protein | 8.30E−02 | 3.02 |
| 405723 | | | Target Exon | 8.30E−02 | 3.00 |
| 449613 | N63808 | Hs. 34299 | ESTs | 2.76E−02 | 3.00 |
| 459697 | AA406062 | Hs. 98002 | ESTs | 7.59E−03 | 3.00 |
| 417958 | AA767382 | Hs. 193417 | ESTs | 8.27E−02 | 2.97 |
| 444736 | AA533491 | Hs. 23317 | hypothetical protein FLJ14681 | 9.49E−02 | 2.96 |
| 417042 | C75563 | Hs. 113029 | ribosomal protein S25 | 6.14E−02 | 2.92 |
| 403512 | | | C3000579*: gi\|12643308\|sp\|Q9Y4K1\|AIM1_HUM | 7.66E−03 | 2.92 |
| 404226 | | | Target Exon | 4.53E−02 | 2.90 |
| 443884 | N20617 | Hs. 194397 | leptin receptor | 8.30E−02 | 2.88 |
| 432222 | AI204995 | | gb: an03c03.x1 Stratagene schizo brain S1 | 8.30E−02 | 2.88 |
| 407284 | AI539227 | Hs. 214039 | hypothetical protein FLJ23556 | 6.19E−02 | 2.86 |
| 439737 | AI751438 | Hs. 41271 | Homo sapiens mRNA full length insert cDN | 2.34E−02 | 2.83 |
| 408503 | AW119059 | | ESTs, Weakly similar to T12552 hypotheti | 8.30E−02 | 2.80 |
| 419743 | AW408762 | Hs. 5957 | Homo sapiens clone 24416 mRNA sequence | 8.30E−02 | 2.77 |
| 456727 | BE350986 | Hs. 33254 | hypothetical protein FLJ21817 similar to | 1.11E−02 | 2.75 |
| 440409 | AW294316 | Hs. 125608 | ESTs | 3.27E−02 | 2.73 |
| 408891 | NM_006577 | Hs. 284284 | ESTs, Highly similar to beta-1,3-N-acety | 4.53E−02 | 2.70 |
| 430291 | AV660345 | Hs. 238126 | CGI-49 protein | 2.34E−02 | 2.66 |
| 410851 | AW612147 | Hs. 32058 | Homo sapiens C1orf19 mRNA, partial cds | 7.15E−02 | 2.65 |
| 414737 | AI160386 | Hs. 125087 | ESTs | 3.86E−02 | 2.65 |
| 422744 | AW268803 | Hs. 119640 | hBKLF for basic kruppel like factor | 5.24E−02 | 2.60 |
| 429867 | AW204372 | | tomosyn | 5.19E−02 | 2.56 |
| 431926 | AW972724 | | gb: EST384816 MAGE resequences, MAGL Homo | 5.29E−02 | 2.55 |
| 430259 | BE550182 | Hs. 127826 | RalGEF-like protein 3, mouse homolog | 3.35E−03 | 2.54 |
| 428558 | AI587502 | | ESTs | 9.52E−02 | 2.53 |
| 435145 | AI277259 | Hs. 116631 | ESTs | 2.76E−02 | 2.52 |
| 403436 | | | NM_006548*: Homo sapiens IGF-II mRNA-bind | 8.30E−02 | 2.52 |
| 447698 | AI420156 | Hs. 326733 | ESTs | 7.15E−02 | 2.52 |
| 407339 | AA777542 | Hs. 132670 | ESTs | 2.76E−02 | 2.50 |
| 410401 | AW673335 | Hs. 259641 | ESTs | 2.34E−02 | 2.50 |
| 418481 | M81945 | | CD34 antigen | 3.29E−02 | 2.50 |
| 430654 | AW970081 | Hs. 325603 | ESTs | 8.27E−02 | 2.50 |
| 433102 | AI343966 | Hs. 158528 | ESTs | 1.13E−02 | 2.44 |
| 442191 | W95186 | Hs. 8136 | endothelial PAS domain protein 1 | 3.86E−02 | 2.42 |
| 447384 | AI377221 | Hs. 40528 | ESTs | 7.15E−02 | 2.40 |
| 430552 | AA176374 | Hs. 243886 | nuclear autoantigenic sperm protein (his | 6.14E−02 | 2.38 |
| 444811 | AW137791 | Hs. 148419 | ESTs | 1.64E−02 | 2.37 |
| 435519 | AI218950 | Hs. 125461 | hypothetical protein FLJ11539 | 5.29E−02 | 2.37 |
| 435675 | AA694099 | Hs. 266820 | ESTs | 8.30E−02 | 2.36 |
| 402305 | | | C19000735*: gi\|4508027\|ref\|NP_003414.1\|z | 1.12E−02 | 2.35 |
| 438084 | AW516099 | Hs. 121925 | ESTs | 8.30E−02 | 2.32 |
| 450747 | AI064821 | Hs. 318535 | ESTs, Highly similar to 1818357A EWS gen | 7.15E−02 | 2.32 |
| 452032 | BE244005 | Hs. 27610 | retinoic acid-and interferon-inducible | 8.27E−02 | 2.25 |
| 455751 | BE075281 | | gb: PM1-BT0585-290200-005-d07 BT0585 Homo | 8.10E−02 | 2.21 |
| 427471 | AA403131 | Hs. 266782 | KIAA1826 protein | 8.30E−02 | 2.20 |
| 437210 | AA311443 | Hs. 293563 | Homo sapiens mRNA; cDNA DKFZp586E2317 (f | 6.19E−02 | 2.17 |
| 458025 | AI275406 | Hs. 32450 | gb: ql63c10.x1 Soares_NhHMPu_S1 Homo sapi | 2.76E−02 | 2.17 |
| 444830 | AI198854 | Hs. 145437 | ESTs | 6.17E−02 | 2.12 |
| 455086 | AW855386 | | gb: CM3-CT0275-191099-024-d05 CT0275 Homo | 8.30E−02 | 2.10 |
| 421904 | BE143533 | Hs. 109309 | hypothetical protein FLJ20035 | 4.55E−02 | 2.07 |
| 414395 | BE304888 | Hs. 279834 | EST | 3.27E−02 | 2.02 |
| 404973 | | | Target Exon | 8.27E−02 | 2.01 |
| 428701 | NM_013276 | Hs. 190207 | carbohydrate kinase-like | 3.86E−02 | 2.01 |
| 436651 | BE045962 | Hs. 275998 | ESTs | 1.34E−02 | 2.00 |
| 448959 | AI610343 | Hs. 293267 | ESTs | 7.12E−02 | 2.00 |

Pkey: Unique Eos probeset identifier number
ExAccn: Exemplar Accession number, Genbank accession number
UnigeneID: Unigene number
Unigene Title: Unigene gene title
R1: Wilcoxon rank-sum test p-value
R2: Trimean of non-responders over Trimean of responders Ratio

TABLE 9B

| Pkey | CAT Number | Accession |
|---|---|---|
| 424017 | 888651_1 | AW966107 AA334317 AA333789 |
| 410099 | 16732_1 | AK055674 AW965247 AV751598 AA290926 R53043 AA331387 AK056148 BI917678 |
| | | BG819395 BG911971 BG820167 AI174254 AA348720 |
| | | AA364503 BG714279 AW893230 AA081774 H24222 AV727176 BF875715 |
| | | AA081630 BE000834 AA334880 AL563737 BG029709 W52882 |
| | | AI439658 BE551237 AA283724 BF109530 AI457096 AI805992 BE467736 AA693467 |
| | | AI697593 AI887863 AI167419 AW901980 AW901768 |
| | | BE702179 AA484549 T23811 BE327043 AA716027 AA917004 AA167714 BF339675 |
| | | AA084618 AI418634 T31586 AA436630 AI366472 |
| | | AA706191 AI422304 AI204899 AI041169 AA211402 AW827081 AA788593 T32736 |
| | | AI767935 AA747914 T03534 AW959843 AL119527 |
| | | BE327037 AW901982 AW993370 AW901977 AW902071 W60090 N79906 D52685 |
| | | T07735 BE702069 BE702172 T08671 BE767121 BE767117 |
| | | BE767113 |
| 452455 | 928588_1 | BE877195 BG988071 AI902576 N25153 AW518714 N25154 |
| 447686 | 2958543_1 | AI939440 AI424128 AI419682 |
| 455604 | 1478710_1 | BE011183 BE011324 BE011170 BE011188 BE011181 BE011333 BE011169 BE011161 |
| 426101 | 3211_1 | AL049987 BG620667 BG571984 AW362842 BE150456 BE326465 |
| | | AW872412 AA868553 AI024689 AA442638 AA813604 AA442648 AA663108 |
| | | AA442379 AA229448 N56349 AA460220 AW971193 AA453725 AI742087 |
| | | AI860142 AW769479 AI917507 AI860141 BE045272 AW277065 |
| | | AI921333 AI354470 BE466760 AI827987 AI005467 AA833517 AA563934 |
| | | AA522837 AA812876 AW020895 AA600372 AA663178 AI187977 |
| | | AA229164 AW270324 AA703066 T78981 AA632986 BE708493 R31132 AI253986 |
| | | AI916737 T84796 T84294 AW961515 AI459289 BF109829 |
| | | BI491853 AI084517 AW103830 BE835233 A1472712 AV741009 AA551512 |
| | | N28268 AA436880 AA447794 BE835410 BE835385 BE818352 |
| | | BE818350 R64648 BE646467 AA493776 AA437299 BE818343 R95914 R31089 |
| | | BF576826 AU186065 BF802058 AI217018 AA247541 AI191725 |
| | | BE766918 |
| 435154 | 126605_1 | AW972063 AA668764 AA804491 AW665688 AA765069 |
| 432222 | 539529_1 | BG207209 BE166299 AI204995 BG199355 AW969908 AA528756 AW440776 BI044354 |
| 408503 | 24663_2 | NM_054110 BC014789 BI552129 BG896227 BG900557 AY035399 AW119059 |
| | | BF432376 AW629242 AA971319 AI378176 AI275884 AA236602 |
| | | BG898257 BG897636 BG900588 AF361251 BG926353 BG714178 |
| | | AA368180 BG896473 BG715977 BG900230 BG901163 BF342939 |
| 429867 | 1003_2 | BE467551 AI214965 AW204372 |
| 431926 | 1237041_1 | AW972724 AA877998 AA522631 AU185388 |
| 428558 | 1382530_1 | AA430150 AA888768 AI587502 |
| 418481 | 17381_1 | S53911 NM_001773 AL572644 AL550179 AI688653 AW025002 AW614285 |
| | | AU158779 AI017002 AA434387 AI252665 AI262206 AU147582 |
| | | AI144193 AW952860 AI128776 AI017793 AI160509 AA906021 AI149563 |
| | | AU154950 AI128488 W74409 AI970362 AI141453 AU158772 W58493 |
| | | BM148338 R73091 AW020496 BI491517 AA022917 AI432610 AI879448 |
| | | R69099 AI708954 AW014274 AA483672 AA528783 AA912271 |
| | | AI926942 BE677587 AI874217 AA152376 AA640408 AI287334 BF830285 |
| | | AA311473 C18678 AA922603 C00910 AI124073 R38730 AA043439 |
| | | W94644 W58646 AA664247 BE061934 H01096 R69613 AI383162 AU133723 |
| | | AA311526 R67942 H01097 H72113 R72430 R39494 AV744074 |
| | | AA535925 BI759288 BI052385 BF854687 AW608286 AA043438 R72478 |
| | | AL513811 R69214 AA188435 AA054965 |
| 455751 | 1497052_1 | BE075281 BE075119 BE075123 BE075219 BE075046 |
| 455086 | 1095746_1 | AW855553 AW855417 AW855386 |

Pkey: Unique Eos probeset identifier number
CAT number: Gene cluster number
Accession: Genbank accession numbers

Table 10A lists about 149 genes up-regulated in Hepatitis C positive liver tissues from patients that are responsive to pegylated-interferon-alpha plus ribavirin treatment (responders) compared to Hepatitis C positive liver tissues from patients that are not responsive to the treatment (non-responders). In both cases, the liver biopsies were obtained prior to treatment. The 149 genes have the potential to be diagnostics and/or prognostic markers for determining the responsiveness of Hepatitis C infected liver tissues to pegylated-interferon-alpha plus ribavirin treatment. They may also provide clinical information on Hepatitis C infection and pathology. They may also be potential targets for therapeutic drugs and/or treatments. These were selected from 59680 probesets on the Affymetrix/Eos Hu03 GeneChip array such that the Wilcoxon rank-sum test p-value between the 2 groups was less than 0.10, the ratio of the “weighted average” of responders to the “weighted average” of non-responders was equal to or above 2.0, and that the differences between the same 2 groups was equal to or above 30.0. The “weighted average” of the responders was set to the trimean of several different responders. The “weighted average” of the non-responders was set to the either 10 or the trimean of several different non-responders, whichever value was greater to eliminate ratios with a denominator of zero or less.

TABLE 10A

| Pkey | ExAccn | UniGene | Unigene Title | R1 | R2 |
|---|---|---|---|---|---|
| 400301 | X03635 | Hs. 1657 | estrogen receptor 1 | 5.11E−03 | 8.42 |
| 440700 | AW952281 | Hs. 296184 | guanine nucleotide binding protein (G pr | 1.63E−02 | 6.41 |
| 421911 | AL041520 | | gb: DKFZp434G2317_s1 434 (synonym: htes3) | 3.27E−02 | 5.67 |
| 400302 | N48056 | Hs. 283946 | folate hydrolase (prostate-specific memb | 2.34E−02 | 5.61 |
| 414559 | AV656184 | Hs. 76452 | C-reactive protein, pentraxin-related | 3.86E−02 | 5.60 |
| 418363 | AA218628 | Hs. 202977 | ESTs | 8.30E−02 | 5.50 |
| 427450 | AB014526 | Hs. 178121 | KIAA0626 gene product | 1.63E−02 | 5.29 |
| 404354 | | | Target Exon | 1.64E−02 | 5.05 |
| 436674 | AA725002 | Hs. 272018 | low molecular mass ubiquinone-binding pr | 8.30E−02 | 5.00 |
| 455191 | AW864547 | | gb: PM4-SN0016-120500-003-b12 SN0016 Homo | 6.17E−02 | 4.90 |
| 415374 | F06904 | Hs. 8346 | ESTs | 6.19E−02 | 4.83 |
| 404483 | | | C8000657*: gi\|1504040\|dbj\|BAA13219.1\|(D8 | 1.13E−02 | 4.77 |
| 444813 | AW054834 | Hs. 210356 | ESTs | 8.30E−02 | 4.77 |
| 401197 | | | ENSP00000229263*: HSPC213. | 8.27E−02 | 4.75 |
| 449959 | AI076834 | Hs. 21160 | ESTs | 2.76E−02 | 4.60 |
| 444402 | AL138436 | Hs. 49359 | hypothetical protein DKFZp547E052 | 2.30E−02 | 4.57 |
| 451898 | T92572 | Hs. 142019 | ESTs, Weakly similar to 1207289A reverse | 1.64E−02 | 4.55 |
| 457231 | AI472022 | Hs. 301959 | praline synthetase co-transcribed (bacte | 4.55E−02 | 4.52 |
| 413303 | AW836130 | Hs. 75277 | hypothetical protein FLJ3910 | 8.20E−02 | 4.40 |
| 435147 | AL133731 | Hs. 4774 | Homo sapiens mRNA; cDNA DKFZp761C1712 (f | 6.19E−02 | 4.40 |
| 442552 | R20624 | | son of sevenless (Drosophila) homolog 1 | 6.19E−02 | 4.40 |
| 401635 | | | C11000702: gi\|10048448\|ref\|NP_065258.1\|g | 9.52E−02 | 4.27 |
| 430048 | T65054 | Hs. 73605 | ESTs | 8.27E−02 | 4.27 |
| 406570 | | | NM_024889*: Homo sapiens hypothetical pro | 2.34E−02 | 4.17 |
| 433122 | AB019391 | Hs. 58049 | ESTs | 7.15E−02 | 4.13 |
| 431940 | H06075 | Hs. 270232 | ESTs | 3.35E−03 | 4.07 |
| 411047 | AW938479 | | gb: CM0-DT0057-290200-253-h06 DT0057 Homo | 8.27E−02 | 4.04 |
| 403609 | | | C3001199: gi\|7494834\|pir\|\|T15308 hypothet | 3.29E−02 | 4.00 |
| 430165 | AW849764 | Hs. 314330 | ESTs | 3.25E−02 | 4.00 |
| 443935 | NM_000236 | Hs. 9994 | lipase, hepatic | 9.52E−02 | 3.98 |
| 458875 | AA814517 | Hs. 321775 | hypothetical protein DKFZp434D1428 | 1.13E−02 | 3.96 |
| 403686 | | | C4001366*: gi\|9837427\|gb\|AAG00570.1\|AF287 | 5.29E−02 | 3.95 |
| 403767 | | | Target Exon | 7.15E−02 | 3.95 |
| 401114 | | | Target Exon | 4.55E−02 | 3.89 |
| 412286 | AW935866 | | gb: QV3-DT0019-120100-055-b04 DT0019 Homo | 2.33E−02 | 3.88 |
| 417505 | T73280 | Hs. 193228 | alanine-glyoxylate aminotransferase 2 | 6.19E−02 | 3.85 |
| 440798 | AB020648 | Hs. 7426 | KIAA0841 protein | 3.29E−02 | 3.85 |
| 403743 | | | C1002604: gi\|8393668\|ref\|NP_058989.1\|kin | 8.30E−02 | 3.81 |
| 429031 | BE002237 | Hs. 239666 | Homo sapiens cDNA FLJ13495 fis, clone PL | 6.17E−02 | 3.79 |
| 453815 | AL135365 | Hs. 126857 | Homo sapiens cDNA FLJ12936 fis, clone NT | 3.29E−02 | 3.77 |
| 423983 | AA333261 | | gb: EST37476 Embryo, 8 week 1 Homo sapien | 4.55E−02 | 3.75 |
| 405206 | | | Target Exon | 7.12E−02 | 3.72 |
| 425057 | AA826434 | Hs. 1619 | achaete-scute complex (Drosophila) homol | 4.55E−02 | 3.72 |
| 446847 | T51454 | Hs. 82845 | Homo sapiens cDNA: FLJ21930 fis, clone H | 4.55E−02 | 3.71 |
| 414535 | W27834 | Hs. 333417 | capping protein (actin filament) muscle | 8.13E−02 | 3.70 |
| 456250 | U09196 | Hs. 82520 | polymerase (DNA-directed), delta 4 | 9.52E−02 | 3.69 |
| 446885 | T74107 | Hs. 122613 | ESTs | 2.31E−02 | 3.67 |
| 409313 | NM_001325 | Hs. 693 | cleavage stimulation factor, 3′pre-RNA, | 2.33E−02 | 3.65 |
| 441159 | AI198604 | Hs. 126682 | ESTs | 4.53E−02 | 3.63 |
| 422519 | AA347877 | Hs. 122730 | ESTs | 5.29E−02 | 3.63 |
| 435545 | AA687415 | Hs. 28107 | ESTs | 1.12E−02 | 3.63 |
| 439910 | H66765 | Hs. 339397 | ESTs | 2.34E−02 | 3.55 |
| 458991 | AI743502 | | gb: wf63h12.x2 Soares_NFL_T_GBC_{—} | 8.30E−02 | 3.54 |
| | | | S1 Homo s | | |
| 403789 | | | NM_001334*: Homo sapiens cathepsin O (CTS | 8.30E−02 | 3.52 |
| 433156 | R59206 | Hs. 17519 | Homo sapiens cDNA: FLJ22539 fis, clone H | 3.29E−02 | 3.52 |
| 404902 | | | NM_025213: Homo sapiens spectrin, beta, n | 5.29E−02 | 3.50 |
| 436499 | AJ276678 | Hs. 283102 | HEF like Protein | 5.29E−02 | 3.50 |
| 424092 | AI085286 | Hs. 133476 | ESTs | 6.19E−02 | 3.38 |
| 458443 | AV647010 | Hs. 27 | glycine dehydrogenase (decarboxylating; | 1.63E−02 | 3.35 |
| 446681 | AJ003624 | Hs. 15896 | kendrin | 4.55E−02 | 3.32 |
| 401778 | | | MCT-1 protein | 8.30E−02 | 3.25 |
| 402244 | | | C19000767*: gi\|4508027\|ref\|NP_003414.1\|z | 6.19E−02 | 3.23 |
| 419287 | X91906 | Hs. 89872 | chloride channel 5 (nephrolithiasis 2, X | 1.64E−02 | 3.22 |
| 405543 | | | C2002468*: gi\|3334856\|emb\|CAA11279.1\|(AJ | 8.17E−02 | 3.20 |
| 429624 | AA458648 | Hs. 99476 | ESTs, Weakly similar to 1313184B alpha 1 | 8.27E−02 | 3.18 |
| 453751 | R36762 | Hs. 101282 | Homo sapiens cDNA: FLJ21238 fis, clone C | 4.55E−02 | 3.17 |
| 404511 | | | NM_004349: Homo sapiens core-binding fact | 1.35E−02 | 3.13 |
| 445159 | AI631036 | Hs. 196843 | ESTs | 3.27E−02 | 3.10 |
| 457565 | BE294029 | Hs. 279903 | Ras homolog enriched in brain 2 | 3.29E−02 | 3.10 |
| 404717 | | | NM_007313*: Homo sapiens v-abl Abelson mu | 6.19E−02 | 3.07 |
| 403920 | | | suppressor of potassium transport defect | 3.29E−02 | 3.07 |
| 445664 | AW968638 | Hs. 237691 | ESTs, Weakly similar to KIAA0601 protein | 5.27E−02 | 3.05 |
| 403442 | | | Target Exon | 6.19E−02 | 3.02 |
| 436336 | AJ011378 | | ESTs | 4.41E−02 | 3.00 |
| 446783 | AW138343 | Hs. 141867 | ESTs | 1.64E−02 | 2.97 |
| 451790 | AA927403 | Hs. 3803 | reticulon 2 | 8.30E−02 | 2.96 |
| 427958 | AA418000 | Hs. 98280 | potassium intermediate/small conductance | 1.62E−02 | 2.92 |
| 430989 | AB009249 | Hs. 248192 | fibroblast growth factor 17 | 8.27E−02 | 2.91 |
| 425744 | AA362985 | | gb: EST72769 Ovary II Homo sapiens cDNA 5 | 8.30E−02 | 2.91 |
| 419511 | AA429750 | Hs. 75113 | general transcription factor IllA | 4.55E−02 | 2.89 |
| 446001 | AI827473 | | ESTs | 6.19E−02 | 2.88 |
| 442602 | R46488 | Hs. 187497 | ESTs | 4.50E−02 | 2.85 |
| 443589 | H52931 | Hs. 165067 | ESTs | 8.27E−02 | 2.85 |
| 457142 | AI924353 | Hs. 290969 | EST | 7.59E−03 | 2.83 |
| 404956 | | | C1003210*: gi\|6912582\|ref\|NP_036524.1\|pe | 3.27E−02 | 2.83 |
| 426181 | AA371422 | Hs. 334371 | hypothetical protein MGC13096 | 6.19E−02 | 2.83 |
| 454086 | AW885909 | Hs. 279789 | PRO1073 protein | 6.19E−02 | 2.82 |
| 450770 | AA019924 | Hs. 28803 | ESTs | 8.30E−02 | 2.81 |
| 452357 | AI638176 | Hs. 283865 | ESTs | 6.19E−02 | 2.80 |
| 456758 | AA325170 | Hs. 224627 | ESTs, Weakly similar to FAHUAA alpha-act | 6.17E−02 | 2.79 |
| 409959 | BE349470 | | mucin 6, gastric | 1.11E−02 | 2.77 |
| 417674 | R07915 | Hs. 20039 | ESTs | 6.19E−02 | 2.75 |
| 446548 | AI769392 | Hs. 200215 | ESTs | 8.30E−02 | 2.75 |
| 407988 | N47760 | Hs. 285107 | hypothetical protein FLJ13397 | 9.24E−03 | 2.70 |
| 435236 | T03890 | Hs. 157208 | ESTs, Highly similar to ARX MOUSE HOMEOB | 9.52E−02 | 2.69 |
| 447090 | AI361114 | Hs. 270711 | ESTs | 7.59E−03 | 2.67 |
| 451068 | AW294432 | Hs. 144252 | ESTs | 3.29E−02 | 2.63 |
| 431668 | AW969610 | Hs. 151179 | ESTs | 3.29E−02 | 2.63 |
| 449000 | U69560 | Hs. 3826 | kelch-like protein C3IP1 | 9.17E−03 | 2.63 |
| 443832 | AI829610 | Hs. 23531 | ESTs | 8.27E−02 | 2.62 |
| 405481 | | | Target Exon | 7.15E−02 | 2.60 |
| 438922 | R71288 | Hs. 259664 | ESTs | 4.53E−02 | 2.60 |
| 402168 | | | NM_022046*: Homo sapiens kallikrein 14 (K | 1.95E−02 | 2.59 |
| 444280 | AI380835 | Hs. 148505 | ESTs | 3.27E−02 | 2.58 |
| 435471 | AA682544 | | ESTs | 4.55E−02 | 2.58 |
| 452137 | AI861840 | Hs. 211687 | ESTs | 2.76E−02 | 2.58 |
| 424049 | AB014524 | Hs. 138380 | KIAA0624 protein | 1.62E−02 | 2.55 |
| 418154 | BE165866 | | nuclear receptor subfamily 1, group I, m | 6.19E−02 | 2.52 |
| 447113 | AA741545 | Hs. 282832 | ESTs, Weakly similar to T24961 hypotheti | 1.13E−02 | 2.50 |
| 439389 | AA318940 | Hs. 56004 | ESTs | 3.77E−02 | 2.48 |
| 442049 | AA310393 | Hs. 190044 | ESTs | 7.15E−02 | 2.47 |
| 445119 | AF035121 | Hs. 12337 | kinase insert domain receptor (a type II | 1.12E−02 | 2.47 |
| 454341 | AI654712 | Hs. 54424 | hepatocyte nuclear factor 4, alpha | 4.48E−02 | 2.45 |
| 420028 | AB014680 | Hs. 8786 | carbohydrate (N-acetylglucosamine-6-O) s | 4.55E−02 | 2.45 |
| 420556 | AA278300 | Hs. 124292 | Homo sapiens cDNA: FLJ23123 fis, clone L | 6.19E−02 | 2.45 |
| 456558 | BE410992 | Hs. 258730 | heme-regulated initiation factor 2-alpha | 6.17E−02 | 2.45 |
| 423954 | AW753164 | Hs. 288604 | KIAA1632 protein | 8.30E−02 | 2.43 |
| 450565 | T72234 | Hs. 193228 | alanine-glyoxylate aminotransferase 2 | 5.29E−02 | 2.40 |
| 446068 | AL049801 | Hs. 13649 | Novel human gene mapping to chomosome 13 | 5.27E−02 | 2.39 |
| 419662 | W87674 | | gb: zh66a01.r1 Soares_fetal_liver_spleen_{—} | 1.55E−02 | 2.38 |
| 432547 | BE350533 | Hs. 306418 | Homo sapiens cDNA FLJ10958 fis, clone PL | 6.19E−02 | 2.38 |
| 403440 | | | Target Exon | 4.55E−02 | 2.37 |
| 407625 | AA148766 | Hs. 293885 | hypothetical protein FLJ14902 | 2.33E−02 | 2.35 |
| 415770 | M79237 | | gb: EST01385 Subtracted Hippocampus, Stra | 7.59E−03 | 2.34 |
| 433224 | AB040919 | Hs. 210958 | KIAA1486 protein | 4.53E−02 | 2.32 |
| 421573 | AI302850 | Hs. 262455 | ESTs | 5.29E−02 | 2.30 |
| 459283 | NM_016940 | Hs. 34136 | chromosome 21 open reading frame 6 | 4.55E−02 | 2.26 |
| 424602 | AK002055 | Hs. 151046 | hypothetical protein FLJ11193 | 8.30E−02 | 2.25 |
| 406180 | | | small inducible cytokine subfamily A (Cy | 3.29E−02 | 2.24 |
| 404810 | | | Target Exon | 8.30E−02 | 2.24 |
| 457841 | AI678031 | Hs. 122813 | ESTs, Weakly similar to ZN22_HUMAN ZINC | 8.30E−02 | 2.24 |
| 458492 | AI143655 | | ESTs | 2.76E−02 | 2.22 |
| 443708 | AI082689 | Hs. 63970 | ESTs | 2.31E−02 | 2.21 |
| 444758 | AL044878 | Hs. 11899 | 3-hydroxy-3-methylglutaryl-Coenzyme A re | 8.30E−02 | 2.21 |
| 454302 | AA306105 | Hs. 50785 | SEC22, vesicle trafficking protein (S. c | 5.29E−02 | 2.20 |
| 456400 | W28090 | | gb: 41h3 Human retina cDNA randomly prime | 6.19E−02 | 2.20 |
| 408568 | AW448965 | Hs. 270087 | ESTs, Weakly similar to I39295 X-linked | 8.30E−02 | 2.18 |
| 444073 | H68409 | Hs. 38715 | ESTs | 8.27E−02 | 2.15 |
| 404437 | | | C1000815: gi\|9653274\|gb\|AAF27628.2\|AF2175 | 8.30E−02 | 2.14 |
| 453315 | BE544203 | Hs. 24831 | ESTs | 6.19E−02 | 2.13 |
| 408931 | AA251995 | Hs. 334648 | poly(A) polymerase alpha | 8.30E−02 | 2.10 |
| 433444 | AW975324 | Hs. 129816 | ESTs | 4.55E−02 | 2.09 |
| 446226 | AW889132 | Hs. 11916 | ribokinase | 4.55E−02 | 2.08 |
| 452266 | AI767250 | Hs. 201446 | ESTs | 8.30E−02 | 2.05 |
| 436869 | NM_014867 | Hs. 5333 | KIAA0711 gene product | 3.86E−02 | 2.04 |
| 433906 | AI167816 | Hs. 43355 | ESTs | 5.29E−02 | 2.02 |
| 402370 | | | Target Exon | 8.30E−02 | 2.02 |
| 434306 | AW081757 | Hs. 44241 | Homo sapiens cDNA: FLJ21447 fis, clone C | 6.19E−02 | 2.01 |
| 422721 | R41154 | Hs. 275711 | hypothetical protein MGC2452 | 6.19E−02 | 2.01 |

Pkey: Unique Eos probeset identifier number
ExAccn: Exemplar Accession number, Genbank accession number
UnigeneID: Unigene number
Unigene Title: Unigene gene title
R1: Wilcoxon rank-sum test p-value
R2: Trimean of responders over Trimean of non-responders Ratio

TABLE 10B

| Pkey | CAT Number | Accession |
|---|---|---|
| 421911 | 863300_1 | AL041520 AA300086 |
| 455191 | 1107840_1 | AW864356 AW864547 AW864277 AW864496 |
| 442552 | 3248_3 | BE566571 BE143063 BF367361 AW954369 AA412572 W95547 BE005367 |
| | | AA374200 AI769772 AA809852 AI918702 AW025682 AI272033 |
| | | AA713679 AI638383 Al364631 AW518885 N51823 AA256588 N47774 BE707161 |
| | | BF980035 AI628605 AW292949 BE350976 AW263078 AI885558 |
| | | AI261878 BE671974 N51071 AI695006 BM264289 BG055084 AI922512 AA633530 BM263987 |
| | | N56889 AW075473 BE700841 AW023886 BF527967 |
| | | AW591478 BE669905 N51720 AA806021 AA846566 W95290 AI453800 AI783684 AI417702 AW086132 |
| | | BE552047 AA227699 AW076131 AI126790 |
| | | AI654491 H01465 BF437982 AA400937 R20624 BF825527 H02691 W56480 BF944773 |
| | | R21195 R82844 H02596 BE243273 H50040 |
| 411047 | 1067666_1 | AW850678 AW938479 AW814826 |
| 412286 | 210181_1 | AW935866 BE069200 BF838900 BG989737 BG989811 BF833185 BF837808 |
| 423983 | 888204_1 | AA333365 AA552870 AA333261 |
| 458991 | 3076691_1 | AI743502 AI807438 |
| 436336 | 53728_1 | AJ011378 AA416944 AA469902 AI015437 |
| 425744 | 320952_2 | AW963337 AA362985 T27244 |
| 446001 | 647827_1 | AI827473 AW510751 AW182908 AI269760 |
| 409959 | 2781_3 | AW513804 BE179199 BE179195 BE179198 BE179204 BE162686 |
| 435471 | 131589_1 | AA682544 AI248010 AA703577 |
| 418154 | 23972_1 | X56199 AU119734 AW967136 W38705 W90159 AW444647 BF094079 |
| | | BE694692 AA339496 AA029093 BG925977 AA401072 AA348138 N90874 |
| | | BF055081 BF111983 BF091812 AI399764 BE931041 BF999882 BF352250 |
| | | BI049436 AA010244 AW374411 H52942 BF364437 AL538880 |
| | | AU137031 BE218742 BE175700 BE708041 AV651847 AV649491 AV649227 |
| | | AV649164 AV698455 AV651656 M78602 BF910285 AA333298 H75493 |
| | | AA332039 BM463335 AL603109 BF753137 BF083242 AI309717 AI349651 |
| | | BE174377 AW361207 BE159533 AL603126 BI003025 AA319706 |
| | | AW903950 AU118470 AU135122 AI811901 AA401166 BE165866 BE165832 |
| | | AA319621 BF749419 AI903726 BE833899 BI061305 BI061311 H78857 |
| | | BF912227 BF912224 AV747164 AA126644 W17094 T05342 AW374665 AA091968 |
| | | AA095633 BI009746 AL038020 BE010083 BE008038 BF808424 |
| | | BE010133 BF364245 BF328029 BE010189 AW903901 AW844209 AW879041 AL037999 |
| | | AV690829 AV693074 AV693056 AV690862 BE159515 |
| | | BE074613 AW905394 |
| 419662 | 2431548_1 | W87872 W87674 W87774 |
| 415770 | 2051914_1 | AA169260 AI267298 M79237 |
| 458492 | 2797412_1 | AI869287 AI143655 |
| 456400 | 2391890_1 | W27943 W28090 |

Pkey: Unique Eos probeset identifier number
CAT number: Gene cluster number
Accession: Genbank accession numbers

TABLE 10C

| Pkey | Ref | Strand | Nt_position |
|---|---|---|---|
| 404354 | 7630858 | Minus | 115516-115702 |
| 404483 | 8096904 | Minus | 162212-163710 |
| 401197 | 9719705 | Plus | 176341-176452 |
| 401635 | 7145001 | Minus | 51895-52764 |
| 406570 | 3983519 | Minus | 88469-88570 |
| 403609 | 8308266 | Minus | 125974-126320 |
| 403686 | 7387348 | Minus | 66625-68364 |
| 403767 | 7767782 | Plus | 142903-143081 |
| 401114 | 9966554 | Plus | 52327-53385 |
| 403743 | 7652003 | Minus | 136463-136646 |
| 405206 | 6692345 | Plus | 17807-18338, 20430-20538 |
| 403789 | 8084957 | Minus | 64056-64148, 65262-65425 |
| 404902 | 7331453 | Plus | 36243-36358 |
| 401778 | 7249133 | Minus | 136881-137147, 137499-137628, |
| | | | 137858-13814 |
| 402244 | 7690152 | Plus | 36938-38272 |
| 405543 | 9857582 | Minus | 104338-104449 |
| 404511 | 8151864 | Minus | 148501-148659 |
| 404717 | 9838068 | Minus | 165900-166052, 169415-169599, |
| | | | 171430-17160 |
| 403920 | 7710868 | Plus | 40312-40490 |
| 403442 | 7210003 | Plus | 174560-175270 |
| 404956 | 7387343 | Plus | 55883-56203 |
| 405481 | 3688109 | Plus | 5718-5837, 8719-8818 |
| 402168 | 7458725 | Minus | 43245-43397 |
| 403440 | 9743372 | Minus | 34592-34661, 41940-42100 |
| 406180 | 7283201 | Minus | 38923-39107 |
| 404810 | 3702433 | Minus | 65278-65642 |
| 404437 | 5354481 | Minus | 17773-17938, 20263-20429 |
| 402370 | 9558580 | Minus | 14525-15733 |

Pkey: Unique number corresponding to an Eos probeset
Ref: Sequence source. The 7 digit numbers in this column are Genbank Identifier (GI) numbers. “Dunham, et al.” refers to the publication entitled “The DNA sequence of human chromosome 22” Dunham. et al. (1999) Nature 402: 489-495.
Strand: Indicates DNA strand from which exons were predicted.
Nt_position: Indicates nucleotide positions of predicted exons.

Table 11A lists about 2410 genes up-regulated in Hepatitis C positive liver tissues compared to Hepatitis C negative liver tissues. These genes have the potential to be diagnostics and/or prognostic markers for Hepatitis C infected liver tissues. They may also provide clinical information on Hepatitis C infection and pathology. They may also be potential targets for therapeutic drugs and/or treatments. These were selected from 59680 probesets on the Affymetrix/Eos Hu03 GeneChip array such that the t-test p-value between the Hepatitis C positive liver tissues group and the Hepatitis C negative liver tissues was less than 0.01, and that the differences between the average “standardized values” of the different Hepatitis C positive liver tissues was equal to or above 1.0 more than the average standardized values of the different Hepatitis C negative liver tissues. The “standardized values” of the Hepatitis C liver tissues were derived by subtracting the median of the several different Hepatitis C negative liver tissue values from each probeset value, then divided by the interquantile range (IQR) of the same Hepatitis C negative liver tissue values.

TABLE 11 A

| Pkey | ExAccn | UnigeneID | Unigene Title | R1 | R2 |
|---|---|---|---|---|---|
| 413278 | BE563085 | Hs. 833 | interferon-stimulated protein, 15 kDa | 1.24E−10 | 13.42 |
| 447164 | AF026941 | Hs. 17518 | vipirin; similar to inflammatory respon | 1.68E−07 | 11.03 |
| 426312 | AF026939 | Hs. 181874 | interferon-induced protein with tetratri | 8.68E−10 | 9.84 |
| 431629 | AU077025 | Hs. 265827 | interferon, alpha-inducible protein (clo | 9.17E−13 | 9.06 |
| 422596 | AF063611 | Hs. 118633 | 2′-5′-oligoadenylate synthetase−like | 4.59E−09 | 8.28 |
| 426711 | AA383471 | Hs. 343800 | conserved gene amplified in osteosarcoma | 3.15E−15 | 6.94 |
| 427579 | AA366143 | Hs. 179669 | hypothetical protein FLJ20637 | 4.05E−09 | 6.76 |
| 409461 | AA382169 | Hs. 54483 | N-myc (and STAT) interactor | 5.26E−12 | 6.61 |
| 453779 | N35187 | Hs. 43388 | 28 kD interferon responsive protein | 1.47E−11 | 6.48 |
| 418396 | AI765805 | Hs. 26691 | ESTs | 4.41E−08 | 6.33 |
| 433364 | AI075407 | Hs. 296083 | ESTs, Moderately similar to I54374 gene | 2.97E−15 | 6.27 |
| 414404 | W16712 | | KIAA0306 protein | 1.42E−06 | 6.25 |
| 455705 | AW161061 | | ESTs, Weakly similar to zinc finger prot | 5.43E−11 | 6.24 |
| 430478 | NM_014349 | Hs. 241535 | apolipoprotein L, 3 | 3.88E−17 | 6.09 |
| 413880 | AI660842 | Hs. 110915 | interleukin 22 receptor | 5.13E−05 | 5.91 |
| 417621 | AV654694 | Hs. 82316 | interferon-induced, hepatitis C-associat | 2.55E−12 | 5.55 |
| 432680 | T47364 | Hs. 278613 | interferon, alpha-inducible protein 27 | 2.59E−15 | 5.35 |
| 418622 | AW971960 | Hs. 236456 | ESTs, Weakly similar to A46010 X-linked | 9.09E−04 | 5.26 |
| 448569 | BE382657 | Hs. 21486 | signal transducer and activator of trans | 1.56E−15 | 5.00 |
| 421379 | Y15221 | Hs. 103982 | small inducible cytokine subfamily B (Cy | 9.16E−09 | 4.97 |
| 414004 | AA737033 | Hs. 7155 | ESTs, Moderately similar to 2115357A TYK | 3.33E−10 | 4.73 |
| 414760 | BE298063 | Hs. 77254 | chromobox homolog 1 (Drosophila HP1 beta | 3.99E−10 | 4.66 |
| 446217 | AI651594 | Hs. 99709 | ESTs | 2.36E−09 | 4.63 |
| 431620 | AA126109 | Hs. 264981 | 2′-5′-oligoadenylate synthetase 2 (69-71 | 4.54E−12 | 4.57 |
| 414737 | AI160386 | Hs. 125087 | ESTs | 4.78E−06 | 4.43 |
| 421904 | BE143533 | Hs. 109309 | hypothetical protein FLJ20035 | 2.86E−10 | 4.36 |
| 402994 | | | NM_002463*: Homo sapiens myxovirus (influ | 7.92E−05 | 4.29 |
| 428467 | AK002121 | Hs. 184465 | hypothetical protein FLJ11259 | 2.14E−04 | 4.28 |
| 409703 | NM_006187 | Hs. 56009 | 2′-5′-oligoadenylate synthetase 3 (100 k | 4.28E−14 | 4.25 |
| 438154 | AI671957 | Hs. 19523 | Homo sapiens clone 022f05 My030 protein | 6.70E−09 | 4.13 |
| 437938 | AI950087 | | gb: wq05c02.x1 NCI_CGAP_Kid12 Homo sapien | 1.51E−08 | 4.08 |
| 432204 | AI916132 | Hs. 121593 | Homo sapiens cDNA FLJ13123 fis, clone NT | 6.22E−09 | 4.07 |
| 406659 | AA663985 | Hs. 277477 | major histocompatibility complex, class | 1.50E−06 | 4.06 |
| 406621 | X57809 | Hs. 8997 | immunoglobulin lambda locus | 1.85E−04 | 4.03 |
| 414915 | NM_002462 | Hs. 76391 | myxovirus (influenza) resistance 1, homo | 2.02E−12 | 4.03 |
| 435665 | AI248952 | Hs. 12320 | ESTs | 3.98E−07 | 4.01 |
| 442700 | AA377618 | Hs. 103834 | hypothetical protein MGC5576 | 2.54E−07 | 3.96 |
| 410240 | AL157424 | Hs. 61289 | synaptojanin 2 | 7.09E−06 | 3.95 |
| 447747 | AW853144 | Hs. 49203 | ESTs | 1.01E−03 | 3.88 |
| 452194 | AI694413 | | Ubiquitin-like protein FAT10??? - diubiq | 4.53E−08 | 3.85 |
| 409614 | BE297412 | Hs. 55189 | hypothetical protein | 1.22E−08 | 3.85 |
| 419071 | AL137330 | Hs. 301055 | hypothetical protein DKFZp434F0272 | 7.16E−06 | 3.80 |
| 427283 | AL119796 | Hs. 174185 | ectonucleotide pyrophosphatase/phosphodi | 6.28E−07 | 3.78 |
| 437834 | AA769294 | | gb: nz36g03.s1 NCI_CGAP_GCB1 Homo sapiens | 9.13E−06 | 3.75 |
| 419200 | AW966405 | | prefoldin 5 | 1.36E−09 | 3.75 |
| 419150 | T29618 | Hs. 89640 | TEK tyrosine kinase, endothelial (venous | 8.23E−04 | 3.74 |
| 434210 | AA665612 | | ESTs | 2.80E−10 | 3.73 |
| 427094 | AB025254 | Hs. 283761 | tudor repeat associator with PCTAIRE 2 | 3.92E−10 | 3.71 |
| 407151 | H25836 | Hs. 301527 | ESTs, Moderately similar to unknown [H. s | 1.13E−09 | 3.69 |
| 427562 | R56424 | Hs. 134191 | ESTs | 1.84E−06 | 3.67 |
| 452690 | AI536070 | Hs. 15085 | ESTs | 5.53E−05 | 3.64 |
| 416126 | R66415 | | gb: yh01a04.s2 Soares infant brain 1NIB H | 7.65E−03 | 3.64 |
| 442439 | U09759 | Hs. 246857 | mitogen-activated protein kinase 9 | 1.51E−07 | 3.62 |
| 443378 | AW392550 | Hs. 9280 | proteasome (prosome, macropain) subunit, | 4.69E−10 | 3.62 |
| 419550 | D50918 | Hs. 90998 | KIAA0128 protein; septin 2 | 2.13E−10 | 3.57 |
| 451578 | NM_016323 | Hs. 26663 | cyclin-E binding protein 1 | 1.88E−08 | 3.54 |
| 419438 | AA406400 | Hs. 12482 | glyceronephosphate O-acyltransferase | 2.63E−04 | 3.53 |
| 409899 | U07418 | Hs. 57301 | mutL (E. coli) homolog 1 (colon cancer, | 7.69E−05 | 3.51 |
| 441423 | AI793299 | Hs. 126877 | ESTs | 4.67E−06 | 3.50 |
| 409153 | W03754 | Hs. 50813 | hypothetical protein FLJ20022 | 9.52E−06 | 3.50 |
| 448128 | U71267 | Hs. 20423 | CCR4-NOT transcription complex, subunit | 5.13E−09 | 3.48 |
| 409893 | AW247090 | Hs. 57101 | minichromosome maintenance deficient (S. | 1.21E−06 | 3.48 |
| 444342 | NM_014398 | Hs. 10887 | similar to lysosome-associated membrane | 7.46E−09 | 3.43 |
| 406906 | Z25424 | | gb: H. sapiens protein-serine/threonine ki | 5.22E−05 | 3.42 |
| 409512 | AW979187 | Hs. 293591 | melanoma differentiation associated prot | 7.83E−13 | 3.41 |
| 416844 | AA299330 | Hs. 1042 | Sjogren syndrome antigen A1 (52 kD, ribon | 3.64E−08 | 3.40 |
| 446452 | AB018283 | Hs. 15099 | KIAA0740 gene product | 3.88E−07 | 3.39 |
| 448111 | AA053486 | Hs. 20315 | interferon-induced protein with tetratri | 3.07E−11 | 3.39 |
| 400244 | | | Eos Control | 1.17E−09 | 3.36 |
| 433409 | AI278802 | Hs. 25661 | ESTs | 2.37E−03 | 3.33 |
| 408411 | C15118 | Hs. 322482 | hypothetical protein DKFZp566J2046 | 2.97E−08 | 3.31 |
| 408393 | AW015318 | Hs. 23165 | ESTs | 6.98E−08 | 3.29 |
| 439971 | W32474 | Hs. 301746 | RAP2A, member of RAS oncogene family | 1.02E−08 | 3.28 |
| 453023 | AW028733 | Hs. 31439 | serine protease inhibitor, Kunitz type, | 7.29E−06 | 3.27 |
| 428706 | AA432030 | Hs. 265827 | interferon, alpha-inducible protein (clo | 1.46E−08 | 3.26 |
| 424865 | AF011333 | Hs. 153563 | lymphocyte antigen 75 | 2.38E−06 | 3.22 |
| 422598 | W93032 | Hs. 325568 | ESTs, Weakly similar to A46010 X-linked | 9.27E−13 | 322 |
| 423436 | R21176 | Hs. 100926 | ESTs | 2.71E−03 | 3.21 |
| 418918 | X07871 | Hs. 89476 | CD2 antigen (p50), sheep red blood cell | 3.03E−09 | 3.20 |
| 438769 | AA830684 | Hs. 163426 | ESTs | 4.96E−05 | 3.19 |
| 458379 | T78886 | Hs. 284450 | ESTs | 2.70E−05 | 3.17 |
| 426572 | AB037783 | Hs. 170623 | hypothetical protein FLJ11183 | 1.66E−03 | 3.15 |
| 419257 | X53461 | Hs. 89781 | upstream binding transcription factor, R | 7.32E−07 | 3.13 |
| 422746 | NM_004484 | Hs. 119651 | glypican 3 | 7.65E−06 | 3.13 |
| 427657 | AV652249 | Hs. 180107 | polymerase (DNA directed), beta | 7.44E−12 | 3.10 |
| 433679 | AA393968 | Hs. 180145 | HSPC030 protein | 2.98E−04 | 3.09 |
| 418838 | AW385224 | Hs. 35198 | ectonucleotide pyrophosphatase/phosphodi | 3.18E−06 | 3.07 |
| 417678 | X06560 | Hs. 82396 | 2′,5′-oligoadenylate synthetase 1 (40-46 | 1.67E−09 | 3.06 |
| 428227 | AA321649 | Hs. 2248 | small inducible cytokine subfamily B (Cy | 3.03E−12 | 3.06 |
| 408989 | AW361666 | Hs. 49500 | KIAA0746 protein | 1.59E−06 | 3.03 |
| 410340 | AW182833 | Hs. 112188 | hypothetical protein FLJ13149 | 3.80E−05 | 3.03 |
| 417389 | BE260964 | Hs. 82045 | midkine (neurite growth-promoting factor | 3.24E−07 | 3.01 |
| 423461 | AB020527 | Hs. 128827 | solute carrier family 17 (sodium phospha | 3.52E−06 | 3.00 |
| 411837 | AW866282 | | gb: QV4-SN0024-080400-167-a03 SN0024 Homo | 1.26E−03 | 3.00 |
| 427528 | AU077143 | Hs. 179565 | minichromosome maintenance deficient (S. | 1.35E−06 | 2.99 |
| 420058 | AK001423 | Hs. 94694 | Homo sapiens cDNA FLJ10561 fis. clone NT | 1.16E−06 | 2.99 |
| 432967 | AA572949 | Hs. 207566 | ESTs | 2.85E−03 | 2.98 |
| 415653 | AA165400 | Hs. 5321 | hypothetical protein R33729_1 | 1.33E−04 | 2.95 |
| 419329 | AY007220 | Hs. 288998 | S100-type calcium binding protein A14 | 1.31E−04 | 2.94 |
| 450937 | R49131 | Hs. 26267 | ATP-dependant interferon response protei | 1.74E−08 | 2.93 |
| 414617 | AI339520 | Hs. 288817 | ESTs, Moderately similar to N Chain N, M | 5.82E−05 | 2.92 |
| 450236 | AW162998 | Hs. 24684 | KIAA1376 protein | 1.00E−03 | 2.91 |
| 413179 | N99692 | Hs. 75227 | Target CAT | 2.78E−03 | 2.90 |
| 436746 | AA730045 | Hs. 187866 | ESTs | 1.29E−05 | 2.89 |
| 448879 | Z44154 | Hs. 22394 | hypothetical protein FLJ10893 | 3.34E−06 | 2.88 |
| 414052 | AW578849 | Hs. 283552 | ESTs, Weakly similar to unnamed protein | 7.88E−08 | 2.87 |
| 408080 | AW149754 | Hs. 248652 | ESTs, Weakly similar to T00273 hypotheti | 5.99E−07 | 2.87 |
| 406990 | S82075 | | gb: PA4 = candidate oncogene {3′region} [h | 1.14E−05 | 2.86 |
| 409444 | H47933 | Hs. 33983 | ESTs, Weakly similar to ALU6_HUMAN ALU S | 6.32E−05 | 2.85 |
| 453883 | AI638516 | Hs. 347524 | cofactor required for Sp1 transcriptiona | 3.20E−09 | 2.85 |
| 434130 | AL036067 | Hs. 18925 | protein x 0001 | 4.32E−05 | 2.84 |
| 408510 | AA731642 | Hs. 285017 | hypothetical protein FLJ21799 | 8.74E−06 | 2.84 |
| 409231 | AA446644 | Hs. 692 | GA733-2 antigen; epithelial glycoprotein | 1.04E−04 | 2.83 |
| 448465 | AW500269 | Hs. 21264 | KIAA0782 protein | 8.90E−03 | 2.83 |
| 422440 | NM_004812 | Hs. 116724 | aldo-keto reductase family 1, member B10 | 1.65E−03 | 2.83 |
| 432378 | AI493046 | Hs. 146133 | ESTs | 5.01E−07 | 2.82 |
| 432279 | N95104 | Hs. 274260 | ATP-binding cassette, sub-family C (CFTR | 8.30E−08 | 2.81 |
| 443119 | AA312264 | Hs. 7980 | hypothetical protein MGC12966 | 6.95E−06 | 2.80 |
| 402264 | | | NM_020657*: Homo sapiens zinc finger prot | 4.15E−04 | 2.75 |
| 422112 | BE540240 | Hs. 111783 | Lsm1 protein | 3.36E−06 | 2.74 |
| 422553 | AI697720 | Hs. 171455 | ESTs, Weakly similar to T31613 hypotheti | 2.97E−05 | 2.74 |
| 432401 | NM_013330 | Hs. 274479 | NME7 | 5.36E−05 | 2.73 |
| 442607 | AA507576 | Hs. 288361 | Homo sapiens cDNA: FLJ22696 fis, clone H | 4.35E−05 | 2.73 |
| 448550 | AL161983 | Hs. 21415 | Homo sapiens mRNA; cDNA DKFZp761K2024 (f | 2.37E−06 | 2.73 |
| 439237 | AW408158 | Hs. 318893 | ESTs, Weakly similar to A47582 B-cell gr | 5.16E−08 | 2.73 |
| 417308 | H60720 | Hs. 81892 | KIAA0101 gene product | 3.87E−07 | 2.71 |
| 433489 | AW629150 | | ESTs | 2.41E−04 | 2.68 |
| 433437 | U20536 | Hs. 3280 | caspase 6, apoptosis-related cysteine pr | 1.32E−07 | 2.67 |
| 452327 | AK000196 | Hs. 29052 | hypothetical protein FLJ20189 | 1.23E−06 | 2.67 |
| 447880 | AA214240 | Hs. 301226 | KIAA1085 protein | 6.35E−08 | 2.65 |
| 430308 | BE540865 | Hs. 238990 | cyclin-dependent kinase inhibitor 1B (p2 | 5.39E−08 | 2.65 |
| 431967 | AJ243653 | Hs. 283404 | organic cation transporter | 6.95E−03 | 2.62 |
| 425342 | AA534073 | Hs. 155751 | voltage-dependent anion channel 3 | 2.81E−05 | 2.61 |
| 441737 | X79449 | Hs. 7957 | adenosine deaminase, RNA-specific | 1.72E−10 | 2.61 |
| 431939 | AW008061 | Hs. 231994 | ESTs | 1.34E−04 | 2.60 |
| 456463 | AA380180 | Hs. 56896 | ESTs | 3.87E−03 | 2.60 |
| 407756 | AA116021 | Hs. 38260 | ubiquitin specific protease 18 | 1.46E−11 | 2.59 |
| 411060 | NM_006074 | Hs. 318501 | Homo sapiens mRNA full length insert cDN | 1.27E−12 | 2.58 |
| 435839 | AF249744 | Hs. 25951 | Rho guanine nucleotide exchange factor | 7.50E−03 | 2.57 |
| 413431 | AW246428 | Hs. 75355 | (ubiquitin-conjugating enzyme E2N (homolo | 4.19E−03 | 2.57 |
| 411605 | AW006831 | | ESTs | 1.58E−04 | 257 |
| 423545 | AP000692 | Hs. 129781 | chromosome 21 open reading frame 5 | 7.99E−04 | 2.57 |
| 453917 | BE297466 | Hs. 36587 | protein phosphatase 1, regulatory subuni | 1.90E−06 | 2.57 |
| 429680 | AL035754 | Hs. 2474 | toll-like receptor 1 | 9.61E−06 | 2.56 |
| 449718 | AA459480 | Hs. 23956 | hypothetical protein FLJ20502 | 6.31E−07 | 2.56 |
| 433163 | R40468 | Hs. 163582 | ESTs | 1.53E−03 | 2.56 |
| 423753 | Y11312 | Hs. 132463 | phosphoinositide−3-kinase, class 2, beta | 1.90E−05 | 2.55 |
| 419082 | AW953977 | Hs. 78825 | matrin3 | 3.07E−05 | 2.54 |
| 450698 | W31489 | Hs. 95044 | ESTs, Weakly similar to I38022 hypotheti | 8.19E−05 | 2.54 |
| 447400 | AK000322 | Hs. 18457 | hypothetical protein FLJ20315 | 1.76E−10 | 2.54 |
| 426172 | AA371307 | Hs. 125056 | ESTs | 9.68E−06 | 2.54 |
| 431685 | AW296135 | Hs. 267659 | vav 3 oncogene | 1.05E−06 | 2.54 |
| 423617 | AA741058 | Hs. 20065 | ESTs | 3.05E−06 | 2.54 |
| 413670 | AB000115 | Hs. 75470 | hypothetical protein, expressed in osteo | 6.06E−08 | 2.53 |
| 449613 | N63808 | Hs. 34299 | ESTs | 4.88E−06 | 2.53 |
| 432388 | X15218 | Hs. 2969 | v-ski avian sarcoma viral oncogene homol | 2.62E−04 | 2.51 |
| 416795 | AI497778 | Hs. 20509 | HBV pX associated protein-8 | 7.52E−05 | 2.51 |
| 409259 | AW608930 | Hs. 52184 | hypothetical protein FLJ20618 | 1.37E−09 | 2.51 |
| 435017 | AA336522 | Hs. 12854 | angiotensin II, type I receptor-associat | 1.94E−08 | 2.51 |
| 420292 | AW991781 | | gb: QV2-BN0007-280300-104-d06 BN0007 Homo | 3.31E−03 | 2.51 |
| 410582 | AW867197 | Hs. 337561 | hypothetical protein FLJ21616 | 5.40E−07 | 2.50 |
| 449478 | AA829848 | Hs. 193059 | ESTs | 3.55E−03 | 2.50 |
| 409346 | AL162066 | Hs. 54320 | hypothetical protein DKFZp762D096 | 6.84E−08 | 2.50 |
| 418416 | U11700 | Hs. 84999 | ATPase, Cu transporting, beta polypeptid | 3.76E−08 | 2.49 |
| 448570 | AI923944 | Hs. 30913 | ESTs | 8.37E−06 | 2.47 |
| 418476 | AA648431 | Hs. 37883 | hypothetical protein PNAS-131 | 6.13E−05 | 2.46 |
| 432710 | AA609685 | Hs. 278672 | membrane component, chromosome 11, surfa | 8.84E−08 | 2.45 |
| 442679 | R53718 | | hypothetical protein FLJ10659 | 2.05E−07 | 2.45 |
| 416309 | R84694 | Hs. 79194 | cAMP responsive element binding protein | 6.20E−06 | 2.45 |
| 424090 | X99699 | Hs. 139262 | XIAP associated factor-1 | 1.47E−06 | 2.44 |
| 448901 | AK001021 | Hs. 22505 | hypothetical protein FLJ10159 | 1.09E−08 | 2.44 |
| 429635 | AW968360 | Hs. 14355 | Homo sapiens cDNA FLJ13207 fis, clone NT | 2.80E−03 | 2.44 |
| 422173 | BE385828 | Hs. 250619 | phorbolin-like protein MDS019 | 2.07E−08 | 2.44 |
| 439708 | AI761369 | Hs. 59584 | hypothetical protein FLJ21144 | 4.54E−05 | 2.43 |
| 449239 | T24653 | Hs. 23360 | likely ortholog of yeast ARV1 | 1.44E−06 | 2.43 |
| 441327 | AK001706 | Hs. 7778 | hypothetical protein FLJ10751 | 1.41E−07 | 2.43 |
| 407366 | AF026942 | Hs. 17518 | gb: Homo sapiens cig33 mRNA, partial sequ | 7.03E−06 | 2.43 |
| 412898 | AI129903 | Hs. 74669 | vesicle-associated membrane protein 5 (m | 1.35E−04 | 2.42 |
| 452355 | N54926 | Hs. 29202 | G protein-coupled receptor 34 | 1.31E−05 | 2.42 |
| 446526 | H89616 | | Homo sapiens cDNA FLJ13357 fis, clone PL | 9.78E−04 | 2.42 |
| 421915 | AW207000 | Hs. 126857 | Homo sapiens cDNA FLJ12936 fis, clone NT | 7.84E−06 | 2.41 |
| 443486 | NM_003428 | Hs. 9450 | zinc finger protein 84 (HPF2) | 4.22E−03 | 2.40 |
| 457465 | AW301344 | Hs. 122908 | DNA replication factor | 1.19E−04 | 2.40 |
| 439718 | AA307634 | Hs. 6650 | vacuolar protein sorting 45B (yeast homo | 6.66E−08 | 2.40 |
| 440874 | NM_003188 | Hs. 7510 | mitogen-activated protein kinase kinase | 5.30E−05 | 2.40 |
| 420137 | AA306478 | Hs. 95327 | CD3D antigen, delta polypeptide (TiT3 co | 1.30E−05 | 2.40 |
| 429164 | AI688663 | Hs. 116586 | ESTs | 4.14E−04 | 2.40 |
| 427484 | N32859 | Hs. 37288 | nuclear receptor subfamily 1, group D, m | 1.58E−07 | 2.39 |
| 413815 | AL046341 | Hs. 75562 | discoidin domain receptor family, member | 5.22E−04 | 2.39 |
| 452835 | AK001269 | Hs. 30738 | hypothetical protein FLJ10407 | 2.27E−09 | 2.39 |
| 414669 | Z83850 | Hs. 76838 | serine (or cysteine) proteinase inhibito | 2.67E−13 | 2.39 |
| 409154 | U72882 | Hs. 50842 | interferon-induced protein 35 | 2.65E−09 | 2.39 |
| 420952 | AA282067 | Hs. 88972 | ESTs, Moderately similar to A46010 X-lin | 8.44E−04 | 2.39 |
| 416068 | R45985 | Hs. 115175 | sterile-alpha motif and leucine zipper c | 2.72E−03 | 2.38 |
| 450607 | AL050373 | Hs. 25213 | hypothetical protein | 2.15E−06 | 2.38 |
| 421040 | AA715026 | Hs. 135280 | ESTs | 1.18E−06 | 2.38 |
| 450206 | AI796450 | Hs. 201600 | ESTs | 4.49E−06 | 2.38 |
| 426805 | AB032945 | Hs. 172506 | myosin VB | 8.69E−05 | 2.37 |
| 436436 | AI417516 | Hs. 202091 | ESTs | 6.37E−04 | 2.37 |
| 428928 | BE409838 | Hs. 194657 | cadherin 1, type 1, E-cadherin (epitheli | 3.30E−07 | 2.37 |
| 414489 | AI620677 | Hs. 73105 | ESTs | 3.01E−08 | 2.37 |
| 443854 | AI089079 | Hs. 135214 | hypothetical protein FLJ12800 | 3.53E−05 | 2.36 |
| 402424 | | | NM_024901: Homo sapiens hypothetical prot | 1.01E−05 | 2.36 |
| 451752 | AB032997 | | KIAA1171 protein | 1.18E−14 | 2.36 |
| 435458 | F11872 | Hs. 4892 | Homo sapiens clone 24841 mRNA sequence | 2.25E−03 | 2.36 |
| 428180 | AI129767 | Hs. 182874 | guanine nucleotide binding protein (G pr | 2.38E−05 | 2.35 |
| 448760 | AA313825 | Hs. 323583 | AD036 protein | 1.47E−07 | 2.35 |
| 425235 | AA353113 | Hs. 112497 | Homo sapiens cDNA: FLJ22743 fis, clone H | 2.65E−06 | 2.34 |
| 404649 | | | Target Exon | 1.06E−08 | 2.34 |
| 420174 | AI824144 | Hs. 199749 | ESTs | 5.79E−07 | 2.34 |
| 423258 | L13460 | Hs. 1644 | cytochrome P450, subfamily VIIA (cholest | 1.20E−05 | 2.34 |
| 444324 | AI301330 | Hs. 143838 | ESTs | 3.55E−05 | 2.33 |
| 414283 | AW960011 | Hs. 154993 | ESTs | 1.70E−07 | 2.33 |
| 412802 | U41518 | Hs. 74602 | aquaporin 1 (channel-forming integral pr | 5.06E−04 | 2.33 |
| 425375 | AA631977 | Hs. 155995 | KIAA0643 protein | 4.34E−05 | 2.33 |
| 428514 | AW236861 | Hs. 193139 | ESTs | 6.38E−09 | 2.32 |
| 423645 | AI215632 | Hs. 147487 | ESTs | 5.17E−06 | 2.32 |
| 448222 | AI648587 | Hs. 20725 | Mov10 (Moloney teukemia virus 10, mouse) | 7.56E−06 | 2.31 |
| 422633 | X56832 | Hs. 118804 | enolase 3, (beta, muscle) | 2.83E−09 | 2.31 |
| 429756 | AB022663 | Hs. 215857 | ring finger protein 14 | 2.40E−07 | 2.31 |
| 428342 | AI739168 | | Homo sapiens cDNA FLJ13458 fis, clone PL | 5.59E−06 | 2.31 |
| 421246 | AW582962 | Hs. 102897 | CGI-47 protein | 1.36E−06 | 2.30 |
| 442821 | BE391929 | Hs. 8752 | transmembrane protein 4 | 7.86E−06 | 2.30 |
| 410541 | AA065003 | Hs. 64179 | syntenin-2 protein | 2.03E−03 | 2.29 |
| 441872 | BE567100 | Hs. 154938 | hypothetical protein MDS025 | 2.15E−09 | 2.29 |
| 423804 | AW403448 | Hs. 16725 | interferon-stimulated transcription fact | 3.25E−07 | 2.28 |
| 411049 | AW814885 | Hs. 19553 | ESTs | 6.16E−05 | 2.28 |
| 426272 | AW450671 | Hs. 189284 | ESTs | 6.41E−06 | 2.28 |
| 440975 | AW499914 | Hs. 7579 | hypothetical protein FLJ10402 | 6.22E−07 | 2.28 |
| 447651 | T60407 | Hs. 217882 | ESTs, Moderately similar to ALU1_HUMAN A | 5.50E−08 | 2.28 |
| 410577 | X91911 | Hs. 64639 | glioma pathogenesis-related protein | 8.61E−07 | 2.27 |
| 433309 | AA807060 | Hs. 126558 | ESTs | 2.10E−03 | 2.26 |
| 447973 | AB011169 | Hs. 20141 | similar to S. cerevisiae SSM4 | 1.75E−06 | 2.26 |
| 426437 | BE076537 | Hs. 169895 | ubiquitin-conjugating enzyme E2L 6 | 1.56E−09 | 2.26 |
| 452744 | AI267652 | Hs. 246107 | Homo sapiens mRNA; cDNA DKFZp434E082 (fr | 4.59E−11 | 2.25 |
| 418452 | BE379749 | Hs. 85201 | C-type (calcium dependent, carbohydrate- | 4.78E−11 | 2.25 |
| 420245 | AA256902 | Hs. 252433 | Homo sapiens cDNA FLJ13794 fis, clone TH | 4.55E−05 | 2.24 |
| 431677 | AK000496 | Hs. 306989 | hypothetical protein FLJ20489 | 2.67E−05 | 2.24 |
| 421902 | BE392717 | | gb: 601307571F1 NIH_MGC_44 Homo sapiens c | 1.28E−04 | 2.24 |
| 413869 | NM_000878 | Hs. 75596 | interleukin 2 receptor, beta | 2.47E−07 | 2.24 |
| 443787 | AV646505 | Hs. 122155 | ESTs | 5.12E−04 | 2.24 |
| 427581 | NM_014788 | Hs. 179703 | KIAA0129 gene product | 1.27E−10 | 2.23 |
| 416224 | NM_002902 | Hs. 79088 | reticulocalbin 2, EF-hand calcium bindin | 1.37E−07 | 2.23 |
| 405533 | | | Target Exon | 1.20E−04 | 2.23 |
| 402230 | | | Fgenesh predicted: CYTOCHROME P450 4F5 ( | 2.46E−06 | 2.23 |
| 429857 | AF089897 | Hs. 294030 | topoisomerase-related function protein 4 | 5.01E−06 | 2.21 |
| 437536 | X91221 | Hs. 144465 | ESTs | 1.55E−05 | 2.21 |
| 446094 | AK001760 | Hs. 13801 | KIAA1685 protein | 1.54E−07 | 2.20 |
| 444097 | AW517412 | Hs. 150757 | ESTs | 3.40E−03 | 2.20 |
| 425268 | AI807883 | Hs. 180059 | Homo sapiens cDNA FLJ20653 fis, clone KA | 3.58E−04 | 2.20 |
| 400751 | | | NM_003105*: Homo sapiens sortilin-related | 4.17E−04 | 2.20 |
| 424145 | AW802763 | Hs. 193124 | pyruvate dehydrogenase kinase, isoenzyme | 2.84E−06 | 2.19 |
| 430339 | W28608 | Hs. 239625 | integral membrane protein 2B | 2.63E−06 | 2.19 |
| 419287 | X91906 | Hs. 89872 | chloride channel 5 (nephrolithiasis 2, X | 1.23E−06 | 2.19 |
| 427374 | AI150033 | Hs. 143686 | ESTs | 8.34E−03 | 2.19 |
| 427043 | AA397679 | | ESTs | 2.93E−04 | 2.19 |
| 400694 | | | Target Exon | 5.84E−07 | 2.18 |
| 421014 | NM_012227 | | Pseudoautosomal GTP-binding protein-like | 6.96E−03 | 2.17 |
| 408078 | N50928 | Hs. 45203 | ESTs | 8.51E−04 | 2.17 |
| 447009 | AB002340 | Hs. 16950 | KIAA0342 gene product | 8.64E−07 | 2.17 |
| 422239 | AI878922 | Hs. 180139 | SMT3 (suppressor of mif two 3. yeast) ho | 6.16E−07 | 2.16 |
| 413786 | AW613780 | Hs. 13500 | ESTs | 6.01E−10 | 2.16 |
| 438307 | AB011093 | Hs. 6150 | hypothetical protein MGC15913 | 1.91E−08 | 2.16 |
| 455187 | AW864050 | | gb: PM2-SN0013-120400-003-c12 SN0013 Homo | 1.69E−03 | 2.15 |
| 459487 | AA699665 | | gb: zi78b05.s1 Soares_fetal_liver_spleen_{—} | 7.20E−04 | 2.15 |
| 449935 | AA004798 | Hs. 108311 | ESTs, Weakly similar to T00351 hypotheti | 9.75E−09 | 2.15 |
| 428695 | AI355647 | Hs. 189999 | purinergic receptor (family A group 5) | 1.45E−06 | 2.15 |
| 449964 | AW001741 | Hs. 24243 | hypothetical protein FLJ10706 | 1.23E−04 | 2.14 |
| 427398 | AW390020 | Hs. 20415 | chromosome 21 open reading frame 11 | 3.46E−04 | 2.14 |
| 440548 | AL117408 | Hs. 7274 | DKFZP434P1750 protein | 3.31E−03 | 2.14 |
| 440464 | AI917706 | Hs. 129997 | ESTs | 1.49E−05 | 2.14 |
| 422515 | AW500470 | Hs. 117950 | multifunctional polypeptide similar to S | 1.55E−05 | 2.14 |
| 451190 | H38361 | Hs. 26045 | protein tyrosine phosphatase, receptor t | 2.99E−05 | 2.13 |
| 422649 | BE258692 | Hs. 118910 | tumor susceptibility gene 101 | 1.82E−06 | 2.13 |
| 435374 | AI049889 | | ESTs | 4.37E−04 | 2.13 |
| 423062 | NM_003655 | Hs. 5637 | ESTs | 8.76E−07 | 2.13 |
| 425164 | R18189 | | gb: yf98f10.r1 Soares infant brain 1NIB H | 4.67E−04 | 2.12 |
| 421685 | AF189723 | Hs. 106778 | ATPase, Ca transporting, type 2C, member | 6.05E−05 | 2.12 |
| 440266 | AA088809 | Hs. 19525 | hypothetical protein FLJ22794 | 5.23E−06 | 2.12 |
| 443574 | U83993 | Hs. 321709 | purinergic receptor P2X, ligand-gated io | 7.69E−05 | 2.12 |
| 447735 | AA775268 | Hs. 6127 | Homo sapiens cDNA: FLJ23020 fis, clone L | 3.38E−04 | 2.11 |
| 433426 | H69125 | Hs. 133525 | ESTs | 1.40E−07 | 2.11 |
| 450056 | BE047394 | Hs. 8208 | ESTs, Weakly similar to S71512 hypotheti | 3.36E−04 | 2.11 |
| 418027 | AB037807 | Hs. 83293 | hypothetical protein | 1.30E−06 | 2.11 |
| 421650 | AA781795 | Hs. 122587 | ESTs | 2.45E−07 | 2.11 |
| 453909 | AW004045 | Hs. 203365 | ESTs | 2.51E−04 | 2.11 |
| 444847 | AI199345 | Hs. 153393 | ESTs | 1.43E−04 | 2.11 |
| 416448 | L13210 | Hs. 79339 | lectin, galactoside-binding, soluble, 3 | 3.05E−11 | 2.10 |
| 449140 | AW013840 | Hs. 202092 | ESTs | 6.30E−05 | 2.10 |
| 430593 | AA985190 | Hs. 246875 | hypothetical protein FLJ20059 | 5.90E−04 | 2.10 |
| 408791 | AW272251 | Hs. 254651 | ESTs | 1.02E−04 | 2.10 |
| 416435 | AI431301 | | KIAA0129 gene product | 4.53E−07 | 2.10 |
| 431637 | AI879330 | Hs. 265960 | hypothetical protein FLJ10563 | 9.82E−04 | 2.10 |
| 442547 | AA306997 | Hs. 217484 | ESTs, Weakly similar to ALU1_HUMAN ALU S | 3.06E−04 | 2.10 |
| 419560 | AA456130 | | KIAA1255 protein | 7.10E−05 | 2.10 |
| 441673 | AI018657 | | ESTs | 1.80E−04 | 2.09 |
| 452827 | AI571835 | Hs. 55468 | ESTs | 9.60E−07 | 2.09 |
| 430341 | NM_006348 | Hs. 239631 | golgi transport complex 1 (90 kDa subuni | 2.32E−04 | 2.09 |
| 442160 | AI337127 | Hs. 156325 | ESTs | 6.80E−09 | 2.09 |
| 416754 | H07145 | Hs. 6799 | ESTs, Weakly similar to T12483 hypotheti | 1.55E−05 | 2.09 |
| 446731 | BE378875 | Hs. 16059 | HSPC009 protein | 1.50E−03 | 2.09 |
| 434128 | W93170 | Hs. 284164 | protein x 0004 | 2.58E−06 | 2.08 |
| 426801 | AA486846 | Hs. 271795 | ESTs, Weakly similar to I38022 hypotheti | 4.92E−05 | 2.08 |
| 412556 | AW961963 | | gb: EST374036 MAGE resequences, MAGG Homo | 4.04E−03 | 2.08 |
| 401467 | | | NM_022137*: Homo sapiens secreted modular | 8.05E−03 | 2.08 |
| 416714 | AF283770 | Hs. 79630 | CD79A antigen (immunoglobulin-associated | 2.38E−03 | 2.08 |
| 441297 | AW403084 | Hs. 7766 | ubiquitin-conjugating enzyme E2E 1 (homo | 4.09E−05 | 2.08 |
| 449689 | AF228421 | Hs. 23889 | DKFZP564A032 protein | 1.72E−04 | 2.07 |
| 432360 | BE045243 | Hs. 274416 | Target CAT | 4.08E−07 | 2.07 |
| 424308 | AW975531 | Hs. 154443 | minichromosome maintenance deficient (S. | 2.18E−07 | 2.07 |
| 424618 | L29472 | Hs. 1802 | major histocompatibility complex, class | 3.53E−06 | 2.07 |
| 421261 | AA600853 | Hs. 98133 | ESTs | 1.01E−03 | 2.06 |
| 430200 | BE613337 | Hs. 234896 | geminin | 1.10E−11 | 2.06 |
| 423968 | AF098277 | Hs. 136529 | solute carrier family 23 (nucleobase tra | 1.34E−07 | 2.06 |
| 453313 | BE005771 | Hs. 153746 | hypothetical protein FLJ22490 | 3.19E−03 | 2.06 |
| 403789 | | | NM_001334*: Homo-sapiens cathepsin O (CTS | 4.24E−05 | 2.06 |
| 427561 | AI123333 | Hs. 134191 | ESTs | 1.07E−08 | 2.06 |
| 454877 | AW836463 | | gb: PM3-LT0032-231299-001-d01 LT0032 Homo | 6.46E−07 | 2.06 |
| 408716 | AI567839 | Hs. 151714 | Homo sapiens mRNA for KIAA1769 protein, | 7.10E−07 | 2.06 |
| 438182 | AW342140 | Hs. 182545 | ESTs, Weakly similar to ALU1_HUMAN ALU S | 3.05E−05 | 2.05 |
| 441993 | BE395143 | Hs. 71523 | ESTs, Weakly similar to S44243 endosomal | 7.66E−04 | 2.05 |
| 427648 | AI376722 | Hs. 180062 | proteasome (prosome, macropain) subunit, | 6.20E−10 | 2.05 |
| 414646 | AA353776 | Hs. 901 | CD48 antigen (B-cell membrane protein) | 4.38E−07 | 2.05 |
| 458079 | AI796870 | Hs. 54277 | DNA segment on chromosome X (unique) 992 | 1.32E−05 | 2.05 |
| 446783 | AW138343 | Hs. 141867 | ESTs | 3.84E−03 | 2.05 |
| 406117 | | | C19001795: gi\|10434258\|dbj\|BAB14191.1\|(A | 3.18E−06 | 2.05 |
| 453255 | AA278167 | Hs. 19215 | Homo sapiens, clone IMAGE: 3605822, mRNA | 3.20E−04 | 2.04 |
| 446619 | AU076643 | Hs. 313 | secreted phosphoprotein 1 (osteopontin, | 9.85E−03 | 2.04 |
| 437967 | BE277414 | Hs. 5947 | mel transforming oncogene (derived from | 1.65E−04 | 2.04 |
| 400137 | | | Eos Control | 1.05E−07 | 2.04 |
| 417787 | R14948 | Hs. 23883 | ESTs | 9.00E−07 | 2.04 |
| 417600 | R01279 | Hs. 16179 | hypothetical protein FLJ23467 | 8.47E−03 | 2.04 |
| 411562 | AL050201 | Hs. 70769 | hypothetical protein DKFZp586E1923 | 1.33E−06 | 2.04 |
| 418733 | AA227714 | | KIAA0129 gene product | 1.31E−09 | 2.03 |
| 412651 | AA115333 | Hs. 107968 | ESTs | 6.35E−09 | 2.03 |
| 406420 | | | C19000229*: gi\|6753826\|ref\|NP_034311.1\|f | 1.99E−03 | 2.03 |
| 423397 | NM_001838 | Hs. 1652 | chemokine (C-C motif) receptor 7 | 4.05E−05 | 2.03 |
| 447002 | BE242866 | Hs. 16933 | HepA-related protein | 1.96E−06 | 2.03 |
| 409038 | T97490 | Hs. 50002 | small inducible cytokine subfamily A (Cy | 4.88E−04 | 2.03 |
| 419737 | H24185 | Hs. 92918 | hypothetical protein | 2.46E−04 | 2.03 |
| 436235 | AI084982 | Hs. 120790 | ESTs | 4.48E−05 | 2.03 |
| 447043 | BE410773 | Hs. 115412 | hypothetical protein FLJ13881 | 3.32E−06 | 2.02 |
| 411987 | AA375975 | Hs. 24831 | ESTs, Moderately similar to ALU8_HUMAN A | 5.80E−04 | 2.02 |
| 400245 | | | Eos Control | 1.28E−06 | 2.02 |
| 447906 | AL050062 | Hs. 19999 | DKFZP566K023 protein | 5.25E−05 | 2.02 |
| 451820 | AW058357 | Hs. 199248 | ESTs | 2.88E−05 | 2.02 |
| 418216 | AA662240 | Hs. 283099 | AF15q14 protein | 5.20E−10 | 2.02 |
| 408949 | AF189011 | Hs. 49163 | putative ribonuclease III | 2.94E−05 | 2.01 |
| 423962 | R73257 | Hs. 42586 | KIAA1560 protein | 2.03E−04 | 2.01 |
| 432053 | AI024572 | | ESTs | 8.07E−08 | 2.01 |
| 412754 | AW160375 | Hs. 74565 | amyloid beta (A4) precursor-like protein | 1.10E−03 | 2.01 |
| 453927 | AA082465 | Hs. 125031 | choline/ethanolaminephosphotransferase | 6.17E−08 | 2.01 |
| 430556 | AW967807 | Hs. 13797 | ESTs | 3.75E−04 | 2.01 |
| 451494 | AI799444 | Hs. 247095 | ESTs, Moderately similar to ALU7_HUMAN A | 9.08E−04 | 2.00 |
| 422546 | AB007969 | Hs. 301478 | KIAA0500 protein | 1.62E−06 | 2.00 |
| 424755 | AB033094 | Hs. 152925 | KIAA1268 protein | 5.02E−08 | 2.00 |
| 406782 | AA430373 | | gb: zw20f11.s1 Soares ovary tumor NbHOT H | 2.29E−03 | 1.99 |
| 427128 | AW301984 | Hs. 49349 | hypothetical protein FLJ12619 | 1.86E−07 | 1.99 |
| 414159 | AW511414 | Hs. 257352 | apolipoprotein L, 6 | 5.86E−12 | 1.99 |
| 418348 | AI537167 | Hs. 96322 | hypothetical protein FLJ23560 | 6.15E−04 | 1.99 |
| 410369 | AW975159 | Hs. 293097 | ESTs, Weakly similar to A55380 faciogeni | 5.39E−05 | 1.99 |
| 410315 | AI638871 | Hs. 152519 | Homo sapiens cDNA: FLJ22524 fis, clone H | 4.48E−07 | 1.99 |
| 407644 | D16815 | | nuclear receptor subfamily 1, group D, m | 1.10E−05 | 1.98 |
| 459107 | AA811881 | Hs. 28505 | ubiquitin-conjugating enzyme E2H (homolo | 1.49E−03 | 1.98 |
| 433150 | AB035274 | Hs. 21320 | postreplication repair protein hRAD18p | 8.38E−04 | 1.98 |
| 413823 | AI341417 | Hs. 29406 | ESTs | 9.53E−08 | 1.97 |
| 409518 | BE384836 | Hs. 3454 | KIAA1821 protein | 2.52E−04 | 1.97 |
| 439225 | AA192669 | Hs. 45032 | ESTs | 1.00E−06 | 1.97 |
| 430619 | AF129756 | Hs. 247452 | HLA-B associated transcript-5 | 1.86E−06 | 1.97 |
| 432606 | NM_002104 | Hs. 3066 | granzyme K (serine protease, granzyme 3; | 1.05E−06 | 1.97 |
| 441298 | AW136267 | Hs. 166629 | ESTs | 6.99E−03 | 1.96 |
| 450931 | N25156 | Hs. 25648 | tumor necrosis factor receptor superfami | 4.52E−08 | 1.96 |
| 457234 | AW968360 | Hs. 14355 | Homo sapiens cDNA FLJ13207 fis, clone NT | 1.08E−04 | 1.96 |
| 442328 | AI952430 | Hs. 150614 | ESTs, Weakly similar to ALU4_HUMAN ALU S | 1.07E−04 | 1.96 |
| 414493 | AL133921 | Hs. 76272 | retinoblastoma-binding protein 2 | 1.41E−07 | 1.96 |
| 427209 | H06509 | Hs. 92423 | KIAA1566 protein | 1.67E−06 | 1.96 |
| 416430 | H60487 | Hs. 159440 | bile acid Coenzyme A: amino acid N-acylt | 1.43E−05 | 1.96 |
| 448692 | AW013907 | Hs. 167531 | methylcrotonoyl-Coenzyme A carboxylase 2 | 2.14E−03 | 1.95 |
| 418707 | U97502 | Hs. 87497 | butyrophilin, subfamily 3, member A2 | 2.72E−11 | 1.95 |
| 440256 | U23841 | Hs. 18851 | hypothetical protein FLJ10875 | 5.24E−06 | 1.95 |
| 436578 | AI091435 | Hs. 134859 | ESTs | 1.08E−06 | 1.94 |
| 423597 | AL043117 | Hs. 129872 | sperm associated antigen 9 | 2.72E−04 | 1.94 |
| 446669 | AW972832 | Hs. 29468 | ESTs | 1.10E−04 | 1.93 |
| 436061 | AI248584 | Hs. 190745 | Homo sapiens cDNA: FLJ21326 fis, clone C | 3.75E−03 | 1.93 |
| 408291 | AB023191 | Hs. 44131 | KIAA0974 protein | 4.99E−05 | 1.93 |
| 432485 | N90866 | Hs. 276770 | CDW52 antigen (CAMPATH-1 antigen) | 5.39E−07 | 1.93 |
| 420653 | AI224532 | Hs. 88550 | ESTs | 6.18E−03 | 1.93 |
| 418504 | BE159718 | Hs. 85335 | Homo sapiens mRNA; cDNA DKFZp564D1462 (f | 1.63E−06 | 1.93 |
| 444398 | AI146775 | Hs. 143550 | ESTs | 7.61E−03 | 1.93 |
| 408731 | R85652 | | Homo sapiens mRNA; cDNA DKFZp434F1928 (f | 3.52E−04 | 1.93 |
| 436395 | AJ227900 | | gb: Homo sapiens partial mRNA; ID EE2-16B | 4.64E−08 | 1.92 |
| 429276 | AF056085 | Hs. 198612 | G protein-coupled receptor 51 | 8.50E−05 | 1.92 |
| 426476 | NM_003296 | Hs. 2042 | testis specific protein 1 (probe H4-1 p3 | 1.20E−03 | 1.92 |
| 454075 | R43826 | Hs. 16313 | Kruppel-like zinc finger protein GLIS2 | 7.10E−03 | 1.92 |
| 445245 | AB032973 | Hs. 12461 | LCHN protein | 2.74E−10 | 1.91 |
| 457665 | BE551196 | Hs. 114275 | ESTs | 5.71E−03 | 1.91 |
| 418090 | U57059 | Hs. 83429 | tumor necrosis factor (ligand) superfami | 1.34E−09 | 1.91 |
| 427675 | AW138190 | Hs. 180248 | zinc finger protein 124 (HZF-16) | 3.00E−06 | 1.91 |
| 435745 | AW967059 | | Homo sapiens clone 24711 mRNA sequence | 5.05E−04 | 1.91 |
| 429798 | AL117578 | Hs. 222909 | DKFZP434C128 protein | 5.79E−09 | 1.91 |
| 424637 | NM_015057 | Hs. 151411 | KIAA0916 protein | 7.41E−07 | 1.91 |
| 445447 | H14522 | | ribosomal protein S4, X-linked | 4.52E−05 | 1.91 |
| 428372 | AK000684 | Hs. 183887 | hypothetical protein FLJ22104 | 4.09E−08 | 1.90 |
| 444454 | BE018316 | Hs. 11183 | sorting nexin 2 | 1.42E−06 | 1.90 |
| 431122 | AI267593 | Hs. 250535 | Homo sapiens mRNA; cDNA DKFZp434N2412 (f | 2.54E−04 | 1.90 |
| 436318 | T77359 | Hs. 14040 | Homo sapiens cDNA: FLJ21772 fis, clone C | 4.82E−04 | 1.90 |
| 446553 | AB021179 | Hs. 15299 | HMBA-inducible | 6.90E−08 | 1.90 |
| 433401 | AF039698 | Hs. 284217 | serologically defined colon cancer antig | 6.31E−08 | 1.89 |
| 421677 | H64092 | Hs. 38282 | ESTs | 2.70E−08 | 1.89 |
| 424194 | BE245833 | Hs. 169854 | gb: TCBAP1E1908 Pedialric pre-B cell acut | 2.67E−03 | 1.89 |
| 426990 | AL044315 | Hs. 173094 | Homo sapiens mRNA for KIAA1750 protein. | 2.18E−04 | 1.89 |
| 439810 | AL109710 | Hs. 85568 | EST | 1.53E−03 | 1.89 |
| 414757 | U46922 | Hs. 77252 | fragile histidine triad gene | 6.95E−06 | 1.89 |
| 430587 | AK000341 | Hs. 246107 | elongation of very long chain fatty acid | 1.22E−05 | 1.88 |
| 440527 | AV657117 | Hs. 184164 | ESTs, Moderately similar to S65657 alpha | 2.16E−06 | 1.88 |
| 414405 | AI362533 | | KIAA0306 protein | 1.88E−06 | 1.88 |
| 414171 | AA360328 | Hs. 865 | RAP1 A, member of RAS oncogene family | 1.72E−04 | 1.88 |
| 448198 | BE622100 | Hs. 209406 | ESTs, Weakly similar to I38600 zinc fing | 7.63E−07 | 1.88 |
| 433017 | Y15067 | Hs. 279914 | zinc finger protein 232 | 1.66E−06 | 1.88 |
| 449609 | BE246434 | Hs. 289026 | guanine nucleotide binding protein (G pr | 4.83E−05 | 1.88 |
| 427600 | AW630918 | Hs. 179774 | proteasome (prosome, macropain) activato | 1.19E−06 | 1.88 |
| 409362 | AC008044 | Hs. 54418 | alkylation repair; alkB homolog | 6.66E−03 | 1.88 |
| 450379 | T77813 | Hs. 268590 | ESTs | 4.05E−04 | 1.87 |
| 404426 | | | C8000068*: gi\|5453579\|ref\|NP_006120.1\| bo | 1.01E−08 | 1.87 |
| 407949 | W21874 | Hs. 247057 | ESTs, Weakly similar to 2109260A B cell | 9.97E−06 | 1.87 |
| 446570 | AV659177 | Hs. 127160 | ESTs | 4.44E−07 | 1.87 |
| 409896 | AW205479 | Hs. 279780 | NY-REN-18 antigen | 2.36E−08 | 1.87 |
| 419867 | NM_014246 | Hs. 252387 | cadherin, EGF LAG seven-pass G-type rece | 3.90E−04 | 1.87 |
| 447178 | AW594641 | Hs. 192417 | ESTs | 5.76E−07 | 1.87 |
| 438475 | W03856 | Hs. 13188 | ESTs, Highly similar to Gene product wit | 6.54E−09 | 1.87 |
| 443492 | AI073989 | Hs. 144913 | hypothetical protein FLJ23514 | 3.68E−04 | 1.87 |
| 400303 | AA242758 | Hs. 79136 | LIV-1 protein, estrogen regulated | 1.02E−09 | 1.87 |
| 417052 | NM_000712 | Hs. 81029 | biliverdin reductase A | 1.44E−08 | 1.87 |
| 452406 | AI002968 | Hs. 235402 | ESTs, Weakly similar to T26525 hypotheti | 5.04E−08 | 1.86 |
| 432857 | NM_016103 | Hs. 279582 | GTP-binding protein Sara | 4.59E−07 | 1.86 |
| 428933 | AA743433 | Hs. 119292 | ESTs | 1.97E−04 | 1.86 |
| 429360 | AK000658 | Hs. 200332 | hypothetical protein FLJ20651 | 2.52E−06 | 1.86 |
| 422352 | AA766296 | Hs. 99200 | ESTs | 6.40E−05 | 1.86 |
| 438449 | AK001333 | Hs. 6216 | Homo sapiens hepatocellular carcinoma-as | 5.96E−04 | 1.86 |
| 421524 | AA312082 | Hs. 105445 | GDNF family receptor alpha 1 | 5.48E−08 | 1.86 |
| 419598 | AW410557 | Hs. 91471 | ATPase, Class II, type 9B | 1.54E−03 | 1.86 |
| 445317 | AI219856 | | ESTs | 2.25E−04 | 1.86 |
| 401274 | | | Target Exon | 9.48E−05 | 1.86 |
| 433028 | AI199144 | Hs. 283737 | AD-017 protein | 2.06E−05 | 1.86 |
| 419439 | BE252908 | | thyroglobulin | 2.28E−06 | 1.86 |
| 453037 | AA045175 | Hs. 17914 | ESTs | 8.76E−07 | 1.86 |
| 432468 | AW402155 | Hs. 3003 | CD3E antigen, epsilon polypeptide (TiT3 | 2.04E−03 | 1.86 |
| 450997 | AW580830 | Hs. 35254 | hypothetical protein FLB6421 | 8.02E−10 | 1.86 |
| 441787 | AA813278 | Hs. 9788 | hypothetical protein MGC10924 similar to | 1.19E−06 | 1.86 |
| 450381 | AW291767 | Hs. 189829 | hypothetical protein FLJ12765 | 1.37E−03 | 1.86 |
| 410199 | AW377424 | Hs. 205126 | Homo sapiens cDNA: FLJ22667 fis, clone H | 2.36E−08 | 1.86 |
| 423215 | C16770 | | gb: C16770 Clontech human aorta polyA mRN | 8.56E−03 | 1.85 |
| 422085 | AB018257 | Hs. 288773 | zinc finger protein 294 | 1.80E−04 | 1.85 |
| 429358 | AB037825 | Hs. 200317 | KIAA1404 protein | 1.28E−08 | 1.85 |
| 447631 | AW140016 | Hs. 161415 | ESTs | 4.33E−06 | 1.85 |
| 453954 | AW118336 | Hs. 75251 | DEAD/H (Asp-Glu-Ala-Asp/His) box binding | 6.32E−04 | 1.85 |
| 438050 | BE262816 | Hs. 6061 | protein kinase, AMP-activated, beta 1 no | 6.39E−04 | 1.85 |
| 425939 | AK001527 | Hs. 163953 | hypothetical protein FLJ10665 | 2.37E−03 | 1.85 |
| 426911 | AA812691 | Hs. 278985 | ESTs | 1.44E−04 | 1.85 |
| 426126 | AL118747 | Hs. 26691 | ESTs | 7.85E−05 | 1.85 |
| 400929 | | | ENSP00000252232*: Sterol regulatory eleme | 1.42E−03 | 1.85 |
| 413048 | M93221 | Hs. 75182 | mannose receptor, C type 1 | 6.72E−07 | 1.85 |
| 420613 | AI873871 | Hs. 7041 | ESTs, Weakly similar to A47582 B-cell gr | 5.30E−09 | 1.85 |
| 409533 | AW969543 | Hs. 21291 | mitogen-activated protein kinase kinase | 2.39E−04 | 1.85 |
| 430024 | AI808780 | Hs. 227730 | integrin, alpha 6 | 5.50E−07 | 1.85 |
| 407110 | AA018042 | Hs. 252085 | Prader-Willi/Angelman syndrome-5 | 1.23E−05 | 1.85 |
| 403790 | | | NM_001334*: Homo sapiens cathepsin O (CTS | 1.67E−05 | 1.84 |
| 408214 | AL120445 | Hs. 77823 | hypothetical protein FLJ21343 | 9.69E−06 | 1.84 |
| 443285 | AI301918 | Hs. 334264 | ESTs | 7.83E−04 | 1.84 |
| 445615 | AW449905 | Hs. 40919 | Homo sapiens cDNA FLJ14511 fis. clone NT | 321E−04 | 1.84 |
| 449720 | AA311152 | Hs. 288708 | hypothetical protein FLJ21562 | 4.25E−06 | 1.84 |
| 445806 | AL137516 | Hs. 13323 | hypothetical protein FLJ22059 | 1.57E−04 | 1.84 |
| 457400 | AF032906 | Hs. 252549 | cathepsin Z | 1.05E−07 | 1.84 |
| 424571 | BE379766 | | polymerase (RNA) II (DNA directed) polyp | 1.04E−10 | 1.84 |
| 408328 | R93852 | Hs. 28411 | ESTs | 5.70E−04 | 1.84 |
| 436363 | AA843926 | Hs. 124434 | ESTs | 6.44E−05 | 1.84 |
| 418850 | AJ242977 | Hs. 89278 | hypothetical protein FLJ11186 | 4.88E−09 | 1.83 |
| 422711 | D60641 | | Homo sapiens mRNA; cDNA DKFZp586l1518 (f | 3.71E−05 | 1.83 |
| 444889 | AI917770 | | ESTs | 6.36E−05 | 1.83 |
| 446506 | AI123118 | Hs. 15159 | chemokine-like factor, alternatively spl | 3.56E−11 | 1.83 |
| 424437 | BE244700 | Hs. 147049 | cut (Drosophila)-like 1 (CCAAT displacem | 1.49E−04 | 1.83 |
| 422371 | NM_001882 | Hs. 115617 | corticotropin releasing hormone-binding | 2.26E−07 | 1.83 |
| 419461 | AI452601 | Hs. 288869 | nuclear receptor subfamily 2, group F, m | 5.42E−09 | 1.83 |
| 434629 | AA789081 | Hs. 4029 | glioma-amplified sequence-41 | 1.80E−05 | 1.83 |
| 425367 | BE271188 | Hs. 155975 | protein tyrosine phosphatase, receptor t | 1.53E−05 | 1.82 |
| 453275 | AW247236 | Hs. 32826 | CGI-130 protein | 1.86E−07 | 1.82 |
| 418751 | BE389014 | | phosphoinositide-3-kinase, regulatory su | 1.59E−07 | 1.82 |
| 445525 | BE149866 | Hs. 14831 | Homo sapiens, Similar to zinc linger pro | 5.85E−07 | 1.82 |
| 432107 | AA779945 | Hs. 303294 | ESTs, Weakly similar to ALUB_HUMAN !!!! | 3.18E−03 | 1.82 |
| 407355 | AA846203 | Hs. 193974 | ESTs, Weakly similar to ALU1_HUMAN ALU S | 5.23E−04 | 1.82 |
| 422646 | H87863 | Hs. 151380 | ESTs, Weakly similar to T16584 hypotheti | 1.30E−04 | 1.82 |
| 414485 | W27026 | Hs. 182625 | VAMP (vesicle-associated membrane protei | 4.19E−03 | 1.82 |
| 421931 | NM_000814 | Hs. 1440 | gamma-aminobutyric acid (GABA) A recepto | 9.00E−05 | 1.82 |
| 454056 | AI368836 | Hs. 24808 | ESTs, Weakly similar to I38022 hypotheti | 2.84E−04 | 1.82 |
| 459067 | AW207623 | Hs. 262716 | ESTs, Moderately similar to S65657 alpha | 1.04E−03 | 1.81 |
| 422530 | AW972300 | Hs. 118110 | bone marrow stromal cell antigen 2 | 5.31E−11 | 1.81 |
| 432051 | AF112461 | Hs. 272383 | G protein-coupled receptor 57 | 2.98E−03 | 1.81 |
| 405268 | | | ENSP00000223174*: KIAA0783 PROTEIN. | 7.25E−05 | 1.81 |
| 419951 | AI653415 | Hs. 195789 | ESTs | 3.51E−06 | 1.81 |
| 457259 | AA459713 | Hs. 36475 | KIAA0493 protein | 1.47E−04 | 1.81 |
| 441683 | BE564214 | Hs. 102946 | ESTs | 4.30E−07 | 1.81 |
| 421263 | AB020638 | Hs. 103000 | KIAA0831 protein | 4.10E−08 | 1.80 |
| 431696 | AA259068 | Hs. 267819 | protein phosphatase 1, regulatory (inhib | 2.00E−03 | 1.80 |
| 437143 | AW204056 | Hs. 8917 | ESTs | 2.07E−09 | 1.80 |
| 450728 | AW162923 | Hs. 25363 | presenilin 2 (Alzheimer disease 4) | 5.41E−05 | 1.80 |
| 417312 | AW888411 | Hs. 250811 | leukemia-associated phosphoprotein p18 ( | 3.81E−06 | 1.80 |
| 432647 | AI807481 | Hs. 278581 | fibroblast growth factor receptor 2 (bac | 8.16E−07 | 1.80 |
| 424232 | AB015982 | Hs. 143460 | protein kinase C, nu | 1.49E−04 | 1.80 |
| 412715 | NM_000947 | Hs. 74519 | primase, polypeptide 2A (58 kD) | 8.01E−05 | 1.80 |
| 450260 | AW090384 | Hs. 7739 | hypothetical protein MGC12904 | 1.06E−04 | 1.80 |
| 424862 | AB011472 | Hs. 153546 | CDC23 (cell division cycle 23, yeast, ho | 9.20E−08 | 1.80 |
| 430060 | NM_002941 | Hs. 301198 | roundabout (axon guidance receptor, Dros | 4.39E−03 | 1.80 |
| 420338 | AA825595 | Hs. 88269 | Homo sapiens, clone MGC: 17339, mRNA, com | 8.17E−07 | 1.80 |
| 402855 | | | NM_001839*: Homo sapiens calponin 3, acid | 8.44E−04 | 1.80 |
| 430043 | AI040009 | Hs. 306327 | Homo sapiens mRNA; cDNA DKFZp434A012 (fr | 4.12E−07 | 1.79 |
| 438122 | AI620270 | Hs. 129837 | ESTs, Weakly similar to Z263_HUMAN ZINC | 1.45E−03 | 1.79 |
| 432460 | H12912 | Hs. 274691 | adenylate kinase 3 | 4.12E−10 | 1.79 |
| 445523 | Z30118 | Hs. 293788 | ESTs, Moderately similar to unnamed prot | 7.17E−05 | 1.79 |
| 406765 | AA609132 | Hs. 76662 | hypothetical protein MGC2993 | 9.48E−08 | 1.79 |
| 400517 | | | lengsin | 4.89E−07 | 1.79 |
| 421804 | AF032105 | Hs. 108447 | spinocerebellar ataxia 7 (olivopontocere | 2.48E−05 | 1.79 |
| 424653 | AW977534 | Hs. 151469 | calcium/calmodulin-dependent serine prot | 8.73E−04 | 1.79 |
| 418240 | BE019261 | Hs. 83869 | hypothetical protein | 1.76E−05 | 1.79 |
| 458196 | AI802408 | | ubiquitin A-52 residue ribosomal protein | 1.02E−03 | 1.79 |
| 433528 | AK001605 | Hs. 3376 | hypothetical protein FLJ10743 | 2.88E−05 | 1.79 |
| 402976 | | | Target Exon | 3.60E−04 | 1.78 |
| 433730 | AK002135 | Hs. 3542 | hypothetical protein FLJ11273 | 6.69E−06 | 1.78 |
| 446874 | AW968304 | Hs. 56156 | ESTs | 1.09E−05 | 1.78 |
| 436856 | AI469355 | Hs. 127310 | ESTs | 5.43E−09 | 1.78 |
| 459724 | AI732889 | Hs. 47341 | ESTs, Weakly similar to alternatively sp | 2.77E−03 | 1.78 |
| 433707 | AI791584 | Hs. 130741 | ESTs | 3.51E−03 | 1.78 |
| 449523 | NM_000579 | Hs. 54443 | chemokine (C-C motif) receptor 5 | 3.38E−06 | 1.78 |
| 453250 | AI346520 | Hs. 121619 | chromosome 11 open reading frame 15 | 2.30E−08 | 1.77 |
| 430718 | AI792814 | | Homo sapiens BAC clone RP11-335J18 from | 8.53E−04 | 1.77 |
| 452065 | AK000360 | Hs. 27721 | Wolf-Hirschhorn syndrome candidate 1-lik | 8.41E−04 | 1.77 |
| 426899 | AL043221 | Hs. 172825 | KIAA1037 protein | 9.25E−07 | 1.77 |
| 441791 | AW372449 | Hs. 61271 | hypothetical protein FLJ21159 | 1.05E−04 | 1.77 |
| 413029 | AL119399 | Hs. 293850 | ESTs | 7.99E−05 | 1.76 |
| 453319 | AI985369 | Hs. 301134 | ESTs | 1.19E−03 | 1.76 |
| 457425 | AW139202 | Hs. 301209 | myeloid/lymphoid or mixed-lineage leukem | 5.83E−05 | 1.76 |
| 445786 | AW629819 | Hs. 144502 | hypothetical protein FLJ22055 | 3.11E−06 | 1.76 |
| 419175 | AW270037 | | KIAA0779 protein | 7.55E−08 | 1.76 |
| 428291 | AA534009 | Hs. 183487 | interferon stimulated gene (20 kD) | 9.79E−06 | 1.76 |
| 408405 | AK001332 | Hs. 44672 | hypothetical protein FLJ10470 | 2.98E−11 | 1.76 |
| 426310 | NM_000909 | Hs. 169266 | neuropeptide Y receptor Y1 | 1.50E−03 | 1.75 |
| 414812 | X72755 | Hs. 77367 | monokine induced by gamma interferon | 5.22E−06 | 1.75 |
| 453020 | AL162039 | Hs. 31422 | Homo sapiens mRNA; cDNA DKFZp434M229 (fr | 2.69E−04 | 1.75 |
| 403743 | | | C1002604: gi\|8393668\|ref\|NP_058989.1\| kin | 1.47E−04 | 1.75 |
| 433675 | AW977653 | Hs. 75319 | ribonucleotide reductase M2 polypeptide | 7.35E−05 | 1.75 |
| 410691 | AW239226 | Hs. 65450 | reticulon 4 | 3.22E−06 | 1.75 |
| 421443 | BE550141 | Hs. 156148 | hypothetical protein FLJ13231 | 9.38E−05 | 1.74 |
| 414183 | AW957446 | Hs. 301711 | ESTs | 1.83E−04 | 1.74 |
| 427221 | L15409 | Hs. 174007 | von Hippel-Lindau syndrome | 3.24E−04 | 1.74 |
| 443589 | H52931 | Hs. 165067 | ESTs | 3.32E−05 | 1.74 |
| 449480 | AI741617 | Hs. 108447 | spinocerebellar ataxia 7 (olivopontocere | 1.75E−05 | 1.74 |
| 442272 | AA988302 | Hs. 129172 | ESTs | 2.85E−03 | 1.74 |
| 429747 | M87507 | Hs. 2490 | caspase 1, apoptosis-related cysteine pr | 2.73E−07 | 1.74 |
| 414261 | BE267466 | Hs. 13561 | hypothetical protein MGC4692 | 9.05E−03 | 1.74 |
| 442138 | AA445973 | Hs. 13303 | Homo sapiens cDNA: FLJ21784 fis, clone H | 3.64E−05 | 1.74 |
| 412584 | X54870 | Hs. 74085 | DNA segment on chromosome 12 (unique) 24 | 1.90E−06 | 1.74 |
| 406825 | AI982529 | Hs. 84298 | CD74 antigen (invariant polypeptide of m | 9.75E−06 | 1.73 |
| 421893 | NM_001078 | Hs. 109225 | vascular cell adhesion molecule 1 | 6.57E−07 | 1.73 |
| 432094 | AI658580 | Hs. 61426 | Homo sapiens mesenchymal stem cell prote | 7.20E−07 | 1.73 |
| 420344 | BE463721 | Hs. 97101 | putative G protein-coupled receptor | 9.18E−04 | 1.73 |
| 431740 | N75450 | Hs. 183412 | ESTs, Moderately similar to AF116721 67 | 4.00E−04 | 1.73 |
| 419652 | AL157485 | Hs. 91973 | hypothetical protein | 1.88E−04 | 1.73 |
| 453173 | AB007902 | Hs. 32168 | KIAA0442 protein | 6.67E−07 | 1.72 |
| 407690 | R47799 | Hs. 266957 | hypothetical protein FLJ14281 | 4.68E−08 | 1.72 |
| 426817 | AL122088 | Hs172627 | Homo sapiens mRNA; cDNA DKFZp564C0671 (f | 4.70E−08 | 1.72 |
| 418945 | BE246762 | Hs. 89499 | arachidonate 5-lipoxygenase | 2.44E−04 | 1.72 |
| 403828 | | | C4000447*: gi\|7705570\|ref\|NP_038851.1\| KI | 1.13E−04 | 1.72 |
| 427920 | Z11502 | Hs. 181107 | annexin A13 | 7.84E−04 | 1.72 |
| 421394 | S49953 | Hs. 103989 | DNA-binding transcriptional activator | 1.79E−03 | 1.72 |
| 447513 | AW955776 | Hs. 313500 | ESTs, Moderately similar to ALU7_HUMAN A | 3.04E−08 | 1.71 |
| 405689 | | | NM_018850*: Homo sapiens ATP-binding cass | 2.61E−04 | 1.71 |
| 441879 | AI521936 | Hs. 107149 | novel protein similar to archaeal, yeast | 4.46E−06 | 1.71 |
| 414781 | D50917 | Hs. 77293 | KIAA0127 gene product | 3.90E−05 | 1.71 |
| 444619 | BE538082 | Hs. 8172 | ESTs, Moderately similar to A46010 X-lin | 4.33E−04 | 1.71 |
| 451334 | AI122691 | Hs. 13268 | ESTs | 2.68E−05 | 1.71 |
| 446436 | BE392690 | | activating transcription factor 5 | 4.25E−08 | 1.71 |
| 423332 | AI091466 | Hs. 127241 | sorting nexin 7 | 7.16E−08 | 1.71 |
| 431660 | T30267 | Hs. 267120 | hypothetical protein DKFZp434O1427 | 1.48E−04 | 1.70 |
| 425274 | BE281191 | Hs. 155462 | minichromosome maintenance deficient (mi | 4.90E−09 | 1.70 |
| 442961 | BE614474 | | F-box only protein 22 | 4.42E−08 | 1.70 |
| 425995 | M92432 | Hs. 1974 | guanylate cyclase 2D, membrane (retina-s | 1.35E−03 | 1.70 |
| 429937 | AL080155 | Hs. 226372 | DKFZP434J154 protein | 1.69E−06 | 1.70 |
| 410660 | AI061118 | Hs. 65328 | Fanconi anemia, complementation group F | 3.51E−04 | 1.70 |
| 444866 | BE620779 | Hs. 12094 | mitochondrial ribosomal protein L30 | 8.52E−05 | 1.70 |
| 416602 | NM_006159 | Hs. 79389 | Protein kinase C-binding protein NELL2 | 5.82E−05 | 1.70 |
| 431263 | AW129203 | Hs. 322915 | ESTs | 2.31E−03 | 1.69 |
| 423201 | NM_000163 | Hs. 125180 | growth hormone receptor | 1.38E−05 | 1.69 |
| 430844 | T94960 | | gb: ye38d07.r1 Stratagene lung (937210) H | 8.59E−05 | 1.69 |
| 403742 | | | Target Exon | 9.17E−06 | 1.69 |
| 433001 | AF217513 | Hs. 279905 | clone HQ0310 PRO0310p1 | 1.05E−05 | 1.69 |
| 426214 | H59846 | Hs. 321127 | ESTs, Moderately similar to ALU7_HUMAN A | 7.93E−03 | 1.69 |
| 430007 | NM_014892 | Hs. 227602 | KIAA1116 protein | 9.67E−06 | 1.69 |
| 429689 | R77698 | Hs. 337778 | ESTs | 4.90E−04 | 1.69 |
| 424626 | AA344308 | Hs. 128427 | Homo sapiens BAC clone RP11-335J18 from | 1.61E−04 | 1.69 |
| 410678 | BE540516 | Hs. 293732 | hypothetical protein MGC3195 | 1.88E−05 | 1.69 |
| 439645 | BE091801 | Hs. 27167 | ESTs, Weakly similar to I38022 hypotheti | 7.02E−09 | 1.69 |
| 431530 | X61615 | Hs. 2798 | leukemia inhibitory factor receptor | 7.86E−05 | 1.69 |
| 434995 | AW974995 | | gb: EST387100 MAGE resequences, MAGN Homo | 2.70E−04 | 1.69 |
| 423420 | AI571364 | Hs. 128382 | Homo sapiens mRNA; cDNA DKFZp761l1224 (f | 3.52E−05 | 1.69 |
| 453210 | AL133161 | Hs. 32360 | hypothetical protein FLJ10867 | 1.57E−05 | 1.69 |
| 446096 | AI276454 | | gb: ql71a12.x1 Soares_NhHMPu_S1 Homosapi | 3.79E−04 | 1.69 |
| 433546 | AI075877 | Hs. 125461 | hypothetical protein FLJ11539 | 1.21E−03 | 1.68 |
| 435496 | AW840171 | Hs. 265398 | PAR-6 beta | 6.80E−05 | 1.68 |
| 447211 | AL161961 | Hs. 17767 | KIAA1554 protein | 1.96E−11 | 1.68 |
| 438277 | AL022326 | Hs. 6139 | synaptogyrin 1 | 4.42E−03 | 1.68 |
| 403319 | | | fms-related tyrosine kinase 3 | 4.52E−05 | 1.68 |
| 436332 | AL049679 | Hs. 82302 | Homo sapiens cDNA FLJ14814 fis, clone NT | 2.08E−05 | 1.68 |
| 434669 | AF151534 | Hs. 92023 | core histone macroH2A2.2 | 3.36E−07 | 1.68 |
| 428123 | AI702275 | Hs. 104842 | hypothetical protein FLJ23407 | 6.64E−05 | 1.68 |
| 436957 | AA902488 | Hs. 122952 | ESTs | 1.38E−05 | 1.68 |
| 421777 | BE562088 | Hs. 108196 | HSPC037 protein | 6.55E−04 | 1.68 |
| 437708 | AB033020 | Hs. 5801 | KIAA1194 protein | 8.24E−07 | 1.68 |
| 444821 | AA053564 | Hs. 12040 | STE20-like kinase | 1.25E−05 | 1.68 |
| 400170 | | | Eos Control | 3.70E−04 | 1.68 |
| 450221 | AA328102 | Hs. 24641 | cytoskeleton associated protein 2 | 9.27E−06 | 1.68 |
| 448440 | AA173467 | Hs. 62402 | p21/Cdc42/Rac1-activated kinase 1 (yeast | 1.29E−06 | 1.68 |
| 424354 | NM_014314 | Hs. 145612 | RNA helicase | 2.12E−03 | 1.68 |
| 407369 | AA989060 | Hs. 193989 | TAR DNA binding protein | 4.61E−07 | 1.68 |
| 423054 | AW449073 | Hs. 278346 | CDC2-related protein kinase 7 | 6.94E−08 | 1.67 |
| 443542 | AI927065 | Hs. 146040 | ESTs | 3.92E−05 | 1.67 |
| 452696 | AI826645 | Hs. 211534 | ESTs | 8.04E−06 | 1.67 |
| 438378 | AW970529 | Hs. 86434 | hypothetical protein FLJ21816 | 2.17E−07 | 1.67 |
| 446552 | AW470827 | Hs. 156241 | ESTs | 2.20E−06 | 1.67 |
| 417707 | AL035786 | Hs. 82425 | actin related protein 2/3 complex, subun | 1.18E−07 | 1.67 |
| 442993 | BE018682 | Hs. 166196 | ATPase, Class I, type 88, member 1 | 2.23E−07 | 1.67 |
| 430048 | T65054 | Hs. 73605 | ESTs | 3.31E−03 | 1.67 |
| 451050 | AW937420 | | ESTs | 5.05E−04 | 1.67 |
| 404602 | | | C9001493*: gi\|12735190\|ref\|XP_005622.2\| t | 1.97E−05 | 1.66 |
| 423882 | BE387203 | Hs. 134406 | hypothetical protein FLJ20511 | 2.36E−03 | 1.66 |
| 421508 | NM_004833 | Hs. 105115 | absent in melanoma 2 | 6.80E−05 | 1.66 |
| 419131 | AA406293 | Hs. 109526 | ESTs | 8.36E−06 | 1.66 |
| 452207 | NM_014517 | Hs. 28423 | upstream binding protein 1 (LBP-1a) | 1.14E−06 | 1.66 |
| 445411 | AL137255 | Hs. 12646 | hypothetical protein FLJ22693 | 2.80E−08 | 1.66 |
| 403654 | | | NM_003071: Homo sapiens SWI/SNF related, | 1.90E−04 | 1.66 |
| 423450 | AJ290445 | Hs. 128759 | KIAA0524 protein | 1.00E−03 | 1.66 |
| 430315 | NM_004293 | Hs. 239147 | guanine deaminase | 2.69E−05 | 1.66 |
| 443547 | AW271273 | | hypothetical protein FLJ12666 | 2.60E−10 | 1.66 |
| 417758 | U27699 | Hs. 82535 | solute carrier family 6 (neurotransmitte | 8.60E−07 | 1.66 |
| 417689 | AA828347 | Hs. 90998 | KIAA0128 protein; septin 2 | 3.65E−09 | 1.66 |
| 449901 | AI674072 | | gb: wd15h01.x1 Soares_NFL_T_GBC_S1 Homos | 8.34E−07 | 1.66 |
| 425905 | AB032959 | Hs. 318584 | novel C3HC4 type Zinc finger (ring finge | 1.11E−03 | 1.66 |
| 407976 | AI633875 | Hs. 77823 | hypothetical protein FLJ21343 | 2.07E−05 | 1.65 |
| 416276 | U41060 | Hs. 79136 | LIV-1 protein, estrogen regulated | 1.29E−05 | 1.65 |
| 418419 | X55039 | Hs. 85004 | centromere protein B (80 kD) | 1.89E−09 | 1.65 |
| 438384 | W22358 | Hs. 109778 | KIAA1449 protein | 9.10E−08 | 1.65 |
| 443065 | H29584 | Hs. 288198 | hypothetical protein FLJ13949 | 3.07E−06 | 1.65 |
| 438527 | AI969251 | Hs. 115325 | RAB7, member RAS oncogene family-like 1 | 2.96E−04 | 1.65 |
| 427507 | AF240467 | Hs. 179152 | toll-like receptor 7 | 5.62E−03 | 1.65 |
| 439980 | AL039706 | Hs. 19414 | ESTs, Weakly similar to S25150 gene p27 | 7.11E−05 | 1.64 |
| 419493 | AF001212 | Hs. 90744 | proteasome (prosome, macropain) 26S subu | 1.48E−04 | 1.64 |
| 437325 | AF142481 | Hs. 5548 | f-box and leucine-rich repeat protein 5 | 9.02E−08 | 1.64 |
| 435525 | AI831297 | Hs. 123310 | ESTs | 2.20E−03 | 1.64 |
| 430515 | AA746503 | Hs. 283313 | ESTs | 6.68E−05 | 1.64 |
| 410165 | BE560228 | Hs. 71869 | apoptosis-associated speck-like protein | 1.09E−06 | 1.64 |
| 425996 | W67330 | | hypothetical protein AL110115 | 6.40E−08 | 1.64 |
| 444665 | BE613126 | Hs. 47783 | B aggressive lymphoma gene | 7.26E−08 | 1.64 |
| 424960 | BE245380 | Hs. 153952 | 5′ nucleotidase (CD73) | 2.14E−04 | 1.64 |
| 449722 | BE280074 | Hs. 23960 | cyclin B1 | 2.23E−05 | 1.64 |
| 435427 | AA682573 | Hs. 188982 | ESTs, Weakly similar to organic anion tr | 4.66E−04 | 1.64 |
| 437186 | AA338305 | Hs. 5472 | hypothetical protein FLJ20173 | 1.90E−04 | 1.64 |
| 448797 | BE614460 | | ribosomal protein L17 | 6.90E−06 | 1.64 |
| 446684 | AI910777 | Hs. 125087 | ESTs | 2.60E−03 | 1.63 |
| 428413 | AI984187 | Hs. 245798 | ESTs | 4.04E−04 | 1.63 |
| 435787 | AW162767 | Hs. 100914 | hypothetical protein FLJ10352 | 3.26E−05 | 1.63 |
| 448377 | AI494514 | Hs. 21264 | ESTs | 5.24E−04 | 1.63 |
| 451684 | AF216751 | Hs. 26813 | CDA14 | 1.14E−03 | 1.63 |
| 402396 | | | Target Exon | 2.00E−04 | 1.63 |
| 441969 | AI733386 | | ESTs, Weakly similar to ALU1_HUMAN ALU S | 1.84E−04 | 1.63 |
| 421138 | AA687420 | Hs. 190145 | ESTs | 1.54E−05 | 1.63 |
| 417353 | AA375752 | Hs. 348140 | Homo sapiens mRNA; cDNA DKFZp586F1822 (f | 5.38E−04 | 1.63 |
| 410261 | AF145713 | Hs. 61490 | schwannomin-interacting protein 1 | 3.36E−04 | 1.63 |
| 458476 | AA336878 | Hs. 9842 | Human DNA sequence from clone RP4-788L20 | 4.80E−05 | 1.63 |
| 405372 | | | NM_006841: Homo sapiens transporter prote | 2.82E−04 | 1.63 |
| 452059 | AI222069 | Hs. 13015 | hypothetical protein similar to mouse Dn | 1.82E−05 | 1.63 |
| 410568 | AW162948 | Hs. 64542 | cleavage and polyadenylation specific fa | 1.28E−04 | 1.63 |
| 407502 | U52096 | | gb: Human zinc finger protein (kr-znf1) m | 2.30E−04 | 1.63 |
| 420560 | AW207748 | Hs. 59115 | ESTs | 4.40E−05 | 1.63 |
| 412530 | AA766268 | Hs. 266273 | hypothetical protein FLJ13346 | 2.49E−06 | 1.63 |
| 448888 | AW196663 | Hs. 200242 | caspase recruitment domain protein 6 | 2.43E−05 | 1.62 |
| 428220 | BE183533 | Hs. 347128 | Human DNA sequence from clone 34B21 on c | 1.64E−07 | 1.62 |
| 450621 | AW297288 | Hs. 55918 | hypothetical protein FLJ11354 | 2.40E−06 | 1.62 |
| 419437 | U61262 | Hs. 90408 | neogenin (chicken) homolog 1 | 7.24E−05 | 1.62 |
| 403694 | | | Target Exon | 8.29E−06 | 1.62 |
| 441253 | AI632744 | Hs. 129501 | ESTs | 4.42E−03 | 1.62 |
| 457770 | BE065030 | Hs. 124179 | ESTs | 7.93E−04 | 1.62 |
| 416647 | BE297139 | Hs. 79411 | replication protein A2 (32 kD) | 1.54E−06 | 1.62 |
| 410408 | NM_012260 | Hs. 63290 | 2-hydroxyphytanoyl-CoA lyase | 3.56E−05 | 1.62 |
| 430248 | AW958965 | Hs. 236774 | high-mobility group (nonhistone chromoso | 1.57E−09 | 1.62 |
| 447547 | NM_007229 | Hs. 18842 | protein kinase C and casein kinase subst | 3.40E−06 | 1.61 |
| 431863 | AA188185 | Hs. 289043 | spindlin | 1.87E−08 | 1.61 |
| 430422 | AI078115 | Hs. 54680 | ESTs | 1.05E−06 | 1.61 |
| 445657 | AW612141 | Hs. 279575 | Homo sapiens G-protein coupled receptor | 1.89E−05 | 1.61 |
| 456740 | AF068302 | Hs. 125031 | choline/ethanolaminephosphotransferase | 2.48E−05 | 1.61 |
| 410382 | AW664971 | Hs. 259546 | ESTs | 5.40E−05 | 1.61 |
| 421410 | AI472323 | Hs. 134353 | ESTs | 7.65E−05 | 1.61 |
| 438176 | AW138970 | Hs. 144759 | ESTs | 7.85E−05 | 1.61 |
| 404584 | | | Target Exon | 3.42E−03 | 1.61 |
| 422423 | AF283777 | Hs. 116481 | CD72 antigen | 1.72E−03 | 1.61 |
| 441894 | AA134329 | | Homo sapiens, clone IMAGE: 3685398, mRNA, | 7.36E−04 | 1.61 |
| 421801 | AI431597 | Hs. 284294 | Breakpoint cluster region protein, uteri | 2.69E−05 | 1.61 |
| 428379 | X06026 | Hs. 2259 | CD3G antigen, gamma polypeptide (TiT3 co | 8.68E−05 | 1.61 |
| 451788 | BE242857 | Hs. 27021 | hypothetical protein FLJ11159 | 2.35E−06 | 1.61 |
| 444708 | AW971049 | Hs. 11774 | protein (peptidyl-prolyl cis/trans isome | 9.22E−05 | 1.61 |
| 442358 | BE567985 | Hs. 18585 | ESTs, Moderately similar to ALU4_HUMAN A | 3.12E−06 | 1.60 |
| 422521 | AW363324 | Hs. 126906 | hypothetical protein FLJ 12598 | 1.19E−07 | 1.60 |
| 428475 | AF172940 | Hs. 184542 | CGI-127 protein | 1.11E−06 | 1.60 |
| 449027 | AJ271216 | Hs. 22880 | dipeptidylpeptidase III | 7.55E−04 | 1.60 |
| 404240 | | | NM_018950: Homo sapiens major histocompat | 3.66E−09 | 1.60 |
| 414792 | BE314949 | Hs. 87128 | hypothetical protein FLJ23309 | 1.12E−08 | 1.60 |
| 416747 | AW876523 | Hs. 15929 | hypothetical protein FLJ12910 | 9.45E−06 | 1.60 |
| 418001 | R80582 | Hs. 24625 | ESTs | 1.62E−03 | 1.60 |
| 433009 | AA761668 | | gb: nz24c08.s1 NCI_CGAP_GCB1 Homo sapiens | 6.37E−03 | 1.60 |
| 421016 | AA504583 | Hs. 101047 | transcription factor 3 (E2A immunoglobul | 9.01E−06 | 1.60 |
| 436256 | T87113 | Hs. 193825 | ESTs | 1.46E−04 | 1.60 |
| 428418 | AI368826 | Hs. 8768 | ESTs | 1.04E−06 | 1.60 |
| 451253 | H48299 | Hs. 26126 | claudin 10 | 1.22E−03 | 1.60 |
| 425913 | AA365799 | | SEC22, vesicle trafficking protein (S. c | 1.11E−06 | 1.60 |
| 418460 | M26315 | Hs. 85258 | CD8 antigen, alpha polypeptide (p32) | 4.30E−04 | 1.60 |
| 420680 | AA648758 | Hs. 187791 | ESTs | 2.56E−04 | 1.60 |
| 425691 | NM_005328 | Hs. 159226 | hyaluronan synthase 2 | 3.04E−03 | 1.60 |
| 446307 | T50083 | Hs. 22247 | ESTs | 5.05E−03 | 1.60 |
| 422805 | AA436989 | Hs. 121017 | H2A histone family, member A | 2.11E−05 | 1.60 |
| 420092 | AA814043 | Hs. 88045 | ESTs | 2.86E−03 | 1.59 |
| 443959 | AL120546 | | gb: DKFZp761A069_r1 761 (synonym: hamy2) | 3.44E−04 | 1.59 |
| 404409 | | | C8001172*: gi\|10432395\|emb\|CAC10285.1\| (A | 5.86E−04 | 1.59 |
| 405601 | | | Target Exon | 5.21E−04 | 1.59 |
| 431543 | AW969619 | Hs. 259768 | adenylate cyclase 1 (brain) | 1.20E−03 | 1.59 |
| 428826 | AL048842 | Hs. 194019 | atlractin | 3.04E−07 | 1.59 |
| 439498 | AA908731 | Hs. 58297 | CLLL8 protein | 2.01E−04 | 1.59 |
| 438661 | AA813539 | Hs. 123414 | ESTs, Weakly similar to I37570 zinc fing | 7.79E−06 | 1.59 |
| 447105 | AW377610 | Hs. 11123 | DKFZP564G092 protein | 2.19E−05 | 1.59 |
| 412983 | AI478828 | Hs. 16570 | ESTs, Weakly similar to I38022 hypotheti | 6.13E−05 | 1.59 |
| 440764 | AA905406 | Hs. 9905 | ESTs, Weakly similar to unnamed protein | 1.78E−03 | 1.59 |
| 452806 | AW014549 | Hs. 58373 | ESTs | 4.21E−04 | 1.59 |
| 404919 | | | ENSP00000237577*: Polycystin 2 (Autosomal | 5.59E−03 | 1.59 |
| 424737 | BE301883 | Hs. 152707 | glioblastoma amplified sequence | 7.59E−05 | 1.59 |
| 435205 | X54136 | Hs. 181125 | immunoglobulin lambda locus | 5.77E−03 | 1.59 |
| 403738 | | | C4000675*: gi\|3426332\|gb\|AAC32272.1\| (AF0 | 4.04E−09 | 1.59 |
| 408705 | AA312135 | Hs. 46967 | HSPCO34 protein | 7.26E−09 | 1.59 |
| 431377 | AW178807 | Hs. 246182 | ESTs | 2.46E−03 | 1.58 |
| 433359 | H70007 | | gb: yr89e07.r1 Soares fetal liver spleen | 2.39E−03 | 1.58 |
| 447809 | AW207605 | Hs. 164230 | ESTs, Highly similar to JC7266 3′,5′-cyc | 3.29E−08 | 1.58 |
| 415938 | BE383507 | Hs. 78921 | A kinase (PRKA) anchor protein 1 | 1.35E−06 | 1.58 |
| 434375 | BE277910 | Hs. 3833 | 3′-phosphoadenosine 5′-phosphosulfate sy | 7.89E−08 | 1.58 |
| 408412 | AW193033 | Hs. 124436 | ESTs | 9.63E−04 | 1.58 |
| 419672 | AA465113 | Hs. 23853 | ESTs, Weakly similar to A34615 profilagg | 4.60E−04 | 1.58 |
| 435931 | AI077464 | | RNA binding motif protein 9 | 1.37E−04 | 1.58 |
| 432645 | 014041 | Hs. 347340 | H-2K binding factor-2 | 3.91E−03 | 1.58 |
| 412886 | AF041163 | Hs. 74647 | Human T-cell receptor active alpha-chain | 3.77E−06 | 1.58 |
| 424696 | BE439547 | Hs. 151903 | GrpE-like protein cochaperone | 2.03E−04 | 1.57 |
| 446725 | AL050173 | Hs. 16007 | chromosome 21 open reading frame 25 | 1.32E−05 | 1.57 |
| 439788 | N71241 | Hs. 119275 | ESTs | 1.99E−03 | 1.57 |
| 431736 | AI912234 | Hs. 3297 | ribosomal protein S27a | 2.14E−05 | 1.57 |
| 449368 | AW009668 | Hs. 232284 | ESTs | 1.48E−03 | 1.57 |
| 452866 | R26969 | Hs. 268016 | Homo sapiens cDNA: FLJ21243 fis, clone C | 4.10E−05 | 1.57 |
| 429586 | T73510 | Hs. 209153 | angiopoietin-like 3 | 4.09E−07 | 1.57 |
| 453887 | BE564037 | Hs. 36237 | hypothetical protein | 4.59E−06 | 1.57 |
| 428759 | AC007059 | Hs. 193065 | hypothetical protein DKFZp547M136 simila | 8.66E−06 | 1.57 |
| 401847 | | | C17000828: gi\|1666071\|emb\|CAA69174.1\| (Y0 | 6.56E−05 | 1.57 |
| 434747 | M837085 | | ESTs | 4.24E−03 | 1.57 |
| 449601 | AA461509 | Hs. 293565 | ESTs, Weakly similar to putative p150 [H | 4.36E−05 | 1.57 |
| 457758 | AI475671 | Hs. 88607 | ESTs, Highly similar to F-box protein FB | 3.83E−04 | 1.57 |
| 447574 | AF162666 | Hs. 18895 | tousled-Iike kinase 1 | 7.59E−07 | 1.57 |
| 401928 | | | Target Exon | 2.68E−03 | 1.57 |
| 406274 | | | Target Exon | 6.32E−05 | 1.56 |
| 449291 | BE176893 | Hs. 23440 | KIAA1105 protein | 1.17E−06 | 1.56 |
| 429280 | AI445960 | Hs. 3685 | hypothetical protein FLJ20209 | 5.66E−09 | 1.56 |
| 419946 | AI298508 | Hs. 18963 | ESTs | 1.24E−04 | 1.56 |
| 439008 | AF075072 | Hs. 167535 | ESTs, Weakly similar to ALU1_HUMAN ALU S | 3.38E−03 | 1.56 |
| 444756 | AA278501 | Hs. 200332 | hypothetical protein FLJ20651 | 5.36E−03 | 1.56 |
| 425409 | H18021 | Hs. 25005 | hypothetical protein MGC3329 | 1.55E−03 | 1.56 |
| 453387 | AI990741 | Hs. 252809 | ESTs | 5.19E−04 | 1.56 |
| 438156 | Z20244 | Hs. 192906 | ESTs | 3.43E−05 | 1.56 |
| 401558 | | | ENSP00000220478*: SECRETOGRANIN III. | 1.17E−04 | 1.56 |
| 420807 | M280627 | Hs. 57846 | ESTs | 6.07E−04 | 1.56 |
| 434942 | M021562 | Hs. 347950 | Homo sapiens. Similar to nuclear localiz | 3.13E−06 | 1.55 |
| 444479 | AA194980 | Hs. 30818 | Homo sapiens cDNA FLJ13681 fis. clone PL | 2.39E−03 | 1.55 |
| 444078 | BE246919 | Hs. 10290 | U5 snRNP-specific 40 KDa protein (hPrp8- | 6.70E−04 | 1.55 |
| 421612 | AF161254 | Hs. 106196 | 8D6 antigen | 7.97E−10 | 1.55 |
| 432740 | AF061034 | Hs. 278898 | tumor necrosis factor alpha-inducible ce | 1.66E−07 | 1.55 |
| 456563 | AA989220 | Hs. 766 | ESTs | 1.09E−04 | 1.55 |
| 420573 | AA278407 | Hs. 88528 | ESTs | 4.79E−03 | 1.55 |
| 437998 | U92025 | Hs. 200680 | ESTs, Moderately similar to KIAA0573 pro | 3.78E−03 | 1.55 |
| 423570 | AW838306 | Hs. 129819 | hypothetical protein FLJ11160 | 2.25E−05 | 1.55 |
| 425356 | BE244879 | Hs. 155939 | inositol polyphosphate-5-phosphatase, 14 | 6.94E−04 | 1.55 |
| 443661 | AA336609 | Hs. 10862 | Homo sapiens cDNA: FLJ23313 fis, clone H | 1.04E−04 | 1.55 |
| 433878 | AA613245 | Hs. 259181 | ESTs | 4.25E−04 | 1.55 |
| 449337 | AW772245 | Hs. 223978 | ESTs | 2.97E−05 | 1.55 |
| 434608 | AA805443 | Hs. 179909 | hypothetical protein FLJ22995 | 3.60E−04 | 1.55 |
| 418310 | AA814100 | Hs. 86693 | ESTs | 9.87E−04 | 1.54 |
| 436388 | AI806172 | Hs. 166486 | Homo sapiens cDNA FLJ11432 fis, clone HE | 4.35E−06 | 1.54 |
| 417848 | AA206581 | Hs. 116586 | ESTs, Weakly similar to JC5314 CDC28/cdc | 1.02E−05 | 1.54 |
| 435438 | H84421 | Hs. 4890 | ubiquitin-conjugating enzyme E2E 3 (homo | 2.83E−04 | 1.54 |
| 427790 | NM_002887 | Hs. 180832 | hypothetical protein MGC8641 | 2.02E−05 | 1.54 |
| 442968 | AK000606 | Hs. 8868 | golgi SNAP receptor complex member 1 | 3.73E−09 | 1.54 |
| 416727 | W05023 | | gb: za45a11.ri Soares fetal liver spleen | 4.98E−03 | 1.54 |
| 440201 | AL359588 | Hs. 7041 | hypothetical protein DKFZp762B226 | 4.87E−04 | 1.54 |
| 415670 | AA166633 | Hs. 117183 | gb: zq39b01.r1 Stratagene hNT neuron (937 | 4.54E−04 | 1.54 |
| 408380 | AF123050 | Hs. 44532 | diubiquitin | 3.89E−09 | 1.54 |
| 403746 | | | ENSP00000226812*: KIAA1494 protein (Fragm | 9.67E−04 | 1.53 |
| 420172 | AA601122 | Hs. 95655 | secreted and transmembrane 1 | 5.53E−03 | 1.53 |
| 415200 | AL040328 | Hs. 78202 | SWI/SNF related, matrix associated, acti | 6.23E−08 | 1.53 |
| 441488 | AW450935 | Hs. 7862 | hypothetical protein FLJ20312 | 7.06E−07 | 1.53 |
| 413859 | AW992356 | Hs. 8364 | Homo sapiens pyruvate dehydrogenase kina | 1.25E−04 | 1.53 |
| 425601 | AW629485 | Hs. 140720 | GSK-3 binding protein FRAT2 | 1.63E−06 | 1.53 |
| 439704 | AW020018 | Hs. 293267 | ESTs | 2.65E−05 | 1.53 |
| 409344 | R47279 | Hs. 285673 | hypothetical protein FLJ20950 | 2.38E−06 | 1.53 |
| 424602 | AK002055 | Hs. 151046 | hypothetical protein FLJ11193 | 1.23E−04 | 1.53 |
| 429556 | AW139399 | Hs. 98988 | ESTs | 2.67E−03 | 1.53 |
| 446756 | AW028485 | Hs. 26136 | hypothetical protein MGC14156 | 5.35E−07 | 1.53 |
| 418827 | BE327311 | Hs. 47166 | HT021 | 1.00E−05 | 1.53 |
| 438204 | AI589645 | Hs. 128690 | ESTs | 2.96E−04 | 1.53 |
| 417377 | NM_016603 | Hs. 82035 | potential nuclear protein C5ORF5; GAP-li | 3.15E−08 | 1.52 |
| 422536 | AA311915 | Hs. 187726 | gb: EST182621 Jurkat T-cells VI Homo sapi | 6.56E−05 | 1.52 |
| 431945 | AW000827 | Hs. 11962 | apoptosis regulator BCL-G | 3.53E−03 | 1.52 |
| 421025 | AW958975 | Hs. 29397 | Homo sapiens cDNA FLJ13226 fis. clone OV | 7.11E−06 | 1.52 |
| 444652 | BE513613 | Hs. 11538 | actin related protein 2/3 complex, subun | 7.17E−03 | 1.52 |
| 458345 | AI864078 | Hs. 144076 | ESTs | 3.45E−04 | 1.52 |
| 425692 | D90041 | Hs. 155956 | N-acetyltransferase 1 (arylamine N-acety | 2.04E−06 | 1.52 |
| 400120 | | | Eos Control | 8.78E−04 | 1.52 |
| 440591 | AA431599 | Hs. 132799 | hypothetical protein FLJ23451 | 7.90E−05 | 1.52 |
| 412826 | BE001722 | Hs. 14877 | ESTs, Weakly similar to (defline not ava | 1.45E−03 | 1.52 |
| 408683 | R58665 | Hs. 46847 | TRAF and TNF receptor-associated protein | 2.29E−09 | 1.52 |
| 424852 | AI222779 | Hs. 144848 | ESTs | 9.90E−03 | 1.52 |
| 423694 | NM_003747 | Hs. 131814 | tankyrase, TRF1-interacting ankyrin-rela | 2.70E−08 | 1.52 |
| 432134 | AI816782 | Hs. 122583 | hypothetical protein FLJ21934 | 7.31E−06 | 1.52 |
| 447131 | NM_004585 | Hs. 17466 | retinoic acid receptor responder (tazaro | 3.80E−06 | 1.52 |
| 427212 | AW293849 | Hs. 58279 | ESTs, Weakly similar to ALU7_HUMAN ALU S | 4.87E−06 | 1.52 |
| 423524 | AF055989 | Hs. 129738 | potassium voltage gated channel, Shaw-re | 4.04E−04 | 1.52 |
| 457933 | AA759062 | Hs. 121434 | ESTs | 1.63E−03 | 1.52 |
| 425769 | U72513 | Hs. 159486 | Human RPL13-2 pseudogene mRNA, complete | 3.40E−06 | 1.51 |
| 431262 | NM_006672 | Hs. 251395 | solute carrier family 22 (organic anion | 6.08E−05 | 1.51 |
| 457764 | AW028284 | Hs. 4815 | nudix (nucleoside diphosphate linked moi | 3.11E−04 | 1.51 |
| 438712 | AW978161 | Hs. 268555 | 5′-3′ exoribonuclease 2 | 1.00E−03 | 1.51 |
| 420514 | N32301 | Hs. 102496 | ESTs | 6.01E−04 | 1.51 |
| 410972 | AW812259 | | gb: RC0-ST0174-191099-031-b05 ST0174 Homo | 2.97E−03 | 1.51 |
| 446493 | AK001389 | Hs. 15144 | hypothetical protein DKFZp564O043 | 2.66E−06 | 1.51 |
| 415000 | AW025529 | Hs. 239812 | Homo sapiens serologically defined breas | 8.80E−08 | 1.51 |
| 427647 | W19744 | Hs. 180059 | Homo sapiens cDNA FLJ20653 fis, clone KA | 7.28E−06 | 1.50 |
| 409022 | BE222648 | Hs. 241432 | ESTs, Highly similar to C380A1.1b [H. sap | 3.80E−06 | 1.50 |
| 441980 | AK001441 | Hs. 8055 | hypothetical protein FLJ10579 | 1.53E−03 | 1.50 |
| 445424 | AB028945 | Hs. 12696 | cortactin SH3 domain-binding protein | 9.78E−07 | 1.50 |
| 414948 | C15240 | Hs. 182155 | ESTs | 2.68E−06 | 1.50 |
| 442878 | AI868648 | Hs. 22315 | ESTs | 7.00E−05 | 1.50 |
| 438021 | AV653790 | | WW domain-containing protein 1 | 1.28E−06 | 1.50 |
| 426723 | AW003069 | Hs. 183860 | ESTs | 7.02E−06 | 1.50 |
| 420847 | AI356920 | Hs. 23510 | ESTs | 2.14E−03 | 1.50 |
| 406972 | M32053 | | gb: Human H19 RNA gene, complete cds. | 6.00E−03 | 1.50 |
| 445309 | AL157474 | Hs. 12504 | likely ortholog of mouse Arkadia | 3.14E−05 | 1.50 |
| 421712 | AK000140 | Hs. 107139 | hypothetical protein | 9.42E−09 | 1.50 |
| 414091 | T83742 | Hs. 334616 | gb: yd67g02.s1 Soares fetal liver spleen | 2.35E−05 | 1.50 |
| 402945 | | | Target Exon | 1.02E−03 | 1.50 |
| 400487 | | | ENSP00000238977*: lnterferon-induced prot | 1.77E−04 | 1.50 |
| 438308 | AI343469 | Hs. 127685 | KIAA1627 protein | 7.96E−06 | 1.50 |
| 411227 | AW833258 | | gb: RC2-TT0007-131099-011-b10_1 TT0007 Ho | 7.01E−04 | 1.50 |
| 451452 | BE560065 | Hs. 26433 | dolichyl-phosphate(UDP-N-acetylglucosam | 1.34E−04 | 1.49 |
| 451806 | NM_003729 | Hs. 27076 | RNA 3′-terminal phosphate cyclase | 1.25E−04 | 1.49 |
| 424924 | AL039103 | Hs. 153834 | pumilio (Drosophila) homolog 1 | 3.26E−06 | 1.49 |
| 452136 | AU076713 | Hs. 479 | RAB5C, member RAS oncogene family | 4.25E−03 | 1.49 |
| 439375 | AA689526 | Hs. 344249 | steroid dehydrogenase homolog | 3.64E−06 | 1.49 |
| 410177 | NM_015425 | Hs. 59475 | DKFZP586M0122 protein | 1.00E−03 | 1.49 |
| 407813 | AL120247 | Hs. 40109 | KIAA0872 protein | 1.86E−05 | 1.49 |
| 420505 | AW967984 | Hs. 122552 | G-2 and S-phase expressed 1 | 4.16E−03 | 1.49 |
| 456373 | BE247706 | Hs. 89751 | membranE−spanning 4-domains, subfamily A | 4.10E−03 | 1.49 |
| 412853 | M34175 | Hs. 74626 | adaptor-related protein complex 2, beta | 2.89E−04 | 1.49 |
| 434369 | AI650363 | Hs. 116462 | ESTs | 1.34E−03 | 1.49 |
| 442751 | AI358404 | | gb: qw20f03.x1 NCI_CGAP_Ut3 Homo sapiens | 2.61E−05 | 1.49 |
| 442432 | BE093589 | Hs. 38178 | hypothetical protein FLJ23468 | 3.18E−06 | 1.49 |
| 408784 | AW971350 | Hs. 63386 | ESTs | 9.38E−05 | 1.49 |
| 449629 | AA031547 | Hs. 298442 | adaptor-related protein complex 3, mu 1 | 4.62E−05 | 1.49 |
| 419110 | AA234171 | Hs. 187626 | ESTs | 2.44E−04 | 1.49 |
| 457238 | U07358 | Hs. 211601 | mitogen-activated protein kinase kinase | 2.21E−03 | 1.49 |
| 408483 | AA464836 | Hs. 291079 | ESTs, Weakly similar to T27173 hypotheti | 5.75E−07 | 1.49 |
| 456633 | AW139036 | Hs. 108957 | 40S ribosomal protein S27 isoform | 6.06E−04 | 1.48 |
| 450746 | D82673 | Hs. 278589 | general transcription factor II, i | 1.58E−09 | 1.48 |
| 438637 | BE500941 | Hs. 126730 | ESTs, Weakly similar to KIAA1214 protein | 1.08E−03 | 1.48 |
| 444775 | AI040384 | Hs. 19102 | ESTs, Weakly similar to organic anion tr | 1.50E−07 | 1.48 |
| 426335 | AI054347 | Hs. 2017 | ribosomal protein L38 | 6.98E−03 | 1.48 |
| 450253 | AL133047 | Hs. 24715 | Homo sapiens mRNA; cDNA DKFZp434D0215 (f | 4.78E−05 | 1.48 |
| 434466 | AB037829 | Hs. 3862 | regulator of nonsense transcripts 2; DKF | 2.91E−06 | 1.48 |
| 446164 | AW273539 | | hypothetical protein FLJ23577 | 3.00E−03 | 1.48 |
| 458533 | AA314487 | Hs. 11123 | DKFZP564G092 protein | 7.99E−05 | 1.48 |
| 450377 | AB033091 | | KIAA1265 protein | 7.22E−05 | 1.48 |
| 411798 | AW864049 | Hs. 133505 | gb: PM2-SN0013-120400-003-c06 SN0013 Homo | 1.10E−03 | 1.48 |
| 419715 | AF070523 | Hs. 92384 | vitamin A responsive; cytoskeleton relat | 4.88E−07 | 1.48 |
| 422611 | AA158177 | Hs. 118722 | fucosyltransferase 8 (alpha (1, 6) fucosy | 1.93E−06 | 1.48 |
| 453186 | AK001708 | Hs. 32271 | hypothetical protein FLJ10846 | 3.21E−04 | 1.48 |
| 403330 | | | Target Exon | 4.62E−05 | 1.47 |
| 447095 | AI361424 | Hs. 265174 | ESTs | 1.74E−03 | 1.47 |
| 445175 | AV652851 | Hs. 20255 | ESTs | 3.04E−05 | 1.47 |
| 406868 | AA505445 | Hs. 300697 | immunoglobulin heavy constant gamma 3 (G | 1.24E−04 | 1.47 |
| 424683 | N87519 | Hs. 27196 | ESTs | 1.58E−05 | 1.47 |
| 453394 | AW960474 | Hs. 40289 | ESTs | 5.61E−06 | 1.47 |
| 416744 | AL135579 | Hs. 79709 | phosphotidylinositol transfer protein | 3.24E−07 | 1.47 |
| 416206 | AW206248 | Hs. 111092 | hypothetical protein FLJ22332 | 1.53E−09 | 1.47 |
| 400975 | | | Target Exon | 1.06E−03 | 1.47 |
| 452068 | W76412 | Hs. 57877 | ESTs | 1.45E−04 | 1.47 |
| 444985 | AI677737 | Hs. 28329 | hypothetical protein FLJ14005 | 3.04E−05 | 1.47 |
| 430945 | U80669 | Hs. 55999 | NK homeobox (Drosophila), family 3. A | 1.72E−05 | 1.47 |
| 424180 | AB032987 | Hs. 320861 | KIAA1161 protein | 1.08E−04 | 1.47 |
| 446189 | H85224 | Hs. 214013 | ESTs | 4.67E−05 | 1.47 |
| 433991 | AA912749 | | ESTs | 7.45E−03 | 1.47 |
| 412972 | AA771898 | Hs. 33412 | ESTs | 2.53E−03 | 1.47 |
| 433418 | AA587831 | Hs. 87134 | ESTs | 2.47E−03 | 1.47 |
| 429520 | AA160142 | Hs. 205058 | hypothetical protein FLJ20075 | 2.48E−03 | 1.46 |
| 452852 | AK001972 | Hs. 30822 | hypothetical protein FLJ11110 | 1.77E−08 | 1.46 |
| 419083 | AI479560 | Hs. 98613 | Homo sapiens cDNA FLJ12292 fis. clone MA | 5.14E−06 | 1.46 |
| 422643 | D42045 | Hs. 1560 | DNA-crosslink repair gene SNM1 | 2.05E−04 | 1.46 |
| 421965 | AA301100 | Hs. 346482 | gb: EST14128 Testis tumor Homo sapiens cD | 1.07E−06 | 1.46 |
| 448277 | BE622827 | Hs. 99486 | hypothetical protein FLJ13044 | 1.67E−07 | 1.46 |
| 423979 | AF229181 | Hs. 136644 | CS box-containing WD protein | 1.77E−03 | 1.46 |
| 459169 | AI905517 | | gb: RC-BT091-210199-105 BT091 Homo sapien | 1.19E−03 | 1.46 |
| 442220 | AL037800 | Hs. 8148 | selenoprotein T | 1.41E−05 | 1.46 |
| 407807 | AL031427 | Hs. 40094 | Human DNA sequence from clone 167A19 on | 5.25E−04 | 1.46 |
| 426642 | AW068223 | Hs. 171581 | ubiquitin C-terminal hydrolase UCH37 | 4.71E−06 | 1.46 |
| 442348 | BE315496 | Hs. 8258 | DKFZP434D1335 protein | 1.18E−03 | 1.46 |
| 443487 | AI073491 | Hs. 269887 | ESTs, Highly similar to KPBB_HUMAN PHOSP | 1.03E−03 | 1.46 |
| 419586 | AI088485 | Hs. 144759 | ESTs, Weakly similar to I38022 hypotheti | 6.52E−08 | 1.46 |
| 433514 | AW183734 | Hs. 189782 | hypothetical protein FLJ10747 | 7.13E−04 | 1.46 |
| 417321 | N68722 | Hs. 191368 | ESTs | 6.13E−04 | 1.46 |
| 408521 | AA055264 | Hs. 260848 | ESTs, Weakly similar to S23650 retroviru | 7.91E−07 | 1.45 |
| 438510 | AL080220 | Hs. 6285 | DKFZP586P0123 protein | 1.59E−05 | 1.45 |
| 441458 | AW815108 | | gb: QV4-ST0212-091199-023-h01 ST0212 Homo | 4.05E−04 | 1.45 |
| 420568 | F09247 | Hs. 247735 | protocadherin alpha 10 | 1.65E−05 | 1.45 |
| 431186 | NM_012249 | Hs. 250697 | ras-like protein | 4.34E−07 | 1.45 |
| 451987 | AA815092 | Hs. 77554 | Homo sapiens cDNA FLJ14967 fis, clone TH | 6.89E−04 | 1.45 |
| 419998 | AA252691 | Hs. 346200 | gb: zs26d09.r1 NCI_CGAP_GCB1 Homo sapiens | 2.32E−03 | 1.45 |
| 421272 | AA704157 | | ESTs | 2.59E−04 | 1.45 |
| 400200 | | | NM_002788*: Homo sapiens proteasome (pros | 7.15E−05 | 1.45 |
| 400440 | X83957 | Hs. 83870 | nebulin | 3.35E−03 | 1.45 |
| 444654 | AV650572 | Hs. 23440 | KIAA1105 protein | 6.77E−06 | 1.45 |
| 446793 | AW590131 | Hs. 156468 | ESTs | 5.34E−03 | 1.45 |
| 445919 | T53519 | Hs. 334692 | hypothetical protein MGC14141 | 5.08E−03 | 1.45 |
| 433422 | AW976627 | Hs. 284294 | Breakpoint cluster region protein, uteri | 2.98E−07 | 1.45 |
| 421721 | AB037799 | Hs. 107279 | KIAA1378 protein | 4.59E−03 | 1.45 |
| 444913 | AI362726 | Hs. 193656 | Homo sapiens mRNA for KIAA1658 protein, | 1.15E−06 | 1.45 |
| 421957 | AW068637 | Hs. 109857 | hypothetical protein DKFZp434H0820 | 8.29E−03 | 1.45 |
| 412935 | BE267045 | Hs. 75064 | tubulin-specific chaperone c | 4.27E−08 | 1.44 |
| 413884 | AI668892 | Hs. 239758 | hypothetical protein FLJ12389 similar to | 1.04E−04 | 1.44 |
| 417961 | Z44190 | Hs. 83023 | peroxisomal biogenesis factor 11B | 1.35E−07 | 1.44 |
| 442619 | AA447492 | Hs. 20183 | ESTs, Weakly similar to AF164793 1 prote | 3.67E−06 | 1.44 |
| 439894 | AA853077 | Hs. 300697 | immunoglobulin heavy constant gamma 3 (G | 7.44E−03 | 1.44 |
| 422241 | Y00062 | Hs. 170121 | protein tyrosine phosphatase, receptor t | 1.57E−08 | 1.44 |
| 407687 | AK002011 | Hs. 37558 | hypothetical protein FLJ11149 | 8.19E−04 | 1.44 |
| 427929 | BE613835 | Hs. 181159 | Homo sapiens mRNA; cDNA DKFZp434F0217 (f | 2.28E−05 | 1.44 |
| 410054 | AL120050 | Hs. 58220 | Homo sapiens cDNA: FLJ23005 fis, clone L | 1.21E−03 | 1.44 |
| 443639 | BE269042 | Hs. 9661 | proteasome (prosome, macropain) subunit, | 4.08E−07 | 1.44 |
| 449437 | AI702038 | Hs. 100057 | Homo sapiens cDNA: FLJ22902 fis, clone K | 2.51E−05 | 1.44 |
| 406918 | M88357 | | gb: Homo sapiens DNA-binding protein (ZNF | 6.10E−03 | 1.44 |
| 424286 | AA338285 | Hs. 90744 | proteasome (prosome, macropain) 26S subu | 2.50E−07 | 1.44 |
| 425261 | BE385099 | | hypothetical protein MGC3017 | 1.12E−04 | 1.44 |
| 452822 | X85689 | Hs. 288617 | hypothetical protein FLJ22621 | 6.51E−04 | 1.44 |
| 425289 | AW139342 | Hs. 155530 | interferon, gamma-inducible protein 16 | 8.16E−06 | 1.44 |
| 428894 | AA437066 | Hs. 271736 | ESTs | 2.54E−03 | 1.44 |
| 418533 | NM_004533 | Hs. 85937 | myosin-binding protein C, fast-type | 8.68E−03 | 1.43 |
| 411030 | BE387193 | Hs. 67896 | 7-60 protein | 8.65E−06 | 1.43 |
| 435882 | AW194987 | | ESTs | 2.85E−04 | 1.43 |
| 444773 | BE156256 | Hs. 11923 | hypothetical protein | 6.80E−03 | 1.43 |
| 435522 | N64214 | Hs. 9774 | synovial sarcoma translocation gene on c | 2.42E−07 | 1.43 |
| 400552 | | | Target Exon | 2.09E−04 | 1.43 |
| 449103 | T24968 | Hs. 23038 | HSPC071 protein | 6.24E−06 | 1.43 |
| 411492 | T46848 | Hs. 70337 | immunoglobulin superfamily, member 4 | 2.14E−06 | 1.43 |
| 450456 | AA600192 | | ESTs, Weakly similar to ALUB_HUMAN !!!! | 6.66E−06 | 1.43 |
| 440515 | AJ131245 | Hs. 7239 | SEC24 (S. cerevisiae) related gene famil | 3.40E−03 | 1.43 |
| 434924 | AA443164 | Hs. 23259 | hypothetical protein FLJ13433 | 7.81E−07 | 1.43 |
| 439981 | AI348408 | Hs. 124675 | ESTs, Weakly similar to T14742 hypotheti | 3.16E−07 | 1.43 |
| 452268 | NM_003512 | HS. 28777 | H2A histone family, member L | 6.16E−09 | 1.43 |
| 408358 | D20044 | Hs. 12929 | hypothetical protein FLJ20721 | 2.62E−04 | 1.42 |
| 446755 | AW451473 | Hs. 16134 | serine/threonine kinase 10 | 1.34E−03 | 1.42 |
| 435772 | AA700019 | Hs. 132992 | ATP-binding cassette, sub-family G (WHIT | 1.62E−05 | 1.42 |
| 457250 | AA811987 | Hs. 125779 | ESTs | 5.09E−03 | 1.42 |
| 415192 | D17793 | Hs. 78183 | aldo-keto reductase family 1, member C3 | 1.35E−05 | 1.42 |
| 418506 | AA084248 | Hs. 85339 | G protein-coupled receptor 39 | 1.28E−03 | 1.42 |
| 407972 | AA827639 | Hs. 18587 | KIAA1588 protein | 8.57E−03 | 1.42 |
| 452718 | AI762301 | Hs. 44017 | sirtuin (silent mating type information | 1.90E−04 | 1.42 |
| 443325 | BE398006 | Hs. 90462 | Homo sapiens, clone IMAGE: 4132043, mRNA, | 3.44E−03 | 1.42 |
| 422040 | AA172106 | Hs. 110950 | Rag C protein | 9.07E−05 | 1.42 |
| 442296 | NM_007275 | Hs. 8186 | lung cancer candidate | 1.13E−05 | 1.42 |
| 418475 | AI858732 | Hs. 30443 | sentrin/SUMO-specific protease | 1.94E−04 | 1.42 |
| 403147 | | | Target Exon | 4.29E−03 | 1.42 |
| 452647 | N29495 | Hs. 96870 | staufen (Drosophila, RNA-binding protein | 4.56E−03 | 1.42 |
| 415049 | N67334 | Hs. 50158 | ESTs | 1.54E−03 | 1.41 |
| 406605 | | | Target Exon | 9.18E−05 | 1.41 |
| 434152 | AI792665 | Hs. 291190 | ESTs | 1.11E−03 | 1.41 |
| 433307 | AI393009 | Hs. 153385 | hypothetical protein MGC5509 | 6.73E−04 | 1.41 |
| 414245 | BE148072 | Hs. 75850 | WAS protein family, member 1 | 1.25E−03 | 1.41 |
| 419407 | AW410377 | Hs. 41502 | hypothetical protein FLJ21276 | 2.41E−07 | 1.41 |
| 414548 | AW937036 | Hs. 183506 | hypothetical protein FLJ14213 | 5.51E−04 | 1.41 |
| 421958 | AA357185 | Hs. 109918 | ras homolog gene family, member H | 3.31E−04 | 1.41 |
| 450122 | BE313765 | | ESTs, Weakly similar to I38022 hypotheti | 6.86E−06 | 1.41 |
| 445308 | AW975748 | Hs. 5724 | sclerostin | 5.31E−05 | 1.41 |
| 434045 | AI065133 | Hs. 152316 | hypothetical protein PRO0971 | 1.76E−04 | 1.41 |
| 423262 | NM_005479 | Hs. 126057 | frequently rearranged in advanced T-cell | 1.49E−07 | 1.41 |
| 451413 | AA448974 | Hs. 26367 | PC3-96 protein | 1.90E−06 | 1.41 |
| 434978 | AA321238 | Hs. 4310 | eukaryotic translation initiation factor | 2.64E−03 | 1.41 |
| 417100 | AI828908 | Hs. 4552 | ubiquilin 2 | 1.80E−04 | 1.41 |
| 445692 | AI248322 | Hs. 182099 | ESTs | 2.29E−05 | 1.41 |
| 436486 | AA742221 | Hs. 120633 | ESTs | 3.93E−03 | 1.41 |
| 441976 | AA428403 | Hs. 106131 | ESTs | 1.08E−05 | 1.41 |
| 445530 | BE294791 | | hypothetical protein FLJ12666 | 3.72E−04 | 1.41 |
| 402907 | | | NM_024777*: Homo sapiens hypothetical pro | 9.75E−03 | 1.41 |
| 419165 | AW860767 | Hs. 118879 | ESTs | 1.90E−03 | 1.41 |
| 406815 | AA833930 | | tRNA isopentenylpyrophosphate transferas | 1.03E−03 | 1.41 |
| 421726 | AK001237 | Hs. 319088 | hypothetical protein FLJ10375 | 1.04E−07 | 1.41 |
| 437392 | AL359943 | Hs. 132272 | Homo sapiens mRNA; cDNA DKFZp762D186 (fr | 3.68E−03 | 1.41 |
| 439128 | AI949371 | Hs. 13854 | ESTs | 9.06E−06 | 1.41 |
| 457393 | AW970603 | | gb: EST382684 MAGE resequences, MAGK Homo | 7.71E−03 | 1.40 |
| 449975 | BE546004 | Hs. 22350 | hypothetical protein LOC56757 | 8.49E−04 | 1.40 |
| 451436 | W86497 | | KIAA0729 protein | 9.49E−05 | 1.40 |
| 429490 | AI971131 | Hs23889 | ESTs, Weakly similar to ALU7_HUMAN ALU S | 2.97E−06 | 1.40 |
| 454117 | BE410100 | Hs. 40368 | adaptor-related protein complex 1, sigma | 5.93E−04 | 1.40 |
| 401431 | | | Target Exon | 1.27E−03 | 1.40 |
| 442445 | AA082665 | Hs. 209561 | KIAA1715 protein | 1.22E−04 | 1.40 |
| 410297 | AA148710 | | lumican | 3.82E−08 | 1.40 |
| 444960 | AI611317 | Hs. 341531 | ESTs | 6.84E−07 | 1.40 |
| 418835 | AL023694 | Hs. 88977 | hypothetical protein dJ511E16.2 | 1.04E−05 | 1.40 |
| 446178 | AI278844 | Hs. 149450 | ESTs | 2.18E−04 | 1.40 |
| 430299 | W28673 | Hs. 106747 | serine carboxypeptidase 1 precursor prot | 8.74E−05 | 1.40 |
| 420790 | L04791 | Hs. 99924 | excision repair cross-complementing rode | 5.78E−03 | 1.40 |
| 427701 | AA411101 | Hs. 243886 | nuclear autoantigenic sperm protein (his | 3.52E−04 | 1.40 |
| 433225 | AW816515 | Hs. 173540 | ATPase, Class V, type 10D | 9.45E−05 | 1.40 |
| 444670 | H58373 | Hs. 332938 | hypothetical protein MGC5370 | 1.04E−05 | 1.40 |
| 435099 | AC004770 | Hs. 4756 | flap structure-specific endonuclease 1 | 1.89E−03 | 1.40 |
| 446291 | BE397753 | Hs. 14623 | interferon, gamma-inducible protein 30 | 1.32E−03 | 1.40 |
| 417628 | T84214 | | gb: yd32f10.r1 Soares fetal liver spleen | 2.53E−05 | 1.40 |
| 428820 | AA436187 | Hs. 172631 | integrin, alpha M (complement component | 2.63E−06 | 1.39 |
| 438356 | AA805530 | Hs. 48527 | ESTs | 7.22E−03 | 1.39 |
| 422310 | AA316622 | Hs. 98370 | cytochrome P450, subfamily IIS, polypept | 2.06E−05 | 1.39 |
| 441149 | AI569766 | Hs. 13205 | ESTs | 2.12E−03 | 1.39 |
| 443490 | AB018313 | Hs. 9452 | KIAA0770 protein | 1.66E−07 | 1.39 |
| 425272 | AA354138 | Hs. 47209 | ESTs, Weakly similar to C35826 hypotheti | 3.57E−03 | 1.39 |
| 443037 | AW500305 | Hs. 8906 | syntaxin 7 | 1.61E−05 | 1.39 |
| 409330 | AK001231 | Hs. 53940 | hypothetical protein FLJ10369 | 9.63E−04 | 1.39 |
| 408822 | AW500715 | Hs. 57079 | Homo sapiens cDNA FLJ13267 fis, clone OV | 2.44E−06 | 1.39 |
| 426809 | BE313114 | Hs. 29706 | ESTs | 2.91E−05 | 1.39 |
| 416197 | AA176253 | Hs. 110309 | major histocompatibility complex, class | 2.96E−07 | 1.39 |
| 410334 | AW979261 | Hs. 291993 | ESTs | 5.81E−05 | 1.39 |
| 422645 | L40027 | Hs. 118890 | glycogen synthase kinase 3 alpha | 3.13E−05 | 1.39 |
| 414931 | AK000342 | Hs. 77646 | Homo sapiens mRNA; cDNA DKFZp761M0223 (f | 2.07E−04 | 1.39 |
| 434809 | AW974687 | | gb: EST386776 MAGE resequences, MAGM Homo | 6.88E−03 | 1.39 |
| 421315 | D78791 | Hs. 103419 | fasciculation and elongation protein zet | 4.60E−07 | 1.39 |
| 420985 | X94703 | | RAB28, member RAS oncogene family | 8.20E−05 | 1.39 |
| 422653 | AI092590 | Hs. 118962 | far upstream element (FUSE) binding prot | 1.20E−03 | 1.39 |
| 401047 | | | Target Exon | 5.92E−03 | 1.39 |
| 408417 | AI124541 | Hs. 44720 | hypothetical protein P1 p373c6 | 3.93E−04 | 1.39 |
| 415130 | W85893 | Hs. 249867 | ESTs | 1.44E−05 | 1.39 |
| 447511 | BE274845 | Hs. 17110 | Homo sapiens mRNA; cDNA DKFZp434C2016 (f | 9.71E−03 | 1.39 |
| 421970 | AF227156 | Hs. 110103 | RNA polymerase l transcription factor RR | 9.45E−04 | 1.38 |
| 414733 | BE514535 | Hs. 77171 | minichromosome maintenance deficient (S. | 8.98E−06 | 1.38 |
| 451107 | AA235108 | Hs. 17639 | Homo sapiens ubiquitin protein ligase (U | 1.06E−04 | 1.38 |
| 428104 | AA421350 | | ESTs | 1.62E−04 | 1.38 |
| 435124 | AA725362 | Hs. 120456 | ESTs | 2.46E−04 | 1.38 |
| 423558 | AW865643 | Hs. 275865 | ribosomal protein S18 | 7.10E−05 | 1.38 |
| 430933 | AW863635 | | gb: MR3-SN0010-270300-103-h02 SN0010 Homo | 1.88E−03 | 1.38 |
| 419511 | AA429750 | Hs. 75113 | general transcription factor IIIA | 3.02E−05 | 1.38 |
| 423787 | AJ295745 | Hs. 236204 | nuclear pore complex protein | 2.58E−04 | 1.38 |
| 438441 | AW664960 | Hs. 205319 | ESTs | 5.62E−05 | 1.38 |
| 457186 | AW371847 | Hs. 313499 | ESTs | 2.77E−04 | 1.38 |
| 444388 | AB033058 | Hs. 11101 | KIAA1232 protein | 2.86E−03 | 1.38 |
| 419135 | R61448 | Hs. 106728 | ESTs, Weakly similar to KIAA1353 protein | 1.43E−08 | 1.38 |
| 417386 | AL037228 | Hs. 82043 | D123 gene product | 7.23E−04 | 1.38 |
| 444363 | AI142827 | Hs. 143656 | ESTs | 8.10E−05 | 1.38 |
| 447829 | AI433029 | Hs. 164104 | ESTs | 7.35E−03 | 1.38 |
| 434848 | BE256304 | Hs. 32148 | AD-015 protein | 1.74E−03 | 1.38 |
| 443186 | AI885827 | Hs. 7149 | ESTs, Weakly similar to T13963 formin re | 1.89E−03 | 1.38 |
| 436563 | AJ239450 | Hs. 157874 | ESTs | 1.04E−04 | 1.38 |
| 414176 | BE140638 | Hs. 75794 | EDG-2 (endothelial differentiation, lys | 6.98E−03 | 1.38 |
| 413771 | AA810047 | Hs. 4112 | acetyl-Coenzyme A acetyltransferase 2 (a | 3.74E−04 | 1.38 |
| 430027 | AB023197 | Hs. 227743 | KIAA0980 protein | 3.32E−09 | 1.38 |
| 432403 | AA550815 | Hs. 124840 | ESTs | 1.03E−05 | 1.37 |
| 454038 | X06374 | Hs. 37040 | platelet-derived growth factor alpha pol | 2.84E−03 | 1.37 |
| 409593 | AB032985 | Hs. 55069 | neurexophilin 3 | 7.75E−04 | 1.37 |
| 408124 | U89337 | Hs. 42853 | cAMP responsive element binding protein- | 4.40E−07 | 1.37 |
| 457062 | AA404520 | Hs. 125845 | ribulose-5-phosphate-3-epimerase | 8.18E−04 | 1.37 |
| 434894 | AW977850 | Hs. 23856 | hypothetical protein MGC5297 | 4.62E−03 | 1.37 |
| 456619 | AV647917 | Hs. 107153 | inhibitor of growth family, member 1-lik | 1.49E−08 | 1.37 |
| 447811 | C15005 | Hs. 17121 | ESTs, Moderately similar to GRPM MOUSE G | 2.97E−04 | 1.37 |
| 421662 | NM_014141 | Hs. 106552 | cell recognition molecule Caspr2 | 1.32E−03 | 1.37 |
| 411678 | AI907114 | Hs. 71465 | squalene epoxidase | 7.96E−04 | 1.37 |
| 411352 | NM_002890 | Hs. 758 | RAS p21 protein activator (GTPase activa | 2.60E−05 | 1.37 |
| 420962 | NM_005904 | Hs. 100602 | MAD (mothers against decapentaplegic, Dr | 2.22E−07 | 1.37 |
| 408388 | AF091086 | Hs. 44563 | hypothetical protein | 1.02E−04 | 1.37 |
| 438517 | AI439688 | Hs. 6289 | hypothetical protein FLJ20886 | 4.50E−05 | 1.37 |
| 404813 | | | Target Exon | 1.79E−03 | 1.37 |
| 448361 | H82028 | Hs. 238707 | hypothetical protein FLJ22457 | 1.29E−04 | 1.37 |
| 441077 | AI241273 | Hs. 15312 | ESTs | 5.25E−08 | 1.36 |
| 459190 | AW410601 | Hs. 30026 | HSPC182 protein | 2.26E−04 | 1.36 |
| 421297 | AB037360 | | KIAA1255 protein | 1.33E−07 | 1.36 |
| 417929 | R27219 | Hs. 74647 | Human T-cell receptor active alpha-chain | 9.13E−05 | 1.36 |
| 421983 | AI252640 | Hs. 110364 | peptidylprolyl isomerase C (cyclophilin | 4.89E−06 | 1.36 |
| 444842 | AF084479 | Hs. 194688 | bromodomain adjacent to zinc finger doma | 1.24E−05 | 1.36 |
| 423485 | AB037809 | Hs. 129268 | KIAA1388 protein | 2.34E−03 | 1.36 |
| 446110 | AI305223 | Hs. 148056 | ESTs | 3.07E−04 | 1.36 |
| 435635 | AF220050 | Hs. 181385 | uncharacterized hematopoietic stem/proge | 1.29E−04 | 1.36 |
| 408360 | AI806090 | Hs. 44344 | hypothetical protein FLJ20534 | 1.33E−08 | 1.36 |
| 412622 | AW664708 | Hs. 171959 | ESTs | 9.40E−03 | 1.36 |
| 414718 | H95348 | Hs. 107987 | ESTs | 1.35E−03 | 1.36 |
| 444886 | AW340504 | Hs. 148557 | ESTs | 1.75E−03 | 1.36 |
| 449188 | AW072939 | Hs. 347187 | myotubularin related protein 1 | 6.45E−07 | 1.36 |
| 410804 | U64820 | Hs. 66521 | Machado-Joseph disease (spinocerebellar | 2.44E−04 | 1.36 |
| 400296 | AA305627 | Hs. 139336 | ATP-binding cassette, sub-family C (CFTR | 3.61E−03 | 1.36 |
| 427080 | AW068287 | Hs. 301175 | ras-related C3 botulinum toxin substrate | 6.15E−05 | 1.36 |
| 452934 | AA581322 | Hs. 4213 | hypothetical protein MGC16207 | 4.31E−05 | 1.36 |
| 409353 | NM_006858 | Hs. 54411 | putative T1/ST2 receptor binding protein | 7.85E−05 | 1.36 |
| 410426 | AA085039 | Hs. 285013 | putative human HLA class II associated p | 8.45E−03 | 1.36 |
| 421944 | AA344367 | Hs. 109760 | Target CAT | 5.43E−05 | 1.36 |
| 412288 | NM_003005 | Hs. 73800 | selectin P (granule membrane protein 140 | 9.21E−03 | 1.35 |
| 445165 | AV652831 | Hs. 234058 | gb: AV652831 GLC Homo sapiens cDNA clone | 2.67E−06 | 1.35 |
| 432372 | AI659280 | Hs. 155004 | ESTs | 3.61E−05 | 1.35 |
| 421887 | AW161450 | Hs. 109201 | CGI-86 protein | 9.93E−06 | 1.35 |
| 418962 | AA714835 | Hs. 271863 | ESTs | 1.92E−07 | 1.35 |
| 407885 | AA373889 | Hs. 41007 | mitochondrial ribosomal protein L22 | 4.57E−07 | 1.35 |
| 443835 | AF016371 | Hs. 9880 | peptidyl prolyl isomerase H (cyclophilin | 1.23E−06 | 1.35 |
| 456113 | H71616 | Hs. 135233 | ESTs | 2.62E−03 | 1.35 |
| 423878 | AI907090 | Hs. 52891 | hypothetical protein PRO1853 | 4.29E−05 | 1.35 |
| 434509 | AA193565 | Hs. 79276 | Homo sapiens clone IMAGE: 121558 mRNA seq | 2.52E−03 | 1.35 |
| 413129 | AF292100 | Hs. 104613 | RP42 homolog | 7.65E−06 | 1.35 |
| 419985 | H66373 | Hs. 5856 | ESTs, Highly similar to bA393J16.3 [H. sa | 8.31E−04 | 1.35 |
| 432623 | AA557351 | Hs. 152448 | ESTs, Moderately similar to S14147 multi | 7.55E−04 | 1.35 |
| 421489 | AI922821 | Hs. 32433 | ESTs | 2.40E−03 | 1.35 |
| 424539 | L02911 | Hs. 150402 | Activin A receptor, type I (ACVR1) (ALK | 5.27E−05 | 1.35 |
| 410256 | AB037732 | Hs. 61441 | KIAA1311 protein | 1.95E−05 | 1.35 |
| 410365 | AI287518 | | Homo sapiens mRNA; cDNA DKFZp586D0923 (f | 1.10E−03 | 1.35 |
| 458439 | AV647220 | Hs. 106576 | alanine-glyoxylate aminotransferase 2-li | 1.43E−05 | 1.35 |
| 406137 | | | NM_000179*: Homo sapiens mutS (E. coli) h | 1.43E−03 | 1.35 |
| 428466 | AF151063 | Hs. 184456 | hypothetical protein | 1.07E−04 | 1.35 |
| 432358 | AI093491 | Hs. 274413 | hypothetical protein FLJ11218 | 5.55E−04 | 1.35 |
| 416676 | AW392022 | Hs. 79507 | KIAA0582 protein | 2.42E−03 | 1.35 |
| 447139 | AL134744 | Hs. 10852 | ESTs | 4.56E−08 | 1.35 |
| 436137 | AI056769 | Hs. 133512 | ESTs | 9.06E−05 | 1.35 |
| 438459 | T49300 | Hs. 35304 | Homo sapiens cDNA FLJ13655 fis, clone PL | 3.65E−05 | 1.35 |
| 424649 | BE242035 | Hs. 151461 | embryonic ectoderm development | 9.97E−07 | 1.35 |
| 413806 | L12535 | Hs. 75551 | Ras suppressor protein 1 | 3.39E−05 | 1.35 |
| 424972 | BE247567 | Hs. 154045 | KIAA0082 protein | 8.45E−07 | 1.35 |
| 447387 | AI268331 | Hs. 102237 | tubby super-family protein | 1.42E−07 | 1.34 |
| 413858 | NM_001610 | Hs. 75589 | acid phosphatase 2, lysosomal | 1.36E−03 | 1.34 |
| 427399 | NM_014883 | Hs. 177664 | KIAA0914 gene product | 2.29E−06 | 1.34 |
| 455631 | BE063031 | | gb: MR0-BT0265-231199-002-e09 BT0265 Homo | 3.66E−03 | 1.34 |
| 409542 | AA503020 | Hs. 36563 | hypothetical protein FLJ22418 | 6.76E−03 | 1.34 |
| 429598 | AA811257 | Hs. 269710 | ESTs | 8.67E−05 | 1.34 |
| 421166 | AA305407 | Hs. 102308 | potassium inwardly-rectifying channel, s | 4.96E−07 | 1.34 |
| 432523 | AA551756 | | ESTs | 7.86E−06 | 1.34 |
| 445652 | AL117473 | Hs. 13036 | DKFZP727A071 protein | 2.35E−05 | 1.34 |
| 433923 | AI823453 | Hs. 146625 | ESTs | 2.49E−05 | 1.34 |
| 420843 | H96982 | Hs. 42321 | ESTs | 1.00E−03 | 1.34 |
| 440328 | AW392243 | | endogenous retroviral protease | 5.80E−04 | 1.34 |
| 442406 | NM_014846 | Hs. 8294 | KIAA0196 gene product | 5.90E−05 | 1.34 |
| 400763 | | | Target Exon | 2.50E−03 | 1.34 |
| 440973 | AI656743 | Hs. 127514 | ESTs | 9.83E−03 | 1.34 |
| 427674 | NM_003528 | Hs. 2178 | H2B histone family, member Q | 1.03E−04 | 1.34 |
| 429837 | NM_003896 | Hs. 225939 | sialyltransferase 9 (CMP-NeuAc: lactosylc | 1.21E−04 | 1.34 |
| 452887 | AI702223 | Hs. 107253 | hypothetical protein DKFZp761F241 | 1.70E−03 | 1.34 |
| 424321 | W74048 | Hs. 1765 | lymphocyte-specific protein tyrosine kin | 1.54E−05 | 1.34 |
| 412347 | AW970026 | Hs. 73818 | ubiquinol-cytochrome c reductase hinge p | 1.38E−05 | 1.34 |
| 425843 | BE313280 | Hs. 159627 | death associated protein 3 | 5.79E−05 | 1.34 |
| 412710 | AA115819 | Hs. 187593 | ESTs | 1.53E−03 | 1.34 |
| 422607 | Z45471 | Hs. 118684 | stromal cell-derived factor 2 | 1.25E−06 | 1.34 |
| 441004 | AL137488 | Hs. 7615 | Homo sapiens mRNA; cDNA DKFZp434N2030 (f | 1.10E−04 | 1.34 |
| 441229 | AW083064 | | ESTs | 8.72E−05 | 1.33 |
| 445718 | H79791 | Hs. 15227 | ESTs | 1.85E−06 | 1.33 |
| 426032 | H63477 | Hs. 81188 | ESTs, Moderately similar to ALU1_HUMAN A | 7.52E−04 | 1.33 |
| 414080 | AA135257 | Hs. 47783 | B aggressive lymphoma gene | 1.15E−04 | 1.33 |
| 407309 | AA526438 | Hs. 281680 | peroxisomal trans 2-enoyl CoA reductase; | 9.94E−06 | 1.33 |
| 409977 | AW805510 | Hs. 97056 | hypothetical protein FLJ21634 | 2.34E−04 | 1.33 |
| 447347 | AA570056 | Hs. 122730 | ESTs, Moderately similar to KIAA1215 pro | 1.70E−07 | 1.33 |
| 429682 | NM_006306 | Hs. 211602 | SMC1 (structural maintenance of chromoso | 6.55E−03 | 1.33 |
| 428023 | AL038843 | | Homo sapiens cDNA; FLJ23602 fis, clone L | 1.70E−03 | 1.33 |
| 412677 | AW029608 | Hs. 17384 | ESTs | 1.00E−03 | 1.33 |
| 424967 | AI188435 | | RAB27A, member RAS oncogene family | 5.80E−05 | 1.33 |
| 403608 | | | C3001199: gi\|7494834\|pir\|\|T15308hypothet | 1.24E−03 | 1.33 |
| 445292 | AV653264 | Hs. 13982 | Homo sapiens cDNA FLJ14666 fis, clone NT | 1.47E−04 | 1.33 |
| 425882 | U83115 | Hs. 161002 | absent in melanoma 1 | 2.79E−09 | 1.33 |
| 421767 | AW390255 | Hs. 108112 | histone fold protein CHRAC17; DNA polyme | 4.64E−05 | 1.33 |
| 439908 | AI168031 | Hs. 155507 | ESTs | 1.11E−04 | 1.33 |
| 421282 | AA286914 | Hs. 40782 | ESTs | 6.68E−06 | 1.33 |
| 420838 | AW118210 | Hs. 42321 | ESTs | 2.32E−04 | 1.33 |
| 453403 | BE466639 | Hs. 61779 | Homo sapiens cDNA FLJ13591 fis, clone PL | 4.15E−04 | 1.33 |
| 426343 | NM_014642 | Hs. 169387 | KIAA0036 gene product | 1.55E−04 | 1.33 |
| 445592 | AV654382 | Hs. 17947 | ESTs, Weakly similar to T16534 hypotheti | 1.22E−05 | 1.33 |
| 446048 | AI272364 | Hs. 182081 | KIAA1811 protein | 4.47E−03 | 1.33 |
| 415023 | AA932146 | | Homo sapiens clone TCCCIA00164 mRNA sequ | 9.58E−07 | 1.33 |
| 427726 | AI359144 | Hs. 143688 | Homo sapiens cDNA: FLJ23031 fis, clone L | 8.02E−07 | 1.33 |
| 417947 | AA323563 | Hs. 325309 | hypothetical protein FLJ14596 | 2.89E−04 | 1.33 |
| 434260 | AF121856 | Hs. 284291 | sorting nexin 6 | 2.30E−08 | 1.33 |
| 437306 | AW270552 | | ESTs | 1.55E−03 | 1.32 |
| 410125 | AK000846 | Hs. 58679 | solute carrier family 7, (cationic amino | 1.60E−04 | 1.32 |
| 451168 | D31770 | Hs. 26014 | activin A receptor, type II | 4.12E−03 | 1.32 |
| 416315 | AA179483 | Hs. 73605 | ESTs | 1.38E−05 | 1.32 |
| 411784 | AK001715 | Hs. 72085 | hypothetical protein FLJ10853 | 5.04E−08 | 1.32 |
| 452144 | AA032197 | Hs. 102558 | Homo sapiens, clone MGC: 5352, mRNA, comp | 3.29E−05 | 1.32 |
| 426012 | AA367507 | Hs. 75874 | pregnancy-associated plasma protein A | 7.29E−03 | 1.32 |
| 425295 | AA431366 | Hs. 37251 | ESTs | 4.74E−04 | 1.32 |
| 437986 | AA774575 | Hs. 121776 | testis expressed sequence 11 | 2.42E−03 | 1.32 |
| 442332 | AI693251 | Hs. 8248 | Target CAT | 1.30E−05 | 1.32 |
| 439208 | AK000299 | Hs. 180952 | dynactin 4 (p62) | 1.04E−05 | 1.32 |
| 456073 | AA587775 | Hs. 66295 | multi-PDZ-domain-containing protein | 3.40E−05 | 1.32 |
| 428738 | NM_000380 | Hs. 192803 | xeroderma pigmentosum, complementation g | 1.52E−05 | 1.32 |
| 452479 | AI903573 | | gb: RC-BT031-280199-114 BT031 Homo sapien | 5.19E−03 | 1.32 |
| 452548 | AL050321 | Hs. 301532 | CRP2 binding protein | 3.09E−06 | 1.32 |
| 450016 | AA249590 | Hs. 4747 | ESTs, Weakly similar to A28996 proline-r | 5.01E−06 | 1.32 |
| 431675 | AA699965 | | ESTs | 6.72E−04 | 1.32 |
| 430261 | AA305127 | Hs. 4147 | hypothetical protein HT023 | 4.00E−06 | 1.32 |
| 411885 | AA452636 | Hs. 131057 | ESTs, Moderately similar to CRGD_HUMAN G | 2.36E−03 | 1.32 |
| 426857 | AW581992 | Hs. 301434 | KIAA1387 protein | 7.58E−04 | 1.32 |
| 407825 | NM_006152 | Hs. 40202 | lymphoid-restricted membrane protein | 3.74E−03 | 1.32 |
| 441647 | AA534210 | Hs. 285280 | Homo sapiens cDNA: FLJ22096 fis, clone H | 5.30E−04 | 1.32 |
| 409401 | AI201895 | Hs. 181309 | proteasome (prosome, macropain) subunit, | 2.42E−05 | 1.32 |
| 437293 | AA836401 | Hs. 87860 | ESTs | 1.61E−04 | 1.32 |
| 418780 | AA228078 | Hs. 255096 | ESTs | 2.84E−03 | 1.31 |
| 431586 | AW971100 | Hs. 293189 | ESTs | 6.50E−04 | 1.31 |
| 438979 | AW976218 | Hs. 32565 | ESTs | 2.44E−06 | 1.31 |
| 458207 | T28472 | Hs. 7655 | U2 small nuclear ribonucleoprotein auxil | 1.40E−03 | 1.31 |
| 453320 | AW450240 | Hs. 257274 | ESTs | 5.85E−03 | 1.31 |
| 426796 | S78234 | Hs. 172405 | cell division cycle 27 | 1.75E−04 | 1.31 |
| 432600 | AI821085 | | gb: ns95a12.y5 NCl_CGAP_Pr3 Homo sapiens | 1.05E−03 | 1.31 |
| 409572 | AL048938 | Hs. 54971 | hypothetical protein | 1.72E−08 | 1.31 |
| 408758 | NM_003686 | Hs. 47504 | exonuclease 1 | 1.43E−04 | 1.31 |
| 445648 | AW362302 | Hs. 164090 | ESTs | 4.03E−04 | 1.31 |
| 432987 | AI864771 | Hs. 27954 | CD86 antigen (CD28 antigen ligand 2, B7- | 1.27E−06 | 1.31 |
| 419943 | AA252111 | Hs. 15200 | ESTs | 9.35E−05 | 131 |
| 413274 | NM_004893 | Hs. 75258 | H2A histone family, member Y | 5.15E−04 | 1.31 |
| 449365 | AW968261 | Hs. 118913 | ESTs, Moderately similar to T46371 hypot | 4.97E−03 | 1.31 |
| 414411 | X54079 | Hs. 76067 | heat shock 27 kD protein 1 | 1.14E−06 | 1.31 |
| 400271 | | | NM_004344: Homo sapiens centrin, EF-hand | 2.70E−04 | 1.31 |
| 448071 | BE621584 | Hs. 6983 | Homo sapiens cDNA: FLJ22646 fis, clone H | 1.50E−05 | 1.31 |
| 450289 | AI690295 | Hs. 243598 | ESTs | 6.16E−04 | 1.31 |
| 417089 | H52280 | Hs. 18612 | Homo sapiens cDNA: FLJ21909 fis, clone H | 6.27E−10 | 1.31 |
| 418876 | AA740616 | | gb: ny97f11.s1 NCI_CGAP_GCB1 Homo sapiens | 2.19E−06 | 1.31 |
| 443199 | AJ002030 | Hs. 9071 | progesterone membrane binding protein | 1.42E−06 | 1.31 |
| 411529 | AA430348 | Hs. 317596 | Homo sapiens cDNA FLJ12927 fis, clone NT | 7.08E−03 | 1.31 |
| 453407 | AW409745 | Hs. 32967 | Cockayne syndrome 1 (classical) | 2.06E−03 | 1.31 |
| 416365 | U15131 | Hs. 79265 | suppression of tumorigenicity 5 | 4.90E−04 | 1.31 |
| 440188 | AK001812 | Hs. 7036 | N-Acetylglucosamine kinase | 9.80E−05 | 1.31 |
| 440779 | AI651910 | Hs. 128556 | ESTs | 9.12E−03 | 1.31 |
| 400840 | | | Target Exon | 2.57E−05 | 1.31 |
| 431663 | NM_016569 | Hs. 267182 | TBX3-iso protein | 9.11E−05 | 1.31 |
| 409052 | AW898179 | Hs. 50123 | zinc finger protein 189 | 5.93E−05 | 1.31 |
| 446254 | BE179829 | Hs. 179852 | Homo sapiens cDNA FLJ12832 fis, clone NT | 9.61E−03 | 1.31 |
| 407177 | W88954 | Hs. 203709 | ESTs | 3.43E−03 | 1.31 |
| 417933 | X02308 | Hs. 82962 | thymidylate synthetase | 9.19E−06 | 1.31 |
| 418207 | C14685 | Hs. 34772 | ESTs | 3.94E−03 | 1.31 |
| 452139 | AA099969 | Hs. 16331 | Homo sapiens cDNA: FLJ21482 fis, clone C | 1.07E−07 | 1.31 |
| 446641 | AL049229 | Hs. 15787 | Homo sapiens mRNA; cDNA DKFZp564O1016 (f | 2.24E−03 | 1.31 |
| 407890 | AK001607 | Hs. 41127 | hypothetical protein FLJ13220 | 1.58E−06 | 1.30 |
| 417928 | AA209344 | Hs. 30177 | ESTs | 5.53E−06 | 1.30 |
| 446044 | H67567 | Hs. 13572 | calcium modulating ligand | 7.83E−09 | 1.30 |
| 431921 | N46466 | Hs. 58879 | ESTs | 2.21E−05 | 1.30 |
| 418020 | R35510 | Hs. 24990 | ESTs | 7.14E−04 | 1.30 |
| 409619 | AK001015 | Hs. 55220 | BCL2-associated athanogene 2 | 3.09E−05 | 1.30 |
| 425836 | AW955696 | Hs. 90960 | ESTs | 2.22E−06 | 1.30 |
| 444735 | BE019923 | Hs. 243122 | hypothetical protein FLJ13057 similar to | 5.41E−04 | 1.30 |
| 436535 | AW295687 | Hs. 254420 | ESTs | 1.12E−05 | 1.30 |
| 415910 | U20350 | Hs. 78913 | chemokine (C-X3-C) receptor 1 | 8.53E−05 | 1.30 |
| 445388 | AI925280 | Hs. 128777 | EST | 2.59E−06 | 1.30 |
| 448965 | AF092134 | Hs. 22679 | CGI-24 protein | 3.13E−08 | 1.30 |
| 445757 | AW449065 | Hs. 13264 | KIAA0856 protein | 7.74E−04 | 1.30 |
| 414783 | AW069569 | | unactive progesterone receptor, 23 kD | 8.35E−06 | 1.30 |
| 448244 | BE613416 | Hs. 336425 | Homo sapiens, clone MGC: 17296. mRNA, com | 3.74E−06 | 1.30 |
| 423032 | AI684746 | Hs. 119274 | RAS p21 protein activator (GTPase activa | 1.31E−04 | 1.30 |
| 437527 | AI241019 | Hs. 145644 | ESTs | 3.77E−04 | 1.30 |
| 408063 | BE086548 | Hs. 42346 | calcineurin-binding protein calsarcin-1 | 3.50E−04 | 1.30 |
| 437065 | AL036450 | Hs. 103238 | ESTs | 1.43E−03 | 1.29 |
| 446841 | AV660567 | | gb: AV660567 GLC Homo sapiens cDNA clone | 4.29E−04 | 1.29 |
| 418673 | NM_005582 | Hs. 87205 | lymphocyte antigen 64 (mouse) homolog ( | 4.79E−04 | 1.29 |
| 433671 | AW138797 | Hs. 132906 | 19A24 protein | 1.26E−03 | 1.29 |
| 420919 | M57892 | Hs. 100322 | carbonic anhydrase VI | 6.69E−04 | 1.29 |
| 447094 | X65232 | Hs. 17364 | zinc finger protein 79 (pT7) | 2.23E−04 | 1.29 |
| 417437 | U52682 | Hs. 82132 | interferon regulatory factor 4 | 9.46E−04 | 1.29 |
| 453929 | AW190054 | | gb: xl11d05.x1 NCI_CGAP_Ut4 Homo sapiens | 3.94E−03 | 1.29 |
| 417782 | T10149 | Hs. 4243 | hypothetical protein FLJ12650 | 9.78E−07 | 1.29 |
| 414438 | AI879277 | Hs. 76136 | thioredoxin | 7.32E−05 | 1.29 |
| 459436 | AA323121 | | gb: EST25881 Cerebellum II Homo sapiens c | 1.90E−04 | 1.29 |
| 424650 | AW576156 | Hs. 250824 | Homo sapiens cDNA: FLJ23435 fis. clone H | 1.85E−04 | 1.29 |
| 422843 | AA317826 | Hs. 188361 | gb: EST19817 Retina II Homo sapiens cDNA | 1.68E−04 | 1.29 |
| 458389 | H70284 | Hs. 160152 | ESTs, Weakly similar to FPHU alpha-fetop | 2.39E−06 | 1.29 |
| 443562 | AF118838 | Hs. 9599 | solute carrier family 25, member 13 (cit | 3.94E−04 | 1.29 |
| 452617 | H22717 | Hs. 7174 | hypothetical protein FLJ22759 | 5.18E−04 | 1.29 |
| 430488 | D19589 | Hs. 13453 | hypothetical protein FLJ14753 | 2.68E−04 | 1.29 |
| 413568 | AA130381 | Hs. 180257 | ESTs | 1.58E−07 | 1.29 |
| 458725 | AW970192 | Hs. 171942 | ras responsive element binding protein 1 | 2.03E−05 | 1.29 |
| 403827 | | | C4000447*: gi\|7705570\|ref\|NP_038851.1\|KI | 9.50E−03 | 1.29 |
| 440740 | H87832 | Hs. 7388 | ketch (Drosophila)-like 3 | 1.23E−05 | 1.29 |
| 410434 | AF051152 | Hs. 63668 | toll-like receptor 2 | 5.21E−04 | 1.29 |
| 409161 | W07662 | Hs. 50861 | sirtuin (silent mating type information | 5.83E−04 | 1.29 |
| 440213 | AW246253 | Hs. 7043 | succinate-CoA ligase, GDP-forming, alpha | 1.79E−08 | 1.29 |
| 443316 | AI478463 | Hs. 18443 | aldehyde dehydrogenase 8 family, member | 5.31E−05 | 1.29 |
| 436139 | AA765786 | Hs. 120936 | ESTs | 5.79E−06 | 1.29 |
| 427224 | AL135554 | | sine oculis homeobox (Drosophila) homolo | 2.33E−06 | 1.29 |
| 412520 | AA442324 | Hs. 795 | H2A histone family, member O | 1.46E−06 | 1.29 |
| 452303 | R27257 | Hs. 57734 | heat shock 70 kD protein 1B | 1.14E−04 | 1.28 |
| 420151 | AA255931 | Hs. 186704 | ESTs | 7.07E−06 | 1.28 |
| 443601 | AI078554 | Hs. 42658 | ESTs | 1.61E−05 | 1.28 |
| 450305 | AA009477 | Hs. 17572 | ESTs, Weakly similar to A46010 X-linked | 2.83E−05 | 1.28 |
| 431255 | AA497043 | Hs. 115685 | ESTs | 9.13E−03 | 1.28 |
| 419935 | AB020980 | Hs. 93832 | putative membrane protein | 1.02E−06 | 1.28 |
| 429686 | AI871613 | Hs. 28538 | Homo sapiens cDNA: FLJ21086 fis, clone C | 3.27E−03 | 1.28 |
| 442484 | AF075360 | | gb: AF075360 Human fetal liver cDNA libra | 1.79E−03 | 1.28 |
| 437916 | BE566249 | Hs. 20999 | hypothetical protein FLJ23142 | 2.64E−03 | 1.28 |
| 451789 | AW291532 | Hs. 211297 | ESTs | 9.14E−03 | 1.28 |
| 429529 | AA454190 | Hs. 24283 | ESTs, Moderately similar to reduced expr | 1.14E−03 | 1.28 |
| 434658 | AI624436 | Hs. 310286 | ESTs | 8.22E−06 | 1.28 |
| 418831 | AA488082 | | gb: ab13e05.s1 Stratagene lung (937210) H | 8.61E−04 | 1.28 |
| 429673 | AA884407 | Hs. 211595 | protein tyrosine phosphatase, non-recept | 3.23E−03 | 1.28 |
| 414430 | AI346201 | Hs. 76118 | ubiquitin carboxyl-terminal esterase L1 | 6.36E−03 | 1.28 |
| 448079 | R76981 | | thyroid hormone receptor-associated prot | 7.51E−04 | 1.28 |
| 408677 | AI279892 | Hs. 46801 | sorting nexin 14 | 4.91E−05 | 1.28 |
| 456508 | AA502764 | Hs. 123469 | ESTs, Weakly similar to AF208855 1 BM-01 | 8.25E−05 | 1.28 |
| 421290 | NM_014368 | Hs. 103137 | LIM homeobox protein 6 | 3.64E−04 | 1.28 |
| 431829 | BE467737 | Hs. 146125 | ESTs | 1.95E−03 | 1.28 |
| 431892 | AA521315 | Hs. 194424 | ESTs | 5.86E−03 | 1.28 |
| 425587 | AB018332 | Hs. 158319 | KIAA0789 gene product | 1.52E−03 | 1.28 |
| 428248 | AI126772 | Hs. 40479 | ESTs | 4.53E−03 | 1.28 |
| 428580 | AF118452 | Hs. 184945 | gastrulation brain homeo box 2 | 2.37E−03 | 1.28 |
| 447023 | AA356764 | Hs. 17109 | integral membrane protein 2A | 2.36E−05 | 1.28 |
| 442287 | AW952703 | Hs. 8182 | synaptic nuclei expressed gene 1b | 6.33E−05 | 1.28 |
| 421215 | AI868634 | Hs. 246358 | ESTs, Weakly similar to T32250 hypotheti | 2.84E−06 | 1.28 |
| 410383 | AA084561 | Hs. 151093 | hypothetical protein DKFZp434G1415 | 9.07E−04 | 1.28 |
| 446832 | NM_006314 | Hs. 16232 | connector enhancer of KSR-like (Drosophi | 1.67E−03 | 1.28 |
| 453648 | W21493 | Hs. 28329 | hypothetical protein FLJ14005 | 3.53E−05 | 1.27 |
| 450869 | R05747 | | ESTs, Weakly similar to organic anion tr | 4.55E−05 | 1.27 |
| 429722 | U48629 | Hs. 12731 | ESTs | 3.48E−03 | 1.27 |
| 427247 | AW504221 | Hs. 174103 | integrin, alpha L (antigen CD11A (p180). | 7.71E−03 | 1.27 |
| 415276 | U88666 | Hs. 78353 | SFRS protein kinase 2 | 2.57E−05 | 1.27 |
| 437406 | AL390182 | | Homo sapiens mRNA full length insert cDN | 8.76E−04 | 1.27 |
| 451743 | AW074266 | Hs. 23071 | ESTs | 3.36E−06 | 1.27 |
| 429379 | NM_014840 | Hs. 200598 | KIAA0537 gene product | 2.54E−03 | 1.27 |
| 409205 | AI952884 | Hs. 14832 | ESTs, Moderately similar to unnamed prot | 4.80E−04 | 1.27 |
| 433590 | N98410 | Hs. 48364 | Homo sapiens regulator of G-protein sign | 2.79E−07 | 1.27 |
| 411096 | U80034 | Hs. 68583 | mitochondrial intermediate peptidase | 5.65E−03 | 1.27 |
| 433658 | L03678 | Hs. 156110 | immunoglobulin kappa constant | 1.89E−03 | 1.27 |
| 411202 | AW163223 | Hs. 315482 | ESTs, Weakly similar to I38022 hypotheti | 7.62E−06 | 1.27 |
| 437612 | AA827715 | Hs. 105153 | Homo sapiens, clone IMAGE: 3461987, mRNA, | 5.16E−03 | 1.27 |
| 450459 | AI697193 | Hs. 299254 | Homo sapiens cDNA: FLJ23597 fis, clone L | 1.63E−03 | 1.27 |
| 458172 | BE007237 | | gb: PM0-BN0139-050500-003-g09 BN0139 Homo | 6.27E−04 | 1.27 |
| 437543 | H16443 | Hs. 7117 | glutamate receptor, ionotropic, AMPA 1 | 6.23E−08 | 1.27 |
| 430958 | M81829 | Hs. 248160 | somatostatin receptor 1 | 6.82E−06 | 1.27 |
| 408196 | AL034548 | Hs. 43627 | SRY (sex determining region Y)-box 22 | 1.40E−05 | 1.27 |
| 422385 | BE549407 | Hs. 115823 | ribonuclease P, 40 kD subunit | 6.73E−05 | 1.27 |
| 449736 | R50120 | Hs. 25367 | ESTs, Weakly similar to ALR H. sapiens | 1.37E−03 | 1.27 |
| 414992 | D63302 | | gb: HUM521A10B Clontech human placenta po | 4.14E−06 | 1.27 |
| 438847 | AA827232 | Hs. 245362 | ESTs | 1.91E−03 | 1.27 |
| 447445 | AW169369 | Hs. 158936 | ESTs | 1.04E−03 | 1.27 |
| 452386 | R12499 | Hs. 20468 | ESTs | 1.20E−04 | 1.27 |
| 410215 | AI694353 | | ESTs, Weakly similar to AF205600 1 trans | 9.81E−03 | 1.27 |
| 411115 | AW818453 | | gb: RC1-ST0278-020300-015-d01 ST0278 Homo | 4.06E−03 | 1.27 |
| 445589 | AI565993 | Hs. 222189 | ESTs | 5.35E−05 | 1.27 |
| 433689 | AI802331 | Hs. 98289 | ESTs, Weakly similar to I38022 hypotheti | 2.84E−03 | 1.27 |
| 449704 | AK000733 | Hs. 23900 | GTPase activating protein | 2.01E−05 | 1.27 |
| 407604 | AW191962 | | collagen, type VIII, alpha 2 | 6.07E−03 | 1.27 |
| 453985 | N44545 | Hs. 251865 | ESTs | 3.21E−05 | 1.27 |
| 409652 | BE208589 | Hs. 55601 | host cell factor 2 | 1.24E−04 | 1.26 |
| 403809 | | | NM_024743*: Homo sapiens hypothetical pro | 2.01E−03 | 1.26 |
| 417798 | Y09858 | Hs. 82577 | spindlin-like | 1.17E−03 | 1.26 |
| 445813 | Z42023 | Hs. 106576 | alanine-glyoxylate aminotransferase 2-li | 5.56E−06 | 1.26 |
| 447769 | AW873704 | Hs. 320831 | Homo sapiens cDNA FLJ14597 fis, clone NT | 1.34E−05 | 1.26 |
| 451593 | AF151879 | Hs. 26706 | CGI-121 protein | 9.80E−06 | 1.26 |
| 452780 | BE171598 | | ESTs, Weakly similar to I38022 hypotheti | 5.91E−07 | 1.26 |
| 438032 | BE045624 | Hs. 152992 | ESTs | 2.14E−03 | 1.26 |
| 426925 | NM_001196 | Hs. 315689 | Homo sapiens cDNA: FLJ22373 fis, clone H | 5.81E−07 | 1.26 |
| 445870 | AW410053 | Hs. 13406 | syntaxin 18 | 7.80E−10 | 1.26 |
| 422043 | AL133649 | Hs. 110953 | retinoic acid induced 1 | 4.15E−04 | 1.26 |
| 403142 | | | NM_002706*: Homo sapiens protein phosphat | 8.57E−05 | 1.26 |
| 437109 | AW006781 | Hs. 5457 | hypothetical protein FLJ10738 | 2.23E−06 | 1.26 |
| 443551 | AI654356 | Hs. 272792 | hypothetical protein FLJ20307 | 1.53E−06 | 1.26 |
| 439161 | Y15164 | Hs. 6483 | oral-facial-digital syndrome 1 | 3.38E−04 | 1.26 |
| 411143 | AI525989 | Hs. 9088 | hypothetical protein MGC3200 | 4.14E−04 | 1.26 |
| 449082 | BE387561 | Hs. 22981 | DKFZP586M1523 protein | 2.46E−06 | 1.26 |
| 441700 | AA233556 | Hs. 126908 | hypothetical protein FLJ12994 | 7.57E−03 | 1.26 |
| 402813 | | | ENSP00000236495*: DJ1024N4.1 (novel Sodiu | 4.14E−03 | 1.26 |
| 440043 | BE277457 | Hs. 30661 | hypothetical protein MGC4606 | 7.22E−05 | 1.26 |
| 427629 | AA233210 | Hs. 179943 | ribosomal protein L11 | 6.93E−04 | 1.26 |
| 437231 | BE303000 | Hs. 288929 | hypothetical protein FLJ13258 similar to | 8.51E−05 | 1.25 |
| 433043 | W57554 | Hs. 125019 | lymphoid nuclear protein (LAF-4) mRNA | 1.39E−04 | 1.25 |
| 408088 | AW157022 | Hs. 343551 | hypothetical protein FLJ22584 | 6.02E−05 | 1.25 |
| 423598 | BE247600 | Hs. 155538 | ESTs | 1.11E−03 | 1.25 |
| 452767 | AW014195 | Hs. 61472 | ESTs, Weakly similar to YAE6_YEAST HYPOT | 9.61E−06 | 1.25 |
| 459245 | BE242623 | Hs. 31939 | manic fringe (Drosophila) homolog | 1.38E−03 | 1.25 |
| 448984 | AW751955 | Hs. 22753 | hypothetical protein FLJ22318 | 3.98E−04 | 1.25 |
| 409513 | AW966728 | Hs. 54642 | methionine adenosyltransferase II, beta | 1.85E−06 | 1.25 |
| 425508 | AA991551 | Hs. 97013 | Homo sapiens, Similar to RIKEN cDNA 2310 | 4.63E−05 | 1.25 |
| 408348 | AW269752 | Hs. 249828 | ESTs | 2.09E−03 | 1.25 |
| 407783 | AW996872 | Hs. 172028 | a disintegrin and metalloproteinase doma | 4.38E−04 | 1.25 |
| 451188 | AA054214 | Hs. 60684 | ESTs | 7.70E−03 | 1.25 |
| 458311 | AF069478 | | gb: AF069478 Homo sapiens astrocytoma lib | 3.91E−04 | 1.25 |
| 439747 | AK001148 | Hs. 6671 | COP9 complex subunit 4 | 1.38E−05 | 1.25 |
| 422481 | AL050163 | Hs. 117339 | DNAX-activation protein 10 | 1.87E−03 | 1.25 |
| 417954 | AI633943 | Hs. 26613 | ESTs, Weakly similar to no similarities | 1.54E−03 | 1.25 |
| 417831 | H16423 | Hs. 82685 | CD47 antigen (Rh-related antigen, integr | 7.34E−07 | 1.25 |
| 411252 | AB018549 | Hs. 69328 | MD-2 protein | 1.16E−08 | 1.25 |
| 403538 | | | Target Exon | 4.75E−03 | 1.25 |
| 424464 | R68537 | Hs. 17962 | ESTs | 9.59E−05 | 1.25 |
| 413533 | BE146973 | | gb: QV4-HT0222-011199-019-e05 HT0222 Homo | 6.62E−07 | 1.25 |
| 416959 | D28459 | Hs. 80612 | ubiquitin-conjugating enzyme E2A (RAD6 h | 2.81E−05 | 1.25 |
| 445715 | AB012958 | Hs. 13137 | UV radiation resistance associated gene | 1.01E−05 | 1.25 |
| 408738 | NM_014785 | Hs. 47313 | KIAA0258 gene product | 3.07E−05 | 1.25 |
| 424931 | BE244070 | Hs. 1845 | MHC class 1 region ORF | 2.06E−07 | 1.24 |
| 431118 | BE264901 | Hs. 250502 | carbonic anhydrase VIII | 4.32E−03 | 1.24 |
| 450196 | AW956868 | Hs. 24608 | DKFZP564D177 protein | 1.20E−08 | 1.24 |
| 452537 | AW247390 | Hs. 77735 | hypothetical protein FLJ11618 | 9.35E−04 | 1.24 |
| 439653 | AW021103 | Hs. 6631 | hypothetical protein FLJ20373 | 8.13E−04 | 1.24 |
| 407827 | BE278431 | Hs. 40323 | BUB3 (budding uninhibited by benzimidazo | 4.90E−05 | 1.24 |
| 457300 | AW297436 | Hs. 40328 | Homo sapiens cDNA: FLJ21663 fis, clone C | 2.26E−04 | 1.24 |
| 425575 | AB012851 | Hs. 158311 | Musashi (Drosophila) homolog 1 | 7.90E−03 | 1.24 |
| 442410 | AW996503 | Hs. 197680 | ESTs | 6.94E−03 | 1.24 |
| 402969 | | | ENSP00000217067*: DJ781B1.3 (A novel prot | 7.49E−05 | 1.24 |
| 438899 | AF085833 | Hs. 135624 | ESTs | 3.90E−03 | 1.24 |
| 416877 | BE386266 | Hs. 85658 | hypothetical protein FLJ23436 | 1.12E−05 | 1.24 |
| 434664 | R98754 | Hs. 271119 | ESTs | 5.52E−03 | 1.24 |
| 423846 | AW978332 | Hs. 133260 | hypothetical protein FLJ20354 | 5.39E−03 | 1.24 |
| 442952 | AI743261 | Hs. 131860 | ESTs | 4.40E−03 | 1.24 |
| 432201 | AI538613 | Hs. 298241 | Transmembrane protease, serine 3 | 2.00E−04 | 1.24 |
| 439921 | AL110209 | Hs. 6770 | LCAT-like lysophospholipase | 4.54E−06 | 1.24 |
| 431183 | NM_006855 | Hs. 250696 | KDEL (Lys-Asp-Glu-leu) endoplasmic retic | 2.31E−03 | 1.24 |
| 407705 | AB023139 | Hs. 37892 | KIAA0922 protein | 8.97E−06 | 1.24 |
| 406182 | | | Target Exon | 3.52E−03 | 1.24 |
| 433394 | AI907753 | Hs. 93810 | cerebral cavernous malformations 1 | 5.10E−06 | 1.24 |
| 451687 | AL041260 | Hs. 26837 | Homo sapiens mRNA; cDNA DKFZp586K1123 (f | 1.55E−04 | 1.24 |
| 456497 | AW967956 | Hs. 123648 | ESTs, Weakly similar to AF108460 1 ubinu | 1.58E−04 | 1.24 |
| 408392 | U28831 | Hs. 44566 | KIAA1641 protein | 1.52E−03 | 1.24 |
| 446501 | AI302616 | | ESTs | 1.06E−04 | 1.24 |
| 456023 | R00028 | Hs. 274344 | gb: ye70a06.s1 Soares fetal liver spleen | 8.92E−04 | 1.24 |
| 433215 | AB040912 | Hs. 191098 | hypothetical protein FLJ11598 | 1.73E−04 | 1.24 |
| 415261 | T40928 | Hs. 8346 | ESTs | 4.69E−04 | 1.24 |
| 414408 | BE294783 | Hs. 279497 | hypothetical protein MGC4638 | 3.34E−04 | 1.24 |
| 407478 | L77559 | | gb: Homo sapiens DGS-B partial mRNA. | 6.01E−03 | 1.24 |
| 406364 | | | Target Exon | 2.36E−03 | 1.24 |
| 445006 | W91903 | Hs. 124814 | ESTs | 1.24E−03 | 1.24 |
| 411821 | BE299339 | Hs. 72249 | three-PDZ containing protein similar to | 1.81E−05 | 1.24 |
| 430275 | Z11773 | Hs. 237786 | zinc finger protein 187 | 1.54E−03 | 1.24 |
| 445932 | BE046441 | Hs. 333555 | Homo sapiens clone 24859 mRNA sequence | 1.72E−03 | 1.24 |
| 409324 | W76202 | Hs. 9280 | lipoic acid synthetase | 8.62E−04 | 1.24 |
| 450371 | R34926 | | son of sevenless (Drosophila) homolog 1 | 2.33E−04 | 1.24 |
| 419309 | AW574793 | Hs. 321687 | Homo sapiens. Similar to F-box only prot | 8.89E−04 | 1.24 |
| 436343 | H08024 | Hs. 5148 | FLN29 gene product | 1.34E−05 | 1.24 |
| 413654 | AA331881 | Hs. 75454 | peroxiredoxin 3 | 1.24E−04 | 1.23 |
| 420776 | W38672 | Hs. 184067 | ESTs | 8.11E−04 | 1.23 |
| 414496 | W73853 | | ESTs | 3.85E−04 | 1.23 |
| 434521 | NM_002267 | Hs. 3886 | karyopherin alpha 3 (importin alpha 4) | 5.40E−05 | 1.23 |
| 434158 | T86534 | Hs. 14372 | ESTs | 6.53E−04 | 1.23 |
| 430367 | AW607425 | Hs. 239938 | hypothetical protein | 1.32E−03 | 1.23 |
| 447783 | AF054178 | Hs. 19561 | NM_005001: Homo sapiens NADH dehydrogenas | 2.94E−04 | 1.23 |
| 459697 | AA406062 | Hs. 98002 | ESTs | 1.09E−03 | 1.23 |
| 434519 | AA635727 | Hs. 136581 | ESTs, Weakly similar to ARNO_HUMAN ARF N | 1.26E−04 | 1.23 |
| 449843 | R85337 | Hs. 24030 | solute carrier family 31 (copper transpo | 1.12E−05 | 1.23 |
| 457000 | NM_006750 | Hs. 172278 | syntrophin, beta 2 (dystrophin-associate | 3.72E−03 | 1.23 |
| 423743 | AB023148 | Hs. 173373 | KIAA0931 protein | 1.13E−03 | 1.23 |
| 457231 | AI472022 | Hs. 301959 | proline synthetase co-transcribed (bacte | 8.57E−05 | 1.23 |
| 433192 | AB040880 | Hs. 98968 | ESTs | 8.41E−05 | 1.23 |
| 444636 | T96667 | Hs. 17877 | ESTs | 8.18E−07 | 1.23 |
| 430289 | AK001952 | Hs. 238039 | hypothetical protein FLJ11090 | 5.85E−03 | 1.23 |
| 459647 | R34107 | Hs. 321450 | pregnancy specific beta-1-glycoprotein 1 | 1.04E−04 | 1.23 |
| 427729 | AB033100 | Hs. 300646 | KIAA1274 protein (similar to mouse palad | 1.97E−03 | 1.23 |
| 414884 | R54418 | Hs. 183745 | hypothetical protein FLJ13456 | 7.32E−07 | 1.23 |
| 448554 | NM_016169 | Hs. 21431 | suppressor of fused | 2.82E−06 | 1.23 |
| 413116 | AA127161 | Hs. 4783 | hypothetical protein FLJ22035 | 6.43E−05 | 1.23 |
| 453563 | AW608906. comp | Hs. 181163 | hypothetical protein MGC5629 | 9.15E−03 | 1.23 |
| 407955 | BE536739 | Hs. 109909 | ESTs | 3.61E−04 | 1.23 |
| 405232 | | | NM_015832: Homo sapiens methyl-CpG bindin | 2.82E−03 | 1.23 |
| 457634 | AA609738 | Hs. 16525 | ESTs | 5.66E−05 | 1.23 |
| 455100 | BE160198 | | gb: QV1-HT0413-010200-059-h03 HT0413 Homo | 2.91E−03 | 1.23 |
| 419424 | BE041820 | Hs. 38516 | Homo sapiens, clone MGC: 15887, mRNA, com | 4.91E−04 | 1.23 |
| 408770 | AW270608 | Hs. 170195 | bone morphogenetic protein 7 (osteogenic | 1.86E−03 | 1.23 |
| 433854 | AA610649 | Hs. 333239 | ESTs | 5.52E−05 | 1.23 |
| 440453 | AA885523 | Hs. 157003 | ESTs | 6.78E−03 | 1.23 |
| 439452 | AA918317 | Hs. 57987 | B-cell CLL/lymphoma 11B (zinc finger pro | 3.39E−03 | 1.22 |
| 403417 | | | C18000266*: gi\|7243027\|dbj\|BAA92561.1\|(A | 5.34E−03 | 1.22 |
| 452582 | AL137407 | Hs. 29911 | Homo sapiens mRNA; cDNA DKFZp434M232 (fr | 4.03E−04 | 1.22 |
| 410043 | D30612 | Hs. 58167 | zinc finger protein 282 | 1.77E−06 | 1.22 |
| 405257 | | | Target Exon | 1.89E−03 | 1.22 |
| 441869 | NM_003947 | Hs. 8004 | huntingtin-associated protein interactin | 7.73E−03 | 1.22 |
| 442103 | AA333367 | Hs. 8088 | similar to S. cerevisiae Sec6p and R. no | 3.85E−03 | 1.22 |
| 450256 | AA286887 | Hs. 24724 | MFH-amplified sequences with leucine−ric | 7.06E−07 | 1.22 |
| 444693 | AB002327 | Hs. 11711 | KIAA0329 gene product | 8.55E−05 | 1.22 |
| 429574 | BE268321 | Hs. 208912 | hypothetical protein MGC861 | 5.48E−03 | 1.22 |
| 436906 | H95990 | Hs. 181244 | major histocompatibility complex, class | 7.11E−06 | 1.22 |
| 444476 | AF020038 | Hs. 11223 | isocitrate dehydrogenase 1 (NADP), solub | 2.03E−03 | 1.22 |
| 438393 | AA351815 | Hs. 50740 | Homo sapiens cDNA: FLJ22272 fis, clone H | 3.30E−08 | 1.22 |
| 422267 | AB033044 | Hs. 114012 | KIAA1218 protein | 1.57E−05 | 1.22 |
| 436796 | BE515260 | Hs. 5320 | hypothetical protein | 9.92E−05 | 1.22 |
| 431328 | AA502999 | Hs. 291591 | ESTs | 1.24E−04 | 1.22 |
| 450273 | AW296454 | Hs. 24743 | hypothetical protein FLJ20171 | 6.29E−03 | 1.22 |
| 457284 | AF102850 | Hs. 227933 | Alg5, S. cerevisiae, homolog of | 3.22E−05 | 1.22 |
| 417767 | BE242241 | Hs. 82542 | acyloxyacyl hydrolase (neutrophil) | 1.33E−05 | 1.22 |
| 438010 | BE243045 | Hs. 5990 | tetratricopeptide repeat domain 4 | 1.84E−06 | 1.22 |
| 408981 | AW500797 | Hs. 49427 | Gem-interacting protein | 1.50E−05 | 1.22 |
| 403585 | | | Target Exon | 6.89E−05 | 1.22 |
| 433556 | W56321 | Hs. 111460 | calcium/calmodulin-dependent protein kin | 2.49E−06 | 1.22 |
| 418077 | AA112036 | Hs. 83419 | KIAA0252 protein | 8.72E−05 | 1.22 |
| 411806 | AW864547 | | gb: PM4-SN0016-120500-003-b12 SN0016 Homo | 9.46E−04 | 1.22 |
| 452664 | AA398859 | Hs. 18397 | hypothetical protein FLJ23221 | 1.42E−08 | 1.22 |
| 432458 | AI968598 | Hs. 78768 | malignant cell expression-enhanced gene/ | 9.31E−07 | 1.22 |
| 453122 | AI953717 | Hs. 232214 | ESTs | 3.42E−04 | 1.22 |
| 424914 | AA348410 | Hs. 119065 | ESTs | 3.37E−04 | 1.22 |
| 412633 | AF001691 | Hs. 74304 | periplakin | 2.95E−04 | 1.21 |
| 451254 | AI571016 | Hs. 172967 | ESTs | 6.90E−04 | 1.21 |
| 439136 | AA830610 | Hs. 209011 | ESTs | 1.64E−03 | 1.21 |
| 420231 | R06866 | Hs. 19813 | ESTs | 4.37E−04 | 1.21 |
| 428720 | T90468 | Hs. 178154 | ESTs | 7.33E−04 | 1.21 |
| 452032 | BE244005 | Hs. 27610 | retinoic acid-and interferon-inducible | 3.55E−05 | 1.21 |
| 422032 | AA476966 | Hs. 110857 | polymerase (RNA) III (DNA directed) poly | 6.42E−03 | 1.21 |
| 430107 | AA465293 | Hs. 105069 | ESTs | 2.55E−04 | 1.21 |
| 442202 | BE272862 | Hs. 106534 | hypothetical protein FLJ22625 | 1.28E−05 | 1.21 |
| 429012 | AW629596 | Hs. 194726 | BCL2-associated athanogene 4 | 9.71E−05 | 1.21 |
| 432676 | AI187366 | | gb: qf29c01.x1 Soares_testis_NHT Homo sap | 2.09E−05 | 1.21 |
| 407946 | AA226495 | Hs. 154292 | ESTs | 2.27E−05 | 1.21 |
| 458216 | AW024282 | Hs. 104938 | hypothetical protein MGC15906 | 5.72E−07 | 1.21 |
| 403832 | | | C4000013*: gi\|11427234\|ref\|XP_009399.1\|z | 2.50E−03 | 1.21 |
| 448334 | AA767011 | Hs. 119960 | DKFZP727G051 protein | 6.83E−03 | 1.21 |
| 437378 | AI198823 | Hs. 160473 | ESTs | 7.48E−04 | 1.21 |
| 425604 | U94320 | Hs. 158330 | neuropeptide Y receptor Y5 | 5.70E−05 | 1.21 |
| 423024 | AA593731 | Hs. 325823 | ESTs, Moderately similar to ALU5_HUMAN A | 6.88E−04 | 1.21 |
| 423609 | AA328348 | Hs. 218289 | ESTs | 5.96E−04 | 1.21 |
| 439317 | AF086127 | Hs. 50600 | ESTs, Weakly similar to T47156 hypotheti | 1.32E−04 | 1.21 |
| 441765 | AI474058 | Hs. 170514 | ESTs | 5.09E−03 | 1.21 |
| 425251 | Z22521 | Hs. 155342 | protein kinase C, delta | 3.47E−07 | 1.21 |
| 452185 | AA076467 | Hs. 246218 | Homo sapiens cDNA: FLJ21781 fis, clone H | 2.31E−03 | 1.21 |
| 433018 | AI669760 | Hs. 188881 | ESTs | 4.27E−05 | 1.21 |
| 450622 | AI660285 | Hs. 58210 | ESTs, Highly similar to ITH4_HUMAN INTER | 1.13E−04 | 1.21 |
| 453314 | R05604 | Hs. 19507 | ESTs | 9.85E−03 | 1.21 |
| 431375 | AI767882 | Hs. 264347 | ESTs | 1.96E−05 | 1.21 |
| 435521 | W23814 | Hs. 6361 | mitogen-activated protein kinase kinase | 6.96E−07 | 1.21 |
| 422642 | AA314041 | | gb: EST185894 Colon carcinoma (HCC) cell | 2.63E−03 | 1.21 |
| 429855 | AW385597 | Hs. 199630 | ESTs, Weakly similar to B34087 hypotheti | 8.01E−03 | 1.21 |
| 412141 | AI183838 | Hs. 48938 | hypothetical protein FLJ21802 | 2.02E−03 | 1.21 |
| 423799 | AW026300 | Hs. 132906 | 19A24 protein | 6.14E−03 | 1.21 |
| 432377 | AA534670 | | ESTs, Weakly similar to 2004399A chromos | 2.76E−03 | 1.21 |
| 450663 | H43540 | Hs. 25292 | ribonuclease HI, large subunit | 2.34E−03 | 1.21 |
| 435561 | AA351978 | Hs. 4943 | hepatocellular carcinoma associated prot | 9.96E−04 | 1.21 |
| 415660 | AI909007 | Hs. 78563 | ubiquitin-conjugating enzyme E2G 1 (homo | 4.03E−04 | 1.21 |
| 432387 | BE094222 | Hs. 274457 | Homo sapiens mRNA; cDNA DKFZp434H2226 (f | 1.53E−03 | 1.21 |
| 458443 | AV647010 | Hs. 27 | glycine dehydrogenase (decarboxylating; | 9.56E−04 | 1.21 |
| 442281 | N34742 | Hs. 170065 | Homo sapiens cDNA FLJ13492 fis, clone PL | 6.38E−06 | 1.21 |
| 428553 | AA181641 | Hs. 184907 | G protein-coupled receptor 1 | 3.19E−04 | 1.21 |
| 431604 | AF175265 | Hs. 264190 | vacuolar protein sorting 35 (yeast homol | 1.69E−05 | 1.20 |
| 458468 | AV648170 | Hs. 58756 | ESTs | 8.67E−04 | 1.20 |
| 427546 | AA405280 | Hs. 36793 | hypothetical protein FLJ23188 | 2.59E−04 | 1.20 |
| 402964 | | | NM_022095*: Homo sapiens hypothetical C2H | 1.55E−04 | 1.20 |
| 442818 | AK001741 | Hs. 8739 | hypothetical protein FLJ10879 | 3.66E−03 | 1.20 |
| 417957 | H53497 | Hs. 83006 | CGI-139 protein | 2.93E−06 | 1.20 |
| 445298 | AW295700 | | ESTs | 1.03E−03 | 1.20 |
| 437594 | AA761431 | Hs. 74335 | heat shock 90 kD protein 1, beta | 1.32E−03 | 1.20 |
| 414704 | NM_014757 | Hs. 76986 | mastermind (Drosophila), homolog of | 3.10E−03 | 1.20 |
| 420339 | AW968259 | Hs. 186647 | ESTs | 6.90E−06 | 1.20 |
| 436754 | AI061288 | Hs. 133437 | ESTs | 3.43E−03 | 1.20 |
| 422798 | R92347 | Hs. 34574 | ESTs, Weakly similar to ALU1_HUMAN ALU S | 254E−03 | 1.20 |
| 444059 | R69743 | Hs. 116774 | integrin, alpha 1 | 9.70E−06 | 1.20 |
| 433032 | AI084066 | Hs. 20072 | myosin regulatory light chain interactin | 9.91E−06 | 1.20 |
| 408761 | AA057264 | Hs. 238936 | ESTs, Weakly similar to (defline not ava | 3.30E−07 | 1.20 |
| 443791 | N64458 | Hs. 143345 | ESTs | 1.29E−05 | 1.20 |
| 434359 | AF129536 | Hs. 284226 | F-box only protein 6 | 6.18E−06 | 1.20 |
| 413277 | H24177 | Hs. 75262 | cathepsin O | 1.91E−07 | 1.20 |
| 445464 | AW172389 | Hs. 249999 | ESTs | 1.75E−03 | 1.20 |
| 401060 | | | Target Exon | 3.25E−05 | 1.20 |
| 407588 | AW969534 | Hs. 267905 | hypothetical protein FLJ10422 | 1.12E−06 | 1.20 |
| 415245 | N59650 | Hs. 27252 | ESTs | 1.05E−03 | 1.20 |
| 456577 | BE246444 | Hs. 283685 | hypothetical protein FLJ20396 | 2.94E−03 | 1.20 |
| 456761 | D59899 | Hs. 127842 | CGI-142 | 8.55E−03 | 1.20 |
| 446868 | AV660737 | | ESTs | 6.71E−03 | 1.20 |
| 421506 | BE302796 | Hs. 105097 | thymidine kinase 1, soluble | 2.57E−04 | 1.20 |
| 426181 | AA371422 | Hs. 334371 | hypothetical protein MGC13096 | 6.57E−05 | 1.20 |
| 417177 | NM_004458 | Hs. 81452 | fatty-acid-Coenzyme A ligase, long-chain | 1.65E−04 | 1.20 |
| 425199 | R97184 | | gb: yq58f12.r1 Soares fetal liver spleen | 6.03E−03 | 1.20 |
| 414709 | AA704703 | Hs. 77031 | Sp2 transcription factor | 1.06E−04 | 1.20 |
| 406350 | | | Target Exon | 5.04E−03 | 1.20 |
| 418253 | AA215539 | Hs. 283643 | Homo sapiens cDNA FLJ11606 fis, clone HE | 1.06E−04 | 1.20 |
| 419367 | AW132011 | Hs. 118236 | Homo sapiens cDNA: FLJ22132 fis, clone H | 3.41E−04 | 1.19 |
| 420517 | AB011115 | Hs. 98507 | KIAA0543 protein | 1.61E−04 | 1.19 |
| 432441 | AW292425 | Hs. 163484 | ESTs | 6.62E−05 | 1.19 |
| 434163 | AW974720 | Hs. 25206 | group XII secreted phospholipase A2 | 2.63E−03 | 1.19 |
| 408688 | AI634522 | Hs. 152925 | KIAA1268 protein | 4.18E−05 | 1.19 |
| 434560 | R13052 | Hs. 3964 | Homo sapiens clone 24877 mRNA sequence | 6.15E−03 | 1.19 |
| 420468 | NM_006980 | Hs. 97996 | transcription termination factor, mitoch | 5.07E−04 | 1.19 |
| 409646 | AW161391 | Hs. 709 | deoxycytidine kinase | 8.94E−05 | 1.19 |
| 431234 | AL389985 | Hs. 301637 | zinc finger protein 258 | 6.12E−04 | 1.19 |
| 449259 | AW452058 | Hs. 257519 | ESTs | 6.09E−05 | 1.19 |
| 420104 | U09825 | Hs. 1287 | zinc finger protein 173 | 3.24E−08 | 1.19 |
| 436692 | AW243158 | Hs. 5297 | DKFZP564A2416 protein | 6.42E−10 | 1.19 |
| 452715 | Z21093 | Hs. 30352 | ribosomal protein S6 kinase, 52 kD, polyp | 8.38E−06 | 1.19 |
| 412372 | R65998 | Hs. 285243 | hypothetical protein FLJ22029 | 8.03E−04 | 1.19 |
| 452007 | AA426234 | Hs. 34906 | ESTs, Weakly similar to T17210 hypotheti | 1.59E−07 | 1.19 |
| 437943 | NM_016353 | Hs. 5943 | rec | 6.25E−05 | 1.19 |
| 452983 | L32140 | Hs. 531 | afamin | 8.93E−06 | 1.19 |
| 422283 | AW411307 | Hs. 114311 | CDC45 (cell division cycle 45, S.cerevis | 2.07E−04 | 1.19 |
| 422061 | BE178434 | Hs. 267995 | ESTs, Moderately similar to G02654 ribos | 2.33E−03 | 1.19 |
| 439318 | AW837046 | Hs. 6527 | G protein-coupled receptor 56 | 4.41E−03 | 1.19 |
| 428972 | AK001470 | Hs. 194692 | cysteine desulfurase | 5.56E−03 | 1.19 |
| 418384 | AW149266 | Hs. 25130 | Homo sapiens cDNA FLJ14923 fis, clone PL | 1.66E−03 | 1.19 |
| 409442 | AA310162 | Hs. 169248 | cytochrome c | 8.53E−05 | 1.19 |
| 411299 | BE409857 | Hs. 69499 | hypothetical protein | 1.40E−06 | 1.19 |
| 441307 | AW071696 | Hs. 209065 | hypothetical protein FLJ14225 | 1.90E−06 | 1.19 |
| 421413 | AI826128 | Hs. 55209 | ESTs, Weakly similar to A49364 59 protei | 1.64E−04 | 1.19 |
| 418560 | AW291332 | Hs. 194414 | ESTs | 9.68E−03 | 1.19 |
| 428873 | AI701609 | Hs. 98908 | ESTs | 8.52E−03 | 1.19 |
| 433330 | AW207084 | Hs. 132816 | hypothetical protein MGC14801 | 1.55E−04 | 1.19 |
| 424212 | NM_005814 | Hs. 143131 | glycoprotein A33 (transmembrane) | 2.16E−03 | 1.19 |
| 444445 | AA342329 | Hs. 115920 | Homo sapiens cDNA: FLJ22816 fis, clone K | 1.88E−07 | 1.19 |
| 412994 | D32257 | Hs. 75113 | general transcription factor IIIA | 1.32E−04 | 1.19 |
| 439077 | AW297762 | Hs. 255690 | ESTs | 2.12E−03 | 1.19 |
| 437714 | AA766346 | | ESTs | 4.83E−04 | 1.19 |
| 407142 | AA412535 | | gb: zt99b10.s1 Soares_testis_NHT Homo sap | 3.74E−04 | 1.18 |
| 407331 | AI570416 | Hs. 99910 | phosphofructokinase, platelet | 5.67E−03 | 1.18 |
| 447960 | AW954377 | Hs. 26412 | ring finger protein 26 | 9.47E−04 | 1.18 |
| 428829 | R14050 | Hs. 194051 | Homo sapiens mRNA; cDNA DKFZp566B213 (fr | 1.47E−05 | 1.18 |
| 426888 | H41090 | Hs. 97373 | ESTs | 6.98E−06 | 1.18 |
| 418539 | AA224753 | Hs. 159500 | ESTs | 1.06E−05 | 1.18 |
| 435037 | AA355113 | Hs. 35380 | x 001 protein | 1.26E−07 | 1.18 |
| 416987 | D86957 | Hs. 80712 | KIAA0202 protein | 8.14E−05 | 1.18 |
| 428828 | AL117508 | Hs. 194035 | KIAA0737 gene product | 2.66E−06 | 1.18 |
| 427433 | D82070 | Hs. 177972 | chromosome 4 open reading frame 6 | 2.70E−05 | 1.18 |
| 434707 | AI378570 | Hs. 116397 | ESTs | 5.75E−04 | 1.18 |
| 445266 | BE222118 | Hs. 12479 | associated molecule with the SH3 domain | 3.36E−05 | 1.18 |
| 427792 | M63928 | Hs. 180841 | tumor necrosis factor receptor superfami | 4.60E−03 | 1.18 |
| 403056 | | | eukaryotic translation elongation factor | 5.76E−04 | 1.18 |
| 415080 | X81789 | Hs. 77897 | splicing factor 3a, subunit 3, 60 kD | 4.92E−04 | 1.18 |
| 418473 | AA243335 | Hs. 309943 | nuclear body protein Sp140 | 2.69E−03 | 1.18 |
| 419270 | NM_005232 | Hs. 89839 | EphA1 | 2.89E−03 | 1.18 |
| 419856 | AK001401 | Hs. 93391 | hypothetical protein FLJ10539 | 5.31E−04 | 1.18 |
| 446207 | AW968535 | Hs. 14328 | hypothetical protein FLJ20071 | 1.49E−05 | 1.18 |
| 416999 | AW195747 | Hs. 21122 | hypothetical protein FLJ11830 similar to | 9.52E−06 | 1.18 |
| 415013 | R35274 | Hs. 24951 | ESTs | 1.63E−04 | 1.18 |
| 426143 | BE379836 | | proteasome (prosome, macropain) subunit, | 3.16E−07 | 1.18 |
| 439815 | AA206079 | Hs. 6693 | hypothetical protein FLJ20420 | 4.52E−07 | 1.18 |
| 412723 | AA648459 | Hs. 335951 | hypothetical protein AF301222 | 2.94E−04 | 1.18 |
| 420882 | AA830074 | Hs. 291915 | ESTs | 5.89E−03 | 1.18 |
| 434198 | AF119849 | Hs. 283028 | hypothetical protein PRO1598 | 2.28E−04 | 1.18 |
| 414602 | AW630088 | Hs. 76550 | Homo sapiens mRNA; cDNA DKFZp564B1264 (f | 5.33E−06 | 1.18 |
| 425729 | L22647 | Hs. 159360 | prostaglandin E receptor 1 (subtype EP1) | 5.73E−03 | 1.18 |
| 420123 | W24330 | Hs. 5301 | hypothetical protein FLJ14936 | 5.77E−05 | 1.18 |
| 456495 | NM_003403 | Hs. 97496 | YY1 transcription factor | 1.59E−05 | 1.18 |
| 440350 | AA883475 | Hs. 257103 | ESTs | 5.34E−03 | 1.18 |
| 437374 | AL359571 | Hs. 44054 | ninein (GSK3B interacting protein) | 1.17E−06 | 1.17 |
| 424006 | AF054815 | Hs. 137548 | CD84 antigen (leukocyte antigen) | 1.93E−04 | 1.17 |
| 444372 | AW377983 | Hs. 298140 | Homo sapiens cDNA: FLJ22502 fis, clone H | 2.17E−07 | 1.17 |
| 426523 | S68616 | Hs. 170222 | solute carrier family 9 (sodium/hydrogen | 1.42E−05 | 1.17 |
| 410388 | AA831460 | | KIAA0758 protein | 2.88E−03 | 1.17 |
| 421660 | R17959 | Hs. 106529 | CGI-65 protein | 8.90E−03 | 1.17 |
| 434759 | BE279230 | Hs. 4113 | S-adenosylhomocysteine hydrolase like 1 | 9.02E−04 | 1.17 |
| 459122 | AI002968 | Hs. 235402 | ESTs, Weakly similar to T26525 hypotheti | 1.59E−03 | 1.17 |
| 450850 | AA648886 | Hs. 151999 | ESTs | 5.32E−06 | 1.17 |
| 416326 | AF186780 | Hs. 79219 | RalGDS-like gene; KIAA0959 protein | 9.26E−03 | 1.17 |
| 450038 | AA005159 | Hs. 188489 | ESTs | 3.51E−03 | 1.17 |
| 447315 | AI613123 | Hs. 342148 | ESTs | 1.28E−05 | 1.17 |
| 426773 | NM_015556 | Hs. 172180 | KIAA0440 protein | 9.85E−03 | 1.17 |
| 443035 | Z45822 | Hs. 8906 | Homo sapiens clone 24889 mRNA sequence | 3.24E−06 | 1.17 |
| 420705 | A8032251 | Hs. 99872 | fetal Alzheimer antigen | 9.87E−04 | 1.17 |
| 431661 | AB037830 | Hs. 267150 | KIAA1409 protein | 7.91E−06 | 1.17 |
| 435314 | AA677891 | Hs. 272138 | ESTs, Weakly similar to ALU1_JHUMAN ALU S | 9.44E−04 | 1.17 |
| 439798 | AA203345 | Hs. 102178 | syntaxin 16 | 6.96E−04 | 1.17 |
| 430629 | AA482392 | Hs. 275184 | ESTs, Weakly similar to ATS1_HUMAN ADAM- | 1.78E−03 | 1.17 |
| 450612 | AL359946 | Hs. 14779 | acetyl-CoA synthetase | 2.31E−04 | 1.17 |
| 416771 | AA868956 | Hs. 13794 | sorting nexin 5 | 2.18E−03 | 1.17 |
| 426244 | AI064808 | Hs. 168289 | succinate dehydrogenase complex, subunit | 1.87E−05 | 1.17 |
| 451338 | AW612322 | Hs. 19131 | transcription factor Dp-2 (E2F dimerizat | 3.41E−05 | 1.17 |
| 443209 | AI040125 | Hs. 150521 | ESTs | 1.48E−03 | 1.17 |
| 458455 | AV648310 | Hs. 213488 | ESTs | 2.67E−03 | 1.17 |
| 446975 | BE246446 | Hs. 16695 | ubiquitin-activating enzyme E1-like | 3.39E−07 | 1.17 |
| 418796 | AA228351 | Hs. 34060 | ESTs | 1.16E−04 | 1.17 |
| 453209 | AW887701 | Hs. 32356 | hypothetical protein FLJ20628 | 1.00E−04 | 1.17 |
| 416240 | NM_001981 | Hs. 79095 | epidermal growth factor receptor pathway | 1.15E−06 | 1.17 |
| 449773 | R76294 | Hs. 302383 | ESTs | 7.90E−03 | 1.17 |
| 435918 | AF263538 | Hs. 86232 | growth differentiation factor 3 | 2.72E−03 | 1.17 |
| 448694 | AA478756 | Hs. 194477 | E3 ubiquitin ligase SMURF2 | 1.00E−05 | 1.17 |
| 445866 | H20899 | Hs. 13399 | Homo sapiens clone 25032 mRNA sequence | 1.92E−05 | 1.17 |
| 411122 | F00809 | Hs. 172004 | coactivator-associated arginine methyltr | 4.93E−04 | 1.17 |
| 426307 | F24978 | Hs. 297962 | ESTs | 1.19E−05 | 1.17 |
| 442107 | AI692286 | | ESTs | 7.80E−03 | 1.17 |
| 416133 | NM_001683 | Hs. 89512 | ATPase, Ca transporting, plasma membrane | 7.02E−04 | 1.17 |
| 445784 | AI253155 | Hs. 146065 | ESTs | 1.22E−04 | 1.17 |
| 427750 | AA693991 | Hs. 224737 | ESTs | 1.79E−03 | 1.16 |
| 406341 | | | C5000881*: gi\|2661590\|emb\|CAA15709.1\|(AL | 5.21E−05 | 1.16 |
| 430006 | BE144167 | Hs. 321056 | hypothetical protein similar to RNA-bind | 2.21E−07 | 1.16 |
| 407821 | AA346172 | Hs. 195614 | ESTs | 2.93E−04 | 1.16 |
| 452299 | AW206330 | | hypothetical protein FLJ10901 | 2.86E−03 | 1.16 |
| 457701 | AW855466 | Hs. 271866 | ESTs, Weakly similar to ALU 1_HUMAN ALU S | 4.53E−05 | 1.16 |
| 446106 | AA377165 | Hs. 44833 | ESTs | 2.32E−05 | 1.16 |
| 415382 | AI743539 | Hs. 72465 | ESTs, Weakly similar to non-lens beta ga | 1.22E−03 | 1.16 |
| 426261 | AW242243 | Hs. 168670 | peroxisomal farnesylated protein | 1.89E−05 | 1.16 |
| 440248 | AA876138 | | ESTs | 8.70E−04 | 1.16 |
| 452600 | AI910842 | Hs. 103381 | ESTs | 2.27E−04 | 1.16 |
| 433023 | AW864793 | | thrombospondin 1 | 6.72E−06 | 1.16 |
| 404694 | | | Target Exon | 6.77E−03 | 1.16 |
| 402668 | | | C1003810: gi\|3766132\|gb\|AAC64377.1\| (AC00 | 1.80E−03 | 1.16 |
| 409141 | AW341059 | Hs. 250664 | EST | 2.66E−03 | 1.16 |
| 418757 | AI864193 | Hs. 169728 | hypothetical protein FLJ13150 | 1.50E−03 | 1.16 |
| 431828 | AA572994 | Hs. 325489 | ESTs | 3.69E−06 | 1.16 |
| 410086 | AI268405 | Hs. 13467 | hypothetical protein MGC3251 | 6.24E−06 | 1.16 |
| 435515 | N40080 | Hs. 6879 | DC13 protein | 4.50E−04 | 1.16 |
| 458965 | AA010319 | Hs. 60389 | ESTs | 1.59E−04 | 1.16 |
| 442127 | AW276851 | Hs. 8107 | Homo sapiens mRNA; cDNA DKFZp586B0918 (f | 7.48E−05 | 1.16 |
| 408127 | AW957168 | Hs. 289064 | hypothetical protein FLJ22251 | 1.95E−07 | 1.16 |
| 446259 | AA425204 | Hs. 334721 | hypothetical protein FLJ13391 | 1.62E−03 | 1.16 |
| 405296 | | | C7000453: gi\|7301410\|gb\|AAF56536.1\| (AE00 | 4.75E−04 | 1.16 |
| 410184 | AW503667 | Hs. 59545 | ring finger protein 15 | 3.38E−06 | 1.16 |
| 445893 | AI610702 | Hs. 202613 | ESTs, Weakly similar to TRHY_HUMAN TRICH | 6.11E−08 | 1.16 |
| 432599 | R99849 | Hs. 269434 | Homo sapiens cDNA FLJ12206 fis, clone MA | 5.84E−05 | 1.16 |
| 417601 | NM_014735 | Hs. 82292 | KIAA0215 gene product | 1.84E−05 | 1.16 |
| 443804 | AL135352 | Hs. 255883 | ESTs, Weakly similar to I38022 hypotheti | 1.76E−04 | 1.16 |
| 422302 | AF018253 | Hs. 114676 | tumor necrosis factor receptor superfami | 3.44E−04 | 1.16 |
| 416215 | BE397505 | Hs. 79081 | protein phospbatase 1, catalytic subunit | 4.93E−08 | 1.16 |
| 433037 | NM_014158 | Hs. 279938 | HSPC067 protein | 1.44E−06 | 1.16 |
| 449971 | AA807346 | Hs. 288581 | Homo sapiens cDNA FLJ14296 fis, clone PL | 4.28E−06 | 1.16 |
| 447313 | U92981 | Hs. 18081 | Homo sapiens clone DT1P1B6 mRNA, CAG rep | 1.16E−03 | 1.16 |
| 430661 | AC005551 | Hs. 130714 | Homo sapiens HSPC323 mRNA, partial cds | 6.11E−04 | 1.16 |
| 445817 | NM_003642 | Hs. 13340 | histone acetyltransferase 1 | 3.99E−04 | 1.16 |
| 430665 | BE350122 | Hs. 157367 | ESTs, Weakly similar to I78885 serine/th | 8.58E−06 | 1.16 |
| 428019 | W85913 | Hs. 30827 | insulin receptor substrate 3-like | 8.44E−03 | 1.16 |
| 440351 | AF030933 | Hs. 7179 | RAD1 (S. pombe) homolog | 3.92E−05 | 1.15 |
| 426455 | AK002196 | Hs. 264076 | Homo sapiens cDNA FLJ11334 fis, clone PL | 1.58E−03 | 1.15 |
| 448905 | BE242730 | Hs. 79093 | EBNA-2 co-activator (100 kD) | 3.95E−03 | 1.15 |
| 429966 | BE081342 | Hs. 283037 | HSPC039 protein | 2.93E−04 | 1.15 |
| 450353 | AI244661 | Hs. 103296 | ESTs, Weakly similar to S65657 alpha-1C- | 3.41E−04 | 1.15 |
| 432650 | D00860 | Hs. 56 | phosphoribosyl pyrophosphale synthetase | 4.23E−07 | 1.15 |
| 453085 | AW954243 | | KIAA0251 protein | 1.99E−03 | 1.15 |
| 407832 | AW976516 | Hs. 283707 | Homo sapiens cDNA: FLJ21354 fis, clone C | 7.53E−04 | 1.15 |
| 442159 | AW163390 | Hs. 278554 | heterochromatin-like protein 1 | 1.35E−05 | 1.15 |
| 444635 | Al184268 | Hs. 339665 | ESTs | 2.17E−03 | 1.15 |
| 432541 | AW131368 | | ESTs | 9.15E−04 | 1.15 |
| 414134 | X60188 | Hs. 861 | mitogen-activated protein kinase 3 | 4.95E−04 | 1.15 |
| 437644 | AA748575 | Hs. 136748 | lectin-like NK cell receptor | 2.69E−06 | 1.15 |
| 409193 | AA131483 | | gb: zo08e05.r1 Stratagene neuroepithelium | 4.60E−03 | 1.15 |
| 422451 | AA310753 | Hs. 42491 | ESTs, Weakly similar to S65657 alpha-1C- | 2.28E−04 | 1.15 |
| 402729 | | | Target Exon | 7.41E−05 | 1.15 |
| 447505 | AL049266 | Hs. 18724 | Homo sapiens mRNA; cDNA DKFZp564F093 (fr | 1.57E−04 | 1.15 |
| 458140 | BE268806 | | gb: 601139917F1 NIH_MGC_8 Homo sapiens cD | 2.74E−05 | 1.15 |
| 424670 | W61215 | Hs. 116651 | epithelial V-like antigen 1 | 2.25E−06 | 1.15 |
| 405122 | | | NM_006798*: Homo sapiens UDP glycosyltran | 6.77E−06 | 1.15 |
| 416022 | R44729 | Hs. 21286 | ESTs | 6.32E−04 | 1.15 |
| 421772 | Z24958 | Hs. 108139 | zinc finger protein 212 | 2.77E−07 | 1.15 |
| 422965 | H90576 | Hs. 28113 | ESTs | 1.18E−03 | 1.15 |
| 430464 | BE393070 | Hs. 188541 | ESTs | 2.44E−03 | 1.15 |
| 416710 | AI268325 | Hs. 54890 | hypothetical protein FLJ23590 | 2.20E−03 | 1.15 |
| 448366 | AI970841 | Hs. 271269 | ESTs | 2.44E−03 | 1.15 |
| 428464 | BE615473 | Hs. 184447 | sirtuin (silent mating type information | 9.06E−04 | 1.15 |
| 427879 | AA435939 | Hs. 98186 | chromosome 21 open reading frame 22 | 2.46E−04 | 1.15 |
| 417226 | AW505054 | Hs. 4283 | ESTs | 3.24E−04 | 1.15 |
| 447757 | AA071276 | Hs. 19469 | KIAA0859 protein | 7.22E−04 | 1.15 |
| 407575 | W27235 | Hs. 64311 | a disintegrin and metalloproteinase doma | 4.80E−04 | 1.15 |
| 446064 | AV656376 | Hs. 282851 | ESTs, Weakly similar to I38022 hypotheti | 1.02E−03 | 115 |
| 425372 | NM_14663 | Hs. 155983 | KIAA0677 gene product | 4.28E−04 | 1.15 |
| 402737 | | | Target Exon | 4.79E−04 | 1.15 |
| 428107 | AA421443 | Hs. 104835 | ESTs | 2.00E−04 | 1.15 |
| 434667 | D61329 | Hs. 32196 | mitochondrial ribosomal protein L36 | 3.49E−06 | 1.15 |
| 423067 | AA321355 | Hs. 285401 | colony stimulating factor 2 receptor, be | 2.94E−03 | 1.15 |
| 445208 | AI216103 | | ESTs | 1.56E−03 | 1.15 |
| 439581 | AI539852 | Hs. 118738 | KIAA0800 gene product | 6.14E−03 | 1.15 |
| 446012 | AV656098 | Hs. 172382 | betainE-homocysteine methyltransferase 2 | 1.27E−04 | 1.15 |
| 437234 | AI472213 | Hs. 247711 | hypothetical protein FLJ20557 | 6.90E−03 | 1.15 |
| 408203 | AA053137 | Hs. 42390 | nasopharyngeal carcinoma susceptibility | 6.04E−05 | 1.15 |
| 456352 | AA233094 | Hs. 191517 | ESTs | 3.71E−03 | 1.15 |
| 422497 | D29642 | Hs. 1528 | KIAA0053 gene product | 9.72E−05 | 1.15 |
| 414762 | AW068349 | Hs. 77257 | KIAA0068 protein | 9.50E−05 | 1.15 |
| 458799 | AI688547 | | ESTs | 3.89E−03 | 1.15 |
| 434271 | AA897778 | Hs. 201677 | ESTs, Weakly similar to Z257_HUMAN ZINC | 6.94E−03 | 1.15 |
| 410423 | AW402432 | Hs. 63489 | protein tyrosine phosphatase, non-recept | 4.56E−04 | 1.15 |
| 434421 | AI915927 | Hs. 34771 | ESTs | 2.00E−04 | 1.14 |
| 423623 | AB011117 | Hs. 129943 | KIAA0545 protein | 1.39E−09 | 1.14 |
| 404231 | | | Target Exon | 2.75E−03 | 1.14 |
| 423717 | AA330036 | Hs. 152003 | ESTs | 8.79E−03 | 1.14 |
| 453370 | AI470523 | Hs. 139336 | ATP-binding cassette, sub-family C (CFTR | 6.17E−05 | 1.14 |
| 420820 | W26096 | | Homo sapiens, clone IMAGE: 4179482, mRNA | 1.41E−09 | 1.14 |
| 448959 | AI610343 | Hs. 293267 | ESTs | 2.10E−04 | 1.14 |
| 423582 | BE000831 | Hs. 23837 | Homo sapiens cDNA FLJ11812 fis, clone HE | 6.93E−03 | 1.14 |
| 428310 | AF100749 | Hs. 183655 | sec22 homolog | 5.55E−06 | 1.14 |
| 405287 | | | Target Exon | 7.91E−04 | 1.14 |
| 426532 | AA486725 | | hypothetical protein FLJ20489 | 7.45E−04 | 1.14 |
| 423494 | AW504365 | Hs. 24143 | Wiskott-Aldrich syndrome protein interac | 1.50E−03 | 1.14 |
| 446161 | AA628206 | Hs. 14125 | p53 regulated PA26 nuclear protein | 1.05E−06 | 1.14 |
| 433481 | AL040235 | Hs. 3346 | hypothetical protein FLJ11280 | 3.23E−03 | 1.14 |
| 434072 | H70854 | | Homo sapiens PRO1082 mRNA, complete cds | 7.75E−04 | 1.14 |
| 427691 | AW194426 | Hs. 20726 | ESTs | 3.63E−04 | 1.14 |
| 437825 | AA769123 | Hs. 291947 | ESTs | 9.72E−04 | 1.14 |
| 430255 | AK000703 | Hs. 323822 | Homo sapiens mRNA for KIAA1551 protein, | 3.00E−04 | 1.14 |
| 414872 | U82010 | Hs. 77513 | COX10 (yeast) homolog. cytochrome c oxid | 4.26E−03 | 1.14 |
| 439741 | BE379646 | Hs. 6904 | Homo sapiens mRNA full length insert cDN | 7.07E−06 | 1.14 |
| 417025 | AA262170 | Hs. 80917 | adaptor-related protein complex 3, sigma | 4.41E−05 | 1.14 |
| 418917 | X02994 | Hs. 1217 | adenosine deaminase | 2.96E−03 | 1.14 |
| 426367 | AW611704 | Hs. 48578 | ESTs | 7.75E−03 | 1.14 |
| 423855 | AA331761 | Hs. 254859 | ESTs | 5.33E−05 | 1.14 |
| 450832 | AW970602 | Hs. 105421 | ESTs | 4.74E−04 | 1.14 |
| 449028 | BE616023 | Hs. 25298 | KIAA1813 protein | 5.52E−04 | 1.14 |
| 426384 | AI472078 | Hs. 303662 | hypothetical protein FLJ13189 (FLJ13189) | 1.75E−03 | 1.14 |
| 429652 | AA766810 | Hs. 259290 | ESTs | 6.17E−03 | 1.14 |
| 415205 | H71616 | Hs. 135233 | ESTs | 5.85E−06 | 1.14 |
| 436860 | H12751 | Hs. 5327 | PRO1914 protein | 1.93E−06 | 1.14 |
| 427973 | AW851123 | Hs. 98286 | gb: IL3-CT0220-150200-070-H02 CT0220 Homo | 1.93E−03 | 1.14 , |
| 426940 | AA393537 | Hs. 98347 | ESTs, Weakly similar to JC5308 testis-sp | 2.52E−03 | 1.14 |
| 455588 | AI129903 | Hs. 74669 | vesicle-associated membrane protein 5 (m | 4.99E−06 | 1.14 |
| 422532 | AL008726 | Hs. 118126 | protective protein for beta-galactosidas | 1.22E−03 | 1.14 |
| 431316 | AA502663 | Hs. 145037 | ESTs | 1.70E−04 | 1.14 |
| 408329 | AF155510 | Hs. 44227 | heparanase | 7.40E−03 | 1.14 |
| 457415 | AK000010 | Hs. 258798 | hypothetical protein FLJ20003 | 9.74E−04 | 1.14 |
| 409509 | AL036923 | Hs. 322710 | ESTs | 8.97E−03 | 1.14 |
| 414713 | BE465243 | Hs. 12664 | ESTs | 8.42E−03 | 1.13 |
| 422567 | AF111178 | Hs. 118407 | glypican 6 | 4.15E−08 | 1.13 |
| 457346 | AI734178 | Hs. 93379 | ESTs, Highly similar to 1207289A reverse | 7.22E−03 | 1.13 |
| 449559 | AW614016 | Hs. 282131 | ESTs | 8.76E−05 | 1.13 |
| 447932 | AA837474 | Hs. 20021 | vesicle-associated membrane protein 1 (s | 3.47E−06 | 1.13 |
| 434981 | AW182577 | Hs. 293077 | ESTs | 1.26E−03 | 1.13 |
| 452186 | M120761 | Hs. 28307 | WW domain binding protein 4 (formin bind | 6.11E−03 | 1.13 |
| 442161 | AI733230 | Hs. 128805 | ESTs | 6.46E−04 | 1.13 |
| 438580 | AA811262 | Hs. 299202 | ESTs | 6.12E−04 | 1.13 |
| 424941 | AA128376 | Hs. 153884 | ATP binding protein associated with cell | 7.86E−06 | 1.13 |
| 424867 | AI024860 | Hs. 153591 | Not56 (D. melanogaster)-like protein | 1.15E−04 | 1.13 |
| 455063 | AW854160 | | gb: RC3-CT0254-060400-029-f09 CT0254 Homo | 4.21E−04 | 1.13 |
| 431874 | AW610031 | Hs. 323914 | translocase of inner milochondrial membr | 6.87E−04 | 1.13 |
| 405695 | | | Target Exon | 5.77E−03 | 1.13 |
| 424065 | NM_016388 | Hs. 138701 | T-cell receptor interacting molecule | 9.24E−05 | 1.13 |
| 441192 | AA526626 | Hs. 7736 | mitochondrial ribosomal protein L27 | 3.59E−05 | 1.13 |
| 450316 | W84446 | Hs. 226434 | hypothetical protein M6C4643 | 5.68E−05 | 1.13 |
| 456859 | X92521 | Hs. 154057 | matrix metalloproteinase 19 | 3.33E−03 | 1.13 |
| 421639 | NM_012082 | Hs. 106309 | Friend of GATA2 | 5.03E−03 | 1.13 |
| 427844 | AV658308 | Hs. 2210 | thyroid hormone receptor interactor 3 | 4.55E−05 | 1.13 |
| 412019 | AA485890 | Hs. 69330 | Homo sapiens cDNA FLJ13835 fis. clone TH | 4.85E−06 | 1.13 |
| 441892 | AB028981 | Hs. 8021 | KIAA1058 protein | 7.83E−06 | 1.13 |
| 416033 | NM_012201 | Hs. 78979 | Golgi apparatus protein 1 | 1.08E−04 | 1.13 |
| 450455 | AL117424 | Hs. 25035 | chloride intracellular channel 4 | 3.45E−05 | 1.13 |
| 406658 | AI920965 | Hs. 77961 | major histocompatibility complex, class | 3.18E−05 | 1.13 |
| 406827 | AA971409 | | gb: op92c04.s1 NCI_CGAP_Lu5 Homo sapiens | 2.85E−05 | 1.13 |
| 457065 | AI476318 | Hs. 192480 | ESTs | 4.47E−03 | 1.13 |
| 425538 | BE270918 | Hs. 164026 | Homo sapiens, clone IMAGE: 3534875, mRNA, | 1.63E−03 | 1.13 |
| 416852 | AF283776 | Hs. 80285 | Homo sapiens mRNA; cDNA DKFZp586C1723 (f | 4.61E−04 | 1.13 |
| 422929 | AA356694 | Hs. 94011 | ESTs, Weakly similar to MGB4_HUMAN MELAN | 1.74E−04 | 1.13 |
| 435274 | AA887547 | Hs. 150905 | ESTs | 7.03E−06 | 1.13 |
| 415825 | Y18024 | Hs. 78877 | inositol 1,4,5-trisphosphate 3-kinase B | 6.01E−04 | 1.13 |
| 416957 | U86749 | Hs. 80598 | transcription elongation factor A (Sll), | 4.33E−06 | 1.13 |
| 420721 | AA927802 | Hs. 159471 | ZAP3 protein | 3.70E−03 | 1.13 |
| 421313 | NM_014923 | Hs. 103329 | KIAA0970 protein | 1.14E−04 | 1.13 |
| 420885 | NM_005652 | Hs. 100030 | telomeric repeat binding factor 2 | 1.65E−05 | 1.13 |
| 437156 | AI916600 | Hs. 121194 | Homo sapiens cDNA: FLJ21569 fis, clone C | 1.03E−03 | 1.13 |
| 436614 | AW104388 | Hs. 149091 | ESTs | 6.83E−03 | 1.13 |
| 451591 | AA886446 | Hs. 23590 | solute carrier family 16 (monocarboxylic | 5.25E−04 | 1.13 |
| 445977 | BE550048 | Hs. 145735 | ESTs | 7.77E−05 | 1.13 |
| 401112 | | | NM_024997*: Homo sapiens hypothetical pro | 2.93E−03 | 1.13 |
| 418096 | AA282660 | Hs. 193520 | ESTs | 3.07E−03 | 1.13 |
| 410247 | AF181721 | Hs. 61345 | RU2S | 2.71E−03 | 1.13 |
| 407754 | AA527348 | Hs. 288967 | Homo sapiens cDNA FLJ14105 fis, clone MA | 2.18E−03 | 1.13 |
| 403671 | | | C4001270*: gi\|7509005\|pir\|\|T26190 hypothe | 2.04E−04 | 1.13 |
| 448294 | AI479918 | Hs. 192859 | ESTs | 5.40E−04 | 1.13 |
| 414726 | BE466863 | Hs. 280099 | ESTs | 1.35E−04 | 1.13 |
| 422489 | AA311216 | Hs. 183953 | ESTs | 3.48E−03 | 1.13 |
| 429595 | AI910584 | Hs. 127639 | ESTs | 9.52E−04 | 1.13 |
| 419854 | AW664873 | Hs. 87836 | Homo sapiens PAC clone RP5-1087M19 from | 1.17E−05 | 1.12 |
| 418522 | AA605038 | Hs. 7149 | Homo sapiens cDNA: FLJ21950 (is, clone H | 2.05E−05 | 1.12 |
| 452717 | AW160399 | Hs. 30376 | hypothetical protein | 9.03E−04 | 1.12 |
| 445495 | BE622641 | Hs. 38489 | ESTs, Weakly similar to I38022 hypotheti | 6.31E−05 | 1.12 |
| 438724 | AW612553 | Hs. 114670 | Human DNA sequence from clone RP11-16L21 | 7.89E−08 | 1.12 |
| 426110 | NM_002913 | Hs. 166563 | replication factor C (activator 1) 1 (14 | 5.22E−09 | 1.12 |
| 416411 | AA180188 | Hs. 10706 | ADP-ribosylation factor-like 4 | 1.05E−04 | 1.12 |
| 422614 | AI908006 | Hs. 295362 | Homo sapiens cDNA FLJ14459 fis, clone HE | 5.79E−04 | 1.12 |
| 438650 | AA843728 | Hs. 123533 | ESTs | 5.65E−03 | 1.12 |
| 437175 | AW968078 | Hs. 87773 | protein kinase, cAMP-dependent, catalyti | 2.02E−O6 | 1.12 |
| 445278 | BE391904 | Hs. 12482 | glyceronephosphate O-acyltransferase | 1.81E−03 | 1.12 |
| 424577 | AI346124 | Hs. 28813 | ESTs, Weakly similar to A23772 UNE-1 hy | 1.80E−03 | 1.12 |
| 410575 | BE207480 | Hs. 6994 | Homo sapiens cDNA: FLJ22044 fis, clone H | 2.56E−06 | 1.12 |
| 424779 | AL046851 | Hs. 153053 | CD37 antigen | 7.40E−03 | 1.12 |
| 452057 | AW952005 | Hs. 14928 | hypothetical protein FLJ12903 | 2.36E−04 | 1.12 |
| 427008 | Z45258 | Hs. 286013 | short coiled-coil protein | 2.52E−06 | 1.12 |
| 417944 | AU077196 | Hs. 82985 | collagen, type V, alpha 2 | 6.04E−05 | 1.12 |
| 443260 | AW896265 | Hs. 127310 | ESTs, Weakly similar to 1805352A splicin | 3.66E−05 | 1.12 |
| 420152 | AW968080 | Hs. 171553 | Homo sapiens clone 24630 mRNA sequence | 2.74E−05 | 1.12 |
| 430050 | AA430993 | Hs. 227913 | APIS-like 1 | 6.41E−04 | 1.12 |
| 425302 | U79115 | Hs. 155566 | CASP2 and RIPK1 domain containing adapto | 9.39E−05 | 1.12 |
| 411988 | AA455459 | Hs. 164480 | ESTs, Weakly similar to T50609 hypotheti | 3.48E−05 | 1.12 |
| 406657 | AI678644 | Hs. 277477 | major histocompatibility complex, class | 2.78E−05 | 1.12 |
| 421940 | AA311445 | Hs. 9052 | Homo sapiens cDNA: FLJ22042 fis, clone H | 1.17E−05 | 1.12 |
| 458812 | W57902 | Hs. 90744 | proteasome (prosome, macropain) 26S subu | 2.72E−06 | 1.12 |
| 409008 | AA059392 | Hs. 66791 | ESTs | 2.07E−04 | 1.12 |
| 410700 | AA352335 | Hs. 65641 | hypothetical protein FLJ 20073 | 4.95E−04 | 1.12 |
| 458229 | AI929602 | Hs. 177 | phosphatidylinositol glycan, class H | 6.18E−03 | 1.12 |
| 400886 | | | Target Exon | 4.47E−04 | 1.12 |
| 422993 | AF154415 | Hs. 122843 | CASP8 associated protein 2 | 1.49E−03 | 1.12 |
| 452501 | AB037791 | Hs. 29716 | hypothetical protein FLJ 10980 | 1.47E−03 | 1.12 |
| 443202 | AW843882 | Hs. 19669 | ESTs, Weakly similar to T17346 hypotheti | 8.03E−04 | 1.12 |
| 402474 | | | NM_004079: Homo sapiens cathepsin S (CTSS | 7.68E−07 | 1.12 |
| 440993 | A1274880 | Hs146339 | ESTs | 6.87E−03 | 1.12 |
| 400157 | | | Eos Control | 5.48E−05 | 1.12 |
| 449209 | BE616830 | Hs. 294145 | ESTs | 5.38E−07 | 1.12 |
| 446771 | AA128965 | Hs. 60679 | TATA box binding protein (TBP)-associate | 6.52E−07 | 1.12 |
| 433533 | D53304 | Hs. 9028 | fusion, derived from t(12;16) malignant | 1.09E−04 | 1.12 |
| 430799 | C19035 | Hs. 164259 | ESTs | 6.28E−04 | 1.12 |
| 453870 | AW385001 | Hs. 8042 | Homo sapiens cDNA: FLJ23173 fis, clone L | 1.66E−03 | 1.12 |
| 421748 | NM_014718 | Hs. 107809 | KIAA0726 gene product | 8.58E−05 | 1.11 |
| 414181 | AK000476 | Hs. 75798 | hypothetical protein | 5.55E−06 | 1.11 |
| 409327 | L41162 | Hs. 53563 | collagen, type IX, alpha 3 | 2.49E−05 | 1.11 |
| 407804 | AF228603 | Hs. 39957 | pleckstrin 2 (mouse) homolog | 2.09E−03 | 1.11 |
| 434821 | AA159111 | Hs. 284281 | Human putative ribosomal protein S1 mRNA | 2.44E−07 | 1.11 |
| 411887 | AW182924 | Hs. 128790 | ESTs | 4.06E−03 | 1.11 |
| 414515 | AA947457 | Hs. 135560 | ESTs, Weakly similar to T43458 hypotheti | 8.13E−03 | 1.11 |
| 440706 | AA927562 | Hs. 148234 | ESTs | 2.22E−03 | 1.11 |
| 424928 | AW753193 | Hs. 188361 | Homo sapiens cDNA FLJ12807 fis, clone NT | 1.01E−03 | 1.11 |
| 410342 | R31350 | Hs. 743 | Fc fragment of IgE, high affinity I, rec | 7.13E−03 | 1.11 |
| 449261 | AI637592 | | ESTs | 5.50E−03 | 1.11 |
| 448391 | H71025 | Hs. 21075 | GTF2I repeat domain-containing 1 | 4.49E−06 | 1.11 |
| 419384 | AA490866 | Hs. 39429 | ESTs | 9.16E−04 | 1.11 |
| 414256 | AW410035 | Hs. 75862 | MAD (mothers against decapentaplegic, Dr | 3.56E−04 | 1.11 |
| 451652 | AA018968 | Hs. 133536 | ESTs | 2.21E−05 | 1.11 |
| 430235 | BE268048 | Hs. 236494 | RAB10, member RAS oncogene family | 2.93E−06 | 1.11 |
| 452694 | AK001736 | Hs. 30318 | hypothetical protein FLJ10874 | 9.45E−03 | 1.11 |
| 444377 | AA134369 | Hs. 11042 | hypothetical protein | 5.02E−07 | 1.11 |
| 420309 | AW043637 | Hs. 21766 | ESTs, Weakly similar to ALU5_HUMAN ALU S | 1.91E−04 | 1.11 |
| 436845 | AA732297 | Hs. 113928 | ESTs | 2.01E−03 | 1.11 |
| 443530 | BE563088 | Hs. 9552 | binder of Arl Two | 1.45E−04 | 1.11 |
| 414618 | AI204600 | Hs. 96978 | hypothetical protein MGC10764 | 8.68E−04 | 1.11 |
| 434636 | AA083764 | | hypothetical protein MGC3178 | 4.36E−04 | 1.11 |
| 422001 | AW891476 | Hs. 122764 | BRCA1 associated protein | 2.23E−03 | 1.11 |
| 413186 | AU077141 | | solute carrier family 16 (monocarboxylic | 2.27E−04 | 1.11 |
| 423692 | BE621056 | Hs. 131731 | hypothetical protein FLJ11099 | 1.29E−03 | 1.11 |
| 417890 | R79048 | | gb: yi87gO2.r1 Soares placenta Nb2HP Homo | 3.79E−03 | 1.11 |
| 453823 | AL137967 | | gb: DKFZp761D2315_r1 761 (synonym: hamy2) | 8.96E−03 | 1.11 |
| 431976 | AA719001 | Hs. 291065 | ESTs | 3.97E−06 | 1.11 |
| 421183 | AL135740 | Hs. 102447 | TSC-22-like | 1.31E−04 | 1.11 |
| 417382 | AI818489 | Hs. 139709 | hypothetical protein FLJ12572 | 6.22E−03 | 1.11 |
| 436643 | AA757626 | Hs. 10941 | ESTs, Weakly similar to IPP1_HUMAN PROTE | 6.13E−06 | 1.11 |
| 436763 | AI168278 | | ESTs | 9.15E−03 | 1.11 |
| 422426 | W79117 | Hs. 58559 | ESTs | 7.92E−03 | 1.11 |
| 418018 | AW973655 | Hs. 286184 | hypothetical protein dJ551D2.5 | 8.85E−03 | 1.11 |
| 425145 | BE242802 | Hs. 154797 | KIAA0090 protein | 4.53E−04 | 1.11 |
| 435313 | AI769400 | Hs. 189729 | ESTs | 3.81E−03 | 1.11 |
| 421305 | BE397354 | Hs. 324830 | diptheria toxin resistance protein requi | 5.71E−03 | 1.11 |
| 427939 | T92459 | Hs. 16886 | ESTs | 1.26E−03 | 1.11 |
| 433365 | AF026944 | Hs. 293797 | ESTs | 1.03E−04 | 1.11 |
| 458524 | N63262 | Hs. 135965 | ESTs | 1.76E−03 | 1.11 |
| 419590 | AFO05043 | Hs. 91390 | poly (ADP-ribose) glycohydrolase | 4.96E−06 | 1.11 |
| 418224 | AL036057 | Hs. 83795 | interferon regulatory lactor 2 | 3.93E−05 | 1.10 |
| 429272 | W25140 | Hs. 110667 | ESTs | 3.57E−03 | 1.10 |
| 452586 | AW958479 | Hs. 289043 | spindlin | 9.58E−05 | 1.10 |
| 438363 | AI886351 | Hs. 22353 | hypothetical protein FLJ21952 | 8.36E−06 | 1.10 |
| 437042 | AK000702 | Hs. 5420 | hypothetical protein FLJ20695 | 3.05E−03 | 1.10 |
| 457130 | NM_005651 | Hs. 183671 | tryptophan 2,3-dioxygenase | 9.64E−06 | 1.10 |
| 456651 | AA302420 | Hs. 163622 | ESTs | 1.91E−03 | 1.10 |
| 446002 | AI346468 | | ESTs | 1.53E−04 | 1.10 |
| 441389 | AF134838 | Hs. 7835 | endocytic receptor (macrophage mannose r | 3.16E−03 | 1.10 |
| 442524 | H45879 | Hs. 37288 | nuclear receptor subfamily 1, group D, m | 1.56E−04 | 1.10 |
| 437888 | AA373314 | Hs. 5897 | Homo sapiens mRNA; cDNA DKFZp586P1622 (f | 1.82E−08 | 1.10 |
| 448822 | BE149845 | Hs. 289038 | hypothetical protein MGC4126 | 2.67E−04 | 1.10 |
| 409835 | AW501362 | | gb: UI-HF-BP0p-aiv-a-08-0-Ul.r1 NIH_MGC_5 | 2.67E−04 | 1.10 |
| 420181 | AI380089 | Hs. 158951 | ESTs | 6.82E−04 | 1.10 |
| 457650 | AA649162 | Hs. 236456 | ESTs | 3.31E−09 | 1.10 |
| 409717 | AW452871 | Hs. 56043 | CGI-115 protein | 3.69E−03 | 1.10 |
| 408831 | AF090114 | Hs. 48433 | endocrine regulator | 5.13E−05 | 1.10 |
| 420131 | F08286 | Hs. 95262 | nuclear factor related to kappa B bindin | 2.43E−05 | 1.10 |
| 427468 | AB036829 | Hs. 178347 | SKIP for skeletal muscle and kidney enri | 3.88E−04 | 1.10 |
| 442990 | AA197226 | Hs. 19347 | hypothetical protein MGC11321 | 1.85E−04 | 1.10 |
| 420867 | NM_014183 | Hs. 100002 | HSPC162 protein | 1.64E−08 | 1.10 |
| 450401 | AW959281 | Hs. 8184 | ESTs | 4.53E−04 | 1.10 |
| 453259 | R93125 | Hs. 124187 | ESTs | 4.73E−03 | 1.10 |
| 448384 | R12314 | Hs. 21056 | Homo sapiens cDNA: FLJ21366 fis, clone C | 1.32E−04 | 1.10 |
| 446141 | AW631255 | Hs. 324470 | L-3-hydroxyacyl-Coenzyme A dehydrogenase | 4.46E−04 | 1.10 |
| 428746 | AW503820 | Hs. 192861 | Spi-B transcription factor (Spi-1/PU.1 r | 1.56E−03 | 1.10 |
| 435523 | T62849 | Hs. 11090 | membrane-spanning 4-domains, subfamily A | 3.95E−06 | 1.10 |
| 418816 | T29621 | Hs. 88778 | carbonyl reductase 1 | 2.36E−08 | 1.10 |
| 450222 | U75308 | Hs. 24644 | TATA box binding protein (TBP)-associate | 3.72E−04 | 1.10 |
| 408633 | AW963372 | Hs. 46677 | PRO2000 protein | 8.12E−04 | 1.10 |
| 439898 | AW505514 | Hs. 209561 | KIAA1715 protein | 3.80E−04 | 1.10 |
| 411111 | AW818127 | | gb: CM1-ST0277-061299-059-b07 ST0277 Homo | 5.18E−04 | 1.10 |
| 435010 | N89307 | Hs. 124696 | oxidoreductase UCPA | 2.08E−07 | 1.10 |
| 434039 | L32977 | Hs. 3712 | ubiquinol-cytochrome c reductase, Rieske | 4.10E−04 | 1.10 |
| 417094 | NM_006895 | Hs. 81182 | histamine N-methyltransferase | 4.50E−07 | 1.10 |
| 408655 | AJ243950 | Hs. 46735 | deafness locus associated putative guani | 1.30E−04 | 1.10 |
| 421968 | W22587 | Hs. 110095 | Homo sapiens mRNA; cDNA DKFZp761E0711(f | 3.52E−03 | 1.10 |
| 427172 | AA398844 | Hs. 64595 | ESTs, Weakly similar to AF151831 1 CGI-7 | 2.07E−06 | 1.10 |
| 426059 | BE292842 | Hs. 166120 | interferon regulatory factor 7 | 4.26E−06 | 1.10 |
| 408047 | AW205461 | Hs. 243612 | ESTs | 5.99E−05 | 1.10 |
| 446616 | R65964 | Hs. 334873 | ESTs, Weakly similar to ALU8_HUMAN ALU S | 1.80E−03 | 1.10 |
| 423252 | AB005289 | Hs. 125856 | ATP-binding cassette, sub-family B (MDR/ | 5.53E−03 | 1.09 |
| 437869 | W91976 | Hs. 290834 | ESTs | 2.89E−05 | 1.09 |
| 412577 | Z22968 | Hs. 74076 | CD163 antigen | 8.49E−06 | 1.09 |
| 420664 | AI681270 | Hs. 99824 | BCE-1 protein | 1.33E−06 | 1.09 |
| 432891 | AF161483 | Hs. 279761 | HSPC134 protein | 9.54E−03 | 1.09 |
| 448346 | AW515030 | Hs. 258526 | ESTs | 1.07E−04 | 1.09 |
| 408594 | BE144666 | | gb: CM2-HT0176-041099-017-c02 HT0176 Homo | 4.37E−05 | 1.09 |
| 437730 | AW071087 | Hs. 239176 | insulin-like growth factor 1 receptor | 3.49E−03 | 1.09 |
| 414966 | BE544579 | Hs. 77690 | RAB5B, member RAS oncogene family | 8.08E−03 | 1.09 |
| 422722 | H74219 | Hs. 269772 | ESTs | 1.24E−03 | 1.09 |
| 446392 | AF142419 | Hs. 15020 | homolog of mouse quaking QKI (KH domain | 8.53E−05 | 1.09 |
| 405064 | | | Target Exon | 4.07E−03 | 1.09 |
| 453555 | N23574 | Hs. 123649 | ESTs, Moderately similar to ALU7_HUMAN A | 3.02E−06 | 1.09 |
| 448928 | AI350260 | Hs. 301539 | mitochondrial ribosomal protein L34 | 2.32E−03 | 1.09 |
| 421643 | BE281170 | Hs. 106357 | valosin-containing protein | 8.84E−06 | 1.09 |
| 436749 | AA584890 | Hs. 5302 | leclin, galactoside-binding, soluble, 4 | 9.82E−04 | 1.09 |
| 449223 | AB002348 | Hs. 23263 | KIAA0350 protein | 3.59E−05 | 1.09 |
| 446927 | AW503484 | Hs. 16533 | myosin phosphatase, target subunit 1 | 1.36E−03 | 1.09 |
| 439616 | BE018635 | Hs. 58582 | Homo sapiens cDNA FLJ12789 fis, clone NT | 4.53E−06 | 1.09 |
| 433361 | AW469373 | | ribosomal protein L39 | 6.62E−03 | 1.09 |
| 410781 | AI375672 | Hs. 165028 | ESTs | 4.93E−05 | 1.09 |
| 421535 | AB002359 | Hs. 105478 | phosphoribosylformylglycinamidine syntha | 6.66E−04 | 1.09 |
| 442079 | AW183787 | | ESTs | 2.49E−03 | 1.09 |
| 443274 | BE540891 | | protein kinase, cAMP-dependent, regulato | 3.02E−04 | 1.09 |
| 437070 | AA765471 | Hs. 154494 | ESTs, Weakly similar to 2109260A B cell | 4.49E−03 | 1.09 |
| 424690 | BE538356 | Hs. 151777 | eukaryotic translation initiation factor | 1.30E−05 | 1.09 |
| 418510 | R94322 | Hs. 271642 | ESTs, Weakly similar to I38022 hypotheti | 2.87E−03 | 1.09 |
| 422773 | AB028962 | Hs. 301552 | KIAA1039 protein | 1.54E−03 | 1.09 |
| 436907 | AA737171 | Hs. 131809 | ESTs | 1.38E−05 | 1.09 |
| 420087 | AI653368 | Hs. 81889 | ESTs | 2.56E−03 | 1.09 |
| 426407 | BE272112 | Hs. 48563 | ESTs, Moderately similar to ALU2_HUMAN A | 3.11E−04 | 1.09 |
| 425917 | W28517 | Hs. 117167 | Homo sapiens cONA: FLJ23067 fis, clone L | 5.77E−08 | 1.09 |
| 417973 | NM_004490 | Hs. 83070 | growth factor receptor-bound protein 14 | 2.92E−03 | 1.09 |
| 444026 | AA205759 | Hs. 10119 | hypothetical protein FLJ14957 | 1.40E−06 | 1.09 |
| 413840 | AI301558 | | RNA binding motif protein, X chromosome | 1.30E−04 | 1.09 |
| 451612 | AI928962 | Hs. 26761 | DKFZP586L0724 protein | 3.91E−05 | 1.09 |
| 414839 | X63692 | Hs. 77462 | DNA (cytosine-5-)-methyltransferase 1 | 1.24E−03 | 1.09 |
| 438827 | AI288188 | Hs. 32124 | ESTs, Moderately similar to ALU1 _HUMAN A | 9.62E−06 | 1.09 |
| 438897 | R52285 | Hs. 289018 | Homo sapiens cDNA: FLJ22918 fis, clone K | 2.03E−03 | 1.08 |
| 427547 | BE047653 | Hs. 119183 | ESTs, Weakly similar to ZN91_HUMAN ZINC | 4.93E−04 | 1.08 |
| 439205 | AF087990 | Hs. 42758 | Homo sapiens, clone IMAGE: 3354845, mRNA, | 4.77E−03 | 1.08 |
| 427095 | AA316080 | Hs. 173554 | ubiquinol-cytochrome c reductase core pr | 4.13E−06 | 1.08 |
| 425154 | NM_001851 | Hs. 154850 | collagen, type IX, alpha 1 | 1.08E−03 | 1.08 |
| 426466 | BE619053 | Hs. 170001 | eukaryotic translation initiation factor | 2.34E−04 | 1.08 |
| 413580 | T64632 | Hs. 202974 | ESTs, Weakly similar to B57785 zinc fing | 1.99E−03 | 1.08 |
| 443084 | AI827639 | Hs. 125539 | ESTs | 1.18E−03 | 1.08 |
| 434724 | AA644555 | Hs. 136568 | ESTs | 3.11E−03 | 1.08 |
| 429292 | AW161453 | Hs. 198767 | COP9 (constitutive photomorphogenic, Ara | 2.29E−09 | 1.08 |
| 423120 | AW160551 | Hs. 124021 | soggy-1 gene | 1.53E−03 | 1.08 |
| 433350 | BE563152 | Hs. 10362 | Homo sapiens cDNA: FLJ20944 fis, clone A | 1.35E−06 | 1.08 |
| 421759 | AA027968 | Hs. 107979 | small membrane protein 1 | 4.31E−05 | 1.08 |
| 434645 | AF255303 | Hs. 112227 | membrane-associated nucleic acid binding | 1.47E−08 | 1.08 |
| 435632 | AF220049 | Hs. 43549 | uncharacterized hematopoietic stem/proge | 3.86E−04 | 1.08 |
| 410196 | AI936442 | Hs. 59838 | hypothetical protein FLJ10808 | 3.40E−05 | 1.08 |
| 430010 | AI434760 | Hs. 279949 | KIAA1007 protein | 2.32E−03 | 1.08 |
| 402007 | | | C18000503: gi\|8922165\|ref\|NP_060080.1\| h | 3.30E−03 | 1.08 |
| 423303 | AW593818 | Hs. 186421 | ESTs | 6.72E−03 | 1.08 |
| 445529 | H14421 | Hs. 180513 | ATP-binding cassette, sub-family A (ABC1 | 2.31E−04 | 1.08 |
| 414038 | BE242722 | Hs. 180040 | hypothetical protein FLJ22439 | 3.84E−05 | 1.08 |
| 456640 | AW207000 | Hs. 126857 | Homo sapiens cDNA FLJ12936 fis, clone NT | 2.91E−07 | 1.08 |
| 450649 | NM_001429 | Hs. 25272 | E1A binding protein p300 | 3.10E−03 | 1.08 |
| 422461 | NM_003417 | Hs. 117077 | zinc finger protein 264 | 4.28E−04 | 1.08 |
| 411850 | AK002033 | Hs. 72782 | hypothetical protein FLJ11171 | 6.36E−04 | 1.08 |
| 433847 | AA610266 | Hs. 3631 | ESTs | 5.50E−04 | 1.08 |
| 431049 | AA846576 | Hs. 103267 | hypothetical protein FLJ22548 similar to | 1.17E−05 | 1.08 |
| 400967 | | | Target Exon | 2.95E−03 | 1.08 |
| 415987 | R41275 | Hs. 346886 | gb: yf85c07.s1 Soares infant brain 1NIB H | 2.19E−04 | 1.08 |
| 414427 | L19711 | Hs. 76111 | dystroglycan 1 (dystrophin-associated gl | 5.18E−07 | 1.08 |
| 416039 | AA376989 | Hs. 78989 | alcohol dehydrogenase 5 (class III), chi | 2.67E−05 | 1.08 |
| 438951 | U51336 | Hs. 6453 | inositol 1,3,4-triphosphate 5/6 kinase | 7.07E−04 | 1.08 |
| 427979 | BE379776 | Hs. 181309 | proteasome (prosome, macropain) subunit. | 1.02E−05 | 1.08 |
| 419852 | AW503756 | Hs. 286184 | hypothetical protein dJ551D2.5 | 8.58E−06 | 1.08 |
| 456115 | F01082 | Hs. 172004 | titin | 3.41E−03 | 1.08 |
| 416396 | R91986 | Hs. 209508 | ESTs, Moderately similar to ALU1_HUMAN A | 7.17E−05 | 1.08 |
| 419080 | AW150835 | Hs. 18878 | hypothetical protein FLJ21620 | 3.78E−05 | 1.08 |
| 421013 | M62397 | Hs. 1345 | mutated in colorectal cancers | 9.61E−04 | 1.08 |
| 425465 | L18964 | Hs. 1904 | protein kinase C, iota | 1.12E−04 | 1.08 |
| 433198 | AA992841 | HS27263 | KIAA1458 protein | 4.26E−03 | 1.08 |
| 456792 | BE502835 | Hs. 15463 | Homo sapiens, clone IMAGE: 2959994, mRNA | 2.30E−05 | 1.08 |
| 424904 | AI221739 | Hs. 96899 | ESTs | 2.08E−03 | 1.08 |
| 439655 | AI925740 | Hs. 91559 | ESTs, Weakly similar to S72576 hypotheti | 2.14E−03 | 1.08 |
| 409026 | AL137554 | Hs. 49927 | protein kinase NYD-SP15 | 9.99E−05 | 1.08 |
| 430715 | AC004908 | | Homo sapiens PAC clone RP5-855D21 | 2.72E−06 | 1.08 |
| 456647 | AI252640 | Hs. 110364 | peptidylprolyl isomerase C (cyclophilin | 4.65E−06 | 1.08 |
| 429261 | AW176254 | Hs. 342389 | ESTs | 3.21E−03 | 1.08 |
| 451813 | NM_16117 | Hs. 27182 | phospholipase A2-activating protein | 7.17E−05 | 1.07 |
| 438117 | AA328041 | Hs. 194329 | hypothetical protein FLJ21174 | 6.88E−07 | 1.07 |
| 451326 | AW296946 | Hs. 256078 | ESTs | 5.35E−05 | 1.07 |
| 425116 | AU076686 | Hs. 154668 | KIAA0391 gene product | 1.18E−03 | 1.07 |
| 451938 | AI354355 | Hs. 16697 | down-regulator of transcription 1, TBP-b | 1.61E−06 | 1.07 |
| 450234 | AI688524 | | ESTs | 2.54E−04 | 1.07 |
| 411739 | AW859623 | Hs. 8262 | lysosomal-associated membrane protein 2 | 1.96E−03 | 1.07 |
| 445372 | N36417 | Hs. 144928 | ESTs | 1.34E−03 | 1.07 |
| 421522 | R48881 | Hs. 102991 | hypothetical protein FLJ13956 | 9.67E−03 | 1.07 |
| 419426 | AI214690 | Hs. 346257 | aldo-keto reductase family 1, member B1 | 2.34E−05 | 1.07 |
| 459126 | AA478866 | Hs. 288809 | hypothetical protein FLJ20159 | 2.40E−04 | 1.07 |
| 417395 | BE564245 | Hs. 82084 | integrin beta 3 binding protein (beta3-e | 9.69E−03 | 1.07 |
| 445133 | AW157646 | Hs. 198689 | ESTs | 3.39E−03 | 1.07 |
| 417688 | R09170 | Hs. 284350 | ESTs | 1.12E−05 | 1.07 |
| 432858 | BE618609 | Hs. 279591 | Homo sapiens clone 25056 mRNA sequence | 3.83E−05 | 1.07 |
| 439363 | AW979183 | Hs. 292414 | ESTs, Weakly similar to I38022 hypotheti | 8.83E−03 | 1.07 |
| 452260 | AA453208 | Hs. 330994 | RAB9, member RAS oncogene family | 4.08E−05 | 1.07 |
| 449838 | AB020653 | Hs. 24024 | KIAA0846 protein | 9.84E−04 | 1.07 |
| 451900 | AB023199 | Hs. 27207 | KIAA0982 protein | 8.47E−07 | 1.07 |
| 450014 | N41322 | Hs. 18441 | ESTs | 2.19E−06 | 1.07 |
| 414257 | AI828600 | Hs. 21124 | ESTs, Weakly similar to ALU8_HUMAN ALU S | 8.09E−05 | 1.07 |
| 424906 | AI566086 | Hs. 153716 | Homo sapiens mRNA for Hmob33 protein, 3′ | 5.69E−04 | 1.07 |
| 404497 | | | Target Exon | 5.11E−04 | 1.07 |
| 448598 | NM_005701 | Hs. 21577 | RNA, U transporter 1 | 4.71E−07 | 1.07 |
| 423767 | H18283 | Hs. 132753 | F-box only protein 2 | 3.06E−04 | 1.07 |
| 454514 | AW802838 | | gb: IL2-UM0076-130500-084-B12 UM0076 Homo | 5.61E−04 | 1.07 |
| 441356 | BE384361 | Hs. 182885 | ESTs, Weakly similar to JC5024 UDP-galac | 6.59E−06 | 1.07 |
| 426378 | U80082 | Hs. 169600 | KIAA0826 protein | 4.00E−04 | 1.07 |
| 428420 | AL096858 | Hs. 184245 | KIAA0929 protein Msx2 interacting nuclea | 8.62E−09 | 1.07 |
| 453827 | AF201948 | Hs. 35660 | BUP protein | 5.87E−07 | 1.07 |
| 454462 | AW754153 | Hs. 155976 | cullin 4B | 5.88E−03 | 1.07 |
| 427120 | R42099 | Hs. 21965 | ESTs | 3.67E−04 | 1.07 |
| 427297 | AW292593 | Hs. 334907 | Homo sapiens, clone MGC: 17333, mRNA, com | 3.42E−06 | 1.07 |
| 408876 | AI961060 | Hs. 129908 | KIAA0591 protein | 4.75E−04 | 1.07 |
| 445929 | AI089660 | Hs. 323401 | dpy-30-like protein | 1.14E−05 | 1.07 |
| 457913 | AA748613 | Hs. 311977 | ESTs, Highly similar to SWI/SNF related, | 4.55E−03 | 1.07 |
| 409278 | AA346683 | Hs. 52763 | anaphase-promoting complex subunit 7 | 1.06E−03 | 1.07 |
| 430939 | AI269471 | Hs. 187018 | ESTs | 5.61E−03 | 1.07 |
| 450943 | AA020964 | Hs. 24734 | oxysterol binding protein | 6.29E−03 | 1.07 |
| 449893 | T97999 | Hs. 18214 | ESTs, Weakly similar to B34087 hypotheti | 1.03E−03 | 1.07 |
| 428821 | H91282 | Hs. 286232 | Homo sapiens cDNA: FLJ23190 fis, clone L | 4.16E−03 | 1.07 |
| 449540 | AA001713 | | gb: zh86e08.s1 Soares_fetal_liver_spleen_{—} | 1.69E−03 | 1.07 |
| 447060 | H79103 | Hs. 270997 | ESTs, Weakly similar to S47072 finger pr | 9.50E−05 | 1.07 |
| 404692 | | | Target Exon | 4.70E−04 | 1.07 |
| 421307 | BE539976 | Hs. 103305 | Homo sapiens mRNA; cDNA DKFZp434B0425 (f | 1.04E−04 | 1.07 |
| 440523 | AW501153 | Hs. 7243 | ubiquitin specific protease 24 | 8.40E−07 | 1.07 |
| 418627 | AL079835 | Hs. 86858 | ribosomal protein S6 kinase, 70 kD, polyp | 2.28E−05 | 1.07 |
| 439427 | AA938150 | Hs. 190534 | ESTs | 4.17E−04 | 1.07 |
| 451923 | BE019040 | Hs. 27258 | calcyclin binding protein | 3.75E−05 | 1.07 |
| 405166 | | | Target Exon | 1.21E−03 | 1.06 |
| 434825 | N87549 | Hs. 125287 | zinc finger protein ZNF140-like protein | 3.48E−06 | 1.06 |
| 425395 | NM_014102 | Hs. 156243 | PRO1848 protein | 1.37E−05 | 1.06 |
| 422008 | AJ000534 | Hs. 110708 | sarcoglycan, epsilon | 1.10E−05 | 1.06 |
| 407656 | AW747986 | Hs. 37443 | Homo sapiens mRNA; cDNA DKFZp434B2119 (f | 1.28E−05 | 1.06 |
| 433935 | AF112208 | Hs. 44163 | 13 kDa differentiation-associated protein | 8.27E−07 | 1.06 |
| 446045 | AV656268 | Hs. 209153 | angiopoietin-like 3 | 7.72E−05 | 1.06 |
| 444156 | AW500059 | | ESTs, Highly similar to AF219140 1 gastr | 1.00E−04 | 1.06 |
| 407621 | AA128805 | Hs. 6815 | Homo sapiens, clone IMAGE: 3626458, mRNA, | 8.90E−07 | 1.06 |
| 409506 | NM_006153 | Hs. 54589 | NCK adaptor protein 1 | 6.83E−03 | 1.06 |
| 458287 | AA987556 | Hs. 12867 | ESTs | 1.25E−03 | 1.06 |
| 454552 | AW806924 | | gb: QV4-ST0023-160400-172-h10 ST0023 Homo | 5.69E−03 | 1.06 |
| 408931 | AA251995 | Hs. 334648 | poly(A) polymerase alpha | 5.04E−03 | 1.06 |
| 417414 | AA434589 | | dUTP pyrophosphatase | 1.69E−04 | 1.06 |
| 405270 | | | NM_018850*: Homo sapiens ATP-binding cass | 5.68E−03 | 1.06 |
| 421544 | AK001445 | Hs. 105633 | hypothetical protein FLJ10583 | 7.06E−04 | 1.06 |
| 449881 | Z28444 | Hs. 24119 | Homo sapiens mRNA; cDNA DKFZp586G2222 (f | 1.68E−04 | 1.06 |
| 450295 | AI766732 | Hs. 210628 | ESTs | 2.38E−03 | 1.06 |
| 413530 | AA130158 | Hs. 19977 | ESTs, Moderately similar to ALU8_HUMAN A | 9.93E−04 | 1.06 |
| 422622 | AW753452 | Hs. 118757 | enhancer of rudimentary (Drosophila) horn | 6.78E−04 | 1.06 |
| 410602 | AI370306 | Hs. 158622 | ESTs | 4.91E−04 | 1.06 |
| 453959 | Z18859 | | guanine nucleotide binding protein (G pr | 4.95E−04 | 1.06 |
| 436503 | AJ277750 | Hs. 183924 | ubiquitin associated and SH3 domain cont | 9.83E−04 | 1.06 |
| 424459 | R56570 | Hs. 50547 | ESTs | 1.03E−03 | 1.06 |
| 433230 | AW136134 | Hs. 220277 | ESTs | 1.55E−05 | 1.06 |
| 453436 | AW504292 | Hs. 193261 | ESTs | 1.05E−04 | 1.06 |
| 459097 | AW062839 | Hs. 154230 | nuclear domain 10 protein | 5.24E−03 | 1.06 |
| 426934 | AI301623 | Hs. 172918 | hypothetical protein | 1.56E−05 | 1.06 |
| 449240 | AF107455 | Hs. 334540 | Homo sapiens, clone IMAGE: 4138787, mRNA, | 3.35E−03 | 1.06 |
| 439195 | H89360 | | gb: yw28d08.s1 Morton Fetal Cochlea Homo | 2.58E−03 | 1.06 |
| 440367 | BE091101 | Hs. 188606 | ESTs | 1.63E−05 | 1.06 |
| 431689 | AA305688 | Hs. 267695 | UDP-Gal: betaGlcNAc beta 1,3-galactosyltr | 8.24E−03 | 1.06 |
| 409598 | NM_014018 | Hs. 55097 | mitochondrial ribosomal protein S28 | 1.16E−06 | 1.06 |
| 402109 | | | Target Exon | 547E−03 | 1.06 |
| 437207 | T27503 | Hs. 15929 | hypothetical protein FLJ12910 | 8.55E−05 | 1.06 |
| 430607 | AW973521 | Hs. 247324 | mitochondrial ribosomal protein S14 | 5.97E−04 | 1.06 |
| 420893 | AK001326 | Hs. 100090 | tetraspan 3 | 1.73E−06 | 1.06 |
| 422868 | AA425589 | Hs. 121076 | peptidylprolyl isomerase (cyclophilin)-l | 1.42E−05 | 1.06 |
| 418977 | AA233094 | Hs. 191517 | ESTs | 2.97E−03 | 1.06 |
| 411388 | X72925 | Hs. 69752 | desmocollin 1 | 1.80E−03 | 1.06 |
| 440417 | AW298070 | Hs. 278675 | ESTs | 1.24E−04 | 1.06 |
| 448780 | W92071 | Hs. 22033 | Human DNA sequence from clone 366N23 on | 6.62E−06 | 1.06 |
| 452405 | AW451241 | Hs. 225641 | hypothetical protein FLJ13171 | 4.88E−07 | 1.06 |
| 439259 | BE464643 | Hs. 242322 | ESTs | 1.85E−03 | 1.05 |
| 423652 | AF052122 | Hs. 130712 | Homo sapiens clone 23929 mRNA sequence | 1.77E−03 | 1.05 |
| 409939 | AA463437 | Hs. 11556 | Homo sapiens cDNA FLJ12566 fis, clone NT | 9.73E−06 | 1.05 |
| 403363 | | | Target Exon | 6.72E−03 | 1.05 |
| 406023 | | | Target Exon | 5.18E−04 | 1.05 |
| 416363 | T78325 | Hs. 81001 | F-box only protein 25 | 6.64E−03 | 1.05 |
| 441736 | AW292779 | Hs. 8182 | ESTs | 2.58E−03 | 1.05 |
| 403185 | | | Target Exon | 5.61E−04 | 1.05 |
| 432538 | BE258332 | Hs. 278362 | male-enhanced antigen | 9.50E−04 | 1.05 |
| 447042 | AB035863 | Hs. 182217 | succinate-CoA ligase, ADP-forming, beta | 1.08E−06 | 1.05 |
| 450631 | AW139773 | Hs. 246827 | ESTs | 8.76E−03 | 1.05 |
| 453195 | BE241876 | Hs. 32352 | hypothetical protein DKFZp434K1210 | 1.16E−04 | 1.05 |
| 407596 | R86913 | Hs. 107622 | gb: yq30f05.r1 Soares fetal liver spleen | 2.86E−03 | 1.05 |
| 431752 | AW337166 | Hs. 43018 | ESTs | 4.94E−04 | 1.05 |
| 445637 | W58459 | Hs. 8949 | hypothetical protein MGC4172 | 4.47E−05 | 1.05 |
| 424285 | BE207168 | Hs. 144630 | nuclear receptor subfamily 2, group F, m | 2.95E−04 | 1.05 |
| 408395 | BE072425 | Hs. 44579 | hypothetical protein FLJ20199 | 8.66E−05 | 1.05 |
| 420825 | AI656727 | Hs. 194657 | gb: tt53f12.x1 NCI_CGAP_GC6 Homo sapiens | 1.85E−05 | 1.05 |
| 414291 | AI289619 | Hs. 13040 | G protein-coupled receptor 86 | 2.40E−06 | 1.05 |
| 406922 | S70284 | Hs. 119597 | gb: stearoyl-CoA desaturase [human, adipo | 2.97E−04 | 1.05 |
| 438805 | AA826048 | Hs. 117887 | ESTs | 8.57E−04 | 1.05 |
| 400223 | | | Eos Control | 1.09E−04 | 1.05 |
| 432332 | AA305351 | Hs. 274369 | uncharacterized hypothalamus protein HAR | 2.30E−03 | 1.05 |
| 413628 | L42542 | Hs. 75447 | ralA binding protein 1 | 1.23E−07 | 1.05 |
| 454067 | AA041455 | | ESTs | 2.09E−04 | 1.05 |
| 442965 | AL134235 | Hs. 222442 | ESTs | 8.21E−03 | 1.05 |
| 422174 | AL049325 | Hs. 112493 | Homo sapiens mRNA; cDNA DKFZp564D036 (fr | 1.08E−03 | 1.05 |
| 431799 | AK000283 | Hs. 270502 | hypothetical protein FLJ20276 | 1.53E−03 | 1.05 |
| 413004 | T35901 | Hs. 75117 | interleukin enhancer binding factor 2, 4 | 1.60E−03 | 1.05 |
| 442092 | AW578669 | | hypothetical protein FLJ12439 | 6.77E−04 | 1.05 |
| 424201 | L33075 | Hs. 1742 | IQ motif containing GTPase activating pr | 2.54E−04 | 1.05 |
| 415673 | BE242249 | Hs. 78592 | eukaryotic translation initiation factor | 4.67E−05 | 1.05 |
| 424909 | S78187 | Hs. 153752 | cell division cycle 25B | 3.66E−05 | 1.05 |
| 432467 | T03667 | Hs. 239388 | Human DNA sequence from clone RP1-304B14 | 3.85E−03 | 1.05 |
| 429623 | NM_005308 | Hs. 211569 | G protein-coupled receptor kinase 5 | 6.02E−03 | 1.05 |
| 403611 | | | NM_017886*: Homo sapiens hypothetical pro | 4.13E−04 | 1.05 |
| 449517 | AW500106 | Hs. 23643 | serine/threonine protein kinase MASK | 8.52E−05 | 1.05 |
| 422480 | AB029016 | Hs. 117333 | Homo sapiens mRNA for KIAA1093 protein, | 8.52E−03 | 1.05 |
| 440719 | AA150869 | Hs. 26267 | ATP-dependant interferon response protei | 6.72E−03 | 1.05 |
| 420520 | AK001978 | Hs. 98510 | similar to rab11-binding protein | 1.06E−04 | 1.05 |
| 409439 | AW390511 | Hs. 288862 | Homo sapiens cDNA: FLJ21260 fis, clone C | 2.58E−03 | 1.05 |
| 430504 | H52761 | | Homo sapiens, clone MGC: 12617, mRNA, com | 9.26E−05 | 1.05 |
| 429750 | AB007916 | Hs. 214646 | KIAA0447 gene product | 3.20E−05 | 1.05 |
| 438887 | R68857 | Hs. 265499 | ESTs | 5.09E−04 | 1.05 |
| 447726 | AL137638 | Hs. 19368 | matrilin 2 | 2.15E−03 | 1.05 |
| 448859 | BE272446 | Hs. 265317 | hypothetical protein MGC2562 | 8.34E−04 | 1.05 |
| 409132 | AJ224538 | Hs. 50732 | protein kinase, AMP-activated, beta 2 no | 9.27E−09 | 1.04 |
| 446838 | AW978336 | Hs. 20999 | nucleolar protein p40; homolog of yeast | 6.42E−05 | 1.04 |
| 426441 | AA297257 | Hs. 169907 | glutathione S-transferase A4 | 4.55E−05 | 1.04 |
| 458096 | AF052138 | Hs. 6580 | Homo sapiens cDNA: FLJ23227 fis, clone C | 6.20E−04 | 1.04 |
| 406731 | AI559131 | | gb: tq31g07.x1 NCI_CGAP_Ut1 Homo sapiens | 3.80E−04 | 1.04 |
| 403707 | | | Target Exon | 9.16E−03 | 1.04 |
| 451572 | AA018556 | Hs. 268691 | ESTs, Moderately similar to ALU2_HUMAN A | 2.08E−04 | 1.04 |
| 434959 | AW974949 | Hs. 186564 | ESTs, Weakly similar to I38022 hypotheti | 8.06E−03 | 1.04 |
| 439802 | R06364 | Hs. 18001 | ESTs | 1.02E−07 | 1.04 |
| 457447 | X78261 | | H. sapiens mRNA for TRE17 5′ extremity an | 1.60E−03 | 1.04 |
| 425866 | BE270097 | Hs. 159971 | SWI/SNF related, matrix associated, acti | 2.57E−03 | 1.04 |
| 416065 | BE267931 | Hs. 78996 | proliferating cell nuclear antigen | 1.96E−07 | 1.04 |
| 426506 | AW935187 | Hs. 170162 | KIAA1357 protein | 7.93E−06 | 1.04 |
| 424088 | AL049942 | Hs. 139240 | DKFZP564F1422 protein | 2.35E−04 | 1.04 |
| 438374 | AA321866 | Hs. 6193 | hypothetical protein FLJ14590 | 4.88E−03 | 1.04 |
| 455833 | BE145360 | Hs. 190064 | ESTs, Weakly similar to I38022 hypotheti | 1.77E−03 | 1.04 |
| 407874 | AI766311 | Hs. 289047 | Homo sapiens cDNA FLJ14059 fis, clone HE | 1.27E−08 | 1.04 |
| 432656 | NM_000246 | Hs. 3076 | MHC class II transactivator | 1.71E−03 | 1.04 |
| 413781 | J05272 | Hs. 850 | IMP (inosine monophosphate) dehydrogenas | 6.46E−03 | 1.04 |
| 449865 | AW204272 | Hs. 199371 | ESTs | 1.78E−08 | 1.04 |
| 452420 | BE564871 | Hs. 29463 | centrin, EF-hand protein, 3 (CDC31 yeast | 1.33E−06 | 1.04 |
| 423314 | AI400661 | Hs. 127811 | disintegrin metalloproteinase with throm | 1.34E−03 | 1.04 |
| 443303 | U67319 | Hs. 9216 | caspase 7, apoptosis-related cysteine pr | 1.41E−06 | 1.04 |
| 456175 | BE241702 | Hs. 79402 | polymerase (RNA) II (DNA directed) polyp | 8.45E−03 | 1.04 |
| 420179 | N74530 | Hs. 21168 | ESTs | 7.87E−04 | 1.04 |
| 410209 | AI583661 | Hs. 60548 | hypothetical protein PRO1635 | 5.97E−04 | 1.04 |
| 434148 | AW291716 | Hs. 116113 | Homo sapiens cDNA FLJ14182 fis, clone NT | 1.60E−03 | 1.04 |
| 459274 | AA382590 | Hs. 46366 | KIAA0948 protein | 1.05E−06 | 1.04 |
| 450848 | AI677994 | Hs. 428 | fms-related tyrosine kinase 3 ligand | 5.70E−04 | 1.04 |
| 411580 | AL080088 | Hs. 70877 | DKFZP564K2062 protein | 3.19E−06 | 1.04 |
| 436523 | BE612990 | Hs. 5212 | single-strand selective monofunctional u | 3.67E−03 | 1.04 |
| 428137 | AA421792 | Hs. 170999 | ESTs | 8.38E−04 | 1.04 |
| 426494 | AL119528 | Hs. 170098 | KIAA0372 gene product | 1.22E−03 | 1.04 |
| 434805 | AW974666 | Hs. 293024 | ESTs | 3.83E−03 | 1.04 |
| 455679 | BE066529 | | gb: RC3-BT0333-300300-017-a12 BT0333 Homo | 5.56E−04 | 1.04 |
| 437739 | AW579216 | Hs. 264610 | ESTs, Moderately similar to Ibd1 [H. sapi | 3.46E−05 | 1.04 |
| 412672 | AA158910 | Hs. 74441 | chromodomain helicase DNA binding protei | 2.77E−03 | 1.04 |
| 447749 | T53260 | Hs. 191091 | ESTs | 4.48E−05 | 1.04 |
| 452461 | N78223 | Hs. 108106 | transcription factor | 7.56E−04 | 1.04 |
| 447915 | AW450650 | Hs. 20013 | GCIP-interacting protein p29 | 1.82E−05 | 1.04 |
| 447030 | AW444659 | Hs. 232184 | ESTs | 9.89E−03 | 1.04 |
| 430919 | AA489041 | Hs. 295448 | ESTs | 1.01E−03 | 1.04 |
| 406684 | X16354 | Hs. 50964 | carcinoembryonic antigen-related cell ad | 4.34E−04 | 1.03 |
| 433882 | U90441 | Hs. 3622 | procollagen-proline, 2-oxoglutarate 4-di | 2.44E−04 | 1.03 |
| 430534 | AA480908 | | gb: aa28f09.s1 NCI_CGAP_GCB1 Homo sapiens | 3.51E−03 | 1.03 |
| 413010 | AA393273 | Hs. 75133 | transcription factor 6-like 1 (mitochond | 1.08E−05 | 1.03 |
| 409490 | AA075064 | | gb: zm85e12.s1 Stratagene ovarian cancer | 2.11E−03 | 1.03 |
| 422231 | AA443512 | Hs. 101383 | ESTs | 3.11E−04 | 1.03 |
| 425376 | H10612 | | gb: ym08f03.r1 Soares infant brain 1NIB H | 2.49E−03 | 1.03 |
| 434733 | AI334367 | Hs. 159337 | ESTs | 1.56E−04 | 1.03 |
| 425973 | BE379570 | Hs. 165439 | arsA (bacterial) arsenite transporter, A | 1.41E−03 | 1.03 |
| 458477 | NM_000314 | Hs. 10712 | phosphatase and tensin homolog (mutated | 2.73E−04 | 1.03 |
| 440708 | AF038962 | Hs. 7381 | voltage−dependent anion channel 3 | 1.07E−04 | 1.03 |
| 412338 | AA151527 | Hs. 69485 | hypothetical protein FLJ12436 | 1.38E−05 | 1.03 |
| 424277 | AA338057 | Hs. 29131 | nuclear receptor coactivator 2 | 1.94E−03 | 1.03 |
| 427625 | AF008216 | Hs. 285013 | putative human HLA class II associated p | 2.54E−03 | 1.03 |
| 410017 | AW952426 | Hs. 109438 | Homo sapiens clone 24775 mRNA sequence | 6.00E−06 | 1.03 |
| 404124 | | | Target Exon | 6.53E−04 | 1.03 |
| 446895 | AA166655 | Hs. 26860 | ESTs | 8.18E−05 | 1.03 |
| 448848 | AF131851 | Hs. 22241 | hypothetical protein | 1.70E−03 | 1.03 |
| 403292 | | | Target Exon | 4.58E−03 | 1.03 |
| 425728 | T84535 | Hs. 303001 | Homo sapiens cDNA: FLJ21335 fis, clone C | 3.27E−03 | 1.03 |
| 437440 | AA846804 | | ESTs | 1.45E−03 | 1.03 |
| 433486 | AW976937 | Hs. 54452 | ESTs, Weakly similar to T00329 hypotheti | 7.37E−06 | 1.03 |
| 445790 | AV655170 | Hs. 49015 | chromosome 21 open reading frame 35 | 4.70E−03 | 1.03 |
| 424139 | D86979 | Hs. 141296 | KIAA0226 gene product | 1.13E−05 | 1.03 |
| 451658 | AW195351 | Hs. 250520 | ESTs, Moderately similar to I38022 hypot | 7.97E−06 | 1.03 |
| 458409 | AV645567 | Hs. 120391 | ESTs | 7.85E−04 | 1.03 |
| 425210 | AA054679 | Hs. 155150 | ribonuclease P (14 kD) | 1.78E−03 | 1.03 |
| 429762 | AI346255 | Hs. 216354 | ring finger protein 5 | 5.43E−06 | 1.03 |
| 449578 | AL050331 | Hs. 284141 | KIAA0721 protein | 5.02E−06 | 1.03 |
| 440244 | AI743977 | Hs. 205144 | ESTs | 5.07E−04 | 1.03 |
| 453784 | AA456246 | Hs. 271541 | Homo sapiens cDNA FLJ11653 fis, clone HE | 3.05E−04 | 1.03 |
| 453327 | AW500180 | | tryptophanyl-tRNA synthetase | 1.81E−05 | 1.03 |
| 422298 | NM_005683 | Hs. 114545 | G protein-coupled receptor 55 | 1.63E−04 | 1.03 |
| 428384 | M54968 | | v-Ki-ras2 Kirsten rat sarcoma 2 viral on | 7.06E−04 | 1.03 |
| 447982 | H22953 | Hs. 137551 | ESTs | 1.77E−05 | 1.03 |
| 436911 | AA142984 | Hs. 5344 | adaptor-related protein complex 1, gamma | 1.96E−03 | 1.03 |
| 421873 | AI132988 | Hs. 109052 | chromosome 14 open reading frame 2 | 2.16E−05 | 1.03 |
| 432221 | M21191 | Hs. 273415 | aldolase A, fructose-bisphosphate | 4.65E−03 | 1.03 |
| 403027 | | | C21000364*: gi\|8394509\|ref\|NP_058778.1\|u | 2.37E−04 | 1.03 |
| 449845 | AW971183 | Hs. 6019 | DnaJ (Hsp40) homolog, subfamily C, membe | 1.48E−05 | 1.03 |
| 447221 | AB007861 | Hs. 17803 | KIAA0401 protein | 1.88E−05 | 1.03 |
| 419725 | U66048 | Hs. 92683 | Homo sapiens clone 161455 breast express | 1.11E−05 | 1.03 |
| 456863 | T16837 | Hs. 4241 | ESTs | 1.83E−05 | 1.03 |
| 430599 | NM_004855 | Hs. 247118 | phosphatidylinositol glycan, class B | 1.54E−04 | 1.03 |
| 426295 | AW367283 | | zinc finger protein 6 (CMPX1) | 1.72E−06 | 1.03 |
| 414312 | AA155694 | Hs. 191060 | ESTs | 1.81E−04 | 1.03 |
| 428484 | AF104032 | Hs. 184601 | solute carrier family 7 (cationic amino | 9.02E−08 | 1.03 |
| 452019 | AL157503 | Hs. 27552 | Homo sapiens mRNA; cDNA DKFZp586N2424 (f | 6.94E−04 | 1.02 |
| 445896 | W37944 | Hs. 4007 | Sarcolemmal-associated protein | 8.01E−04 | 1.02 |
| 452606 | N45202 | Hs. 90012 | hypothetical protein FLJ23441 | 9.66E−04 | 1.02 |
| 438940 | AF075045 | Hs. 271609 | ESTs | 5.84E−04 | 1.02 |
| 436685 | W28661 | Hs. 5288 | Homo sapiens mRNA; cDNA DKFZp434M245 (fr | 1.76E−05 | 1.02 |
| 407233 | X16354 | Hs. 50964 | carcinoembryonic antigen-related cell ad | 6.24E−04 | 1.02 |
| 432021 | AA524470 | Hs. 58753 | ESTs | 1.30E−04 | 1.02 |
| 444239 | R57988 | Hs. 10706 | epithelial protein lost in neoplasm beta | 2.69E−03 | 1.02 |
| 444461 | R53734 | Hs. 25978 | ESTs, Weakly similar to 2109260A B cell | 4.54E−03 | 1.02 |
| 439467 | AW292275 | Hs. 158365 | ESTs | 4.93E−03 | 1.02 |
| 417713 | D42047 | Hs. 82432 | KIAA0089 protein | 7.09E−04 | 1.02 |
| 443968 | AA287702 | Hs. 10031 | KIAA0955 protein | 2.40E−03 | 1.02 |
| 411400 | AA311919 | Hs. 69851 | nucleolar protein family A, member 1 (H/ | 1.09E−05 | 1.02 |
| 406535 | | | Target Exon | 2.33E−05 | 1.02 |
| 426007 | AA724983 | Hs. 144743 | ESTs, Weakly similar to AD08_HUMAN ADAM | 4.26E−04 | 1.02 |
| 408103 | AA992592 | Hs. 42712 | MAX protein | 1.83E−08 | 1.02 |
| 436024 | AI800041 | | ESTs | 1.16E−05 | 1.02 |
| 448393 | AW117691 | Hs. 224517 | ESTs, Moderately similar to hypothetical | 5.08E−04 | 1.02 |
| 410277 | R88621 | Hs. 26249 | ESTs, Weakly similar to T2D3_HUMAN TRANS | 2.62E−03 | 1.02 |
| 443985 | BE613332 | Hs. 132055 | ESTs, Weakly similar to GNMSLL retroviru | 5.17E−03 | 1.02 |
| 420168 | AF217508 | Hs. 95594 | serine carboxypeptidase vitellogenic-lik | 7.46E−06 | 1.02 |
| 458649 | AI291047 | Hs. 49053 | ESTs | 1.64E−03 | 1.02 |
| 439462 | AL133026 | Hs. 6567 | Homo sapiens mRNA; cDNA DKFZp434C136 (fr | 1.26E−04 | 1.02 |
| 439871 | R88518 | Hs. 46736 | hypothetical protein FLJ23476 | 5.31E−03 | 1.02 |
| 443724 | AA446783 | Hs. 288932 | hypothetical protein FLJ13194 | 1.35E−03 | 1.02 |
| 408627 | BE313882 | Hs. 46659 | DKFZP58612223 protein | 3.10E−03 | 1.02 |
| 443291 | AA325633 | Hs. 136102 | KIAA0853 protein | 4.07E−04 | 1.02 |
| 441243 | AI767056 | Hs. 193002 | ESTs | 1.55E−03 | 1.02 |
| 419881 | AA329340 | | mannosyl (alpha-1,3-)-glycoprotein beta- | 3.38E−05 | 1.02 |
| 438081 | H49546 | Hs. 251391 | claudin 16 | 3.84E−03 | 1.02 |
| 423456 | AL110151 | Hs. 128797 | DKFZP586D0824 protein | 3.80E−06 | 1.02 |
| 422090 | W05345 | Hs. 293884 | ESTs | 1.52E−05 | 1.02 |
| 433920 | NM_000430 | Hs77318 | platelet-activating factor acetylhydrola | 2.37E−04 | 1.02 |
| 411142 | NM_014256 | Hs. 69009 | transmembrane protein 3 | 7.98E−04 | 1.02 |
| 437119 | AI379921 | Hs. 177043 | ESTs | 5.38E−04 | 1.02 |
| 414022 | W44916 | | myosin regulatory light chain 2, smooth | 2.93E−03 | 1.02 |
| 415524 | F11541 | | gb: HSC2YD011 normalized infant brain cDN | 5.94E−03 | 1.02 |
| 442119 | AW970826 | Hs. 6185 | KIAA1557 protein | 2.43E−03 | 1.02 |
| 440865 | AI281525 | Hs. 130180 | ESTs | 2.56E−03 | 1.02 |
| 449227 | AJ403106 | Hs. 121824 | ESTs | 3.98E−03 | 1.02 |
| 426224 | BE085860 | Hs. 168075 | karyopherin (importin) beta 2 | 9.60E−07 | 1.02 |
| 435272 | AA906415 | Hs. 110041 | ESTs | 2.06E−03 | 1.02 |
| 449269 | AI564682 | Hs. 175870 | ESTs | 3.26E−05 | 1.02 |
| 438685 | AA814034 | Hs. 123581 | ESTs | 1.79E−03 | 1.02 |
| 422558 | NM_006420 | Hs. 118249 | brefeldin A-inhibited guanine nucleotide | 5.07E−05 | 1.02 |
| 441244 | BE612935 | Hs. 184052 | PP1201 protein | 1.09E−04 | 1.02 |
| 420473 | AL118782 | Hs. 300208 | Sec23-interacting protein p125 | 2.28E−03 | 1.02 |
| 445504 | NM_006590 | Hs. 12820 | SnRNP assembly defective 1 homolog | 4.35E−03 | 1.02 |
| 437952 | D63209 | Hs. 5944 | solute carrier family 11 (proton-coupled | 7.25E−09 | 1.02 |
| 431109 | AA492271 | Hs. 143672 | ESTs | 3.55E−05 | 1.02 |
| 418535 | R98238 | Hs. 268874 | ESTs, Weakly similar to ZN91_HUMAN ZINC | 2.05E−03 | 1.02 |
| 425245 | AI751768 | Hs. 155314 | KIAA0095 gene product | 1.68E−05 | 1.02 |
| 445903 | AI347487 | Hs. 132781 | class I cytokine receptor | 4.56E−04 | 1.02 |
| 445558 | R99105 | Hs. 173522 | ESTs | 3.62E−03 | 1.01 |
| 410625 | AI753267 | Hs. 15220 | zinc finger protein 106 | 5.62E−03 | 1.01 |
| 409162 | H25530 | Hs. 50868 | solute carrier family 22 (organic cation | 1.30E−04 | 1.01 |
| 411990 | AW963624 | Hs. 31707 | ESTs, Weakly similar to YEW4_YEAST HYPOT | 2.38E−04 | 1.01 |
| 419497 | NM_006410 | Hs. 90753 | Tat-interacting protein (30 kD) | 9.78E−05 | 1.01 |
| 428974 | AA442693 | Hs. 272006 | ESTs, Weakly similar to I38022 hypotheti | 3.99E−07 | 1.01 |
| 435014 | BE560898 | Hs. 10026 | mitochondrial ribosomal protein L17 | 3.50E−03 | 1.01 |
| 430268 | AK000737 | Hs. 237480 | hypothetical protein FLJ20730 | 1.21E−05 | 1.01 |
| 408340 | AB037762 | Hs. 44268 | myelin gene expression factor 2 | 2.84E−06 | 1.01 |
| 410576 | AI421612 | Hs. 30864 | ESTs | 3.10E−03 | 1.01 |
| 444187 | AW138466 | Hs. 151274 | ESTs | 1.63E−03 | 1.01 |
| 430487 | D87742 | Hs. 241552 | KIAA0268 protein | 1.29E−05 | 1.01 |
| 431214 | AA294921 | Hs. 348024 | v-ral simian leukemia viral oncogene hom | 4.89E−04 | 1.01 |
| 428592 | AA479487 | Hs. 105642 | Homo sapiens cDNA: FLJ23271 fis, clone H | 1.27E−04 | 1.01 |
| 412938 | AU077050 | Hs. 75066 | translin | 3.21E−06 | 1.01 |
| 414355 | BE272157 | Hs. 75925 | proteasome (prosome, macropain) inhibito | 4.98E−05 | 1.01 |
| 424026 | AI798295 | Hs. 137576 | ribosomal protein L34 pseudogene 1 | 2.24E−04 | 1.01 |
| 432975 | AA331517 | Hs. 286055 | chimerin (chimaerin) 2 | 5.03E−03 | 1.01 |
| 419170 | BE002798 | Hs. 287850 | integral membrane protein 1 | 3.08E−04 | 1.01 |
| 459428 | AA258989 | | ESTs | 5.76E−03 | 1.01 |
| 451579 | AW607731 | Hs. 26670 | Human PAC clone RP3-515N1 from 22q11.2-q | 4.45E−06 | 1.01 |
| 408128 | N72794 | Hs. 37716 | ESTs, Weakly similar to ataxin-2-like pr | 1.94E−03 | 1.01 |
| 412634 | U55984 | | heat shock 90 kD protein 1, alpha | 9.53E−05 | 1.01 |
| 425620 | AA447450 | Hs. 124219 | hypothetical protein FLJ12934 | 3.30E−03 | 1.01 |
| 421829 | AB018330 | Hs. 108708 | calcium/calmodulin-dependent protein kin | 4.76E−05 | 1.01 |
| 434552 | AA639618 | Hs. 325116 | Homo sapiens, clone MGC: 2962, mRNA, comp | 3.66E−03 | 1.01 |
| 426348 | BE466586 | Hs. 17433 | hypothetical protein FLJ20967 | 1.18E−04 | 1.01 |
| 458984 | AA011185 | Hs. 221189 | hypothetical protein FLJ14431 | 3.93E−04 | 1.01 |
| 420112 | NM_005109 | Hs. 95220 | oxidative-stress responsive 1 | 4.32E−03 | 1.01 |
| 437142 | AI791617 | Hs. 145068 | ESTs, Moderately similar to A46010 X-lin | 2.45E−04 | 1.01 |
| 410913 | AL050367 | Hs. 66762 | Homo sapiens mRNA; cDNA DKFZp564A026 (fr | 6.41E−05 | 1.01 |
| 422749 | W01076 | Hs. 278573 | CD59 antigen p18-20 (antigen identified | 7.76E−03 | 1.01 |
| 456926 | AB018284 | Hs. 158688 | KIAA0741 gene product | 5.40E−06 | 1.01 |
| 444745 | AF117754 | Hs. 11861 | thyroid hormone receptor-associated prot | 8.02E−05 | 1.01 |
| 416721 | H80001 | | gb: ys65d09.r1 Soares retina N2b4HR Homo | 4.32E−05 | 1.01 |
| 425218 | NM_014909 | Hs. 155182 | KIAA1036 protein | 4.15E−03 | 1.01 |
| 415724 | NM_003580 | Hs. 78687 | neutral sphingomyelinase (N-SMase) activ | 3.02E−06 | 1.01 |
| 419013 | T90378 | Hs. 14463 | ESTs | 2.26E−03 | 1.01 |
| 440461 | R52728 | Hs. 7193 | KIAA1183 protein | 4.24E−04 | 1.01 |
| 445760 | AW370515 | Hs. 13273 | KIAA0592 protein | 9.19E−07 | 1.01 |
| 422732 | AA577455 | Hs. 24937 | transformer-2 alpha (htra-2 alpha) | 3.04E−04 | 1.01 |
| 453528 | AL137459 | Hs. 33104 | Huntingtin interacting protein C | 5.40E−05 | 1.01 |
| 454900 | AW838129 | | gb: QV2-LT0051-240300-097-g12 LT0051 Homo | 7.63E−03 | 1.01 |
| 421395 | D90084 | Hs. 1023 | pyruvate dehydrogenase (lipoamide) alpha | 3.22E−04 | 1.01 |
| 414662 | AL036058 | Hs. 76807 | major histocompatibility complex, class | 6.04E−04 | 1.01 |
| 433505 | AW504027 | Hs. 15301 | Homo sapiens cDNA FLJ12596 fis, clone NT | 2.97E−05 | 1.01 |
| 431712 | R26584 | Hs. 267993 | hypothetical protein FLJ10143 | 2.30E−05 | 1.01 |
| 432145 | AB040890 | Hs. 272759 | KIAA1457 protein | 1.58E−03 | 1.01 |
| 432878 | BE386490 | Hs. 279663 | Pirin | 3.32E−06 | 1.01 |
| 412025 | AI827451 | Hs. 24143 | Wiskott-Aldrich syndrome protein interac | 8.61E−06 | 1.00 |
| 408988 | AL119844 | Hs. 49476 | Homo sapiens clone TUA8 Cri-du-chat regi | 5.19E−04 | 1.00 |
| 430408 | AA478540 | Hs. 108336 | ESTs, Weakly similar to ALUE_HUMAN !!!! | 1.07E−05 | 1.00 |
| 419021 | AI908330 | Hs. 10554 | hypothetical protein FLJ12761 | 2.11E−07 | 1.00 |
| 447188 | H65423 | Hs. 17631 | hypothetical protein DKFZp434E2135 | 6.76E−04 | 1.00 |
| 409065 | AB033113 | Hs. 50187 | KIAA1287 protein | 1.25E−04 | 1.00 |
| 410868 | T06529 | Hs. 98518 | Homo sapiens cDNA FLJ11490 fis, clone HE | 5.88E−04 | 1.00 |
| 442462 | AF031405 | | gb: AF031405 Soares fetal liver spleen 1N | 1.37E−03 | 1.00 |
| 434665 | AA642125 | | gb: nr60c01.s1 NCI_CGAP_Lym3 Homo sapiens | 8.06E−03 | 1.00 |
| 435869 | AF255910 | Hs. 54650 | junctional adhesion molecule 2 | 8.84E−04 | 1.00 |
| 405922 | | | Target Exon | 2.64E−03 | 1.00 |
| 400239 | | | NM_001610: Homo sapiens acid phosphatase | 2.26E−04 | 1.00 |
| 419632 | AK000155 | Hs. 91684 | Homo sapiens mRNA; cDNA DKFZp667I103 (fr | 5.91E−03 | 1.00 |
| 431857 | W19144 | Hs. 271742 | ADP-ribosyltransferase (NAD; poly (ADP-r | 8.59E−06 | 1.00 |
| 438543 | AA810141 | Hs. 192182 | ESTs | 2.40E−06 | 1.00 |
| 458430 | AV646505 | Hs. 122155 | ESTs | 6.08E−03 | 1.00 |
| 445106 | T10219 | Hs. 12329 | KIAA0697 protein | 2.01E−04 | 1.00 |
| 422926 | NM_016102 | Hs. 121748 | ring finger protein 16 | 3.85E−03 | 1.00 |
| 443956 | Z45647 | Hs. 107968 | ESTs | 3.22E−03 | 1.00 |
| 453128 | AW026516 | Hs. 31791 | acylphosphatase 2, muscle type | 2.24E−04 | 1.00 |
| 431195 | AA503083 | Hs. 79742 | ESTs, Weakly similar to T29959 DNA-direc | 1.99E−03 | 1.00 |
| 447348 | AI270615 | Hs. 18192 | Ser/Arg-related nuclear matrix protein ( | 2.85E−04 | 1.00 |
| 455148 | AW858403 | | gb: CM0-CT0341-181299-130-f04 CT0341 Homo | 4.78E−03 | 1.00 |
| 434775 | AA648983 | | ESTs | 2.20E−05 | 1.00 |
| 433456 | AA593447 | Hs. 124296 | ESTs | 1.75E−03 | 1.00 |
| 410800 | BE280421 | Hs. 94499 | ESTs | 5.76E−05 | 1.00 |
| 447370 | AW248150 | Hs. 18349 | mitochondrial ribosomal protein L15 | 1.29E−05 | 1.00 |
| 406238 | | | Target Exon | 1.03E−03 | 1.00 |

Pkey: Unique Eos probeset identifier number
ExAccn: Exemplar Accession number, Genbank accession number
UnigeneID: Unigene number
Unigene Title: Unigene gene title
R1: T-test p-value
R2: Difference of the average standardized value of Hep C+ Liver vs. the average standardized value of Hep C− Liver

TABLE 11B

| | CAT | |
|---|---|---|
| Pkey | Number | Accession |
| 414404 | 19538_5 | BG036308 AW813969 AW579686 BE147476 AW813871 BE147474 BE147589 BE147582 AA767716 AW813960 AW813958 |
| | | BE147583 |
| | | AW813608 AW813612 AW813965 AW813954 BE706154 BF843546 AW813870 BE147801 AW813962 R80512 BG681408 |
| | | AA344564 AW370224 |
| | | BG614838 AW582118 AW582114 AW370227 W16712 BF209670 BG117739 BE762802 BE087964 BE888647 R89145 |
| | | BF342601 BI752591 |
| | | BG435038 AA166732 N79515 BF541709 N79567 BG503029 |
| 455705 | 77478_2 | BF971018 BE513812 AA133359 AW581719 BF434402 AL600619 BG699731 BI551395 AW027136 AW055130 BF939512 |
| | | AI076048 H18584 |
| | | AW161061 AA864334 AI816101 BE049456 AW044012 AA954079 AI274682 AI370526 AW131990 AA853195 AA853191 |
| | | BG118295 AA761620 |
| | | BG705371 BF355591 BF336596 AA360497 H28072 BG198352 AW364709 H40926 H44214 AA836538 BI059563 |
| 437938 | 66997_1 | U71456 AA482911 W78802 AW856538 BF737212 N36809 N35320 AA282915 AW505512 AI653832 W87891 AI961530 |
| | | T85904 H59397 R97278 |
| | | W01059 AI820532 T82391 AI820501 T63226 R66056 R67840 AW961101 AA337499 W37181 AA180009 AW205862 |
| | | AA988777 AA856975 |
| | | BF172457 BG751124 AI741346 AI950344 AI689062 AI872193 AW102898 AW173586 AI763273 AI890387 AW150329 |
| | | AI762688 AA488892 |
| | | AI356394 AI539642 AA642789 AI950087 BF589902 N70208 AA283144 AA488964 H60052 R97040 BF886630 AW967677 |
| | | AW971573 AW967671 |
| | | AI308119 AA251875 AA908598 AI819225 AI564269 AA908741 AA293273 AA969759 AW276905 AA044209 H83488 |
| | | T92487 |
| 452194 | 90339_1 | AI694413 AW994700 AI912946 N73548 AI082035 AW271652 W24189 W24182 AI719718 AA024658 |
| | | AW810120 AW015394 |
| | | T79755 AA988043 |
| | | AI709339 |
| 437834 | 294580_1 | BG110129 AW749287 BE535498 AW749299 AW749293 AW749302 AW749298 AW749291 AW749294 |
| | | AW749289 AW749288 |
| | | AW749296 |
| | | AA769294 AW749297 AW749295 AW749292 BE002573 |
| 419200 | 9531_1 | BF036043 AW190446 BG194731 AW662036 AI445021 BE937550 AW818972 AW393132 AA834685 BF112058 AV721682 |
| | | H16423 AI270167 |
| | | AI857345 AA937302 AW818444 BE929780 BG498678 BF155010 BI598271 BI599811 BE161728 AW578737 AW753711 |
| | | AW379707 AW381918 |
| | | BG506608 AW028637 AW994240 BF887392 BF790073 AW381624 AV727105 BF439618 AA443174 AI018009 N42850 |
| | | AW573242 AI417258 |
| | | AA463483 AI676131 AI167170 AA836627 AA443828 AW592922 AA235129 AA730278 AW439062 AW474332 BI043239 |
| | | AW474342 BG708553 |
| | | AW362423 BF090028 BE827256 R16550 R39478 R39479 R94368 BG540916 BM314745 AA251087 D54231 D55274 |
| | | BF085805 D31589 |
| | | AW966405 AW994425 D81879 BE093545 AW901107 AA383529 BI021552 R56420 N39976 AA573281 H82595 AA234955 |
| | | BE093539 AW367006 |
| | | BF358697 BF366318 AA663856 BE702099 BF035969 AI267384 AI267232 BE348320 AA621574 AA861212 BF083343 |
| | | BF083341 AV745131 |
| | | D53074 AW954476 AW954472 AA376836 AV724531 D53063 C14928 AA093287 AA062638 BG483558 BE940050 |
| | | AA765954 |
| | | T70171 BE938775 |
| | | BE940057 053502 AW373300 AL118798 BM128728 AA193411 AW444709 AW952455 AI887612 BF431948 BI496876 |
| | | AI264159 BM128481 |
| | | AI624657 AI689301 AI969467 AA861685 AA251595 AA625761 AA872090 AI826790 AA328366 BE827416 R75951 |
| | | D56918 R68122 BE827384 |
| | | AL118797 AI184164 AA164411 BI495332 BE858113 AI863860 H00660 T69849 AW780389 C14667 BE934995 |
| | | BI018652 R92801 AA164410 |
| | | H00752 AW373305 AW373299 AW373302 |
| 434210 | 54921_1 | AK057015 AI026834 BE857936 AA149091 AI742972 AW439172 AI253168 AA255613 |
| | | BF513175 AI005006 T03406 |
| | | AW338149 AA836442 |
| | | AA420530 R88566 AI611672 AA433916 AA442855 BF063008 AA812568 AI889706 AA715313 AA768539 AA767620 |
| | | AA665471 AA404380 |
| | | AA665612 BF056442 AA706388 AI650676 AA627448 AI141769 H78227 AW901852 H78221 BE701982 BF689273 |
| | | AA397464 |
| | | N33072 R60218 |
| | | AW968247 H14833 AA768305 AA043348 R56470 BF739832 R51827 AI474963 BG494574 AA149090 BF238154 AI802210 |
| | | BE000129 BF734513 |
| | | R41964 H21055 R85253 R17705 R40844 BF790218 BG388356 BF003037 AA703138 AA377348 W24822 |
| 416126 | 12435_2 | AK057910 H24547 R46556 BI043624 H20063 R66415 |
| 406906 | | |
| 400244 | 12188_1 | X79449 BC017853 AL121035 BF196384 AW119044 AI028023 AW451110 AI971911 |
| | | AW015069 AI079170 AI376367 |
| | | AI264113 AA829646 |
| | | AA737579 AA449679 AA740864 NM_001111 U18121 AL567297 BG773801 BF973874 AV687104 AA527579 |
| | | AA843525 BE706355 AI074589 |
| | | AI523475 BE890249 AW406263 BE074258 AV729485 BF809610 BG058619 AA677244 BE179838 AA622264 AI460106 |
| | | AA740411 AI499168 |
| | | AI078223 AI682923 BE696559 AW375385 AA788739 BG984978 240874 T17054 F09669 AW844043 U10439 BI711870 |
| | | AW245957 AU158567 |
| | | AA679305 AA679316 W72510 AI346029 BG059762 AW251062 AA132373 AI925621 AI860230 AI340172 AW192891 |
| | | AI707980 AI094937 |
| | | AI042115 AI200901 BE328452 AA644678 AA551209 BE351065 AA970761 N68609 AW002028 AA160826 AI422774 |
| | | AW873114 AW073597 |
| | | AW664483 AI218710 AW020550 AW190607 AI984545 AI871921 AI333970 AI452887 AI818335 AA398655 AI554424 |
| | | AI274187 BE465703 |
| | | AW512940 AW241366 AI923954 AA576649 AW168294 AA813181 AA912168 AI049738 AW514073 AA548255 AI569630 |
| | | BE710031 AA244182 |
| | | AI341697 AA563904 AI537990 AW517908 AW172943 Z39498 AI750294 AW150414 AI253293 BE825720 T31860 |
| | | AW150775 |
| | | D20310 AA150892 |
| | | AU133933 BE781148 AL038957 BF910979 AA352297 BG988142 AW372175 BF229106 AW866705 BE093482 BG990396 |
| | | AI499917 AA054452 |
| | | H05484 AI828502 BM467331 AU140570 AL135417 BF947202 AW391926 BE813418 BF998473 T92021 BI021048 |
| | | BM048783 AW501366 |
| | | AW501342 W501549 E939021 BE707147 BE160974 BE305207 N49011 AA947119 AA678801 BE536876 AW897428 |
| | | BG329648 BG818540 |
| | | BE542344 BI919250 BI253018 AW130996 BE074249 BE895428 BI034862 BE083277 BF952166 |
| 411837 | 1110600_1 | AW866303 AW866402 AW866310 AW866282 |
| 406990 | 48529_1 | S82075 AA327293 |
| 433489 | 1000815_1 | AA594718 AI698673 AA907314 AW629150 |
| 411605 | 10026_3 | BG256892 H10532 N46614 R52610 AW977696 BM460488 W56819 BI042183 BG977498 BE767451 BF870009 BG477472 |
| | | R61137 R14274 |
| | | R20259 R09686 BI838226 BF034269 AA429173 BE741829 AW867495 AI123683 AW006831 BE831162 AW452753 |
| | | AV742717 W86152 |
| | | BF115102 AI633815 BF921562 AA094230 BE092587 W86151 AA526153 AI672156 BF914496 R12579 BF852352 AA699780 |
| | | T57386 BF903022 R09933 AA678298 |
| 420292 | 2556_2 | AW991897 AW991891 AW991894 AW991841 AW991840 AW991777 AW991797 AW991779 AW991766 |
| | | AW991774 AW991793 AW991786 |
| | | AW991782 AW991773 AW991783 AW991828 BF326561 AW991887 AW991888 AW991896 AW991901 AW991893 |
| | | AW991898 AW991895 |
| | | AW991902 AW991899 BF326532 AW991727 AW991742 AW991731 AW991738 AW991736 AW991739 AW991740 |
| | | AW991729 AW991741 |
| | | AW991730 AW991733 AW991804 AW991803 AW991808 AW991805 AW991801 AW991806 AW991802 AW991798 |
| | | AW991728 AW991807 |
| | | AW991776 AW991714 BF326565 BF326506 AW991726 AW991734 AW991732 BF326535 AW991800 AW991809 |
| | | BF326568 AW991900 |
| | | BF326518 BF326524 AW991716 AW991795 AW991718 |
| 442679 | 31783_3 | BG621493 BI056706 BG496376 R53718 W65356 R79357 BG434247 AA357769 AW978686 BG573200 BF132113 |
| | | BF086709 AA366938 D79234 |
| | | BG494628 AA156754 BG434311 AW978683 AW273417 BM054662 AI799886 AI433351 AI160798 AW33742 |
| | | BF056186 AI281606 AW015046 |
| | | AI439585 AI245530 AI078267 AA807170 AA837395 W61252 AA831085 AA287371 AW768354 AA890606 AI302539 |
| | | AI708575 AI673031 |
| | | AI242260 AW514069 AA283958 AA825452 AI371234 AA425696 AA453422 AA827697 R23653 D20240 AA772517 H13802 |
| | | R66972 R79360 |
| | | R27351 F03379 AA031952 N69504 R33143 R79358 R39136 R38800 R15089 R52937 R37502 H01021 R33634 R46551 |
| | | Z40404 BG291052 |
| | | BG570357 AW391046 BG496872 H23558 |
| 446526 | 11131_17 | AK023419 BE464935 BE221668 AI148885 BI859909 AI374780 AA766892 AI347967 AI582917 AA477117 |
| | | AA229236 AA652637 AA636109 |
| | | AU134580 F21298 BF802607 BE769124 AV658891 H89616 AV659853 |
| 451752 | 10408_5 | AB032997 AI141678 AW978722 BE467119 AI761408 BF727385 AW237035 AI934521 BF436248 AI479668 |
| | | Z40632 AA832081 |
| | | AW295901 |
| | | BF057835 BE465977 AI621269 BE465983 BF756369 N74056 AI817896 AA716567 AA934774 H62600 H09497 BF943762 |
| | | BE395335 BE883333 |
| 428342 | 6712_1 | AK056315 AI015524 AA724079 BI713619 AI377728 AW293682 AI928140 AI092404 AI085630 AA731340 BM469629 |
| | | AW968804 |
| | | AA425658 |
| | | AA769094 BF446026 AW118719 AI332765 AW500888 AW576556 AI859571 AW499664 AW614573 AW629495 AW505314 |
| | | W74704 AI356361 |
| | | AI923640 AW070509 AI521500 AL042095 AA609309 AA761319 AI381489 H45700 AA761333 AW265424 AA909524 |
| | | AA635311 AA649040 |
| | | AI392620 Z40708 AI985564 AW263513 AA913892 AI693486 AW263502 AI806164 AW291137 BI061872 BI059498 |
| | | AA134476 AW084888 |
| | | AA036967 AW370823 T55263 BI002756 AA489664 BF827261 W74741 BF963166 |
| 421902 | 276321_1 | BG171436 BE079601 BE079534 AA299964 BE392717 BE883402 BE079532 BE018148 BF889427 W00396 |
| 427043 | 2736_6 | BF511666 BF447624 BF817650 AW974087 BF437901 BE504352 AA568952 AA460669 BE220596 AA397679 AI217920 |
| | | AA399531 |
| 421014 | 24169_1 | NM_012227 Y14391 AK024630 BC014636 BG281479 AL556021 AW778818 BF196455 AW592274 AI376720 |
| | | BI438310 AW511106 AW304443 |
| | | AI568597 BE326797 BG056985 AI569473 AI475975 BF448109 AA853022 AI886592 BG755959 BI084711 BF683496 |
| | | BE177141 AI742415 |
| | | AW001158 BG747230 BI116434 AI984821 AW0816S9 AL543852 T08792 BF526529 AL582296 AA830141 AA789136 |
| | | H26527 AA425013 F32744 |
| | | N62859 AA535316 BE872483 |
| 455187 | 1107511_1 | AW864071 AW864059 AW864056 AW864070 AW864050 AW864067 AW864072 AW864057 AW864065 |
| 459487 | 135353_1 | AA699665 R84889 |
| 435374 | 129988_1 | AI049889 AI733663 AA678730 |
| 425164 | 1276257_1 | BM462532 R18189 R22638 R22639 AA351604 R17880 |
| 416435 | 18099_1 | BF107196 BF804739 AA249165 AA227714 BI002580 BI254772 BE856761 AA843533 AI692783 AI769103 AI479234 |
| | | BF055564 AI332623 |
| | | AI560964 AI431301 BE675257 AA406642 AA707195 AI961161 AI218736 AI149261 AW474460 BE466679 AI989624 |
| | | AA765123 AA180333 |
| | | BF507849 AI281064 AI500253 AW468519 AI473781 AI263097 BF433898 AI912120 AI692780 AI262308 AI266734 |
| | | AA923774 AA227960 |
| | | BE644676 AI034302 AI352401 AI869528 AA862001 AI221573 AI565227 R60069 AW593430 N67562 AI346969 |
| | | AI925794 C21540 BG291961 |
| | | BG255029 BF852350 BF805364 BF852050 BE504626 BF432250 AW947402 AW071704 AW008413 BE717229 H94353 |
| 419560 | 16064_1 | AB033081 AK074324 AW450283 BM128413 BE550945 BE670081 BE674137 AI298087 N30050 BF110807 N30072 |
| | | AI288098 N30068 |
| | | AW861931 AW858575 AI363734 AK025483 BF882045 BM128581 AW771330 BE669540 NM_016376 AB037360 |
| | | AA399607 R94364 BE514643 |
| | | BG684040 BG759578 T83725 AW858561 AW858524 H65293 AW754345 AK025960 BM477612 BE071291 BF087041 |
| | | BG995615 BE071350 |
| | | AI917153 AA436381 BE815318 AK025717 BE857148 AI741138 AW027718 BI789162 AI492263 AI690072 AI420179 |
| | | BI789249 AI637804 |
| | | AW957459 BF222458 AI094328 AW510893 AW295994 AI760430 BE464934 BE220458 AI042022 AI653315 BM128472 |
| | | AW337972 AW515837 |
| | | AI669489 AW664176 AI768730 AW271660 T16381 AI823834 AJ346113 AI242353 BF437578 AI273673 AW513498 |
| | | AI797424 AI674242 AI277236 |
| | | AW731756 AA373089 AW207589 AW467879 AL550999 N30075 BE883669 BF155456 AI423079 R60281 T16974 |
| | | BG676892 BF927070 |
| | | BI962758 BE811485 BE811591 BE811702 BE811600 BE811483 BE811533 BF927061 BG008257 BF873368 |
| | | AL574815 BF946447 H70934 |
| | | BF939868 BF378761 BE082381 BG984527 AA244447 BG983567 AW885571 AW293182 AW885607 AA811057 |
| | | AA610310 AI434406 AA514352 |
| | | H71020 BF740351 BE814815 N66648 AA737686 AI025403 AA255490 N26754 AI094233 AA629548 AA716234 |
| | | BF343632 AW363633 H62663 |
| | | N40757 N55959 AW858569 AW604731 N40773 N40777 T83895 AI905899 BG007487 BF820299 R60231 R60787 AA456130 |
| | | AW383736 BF087045 BG995619 |
| 441673 | 2674292_1 | AI018657 AA952933 |
| 412556 | 1227313_1 | AW961963 T08284 R14658 |
| 454877 | 195659_1 | AW836463 AW836621 AW836541 AW836569 BF948429 AW836577 AW836640 BE072108 BF813547 BE072090 |
| | | BE072048 |
| 400137 | 22730_1 | L42572 NM_006839 D21094 BC002412 D21092 AL581583 BG744690 BE890015 BM145018 |
| | | AW780309 |
| | | AW117715 AW273302 AI935295 |
| | | AI017768 AW117697 AA528074 BM091568 AW026362 AI807072 AI217643 AI754482 BE220790 AU150440 AA488365 |
| | | AU150883 AU149798 |
| | | AW136866 AI037898 AA948432 AL044717 BM091271 AA165119 AI572998 AI159802 AI420707 AA988336 AA465659 |
| | | AA773194 AU146496 |
| | | AA910663 AA584371 AI310749 AI305278 AI951728 AI266446 AI446326 AI283854 AI270681 AI016194 W26444 AI984515 |
| | | AA261848 AI498094 |
| | | AW514651 AA627739 BE887828 AI123491 AA868942 AA677859 AI809730 AI810303 AA496031 AI952462 AA996053 |
| | | AI184087 AA693666 |
| | | AA136578 AA773609 AW900608 AA928404 AW405313 AI907129 AI907123 AI986051 AU150633 AU153325 AU153368 |
| | | AU153017 AI285394 |
| | | AW592096 AI250862 AI869677 AI084968 AA857716 AA862132 AA931138 T96072 H71166 AU151501 AU153688 |
| | | AU150884 AU160131 |
| | | AU152428 AU151809 AU153189 AU152726 AU153115 AU153153 AA191478 AU152952 AA085945 AI092312 AA489369 |
| | | AU144684 R89265 |
| | | AU153572 AW969804 N62534 H04597 BE893641 BE613265 AA136661 AI912911 AA834897 AI886988 AI364744 |
| | | AA813233 H27147 AW874123 |
| | | AI263151 AW732074 AI439378 AI283359 AI203130 BF477675 T10242 AA528336 Z41162 AA502970 AI611989 |
| | | AI802818 AI907127 F03630 |
| | | AA427945 AA928575 AI926261 BE165012 AA188614 AA864217 F25274 AI264826 AI003178 AA115133 AA579080 |
| | | N64601 AA807718 |
| | | AA079255 W37786 H02330 T49457 R49175 H64168 AF148646 BC005983 AU142948 AU131105 BG388364 BM006108 |
| | | BM011315 BG459126 |
| | | AA631333 BI552181 BF220333 AU128630 AU128629 AU129731 AU120428 BI560973 AA075620 AW862160 AW411337 |
| | | AI906984 BE966671 |
| 418733 | 18099_4 | BM473699 N33854 N35083 BE090367 AI306129 AA911920 AA633160 AI302415 AA609954 |
| | | AI365203 AA911919 |
| | | AW995271 AA234587 |
| | | BF996091 AW956344 AW389533 R60133 |
| 400245 | 12188_1 | X79449 BC017853 AL121035 BF196384 AW119044 AI028023 AW451110 AI971911 AW015069 AI079170 AI376367 |
| | | AI264113 AA829646 |
| | | AA737579 AA449679 AA740864 NM_001111 U18121 AL567297 BG773801 BF973874 AV687104 AA527579 AA843525 |
| | | BE706355 AI074589 |
| | | AI523475 BE890249 AW406263 BE074258 AV729485 BF809610 BG058619 AA677244 BE179838 AA622264 AI460106 |
| | | AA740411 AI499168 |
| | | AI078223 AI682923 BE696559 AW375385 AA788739 BG984978 Z40874 T17054 F09669 AW844043 U10439 BI711870 |
| | | AW245957 AU158567 |
| | | AA679305 AA679316 W72510 AI346029 BG059762 AW251062 AA132373 AI925621 AI860230 AI340172 AW192891 |
| | | AI707980 AI094937 |
| | | AI042115 AI200901 BE328452 AA644678 AA551209 BE351065 AA970761 N68609 AW002028 AA160826 AI422774 |
| | | AW873114 AW073597 |
| | | AW664483 AI218710 AW020550 AW190607 AI984545 AI871921 AI333970 AI452887 AI818335 AA398655 AI554424 |
| | | AI274187 BE465703 |
| | | AW512940 AW241366 AI923954 AA576649 AW168294 AA813181 AA912168 AI049738 AW514073 AA548255 AI569630 |
| | | BE710031 AA244182 |
| | | AI341697 AA563904 AI537990 AW517908 AW172943 Z39498 AI750294 AW150414 AI253293 BE825720 T31860 |
| | | AW150775 D20310 AA150892 |
| | | AU133933 BE781148 AL038957 BF910979 AA352297 BG988U2 AW372175 BF229106 AW866705 BE093482 BG990396 |
| | | AI499917 |
| | | AA054452 |
| | | H05484 AI828502 BM467331 AU140570 AL135417 BF947202 AW391926 BE813418 BF998473 T92021 BI021048 |
| | | BM048783 AW501366 |
| | | AW501342 AW501549 BE939021 BE707147 BE160974 BE305207 N49011 AA947119 AA678801 BE536876 AW897428 |
| | | BG329648 BG818540 |
| | | BE542344 BI919250 81253018 AW130996 BE074249 BE895428 BI034862 BE083277 BF952166 |
| 432053 | 11371_6 | AL519914 AL562407 AA778932 BF111318 AW467038 AV729266 AI269598 |
| | | BE939277 AI026690 AW269662 AI024572 |
| | | BF909447 N58843 |
| | | R06838 R07017 T83038 R07054 T90358 N75259 |
| 406782 | 0_0 | AA430373 AA968771 |
| 407644 | 27910_1 | D16815 AL563603 BG399756 AI935028 N41813 L31785 AL529953 AA428473 AA401262 BF802576 |
| | | AA373868 BE886120 BM479352 T28309 |
| | | N57167 BI551531 BG037160 |
| 408731 | 11725_2 | AF305826 BE350971 AI765355 AW172600 AI310231 BM271766 AI547292 AW612019 AI674617 |
| | | AW138666 AI147629 AI147620 BF857810 |
| | | BF886300 BF885952 BF886303 BF885956 BG565497 BE670834 AA114025 BF886396 BF886928 AV750861 D62864 |
| 436395 | 47269_1 | AJ227900 BF512806 F00947 BG614207 BF103731 AA310707 AI094933 BM023235 AW051119 |
| | | BE882892 BG567751 BG567665 BG504662 |
| | | BG562750 |
| 435745 | 37547_1 | AK056461 BG686518 BE387225 AW967059 BE297996 AL135538 BE294837 AW449002 AW451121 |
| | | T16848 AF055029 AL134724 W60898 |
| | | BE856266 AU148963 AW575704 AW152356 AI084072 AW054888 AI935155 AA284068 AW007543 AI986179 AI857721 |
| | | AW190129 AA150977 |
| | | BE208028 BF439037 AA833823 AI377038 BF941216 AI829982 AA468807 AA847169 AI218694 AI129353 AI934898 |
| | | AI184525 AI379211 |
| | | AW769491 AI307200 AI128809 N27928 AA431873 AA136861 AI750062 T03747 AA554767 AA450096 AI278743 |
| | | AA706407 AI039607 AI654580 |
| | | AI031667 AI061299 AA733104 AI359421 AI869544 AI200267 BI335847 BF589867 AW008863 AI471924 AI222650 |
| | | AW662717 AI803848 |
| | | AA887775 AI282703 AI246010 AA721306 AA470395 AI245744 F10546 F09656 N67194 C04809 Z38698 AW580747 |
| | | AA769725 AW374451 |
| | | AW953981 AW578740 BF887002 BE617814 BG685745 AW855788 AW578790 AW578794 BE767704 BF874200 BE767729 |
| | | AA365623 |
| | | AW057997 BE938847 BG572984 BE536806 BI598918 W76362 AA371887 BE703956 AA143026 AW578800 AW578799 |
| | | AW578802 AW578791 |
| | | AW578764 AA193300 AA284124 BF369529 BM458893 AW403361 F12947 AA336566 AA331865 T75183 H00830 |
| | | BM469137 BI256518 |
| | | AA344657 T08539 BG980264 AA873643 BI871195 N40688 H14973 AA337355 AW977829 AL134723 AW452446 |
| | | BE785578 BG977972 |
| | | AA317124 BE962224 BE962220 AW379371 BE935858 |
| 445447 | 6500_2 | BF966635 BF672146 BE180458 AI686634 AA523860 AW271362 AI861821 BF431163 AI022823 |
| | | BF444980 AI765435 AA573781 BI712102 |
| | | AW612498 AI262492 AA398744 AA505593 AW196232 BF941119 AI685545 AW130178 AW044369 BF430926 |
| | | AI275587 AI025611 AI309750 |
| | | AA815111 AI149346 AI174556 AW003736 AI469520 AW992548 AI204623 AI270522 AI084323 AW576339 |
| | | AA922706 AI306116 AI554469 |
| | | AA825775 T03628 BE243372 AA436568 AW263457 AA587932 AA587928 AW262892 R79805 AI681690 AI681693 |
| | | AA393332 BE221894 N50660 |
| | | AA915886 BE702216 H24038 AA074283 R70676 AW576178 H01876 R81147 R81148 R76841 N27519 AW593637 |
| 414405 | 112689_1 | AL047596 AA393792 AI670731 AI037957 AW874364 AI038137 N62286 AI241379 |
| | | BE501096 AW090696 AI927369 AI669226 AI369437 AI371075 |
| | | AW612409 AI686711 AI183289 AA477717 AI076122 AA635190 AA700984 AA781508 D81020 BF575223 AI356183 |
| | | D79312 AI375558 H61111 |
| | | BG283489 BE090666 BE090664 BE090662 H26545 |
| 445317 | 844744_1 | AW468777 AI219856 AI873492 |
| 419439 | 62687_1 | BC007205 BI757428 BG768253 BE793505 BI756175 BG700401 AW247795 BE378793 |
| | | AA485818 BG110861 BM006282 |
| | | AI003793 AI351302 |
| | | D80663 AI457745 AW248460 AA242934 AA456472 AL532788 AA536088 AW119049 AI472227 BF056172 AA827644 |
| | | AI688691 AI590089 |
| | | AI885138 AI885668 AW514618 AI368119 AI312763 AA587359 AI800927 AW629702 AI083644 AI306565 AI569261 |
| | | AI311737 AW514616 |
| | | AI309534 AI167269 AI220241 AI290334 AI352197 AA470701 AW613233 AA252032 AW248068 AA668416 AA252804 |
| | | AI038616 AA909996 |
| | | AA872009 AI272751 AI334419 AA454578 AI688702 AA485686 AA665245 AA878526 AI245998 AI871261 AW058650 |
| | | N31716 AI869672 H20492 |
| | | AA639593 R85327 AI951564 AI954026 BM007182 BI836891 BI758274 BI256473 BG331377 BG679123 AL532835 |
| | | BE393403 AI400134 |
| | | BG742026 BE389332 BI601732 BG698900 AA314318 BM006355 BI002658 BE252908 BG029560 BI196755 AA991163 |
| 423215 | 880395_1 | AW963653 AA323281 AA372970 C16770 |
| 424571 | 9758_1 | BE379766 AW152643 AI803450 AI564343 AI092711 AI140525 AW152156 AI620740 AI554689 AI161209 |
| | | AI290242 AI339745 AI374611 AI347388 |
| | | AI858296 AI140529 AI366124 AA493912 AA406235 AA493889 AI057160 AW022264 AI097277 AI144126 AI080051 |
| | | AA983529 AA860507 |
| | | N53469 AA843767 N81163 N70628 AA424577 AA983537 BF003004 AA626688 AA235977 AI057152 AI095366 AI095356 |
| | | AA458646 AW194479 |
| | | AA150439 AI375272 AW571777 AI359198 AA993793 BE614394 BE738239 AA127883 AI034344 T59504 D81608 |
| | | AA908704 AW051665 |
| | | AA382785 AA307208 N24639 AI370715 BE244980 AA548596 AW449675 AI191008 BF223749 N70752 N22266 AI191012 |
| | | AA028001 AI419106 |
| | | BF215661 BF591548 BG942356 AI474968 BE858217 BF793358 AV756758 BG483603 AI093724 BF693395 BG545345 |
| | | AI744294 T59549 AA811773 BG499757 |
| 422711 | 16701_1 | AK055669 AL049378 AW377544 AI033709 AI472253 BI963224 AI743449 AA621561 AI749846 AW804052 |
| | | AW020579 AI097382 AA016179 |
| | | AI290937 AI016441 AA988531 AI339453 AW470330 AA931074 AA026185 BI491566 AA953819 D80556 AI479362 |
| | | BE856176 BE856995 |
| | | AI418530 AA453318 AA315363 AA974727 AU185136 BE817293 AI825808 AW117512 AA993526 AW517982 AI937289 |
| | | AW005886 BG034928 |
| | | AV716846 BG655435 BI964900 AW371588 AA811110 W31610 C15669 AW161626 AI683982 AA120857 BF222558 |
| | | N39089 AW261816 |
| | | AW167241 AW003602 AA873113 AI633319 AI953905 AA451704 AA287917 AA588470 AA134756 AA872118 AA554169 |
| | | N59186 BG655495 |
| | | AA026186 BF435907 BE049379 AA984507 AA987200 AI803323 BF445688 AW440533 AW439141 AW661740 R76524 |
| | | D60440 BF942226 |
| | | BI850115 AI432094 BF942170 AW262510 AA370302 AA885665 R62142 AI567199 C00263 BI495346 W15536 R37823 |
| | | AW673791 BF989408 |
| | | AA134662 BE300698 R15856 AI625064 AW591350 BM148617 D80991 BE004938 R76240 AV735001 D60083 |
| 444889 | 722791_1 | AI201251 AI917770 AW293546 |
| 418751 | 21393_1 | BF690141 BE159368 AI937311 AU151256 AW341542 AW274231 AW341609 AW450447 |
| | | AA843698 AW188066 AW007171 AW007027 AI075008 |
| | | AL520351 AA608992 AW151842 AA622181 AI273454 AI005661 AA205946 AA677899 AW473512 AW296620 AA872899 |
| | | AI094216 AI025188 |
| | | AA256998 AI539154 AI500192 AI289493 AI040740 BE167841 AW151374 BE167754 BF036108 AA299181 N23237 T62967 |
| | | W96060 AA574412 |
| | | AW606697 W96059 R51303 R09158 R59113 T48473 T59023 AA122066 AW606643 R38386 R06567 |
| 458196 | 60860_1 | AK057790 AU159145 AI802408 BE856709 AI302762 AA907424 BE045678 AI302328 025594 N33153 |
| | | BE326823 AI863332 AV739134 AU149843 |
| | | AI279233 Z20793 AW173558 |
| 430718 | 583102_−3 | AI792814 |
| 419175 | 35068_1 | AB018322 BC012480 BI524873 AW665554 AI934469 AI479916 BF096179 BF096162 BF096132 |
| | | AA744972 AI951988 AI858339 BE076331 |
| | | AA886998 AI570585 AI916688 AI678811 AI693109 AI308135 AA669046 AA961064 AI018062 H80618 BE221942 R52609 |
| | | AI915164 AA365626 |
| | | Z44671 BI052776 BF882486 BG286184 AI589558 AA931663 AA534979 AI275392 AI273455 R52553 AA829920 H80652 |
| | | AA360728 F10618 |
| | | AW953666 AW176773 H85527 AA765570 AA081927 BF093262 BG743753 AL037576 AA534314 BE814964 BE973713 |
| | | N49493 BE006634 |
| | | BE006630 AW270037 AA234765 AI334004 BF057179 AI857450 AI341191 AI434143 AI917449 AW517207 AA255424 |
| | | AW008334 AA847572 |
| | | AA994211 AA861901 AA581873 AI580157 AI364363 AW242357 AW235291 N55645 AA319869 R36911 AA256551 |
| | | AW044188 AI203159 N49403 |
| | | F02090 AI187299 AI609644 Z40516 AW952314 |
| 446436 | 12330_1 | BG474471 AW963784 AL541138 BI091480 AI023457 AV658395 BG991788 BG168902 AL568755 BF515750 |
| | | BG413457 AI073545 AI457986 |
| | | AA553973 AA826823 AI025831 AA722816 AI078414 AI252218 AA570134 AA313183 AI689327 AI609660 BI089971 |
| | | AW614345 BM145877 |
| | | AW182468 AI138446 AW054973 AI433252 BE392690 AA644254 AA621649 AI027916 H74280 H74279 R05903 BG504738 |
| | | BE243125 BG398720 |
| 442961 | 60316_2 | BE966247 BE220885 BE467384 BE350135 BE672094 AI811582 AW665254 AA772731 AI283601 AA417067 AW197746 |
| | | AI868357 AI792143 |
| | | AA931120 AI758506 AA843761 BE737582 AW379586 N38812 BG567321 H13257 |
| 430844 | 450932_1 | BM470823 AA487679 AV736625 T94960 T95013 |
| 434995 | 122184_1 | AW974995 AI791196 AI821880 AI821932 AA659617 AI821137 AA658925 |
| 446096 | 2855992_1 | AI633717 AI276454 AI275116 |
| 451050 | 11847_4 | BM453041 AA760783 BE218582 AI340046 AW166131 BF515854 AI630296 AA461307 AI090881 AW023059 AA155797 |
| | | AA115486 AL597396 |
| | | AW889004 AW937420 AA137082 AA013374 BG619478 BG401839 |
| 443547 | 137089_2 | AV645808 AA701657 AW271273 AI796734 AI472316 AI017531 AI061178 BF109096 AA548964 N83805 AA131648 |
| | | AA156589 BE708349 |
| | | AW952494 D30877 AV684717 Z24837 F00167 BF576150 T63841 R78995 N87474 |
| 449901 | 1269988_2 | BI227033 AI674072 |
| 425996 | 138046_1 | BM006551 AA367152 AW953705 AI631833 AW237429 AW027804 AA729038 BE503409 AI521935 BF739953 |
| | | AA702982 AA557633 AA780065 |
| | | AI218139 AW194264 D20120 AI082715 AI969980 BE857686 BE326711 AW953706 AI393749 AI383821 W67199 AI431759 |
| | | AI796526 |
| | | AI521794 AI796380 AW117545 AI749657 AI537634 N50122 |
| 448797 | 14543_1 | BE614460 BF115119 AI670868 AW590674 AW003998 AI925103 AI521807 AI861967 AI765452 AI758865 AW028509 |
| | | AA708099 AA836286 |
| | | AI468465 AI208587 AW952300 AI824281 AA447219 AA468043 AI373421 AA708115 H13541 BF093497 AA461538 |
| | | BF377943 C01108 BF377920 |
| | | AV714521 AW206741 AW968126 AA774415 AI350171 AI275658 |
| 441969 | 2681178_1 | AI733449 AI733386 AA972780 AA988876 AI791798 AI791748 |
| 441894 | 2338_5 | BE869838 AI148042 AI827471 BG426916 AI684451 AI949551 AW006378 AA535914 BF222911 AI925829 AL044604 |
| | | AW769002 N62372 N52347 |
| | | AW002732 AI537926 AI886148 AA953912 BI862037 D61326 AA055633 BE544632 AW954810 N75119 |
| 433009 | 2142268_1 | AA761668 AA573621 R09670 R92814 |
| 425913 | 4123_2 | AV703649 AW959818 AI582119 AI523459 AI674473 AW663543 AA825476 AI935231 BF742805 AA426156 AI253626 |
| | | AA846477 AI024230 |
| | | BF221780 AI493027 AW006841 AA814699 H99650 AI088977 AA442691 AI783526 BF207915 AI752847 |
| | | AA782635 AI978980 |
| | | AA788634 |
| | | BG119454 BF678528 AI627829 BG993975 AV701283 |
| 443959 | 2780939_1 | AL120546 AI093444 |
| 433359 | 1155535_1 | H70007 AW896257 AW896334 AA584680 AW896386 |
| 435931 | 55043_1 | AK057055 BI868744 AL537388 AL601343 BI917121 AW965676 BE163455 AA401842 BI836992 BE169604 |
| | | AW603254 BF224353 BI000120 |
| | | T09411 T33851 AL566789 AW383995 E504296 BE670066 BE221088 AW612265 A421866 AW044202 BG107771 BE621522 |
| | | AA400384 |
| | | AI668813 AI680704 BE855622 AL047416 AI085944 BE328123 AI951915 AI693159 AI633745 AI472220 AI127849 |
| | | AI601128 BF224203 |
| | | AA403117 AI624409 AW510999 AI199722 AI375679 AI369300 AW008158 BF437759 AI142992 AI299713 AA987655 |
| | | AA954216 AA081613 |
| | | D53173 N90948 N40544 AW172528 N94307 AW274872 AA833541 T03787 AI144167 AI678975 N58937 AI864431 |
| | | AI064741 AW664341 |
| | | BF222277 BE327850 AI625361 AI032746 N73790 W58072 AW204818 W02128 BF445630 AI073621 AA022615 AA084503 |
| | | AI215722 BE856978 |
| | | T56046 AI581993 AI139961 AI569338 AI207177 AI873359 AI682604 AI215528 AI700342 N90316 AA610054 F08873 |
| | | AI459512 AA676678 |
| | | BF589573 AI383046 AA947775 T90475 T90482 T33850 AA009789 W73132 H22902 F10123 H67882 T56062 |
| | | R11653 T89037 N27199 BE169725 |
| | | AA022614 D81394 AA502780 AI093041 T03878 W32393 N99415 AW418560 BE834542 AW197315 AI025774 BF793398 |
| | | AA994729 H50611 BE965743 BI766426 BE536391 |
| 434747 | 117643_1 | AW976537 AI033582 AA837085 AA745261 AA648395 |
| 416727 | 1988165_1 | W05023 N59520 H80405 |
| 400120 | 133_1 | BC014030 NM_004068 BC004996 AK057883 D63475 BM468205 BG386792 BG750447 BG575842 BG479084 |
| | | BG741027 AU118129 BE901043 |
| | | AU141281 BG825395 BG338276 BE396231 AU121493 AU131489 BF341132 BG335659 BI769251 AU142779 AU124483 |
| | | BG480828 AU121353 |
| | | BG702326 AU137866 BG759046 BI460601 BE887290 BG750415 BF127710 BG757819 BI160266 AU122086 BG824046 |
| | | BG744180 BE884943 |
| | | BM006610 BG702599 BI870749 BF307033 BI334771 BG480606 AU142599 AU141336 BM018563 BI335565 AU138308 |
| | | BE391241 BG774488 |
| | | BE278025 BG717959 BG706920 BE312567 AU138300 BE303034 BE303016 AU139252 BE336797 BG470940 BE336735 |
| | | BF305197 AU134288 |
| | | AU139907 BE261245 BE208718 BG761912 BF212890 AW732692 BM011258 AW247629 AU136696 BF530078 BF204146 |
| | | AL048752 BG328927 |
| | | BE388385 BE260122 AU138789 BE253465 BF733914 AW249415 AW239535 BE311791 BE256236 BF529742 BG770465 |
| | | AW245777 AW245813 |
| | | BE296677 BE266852 BE168115 BE396596 BE280057 BE168229 AI750820 AU134137 BF792191 AI272215 AI907348 |
| | | AW238875 BF805152 |
| | | BF568397 BE712727 BE081443 BE001805 BF724536 BF744705 AW247385 BE796369 AU133759 AV705142 BE794402 |
| | | BM465821 BG281284 |
| | | AW384831 BM450689 AU134125 BE311650 BF356318 BM462831 BG420555 BE749127 AU134590 BM019438 BC013796 |
| | | BG761223 BG122058 |
| | | BE872076 BG748496 BG821374 BE619159 BG423244 AU137110 AU127210 BE270081 AA496860 AA351380 AA356303 |
| | | AU099781 AA355912 |
| | | AA371411 AA325535 U36188 AL569574 AL577204 AL525543 AL567342 AL567334 AL567122 AL536527 AL567098 |
| | | BG681585 BG824951 |
| | | BI870652 BI225855 AW393878 BG750632 BI223803 BE877552 BE270473 BE389392 BE779021 BM017845 81117816 |
| | | BE396513 BM049006 |
| | | AW393945 BE786941 BE267724 BE562981 BE314236 BF744102 BM019781 BF806063 BF828673 BF829181 BI008788 |
| | | BF829180 BF829175 |
| | | BF433802 BE909928 BF839784 AW578564 BF943095 AW328030 AA448596 AW882688 AA909846 BF924341 AW361460 |
| | | AA425174 BE940557 |
| | | BF761585 BF931276 BF934886 W68597 BF933977 BI035906 BE836064 BF837576 BI018368 AA205908 H54612 R88902 |
| | | BE812330 BE932300 |
| | | BF924562 AW805376 BE769859 R87381 AA371901 BE838855 AA326381 W56191 AA341231 AA464093 AL575977 |
| | | AL518650 AL547393 |
| | | BG338327 AL517563 AL536921 BG207096 BG207589 BG199290 BG220159 BG744842 BG104730 AA555035 AA618009 |
| | | AA861062 AA610582 |
| | | AW245418 AI040983 AA521380 AW245455 AU147292 AU155236 BE620286 AW250767 BF888236 BE620819 AU154343 |
| | | AU150827 AU148334 |
| | | AW069495 AA701091 AU155225 AW250019 AA666235 AU147764 AA449739 BF907598 AW043731 AU144390 AI924565 |
| | | BE300631 AW874021 |
| | | BE208088 BF732773 BF594057 AI159873 AU143930 N24100 AI052499 AI989370 AI366151 AI139248 AU144776 |
| | | AA838250 AI095433 AI269227 |
| | | BF811358 AU151352 AA477554 AI131290 AI049797 AU157349 AA620559 AA581533 AI338299 AA402755 AI050079 |
| | | AU157614 AI280691 |
| | | AA706590 BE206615 AA861056 AU153214 AU155712 AW069554 AI081124 AU155846 AI081123 AA719876 AI754976 |
| | | AI027763 AA723095 |
| | | BI518782 AW050706 F37521 R79062 AA565530 H39826 AA404670 AU158884 AI139682 AA513633 AI283202 AA651856 |
| | | AI623515 AA427413 |
| | | AW248474 BF841432 BM453246 AI302390 AA808269 AI249357 AA132775 AA716732 AA479932 AI198296 AI208674 |
| | | AI088433 F36977 |
| | | AA496899 AW615762 AA602541 AA341161 D58785 T15399 AI286077 W69152 F28418 AI272156 AA338034 AA568455 |
| | | BE206121 AI080033 |
| | | AI699680 AA861255 AI953465 AI613240 AL581773 AL531577 BF526361 AA657809 AW152670 BE621523 AW078705 |
| | | AW673719 AA102613 |
| | | AA195604 R72209 AA402208 AA404305 N24999 AI963535 D54741 BI461415 BM475959 BI260926 BI116213 BE280120 |
| | | BE940258 BE280044 |
| | | AI905744 BG001474 BE937718 BF799283 BI091621 BG421006 BG289235 BF736825 BI253429 BG170064 R72512 T92815 |
| | | BF931257 |
| | | BM455183 BG033362 BG574220 N31395 BF739185 AA371995 BE879011 BI198754 BF378989 T12266 BM019421 |
| | | BI194570 BF378992 |
| | | AW996595 BI858649 W94605 W61345 AA122384 AA171923 T92736 AA761504 AI819039 AI439358 AL517777 AL565919 |
| | | AA622001 AA642695 |
| | | AA704144 AA081465 AA070621 AA375562 AA700011 AA926863 W37310 AL566236 BF677809 BG760021 AW361433 |
| | | BE828605 BE268449 |
| | | BF805977 BG292452 BF981071 BF217108 BF928698 R33993 AW882841 AI857453 AW078733 AI433035 AI018103 |
| | | R90927 AA804720 |
| | | AA551734 R33835 H19741 R78754 BF930494 BG109583 AA631926 BE834008 BG996533 |
| 410972 | 480035_1 | BG113822 AW812262 AW812259 AW812255 |
| 438021 | 45849_1 | AY043361 NM_007013 AL136739 U96113 AL049041 AI750898 AA054855 BF887778 BF333173 |
| | | BI061338 BC015380 BG569062 BM090890 |
| | | AI769300 BE466554 AI091728 AI800660 AA832035 AI342285 AI750899 H66486 AA455942 H20821 AI187877 AI417440 |
| | | AW198099 AI245158 |
| | | T91227 AI373068 AW194829 AA680229 BG291204 AW885601 BF380168 D82380 D82098 BF885552 AL050082 BE552267 |
| | | AI458088 |
| | | AW137654 BM014715 AA418921 AI159868 AA845398 AI743920 AI159888 AI653531 AA843952 AI799752 AI360957 |
| | | R67562 BF445478 |
| | | AA854844 AA552895 C21152 BE614563 BI260650 AU155187 AW169673 AI915801 N29619 AA435618 AI361252 AI424627 |
| | | AI219018 R67514 |
| | | AA548141 AI246669 AA156867 R67561 R67513 AW263860 AA089478 AV653790 AA354846 BI560015 AW794547 R52020 |
| | | H18923 R67555 |
| | | AI302490 AL582101 AI871884 AW029104 AI582326 AV688960 AL040820 AI804339 T57931 R67554 AA418232 |
| | | AA393281 BE076206 |
| | | AW189435 AW979344 AW953494 BF056797 AA310720 N64062 W92065 AI761704 AA418824 H89156 H88930 W92048 |
| | | AW33096 AW007773 |
| | | AI745541 BE504125 AW243111 AI201009 AI187909 R44411 AI795988 BF222383 R29593 AA156952 |
| | | BF029856 |
| 411227 | 1073598_1 | AW833298 AW833294 AW833272 AW833271 AW833274 AW833308 AW833275 AW833296 |
| | | AW833295 AW833258 AW833306 |
| 442751 | 1767112_1 | BE567123 AI358404 |
| 446164 | 41648_2 | AK026817 AI559708 AW273539 AW892986 |
| 450377 | 12109_1 | AB033091 AL520743 BE811813 N53332 N99716 AL561910 AA280655 BE710392 AV705100 AW293978 AW444556 |
| | | AA281459 AI679751 |
| | | AI873695 BG700891 BI553517 R80518 BG779771 BG534451 AA479402 AW961580 BF061430 AI857643 AI768486 |
| | | AW512118 AA479302 |
| | | AW770384 AW072470 AI041596 AI049699 AW592865 AA976261 BE879747 BG114119 AW183811 AA909938 BF571621 |
| | | BF350794 BF351375 |
| | | BE925699 AL050294 BC010371 BF982270 AL042656 BF095732 AW812618 BF095731 BG212397 BF678765 BI038602 |
| | | BG388664 AW675337 |
| | | BG289398 BE939598 AW805570 AA527097 AW150540 AI693720 AA743364 AI915793 N48185 AI573107 AA043474 |
| | | AI351615 AI969490 |
| | | AI910763 R50866 AI699181 N73808 H08164 |
| 433991 | 2208777_1 | AA824302 AI806816 AA621207 AA621208 AA912749 |
| 459169 | 3146528_1 | AI905455 AI905452 AI905517 |
| 441458 | 306062_1 | AW815108 BF928501 AW815254 AW815139 BE064292 AA935099 AA934531 BF955469 BI009962 |
| 421272 | 64950_1 | BG819964 R87567 BF062036 BF062044 AW014470 BG057316 AI810506 BE466071 R88291 AA704157 C15898 |
| | | C15173 AA286825 BF594141 |
| | | R84605 H85515 |
| 400200 | 3806_1 | BC005265 BG176720 AW006027 BM352064 AW026316 AI635822 AI880584 AI693769 AI092211 BI492387 |
| | | AI400449 AW166297 BF939910 |
| | | AA232282 AW021432 AI333893 AA494308 AA854899 AI436795 AW069256 AA682373 AI092748 AA993184 AI126077 |
| | | AI081758 AI240686 |
| | | AI261863 AI378423 AA465237 AI376096 AA035579 AI087306 AA448162 AA129977 AI090903 AI080686 AI288939 |
| | | N33004 AI801240 |
| | | AW021546 |
| | | AI370773 AI086064 AA669528 AI250053 AI870113 AA853181 AA858014 BG055562 BG939559 AW080765 AA032283 |
| | | AW467587 H40506 |
| | | D00762 NM_002788 AA641134 AI582295 AI417525 AI563975 AI093566 AI707743 AI290741 AW073417 |
| | | BE875418 BM264076 BG876884 |
| | | AI680535 AW854219 BE774635 AW854212 BG952443 AW854221 AW854208 BE156348 BE843056 AW858991 |
| | | BE937569 BG878291 |
| | | BG876450 AW819099 AI908570 AA449871 AU135228 BM478404 BF126296 AA375499 AA248473 |
| 425261 | MH337_5 | AK054563 AF339812 AA528225 AI819825 BF434272 AW009758 BM023283 AI367158 AI954564 AA564676 BM353611 |
| | | AI151187 AW518909 |
| | | AI688072 BE207868 AA680236 AW001919 BE206150 AI814780 AW513625 AI204573 AW517297 AI276223 N49193 |
| | | AI868635 AA877533 |
| | | AW514291 T03476 M410444 AA973195 AI826725 AA662719 AW071134 AI620613 AI285995 AI241782 AI243244 |
| | | AI344384 AI017846 AI423377 |
| | | AI346069 AI399834 AI366843 AA814351 F25816 AA927979 AI141159 AA720769 AA953724 AI383799 |
| | | AI572402 AW002178 N29888 AW472873 |
| | | AW193742 AA614853 AI476008 AW771672 AW083617 AW591173 BM450662 AI922272 BM463462 BE879542 AA149308 |
| | | BG205668 R94706 |
| | | T98497 BF917910 BE964015 AI311930 BF303765 F22493 D20169 AA147668 |
| 435882 | 136881_1 | AA701431 AW194987 AA704261 AA705688 |
| 450456 | 10568_3 | BI869374 AW608173 AA481283 AA779091 AI091407 AI380767 AI161247 AA775262 AI804165 AA742296 AI620960 |
| | | BF057560 BE644921 |
| | | AI360753 AW271412 AA243424 AI082407 AA779083 AA670202 AA233657 AI032843 AW768251 AW016884 AW838625 |
| | | AA233693 AA766578 |
| | | AI246454 AI097182 AA774648 AA630743 H25879 AA233157 BF475907 AU153405 AI342173 T50088 N23013 AI817953 |
| | | W15432 AA404548 |
| | | AA009914 AW014445 AA243486 AA291931 N33512 BG987649 BF981538 BG619175 BG977894 |
| 450122 | 1398_1 | BI830320 H45858 BI821969 AA834581 AA999871 BI831412 BI827162 BI826049 AA531076 AA327326 BF344394 |
| | | BI860338 BE382517 BI600376 |
| | | BI598227 BG754268 BI962938 BE907670 AI339466 BE222828 BE906305 AA662096 AI570621 AI088762 AI817835 |
| | | AI310480 BF725056 |
| | | AI669307 AI076748 BI966768 AI310414 BE158972 AW444735 AI333025 AI310004 BE393365 BF940054 BE858296 |
| | | AI498530 AI376399 |
| | | AI138476 AI289161 AI378371 BE857636 N40684 AI301684 AI370578 AI928410 AI568228 AI214670 BE782530 F19317 |
| | | AA468593 AI359175 |
| | | AI300708 BE070822 AA369484 AW959944 AA371588 BI604202 AI308854 AA636048 AA729907 BE743989 W42852 |
| | | AA644403 AA047301 |
| | | AA737174 BG054990 BF338479 AA948684 AA807788 AA844504 AI885673 AI927015 M662133 N98291 AA401721 |
| | | AA045902 T58832 W42908 BG996868 BI521617 |
| 445530 | 8558_4 | BE294791 AA115300 BE677858 AW592684 BF061022 AI969919 H10240 AW183605 AI620788 F13488 AW468877 |
| | | BE172330 AA115340 |
| | | AV654146 D79658 T75088 T75087 R14638 D63008 BG623753 BG926180 R91348 AW885760 AA249136 |
| | | BG926100 BE175717 BE175718 C16690 Z42755 |
| 406815 | 0_0 | AA833930 AI684135 |
| 457393 | 5773_1 | AW970603 AA503455 M85475 AA503437 |
| 451436 | 5742_10 | AA017575 R56724 BE836659 BE836660 BE817256 BE817335 AA258389 AA258459 BE817739 BE817702 BE817325 |
| | | BE836614 |
| | | BE139090 |
| | | AW861931 AW858575 AI363734 AK025483 BF882045 BM128581 AW771330 BE669540 NM_016376 |
| | | AB037360 AA399607 R94364 BE514643 |
| | | BG684040 BG759578 T83725 AW858561 AW858524 H65293 AW754345 AK025960 BM477612 BE071291 BF087041 |
| | | BG995615 BE071350 |
| | | AI917153 AA436381 BE815318 AK025717 BE857148 AI741138 AW027718 BI789162 AI492263 AI690072 |
| | | AI420179 BI789249 AI637804 |
| | | AW957459 BF222458 AI094328 AW510893 AW295994 AI760430 BE464934 BE220458 AI042022 AI653315 |
| | | BM128472 AW337972 AW515837 |
| | | AI669489 AW664176 AI768730 AW271660 T16381 AI823834 AJ346113 AI242353 BF437578 AI273673 AW513498 |
| | | AI797424 AI674242 AI277236 |
| | | AW731756 AA373089 AW207589 AW467879 AL550999 N30075 BE883669 BF155456 AI423079 R60281 T16974 |
| | | BG676892 BF927070 |
| | | BI962758 BE811485 BE811591 BE811702 BE811600 BE811483 BE811533 BF927061 BG008257 BF873368 AL574815 |
| | | BF946447 H70934 |
| | | BF939868 BF378761 BE082381 BG984527 AA244447 BG983567 AW885571 AW293182 AW885607 AA811057 AA610310 |
| | | AI434406 AA514352 |
| | | H71020 BF740351 BE814815 N66648 AA737686 AI025403 AA255490 N26754 AI094233 AA629548 AA716234 BF343632 |
| | | AW363633 H62663 |
| | | N40757 N55959 AW858569 AW604731 N40773 N40777 T83895 AI905899 BG007487 BF820299 R60231 R60787 AA456130 |
| | | AW383736 |
| | | H05232 R59374 H12223 AI935759 AI362553 D60006 N29572 AI916833 N75273 AA148710 BI597117 BG740471 |
| | | AA332671 AA333874 |
| 410365 | 60627_2 | AL050199 AI872323 BE883872 AA424083 BG109354 AI243745 AA834941 AW102813 AW196535 AW973796 |
| | | AA559860 AI475291 AA559908 |
| | | R34681 H96211 Z39807 BF954386 |
| 417628 | 2137209_1 | T84214 Z43709 R05654 |
| 434809 | 118540_1 | AW974687 AA652145 AA649656 |
| 420985 | 455_7 | X94703 NM_004249 AL521205 BF886672 N46403 T87420 Z36855 BF934259 BI549507 BG899191 BI517479 |
| | | BI818431 H39666 |
| 428104 | 1376802_1 | AI205993 AA421350 AA421440 |
| 430933 | 605836_1 | BG284105 BE080356 BF372738 BF372697 AA489179 AW863789 BE179901 BE179402 AW863635 BG995706 BE010747 |
| 421297 | 16064_1 | AB033081 AK074324 AW450283 BM128413 BE550945 BE670081 BE674137 AI298087 N30050 BF110807 N30072 |
| | | AI288098 N30068 |
| | | AW861931 AW858575 AI363734 AK025483 BF882045 BM128581 AW771330 BE669540 NM_016376 AB037360 |
| | | AA399607 R94364 BE514643 |
| | | BG684040 BG759578 T83725 AW858561 AW858524 H65293 AW754345 AK025960 BM477612 BE071291 BF087041 |
| | | BG995615 BE071350 |
| | | AI917153 AA436381 BE815318 AK025717 BE857148 AI741138 AW027718 BI789162 AI492263 AI690072 AI420179 |
| | | BI789249 AI637804 |
| | | AW957459 BF222458 AI094328 AW510893 AW295994 AI760430 BE464934 BE220458 AI042022 AI653315 BM128472 |
| | | AW337972 AW515837 |
| | | AI669489 AW664176 AI768730 AW271660 T16381 AI823834 AJ346113 AI242353 BF437578 AI273673 AW513498 |
| | | AI797424 AI674242 AI277236 |
| | | AW731756 AA373089 AW207589 AW467879 AL550999 N30075 BE883669 BF155456 AI423079 R60281 T16974 |
| | | BG676892 BF927070 |
| | | BI962758 BE811485 BE811591 BE811702 BE811600 BE811483 BE811533 BF927061 BG008257 BF873368 AL574815 |
| | | BF946447 H70934 |
| | | BF939868 BF378761 BE082381 BG984527 AA244447 BG983567 AW885571 AW293182 AW885607 AA811057 AA610310 |
| | | AI434406 AA514352 |
| | | H71020 BF740351 BE814815 N66648 AA737686 AI025403 AA255490 N26754 AI094233 AA629548 AA716234 |
| | | BF343632 AW363633 H62663 |
| | | N40757 N55959 AW858569 AW604731 N40773 N40777 T83895 AI905899 BG007487 BF820299 R60231 R60787 |
| | | AA456130 AW383736 |
| | | BF087045 BG995619 |
| 410365 | 60627_2 | AL050199 AI872323 BE883872 AA424083 BG109354 AI243745 AA834941 AW102813 AW196535 AW973796 |
| | | AA559860 AI475291 AA559908 |
| | | BE670565 AI919258 BI002664 AA557197 AI581597 AW604533 W69379 N66899 H84695 N53879 AA644631 H24627 |
| 455631 | 1488654_1 | BE063040 BE063006 BE063031 BE063002 BE063072 BE063024 BE063008 |
| 432523 | 1031284_1 | AW972205 AA551756 AW815357 AI023998 AW895029 AW856039 AW753791 |
| 440328 | 15103_5 | BG203047 BG208248 BG211369 AW392243 AW392234 AW392226 AW392246 |
| 441229 | 591349_1 | AW083064 AA923345 AI744267 AW517768 |
| 428023 | MH1660_24 | AK027255 BE856281 AW182584 AW027872 AI819831 AI936994 AI278611 AW338658 N29885 AA687514 |
| | | AI824306 AI038904 AI034339 |
| | | AI292064 AA973469 AI291616 AI079733 AI038683 AA491599 AA776023 BE813865 AA993675 AA837380 BF806738 |
| | | BF803246 AI087090 |
| | | BE930098 AA911116 AW137988 AI989393 AI283557 AW175908 |
| 424967 | 2139_1 | BI116740 BM312146 H84589 BI831716 AW468329 W35384 W37216 BG818478 AI188435 BM311546 AI885302 BF448603 |
| | | AI870012 AI797830 |
| | | AI185731 AI336328 AI292299 AI240858 AW057899 AI970695 AI350388 AI292131 AA846185 AW665469 AW002306 |
| | | AI144304 AI306407 |
| | | AA465170 AI283059 AI026124 AA771949 AI038429 AA725179 AA846193 BE773021 AI201261 AI797645 BF593616 |
| | | AI245212 AI301420 |
| | | BE466901 AI203386 AW592346 AW748053 AA854554 BE719868 BG475523 W33173 BI019056 BI831085 BG005729 |
| | | AW075722 AI201542 |
| 415023 | 37164_1 | BG763540 BI115604 BG325282 AY007096 BE048206 BE048060 AA356949 BG469451 BE267760 AI683752 |
| | | AI702004 BE247172 T47611 |
| | | T47612 BE780300 AA932146 AA652369 AI784281 BM055020 BM023299 AI382709 AA159351 BI262171 |
| | | AI863355 AI674922 AI754389 |
| | | AW295190 BE673083 AW469497 AI949042 AW131720 AI990060 AI623178 BE673420 AI863364 AA620354 BF431793 |
| | | AI056058 AI056059 |
| | | BI258269 BI823911 BG482172 BI261556 BI519838 |
| 437306 | 708160_1 | AW977693 AW270552 AA748487 AA767827 AA827207 AA934962 |
| 452479 | 3144985_1 | AI903527 AI903576 AI903573 AI903529 AI903528 AI903525 |
| 431675 | 135623_1 | AA699965 AA513572 AI696381 |
| 432600 | 114582_1 | AW973464 AI821831 AI821085 AA640756 AA657438 AA554802 AA650339 |
| 400271 | 893_1 | BM467047 BI668430 AV723510 AA383564 BM466286 BI756033 BI752301 BE741302 AA312107 BF219348 |
| | | BF224233 AA372905 BE972765 |
| | | BG530510 BG430596 BG430439 D56385 BE566653 AI761953 AI361096 AW771495 T65962 BI494838 AW271309 AI423700 |
| | | AI762323 N72193 |
| | | AW007571 BC013873 X72964 NM_004344 BC008415 BC005334 AL554338 AL559625 AL559603 BG696707 AV733516 |
| | | BI670087 BI600577 |
| | | BI043582 BE866235 BE293715 AI681691 AA283060 AW612235 T29090 BE694176 Z30203 AA379970 W21362 BG179242 |
| | | AI681294 AI694310 |
| | | AI670881 AI074606 BF940039 AI858065 AI681393 AI991986 AI400174 AI910439 AI089029 BE048642 AW614075 |
| | | AW262850 AI431584 |
| | | BI495202 AA582224 BI495201 BF197587 AA459997 AA282970 AI769114 AA632011 AI091029 AI375169 AI400216 |
| | | AA782032 AW044700 |
| | | AI633913 AI129124 AI624690 AI799764 AA916363 BE503032 M492546 AA987867 AI373064 AW772272 AW015455 |
| | | AI368642 AW243721 |
| | | D45517 AI868673 C20558 AI130836 AI928680 AI191010 AA834469 AI963630 N93012 N67416 AL577079 BG532126 |
| | | AI640172 AI670873 |
| | | AI457787 AA460091 BE090147 AA614862 BE182933 AV733272 AI909822 BI494839 BG121897 BE293433 BI044033 |
| | | BF910106 |
| 418876 | 121279_1 | AA740616 AA654854 AA229923 |
| 414783 | 262554_1 | BE972667 AW069569 AI392889 H85161 AI074962 AA423935 BE671254 AA912627 H39845 H69083 AA280259 |
| | | N67746 AA155722 |
| 446841 | 127420_1 | AW958927 AW002054 R00045 AA384616 AA669984 AI571663 BF439133 AA302636 AV660567 R02526 T90529 |
| | | AI678912 AI185622 AI963670 |
| 453929 | 97058_1 | AA045461 AA284145 AI494193 AI469576 AV700262 BF222563 AW190054 AI263834 AA040074 AA045137 |
| 459436 | 880261_1 | AI908449 AA323121 |
| 427224 | 25669_1 | BC014914 AW025233 BG433378 AI348364 AA917746 AL135554 AA708134 AI095765 AI291861 |
| | | AI264632 AI032834 |
| | | BM464145 AW575013 |
| | | AI383072 AA815134 AA765181 D59880 H29890 AA825534 AI696290 M399405 BE669700 BF224225 AI914181 AI674625 |
| | | AI125811 AI022371 |
| | | N63064 R93084 AW977125 BE568027 BG434014 AA403200 AF062366 AW891180 AA401207 AA477849 |
| | | BE764638 BE764629 AW891194 AW439069 AW891199 |
| 442484 | 3699_10 | AI110863 AF075362 AI110861 AF075360 |
| 418831 | 245538_1 | AA488082 AA228970 AA230043 |
| 448079 | 74377_1 | AK054894 AW978550 AI984051 AW205843 AW205839 AI379941 AI383948 AW129532 BF197455 AI954133 |
| | | AI668869 AI360826 AW614392 |
| | | AI619505 AA927599 R64287 AI803003 AI675123 AW023079 AI638324 AW134959 AA831069 BE772132 F29901 |
| | | BF210719 AI376624 |
| | | AW960171 AI275045 BE856533 AW243724 AI051487 R68631 C17731 Z44230 AA309094 BI837972 AA122297 BE929614 |
| | | BE006483 AA098802 |
| | | BG252168 BG928816 BI091300 AW963375 AA213871 BE925757 T90131 BG426009 BG611801 BE090425 BF672863 |
| | | BF132610 BF184312 |
| 450869 | 2827_1 | AK074246 AB062418 AI040384 AA694057 BG566618 R83450 AA011459 H57230 H93947 AA033972 N91333 |
| | | AI040705 AA705512 H57154 |
| | | R05747 H66758 T97233 H61474 AA033971 |
| 437406 | 5564_1 | AL355708 AL390182 AW961056 AW026094 AI201896 AI339096 AW753013 AW377575 AL359597 AA458509 |
| | | AA101706 AA430395 BE218599 |
| | | AI580153 AI337252 AW672987 AW117483 BF477914 AI159869 AI469834 BE328727 AI800118 AI049995 AI739268 |
| | | W40584 AI695716 |
| | | AW008980 AI689764 AI393298 AW139228 AI097595 AI762231 AA454591 BE219638 AI291020 BG149193 AI769982 |
| | | AW189752 AI418017 |
| | | AI363808 AI926110 AW590852 AI524293 AI424269 D30993 AI669931 AW873757 AA355695 AI392826 AU147571 |
| | | AU154893 AI685419 |
| | | BE326844 AW024382 AI271608 BE856703 AI739564 AI588871 BF437288 D55501 AW883430 AI991167 |
| | | AI681404 AA534707 W78925 AI547249 |
| | | W85910 AA702573 AA677590 AA971286 AA452238 AA582421 AA262979 AI023007 AI038705 AA977918 AA454590 |
| | | AA973696 AI091861 |
| | | N66679 R39883 AA868049 AA846818 AA150827 AA151064 AA101707 N50264 N49212 H05824 BF856686 T26533 |
| | | AI983044 AA730190 |
| | | BE856310 AA009812 N20978 AA009813 T26532 T07386 AI084242 Z38902 AA904453 T78860 H84638 |
| 458172 | 363900_1 | BE007237 BE546311 AA984819 BI256810 W19919 BE007263 BG000322 BF327011 AA890198 BE007496 |
| 414992 | 1785181_1 | D63302 D78793 C17962 |
| 410215 | 15479_2 | BI914830 BG209254 AI694353 AI312853 BF593037 AI088624 AW340577 AW015288 BF589940 BF222096 |
| | | BF431760 AA781252 AA449828 |
| | | BF436265 AI572104 AI695710 AI627770 AI524498 AW662141 AI572181 AA988863 AA782797 BI766911 |
| 411115 | 1070123_1 | AW818312 AW818453 AW818380 AW818207 AW818214 BF375509 AW862010 |
| 407604 | 43771_1 | AK074129 AL138071 AI806793 AI453544 AI084616 BF062435 BE672932 AI828296 BF062464 AI803443 |
| | | AI264326 AI280956 AI743547 |
| | | AW295227 AW139967 AI972561 AI863570 AW062752 AW062756 AW062753 AW176904 AW062755 AW062750 |
| | | AW062749 AW176886 |
| | | AI751694 BG251391 AA043621 W30872 BI524827 N94581 AI084614 AA776976 AI377572 AI751695 AI016576 |
| | | AW439994 AW194255 |
| | | AW627763 AA668249 AI079870 AW022524 AI968540 AI248360 AA702490 AA043262 AA631511 AW176883 AW751767 |
| | | AW176887 AW062748 |
| | | AW062745 AW176862 AW176815 AW176814 AW176846 AW176841 AW176871 AW176874 AW176819 |
| | | AI927437 AW191962 R36548 AA853713 |
| | | AW196059 AA618567 AW176881 AW062754 AW062751 AW062746 AW176888 AW751772 AW176859 |
| | | AW751765 AW751762 AW176833 |
| | | AW176873 AW751745 AA694513 AA780815 AW176857 AW176834 AW176839 AW751771 AW176878 |
| | | AW176843 AW176842 AW751755 |
| | | AW176847 AW176812 AW176830 AW176849 AW176844 AW176877 AW751757 AW176850 AW176851 |
| | | AW751758 AW751746 AW176858 |
| | | AW176853 AW751766 AW176845 AW176872 AW176840 AW176876 AW176852 AW751754 AW176836 |
| | | AW176856 AW751773 AW751760 |
| | | AW751751 AL138070 AA639738 AL602588 AA853712 AA317570 R38469 AW291569 |
| 452780 | 73430_3 | BF576403 N79410 AA028160 BE171598 BF090519 |
| 458311 | 2694866_1 | AF069480 AF069479 AF069478 |
| 413533 | 12274_1 | AK055111 BI560412 BI600640 BI549250 BI463611 BG898612 BE468008 |
| | | BG675527 BE146768 BF853416 BE146973 BE146972 BE146767 |
| | | BE146985 BE147021 BE147018 BE146793 BE146783 BE147020 BE146760 BE146954 BE146771 BE147044 |
| | | BE146766 BE146952 BE146776 |
| | | BE147048 AI686856 BE147042 BE147019 BE147025 BE146797 BE146781 BE147030 BG420528 AW294469 |
| | | AW248308 D29070 BG152458 AA908606 |
| 446501 | 2873469_1 | AI308174 AI307311 AI302616 |
| 450371 | 1627928_1 | AI694038 BE464839 AI803494 BE241794 H86709 R34926 BE241681 |
| 414496 | 1526_1 | AK058006 BF724822 W65303 AW887764 AW023806 Z25353 AW022095 AA730973 W00417 W73819 BF982096 |
| | | AI927669 AW188021 |
| | | AW770478 AI913512 AA604358 AI697341 AI691028 AI338392 AI079403 H97538 AI144448 AI253102 AI051402 AI335900 |
| | | AI868132 N28900 |
| | | H98465 AF268386 AI799915 AI819228 BE048413 AW304723 AI819923 BF223106 AA155907 AW298079 |
| | | BF055272 BF446804 BF197697 |
| | | W58588 BF197538 AA032180 AA992597 AW590254 AA027824 AI129369 AI131331 AI655843 AA932907 AW104493 |
| | | AI150615 BF110226 |
| | | AW172271 AI312659 M057312 BE673669 AA722984 AW104985 AI129232 AI078648 AI653086 AI703481 AW515897 |
| | | AI352206 N67076 |
| | | AW297281 AI686162 AA029184 AI610743 AW772016 AI091778 W65401 AI687374 AI218085 AI765158 AI018002 |
| | | AI653068 AI335704 AI520850 |
| | | AW275228 AW275204 AI420247 AA975336 AI697042 AW182235 AA736386 AI281682 AW169698 AW263325 BE645834 |
| | | AI377438 AI146706 |
| | | AA613808 AA716538 BI496247 AA032248 AI698930 AI193399 N70026 H86792 AA404489 W61267 BF447230 AA910805 |
| | | AA150774 AA621907 |
| | | AA902526 AI827634 AW022037 BF059000 BI496246 BG577007 BG571077 AA460779 AW816890 AW816893 AW816891 |
| | | AA029183 AA010295 |
| | | H86850 T83320 BE160823 H12925 N40087 AA096372 BE160847 AW816892 AW816889 AW816882 AW816868 AW816941 |
| | | AW816578 |
| | | AW816940 AW816577 AI431628 AI828113 AA033677 AA033654 AA452704 AA317582 AA346971 BF836584 H48669 |
| | | BI861605 BG925200 |
| | | AA463277 H89048 AA155952 W03252 W01510 W00915 W58589 AA164519 N24017 N24622 N27149 N70109 R43771 |
| | | AA010296 W84611 |
| | | H98889 H88965 AW594424 AA034139 AA065223 N99696 BG981481 N94371 AA767970 W47146 N70977 H05510 W61268 |
| | | T90796 AA164518 |
| | | W47244 AA150883 AA034138 BF338483 F13671 H51317 W72716 N21488 AI188071 AI370541 AI754442 AA148524 |
| | | AI749182 W95221 W92522 |
| | | R20385 |
| 455100 | 1096250_1 | BE160198 AW935930 AW856073 AW861034 AW935898 T11520 |
| 411806 | 1107840_1 | AW864356 AW864547 AW864277 AW864496 |
| 432676 | 3503_22 | AI187366 AA618478 AA558869 |
| 422642 | 873508_1 | AW961201 AA314041 D63247 C18669 |
| 432377 | 1620366_1 | AA535140 AA534670 AA535021 |
| 445298 | 724820_1 | AW295700 AI219199 |
| 446868 | 15525_1 | AK074473 BC017997 BI831060 BF971101 AI886394 AI082824 AV708785 W86073 W07772 AV660737 AI816793 |
| | | R52250 BG183529 AA633473 |
| | | AI191256 R44763 R19947 BF571346 W86257 |
| 425199 | 1276656_1 | R97184 H47493 AA352099 |
| 437714 | 845454_1 | AW977404 AA836116 AA766346 AA809877 AW469598 |
| 426143 | 3806_1 | BC005265 BG176720 AW006027 BM352064 AW026316 AI635822 AI880584 AI693769 AI092211 BI492387 |
| | | AI400449 AW166297 BF939910 |
| | | AA232282 AW021432 AI333893 AA494308 AA854899 AI436795 AW069256 AA682373 AI092748 AA993184 AI126077 |
| | | AI081758 AI240686 |
| | | AI261863 AI378423 AA465237 AI376096 AA035579 AI087306 AA448162 AA129977 AI090903 AI080686 AI288939 |
| | | N33004 AI801240 AW021546 |
| | | AI370773 AI086064 AA669528 AI250053 AI870113 AA853181 AA858014 BG055562 BG939559 AW080765 AA032283 |
| | | AW467587 H40506 |
| | | D00762 NM_002788 AA641134 AI582295 AI417525 AI563975 AI093566 AI707743 AI290741 AW073417 |
| | | BE875418 BM264076 BG876884 |
| | | AI680535 AW854219 BE774635 AW854212 BG952443 AW854221 AW854208 BE156348 BE843056 AW858991 BE937569 |
| | | BG878291 |
| | | BG876450 AW819099 AI908570 AA449871 AU135228 BM478404 BF126296 AA375499 AA248473 |
| 410388 | 36281_5 | AW386165 AA831460 AA772198 AA687734 AI469486 Z39019 AI829314 AI809944 AI969600 BE348981 AW439586 |
| | | AI207192 AI307374 AA084590 AA738148 AA309799 H89072 |
| 442107 | 2684347_1 | AI912225 AI692286 AA976601 |
| 452299 | 89203_1 | BE221822 AA025352 H43596 AI452797 AW206330 BF824812 BF748930 AA025363 BF856465 |
| 440248 | 2616908_1 | AA876138 AI239602 AI698953 |
| 433023 | 3970_8 | BE999967 BF438599 AW864793 AI802899 BE815132 AW468888 AI672189 AI052004 BF112024 AA772335 AW275054 |
| | | AA573845 AI144148 |
| | | AI968683 AA846676 AA927355 H80424 AW973295 R88209 F29868 BE928871 |
| 453085 | 10017_1 | BC017336 BG716430 BG501286 BI458528 AI582223 N98532 AI338138 AI273442 AW102617 AA831177 |
| | | AA745642 AA412583 AA355375 |
| | | BG547492 AW954243 BI766546 BG057641 AI192435 AI338935 AI312651 AI708679 AI191125 AI206832 AA676899 |
| | | AI078010 AI888718 |
| | | AA452830 BF445542 N69930 AA715017 BF446713 BE046852 AW771909 AA907729 AI143749 AI761290 AA890233 |
| | | BF925759 AI783713 |
| | | AI767267 AA814538 W56778 AA918481 BG743526 BE645242 AI025328 AI298436 AI290445 H27710 AI475034 BG740023 |
| | | AI090348 AI340003 |
| | | BI602481 W38495 AI183314 AI927418 BG397181 AA878310 W19369 W56507 C05751 AW380760 AW380770 AW380790 |
| | | BF930729 H28425 AA037326 AA375805 |
| 432541 | 625908_1 | AW973316 AA552525 AW131368 AA614350 |
| 409193 | 22239_1 | BC014317 BC015099 AF229179 NM_020665 BI760941 BG427247 BG429705 BG432624 BG399046 |
| | | BG430790 BF940314 AW242855 AI793025 |
| | | BE048959 BF589928 AA999850 AI767568 AW612416 AI911520 AI793193 AI373739 AI985237 AI433883 AW779364 |
| | | AI253234 AI478325 |
| | | AI524824 AI613056 AI650909 AW299600 BE465778 BF431418 AI470468 AA345162 AI671437 AI473091 BE501448 |
| | | AI431850 BG400513 |
| | | BG429618 BG400845 BG429174 BG429011 BG400319 BG399402 BG427839 BG433974 BG428217 BG426067 BG428990 |
| | | BI762437 BG209271 |
| | | AV653898 BG188580 BG400436 AI765078 BG184374 BG193796 BG431368 AA131483 AA065156 AA076448 BF031348 |
| 458140 | 1648556_1 | BE559806 R12767 BE268806 |
| 445208 | 2826769_1 | AI681579 AI216103 AI692734 |
| 458799 | 643053_1 | AW173151 AI688547 AI768659 AI476487 AI797300 |
| 420820 | 26124_3 | W73822 AI686053 T10508 AA722451 BF750857 BI858823 BF964200 AL577848 W26760 |
| | | W73854 AA476662 AA280705 W26998 W27144 |
| 426532 | 1297511_1 | AA486726 AA380886 AA486725 |
| 434072 | 73667_1 | AF116655 AI114499 BF760333 H70853 BF326768 H70854 |
| 455063 | 1094851_1 | AW854160 AW854156 AW854149 AW854147 AW854158 |
| 406827 | 0_0 | AA971409 |
| 400157 | 2764_1 | BC022339 BC009610 BC010537 X79805 NM_006713 U12979 BM467814 BM450743 AU132951 |
| | | AU137129 BG493425 AV758819 BG708412 |
| | | BG705885 BG702217 AV716638 BG777009 BI545689 BI552153 BM476712 BG770858 BG527656 BG528277 BG391388 |
| | | AV716861 BI602926 |
| | | BG290073 BI667399 BM451469 BI667173 BI602139 BG532171 BI669216 BI544727 BG721852 AV716503 AV701327 |
| | | BM090738 BI492000 |
| | | AI308856 BI544904 AL599813 AV715829 AV716505 AV714587 AV717902 BF668072 AV716385 BI461927 BM090954 |
| | | AV717826 BG503676 |
| | | AV647719 BG501392 BG428433 BE895629 BM313117 AW021050 BG435032 BM152910 AA313503 AA872377 BG574714 |
| | | AV712054 AV732696 |
| | | AA252476 AV712759 AL599643 BE790872 BG654930 W73337 AW675377 AV760376 AV725139 AV716379 AA887165 |
| | | BE830003 AW023796 |
| | | AL599291 AI902948 BG944042 F00781 AA352483 BG217897 N33888 AW581924 BG654730 D31410 AA353088 D31288 |
| | | AA295029 H95170 |
| | | BE935104 AU139980 BG772963 BG776470 BG532512 BG105449 BI545421 AV715456 AW386083 BG699714 AL535832 |
| | | AL514940 BG190861 |
| | | BG210593 AW999254 H95138 AA353863 BE764809 N50375 BE091363 BG701255 BI860846 BI832485 BG168150 |
| | | BG028647 BE546301 |
| | | BG900321 BI909737 BG702363 BG614141 BG611137 BG700121 BF031492 N85802 AV715940 N51590 BG993478 |
| | | BE172016 AW893622 |
| 449261 | 3029652_1 | AI923721 AI860294 AI637592 |
| 434636 | 15423_1 | AJ420454 AF147430 AA910497 BF432963 AI701451 AI743089 AA429326 AI887812 AA315932 AI005464 AL043321 |
| | | AI300993 AA425105 |
| | | BE467230 BE669770 AA885637 BE503044 AW014324 AI809584 AW167510 AA921331 AA903224 W01644 AI762128 |
| | | AA031404 BE550653 |
| | | AI694045 BE043088 BE670430 AI630969 AI457315 BE644737 BE327316 AW295247 N92784 AI630807 BE328180 |
| | | AI269949 AW245292 |
| | | AA083765 AA256898 AI375535 AA430673 AI168735 AI589717 AA015942 AI693885 AW341205 AA931651 BF856764 |
| | | BE468094 BF433393 |
| | | BF445511 AA928976 AI817684 BF111008 AA428316 AA455858 N25716 AA568727 AI581817 AA427482 H40678 |
| | | AA041483 N71630 H51826 |
| | | BF969052 AA094470 AI560352 T98937 W52816 AA083764 |
| 413186 | 10095_1 | AI800480 AI675873 AI492036 AA285369 AA043133 AI565092 AI094934 AI127591 AA774663 AW262141 |
| | | AW184007 AI423287 AI282115 |
| | | AI309111 AA206116 AI984711 AI218432 AA565473 AW439256 AI915825 AI690350 AI867581 AA888739 AW135142 |
| | | BG202056 Z39676 T16640 |
| | | AA765047 AW020940 AI961664 AI636487 AA492375 BF511091 BF511214 AI129459 AW117212 AI968323 AI656300 |
| | | BE503348 BE503313 |
| | | AI361209 AI925328 AA968416 AW611654 AW614387 BG108803 AA029159 BF176324 R16207 AL567126 AA224460 |
| | | BM145135 BE855388 |
| | | BI491899 BI493821 AL581038 R49543 AA422156 H92081 AA325525 R66331 BF748472 AI990318 AA279568 R67472 |
| | | R32365 AI361184 R66332 |
| | | AA029056 BE738339 BM145028 D29452 BE619671 R80926 AA130505 AW271168 |
| 417890 | 6087_3 | NM_003777 AJ320497 BF980177 AA489276 AI042319 BF881839 BG999412 BI003305 |
| | | W31815 AJ132087 BF369731 BG186912 AI740921 |
| | | AI453059 AI027448 AI027859 AI041705 AI087934 BF056332 BF109708 AA937198 W20023 R23111 R79048 N90533 |
| 453823 | 1489490_1 | AL137967 BE064160 BE064186 |
| 436763 | 1050498_1 | BG928040 AI095270 AI990086 AI991608 AA730741 AA868238 BE815056 BE550792 AI168278 AI522194 AA973538 |
| | | AI628424 BE814976 AI819707 |
| 446002 | 2852062_1 | AI346468 AI347958 AI269775 AI269780 |
| 409835 | 915194_1 | AW501362 AW501275 |
| 411111 | 1070063_1 | AW818161 R09719 AW818127 |
| 408594 | 685806_1 | BE184942 BE184946 AW238414 BE144666 |
| 433361 | 25962_1 | AF026526 BG210339 AW611839 AI333926 AI719252 AI478251 AW469373 AI678201 BF056830 BE552206 AA706678 |
| | | AW594061 AI744564 |
| | | AI025408 AI744629 AI033875 AI051333 BF438997 AI991433 |
| 442079 | 648638_1 | AL046653 AW183787 AA977613 AA975487 AI051504 |
| 443274 | 33097_1 | AK023535 BI850449 BI494019 BG113829 BI494020 BF378857 AW262721 AI339175 AW029243 AI417477 AI679401 |
| | | AI083840 BF475695 |
| | | F26528 AI679908 AA659612 AI453737 AI523378 AW630773 AA251578 AA658039 R72090 AI832404 AV693821 |
| | | AV698231 AV685465 |
| | | BM423386 R80276 W39196 BI258049 AA508602 T85159 AA156499 AA664240 BF212163 AU156501 |
| 413840 | 38807_1 | BC020284 AW953495 AW402677 BF890739 Z44378 AI660081 AI769242 T84954 C05886 AI224851 AI568340 AI024026 |
| | | AW015010 AI032051 |
| | | AA505095 AA830304 H98001 H63431 BG249610 AI284792 AA205732 AI598130 AW516813 AI123905 |
| | | AA132557 AI140199 |
| | | H53399 AA928496 |
| | | AI200299 AI400044 BE940512 BE940609 H28028 BF913868 AI301558 D79095 AI762695 AA311547 AI673408 |
| | | AI673002 N67358 AA885913 |
| | | AI288094 N67347 Z40311 T94918 AV722693 AW170339 AW975566 AA452774 AA504759 AA370643 T79706 AA721603 |
| | | BF858887 BG982917 |
| | | AI343373 AW236167 BE568091 BF747959 BF241262 BE940663 AI435278 R05794 AW027091 AI860259 AI268890 |
| | | T94871 AW182884 N67183 |
| | | AW149083 H42473 AI678254 R26706 AI419684 R24905 AW388568 AW363261 N31299 H63489 AW195475 AA452592 |
| | | BE844216 T91205 |
| | | H64955 BF858811 AW962779 AW388295 BF857615 BF856552 BF856548 BF857611 AW866752 |
| 430715 | 32242_1 | AI148194 |
| 450234 | 707416_1 | AI797978 AI688524 AW270069 |
| 454514 | 1058176_1 | AW802838 BE170861 |
| 449540 | 79180_1 | AA001803 H63836 AA001713 H63784 |
| 444156 | 7612_13 | BM456075 AA287437 AW503282 AW500236 AW505532 BM476391 BM151675 AW500059 AW503519 |
| | | AW505387 AV760244 BM463484 |
| | | AW499930 AW499907 AW504121 AW504494 AA286674 AI276686 AA286673 AW407901 AW503798 BG177295 |
| | | BG389213 BM145086 |
| 454552 | 1061234_1 | AW806924 AW866478 AW866473 AW866390 AW866309 BF373982 AW866298 AW866539 AW866521 AW866547 |
| | | AW866454 AW866537 |
| | | AW866517 BF373957 BF373949 AW866403 AW866369 |
| 417414 | 10043_1 | AB049113 U31930 NM_001948 U90223 AK000629 BI091680 BG697899 BG700386 BE644721 BE673841 BE670064 |
| | | BE218639 AW160384 |
| | | BI489866 AW341118 AW629827 AI697600 BE222283 BF938984 BE550008 BF058963 BE549576 BE504213 AI951891 |
| | | BG654389 AW162006 |
| | | BE551158 BG655751 BI438412 AI634515 AW967946 AI373097 AI937879 BF477872 AI693247 AA284034 |
| | | AW968574 AW968748 AL519489 |
| | | AA491130 M89913 BG403290 M504296 AA263067 AA366402 BF899218 AW007239 AU119115 D55898 BI333317 |
| | | AI879028 AV651114 |
| | | BE502892 AW341323 BF478012 BG705392 BG505144 AV747151 AA416647 W65362 BF170355 AI361859 AI061446 |
| | | AA744361 AI800124 |
| | | AI888707 AI810305 AW950219 AI266370 AI202472 AA731309 AI352257 BE327789 D19871 AW059539 BM263848 |
| | | AI828180 AA994216 |
| | | AA721362 R76541 AA806886 AA861025 AA687747 AI304562 BI091131 BF206146 BI255334 BE254729 BG610639 |
| | | AA278989 BF698002 |
| | | AA446533 BF970255 BE928140 AI348028 AI521455 AW474561 AW591403 AI654314 AV701730 AI433388 AW166689 |
| | | AW976899 BE568767 |
| | | AA887905 AA766415 BG168154 BF317403 BI855761 BI758597 BF216176 |
| 453959 | 9900_1 | NA |
| 439195 | 21979_1 | AF086037 H89360 H89546 |
| 400223 | 2368_1 | NM_005648 BC013809 L34587 BF103775 BG702618 BG716553 BI667090 BG505863 |
| | | BF983483 BG718195 BI857891 BG501016 BM043599 |
| | | AL521812 BG705730 BI495545 BI495546 BF112248 BM023182 BM023123 AI075173 AW051799 BF058224 |
| | | BI324885 BF436008 AA398446 |
| | | BG822375 BM019558 BM023382 BG164174 N56909 BI467064 BM023464 AI207475 BM311415 BG758430 BG758807 |
| | | AI934826 N90351 |
| | | BG422026 BE910312 AI027778 AI081950 AI360890 BM009115 AI191829 BG759697 AI138728 AA399403 AI355589 |
| | | AI336427 AA868702 |
| | | AA393660 AA025127 BG027630 AA962774 AA631224 BG940967 BE791087 AA573315 W81685 AA393525 BG944103 |
| | | AI339125 AI149864 |
| | | AA977655 N90314 BE612839 BG491847 AI129091 AA461234 AA781198 AA759256 AA888954 AA975844 AI184099 |
| | | AI018025 AA398363 |
| | | AI003331 AI193380 AA626020 AI244476 AI601114 AW135664 AI206607 AW263599 AA813219 AI684453 AA878626 |
| | | AA772222 AI085496 |
| | | AI630226 BG940966 AI022010 AA770649 AA887624 AA491739 AA974295 BG530040 AA037091 AA019912 BI160457 |
| | | H64512 BG503896 |
| 454067 | 98108_1 | AI809849 AW241782 AA041455 |
| 442092 | 22756_2 | BM449821 BM069895 BM023170 AW025563 BG152606 BM023452 AI862106 BG959957 |
| 430504 | 5477_6 | BE219720 BF475241 AI571723 BE219848 BI789268 AI224899 AA724864 AW771467 AA480255 AW845616 |
| | | AI440295 H52800 BE218790 |
| | | AI681575 AW300064 AW262133 H21568 AI363015 AI884914 H86948 |
| 406731 | 0_0 | AI559131 |
| 457447 | 44331_1 | X78261 AI214094 AA483530 AW972127 |
| 455679 | 151610_1 | BG007296 BF330853 BF747375 BE066356 BE066292 BF330900 BF747142 BE066419 BF742510 BE066529 |
| | | BE066298 BF742516 BF746603 |
| | | BE066274 BF334312 |
| 430534 | 1233361_1 | AW968508 AA480908 AA480971 |
| 409490 | 2925095_1 | AA084745 AA084736 AA075064 |
| 425376 | 34867_1 | AB075838 AK056181 AW958603 BE390381 AA356365 BF196303 H10404 Z39522 AV725320 AV727868 BM454236 |
| | | AA365277 R38602 H10612 |
| | | BF212958 AA481673 R13757 BG026236 AI498819 AA894612 AA610077 AW352149 AA481433 AW079577 AI885666 |
| | | AA532463 AI624958 |
| | | AI624974 H93131 BE566327 AI955749 AI478116 BE546266 |
| 437440 | 2497201_1 | AA846804 AA757581 AI050950 AI092024 AA838807 |
| 453327 | 23886_2 | AL562496 BF696621 AV704702 BG898086 AW968374 AA481612 AA431838 AA476509 BM271915 BI438471 AW021944 |
| | | AA465428 BI492903 |
| | | AW500180 AI337886 AW628927 AI337904 AI865657 AI095827 AI277333 AI192671 M910864 AI056123 AI359717 |
| | | AI801670 AA554891 |
| | | BF002895 AI146699 AI277967 AI421245 AI554684 AI076969 AA127232 AI090547 AW295617 AI339523 AI144431 |
| | | AA857201 AA476409 |
| | | AA465393 BF445672 AI247616 AA905002 AA723681 AA465357 AA465372 AI027015 BF939274 BE769036 AA983718 |
| | | AI796838 BF373700 |
| | | AW864097 AA506221 BE326573 AW368726 AA035002 AA149487 BE887617 AI692779 AI183608 AA858044 BI467397 |
| | | AA046036 W67790 |
| | | W32699 AA431436 AA035510 AA836772 AA481546 BM264551 AA465448 AA339752 BF698980 AA621703 BE825936 |
| | | W67848 BE766805 |
| | | N66434 N59817 N26170 AA668310 BE768929 |
| 428384 | 775_1 | M54968 AA861543 AA356548 AI469981 NM_004985 BC013572 AW242010 AI740449 BF588730 AA425263 |
| | | W21091 AI554920 T28976 |
| | | AW966530 BC010502 BM146110 AW504574 BG142117 AA232870 AI539465 AU123937 BI260541 BG261289 N24354 |
| | | N32462 H08297 F05452 |
| | | AA281203 N80109 AA330460 D51551 BE178107 BE177839 BE178094 BE178141 BE931948 BE178093 BE178215 |
| | | AL138354 AI091259 |
| | | AW020801 AA287991 BE178352 AA281183 BE178186 R70669 BI833742 BE178449 BE178043 W47526 BE178018 |
| | | BE834453 W93668 |
| | | BE177936 AL576273 AU151852 AI004579 AW453052 AA287927 BM144801 AU155143 AA281094 AA737010 AA872481 |
| | | AA676681 AA843916 |
| | | AI697660 AA564243 BF109372 AI015987 BF432094 AA937541 AI334416 AI281159 AA582189 AA553559 AW160515 |
| | | AA255527 AA670007 |
| | | AA808271 AA281015 AA649252 BE464958 AW167917 BF436494 AA525301 AI889481 AI364302 AW173466 AI676214 |
| | | AA243624 AA648116 |
| | | AA854442 AA721296 F01713 BE178360 BE178299 BE178460 BE178385 BE178424 AI301629 BE049265 AU151277 |
| | | AU155121 AU155337 |
| | | AA889246 AU153181 AA342730 AW601642 H08199 R41588 R99351 H84241 W47527 R26447 R49696 H02312 R79833 |
| | | AV716970 AA262387 |
| | | BE178441 BE178356 BE178100 BI491761 BE178467 BE931952 BF758698 BE177893 BE178336 AA256711 W80934 |
| | | BE178447 AV689618 |
| | | BE178438 BE177844 AV659669 BG619554 AU134516 R35396 BE178029 R28455 AW368425 BE178017 BE177999 |
| | | R34165 R79640 BE178422 |
| | | BF761849 BE178196 BE178443 |
| 426295 | 510_1 | BE880923 BG390191 AW470082 AW014585 AI423255 BI714731 BG054894 AW780248 N31683 AW664132 |
| | | AW467353 AI983152 AA617918 |
| | | BF447795 AI088357 AA807328 AA576970 AI741153 AI755003 AI474016 AI422030 AI348114 AW997085 BM271753 |
| | | AI363147 BM311311 |
| | | AI146640 AI246771 AW512619 AI359020 BG054897 AI292234 AI215830 AI283836 C06205 AW503423 AW272680 |
| | | N33205 AW873021 |
| | | AA070724 AI753886 AW192487 AI087151 AA658909 AI346368 AI335677 AA825442 AW440066 AW131357 AW513210 |
| | | AI082314 AI085455 |
| | | BE551404 AA780704 AW008596 AI796964 AA917471 AI400531 AA668626 N72207 AI306482 AW440562 AI084687 |
| | | AA347280 AA063536 |
| | | BF477389 AI241662 AA931543 AA484310 AA812486 AI032216 AA665779 AI916336 AI350590 BF198106 AI433377 |
| | | AI300638 AI672626 |
| | | AI282741 AI351487 AW105544 AA973627 AW517914 AA715424 AA508454 BF334080 AI274618 AW367201 AW572619 |
| | | AW469088 AA382095 |
| | | AI368364 AI146934 AI357180 AI361181 BI911347 BI871044 AA136325 BF084010 BF084007 AA335788 AI920878 |
| | | AA809614 BE932941 |
| | | AI678261 C75308 AI148479 BE178174 W88513 BM013627 |
| 436024 | 138548_1 | AI800041 AA703553 AA984529 |
| 419881 | 3973_1 | AL573520 BF058611 AA329340 AL578426 AA748587 AA251524 BG056795 AA989242 AA716558 BI860141 |
| | | AW967619 AI949529 AI889732 |
| | | AI871690 AA830473 AA769808 AA570708 AA844702 AA838659 AI653883 AW768563 AA442846 AW273448 AA437017 |
| | | AI493986 AW628781 |
| | | AL562775 AI870399 AI432295 AW439601 |
| 414022 | 32320_46 | W44916 F07627 |
| 415524 | 1875412_1 | R13958 Z45262 F11541 |
| 459428 | 259201_1 | AW502680 AA258989 |
| 412634 | 131633_1 | U55984 AA195883 AA447957 AA393452 AA384799 AW958952 W68044 AA197345 AI241933 |
| | | R59505 W67965 AA826474 AI336006 AA931733 |
| | | AW025414 AI769180 AI355738 AI860952 AA907417 AW408679 AA453139 AA938346 AI279697 AA121899 AA400768 |
| | | AI559827 AA448889 |
| | | N62400 AW236656 AA904207 W79380 R59504 |
| 416721 | 1987834_1 | H80001 H87290 |
| 454900 | 1077113_1 | AW838132 AW838129 AW838458 AW838513 AW838430 AW838373 |
| 442462 | 1984399_1 | AF031405 H73415 |
| 434665 | 115504_1 | AA642125 BG311079 BG311075 AA654516 BG311138 BG311173 |
| 400239 | 16458_1 | BC003160 NM_001610 X12548 BG386685 BI760866 BI559619 BG323829 AU135543 BI834101 AU142120 |
| | | AU124511 AU124889 AL558171 |
| | | AU117286 BI824000 BG386610 BI753285 BI223475 AU134828 BM126369 BE206493 BF751498 AL544274 AY007137 |
| | | BI828921 BE870130 |
| | | BF771242 BI835451 BI765655 BI820955 BI223344 BG015924 BI759894 AL527413 BF310588 N31870 N23974 BE514914 |
| | | T48863 AW860257 |
| | | BF334625 AA883860 AU144168 AA442562 AU159491 AU148353 AA564123 AU148667 AI377256 AW664004 AI871712 |
| | | AI141486 AI332351 |
| | | AI339094 BE206109 AW519033 AI817729 AI332490 AI149455 AI857411 AI763154 AI751608 AI377222 AI081956 |
| | | AW664229 AI275872 |
| | | AW168546 AA975270 AI367408 AI687729 AI269164 AW105344 BM193081 BE550930 AI082116 AA854691 AI056249 |
| | | AI221062 AI290113 |
| | | D51818 AA732409 BG055125 N85878 AU156121 BF093671 AA053070 T28548 AL570460 BI006862 BI007207 AA373620 |
| | | W95069 AA629136 |
| | | AA708164 AI014886 AW168697 D51623 AA577168 AI094208 AA028946 AA975078 R16815 BG151132 AI276297 D51676 |
| 455148 | 1099439_1 | AW858423 AW858403 AW858594 AW858406 AW858587 AW858549 |
| 434775 | 118125_1 | AA648983 AA741088 AW029205 AI925642 AA715292 |

Pkey: Unique Eos probeset identifier number
CAT number: Gene cluster number
Accession: Genbank accession numbers

TABLE 11C

| Pkey | Ref | Strand | Nt_position |
|---|---|---|---|
| 402994 | 2996643 | Minus | 4727-4969 |
| 402264 | 3289984 | Plus | 184006-185825 |
| 402424 | 9796344 | Minus | 64925-65073 |
| 404649 | 9796926 | Minus | 100027-100399 |
| 405533 | 9755498 | Minus | 75690-75853 |
| 402230 | 9966312 | Minus | 29782-29932 |
| 400751 | 7331445 | Minus | 35395-35533 |
| 400694 | 8118802 | Plus | 94288-94442 |
| 401467 | 6682292 | Minus | 66845-67037 |
| 403789 | 8084957 | Minus | 64056-64148, 65262-65425 |
| 406117 | 9142932 | Plus | 54304-54584 |
| 406420 | 9256411 | Plus | 157797-158054 |
| 404426 | 7407959 | Plus | 77842-77954 |
| 401274 | 8954206 | Minus | 111258-111378 |
| 400929 | 7651921 | Minus | 122033-122241, 123483-124028 |
| 403790 | 8084957 | Minus | 87826-87947, 89835-90002 |
| 405268 | 4156151 | Minus | 24404-24521 |
| 402855 | 9662953 | Minus | 59763-59909 |
| 400517 | 9796686 | Minus | 49996-50346 |
| 402976 | 9795206 | Minus | 25819-25969 |
| 403743 | 7652003 | Minus | 136463-136646 |
| 403828 | 9838214 | Plus | 31755-32148 |
| 405689 | 4508117 | Minus | 92558-92698, 94282-94382, |
| | | | 97977-98180, 9920 |
| 403742 | 7212067 | Plus | 84805-85525, 91096-91227, |
| | | | 93970-94057, 9523 |
| 403319 | 8318526 | Plus | 124240-124459, 125182-125409 |
| 404602 | 8705107 | Minus | 138628-138893 |
| 403654 | 8736093 | Minus | 28634-28758 |
| 402396 | 1905896 | Plus | 4426-4648 |
| 405372 | 2078459 | Minus | 10148-10272, 11205-11349, |
| | | | 11436-11560, 1178 |
| 403694 | 7107765 | Plus | 142925-143080, |
| | | | 165505-166186, 167486-16763 |
| 404584 | 9857511 | Plus | 138651-139153 |
| 404240 | 5002624 | Minus | 116132-116407, 116653-116922 |
| 404409 | 7342122 | Plus | 22113-22290 |
| 405601 | 5815493 | Minus | 147835-147935, 149220-149299 |
| 404919 | 7341872 | Plus | 1482-1609 |
| 403738 | 7212067 | Plus | 38434-38562, 56876-57007, |
| | | | 59789-59876, 6071 |
| 401847 | 7139731 | Plus | 85447-85593 |
| 401928 | 3873182 | Plus | 54932-55070 |
| 406274 | 7543787 | Plus | 932-1123 |
| 401558 | 7139678 | Plus | 103510-104090 |
| 403746 | 7652036 | Plus | 93612-93887 |
| 402945 | 9368458 | Minus | 100591-100710 |
| 400487 | 8919452 | Plus | 19369-20782 |
| 403330 | 8516153 | Plus | 116558-116698 |
| 400975 | 7139779 | Minus | 108473-108847 |
| 400552 | 9801071 | Plus | 93101-93276 |
| 403147 | 9799812 | Plus | 171145-171419 |
| 406605 | 8272666 | Minus | 23275-23493, 23723-23903 |
| 402907 | 6706902 | Minus | 8561-8692 |
| 401431 | 8217913 | Plus | 81467-81661 |
| 401047 | 6705887 | Minus | 4804-5035, 5133-5314 |
| 404813 | 3355600 | Plus | 2672-2989 |
| 406137 | 9166422 | Minus | 30487-31058 |
| 400763 | 8131616 | Minus | 35537-35784 |
| 403608 | 8308266 | Minus | 121321-121476 |
| 400840 | 9188586 | Plus | 113882-114121 |
| 403827 | 9838214 | Minus | 14941-15869 |
| 403809 | 8568861 | Plus | 33910-34129, 34583-34862 |
| 403142 | 9444521 | Plus | 89286-90131 |
| 402813 | 6714723 | Minus | 45613-45781, 47581-47731, |
| | | | 49698-49805, 5095 |
| 403538 | 8077008 | Minus | 119579-119926, 120614-120784 |
| 402969 | 9581784 | Minus | 19036-19175 |
| 406182 | 5923650 | Minus | 28256-28935 |
| 406364 | 9256114 | Minus | 50715-50833 |
| 405232 | 7249042 | Plus | 125904-126063 |
| 403417 | 6862691 | Plus | 199375-199572 |
| 405257 | 7329310 | Plus | 73121-73273 |
| 403585 | 8101208 | Minus | 131266-131769 |
| 403832 | 2121307 | Plus | 76091-77344 |
| 402964 | 9581599 | Minus | 46624-46784 |
| 401060 | 8117674 | Minus | 110262-110791 |
| 406350 | 9256015 | Minus | 186117-186362, 190904-191095 |
| 403056 | 8954178 | Plus | 95479-95570, 96434-96703 |
| 406341 | 9255965 | Minus | 117712-117896 |
| 404694 | 9799957 | Minus | 128092-128227 |
| 402668 | 7283250 | Plus | 67493-68044 |
| 405296 | 3779036 | Plus | 8072-8273 |
| 402729 | 9211639 | Plus | 78980-79093, 79333-79467, |
| | | | 80173-80290 |
| 405122 | 8137462 | Minus | 95449-95646 |
| 402737 | 9212184 | Minus | 13358-13552 |
| 404231 | 8218035 | Minus | 61077-61322 |
| 405287 | 3928029 | Plus | 89802-89999 |
| 405695 | 4309958 | Plus | 51860-52162 |
| 401112 | 9966198 | Minus | 60628-61041 |
| 403671 | 7272159 | Plus | 104461-104701 |
| 400886 | 9958187 | Plus | 70649-71114 |
| 402474 | 7547175 | Minus | 53526-53628, 55755-55920, |
| | | | 57530-57757 |
| 405064 | 7658416 | Plus | 81207-81416 |
| 402007 | 7381786 | Plus | 143964-144081 |
| 400967 | 7770682 | Minus | 32697-32999 |
| 404497 | 8151650 | Minus | 593-1416 |
| 404692 | 9799952 | Plus | 49258-49403, 54226-54341, |
| | | | 63837-63988 |
| 405166 | 9966302 | Plus | 40526-40891 |
| 405270 | 4156145 | Minus | 3952-4123, 6886-7010, 8541-8728 |
| 402109 | 8131678 | Minus | 171722-171859, 173197-173303 |
| 403363 | 8571785 | Plus | 67156-67285, 67620-68007 |
| 406023 | 8272661 | Plus | 205623-205936 |
| 403185 | 9838287 | Minus | 108676-108803 |
| 403611 | 8347965 | Minus | 130635-130774 |
| 403707 | 7108128 | Minus | 132794-133294 |
| 404124 | 9796734 | Plus | 13539-14192 |
| 403292 | 7230870 | Plus | 99123-99367 |
| 403027 | 7670575 | Plus | 60696-60932, 61362-61521 |
| 406535 | 7711477 | Plus | 83135-83362 |
| 405922 | 6758795 | Plus | 126307-126677 |
| 406238 | 7417725 | Plus | 31155-31657 |

Pkey: Unique number corresponding to an Eos probeset
Ref: Sequence source. The 7 digit numbers in this column are Genbank Identifier (GI) numbers. “Dunham, et al.” refers to the publication entitled “The DNA sequence of human chromosome 22” Dunham, et al. (1999) Nature 402: 489-495.
Strand: Indicates DNA strand from which exons were predicted.
Nt_position: Indicates nucleotide positions of predicted exons.

Table 12A lists about 254 genes down-regulated in Hepatitis C positive liver tissues compared to Hepatitis C negative liver tissues. These genes have the potential to be diagnostics and/or prognostic markers for Hepatitis C infected liver tissues. They may also provide clinical information on Hepatitis C infection and pathology. They may also be potential targets for therapeutic drugs and/or treatments. These were selected from 59680 probesets on the Affymetrix/Eos Hu03 GeneChip array such that the t-test p-value between the Hepatitis C positive liver tissues group and the Hepatitis C negative liver tissues was less than 0.01, and that the differences between the average “standardized values” of the several different Hepatitis C negative liver tissues was equal to or above 1.0 more than the average “standardized values” of the several different Hepatitis C positive liver tissues. The “standardized values” of the Hepatitis C liver tissues were derived by subtracting the median of the Hepatitis C negative liver tissue values from each probeset value, then divided by the interquantile range (IQR) of the same Hepatitis C negative liver tissue values.

TABLE 12A

| Pkey | ExAccn | UniGene | Unigene Title | R1 | R2 |
|---|---|---|---|---|---|
| 441475 | AI929602 | Hs. 177 | phosphatidylinositol glycan, class H | 6.80E−05 | 9.91 |
| 453863 | X02544 | Hs. 572 | orosomucoid 1 | 1.46E−03 | 5.21 |
| 449761 | AB009698 | Hs. 23965 | solute carrier family 22 (organic anion | 2.10E−03 | 4.68 |
| 445610 | AI831648 | Hs. 143993 | ESTs | 1.53E−03 | 4.63 |
| 404473 | | | ENSP00000247423: D-siglec precursor. | 3.16E−03 | 4.40 |
| 440721 | AW183540 | Hs. 85273 | ESTs | 9.60E−05 | 4.14 |
| 442820 | AW293459 | Hs. 6314 | ESTs | 1.50E−05 | 4.04 |
| 446221 | AI744445 | Hs. 167073 | Homo sapiens cDNA FLJ13047 fis, clone NT | 7.92E−05 | 3.41 |
| 449184 | AW296295 | Hs. 196491 | ESTs | 5.21E−10 | 3.23 |
| 454947 | AW846590 | | gb: QV0-CT0180-011099-025-d07 CT0180 Homo | 1.84E−06 | 3.21 |
| 434180 | AA921757 | Hs. 116180 | ESTs | 7.08E−07 | 3.01 |
| 423744 | D26158 | Hs. 1701 | ELAV (embryonic lethal, abnormal vision, | 2.94E−03 | 3.00 |
| 410482 | AW772187 | Hs. 191859 | ESTs | 7.60E−06 | 2.93 |
| 422472 | R59096 | Hs. 279939 | mitochondrial carrier homolog 1 | 9.77E−05 | 2.89 |
| 454136 | AA046477 | Hs. 62813 | insulinoma-associated protein IA-6 | 4.63E−04 | 2.89 |
| 458818 | AI523857 | Hs. 232257 | ESTs | 4.91E−05 | 2.87 |
| 457960 | AA771881 | Hs. 298149 | ESTs | 3.65E−03 | 2.84 |
| 446433 | AW293404 | Hs. 211986 | ESTs | 1.75E−04 | 2.73 |
| 409212 | AI082423 | Hs. 141892 | ESTs | 1.60E−06 | 2.72 |
| 435590 | AF216389 | Hs. 148932 | sema domain, transmembrane domain (TM), | 8.04E−05 | 2.72 |
| 402845 | | | ENSP00000246267: KIAA0444 PROTEIN (FRAGME | 9.30E−06 | 2.67 |
| 400692 | | | Target Exon | 4.42E−10 | 2.67 |
| 411449 | AW847318 | Hs. 290131 | KIAA1819 protein | 1.36E−04 | 2.66 |
| 455765 | BE082222 | | gb: QV2-BT0636-070500-196-b08 BT0636 Homo | 2.68E−03 | 2.64 |
| 458674 | AI304386 | Hs. 150836 | ESTs | 5.42E−07 | 2.59 |
| 455919 | BE157345 | | gb: RC3-HT0371-110300-017-b01 HT0371 Homo | 6.41E−04 | 2.58 |
| 412998 | BE046254 | | gb: hn38g09.x2 NC_CGAP_RDF2 Homo sapiens | 6.95E−05 | 2.56 |
| 417574 | R00348 | | gb: ye69e06.r1 Soares fetal liver spleen | 1.30E−03 | 2.53 |
| 437957 | T97069 | Hs. 282787 | gb: ye50f03.r1 Soares fetal liver spleen | 7.21E−08 | 2.51 |
| 452777 | AW449161 | Hs. 217300 | ESTs | 2.81E−07 | 2.50 |
| 428547 | AF120491 | Hs. 184889 | potassium voltage-gated channel, Shal-re | 3.13E−04 | 2.49 |
| 435715 | T78013 | Hs. 167279 | FYVE-finger-containing Rab5 effector pro | 2.14E−04 | 2.48 |
| 456636 | AW205448 | | complement component 3 | 1.09E−05 | 2.46 |
| 432342 | AL036128 | Hs. 274404 | plasminogen activator, tissue | 3.57E−05 | 2.46 |
| 427269 | AA400147 | Hs. 125234 | chromosome 21 open reading frame 54 | 2.37E−07 | 2.42 |
| 409840 | AW502122 | | gb: UI-HF-BR0p-ajr-c-08-0-UI.r1NIH_MGC_5 | 2.53E−06 | 2.42 |
| 414562 | AW955734 | Hs. 112195 | ESTs, Weakly similar to 2108402A carniti | 1.68E−05 | 2.41 |
| 420796 | L34355 | Hs. 99931 | sarcoglycan, alpha (50 kD dystrophin-asso | 3.13E−03 | 2.38 |
| 432745 | AI821926 | | gb: nt78f05.x5 NCI_CGAP_Pr3 Homo sapiens | 1.10E−04 | 2.37 |
| 428006 | AA418743 | Hs. 98306 | KIAA1862 protein | 8.66E−04 | 2.35 |
| 407563 | R06177 | Hs. 19613 | ESTs | 1.89E−08 | 2.34 |
| 400435 | AF282973 | | protocadherin beta 8 | 1.15E−05 | 2.32 |
| 430878 | BE500985 | Hs. 248107 | EDG-3 (endothelial differentiation, sph | 2.20E−05 | 2.30 |
| 413946 | BE185066 | Hs. 2055 | gb: MR1-HT0709-100500-002-c09 HT0709 Homo | 1.67E−03 | 2.30 |
| 437015 | AI970163 | Hs. 237198 | ESTs | 3.21E−03 | 2.27 |
| 458988 | AW410431 | Hs. 283670 | CGI-119 protein | 9.83E−06 | 2.26 |
| 454810 | AW832917 | | gb: QV2-TT0003-161199-013-h06 TT0003 Homo | 3.07E−03 | 2.24 |
| 458299 | AI024834 | Hs. 131729 | ESTs | 2.45E−06 | 2.22 |
| 438397 | AA806478 | Hs. 123206 | ESTs | 1.20E−04 | 2.22 |
| 404224 | | | Target Exon | 2.72E−10 | 2.22 |
| 405800 | | | Target Exon | 1.33E−06 | 2.22 |
| 411772 | BE170301 | | gb: QV4-HT0536-040500-193-f05 HT0536 Homo | 5.16E−04 | 2.21 |
| 401149 | | | Target Exon | 9.04E−07 | 2.20 |
| 405334 | | | Target Exon | 3.80E−04 | 2.20 |
| 441611 | AW590829 | Hs. 133463 | ESTs | 1.73E−08 | 2.19 |
| 407457 | AJ242724 | | gb: Homo sapiens mRNA for partial putativ | 4.71E−05 | 2.18 |
| 414024 | AA134712 | Hs. 22410 | gb: zm79g08.r1 Stratagene neuroepithelium | 3.72E−07 | 2.18 |
| 450703 | AA011202 | | nuclear factor I/C (CCAAT-binding transc | 1.02E−06 | 2.17 |
| 436871 | X15667 | | gb: Human mRNA for glutathione peroxidase | 3.18E−07 | 2.16 |
| 417752 | C15737 | Hs. 269386 | ESTs | 2.59E−05 | 2.16 |
| 404958 | | | Target Exon | 8.05E−05 | 2.16 |
| 459235 | BE246010 | | Homo sapiens mRNA for FLJ00038 protein. | 3.75E−05 | 2.14 |
| 416626 | H69171 | Hs. 205978 | ESTs, Moderately similar to ZN91_HUMAN Z | 3.33E−03 | 2.14 |
| 419695 | N99713 | Hs. 109514 | ESTs | 6.58E−04 | 2.13 |
| 412496 | R87592 | Hs. 12509 | gb: ym91b06.r1 Soares adult brain N2b4HB5 | 9.12E−05 | 2.12 |
| 426505 | BE185582 | | gb: IL5-HT0731-010500-078-g07 HT0731 Homo | 7.94E−04 | 2.12 |
| 453840 | AW846927 | Hs. 46808 | gb: RC3-CT0197-150999-011-A01 CT0197 Homo | 1.36E−04 | 2.12 |
| 423053 | BE312679 | | gb: 601148138F1 NIH_MGC_19 Homo sapiens c | 1.00E−05 | 2.12 |
| 436690 | AA373970 | Hs. 183096 | ESTs, Highly similar to S15536 homeotic | 9.53E−05 | 2.11 |
| 405058 | | | Target Exon | 5.01E−07 | 2.11 |
| 426402 | BE387327 | Hs. 80475 | polymerase (RNA) II (DNA directed) polyp | 3.46E−06 | 2.10 |
| 449243 | AW295031 | Hs. 198671 | ESTs | 1.86E−05 | 2.10 |
| 444163 | AI126098 | | FGENESH predicted RNaseH domain-containi | 6.37E−04 | 2.10 |
| 440730 | BE561722 | | gb: 601345466F1 NIH_MGC_8 Homo sapiens cD | 5.62E−05 | 2.09 |
| 437161 | AA054477 | Hs. 25391 | ESTs | 9.90E−05 | 2.08 |
| 447721 | BE619620 | | lysyl oxidase | 1.33E−06 | 2.08 |
| 455973 | BE175424 | | gb: RC4-HT0578-170300-012-d01 HT0578 Homo | 1.01E−03 | 2.06 |
| 407598 | AW083834 | Hs. 246397 | ESTs | 3.87E−03 | 2.06 |
| 457736 | AK000390 | Hs. 150549 | hypothetical protein FLJ20124 | 2.25E−03 | 2.05 |
| 433303 | AW444881 | Hs. 77829 | ESTs | 1.28E−05 | 2.04 |
| 456040 | AW390834 | Hs. 75874 | pregnancy-associated plasma protein A | 2.69E−04 | 2.04 |
| 454707 | AW814989 | | gb: MR1-ST0206-170400-024-g05 ST0206 Homo | 2.84E−04 | 2.04 |
| 410739 | AL157438 | Hs. 324473 | Homo sapiens mRNA; cDNA DKFZp434A115 (fr | 4.80E−03 | 2.04 |
| 430070 | AF197927 | Hs. 231967 | ALL1 fused gene from 5q31 | 1.38E−06 | 2.03 |
| 452621 | N44030 | | gb: yy50c11.r1 Soares_multiple_sclerosis_{—} | 3.48E−06 | 2.02 |
| 422375 | AA768234 | Hs. 292430 | ESTs | 5.15E−04 | 2.02 |
| 419555 | AA244416 | | gb: nc07d11.s1 NCI_CGAP_Pr1 Homo sapiens | 1.34E−06 | 2.01 |
| 427840 | AI216654 | Hs. 98251 | ESTs | 5.62E−06 | 2.00 |
| 431249 | NM_004283 | Hs. 8036 | RAB3D, member RAS oncogene family | 2.90E−03 | 2.00 |
| 438573 | AW135084 | Hs. 127264 | ESTs, Weakly similar to JC5314 CDC28/cdc | 1.64E−05 | 1.98 |
| 451402 | AI793167 | | ESTs, Highly similar to AC004836 1 simil | 1.74E−04 | 1.98 |
| 447715 | AW291714 | Hs. 221703 | ESTs | 1.95E−07 | 1.98 |
| 428744 | BE267033 | Hs. 192853 | ubiquitin-conjugating enzyme E2G 2 (homo | 2.70E−04 | 1.98 |
| 414063 | H26904 | Hs. 75736 | apolipoprotein D | 1.83E−05 | 1.98 |
| 434646 | AF147445 | | gb: Homo sapiens full length insert cDNA | 3.61E−03 | 1.97 |
| 425036 | BE467912 | Hs. 154294 | discs, large (Drosophila) homolog 1 | 1.65E−03 | 1.97 |
| 452742 | AW589945 | Hs. 97876 | hypothetical protein DKFZp564K0322 | 1.71E−05 | 1.96 |
| 400663 | | | Target Exon | 9.40E−04 | 1.95 |
| 454328 | AW372097 | Hs. 278429 | hepatocellular carcinoma-associated anti | 5.19E−03 | 1.95 |
| 403686 | | | C4001366*: gi\|9837427\|gb\|AAG00570.1\|AF287 | 2.69E−04 | 1.94 |
| 402399 | | | C19001025*: gi\|11275298\|dbj\|BAB18302.1\|( | 5.65E−04 | 1.94 |
| 414584 | BE409585 | Hs. 117715 | hypothetical protein MGC10850 | 2.52E−05 | 1.94 |
| 409795 | AI934808 | | ESTs, Weakly similar to T46338 hypotheti | 6.48E−04 | 1.93 |
| 445123 | AI762911 | Hs. 145369 | ESTs | 3.97E−07 | 1.93 |
| 457561 | AA331517 | Hs. 286055 | chimerin (chimaerin) 2 | 7.32E−04 | 1.93 |
| 421511 | AA488940 | Hs. 105216 | hypothetical protein FLJ11125 | 4.63E−06 | 1.93 |
| 415167 | AA160784 | Hs. 26410 | ESTs | 9.17E−03 | 1.92 |
| 445403 | AI222423 | Hs. 308322 | ESTs | 8.85E−04 | 1.92 |
| 442793 | AI017798 | | ESTs, Weakly similar to TI47_HUMAN CARGO | 7.50E−08 | 1.91 |
| 401624 | | | Target Exon | 6.28E−07 | 1.91 |
| 413537 | BE146866 | | gb: QV4-HT0222-211099-014-f06 HT0222 Homo | 2.43E−05 | 1.90 |
| 444832 | AI807563 | Hs. 148452 | ESTs | 3.77E−03 | 1.88 |
| 455968 | BE168828 | | gb: QV1-HT0517-020400-145-f04 HT0517 Homo | 1.48E−05 | 1.88 |
| 401706 | | | Target Exon | 2.57E−04 | 1.87 |
| 416648 | R97488 | | gb: yq53b09.r1 Soares fetal liver spleen | 7.47E−05 | 1.87 |
| 458867 | AW995393 | | gb: QV0-BN0042-170300-163-g12 BN0042 Homo | 2.20E−07 | 1.86 |
| 405263 | | | ATPase, Na? transporting, alpha 2 () pol | 1.42E−04 | 1.86 |
| 457148 | AF091035 | Hs. 184627 | KIAA0118 protein | 8.35E−05 | 1.84 |
| 403796 | | | Target Exon | 2.31E−03 | 1.84 |
| 450657 | AK001579 | Hs. 25277 | hypothetical protein FLJ21065 | 3.73E−05 | 1.84 |
| 405639 | | | Target Exon | 4.60E−03 | 1.84 |
| 425526 | AA359933 | | gb: EST69040 Fetal lung II Homo sapiens c | 1.86E−03 | 1.84 |
| 407290 | AA633861 | Hs. 12106 | ESTs, Weakly similar to ALU7_HUMAN ALU S | 5.47E−04 | 1.83 |
| 448647 | AI702965 | Hs. 45005 | hypothetical protein FLJ12960 | 1.49E−04 | 1.83 |
| 448871 | BE616709 | Hs. 159265 | kruppel-related zinc finger protein hcKr | 7.64E−05 | 1.83 |
| 432018 | AA524447 | Hs. 152377 | ESTs | 2.23E−04 | 1.81 |
| 433545 | AA868510 | Hs. 112496 | ESTs | 5.50E−05 | 1.81 |
| 427789 | AA412428 | Hs. 48642 | hypothetical protein FLJ23093 | 2.54E−03 | 1.81 |
| 429013 | AJ012590 | Hs. 194728 | hexose-6-phosphate dehydrogenase (glucos | 2.54E−05 | 1.80 |
| 446887 | AI346656 | Hs. 156652 | hypothetical protein FLJ22800 | 7.83E−07 | 1.79 |
| 441718 | AW573183 | Hs. 127380 | ESTs | 3.62E−06 | 1.79 |
| 454287 | AF075292 | Hs. 49585 | fibroblast growth factor 18 | 2.82E−05 | 1.79 |
| 435125 | AA381895 | Hs. 284204 | F-box only protein 29 | 6.24E−07 | 1.79 |
| 408099 | AW151637 | | ESTs, Moderately similar to Z195_HUMAN Z | 1.70E−05 | 1.78 |
| 434884 | AF161340 | | Homo sapiens HSPC077 mRNA, partial cds | 3.95E−04 | 1.77 |
| 428303 | AW974476 | Hs. 183601 | regulator of G-protein signalling 16 | 2.82E−04 | 1.76 |
| 435028 | AW193035 | Hs. 187370 | ESTs | 2.89E−03 | 1.75 |
| 421472 | NM_003323 | Hs. 104636 | tubby like protein 2 | 1.28E−04 | 1.75 |
| 438687 | AA814084 | | ESTs | 5.89E−04 | 1.74 |
| 412004 | NM_005042 | Hs. 73952 | praline-rich protein Haelll subfamily 2 | 6.32E−05 | 1.74 |
| 403794 | | | Target Exon | 9.24E−05 | 1.74 |
| 441184 | AA922009 | | ESTs | 6.67E−07 | 1.73 |
| 430358 | AW604424 | Hs. 239891 | G protein-coupled receptor 35 | 1.21E−05 | 1.73 |
| 410312 | AW850953 | | gb: IL3-CT0220-150200-068-A11 CT0220 Homo | 4.73E−06 | 1.73 |
| 403772 | | | NM_003061: Homo sapiens slit (Drosophila) | 1.08E−04 | 1.73 |
| 405126 | | | Target Exon | 2.23E−04 | 1.72 |
| 438338 | NM_014861 | Hs. 6168 | KIAA0703 gene product | 4.80E−05 | 1.72 |
| 456782 | AK000462 | Hs. 132071 | ovarian carcinoma immunoreactive antigen | 8.79E−05 | 1.72 |
| 422097 | X61755 | Hs. 111473 | homeo box C5 | 6.28E−03 | 1.71 |
| 415593 | F13043 | Hs. 6918 | ESTs | 4.22E−03 | 1.71 |
| 409545 | BE296182 | Hs. 19002 | hypothetical protein MGC4675 | 8.81E−03 | 1.71 |
| 453855 | AA039576 | Hs. 37858 | ESTs, Weakly similar to ALUB_HUMAN !!!! | 5.82E−03 | 1.71 |
| 400729 | | | Target Exon | 3.52E−05 | 1.70 |
| 457505 | AL044659 | Hs. 43791 | ESTs | 6.22E−04 | 1.70 |
| 420548 | AA278246 | Hs. 920 | ESTs | 1.27E−03 | 1.69 |
| 433899 | AW628168 | Hs. 152684 | ESTs | 1.22E−03 | 1.69 |
| 423103 | AA322029 | | gb: EST24685 Cerebellum II Homo sapiens c | 7.95E−04 | 1.68 |
| 435367 | AI917684 | Hs. 85524 | for muscle specific ring finger 2 | 2.37E−03 | 1.68 |
| 447135 | T58148 | | gb: yb98g06.s1 Stratagene lung (937210) H | 2.13E−06 | 1.68 |
| 429642 | X68264 | Hs. 211579 | melanoma cell adhesion molecule (MCAM) ( | 1.65E−06 | 1.68 |
| 422189 | AF252292 | Hs. 112933 | Tax interaction protein 40 | 7.46E−03 | 1.68 |
| 422028 | AW090340 | Hs. 14337 | Homo sapiens cDNA FLJ14407 fis, clone HE | 3.17E−03 | 1.67 |
| 441777 | AI733440 | Hs. 127735 | ESTs | 8.00E−04 | 1.67 |
| 415897 | H08323 | Hs. 268712 | ESTs | 3.51E−04 | 1.67 |
| 403243 | | | Target Exon | 3.28E−05 | 1.66 |
| 402690 | | | Target Exon | 1.11E−05 | 1.66 |
| 445132 | Z44811 | Hs. 14928 | hypothetical protein FLJ12903 | 1.10E−05 | 1.66 |
| 405677 | | | Target Exon | 9.97E−03 | 1.66 |
| 441477 | BE379434 | Hs. 192127 | Homo sapiens, clone IMAGE: 3609326, mRNA, | 1.03E−03 | 1.66 |
| 400522 | | | Target Exon | 1.90E−06 | 1.66 |
| 407283 | T51008 | | gb: yb55e08.s1 Stratagene ovary (937217) | 7.26E−05 | 1.65 |
| 440509 | BE410132 | Hs. 134202 | ESTs, Weakly similar to T17279 hypotheti | 6.14E−06 | 1.65 |
| 453449 | W16752 | Hs. 32981 | sema domain, immunoglobulin domain (Ig), | 4.49E−06 | 1.65 |
| 431107 | AW451602 | Hs. 153590 | ESTs | 2.58E−06 | 1.65 |
| 410736 | AA205850 | Hs. 122823 | thousand and one amino acid protein kina | 2.55E−04 | 1.65 |
| 410255 | AA234006 | Hs. 321264 | LBP protein 32 | 1.07E−03 | 1.64 |
| 457394 | M86528 | Hs. 266902 | neurotrophin 5 (neurotrophin 4/5) | 1.37E−05 | 1.63 |
| 406140 | | | Target Exon | 7.83E−06 | 1.63 |
| 408932 | AW594172 | Hs. 278513 | TP53TG3 protein | 8.73E−03 | 1.63 |
| 453992 | AW014995 | Hs. 281080 | ESTs | 7.00E−06 | 1.62 |
| 418141 | AW845738 | Hs. 171118 | hypothetical protein FLJ00026 | 1.34E−04 | 1.62 |
| 454430 | AI082777 | Hs. 61384 | sema domain, seven thrombospondin repeat | 2.32E−04 | 1.61 |
| 439335 | AA742697 | Hs. 62492 | NM_052863: Homo sapiens secretoglobin, fa | 1.22E−05 | 1.61 |
| 433095 | AK001092 | Hs. 302480 | Homo sapiens cDNA FLJ10230 fis, clone HE | 4.03E−05 | 1.61 |
| 454527 | AW804626 | Hs. 151237 | hypothetical protein FLJ12526 | 7.10E−03 | 1.61 |
| 444462 | AA127530 | Hs. 286035 | Homo sapiens mRNA for KIAA1783 protein, | 6.17E−04 | 1.60 |
| 435724 | N39308 | Hs. 117898 | ESTs | 5.43E−08 | 1.60 |
| 411181 | AW820794 | Hs. 252406 | hypothetical protein FLJ12296 similar to | 1.80E−04 | 1.60 |
| 446705 | AW206634 | Hs. 224879 | ESTs | 3.59E−06 | 1.59 |
| 416249 | R85267 | Hs. 107692 | ESTs | 6.97E−04 | 1.59 |
| 421539 | AA292747 | Hs. 97296 | ESTs | 1.44E−04 | 1.59 |
| 423658 | AB003062 | Hs. 130760 | myosin phosphatase, target subunit 2 | 3.00E−04 | 1.59 |
| 426254 | BE018103 | | PP3111 protein | 1.45E−06 | 1.58 |
| 442674 | AI124736 | Hs. 142921 | ESTs | 1.19E−03 | 1.58 |
| 407845 | AL036518 | Hs. 118598 | Homo sapiens mRNA for KIAA1878 protein, | 2.96E−04 | 1.58 |
| 410626 | BE407727 | Hs. 199316 | gb: 601299771F1 NIH_MGC_21 Homo sapiens c | 1.10E−03 | 1.58 |
| 415160 | T82802 | | gb: yd38a04.r1 Soares fetal liver spleen | 3.67E−06 | 1.58 |
| 416397 | H53035 | | Homo sapiens AFG3L1 isoform 1 mRNA, part | 3.78E−03 | 1.58 |
| 401371 | | | ENSP00000198192*: BA438F9.1 (novel protei | 1.37E−04 | 1.58 |
| 457008 | AA410446 | Hs. 112011 | ESTs, Weakly similar to unknown [H.sapie | 7.30E−05 | 1.57 |
| 456093 | AL135220 | Hs. 77579 | apoptotic protease activating factor | 3.53E−04 | 1.57 |
| 408518 | BE162203 | Hs. 314758 | ESTs | 2.86E−03 | 1.57 |
| 406357 | | | Target Exon | 1.40E−03 | 1.56 |
| 401554 | | | Target Exon | 4.40E−03 | 1.56 |
| 423143 | M64676 | Hs. 124212 | potassium voltage-gated channel, Shaw-re | 6.15E−06 | 1.56 |
| 409911 | N43177 | Hs. 284217 | serologically defined colon cancer antig | 5.78E−03 | 1.56 |
| 445889 | BE465186 | Hs. 266958 | ESTs | 2.38E−05 | 1.55 |
| 405137 | | | Target Exon | 1.73E−03 | 1.55 |
| 418525 | AW450369 | Hs. 86937 | ESTs | 2.90E−04 | 1.55 |
| 425169 | AW292500 | Hs. 128514 | ESTs | 3.54E−03 | 1.55 |
| 404843 | | | C22000088*: gi\|6678165\|ref\|NP_033461.1\|s | 8.09E−04 | 1.55 |
| 456764 | AA325560 | Hs. 346401 | ESTs | 4.17E−04 | 1.55 |
| 412371 | AW946621 | | gb: RC2-ET0021-280400-011-e12 ET0021 Homo | 6.83E−03 | 1.55 |
| 458211 | AA922156 | Hs. 144751 | ESTs | 1.58E−03 | 1.55 |
| 403613 | | | C3000680*: gi\|5852484\|gb\|AAD54072.1\|AF131 | 2.05E−03 | 1.54 |
| 402534 | | | Target Exon | 2.23E−04 | 1.54 |
| 428189 | AA424030 | Hs. 46627 | ESTs | 2.07E−06 | 1.54 |
| 401629 | | | ENSP00000251306*: HYPOTHETICAL 42.6 kDa P | 1.46E−05 | 1.54 |
| 426449 | AL134009 | Hs. 169936 | Homo sapiens mRNA; cDNA DKFZp586N1918 (f | 4.75E−03 | 1.53 |
| 404731 | | | NM_006180*: Homo sapiens neurotrophic tyr | 3.51E−03 | 1.53 |
| 422111 | W04245 | Hs. 107761 | ESTs, Weakly similar to unnamed protein | 4.17E−04 | 1.53 |
| 451461 | AI968438 | Hs. 265349 | ESTs | 4.55E−03 | 1.53 |
| 450235 | AA007512 | Hs. 17538 | ESTs | 3.39E−03 | 1.52 |
| 416323 | N72630 | | Homo sapiens genomic DNA, chromosome 21q | 1.17E−05 | 1.52 |
| 438808 | M73980 | Hs. 129053 | Homo sapiens NOTCH 1 (N1) mRNA, complete | 3.12E−07 | 1.52 |
| 452655 | R85491 | Hs. 33466 | ESTs, Weakly similar to DPOZ_HUMAN DNA P | 9.50E−04 | 1.52 |
| 431777 | AA570296 | Hs. 105470 | found in inflammatory zone 1 | 4.41E−05 | 1.52 |
| 423086 | AB028984 | Hs. 123420 | KIAA1061 protein | 2.50E−06 | 1.51 |
| 407173 | T64349 | | gb: yc10d08.s1 Stratagene lung (937210) H | 2.11E−04 | 1.51 |
| 449482 | AI784266 | Hs. 28774 | ESTs, Weakly similar to I38022 hypotheti | 1.71E−03 | 1.51 |
| 404219 | | | NM_004557*: Homo sapiens Notch (Drosophil | 1.02E−05 | 1.51 |
| 441493 | AW070446 | Hs. 127037 | ESTs | 6.77E−04 | 1.51 |
| 405913 | | | NM_004349: Homo sapiens core-binding fact | 2.61E−03 | 1.50 |
| 458223 | AW297673 | Hs. 190526 | ESTs | 6.98E−04 | 1.50 |
| 452854 | AA437061 | Hs. 14060 | prokineticin 1 precursor | 1.67E−06 | 1.50 |
| 445735 | AW503939 | Hs. 107149 | novel protein similar to archaeal, yeast | 1.23E−03 | 1.50 |
| 446052 | AA358760 | Hs. 95893 | gb: EST67699 Fetal lung II Homo sapiens c | 1.00E−06 | 1.50 |
| 407124 | R08160 | | gb: yf18a07.s1 Soares fetal liver spleen | 8.86E−03 | 1.49 |
| 458968 | AI933128 | Hs. 25220 | like-glycosyltransferase | 1.73E−03 | 1.49 |
| 408937 | AA210734 | Hs. 291386 | ESTs | 8.62E−03 | 1.49 |
| 403180 | | | Target Exon | 8.69E−05 | 1.49 |
| 432202 | BE301302 | Hs. 7200 | ESTs, Highly similar to AC007055 5 unkno | 4.82E−03 | 1.49 |
| 407654 | AW064121 | Hs. 279175 | ESTs | 1.06E−03 | 1.48 |
| 437408 | AL359598 | Hs. 36606 | Homo sapiens mRNA; cDNA DKFZp547B086 (fr | 7.68E−05 | 1.48 |
| 443495 | N95833 | Hs. 80395 | ESTs | 4.96E−05 | 1.48 |
| 436666 | AI191588 | Hs. 1179 | TATA box binding protein (TBP)-associate | 6.61E−03 | 1.48 |
| 426330 | M77235 | Hs. 169331 | sodium channel, voltage-gated, type V, a | 3.38E−04 | 1.48 |
| 446066 | AI343931 | Hs. 149383 | ESTs | 7.17E−05 | 1.48 |
| 422754 | AA316476 | Hs. 171811 | adenylate kinase 2 | 1.62E−03 | 1.48 |
| 401595 | | | Rag C protein | 3.73E−05 | 1.48 |
| 406089 | | | Target Exon | 4.83E−03 | 1.48 |
| 401899 | | | Target Exon | 8.77E−05 | 1.47 |
| 412433 | AA102400 | | ESTs | 7.09E−05 | 1.47 |
| 402137 | | | Target Exon | 2.63E−03 | 1.46 |
| 404701 | | | NM_022779: Homo sapiens hypothetical prot | 2.50E−03 | 1.46 |
| 455663 | BE065679 | | gb: RC3-BT0316-270400-016-e10 BT0316 Homo | 7.55E−04 | 1.46 |
| 407032 | U73799 | | gb: Human dynactin mRNA, partial cds. | 1.67E−08 | 1.46 |
| 449904 | AW304472 | Hs. 200088 | ESTs | 1.91E−03 | 1.46 |
| 403474 | | | NM_030968: Homo sapiens G protein coupled | 2.90E−05 | 1.45 |
| 438991 | W78940 | Hs. 20526 | ESTs | 6.38E−03 | 1.45 |
| 443503 | AV645438 | Hs. 282927 | ESTs | 7.43E−03 | 1.45 |
| 453224 | AI989700 | Hs. 232320 | ESTs | 4.46E−05 | 1.45 |
| 452263 | AA025116 | Hs. 33333 | ESTs | 1.96E−05 | 1.45 |
| 405762 | | | CX000374*: gi\|11433304\|ref\|XP_006583.1\|h | 2.15E−04 | 1.45 |
| 427626 | AI364292 | Hs. 283696 | ESTs, Weakly similar to A43932 mucin 2 p | 5.41E−03 | 1.45 |
| 417741 | R12418 | Hs. 274728 | ESTs | 1.12E−03 | 1.45 |
| 438526 | AI090671 | Hs. 134807 | hypothetical protein FLJ12057 | 7.48E−03 | 1.45 |
| 438173 | AK001271 | Hs. 6092 | f-box and leucine-rich repeat protein 2 | 2.09E−03 | 1.44 |
| 458826 | AI918672 | Hs. 213783 | ESTs | 1.92E−05 | 1.44 |
| 415255 | R39947 | Hs. 7237 | ESTs | 8.01E−03 | 1.44 |
| 422091 | AI906339 | Hs. 97927 | ESTs | 9.01E−03 | 1.44 |
| 433010 | AW970018 | | gb: EST382097 MAGE resequences, MAGK Homo | 2.64E−03 | 1.44 |
| 422800 | AW953176 | | Homo sapiens cDNA: FLJ23260 fis, clone C | 1.03E−03 | 1.44 |
| 439070 | AI733278 | Hs. 7621 | ESTs | 1.64E−03 | 1.44 |
| 405590 | | | CX001497*: qi\|4557543\|ref\|NP_001384.1\| ex | 1.91E−06 | 1.43 |
| 414648 | H54381 | | gb: yq89a03.s1 Soares fetal liver spleen | 1.07E−05 | 1.43 |
| 409319 | AW752736 | Hs. 33565 | ESTs | 6.79E−03 | 1.43 |
| 426065 | N32049 | Hs. 29423 | gb: yw96g08.s1 Soares_placenta_8to9weeks_{—} | 2.67E−04 | 1.43 |
| 445771 | AI804325 | Hs. 201060 | ESTs | 5.21E−04 | 1.43 |
| 418642 | AA226049 | Hs. 297915 | ESTs | 2.51E−05 | 1.42 |
| 429521 | BE048708 | Hs. 50949 | ESTs | 3.99E−05 | 1.42 |
| 414262 | AW975616 | Hs. 291469 | ESTs | 1.22E−04 | 1.42 |
| 437093 | AA744334 | | gb: ny59b06.s1 NCI_CGAP_Pr18 Homo sapiens | 1.78E−04 | 1.42 |
| 421093 | AA906434 | Hs. 3776 | zinc finger protein 216 | 4.09E−05 | 1.42 |
| 404825 | | | C22000161*: gi\|2443331\|dbj\|BAA22375.1\|(D | 5.52E−03 | 1.42 |
| 408768 | AW663111 | Hs. 94958 | tubulin tyrosine ligase-like 1 | 8.41E−04 | 1.41 |
| 402638 | | | guanine nucleotide binding protein (G pr | 1.03E−03 | 1.41 |
| 406458 | | | C14000133*: gi\|1082739\|pir\|C44324 protea | 2.14E−06 | 1.41 |
| 414327 | BE408145 | Hs. 185254 | ESTs, Weakly similar to T24435 hypotheti | 2.01E−05 | 1.41 |
| 435026 | AA744812 | Hs. 177135 | ESTs | 1.80E−03 | 1.41 |
| 445481 | AW661846 | | ESTs | 3.72E−05 | 1.40 |
| 410922 | AW810845 | | gb: MR2-ST0129-221099-013-a03 ST0129 Homo | 3.02E−03 | 1.40 |
| 412044 | BE086167 | | gb: PM3-BT0673-260200-001-d01 BT0673 Homo | 2.88E−08 | 1.40 |
| 457994 | AW136239 | Hs. 132922 | ESTs, Weakly similar to TI47_HUMAN CARGO | 4.12E−07 | 1.40 |
| 448464 | AI522053 | | ESTs | 1.16E−03 | 1.40 |
| 442340 | AW882532 | Hs. 8257 | cytokine inducible SH2-containing protei | 3.53E−04 | 1.39 |
| 428887 | AA437009 | Hs. 98984 | ESTs | 6.17E−09 | 1.39 |
| 436558 | AI360238 | Hs. 157730 | ESTs | 3.23E−04 | 1.39 |
| 428406 | BE279930 | Hs. 98564 | hypothetical protein DKFZp434G0920 | 1.31E−03 | 1.39 |
| 427267 | AI201185 | Hs. 119164 | ESTs | 9.05E−04 | 1.39 |
| 436549 | AJ297365 | Hs. 334478 | hypothetical protein MPMGp800M05499Q3 | 1.76E−04 | 1.38 |
| 404923 | | | NM_020131*: Homo sapiens ataxin-1 ubiquit | 3.88E−05 | 1.38 |
| 403209 | | | Target Exon | 3.66E−03 | 1.38 |
| 432909 | AA570111 | | ESTs, Weakly similar to ALUE_HUMAN!!!! | 8.59E−04 | 1.38 |
| 459304 | AW005809 | Hs. 158291 | ESTs, Weakly similar to CHD4_HUMAN CHROM | 5.40E−03 | 1.38 |
| 415597 | Z44384 | | gb: HSC1ZC041 normalized infant brain cDN | 5.65E−05 | 1.38 |
| 458544 | AI631036 | Hs. 196843 | ESTs | 4.62E−04 | 1.37 |
| 440075 | BE268806 | | gb: 601139917F1 NIH_MGC_8 Homo sapiens cD | 2.27E−04 | 1.37 |
| 441194 | BE274581 | | ESTs, Moderately similar to S65657 alpha | 6.64E−03 | 1.37 |
| 457451 | H06075 | Hs. 270232 | ESTs | 6.68E−06 | 1.37 |
| 400635 | | | C10000818*: gi\|7661882\|ref\|NP_055697.1\|K | 2.66E−06 | 1.37 |
| 432092 | AF135026 | | kallikrein 9 | 1.01E−03 | 1.36 |
| 419042 | T81429 | Hs. 221065 | ESTs | 1.20E−03 | 1.36 |
| 454537 | BE391493 | | Human DNA sequence from clone RP5-852M4 | 9.75E−03 | 1.36 |
| 446403 | AW452904 | | gb: UI-H-BI3-aly-h-11-0-UI.s1 NCI_CGAP_Su | 8.47E−04 | 1.36 |
| 402349 | | | Target Exon | 4.33E−05 | 1.36 |
| 442907 | AI023763 | Hs. 79707 | ESTs | 1.21E−03 | 1.36 |
| 436394 | AA531187 | Hs. 126705 | ESTs | 5.26E−09 | 1.36 |
| 440745 | AW303627 | Hs. 143301 | ESTs | 1.69E−04 | 1.36 |
| 441662 | AI807406 | | ESTs | 3.93E−03 | 1.36 |
| 427951 | AI826125 | Hs. 43546 | ESTs | 3.49E−03 | 1.36 |
| 458354 | AI024181 | Hs. 131657 | ESTs | 3.18E−03 | 1.36 |
| 414871 | BE549179 | Hs. 29008 | gb: 601078714F1 NIH_MGC_12 Homo sapiens c | 7.53E−04 | 1.36 |
| 454930 | AW845987 | Hs. 68864 | ESTs, Weakly similar to phosphatidylseri | 1.57E−03 | 1.36 |
| 456922 | AA477435 | Hs. 193669 | hypothetical protein DKFZp586J1119 | 1.19E−03 | 1.35 |
| 453356 | BE539237 | Hs. 182698 | mitochondrial ribosomal protein L20 | 8.58E−03 | 1.35 |
| 411154 | AW819848 | | gb: QV0-ST0294-070300-151-b04 ST0294 Homo | 8.20E−04 | 1.35 |
| 442558 | AI000792 | Hs. 108209 | ESTs | 2.62E−04 | 1.35 |
| 459002 | AI915933 | Hs. 102647 | ESTs, Weakly similar to WASP interacting | 7.83E−05 | 1.35 |
| 452236 | AI130858 | Hs. 143218 | ESTs | 2.66E−03 | 1.35 |
| 456110 | AW872878 | Hs. 289072 | hypothetical protein FLJ22175 | 9.25E−03 | 1.35 |
| 421324 | BE257515 | Hs. 103503 | deoxyribonuclease I-like 2 | 9.35E−05 | 1.34 |
| 414614 | BE390050 | Hs. 48642 | hypothetical protein FLJ23093 | 5.55E−03 | 1.34 |
| 448544 | AI535819 | | gb: jun1.P1.G1 conorm Homo sapiens cDNA 3 | 9.28E−04 | 1.34 |
| 458575 | AW362488 | Hs. 12797 | DEAD/H (Asp-Glu-Ala-Asp/His) box polypep | 9.57E−05 | 1.34 |
| 417597 | BE297053 | Hs. 347310 | hypothetical protein FLJ14627 | 1.63E−04 | 1.34 |
| 430725 | AA485056 | | ESTs | 3.62E−03 | 1.34 |
| 432190 | T80206 | Hs. 14716 | ESTs | 6.52E−04 | 1.34 |
| 445690 | AW675572 | Hs. 193620 | ESTs | 2.53E−03 | 1.34 |
| 420813 | X51501 | Hs. 99949 | prolactin-induced protein | 2.38E−03 | 1.34 |
| 422579 | BE407266 | | gb: 601301004F1 NIH_MGC_21 Homo sapiens c | 3.32E−03 | 1.34 |
| 402951 | | | ENSP00000243967*: DJ1100H13.4 (putative R | 4.06E−04 | 1.34 |
| 436516 | AW134701 | Hs. 245358 | ESTs | 1.00E−03 | 1.34 |
| 424334 | AA393460 | Hs. 13386 | gb: zt71e05.r1 Soares_testis_NHT Homo sap | 1.50E−05 | 1.33 |
| 432081 | AW972788 | | gb: EST384883 MAGE resequences, MAGL Homo | 9.35E−04 | 1.33 |
| 458306 | AF039185 | Hs. 41974 | Homo sapiens, clone IMAGE: 4100953, mRNA | 1.53E−03 | 1.33 |
| 421228 | AA811598 | Hs. 275809 | ESTs | 1.31E−04 | 1.33 |
| 402447 | | | C1000201: gi\|204416\|gb\|AAA02627.1\|(L0519 | 6.43E−05 | 1.33 |
| 403545 | | | Target Exon | 5.81E−06 | 1.33 |
| 408376 | AW971303 | Hs. 241869 | ESTs | 7.57E−03 | 1.33 |
| 420693 | NM_001972 | Hs. 99863 | elastase 2. neutrophil | 6.28E−04 | 1.33 |
| 426157 | AA370977 | Hs. 345728 | STAT induced STAT inhibitor 3 | 1.99E−10 | 1.33 |
| 453196 | AW003567 | | ESTs | 1.98E−04 | 1.32 |
| 421798 | N74880 | | N-acylsphingosine amidohydrolase (acid c | 4.05E−03 | 1.32 |
| 458077 | AA100768 | Hs. 48485 | ESTs, Weakly similar to I38022 hypotheti | 1.60E−03 | 1.32 |
| 400343 | AB045830 | Hs. 160953 | Target | 3.15E−04 | 1.32 |
| 402561 | | | Target Exon | 2.63E−04 | 1.32 |
| 454803 | AW860148 | | gb: RC0-CT0379-290100-032-b10 CT0379 Homo | 4.50E−05 | 1.32 |
| 413619 | BE153524 | | gb: PM0-HT0339-241199-002-C03 HT0339 Homo | 2.33E−06 | 1.31 |
| 446749 | NM_016069 | Hs. 16089 | CGI-136 protein | 2.11E−05 | 1.31 |
| 447680 | AW768676 | Hs. 302022 | PR domain containing 16 | 1.19E−03 | 1.31 |
| 425012 | T77666 | Hs. 92414 | Homo sapiens cDNA: FLJ22030 fis, clone H | 6.29E−03 | 1.31 |
| 416675 | H73802 | Hs. 35381 | ESTs | 1.60E−03 | 1.31 |
| 437568 | AI954795 | Hs. 156135 | ESTs | 4.02E−03 | 1.30 |
| 401516 | | | C3001641*: gi\|11359861\|pir\|\|JC7326 bood P | 1.62E−03 | 1.30 |
| 428177 | AA423967 | | ESTs | 1.80E−03 | 1.30 |
| 452320 | AA042873 | Hs. 160412 | ESTs | 1.14E−03 | 1.30 |
| 442496 | R55073 | Hs. 76780 | ESTs | 6.05E−07 | 1.30 |
| 405691 | | | NM_013439*: Homo sapiens paired immunoglo | 1.56E−06 | 1.30 |
| 409540 | AW409569 | Hs. 101550 | gb: fh01e09.x1 NIH_MGC_17 Homo sapiens cD | 7.89E−05 | 1.30 |
| 411288 | AW835511 | | gb: QV0-LT0015-180200-127-d02 LT0015 Homo | 1.34E−03 | 1.30 |
| 418551 | T75517 | | gb: yd63c01.r1 Soares fetal liver spleen | 8.37E−03 | 1.30 |
| 415449 | H15034 | | gb: ym20a03.s1 Soares infant brain 1NIB H | 1.60E−03 | 1.29 |
| 457677 | AA628890 | | ESTs | 1.76E−03 | 1.29 |
| 423310 | AA325225 | Hs. 124023 | Homo sapiens cDNA FLJ14218 fis, clone NT | 1.17E−03 | 1.29 |
| 420800 | AI041991 | Hs. 132227 | Homo sapiens, clone MGC: 13138, mRNA, com | 2.29E−03 | 1.29 |
| 438292 | AI139006 | Hs. 122352 | ESTs | 1.97E−03 | 1.29 |
| 412429 | AV650262 | Hs. 75765 | GRO2 oncogene | 5.81E−08 | 1.29 |
| 446528 | AU076640 | Hs. 15243 | nucleolar protein 1 (120 kD) | 1.99E−04 | 1.28 |
| 454908 | AW840458 | | gb: CM3-LT0079-180200-096<01 LT0079 Homo | 1.91E−03 | 1.28 |
| 423505 | AF064090 | Hs. 129708 | tumor necrosis factor (ligand) superfami | 9.02E−04 | 1.28 |
| 404126 | | | ENSP00000211797*: Helicase SKI2W (Helicas | 2.12E−03 | 1.28 |
| 432749 | NM_014438 | Hs. 278909 | interleukin 1, eta | 1.09E−03 | 1.28 |
| 450675 | AA010662 | Hs. 188639 | ESTs | 4.57E−04 | 1.28 |
| 404046 | | | NM_019120*: Homo sapiens protocadherin be | 9.68E−05 | 1.28 |
| 402991 | | | Target Exon | 3.40E−04 | 1.28 |
| 452165 | R17489 | Hs. 28264 | Homo sapiens mRNA; cDNA DKFZp564L0822 (f | 2.07E−04 | 1.28 |
| 445268 | AI218358 | Hs. 175048 | ESTs | 4.78E−03 | 1.28 |
| 433664 | AW292176 | Hs. 245834 | ESTs | 8.30E−03 | 1.27 |
| 416348 | H65887 | Hs. 272163 | ESTs | 1.09E−03 | 1.27 |
| 442705 | AI264634 | Hs. 131127 | ESTs | 5.94E−04 | 1.27 |
| 440625 | BE539853 | Hs. 22452 | Homo sapiens mRNA for KIAA1737 protein, | 3.66E−04 | 1.27 |
| 420459 | AF016045 | Hs. 97905 | ovo (Drosophila) homolog-like 1 | 6.83E−03 | 1.27 |
| 413737 | BE160704 | | gb: PM1-HT0422-170100-005-b07 HT0422 Homo | 5.31E−06 | 1.27 |
| 408183 | AW968065 | Hs. 44143 | polybromo 1 | 1.93E−03 | 1.27 |
| 420380 | AA640891 | Hs. 102406 | ESTs | 4.61E−05 | 1.27 |
| 454186 | BE141030 | | gb: MR0-HT0067-201099-002-h11 HT0067 Homo | 2.63E−03 | 1.27 |
| 438692 | AB007950 | Hs. 6360 | KIAA0481 gene product | 5.51E−04 | 1.27 |
| 402320 | | | f-box and leucine-rich repeat protein 7 | 6.75E−06 | 1.27 |
| 414189 | BE378841 | Hs. 73452 | hypothetical protein MGC10791 | 3.64E−05 | 1.26 |
| 413961 | BE207098 | | gb: ba10e09.y1 NIH_MGC_7 Homo sapiens cDN | 5.19E−03 | 1.26 |
| 454598 | AW809716 | | gb: MR4-ST0124-241199-026-h09 ST0124 Homo | 1.50E−04 | 1.26 |
| 408935 | BE539706 | Hs. 285363 | ESTs | 7.92E−04 | 1.26 |
| 450653 | AI191771 | Hs. 29764 | wingless-type MMTV integration site fami | 4.58E−03 | 1.26 |
| 403227 | | | Target Exon | 1.60E−03 | 1.26 |
| 439471 | W69839 | Hs. 58033 | ESTs | 3.83E−03 | 1.26 |
| 433782 | AF090945 | Hs. 306564 | Homo sapiens clone HQ0670 | 8.19E−05 | 1.26 |
| 425446 | U34802 | Hs. 157433 | gap junction protein, alpha 8,50 kD (con | 6.39E−07 | 1.26 |
| 417313 | AA195602 | | gb: zr32f09.r1 Soares_NhHMPu_S1 Homo sapi | 3.92E−05 | 1.26 |
| 437180 | BE180234 | Hs. 281462 | Homo sapiens cDNA FLJ14793 fis, clone NT | 7.39E−04 | 1.25 |
| 436419 | AI948626 | Hs. 171356 | ESTs | 1.91E−03 | 1.25 |
| 424713 | AI660243 | Hs. 318545 | transmembrane protease, serine 2 | 7.11E−03 | 1.25 |
| 401265 | | | C18000090*: gi\|6678656\|ref\|NP_032506.1\|1 | 3.20E−03 | 1.25 |
| 426212 | S71824 | Hs. 167988 | neural cell adhesion molecule 1 | 5.71E−05 | 1.25 |
| 410285 | AA083609 | | gb: zm63d05.r1 Stratagene fibroblast (937 | 7.72E−04 | 1.25 |
| 408432 | AW195262 | | gb: xn67b05.x1 NCI_CGAP_CML1 Homo sapiens | 6.30E−04 | 1.25 |
| 449405 | AA001350 | | gb: zh83h05.r1 Soares_fetal_liver_spleen_{—} | 6.58E−03 | 1.25 |
| 431357 | T81154 | | gb: yd24f01.r1 Soares fetal liver spleen | 4.61E−03 | 1.24 |
| 459012 | AA047221 | Hs. 59752 | ESTs | 7.13E−03 | 1.24 |
| 408725 | AA131539 | Hs. 15669 | ESTs | 4.13E−04 | 1.24 |
| 428568 | AC004755 | | Homo sapiens chromosome 19, fosmid 37502 | 2.08E−04 | 1.24 |
| 453492 | AA235214 | Hs. 184793 | DKFZP434F195 protein | 2.09E−03 | 1.24 |
| 404247 | | | NM_000500*: Homo sapiens cytochrome P450, | 3.47E−03 | 1.24 |
| 427595 | NM_016153 | Hs. 179756 | LW-1 | 2.01E−03 | 1.24 |
| 408038 | AW138673 | Hs. 252928 | ESTs | 1.35E−05 | 1.24 |
| 440353 | AI366674 | Hs. 286194 | hypothetical protein FLJ22233 | 8.84E−03 | 1.24 |
| 438649 | AA813136 | Hs. 143014 | ESTs | 1.75E−03 | 1.24 |
| 406370 | | | interleukin 11 | 1.24E−04 | 1.24 |
| 415037 | AA159478 | | ESTs | 2.21E−04 | 1.24 |
| 451005 | AI751946 | Hs. 20017 | chromosome 22 open reading frame 4 | 3.85E−03 | 1.24 |
| 404075 | | | Target Exon | 3.41E−03 | 1.24 |
| 401703 | | | NM_001053: Homo sapiens somatostatin rece | 1.24E−04 | 1.23 |
| 402252 | | | Target Exon | 4.93E−06 | 1.23 |
| 412538 | T31119 | Hs. 7177 | ESTs | 7.66E−04 | 1.23 |
| 440190 | AW752597 | | gb: IL3-CT0214-161299-045-B06 CT0214 Homo | 3.52E−03 | 1.23 |
| 402316 | | | NM_013447: Homo sapiens egf-like module c | 3.46E−03 | 1.23 |
| 437913 | AI140825 | Hs. 121623 | ESTs | 9.11E−04 | 1.23 |
| 458146 | AI286094 | | ESTs | 9.62E−05 | 1.23 |
| 452218 | AI936010 | Hs. 17649 | ESTs | 3.58E−04 | 1.23 |
| 437057 | BE548498 | Hs. 84529 | ESTs, Weakly similar to Z202_HUMAN ZINC | 3.02E−03 | 1.22 |
| 400818 | | | Target Exon | 1.76E−05 | 1.22 |
| 409749 | AW977045 | Hs. 185981 | ESTs, Weakly similar to gag [H. sapiens] | 1.55E−05 | 1.22 |
| 422626 | AA344932 | Hs. 118786 | metallothionein 2A | 4.89E−04 | 1.22 |
| 416666 | H73028 | Hs. 268992 | ESTs | 1.34E−05 | 1.21 |
| 409975 | AW839324 | | gb: CM0-LT0069-210100-157-h04 LT0069 Homo | 1.78E−03 | 1.21 |
| 404900 | | | NM_025213: Homo sapiens spectrin, beta, n | 8.14E−03 | 1.21 |
| 416369 | AW160617 | Hs. 24884 | hypothetical protein FLJ13390 similar to | 1.83E−04 | 1.21 |
| 417761 | R13727 | Hs. 21435 | ESTs | 2.43E−04 | 1.21 |
| 419299 | AI311085 | | hypothetical protein FLJ22573 | 2.01E−04 | 1.21 |
| 400877 | | | cofilin 1 (non-muscle) | 8.03E−03 | 1.21 |
| 437032 | AW867372 | Hs. 153261 | immunoglobulin heavy constant mu | 1.61E−03 | 1.21 |
| 423358 | AI815474 | Hs. 343866 | gb: au47f10.y1 Schneider fetal brain 0000 | 1.38E−04 | 1.21 |
| 400111 | | | Eos Control | 3.47E−03 | 1.21 |
| 402610 | | | Target Exon | 4.97E−04 | 1.21 |
| 404769 | | | NM_007037*: Homo sapiens a disintegrin-li | 8.45E−05 | 1.20 |
| 416694 | AW161284 | Hs. 79564 | neuronal PAS domain protein 1 | 4.72E−03 | 1.20 |
| 433182 | AB039920 | Hs. 127821 | BWRT protein | 5.55E−07 | 1.20 |
| 453899 | AW134536 | Hs. 243994 | ESTs | 2.91E−03 | 1.20 |
| 447563 | BE536115 | Hs. 160983 | EST | 1.53E−04 | 1.20 |
| 423966 | AF233523 | Hs. 136506 | spectrin, beta, non-erythrocytic 5 | 2.87E−05 | 1.20 |
| 447224 | BE617125 | Hs. 142076 | gb: 601441664F1 NIH_MGC_65 Homo sapiens c | 4.73E−05 | 1.20 |
| 456618 | BE245649 | Hs. 345728 | STAT induced STAT inhibitor 3 | 3.29E−05 | 1.20 |
| 432345 | AW780191 | Hs. 6523 | chromosome 1 open reading frame 12 | 6.34E−03 | 1.20 |
| 458281 | AW661820 | Hs. 44131 | ESTs | 1.36E−06 | 1.19 |
| 409280 | AW372614 | Hs. 82962 | rTS beta protein | 1.51E−04 | 1.19 |
| 436375 | AJ227870 | | gb: Homo sapiens partial mRNA; ID YG81-1 | 6.60E−03 | 1.19 |
| 423308 | AI365680 | Hs. 114085 | Homo sapiens mRNA for KIAA1755 protein. | 6.34E−05 | 1.19 |
| 417213 | BE257508 | Hs. 24719 | modulator of apoptosis 1 | 7.70E−05 | 1.19 |
| 427983 | M17706 | Hs. 2233 | colony stimulating factor 3 (granulocyte | 5.82E−06 | 1.18 |
| 409818 | AW500969 | | gb: UI-HF-BP0p-aiy-c-07-0-Ul.r1 NIH_MGC_5 | 7.58E−03 | 1.18 |
| 453741 | AW855522 | | gb: CM4-CT0275-040500-521-c06 CT0275 Homo | 1.98E−04 | 1.18 |
| 447455 | H38335 | Hs. 6750 | Homo sapiens mRNA for FLJ00058 protein, | 1.35E−03 | 1.18 |
| 402863 | | | C1000493*: gi\|9506977\|ref\|NP_062075.1\| ph | 3.85E−03 | 1.18 |
| 439665 | W93979 | | gb: zd97h10.r1 Soares_fetal_heart_NbHH19W | 2.71E−03 | 1.18 |
| 453198 | AI470361 | Hs. 61684 | ESTs, Weakly similar to T17307 hypotheti | 4.42E−06 | 1.18 |
| 458025 | AI275406 | Hs. 32450 | gb: ql63c10.x1 Soares_NhHMPu_S1 Homo sapi | 1.60E−05 | 1.18 |
| 439547 | BE000242 | Hs. 192068 | ESTs | 5.54E−03 | 1.17 |
| 429947 | AA461163 | Hs. 270391 | ESTs, Moderately similar to ALU1_HUMAN A | 3.37E−03 | 1.17 |
| 455977 | BE176477 | | gb: RC3-HT0585-160300-022-b01 HT0585 Homo | 2.00E−04 | 1.17 |
| 457424 | AW876082 | | gb: CM4-PT0015-071299-057-e05 PT0015 Homo | 8.04E−04 | 1.17 |
| 430204 | AA618335 | | ESTs, Weakly similar to YKQ3_CAEEL HYPOT | 2.57E−03 | 1.17 |
| 450335 | BE218355 | Hs. 201781 | ESTs, Weakly similar to B34087 hypotheti | 1.65E−04 | 1.17 |
| 458237 | AI741225 | Hs. 20924 | ESTs, Weakly similar to neogenin [H.sapi | 5.20E−08 | 1.17 |
| 444295 | AI201301 | Hs. 143837 | ESTs | 5.95E−03 | 1.17 |
| 441860 | AW451330 | Hs. 348198 | hypothetical protein FLJ20262 | 6.20E−07 | 1.17 |
| 435218 | T84045 | Hs. 119321 | ESTs | 2.19E−03 | 1.17 |
| 430885 | U60181 | Hs. 248115 | growth hormone secretagogue receptor | 6.04E−07 | 1.17 |
| 402012 | | | ubiquitin specific protease 15 | 2.10E−04 | 1.17 |
| 457204 | BE264152 | Hs. 221994 | ESTs | 4.15E−04 | 1.17 |
| 458095 | AI375740 | Hs. 54580 | ring finger protein 27 | 5.49E−03 | 1.17 |
| 455638 | BE063665 | | gb: RC0-BT0291-031199-031-d08 BT0291 Homo | 9.13E−05 | 1.16 |
| 450799 | AW407504 | | gb: UI-HF-BM0-adk-g-12-0-Ul.r1 NIH_MGC_38 | 2.38E−04 | 1.16 |
| 430326 | BE251590 | Hs. 239370 | DKFZP727I051 protein | 2.52E−03 | 1.16 |
| 431256 | NM_006093 | Hs. 251386 | proteoglycan 3 | 2.66E−05 | 1.16 |
| 458857 | AI627342 | Hs. 224601 | ESTs | 3.20E−03 | 1.16 |
| 412944 | AA384110 | Hs. 197143 | ESTs | 4.33E−03 | 1.16 |
| 449721 | AW073727 | Hs. 210265 | ESTs | 1.02E−04 | 1.16 |
| 452773 | AW138920 | Hs. 342009 | ESTs | 8.96E−03 | 1.16 |
| 431972 | AI805145 | Hs. 191711 | ESTs, Weakly similar to T2D3_HUMAN TRANS | 1.12E−04 | 1.16 |
| 402411 | | | C1002484*: gi\|12052832\|emb\|CAB66589.1\| (A | 2.35E−03 | 1.16 |
| 421432 | AL049553 | Hs. 104311 | novel protein with MAM domain | 1.40E−03 | 1.15 |
| 444207 | AI565004 | | cathepsin D (lysosomal aspartyl protease | 2.97E−07 | 1.15 |
| 423266 | AA323875 | Hs. 193574 | ESTs | 5.04E−03 | 1.15 |
| 455194 | AW947513 | | gb: RC0-MT0002-140300-011-e04 MT0002 Homo | 3.49E−05 | 1.15 |
| 440054 | AW661947 | Hs. 6891 | splicing factor, arginine/serine-rich 6 | 4.89E−05 | 1.15 |
| 403567 | | | Target Exon | 7.42E−03 | 1.15 |
| 413309 | BE312608 | | gb: 601151425F1 NIH_MGC_19 Homo sapiens c | 1.75E−06 | 1.15 |
| 457140 | AI279960 | Hs. 178140 | ESTs | 7.01E−04 | 1.15 |
| 458526 | AI202885 | Hs. 148611 | ESTs | 1.35E−03 | 1.15 |
| 430991 | AW968826 | Hs. 106432 | Homo sapiens cDNA FLJ13410 fis, clone PL | 3.98E−06 | 1.15 |
| 401831 | | | C17000644*: gi\|631600\|pir\|\|S47094 hypothe | 5.23E−03 | 1.15 |
| 403248 | | | ESTs, Weakly similar to I78885 serine/th | 5.87E−04 | 1.15 |
| 419909 | AL136653 | Hs. 93675 | decidual protein induced by progesterone | 8.17E−05 | 1.14 |
| 444552 | AW295211 | Hs. 230777 | ESTs | 2.00E−03 | 1.14 |
| 441580 | AA938999 | Hs. 271414 | ESTs, Moderately similar to ALU7_HUMAN A | 2.38E−05 | 1.14 |
| 402190 | | | C19000835*: gi\|10946730\|ref\|NP_067362.1\| | 6.88E−06 | 1.14 |
| 414156 | BE392588 | Hs. 75777 | transgelin | 5.43E−03 | 1.14 |
| 416007 | N68218 | Hs. 105998 | ESTs | 1.13E−03 | 1.14 |
| 455291 | S72043 | Hs. 73133 | metallothionein 3 (growth inhibitory fac | 5.19E−05 | 1.14 |
| 456523 | AI083668 | Hs. 50601 | hypothetical protein MGC10986 | 4.77E−04 | 1.14 |
| 414314 | BE312991 | Hs. 50535 | gb: 601150275F1 NIH_MGC_19 Homo sapiens c | 4.85E−03 | 1.14 |
| 433091 | Y12642 | Hs. 3185 | lymphocyte antigen 6 complex, locus D | 3.42E−03 | 1.14 |
| 402878 | | | ENSP00000217420*: BA122O1.1 (A novel prot | 2.18E−05 | 1.14 |
| 417721 | R11266 | Hs. 20755 | ESTs | 1.24E−03 | 1.14 |
| 416115 | BE147652 | | gb: RC1-HT0229-030500-111-a05 HT0229 Homo | 2.60E−05 | 1.14 |
| 401008 | | | Target Exon | 4.08E−04 | 1.14 |
| 442040 | AW294162 | Hs. 301062 | UDP-N-acetyl-alpha-D-galactosamine: polyp | 3.70E−03 | 1.14 |
| 437216 | H13364 | | gb: yl71b11.r1 Soares infant brain 1NIB H | 1.48E−03 | 1.14 |
| 401037 | | | C11000425: gi\|4507721\|ref\|NP_003310.1\| ti | 2.01E−10 | 1.14 |
| 441813 | BE564575 | Hs. 151093 | hypothetical protein DKFZp434G1415 | 5.42E−04 | 1.14 |
| 404187 | | | NM_019602: Homo sapiens butyrophilin-like | 1.28E−03 | 1.14 |
| 445071 | AI280246 | Hs. 149504 | ESTs | 2.53E−03 | 1.14 |
| 444903 | AW103062 | Hs. 160391 | ESTs | 6.10E−03 | 1.13 |
| 458863 | BE281219 | Hs. 179260 | chromosome 14 open reading frame 4 | 3.83E−03 | 1.13 |
| 438327 | H87407 | | chorionic gonadotropin, beta polypeptide | 2.14E−05 | 1.13 |
| 407404 | AF040257 | | gb: Homo sapiens TNF receptor homolog mRN | 5.77E−03 | 1.13 |
| 459014 | AW292935 | Hs. 100057 | Human DNA sequence from clone RP11-550O8 | 7.10E−03 | 1.13 |
| 414182 | AA136301 | | KIAA1105 protein | 7.83E−04 | 1.13 |
| 402869 | | | Target Exon | 6.45E−05 | 1.13 |
| 420772 | AW752656 | Hs. 222707 | KIAA1718 protein | 4.72E−04 | 1.13 |
| 404504 | | | Target Exon | 1.50E−03 | 1.13 |
| 456825 | H67220 | Hs. 169681 | death effector domain-containing | 4.95E−03 | 1.13 |
| 405593 | | | Target Exon | 4.73E−04 | 1.13 |
| 420167 | AA256113 | Hs. 60659 | ESTs, Weakly similar to T46471 hypotheti | 8.70E−04 | 1.13 |
| 454077 | AC005952 | Hs. 37062 | insulin-like 3 (Leydig cell) | 2.15E−03 | 1.13 |
| 412755 | BE144306 | Hs. 179891 | ESTs, Weakly similar to P4HA_HUMAN PROLY | 1.78E−03 | 1.13 |
| 417639 | R10467 | Hs. 339793 | ESTs, Weakly similar to ALU1_HUMAN ALU S | 6.58E−03 | 1.13 |
| 449684 | AI659166 | Hs. 207144 | ESTs | 8.20E−03 | 1.12 |
| 442770 | AI017267 | Hs. 203763 | ESTs | 6.56E−03 | 1.12 |
| 402255 | | | NM_031417*: Homo sapiens MAP/microtubule | 4.32E−04 | 1.12 |
| 448943 | AI608810 | Hs. 193288 | ESTs | 1.93E−04 | 1.12 |
| 438254 | AW269804 | Hs. 153019 | ESTs | 8.64E−03 | 1.12 |
| 428653 | AA135370 | Hs. 188536 | Homo sapiens cDNA: FLJ21635 fis, clone C | 1.94E−03 | 1.12 |
| 406146 | | | NM_003319*: Homo sapiens titin (TTN), mRN | 8.57E−03 | 1.12 |
| 446947 | AF146747 | Hs. 232165 | polycythemia rubra vera 1; cell surface | 5.74E−04 | 1.12 |
| 435264 | AI247780 | Hs. 117036 | ESTs | 2.54E−03 | 1.12 |
| 444459 | AI680624 | Hs. 148676 | ESTs | 8.40E−04 | 1.12 |
| 444279 | U62432 | Hs. 89605 | cholinergic receptor, nicotinic, alpha p | 4.90E−05 | 1.12 |
| 403722 | | | C4001282: gi\|7662006\|ref\|NP_055550.1\| KIA | 3.96E−03 | 1.12 |
| 452316 | AA298484 | Hs. 61265 | ESTs, Moderately similar to G786_HUMAN P | 2.13E−04 | 1.12 |
| 409382 | AA071244 | | gb: zm73g03.r1 Stratagene neuroepithelium | 4.57E−03 | 1.12 |
| 452947 | AW130413 | | gb: xf50f04.x1 NCI_CGAP_Gas4 Homo sapiens | 1.01E−04 | 1.12 |
| 446900 | AV660909 | Hs. 282705 | ESTs | 3.36E−03 | 1.12 |
| 447955 | BE544271 | Hs. 288390 | hypothetical protein FLJ22795 | 2.44E−07 | 1.12 |
| 451383 | AW239364 | Hs. 20242 | hypothetical protein FLJ12788 | 6.26E−03 | 1.12 |
| 408112 | AW451982 | Hs. 248613 | ESTs | 1.07E−03 | 1.12 |
| 404805 | | | ENSP00000252109*: KIAA1043 PROTEIN (FRAGM | 2.50E−04 | 1.12 |
| 403830 | | | NM_001328*: Homo sapiens C-terminal bindi | 1.78E−03 | 1.11 |
| 411507 | AW850140 | | gb: IL3-CT0219-261099-023-D11 CT0219 Homo | 6.34E−03 | 1.11 |
| 448169 | BE252749 | Hs. 20558 | hypothetical protein FLJ20345 | 4.28E−03 | 1.11 |
| 414125 | BE253197 | Hs. 334520 | hypothetical protein MGC15613 | 1.73E−03 | 1.11 |
| 442522 | AI087038 | Hs. 146592 | ESTs, Weakly similar to ALU7_HUMAN ALU S | 6.74E−04 | 1.11 |
| 447468 | AL109959 | Hs. 18632 | Homo sapiens mRNA full length insert cDN | 5.61E−05 | 1.11 |
| 418028 | R36212 | Hs. 235534 | ESTs, Weakly similar to ALU5_HUMAN ALU S | 8.67E−03 | 1.11 |
| 419274 | U09877 | Hs. 89864 | superkiller viralicidic activity 2 (S. c | 1.52E−03 | 1.11 |
| 450545 | AW135582 | Hs. 201767 | ESTs | 2.51E−06 | 1.11 |
| 457840 | AJ245874 | Hs. 4245 | chromosome 11 hypothetical protein ORF3 | 2.53E−03 | 1.11 |
| 404909 | | | NM_025213: Homo sapiens spectrin, beta, n | 5.66E−04 | 1.11 |
| 412419 | AW948630 | | gb: QV0-FT0001-050500-226-g05 FT0001 Homo | 4.87E−03 | 1.11 |
| 456126 | AA164743 | Hs. 187617 | hypothetical protein FLJ13941 | 6.48E−04 | 1.10 |
| 401450 | | | NM_031464*: Homo sapiens hypothetical pro | 5.69E−03 | 1.10 |
| 428680 | U69199 | | ESTs, Weakly similar to CGHU1S collagen | 7.32E−04 | 1.10 |
| 423499 | AW608884 | Hs. 28068 | ESTs | 5.44E−05 | 1.10 |
| 447755 | AI802667 | Hs. 161348 | ESTs | 6.52E−04 | 1.10 |
| 426070 | AF111170 | Hs. 306165 | Homo sapiens 14q32 Jagged2 gene, complet | 7.77E−05 | 1.10 |
| 407631 | AW574891 | Hs. 55067 | hypothetical protein MGC15437 | 2.49E−03 | 1.10 |
| 441548 | AW394255 | Hs. 127143 | ESTs | 2.03E−04 | 1.10 |
| 430820 | AF194815 | | immunoglobulin lambda variable 4-3 | 2.28E−03 | 1.10 |
| 456469 | NM_005109 | Hs. 95220 | oxidative-stress responsive 1 | 4.60E−05 | 1.10 |
| 420239 | NM_005544 | Hs. 96063 | insulin receptor substrate 1 | 8.63E−03 | 1.10 |
| 446541 | N73773 | Hs. 282950 | ESTs | 2.36E−03 | 1.10 |
| 417469 | AW189533 | Hs. 252748 | Homo sapiens cDNA FLJ20394 fis, clone KA | 8.97E−04 | 1.10 |
| 458126 | AW979136 | Hs. 124629 | ESTs | 1.82E−04 | 1.10 |
| 442364 | AA993149 | Hs. 129895 | ESTs, Moderately similar to TBX3_HUMAN T | 2.74E−04 | 1.10 |
| 420958 | AA309431 | Hs. 66 | interleukin 1 receptor-like 1 | 1.93E−04 | 1.10 |
| 431111 | AB033072 | Hs. 250015 | KIAA1246 protein | 3.00E−05 | 1.09 |
| 435773 | AW022047 | Hs. 41119 | otoraplin | 2.74E−04 | 1.09 |
| 431365 | AA504080 | Hs. 191958 | immunoglobulin superfamily receptor tran | 1.23E−03 | 1.09 |
| 415016 | N62934 | | ESTs | 2.17E−04 | 1.09 |
| 459233 | AI939966 | | gb: MR0-CT0015-160799-002-b06 CT0015 Homo | 9.36E−03 | 1.09 |
| 454063 | AF171928 | Hs. 37055 | fibroblast growth factor 5 | 5.43E−03 | 1.09 |
| 412518 | BE047637 | Hs. 334859 | hypothetical protein FLJ10297 | 3.60E−06 | 1.09 |
| 424324 | AA346316 | | gb: EST52440 Greater omentum tumor Homo s | 1.61E−03 | 1.09 |
| 412202 | AW900890 | Hs. 21782 | ESTs, Weakly similar to ALU7_HUMAN ALU S | 1.69E−03 | 1.09 |
| 453573 | AL042961 | Hs. 95111 | Homo sapiens cDNA: FLJ23080 fis, clone L | 2.27E−04 | 1.09 |
| 420599 | AW294971 | Hs. 172084 | Homo sapiens, clone IMAGE: 3627860, mRNA, | 4.38E−04 | 1.09 |
| 416416 | H73127 | Hs. 141269 | ESTs, Moderately similar to ZN91_HUMAN Z | 7.56E−03 | 1.09 |
| 405316 | | | C7002129*: gi\|3638957\|gb\|AAC36301.1\|(AC0 | 3.56E−05 | 1.09 |
| 404364 | | | Target Exon | 4.89E−03 | 1.09 |
| 445097 | W26975 | Hs. 46736 | hypothetical protein FLJ23476 | 3.64E−04 | 1.09 |
| 431807 | AW970894 | Hs. 324430 | ESTs, Moderately similar to 1207289A rev | 1.66E−04 | 1.09 |
| 439760 | AL355741 | Hs. 21641 | Homo sapiens mRNA full length insert cDN | 1.36E−04 | 1.09 |
| 425952 | BE336863 | Hs. 184456 | hypothetical protein | 6.92E−05 | 1.08 |
| 406138 | | | NM_000179*: Homo sapiens mutS (E. coli) h | 5.55E−03 | 1.08 |
| 452155 | AA812225 | Hs. 45719 | KIAA0823 protein | 4.47E−04 | 1.08 |
| 422388 | AA310006 | | gb: EST181073 Jurkat T-cells V Homo sapie | 6.14E−03 | 1.08 |
| 459285 | BE407166 | Hs. 334812 | hypothetical protein DKFZp586K0717 | 4.84E−04 | 1.08 |
| 424397 | AI950320 | | gb: wp08d10.x1 NCI_CGAP_Kid12 Homo sapien | 7.44E−03 | 1.08 |
| 451285 | AW137912 | Hs. 227583 | Homo sapiens chromosome X map Xp11.23 L- | 8.92E−05 | 1.08 |
| 441395 | AI681412 | Hs. 176655 | ESTs | 3.36E−04 | 1.08 |
| 452703 | AW299887 | Hs. 213402 | ESTs | 2.32E−03 | 1.08 |
| 402338 | | | Target Exon | 4.16E−05 | 1.08 |
| 420988 | AW006352 | Hs. 159643 | ESTs, Weakly similar to T32554 hypotheti | 3.36E−03 | 1.08 |
| 409510 | AW877400 | Hs. 191782 | ESTs, Weakly similar to ALU1_HUMAN ALU S | 6.11E−04 | 1.08 |
| 449236 | AJ403126 | Hs. 26373 | Homo sapiens cDNA: FLJ23449 fis. clone H | 2.62E−03 | 1.08 |
| 420243 | AA256874 | Hs. 187631 | ESTs, Weakly similar to Y076_HUMAN HYPOT | 6.50E−03 | 1.08 |
| 404244 | | | Target Exon | 3.53E−03 | 1.08 |
| 423546 | AW341131 | Hs. 146345 | ESTs | 1.20E−04 | 1.08 |
| 417967 | BE244373 | Hs. 1119 | nuclear receptor subfamily 4, group A, m | 9.37E−05 | 1.07 |
| 418784 | T41418 | | gb: ph1h3_19/1TV Outward Alu-primed hncDN | 1.50E−03 | 1.07 |
| 412682 | AW983772 | | gb: RC3-HN0002-060400-012-h09 HN0002 Homo | 2.24E−03 | 1.07 |
| 417408 | F17211 | Hs. 86092 | Homo sapiens skeletal myosin light chain | 2.00E−04 | 1.07 |
| 452104 | NM_000197 | Hs. 477 | hydroxysteroid (17-beta) dehydrogenase 3 | 9.02E−04 | 1.07 |
| 408710 | Y10256 | Hs. 47007 | mitogen-activated protein kinase kinase | 3.05E−05 | 1.07 |
| 402124 | | | NM_031891: Homo sapiens cadherin 20, type | 3.36E−03 | 1.07 |
| 400470 | | | Target Exon | 5.96E−03 | 1.07 |
| 420357 | U94333 | Hs. 97199 | complement component C1q receptor | 3.40E−03 | 1.07 |
| 424173 | AW138201 | Hs. 101064 | Homo sapiens cDNA FLJ12777 fis. clone NT | 1.18E−03 | 1.07 |
| 440517 | AW139632 | Hs. 132246 | ESTs | 1.93E−04 | 1.07 |
| 438357 | AI042101 | Hs. 294107 | ESTs | 8.89E−03 | 1.07 |
| 428813 | AA436063 | Hs. 25734 | ESTs | 5.80E−03 | 1.07 |
| 406279 | | | C18000033*: gi\|5901944\|ref\|NP_008977.1\|e | 9.43E−03 | 1.07 |
| 402635 | | | KIAA0408 gene product | 9.81E−04 | 1.06 |
| 417329 | AI190413 | Hs. 8373 | ESTs | 9.11E−10 | 1.06 |
| 456059 | BE543127 | Hs. 336948 | Homo sapiens, clone IMAGE: 3530891, mRNA, | 1.83E−04 | 1.06 |
| 401532 | | | C15001264*: gi\|12737280\|ref\|XP_006682.2\| | 5.46E−04 | 1.06 |
| 403358 | | | NM_006898*: Homo sapiens homeo box D3 (HO | 1.30E−04 | 1.06 |
| 441250 | AA926736 | Hs. 131338 | ESTs | 6.75E−03 | 1.06 |
| 413772 | BE163499 | | gb: QV3-HT0460-160200-092-d02 HT0460 Homo | 1.16E−03 | 1.06 |
| 458522 | AW452054 | Hs. 161139 | ESTs | 2.12E−04 | 1.06 |
| 406755 | N80129 | Hs. 199263 | metallothionein 1L | 2.56E−06 | 1.06 |
| 438540 | AA810021 | Hs. 136906 | ESTs | 8.27E−05 | 1.06 |
| 402517 | | | Target Exon | 8.49E−05 | 1.06 |
| 422867 | L32137 | Hs. 1584 | cartilage oligomeric matrix protein (pse | 8.26E−03 | 1.06 |
| 433299 | AA582324 | | ESTs | 1.19E−04 | 1.06 |
| 457165 | AL353972 | Hs. 202613 | ESTs | 5.47E−04 | 1.06 |
| 440371 | BE268550 | Hs. 80449 | Homo sapiens, clone IMAGE: 3535294, mRNA, | 4.10E−03 | 1.06 |
| 414205 | BE382438 | | Homo sapiens, clone IMAGE: 3690478, mRNA, | 1.89E−03 | 1.06 |
| 421453 | AA234652 | Hs. 104555 | neuropeptide FF-amide peptide precursor | 3.52E−04 | 1.06 |
| 415515 | F11327 | Hs. 167406 | gb: HSC2VD101 normalized infant brain cDN | 1.44E−07 | 1.06 |
| 441638 | AW293202 | Hs. 133451 | ESTs | 6.88E−03 | 1.06 |
| 400576 | | | Target Exon | 1.45E−04 | 1.06 |
| 422221 | AA306649 | Hs. 169370 | FYN oncogene related to SRC, FGR, YES | 8.73E−03 | 1.05 |
| 440187 | AI814224 | Hs. 148565 | ESTs | 8.70E−03 | 1.05 |
| 410137 | AI244459 | Hs. 110826 | trinucleotide repeat containing 9 | 5.49E−03 | 1.05 |
| 436537 | AI201305 | Hs. 335027 | ESTs | 5.64E−03 | 1.05 |
| 454397 | AW974648 | | gb: EST386752 MAGE resequences, MAGM Homo | 3.26E−03 | 1.05 |
| 432767 | NM_014114 | Hs. 278928 | PRO0097 protein | 7.72E−03 | 1.05 |
| 456013 | T92048 | Hs. 324531 | gb: yd54g12.s1 Soares fetal liver spleen | 1.08E−04 | 1.05 |
| 438233 | W52448 | Hs. 56147 | ESTs | 9.94E−03 | 1.05 |
| 442400 | AW381148 | Hs. 169407 | 2,3-bisphosphoglycerate mutase | 6.12E−03 | 1.05 |
| 424648 | AA344576 | Hs. 17253 | gb: EST50478 Gall bladder I Homo sapiens | 2.96E−03 | 1.05 |
| 404907 | | | NM_025213: Homo sapiens spectrin, beta, n | 9.51E−06 | 1.05 |
| 409487 | H19886 | | gb: yn57a05.r1 Soares adult brain N2b5HB5 | 1.22E−04 | 1.05 |
| 422311 | AF073515 | Hs. 114948 | cytokine receptor-like factor 1 | 4.30E−04 | 1.05 |
| 431151 | BE207083 | | gb: ba10d10.y1 NIH_MGC_7 Homo sapiens cDN | 4.15E−03 | 1.05 |
| 458891 | AI659166 | Hs. 207144 | ESTs | 1.35E−05 | 1.05 |
| 452972 | M31732 | Hs. 31210 | B-cell CLL/lymphoma 3 | 5.22E−05 | 1.05 |
| 456767 | AI086412 | Hs. 129064 | Homo sapiens chromosome 19, cosmid F2216 | 1.04E−04 | 1.05 |
| 419017 | T83961 | | gb: yd76c08.s1 Soares fetal liver spleen | 4.83E−05 | 1.05 |
| 426738 | AA421097 | Hs. 291902 | ESTs | 7.01E−06 | 1.04 |
| 414386 | X00442 | Hs. 75990 | haptoglobin | 1.19E−06 | 1.04 |
| 436234 | AI809027 | | ESTs | 1.15E−04 | 1.04 |
| 437343 | AI521190 | Hs. 165983 | hypothetical C2H2 zinc finger protein FL | 1.58E−03 | 1.04 |
| 413042 | BE061618 | | gb: RC6-BT0252-151099-011-c05 BT0252 Homo | 4.40E−03 | 1.04 |
| 400765 | | | Target Exon | 2.94E−05 | 1.04 |
| 453989 | M63962 | Hs. 36992 | ATPase, H? exchanging, alpha polypeptide | 1.57E−03 | 1.04 |
| 422743 | BE304678 | Hs. 119598 | ribosomal protein L3 | 5.39E−04 | 1.04 |
| 454065 | BE394588 | | gb: 601311808F1 NIH_MGC_44 Homo sapiens c | 5.26E−05 | 1.04 |
| 405201 | | | Target Exon | 6.03E−03 | 1.04 |
| 436221 | AK001781 | Hs. 296543 | Homo sapiens cDNA FLJ10919 fis, clone OV | 4.68E−03 | 1.04 |
| 414376 | BE393856 | Hs. 66915 | ESTs, Weakly similar to 16.7 Kd protein [ | 1.13E−03 | 1.04 |
| 428633 | AW450428 | Hs. 112050 | ESTs | 1.15E−05 | 1.04 |
| 433821 | AW182416 | | ESTs | 1.96E−04 | 1.04 |
| 448401 | AI498509 | | ESTs | 3.15E−03 | 1.04 |
| 408278 | AW876813 | | phosphoglycerate dehydrogenase | 4.02E−05 | 1.04 |
| 405282 | | | Target Exon | 2.01E−03 | 1.04 |
| 439548 | AA086045 | Hs. 137313 | ESTs | 6.15E−04 | 1.03 |
| 444788 | AI871122 | Hs. 202821 | ESTs | 3.94E−04 | 1.03 |
| 430842 | AC002511 | Hs. 272485 | G protein-coupled receptor 41 | 8.52E−03 | 1.03 |
| 434179 | AI743448 | Hs. 116177 | ESTs | 3.72E−03 | 1.03 |
| 427827 | AA416577 | Hs. 189105 | ESTs, Weakly similar to NBR13 [H. sapiens | 8.89E−03 | 1.03 |
| 412139 | BE044976 | | gb: hn25b10.x1 NCI_CGAP_Thy7 Homo sapiens | 5.90E−03 | 1.03 |
| 445500 | AW451938 | Hs. 257512 | ESTs | 4.34E−03 | 1.03 |
| 452151 | AI190405 | Hs. 143127 | ESTs | 8.32E−03 | 1.03 |
| 412474 | AI791451 | | gb: ni50c09.y5 NCI_CGAP_Ov2 Homo sapiens | 1.42E−03 | 1.03 |
| 407382 | AA503620 | | gb: ne49b08.s1 NCI_CGAP_Co3 Homo sapiens | 5.88E−06 | 1.03 |
| 413805 | AW945740 | Hs. 289293 | ESTs | 1.11E−03 | 1.03 |
| 448020 | H06022 | Hs. 334916 | Homo sapiens, clone MGC: 17331, mRNA, com | 9.93E−03 | 1.03 |
| 407586 | Z37544 | Hs. 37121 | phospholipase C, beta 3 (phosphatidylino | 1.14E−04 | 1.03 |
| 438998 | AF075064 | | gb: Homo sapiens full length insert cDNA | 2.30E−03 | 1.03 |
| 402022 | | | Target Exon | 8.26E−04 | 1.03 |
| 442321 | AF207664 | Hs. 8230 | a disintegrin-like and metalloprotease ( | 1.86E−06 | 1.03 |
| 459375 | BE251770 | | gb: 601112470F1 NIH_MGC_16 Homo sapiens c | 3.01E−04 | 1.03 |
| 418003 | AA976107 | Hs. 128691 | Homo sapiens mRNA for KIAA1762 protein, | 1.54E−03 | 1.03 |
| 439694 | AA843915 | Hs. 54707 | ESTs | 3.55E−04 | 1.02 |
| 458318 | BE177830 | | gb: RC1-HT0598-120400-022-b08 HT0598 Homo | 7.76E−04 | 1.02 |
| 435680 | H50946 | Hs. 284183 | Homo sapiens galectin-related inhibitor | 1.38E−05 | 1.02 |
| 445179 | AI949743 | Hs. 224768 | ESTs | 4.44E−03 | 1.02 |
| 455932 | BE158451 | | gb: CM1-HT0390-301199-052-b01 HT0390 Homo | 2.79E−04 | 1.02 |
| 420429 | AW293291 | Hs. 182382 | ESTs | 7.07E−03 | 1.02 |
| 446185 | AI279191 | Hs. 149454 | ESTs, Weakly similar to DSR6_HUMAN DOWN | 3.70E−03 | 1.02 |
| 410628 | AI131408 | Hs. 68756 | ESTs, Moderately similar to similar to s | 1.52E−05 | 1.02 |
| 428739 | AA700158 | Hs. 301755 | Homo sapiens cDNA FLJ11465 fis, clone HE | 9.54E−03 | 1.02 |
| 409651 | H96643 | Hs. 283565 | FOS-like antigen-1 | 4.45E−09 | 1.02 |
| 445311 | AW027556 | Hs. 345522 | ESTs | 9.11E−03 | 1.02 |
| 403579 | | | Target Exon | 4.78E−04 | 1.02 |
| 403957 | | | Target Exon | 1.30E−04 | 1.02 |
| 415471 | F09747 | Hs. 268707 | ESTs | 6.85E−04 | 1.02 |
| 440763 | AB028988 | Hs. 7407 | KIAA1065 protein | 8.27E−04 | 1.01 |
| 445474 | AI240014 | Hs. 259558 | ESTs | 5.32E−04 | 1.01 |
| 408690 | AW864542 | | gb: PM4-SN0016-120500-003-h02 SN0016 Homo | 1.89E−03 | 1.01 |
| 442574 | AI932361 | Hs. 238039 | hypothetical protein FLJ11090 | 5.28E−04 | 1.01 |
| 448778 | AF074913 | | gb: Homo sapiens transcription factor Pax | 8.48E−03 | 1.01 |
| 400425 | AY004252 | Hs. 287385 | PR domain containing 12 | 3.58E−06 | 1.01 |
| 438326 | AW575901 | Hs. 123230 | ESTs, Weakly similar to Z195_HUMAN ZINC | 9.93E−04 | 1.01 |
| 439698 | AW779654 | Hs. 55876 | ESTs | 3.98E−03 | 1.01 |
| 419189 | T95862 | Hs. 112318 | 6.2 kd protein | 5.70E−03 | 1.01 |
| 436407 | T88803 | Hs. 237517 | ESTs, Weakly similar to TIM_HUMAN PROBAB | 3.93E−04 | 1.01 |
| 414630 | BE410857 | Hs. 16064 | gb: 601301177F1 NIH_MGC_21 Homo sapiens c | 9.04E−03 | 1.01 |
| 409294 | BE546175 | Hs. 71746 | hypothetical protein FLJ11583 | 2.83E−03 | 1.01 |
| 450184 | W31096 | Hs. 237617 | Homo sapiens, clone IMAGE: 3447394, mRNA, | 8.64E−04 | 1.01 |
| 457771 | AI814635 | Hs. 187117 | ESTs | 3.92E−03 | 1.01 |
| 451595 | AW965569 | Hs. 20996 | ESTs | 4.46E−04 | 1.01 |
| 416559 | AI039195 | Hs. 128060 | ESTs | 6.29E−03 | 1.01 |
| 453680 | AL079647 | Hs. 14485 | Homo sapiens cDNA: FLJ23220 fis, clone A | 5.45E−03 | 1.01 |
| 424365 | AI653164 | Hs. 128665 | ESTs, Moderately similar to I54374 gene | 4.16E−03 | 1.01 |
| 419357 | AW955121 | Hs. 55148 | hypothetical protein FLJ14466 | 6.12E−04 | 1.00 |
| 450991 | AI751020 | Hs. 313648 | gb: cnO7g05.x1 Normal Human Trabecular Bo | 5.11E−05 | 1.00 |
| 419861 | AW081795 | Hs. 233465 | ESTs | 4.72E−03 | 1.00 |

Pkey: Unique Eos probeset identifier number
ExAccn: Exemplar Accession number, Genbank accession number
UnigeneID: Unigene number
Unigene Title: Unigene gene title
R1: T-test p-value
R2: Difference of the average standardized value of Hep C− Liver vs. the average standardized value of Hep C+ Liver

TABLE 12B

| Pkey | CAT Number | Accession |
|---|---|---|
| 454947 | 1083824_1 | AW846590 AW846615 AW846584 AW846592 AW846621 AW846610 |
| 455765 | 6676_13 | BG620787 AV761949 BG980132 AV761951 BE082222 BG979177 BF329571 BF908591 BE082279 BE081722 BG978604 |
| 455919 | 1527074_1 | BE157195 BE157345 BE157130 BE157368 BE157136 BE157134 BE157202 BE157305 BE157109 BE157294 |
| | | BE156971 BE156997 BE157374 BE156970 BE157366 BE157356 BE156973 BE157358 BE157300 |
| 412998 | 1343218_1 | BE046254 BE046673 BE046253 |
| 417574 | 2134673_1 | R00348 R09593 |
| 456636 | 433615_1 | AW954166 AV723710 AW014769 AA916838 AW205448 AA476474 AI214918 BE550871 AI299143 AI685787 |
| | | AI810480 AA476475 AI681494 AI378542 BF222633 AA769645 AA299751 AA299752 |
| 409840 | 915929_1 | AW502122 AW501663 AW501720 AW502125 |
| 432745 | 112643_1 | AA658826 AI821926 AI791191 AA635129 AA564492 |
| 454810 | 1073338_1 | AW832915 AW832906 AW832788 AW832917 AW832776 AW832913 |
| 411772 | 300555_1 | BE154090 AW861539 BF921665 BF350632 BF350631 AW904851 BE170301 BE154336 BE154275 |
| 407457 | 24680_1 | AB044546 AJ242724 BF760529 BF946992 BG983676 |
| 450703 | 35938_2 | AK056299 AI669104 AI378016 AI420515 AW297177 AI702739 AA889785 AA011202 |
| 436871 | 57685_1 | AA070431 AA121992 AA076561 AA080879 |
| 459235 | 26808_3 | AW814516 AW815927 AW814504 BF375203 AW814522 AW814521 AW814524 BF375206 AW814518 AW814517 |
| | | AI940652 BF837881 AW751232 BF374342 AW176453 AW814505 BF886651 AW814525 BG001382 BE933380 |
| 426505 | 1139_4 | BG177042 BE185582 BE185655 BE185657 AA380194 |
| 423053 | 367623_1 | BE314223 AA320990 BE312679 BI054000 BI324838 AW057717 |
| 444163 | 682245_1 | BG403189 AI148521 AI184746 AI126098 R05933 BI057330 |
| 440730 | 1648529_1 | BE561722 BE268773 BE268956 |
| 447721 | 1889_2 | BC013767 AL572931 BE742185 BI520113 AW959076 AI341487 AI623222 BM091074 AW593800 AI983635 AW275114 |
| | | AI952164 BM091378 AA977038 AW513859 AI801910 AW273202 AW166266 AW337946 AI086791 BE907359 |
| | | AW273147 AI453134 AA250733 AW072844 AI818468 AI561259 AW470887 AW300481 AW103087 AW175624 |
| | | BE048584 BF063936 AI207341 AW193240 AW193322 AW264492 |
| | | AI682412 AI631778 AI669677 AI128695 AA448630 AA456607 BF313680 BG898294 BI195544 BG421755 |
| | | BI760100 BE383304 BF329916 BF526599 |
| | | AA385255 BI520887 AA410939 AA448721 BF525380 BG423666 BI761786 BF944570 |
| 455973 | 1561647_1 | BF352282 BE175424 BE175418 BE175383 |
| 454707 | 1067650_1 | AW814808 BG999545 AW814989 AW814852 |
| 452621 | 2071061_1 | AI910230 AI910262 AL134108 N44030 |
| 419555 | 252042_1 | AA244401 AA244416 |
| 451402 | 1958047_1 | AI793167 H39479 |
| 434646 | 15463_1 | AF147445 AA130804 AA069114 AA069113 |
| 409795 | MH905_27 | AI498073 AI934808 AW374654 BI036066 BE709215 AA077985 BI831355 |
| | | BF932450 BF770705 BF379220 AA077682 |
| | | AA076814 AA076875 BI027970 AA077787 AA077311 BF811798 AW393752 BF327269 AA077305 AA078114 |
| | | BF800885 BF942930 AW903223 BF916052 |
| | | AW903170 AW903224 BF352664 BF379209 |
| 442793 | 417820_2 | BG741247 BG741022 AI017798 AI953594 AW445065 AI245087 |
| 413537 | 1375441_1 | BE146866 BE146865 BE146867 |
| 455968 | 1557068_1 | BE168828 BE168823 BE168830 BE168820 BE168931 BE168826 BE168928 |
| 416648 | 1982787_1 | R97488 H71377 R97368 |
| 458867 | 1246993_1 | AW995393 AJ403118 |
| 425526 | 1076747_1 | AW962995 AW955306 AA359933 AA358889 AW837746 BG961280 |
| | | AW837697 AW837755 |
| 408099 | 7104_17 | AW151646 AW151645 AW151637 BI562740 |
| 434884 | 10696_1 | AF161340 AF150140 AV738842 |
| 438687 | 2575583_1 | AI674070 AI239526 AI240515 AA814084 |
| 441184 | 1816784_1 | BE715995 BE715973 AI204080 AA954474 AA922009 |
| 410312 | 1001291_1 | AW851102 AW851181 AW850953 AW851110 AW629671 |
| 423103 | 879332_1 | BE315237 AA322029 |
| 447135 | 579607_1 | T58148 AW516579 AW059603 |
| 426254 | 7308_1 | BC017081 AL539521 AL524509 AL556058 AL541690 |
| | | AL543200 BF974751 BE514793 AW247083 BE183238 AA376427 |
| | | AF202637 NM_022156 BI554151 AL559157 BE909859 AW009244 |
| | | AW043786 AW273850 AI871111 AI889050 AL045428 AI884485 AI554001 |
| | | AI375841 AI469097 BF741351 AW188439 AA410448 AW515248 BE301590 |
| | | AW007777 AI148210 AI805423 BE784190 AV658045 AI093347 AI308976 |
| | | AI249682 AI206484 AI493895 AA973765 F29582 AA335219 AA379270 F37119 |
| | | AA026496 BE645159 AA617996 AI886986 BM144558 BC003659 |
| | | BG739795 BE729530 BG334810 BE746857 BE390302 BM354208 BF757460 |
| | | AA284639 AW967315 BE769162 W96068 BM477081 BG490980 BM013665 |
| | | BE735761 BE257483 BE257427 BG255544 AI474585 AL569826 BG337562 BG491737 |
| | | AL559158 BG822257 AL554642 AL570587 AA588594 AL041071 |
| | | AA554020 AA287274 AA810799 AA729332 AA659019 BE439973 AA335218 AA639556 |
| | | AA610680 R16774 H26778 AA026419 AA010840 BF830072 |
| | | R50167 AL577377 AA622659 F25241 BF880480 AA872445 BE386621 AL571579 |
| 415160 | 1861867_1 | T82802 R08505 D78670 |
| 416397 | 1969080_1 | T91431 H80874 H53035 |
| 412371 | 1172992_1 | AW946709 AW946621 AW946699 AW946695 AW946691 |
| | | AW946697 AW946666 |
| 416323 | 1964997_1 | H47929 N78108 N72630 |
| 412433 | 413465_1 | AV698615 AA102400 AA327633 AA102401 AI970895 |
| 455663 | 1490406_1 | BE065460 BE065619 BE065633 BE065626 BE065679 BE065617 |
| 433010 | 1234715_1 | AW970018 BE843649 BE843644 AA573622 R08736 AA573669 |
| 422800 | 11320_9 | BE545343 AW997954 |
| 414648 | 316607_1 | BF126232 H54463 BF950132 H54381 BF944624 BE393262 |
| 437093 | 325853_1 | BF955573 AA744334 AA745434 AA744302 |
| 445481 | 504944_1 | AW003346 AI808038 AI671563 AI240190 AW661846 AI286080 |
| 410922 | 1064329_1 | AW810845 AW810831 AW810661 AW810816 AW810772 AW810666 |
| | | AW810865 AW810859 AW810670 AW810805 AW810856 |
| | | AW810819 AW810852 AW810722 AW810699 |
| 412044 | 1149589_1 | BE086167 AW887835 AW887682 AW887677 BE178621 |
| | | AW887676 AW887674 |
| 448464 | 661708_1 | AW196419 AI522053 AI650395 |
| 432909 | 137712_1 | AA702596 AA570111 AI348435 AW192161 |
| 415597 | 1876403_1 | T74345 F12588 Z44384 |
| 440075 | 1648556_1 | BE559806 R12767 BE268806 |
| 441194 | 138512_1 | BE275382 AA703515 BE166690 BG334067 |
| 432092 | 34124_1 | AA583908 |
| 454537 | 15351_6 | BG421219 BI488939 BE391114 BE391493 BG764533 |
| | | BE276157 BE388289 |
| 446403 | 488811_1 | BE549932 BE467906 BE326357 BG149226 AI298565 |
| | | AW452904 AW449414 F04362 |
| 441662 | 2674073_1 | AI807406 AI798919 AA948548 |
| 411154 | 1071475_1 | AW819848 AW819850 AW819863 |
| 448544 | 1139239_1 | BM476470 AW902443 AI535819 |
| 430725 | 1597397_1 | AI478920 AI478907 AA485056 |
| 422579 | 15032_4 | BG035571 BM457635 AA312626 |
| 432081 | 1237080_1 | AA525888 AA525391 AA984238 AW972788 BE087083 |
| 453196 | 505145_1 | AW003567 AI963955 AI990231 |
| 421798 | 3042_4 | BC017829 AW276646 AI984209 AA663933 AA634104 AA551528 |
| | | AA634041 AA298038 BG483990 T89297 BF853958 H64685 |
| | | T90329 T60644 T57747 BF852694 T92529 BG482852 BF883064 BF883066 N74880 |
| | | AA829796 N90716 |
| 454803 | 1072913_1 | BI468492 AW862380 AW860148 AW821887 AW821863 |
| | | AW821894 AW821870 AW862378 AW862351 |
| 413619 | 1524553_1 | BE153524 BE153576 BE153583 |
| 428177 | 8737_7 | AA779866 AA423967 AA423968 |
| 411288 | 1074988_1 | AW835517 AW835513 AW835511 |
| 418551 | 2261897_1 | AL564778 R99167 T99393 T75517 |
| 415449 | 1873127_1 | H15034 T17195 F09069 |
| 457677 | 1205688_1 | AI374612 AA628890 |
| 454908 | 1078884_1 | AW840458 AW840400 AW840401 AW840535 AW840539 |
| | | AW840532 AW840538 |
| 413737 | 1551611_1 | BE160704 BE160849 BE160896 BF351011 |
| 454186 | 645362_1 | BE141775 BE141030 BE141467 BE141520 BE141028 BE141492 |
| | | BE141477 BE141460 AW177599 BE141456 BE141753 |
| | | AW177597 AW177582 AW807582 AW177585 BE141761 AW177598 AW177579 AW177583 |
| | | AW177587 BE141749 BE141489 BE141474 BE141512 BE141751 |
| | | BE141024 AW177581 BE141750 |
| 413961 | 566812_1 | BG251042 BE266372 BE207098 |
| 454598 | 1063474_1 | BF374577 AW809840 AW809996 AW809798 AW809695 AW809646 |
| | | AW809738 BF374582 AW809716 AW809826 AW809802 |
| | | AW809747 AW810152 |
| 417313 | 1943_33 | W01148 AA195602 N40632 |
| 410285 | 2930719_1 | AA083790 AA083609 AA112048 |
| 408432 | 288076_1 | AW811262 BF905430 BF908283 BF368183 BF907357 BF908452 |
| | | BF908282 BG001786 BF908281 BF907042 BF908278 BF908277 |
| | | BF908451 BF368225 BF379361 BF907348 BF908280 AW195262 R27868 |
| 449405 | 78886_1 | AA203114 R00660 H83070 AA001350 H70366 R95157 |
| 431357 | 1235874_1 | AW971393 AA503896 T81154 AA507148 |
| 428568 | 738084_1 | BE259137 BE251523 |
| 415037 | 980243_1 | AA159478 AW901089 AW593155 AA160437 |
| 440190 | 1030220_1 | AW848781 AW848490 AW849062 AW752597 AW752699 |
| 458146 | 114488_2 | AW589833 AI652176 AI216328 AI286094 |
| 409975 | 926611_1 | AW839324 AW516213 BE142635 |
| 419299 | 36328_1 | BM477587 BE675426 AA827059 AI597639 AI571409 AI719948 |
| | | AI311085 AA953361 AI498787 AI364049 AI311084 BF871020 |
| | | BF549868 AI356384 AA236660 |
| 400111 | 24179_2 | NM_006156 D23662 BF038671 BI670321 BI603145 BI666956 BG716628 BG707476 |
| | | BG768814 BE312007 BF125930 |
| | | BF037916 BG942671 AI752472 AW009362 BE379126 BI198555 BF126026 N28289 BE388301 BG327102 AI571450 |
| | | BF038400 AI884649 AI718962 AI742314 |
| | | AA977058 AI150699 BG944784 AA187402 BG283893 AV712007 W01301 BG774109 W77840 C15672 BI670016 |
| | | AI752473 AA484409 C14921 N41745 C15220 |
| | | F30164 AA133181 BF768974 H30334 AA034968 AA384232 AA353297 AW407023 AA337516 F36177 AA374444 |
| | | AA402758 AI141545 BI021470 AA973914 |
| 436375 | 46760_1 | AJ227870 AW798823 AW798704 |
| 409818 | 914614_1 | AW502932 AW500969 AW501134 AW501533 |
| 453741 | 1031156_1 | AL120304 AW855522 AW753631 |
| 439665 | 24497_1 | AF086498 AW955697 W93979 AI936062 AW268568 AA364461 W93980 BI914847 BI523483 |
| 455977 | 1562520_1 | BE176686 BE176477 BE176484 BE176679 |
| 457424 | 117833_1 | BM041161 BM040921 BM040887 AW876082 AA513157 BM041809 BM041314 AA747408 |
| | | AA747397 AA736702 AA745146 AA648638 |
| 430204 | MH1094_26 | AI821606 AI826629 AI304327 AI660560 AW050605 AI732165 AW009962 AI262416 |
| | | AI695625 AA469031 AI791844 AA573825 AA618335 |
| 455638 | 1489140_1 | BE142908 BE063665 |
| 450799 | 819959_1 | W31274 AW407504 AI738877 |
| 444207 | 9172_3 | BE739425 AA514221 AA865491 AI828293 AA470456 AI276739 AA169357 BE932464 AA514889 |
| | | AW819039 AW819083 BE843048 AI432496 |
| | | AI470335 AI247243 BG533994 AA513783 AI887309 AA528036 AW972006 AW873028 AI924914 AI818810 |
| | | AW152378 AW084946 AI521413 |
| | | AI669583 BE932521 AI581370 BE180238 AW089750 AW771461 AW089714 AI590949 AI819148 AA731056 |
| | | BF815234 BF911506 AA235803 |
| | | AA485373 AI735658 AW393133 AW073080 AI707637 BF353320 BE843111 AW819036 AW393135 BG697291 |
| | | AV648670 AV654332 AV687530 |
| | | BG566964 AI807430 AI676072 AA837010 AI452482 AI625817 AW241750 BE048616 AI290928 AI680714 AA485530 |
| | | BE175687 AV648513 |
| | | AW130312 AI000556 AA632893 BE674169 BF001208 AA948166 BE175650 AA524664 AA490345 AI244948 |
| | | AA602956 AA483492 AA918178 |
| | | AW802049 BG675859 AV658871 BG678060 AI565004 AW819026 BE843092 AV686437 AV723049 BG616948 |
| | | AI911647 AI743490 AI091096 |
| | | BE857251 AI962074 AA049027 AW769317 AA343477 AA640112 BF876213 R82948 H26425 H82876 BE843095 |
| | | BE843140 BG536641 BG617830 |
| | | AA235802 BE774985 BE006682 BF342375 AA903144 BF338083 BF984258 AV657996 AI749532 BE768614 |
| | | BE857252 BE932516 BE768573 |
| | | AV657993 AV657777 AV752631 BE774974 T55847 BF095761 BF911511 BE710793 BE180119 BG617338 |
| | | H45942 T55897 AV657718 BG563497 |
| 455194 | 1107901_1 | AW947513 AW864536 AW864318 |
| 413309 | 455217_1 | BF315003 BE312608 R10565 |
| 416115 | 863707_2 | BE147652 BG990288 |
| 437216 | 645_11 | R61679 AA746905 H13364 BF185622 N29108 |
| 438327 | 18001_1 | NM_033183 NM_033043 NM_000737 BC006290 J00117 |
| | | BG479175 AU138071 AI188721 AI188454 AI188947 |
| | | AI188781 AI188729 AI200658 AI148857 AI188639 AI209155 AI189616 AI189595 AI189275 AI192447 AI188604 |
| | | AI200457 AI188391 AI184224 AI190013 AI189641 |
| | | AI207360 AI148695 AI148590 AI149163 BF589174 AI188348 AI189842 AI188498 AI148904 AI138197 AI417614 |
| | | AI126101 AI160579 AI160936 AI160814 |
| | | AI123896 AI189442 AI149591 AI131286 AI138448 AI149559 AI128296 AI168482 AA330755 NM_033142 |
| | | AI192459 AI741512 AI206390 AI186215 |
| | | AI148647 AI189807 AI183280 AI200559 AI188964 AI188303 AI200022 AI186353 AI219044 AI200270 AI189555 |
| | | BG200016 BG210411 AI186465 |
| | | AI186218 AI189968 AI149039 AI273580 AI291937 AI276216 AI188689 AI157454 AI200375 AI184348 AI221605 |
| | | AI871881 BF432300 AI128424 |
| | | AI868741 AI219133 H93668 AW451899 H95381 C18834 AA331267 AA330978 AL548158 AI149571 AI149072 |
| | | AU155932 R68113 R26086 AA330414 |
| | | AL551303 AI188830 AI188824 AI206355 AI453626 AI189364 AI160692 AI189068 AI219266 T49171 AA330939 |
| | | AI130720 AI151301 AI199943 |
| | | AI221019 AI186330 AI149467 AI198460 AI220923 AI161082 AI654493 AI184784 AI219589 AA232905 AI160705 |
| | | AI189453 T47334 N30758 K03181 |
| | | AL551349 BG434672 N40745 N41758 T83001 R71610 AA331230 N39958 AA330844 BE901661 AU135260 |
| | | H86992 H87407 AA330968 AA330717 |
| | | AA330761 AU139360 N42631 AA333923 AA330974 BG435249 AA331021 AA331049 N41851 AA330831 R23289 |
| | | H87658 AA331043 AA330803 |
| | | AL548120 BG479085 AA330609 AA331023 AA331039 BE733206 BE732062 W76381 R63351 BF060995 N56715 |
| | | N56709 N40038 T52077 N41710 |
| | | H94535 BI088367 N41834 H94543 AA330643 N47829 R23494 BG434117 N42013 R68922 AA331012 N40479 |
| | | D78974 R67480 H56737 N40793 |
| | | N41357 N41591 N41596 H93655 R68162 H87630 T85052 R01541 AI219422 AI221014 AI219055 AI221580 AI220780 |
| | | T28278 AI219166 AI220865 |
| | | N27381 N27073 N41361 N42190 N57328 AI149426 N57248 H86986 W71989 N30684 N30822 T85053 AI188728 |
| | | AI168533 H95374 H94451 T49170 |
| | | BF369834 N29831 N31955 N30313 N32233 AI221552 AI126906 AA400910 T90191 R23214 AI138431 AI128407 |
| | | AI188492 AI186418 BF109879 |
| | | N26397 AI188872 AI149447 N30216 AI431595 AI219009 N30817 N30833 AI148774 AI188715 AI149156 BG774563 |
| | | H95176 H94991 H94347 |
| | | N32759 N32604 H12587 H93656 H94015 N29701 N30276 N27302 BF999045 N29730 R68815 H87631 H86993 |
| | | R63352 H95135 H94446 H87464 |
| | | H95207 N31616 NM_000894 AV751658 AV752236 AI051683 AV748886 T29543 AA371971 AA371978 |
| | | AA371346 AV753384 AA372026 AA371909 |
| | | AV745740 AV746068 AV751144 AV747923 AV745447 AV748231 AV745712 AA063496 N56646 H87574 AW951441 |
| 414182 | 2917943_1 | AA136321 AA136301 AI381776 |
| 409382 | 110743_1 | AA071477 AA071244 |
| 452947 | 625171_1 | AI932362 AW130413 |
| 411507 | 1088866_1 | AW850140 AW850195 AW850192 |
| 412419 | 1175957_1 | AW948630 AW948616 AW948604 AW948623 AW948627 AW948611 AW948605 |
| | | AW948610 AW948626 AW948634 |
| | | AW948631 AW948615 AW948607 AW948602 AW948633 AW948628 |
| 428680 | 41659_1 | AB075857 U69199 AL554057 BE550443 AI125387 AI125388 AI372992 AI686618 AA583075 |
| | | AW079720 AI948894 AI342763 |
| | | AI982591 AW779115 AI560632 BF001153 AI623930 AA447536 BG911172 AA480680 BI197703 BE294318 AL530842 |
| | | AL562327 AA448518 |
| | | AA432018 BE264771 BE259722 BF930927 |
| 430820 | 32425_1 | AF194815 AF194816 |
| 415016 | 1806690_1 | N62934 BE858324 N46417 N46363 N46427 N46358 D19793 H99021 |
| 459233 | 3170768_1 | AI939966 AI939976 AI939951 AI939981 AI939959 AI939988 |
| 424324 | 808543_1 | AW954536 AA338802 BE160193 AA346316 |
| 422388 | 870372_−1 | BE061034 |
| 424397 | 893246_1 | AI950320 AA340023 |
| 418784 | 2306231_1 | T41418 T41379 T41320 |
| 412682 | 1242945_1 | AW983772 AW983837 AW983730 AW983769 AW983836 AW983835 |
| 413772 | 1553630_1 | BE163499 BE163572 BE163580 BE163565 BE163503 BE163510 |
| 433299 | 1752_1 | AK056027 AL120636 AA582324 AA582305 AI459497 BI047007 AL589306 AI688987 |
| | | AI922469 AA350721 AI085972 |
| | | AF131844 AW954598 AW957578 AA350710 AI884831 R37119 AA666294 AA350709 BF724097 AA203665 |
| | | AI089250 AA708285 BF831652 |
| | | AW104744 AW104745 AW129091 AV724110 AW805934 AV682207 AA563965 W93840 AW104743 AW009432 |
| | | AI085993 BF839379 T79879 |
| | | BC019656 AA975816 AI953836 BE350274 AW840298 BI043880 BG913327 AW839960 T07306 R53951 |
| | | AI299256 AI961918 AI190874 AA724491 |
| | | F04512 BG997879 R08567 BE810546 |
| 414205 | 1266364_1 | BF306238 BE295439 BE382708 BE383262 BF315437 BI199567 BE382438 |
| 454397 | 118552_1 | AW974648 AA649671 AA652153 BF875948 AA078582 |
| 409487 | 815197_1 | H19886 T10231 AW402806 |
| 431151 | 1570517_2 | BE207083 |
| 419017 | 2348209_1 | T83961 T79425 T85415 T79511 |
| 436234 | 141336_1 | AW269942 AI809027 AI799097 AA707010 |
| 413042 | 1487648_1 | BE061618 BE061640 BE061619 |
| 454065 | 517162_1 | BE394588 AW024754 BE183167 BE183166 BE378353 |
| 433821 | 72553_2 | AK054648 AW182416 AA609856 AA918195 AA634398 AA927922 AA868718 |
| | | AI290470 AI338870 Z25302 T30841 |
| | | T30825 F29302 AA436128 BF095986 BI829584 AA853991 AA778707 |
| 448401 | 507788_1 | AW007014 AI498509 AW156947 AI933042 AW370727 AW370779 AW370731 |
| | | AW370726 AW370764 |
| 408278 | MH337_68 | BG422281 BG422108 AW177973 BG750536 AW876813 AW876814 |
| 412139 | 1154826_1 | AW895327 AW895462 BE044976 |
| 412474 | 1022404_1 | BE296601 BG829182 AI791451 BE019234 AI791288 AA111939 BG954837 |
| 438998 | 52228_1 | AF075064 R89255 R89256 |
| 459375 | 619648_−3 | BE251770 |
| 458318 | 1561764_1 | BE177830 AF114068 BE175531 BE175603 BE175601 BE177658 |
| 455932 | 1528183_1 | BE158451 BE158453 BE158585 BE158584 BE158454 BE158465 BE158464 |
| 408690 | 105725_1 | BG563152 BF846777 BF849354 BF849359 BF846636 BF849201 BF849356 |
| | | C16931 AA056717 AW864542 AW882724 |
| | | AA056567 |
| 448778 | 7800_1 | AW505435 U62539 |

Pkey: Unique Eos probeset identifier number
CAT number: Gene cluster number
Accession: Genbank accession numbers

TABLE 12C

| Pkey | Ref | Strand | Nt_position |
|---|---|---|---|
| 404473 | 8079921 | Plus | 22639-22773 |
| 402845 | 9369286 | Plus | 160451-160617, 160788-161009 |
| 400692 | 8118799 | Plus | 37906-38256 |
| 404224 | 6002294 | Minus | 80109-80957 |
| 405800 | 2791346 | Plus | 19271-19813 |
| 401149 | 7229925 | Plus | 73117-74019 |
| 405334 | 3135285 | Plus | 139386-139856 |
| 404958 | 7407941 | Minus | 2731-4531 |
| 405058 | 7655685 | Plus | 150740-151556 |
| 400663 | 8118474 | Plus | 88985-89281 |
| 403686 | 7387348 | Minus | 66625-68364 |
| 402399 | 1905915 | Minus | 24502-24666, 24986-25102 |
| 401624 | 8575907 | Plus | 168318-168444, 172964-173647 |
| 401706 | 4826475 | Minus | 14767-15317 |
| 405263 | 7329364 | Minus | 104082-104796 |
| 403796 | 8099896 | Minus | 75073-77664 |
| 405639 | 5091650 | Plus | 211184-211350 |
| 403794 | 8096910 | Plus | 163292-163884 |
| 403772 | 7770492 | Plus | 117668-117899 |
| 405126 | 8316124 | Plus | 45650-45911 |
| 400729 | 6705887 | Plus | 117048-118826 |
| 403243 | 7637828 | Minus | 76343-76585 |
| 402690 | 8348058 | Plus | 13368-13998 |
| 405677 | 4567140 | Plus | 31045-31445, 31688-31885 |
| 400522 | 9796828 | Plus | 40475-40792, 41517-41813, |
| | | | 42598-42894, 4325 |
| 406140 | 9168231 | Minus | 49887-50219 |
| 401371 | 9650602 | Plus | 80901-81283 |
| 406357 | 9256093 | Minus | 77181-77415 |
| 401554 | 8099284 | Minus | 153483-153612 |
| 405137 | 8570507 | Plus | 158969-159423 |
| 404843 | 7940338 | Plus | 22556-23222 |
| 403613 | 8493504 | Plus | 81290-81465 |
| 402534 | 9801061 | Plus | 58989-59457 |
| 401629 | 8575965 | Minus | 169336-169788 |
| 404731 | 7230299 | Minus | 168609-168781, 182951-183081 |
| 404219 | 1841541 | Plus | 50866-51013, 51517-51614, |
| | | | 51788-52030 |
| 405913 | 7712139 | Minus | 7484-7678 |
| 403180 | 7523976 | Minus | 63603-63759 |
| 401595 | 3293210 | Plus | 58192-58362 |
| 406089 | 9123919 | Plus | 73784-74296 |
| 401899 | 7230209 | Minus | 155620-155815 |
| 402137 | 7704985 | Plus | 11434-11799 |
| 404701 | 9800167 | Minus | 60061-60180 |
| 403474 | 9957921 | Plus | 55961-56511 |
| 405762 | 5931935 | Plus | 160502-161110 |
| 405590 | 6960456 | Plus | 90492-90818 |
| 404825 | 6478944 | Plus | 210382-210494 |
| 402638 | 9958128 | Minus | 82748-83226 |
| 406458 | 9756020 | Plus | 145874-146911 |
| 404923 | 7341893 | Plus | 98418-100223 |
| 403209 | 7630841 | Minus | 60664-60810, 85748-85939 |
| 400635 | 8567750 | Minus | 102800-102932, 107482-107689 |
| 402349 | 8191067 | Minus | 6817-6994, 7340-7680 |
| 402951 | 9408717 | Plus | 73252-73329, 73718-73877, |
| | | | 76217-76299, 7819 |
| 402447 | 9796640 | Plus | 47605-47729, 51696-51821, |
| | | | 52070-52257, 5330 |
| 403545 | 8078400 | Plus | 25293-25640 |
| 402561 | 9864675 | Plus | 72967-73163 |
| 401516 | 7637251 | Plus | 62335-62502 |
| 405691 | 4508112 | Plus | 171350-171739 |
| 404126 | 9796876 | Plus | 48919-49155 |
| 404046 | 3688074 | Minus | 2-773 |
| 402991 | 7631064 | Minus | 161294-161579 |
| 402320 | 7630359 | Minus | 46058-46153, 46446-46572 |
| 403227 | 7637789 | Plus | 24457-24771 |
| 401265 | 9797154 | Plus | 135413-135573 |
| 404247 | 7406725 | Minus | 83949-84214, 84312-84415, |
| | | | 84499-84677, 8487 |
| 404247 | 7406725 | Minus | 83949-84214, 84312-84415, |
| | | | 84499-84677, 8487 |
| 406370 | 9256130 | Plus | 125320-125482 |
| 404075 | 7652011 | Plus | 53692-53938 |
| 401703 | 4826475 | Plus | 135-1229 |
| 402252 | 3548788 | Plus | 6848-7049 |
| 402316 | 7527774 | Minus | 10751-10919, 18817-19052, |
| | | | 22131-22328 |
| 400818 | 8569994 | Plus | 172644-172765, 173085-173200 |
| 404900 | 7331453 | Plus | 22032-22219 |
| 400877 | 9857603 | Minus | 156938-157254 |
| 402610 | 9926549 | Minus | 22955-23124 |
| 404769 | 8099713 | Minus | 175801-176823 |
| 402863 | 2956660 | Minus | 29506-29693 |
| 402012 | 7407997 | Minus | 111771-111909, 112107-112226, |
| | | | 112519-11269 |
| 402411 | 9796294 | Minus | 109449-109598, 111328-111478 |
| 403567 | 8101141 | Plus | 35349-35614 |
| 401831 | 6850361 | Minus | 107181-108077 |
| 403248 | 7656833 | Minus | 167439-167606 |
| 402190 | 8576067 | Minus | 76488-76959 |
| 402878 | 9908870 | Minus | 56133-56522 |
| 401008 | 8117391 | Minus | 81421-81551, 82364-82512, |
| | | | 82862-82938 |
| 401037 | 7232177 | Minus | 3364-3686 |
| 404187 | 4481839 | Plus | 7644-7991 |
| 402869 | 6434643 | Minus | 138639-139335 |
| 404504 | 8151759 | Plus | 83050-83765 |
| 405593 | 6960456 | Plus | 157746-158046, 160760-160939 |
| 402255 | 3510233 | Plus | 9421-9621 |
| 406146 | 8746393 | Minus | 28514-29086 |
| 403722 | 7528070 | Minus | 117448-117615, 120977-121188 |
| 404805 | 4165236 | Minus | 37584-38304 |
| 403830 | 9887814 | Minus | 20687-20893 |
| 404909 | 7331453 | Minus | 123713-123909, 124327-124411, |
| | | | 124511-12462 |
| 401450 | 4680764 | Minus | 59252-59334, 59945-60427 |
| 405316 | 3638954 | Plus | 76171-76417, 76692-76844 |
| 404364 | 9964977 | Minus | 32986-33202 |
| 406138 | 9166422 | Plus | 42227-42753 |
| 402338 | 6957691 | Minus | 36915-37250 |
| 404244 | 5672609 | Minus | 98173-98517 |
| 402124 | 4033680 | Plus | 164206-164459 |
| 400470 | 9930824 | Minus | 156245-157227 |
| 406279 | 7547249 | Plus | 203752-204426 |
| 402635 | 9958100 | Minus | 167621-168379 |
| 401532 | 7798785 | Plus | 124414-124950, 125050-125418 |
| 403358 | 8570101 | Minus | 106778-107580 |
| 402517 | 9798106 | Plus | 17569-17721 |
| 400576 | 7923997 | Minus | 91711-91954 |
| 404907 | 7331453 | Minus | 102880-103828 |
| 400765 | 8131622 | Plus | 35000-35306 |
| 405201 | 7230116 | Plus | 36934-37314 |
| 405282 | 3810573 | Minus | 10482-10689 |
| 402022 | 7139714 | Plus | 165595-165748 |
| 403579 | 8101179 | Minus | 36167-36365 |
| 403957 | 8076835 | Minus | 81649-81754 |

Pkey: Unique number corresponding to an Eos probeset
Ref: Sequence source. The 7 digit numbers in this column are Genbank Identifier (GI) numbers. “Dunham, et al.” refers to the publication entitled “The DNA sequence of human chromosome 22” Dunham, et al. (1999) Nature 402: 489-495.
Strand: Indicates DNA strand from which exons were predicted.
Nt_position: Indicates nucleotide positions of predicted exons.

Table 13A lists about 102 genes up-regulated in Hepatitis C positive liver tissues from patients that are non-responsive to pegylated-interferon-alpha plus ribavirin treatment (non-responders) compared to Hepatitis C positive liver tissues from patients that are responsive to the treatment (responders). In both cases, the liver biopsies were obtained prior to treatment. These 102 genes have potential to be diagnostics and/or prognostic markers for determining the responsiveness of Hepatitis C infected liver tissues to pegylated-interferon-alpha plus ribavirin treatment. They may also provide clinical information on Hepatitis C infection and pathology. They may also be potential targets for therapeutic drugs and/or treatments. These were selected from 59680 probesets on the Affymetrix/Eos Hu03 GeneChip array such that the t-test p-value between the non-responders liver tissues group and the responders liver tissues group was less or equal to 0.01, and that the differences between the average “standardized values” of the different non-responders liver tissues was equal to or above 1.0 more than the average standardized values of the different responders liver tissues. The “standardized values” of the Hepatitis C liver tissues were derived by subtracting the median of the several different Hepatitis C negative liver tissue values from each probeset value, then divided by the interquantile range (IQR) of the same several Hepatitis C negative liver tissue values.

TABLE 13A

| Pkey | ExAccn | UniGene | Unigene Title | R1 | R2 |
|---|---|---|---|---|---|
| 440495 | AA887212 | Hs. 14161 | hypothetical protein DKFZp434l1930 | 4.81E−03 | 4.71 |
| 439242 | BE167324 | Hs. 53996 | ESTs, Weakly similar to ZN42_HUMAN ZINC | 1.71E−03 | 4.63 |
| 437855 | AA776740 | | ESTs | 2.88E−03 | 4.12 |
| 413321 | BE082938 | | gb: RC2-BT0642-290200-016-b02 BT0642 Homo | 9.87E−03 | 3.29 |
| 453696 | AI989482 | Hs. 146286 | kinesin family member 13A | 7.86E−03 | 3.05 |
| 452455 | N25153 | | ESTs, Weakly similar to AF174605 1 F-box | 5.32E−03 | 3.04 |
| 455348 | AW901517 | | gb: RC5-NN1013-310300-021-C03 NN1013 Homo | 3.47E−03 | 3.00 |
| 459577 | AA019148 | | gb: ze56e02.r1 Soares retina N2b4HR Homo | 5.46E−04 | 2.90 |
| 446633 | U85996 | Hs. 157142 | ESTs, Weakly similar to I38022 hypotheti | 6.87E−03 | 2.90 |
| 434848 | BE256304 | Hs. 32148 | AD-015 protein | 2.06E−04 | 2.78 |
| 452941 | AL110347 | Hs. 31074 | N-sulfoglucosamine sulfohydrolase (sulfa | 2.47E−03 | 2.76 |
| 407544 | Y10206 | | gb: H. sapiens mRNA for CD64 protein. | 2.28E−03 | 2.70 |
| 450578 | AI971773 | Hs. 232268 | ESTs | 3.64E−03 | 2.68 |
| 439774 | AL360257 | Hs. 213493 | Homo sapiens mRNA full length insert cDN | 4.81E−03 | 2.66 |
| 436427 | AI344378 | Hs. 143399 | ESTs | 4.46E−03 | 2.60 |
| 413348 | BE086846 | | gb: QV1-BT0678-300400-182-b06 BT0678 Homo | 2.15E−03 | 2.58 |
| 415210 | Z25274 | Hs. 154706 | hypothetical protein FLJ14917 | 8.96E−03 | 2.49 |
| 403512 | | | C3000579*: gi\|12643308\|sp\|Q9Y4K1\|AIM1_HUM | 8.08E−03 | 2.49 |
| 425225 | NM_003450 | Hs. 155204 | zinc finger protein 174 | 6.11E−03 | 2.49 |
| 422258 | AI910976 | | gb: wd22a06.x1 Soares_NFL_T_GBC_S1 Homo s | 5.28E−03 | 2.46 |
| 420400 | AI337097 | Hs. 156902 | ESTs | 5.73E−03 | 2.37 |
| 411729 | AW861879 | | gb: CM0-CT0341-260100-160-h12 CT0341 Homo | 2.55E−03 | 2.35 |
| 443933 | AI091631 | Hs. 203845 | two pore potassium channel KT3.3 | 2.27E−03 | 2.30 |
| 449137 | AW134478 | Hs. 196033 | ESTs | 8.66E−04 | 2.28 |
| 431498 | AK001777 | Hs. 258551 | aspartyl aminopeptidase | 4.39E−03 | 2.28 |
| 444811 | AW137791 | Hs. 148419 | ESTs | 1.97E−03 | 2.28 |
| 457365 | AA577297 | Hs. 303249 | EST | 4.58E−03 | 2.27 |
| 409198 | L19778 | Hs. 51011 | H2A histone family, member P | 1.64E−03 | 2.19 |
| 430144 | AI732722 | Hs. 187694 | ERGL protein; ERGIC-53-like protein | 3.29E−03 | 2.16 |
| 420803 | M91556 | Hs. 99945 | sodium channel, voltage-gated, type VI, | 6.76E−03 | 2.16 |
| 411504 | R90961 | Hs. 164584 | ESTs | 9.64E−03 | 2.07 |
| 428291 | AA534009 | Hs. 183487 | interferon stimulated gene (20 kD) | 2.28E−03 | 2.02 |
| 422211 | R84341 | Hs. 293845 | Homo sapiens, clone IMAGE: 3502329, mRNA, | 6.74E−03 | 2.00 |
| 453746 | AL120611 | Hs. 107924 | gb: DKFZp761H119_r1 761 (synonym: hamy2) | 1.78E−03 | 1.99 |
| 454278 | AF217525 | Hs. 49002 | Down syndrome cell adhesion molecule | 1.53E−03 | 1.96 |
| 447281 | AA017018 | Hs. 18021 | hypothetical protein FLJ20446 | 1.41E−03 | 1.94 |
| 452399 | BE513301 | Hs. 29344 | hypothetical protein, clone 24751 | 1.86E−03 | 1.94 |
| 426150 | NM_003658 | Hs. 167218 | BarH-like homeobox 2 | 6.19E−03 | 1.93 |
| 406773 | AA812424 | Hs. 76067 | heat shock 27 kD protein 1 | 1.01E−03 | 1.93 |
| 430259 | BE550182 | Hs. 127826 | RalGEF-like protein 3, mouse homolog | 4.72E−04 | 1.92 |
| 431368 | AA760776 | | ESTs | 1.90E−03 | 1.91 |
| 413201 | BE275378 | Hs. 13972 | hypothetical protein MGC12972 | 6.19E−03 | 1.91 |
| 408893 | AW605335 | Hs. 288826 | Homo sapiens cDNA FLJ12263 fis, clone MA | 8.42E−03 | 1.90 |
| 454521 | AW803516 | | gb: IL2-UM0081-230300-053-B12 UM0081 Homo | 6.83E−03 | 1.89 |
| 405419 | | | CX001144*: gi\|7242973\|dbj\|BAA92547.1\|(AB | 5.89E−03 | 1.88 |
| 403005 | | | C21000027*: gi\|1817556\|dbj\|BAA13672.1\|(D | 3.44E−03 | 1.84 |
| 454282 | AW296422 | | gb: UI-H-BW0-aio-h-05-0-UI.s1 NCI_CGAP_Su | 7.45E−03 | 1.84 |
| 449381 | AW388688 | Hs. 288217 | hypothetical protein MGC2941 | 7.54E−03 | 1.83 |
| 443305 | AI050693 | Hs. 133318 | ESTs | 9.26E−03 | 1.81 |
| 455154 | AW859359 | | gb: MR1-CT0353-150300-102-c02 CT0353 Homo | 8.62E−03 | 1.79 |
| 408172 | W02488 | Hs. 46039 | phosphoglycerate mutase 2 (muscle) | 9.27E−03 | 1.78 |
| 452711 | AW967047 | Hs. 293224 | ESTs, Weakly similar to T00375 hypotheti | 1.61E−03 | 1.70 |
| 448279 | BE250564 | Hs. 283655 | lysophospholipase II | 9.00E−03 | 1.67 |
| 408771 | AW732573 | Hs. 47584 | potassium voltage-gated channel, delayed | 3.00E−03 | 1.62 |
| 445909 | BE262656 | Hs. 32603 | hypothetical protein MGC3279 similar to | 6.81E−03 | 1.61 |
| 445058 | AA234031 | Hs. 326104 | putative mitochondrial solute carrier | 8.61E−03 | 1.60 |
| 442620 | C00138 | Hs. 8535 | Homo sapiens mRNA for KIAA1668 protein, | 8.16E−03 | 1.59 |
| 431369 | BE184455 | Hs. 251754 | secretory leukocyte protease inhibitor ( | 1.85E−03 | 1.58 |
| 441307 | AW071696 | Hs. 209065 | hypothetical protein FLJ14225 | 1.73E−04 | 1.56 |
| 435012 | AA768359 | Hs. 149452 | ESTs | 7.59E−03 | 1.55 |
| 447606 | AI588954 | Hs. 170995 | ESTs | 7.61E−03 | 1.55 |
| 432911 | AW807634 | Hs. 279799 | putative zinc finger protein NY-REN-34 a | 9.38E−03 | 1.54 |
| 450812 | AB002360 | Hs. 25515 | MCF.2 cell line derived transforming seq | 2.25E−03 | 1.52 |
| 441164 | AB023180 | Hs. 7724 | KIAA0963 protein | 7.60E−03 | 1.52 |
| 455749 | BE075035 | | gb: PM3-BT0584-260300-002-g05 BT0584 Homo | 2.33E−03 | 1.51 |
| 412732 | AW993300 | | gb: RC2-BN0033-180200-015-g06 BN0033 Homo | 4.33E−03 | 1.49 |
| 414056 | AI187349 | Hs. 17681 | ESTs | 6.51E−03 | 1.49 |
| 458765 | AI419235 | Hs. 344456 | gb: tf21d02.x1 NCI_CGAP_Brn23 Homo sapien | 2.19E−03 | 1.44 |
| 407496 | U20648 | | gb: Human zinc finger protein (ZNF154) mR | 6.69E−03 | 1.42 |
| 434415 | BE177494 | | gb: RC6-HT0596-270300-011-C05 HT0596 Homo | 6.67E−03 | 1.39 |
| 407233 | X16354 | Hs. 50964 | carcinoembryonic antigen-related cell ad | 8.97E−03 | 1.38 |
| 410020 | T86315 | Hs. 728 | ribonuclease, RNase A family, 2 (liver, | 4.92E−03 | 1.37 |
| 454303 | AW857169 | | gb: RC1-CT0302-110500-018-f10 CT0302 Homo | 3.89E−03 | 1.34 |
| 401411 | | | ENSP00000247172*: HYPOTHETICAL 126.2 kDa | 5.91E−03 | 1.33 |
| 444754 | T83911 | | transmembrane 4 superfamily member 4 | 6.61E−04 | 1.32 |
| 446411 | AI298828 | Hs. 153439 | ESTs | 1.63E−03 | 1.30 |
| 428407 | NM_003963 | Hs. 184194 | Transmembrane 4 superfamily member 5 | 5.64E−04 | 1.30 |
| 452910 | AI927371 | Hs. 288839 | hypothetical protein FLJ12178 | 9.89E−03 | 1.29 |
| 435791 | AA243086 | Hs. 25204 | chondroitin 4-O-sulfotransferase 2 | 4.10E−03 | 1.29 |
| 441504 | BE159181 | Hs. 168232 | hypothetical protein FLJ13855 | 5.78E−03 | 1.29 |
| 456477 | BE550252 | Hs. 1295 | calcium channel, voltage-dependent, alph | 9.15E−03 | 1.28 |
| 446388 | AA292979 | Hs. 7788 | NPD007 protein | 4.50E−03 | 1.27 |
| 418863 | AL135743 | Hs. 25566 | ESTs, Weakly similar to 2004399A chromos | 8.80E−03 | 1.24 |
| 406684 | X16354 | Hs. 50964 | carcinoembryonic antigen-related cell ad | 9.58E−04 | 1.24 |
| 414784 | NM_000344 | Hs. 288986 | survival of motor neuron 1, telomeric | 7.19E−03 | 1.24 |
| 425429 | U18009 | Hs. 157236 | membrane protein of cholinergic synaptic | 8.57E−03 | 1.22 |
| 441252 | AW360901 | | hypothetical protein MGC4399 | 6.93E−03 | 1.21 |
| 448222 | AI648587 | Hs. 20725 | Mov10 (Moloney leukemia virus 10, mouse) | 4.64E−03 | 1.21 |
| 414395 | BE304888 | Hs. 279834 | EST | 6.16E−03 | 1.20 |
| 441244 | BE612935 | Hs. 184052 | PP1201 protein | 2.70E−03 | 1.19 |
| 420886 | AA805453 | | ESTs, Weakly similar to T29012 hypotheti | 9.38E−03 | 1.18 |
| 444972 | AW195642 | Hs. 148901 | ESTs | 7.68E−03 | 1.18 |
| 417418 | NM_002468 | Hs. 82116 | myeloid differentiation primary response | 9.64E−03 | 1.18 |
| 427672 | AA356615 | Hs. 336916 | death-associated protein 6 | 1.92E−04 | 1.17 |
| 451025 | AW028689 | Hs. 301985 | ESTs | 9.53E−03 | 1.16 |
| 407195 | C21124 | | gb: HUMGS0002072 Human adult (K. Okubo) Ho | 1.84E−03 | 1.14 |
| 437226 | AA432308 | | Homo sapiens mRNA; cDNA DKFZp434M0420 (f | 5.73E−03 | 1.13 |
| 407465 | AJ401466 | | gb: Homo sapiens mRNA for high affinity c | 2.51E−03 | 1.12 |
| 444751 | AI207406 | Hs. 11866 | translocase of inner mitochondrial membr | 8.36E−03 | 1.12 |
| 429396 | AW954598 | | Homo sapiens clone 25015 mRNA sequence | 5.46E−03 | 1.06 |
| 457097 | AB037727 | Hs. 334479 | hypothetical protein DKFZp586I021 | 9.69E−03 | 1.05 |
| 440464 | AI917706 | Hs. 129997 | ESTs | 7.61E−03 | 1.02 |

Pkey: Unique Eos probeset identifier number
ExAccn: Exemplar Accession number, Genbank accession number
UnigeneID: Unigene number
Unigene Title: Unigene gene title
R1: T-test p-value
R2: Difference of the average standardized value of non-responders vs. the average standardized value of responders

TABLE 13B

| Pkey | CAT Number | Accession |
|---|---|---|
| 437855 | 36765_2 | AK056396 AI800037 AA777098 BF939153 AI621113 AA776740 |
| | | BF446127 AW295790 AA769865 AI690256 N32072 H55081 AL044298 AI286018 |
| | | BI044678 N45384 |
| 413321 | 1501922_1 | BE083171 BE082893 BE082938 |
| 452455 | 928588_1 | BE877195 BG988071 AI902576 N25153 AW518714 N25154 |
| 455348 | 1159211_1 | AW901523 AW901517 BF363877 BF363878 AW901521 AW901547 |
| 459577 | 86256_1 | W31316 AA019148 BF921026 BF921163 |
| 413348 | 1508017_1 | BE086846 BE086726 BE086733 BE086849 BE086683 |
| 422258 | 868626_1 | AW957794 AW957873 AA307574 AI910976 AA481204 |
| 411729 | 1099453_1 | AW861879 AW861948 AW858447 AW861873 AW858418 AW861871 |
| 431368 | 331947_1 | AW969020 AA760776 AA504117 AA731957 AA504375 BF962819 |
| 454521 | 1058686_1 | BE154783 AW803516 AW803639 |
| 454282 | 725499 _1 | H72616 H63825 AW296422 |
| 455154 | 1100782_1 | AW859359 AW859373 AW859358 AW859368 AW859363 AW859370 AW859347 |
| 455749 | 1497012_1 | BE075035 BE074999 BE075006 BE075008 BE075005 BE075032 BE075037 |
| 412732 | 1245294_1 | AW993300 R22345 N23107 |
| 434415 | 110905_1 | BE177494 AW276909 AA632849 |
| 454303 | 306545_1 | AW857169 BF929104 AW857162 BE153976 BE141977 AW352163 BE141954 |
| 444754 | 2600_1 | AK055577 U31449 NM_004617 BC001386 BE617218 BE868856 BM264336 |
| | | BG385990 BM353313 BI439041 BM272005 BM314705 BG616897 |
| | | AA345452 BM314731 AA345379 AA344984 BG191629 BF002707 BE138874 AI676255 AI675920 BM264034 |
| | | BI438713 BM314426 AW471424 |
| | | BF063338 BM311530 AW518986 BM314400 AI572072 AI337361 AW013983 AI569109 AW338459 |
| | | AA452917 AI572053 AI886493 R07511 |
| | | AA506545 AA480323 AA908761 AI921215 R11516 AI583063 AI584071 AW339205 AI640782 R85697 |
| | | AI784149 AI584060 AI566443 AI982840 |
| | | BI439748 H78445 H94341 BG210200 BI466960 BG197730 H59265 BG821834 AW951290 R96709 H90091 |
| | | T83911 T68523 R07564 |
| 441252 | 122_1 | NM_032315 BC004991 BM468629 AL583162 BE741793 BE303031 BE244077 |
| | | BE302759 AL583161 AL572691 AI804708 AI129827 AW872388 |
| | | AI653103 AI151487 BE504355 AI671476 AW770976 AI206149 AI678224 BE468167 AI201164 AI200893 |
| | | AA574128 AI991968 AW194793 |
| | | AA426113 AI220474 AI201028 AA757281 AA844723 AA398466 AA484977 BF059403 AI470557 AA435830 |
| | | AW016602 AI915990 BF356355 |
| | | AL569855 AW978643 AW242882 AI823516 AW235374 BF059684 C20622 T66303 AI912746 AW339765 |
| | | AA968541 AL526958 BF433829 |
| | | AA628313 AW578403 AI909734 N75265 AW274422 W02124 BG474952 |
| 420886 | 56595_1 | AK057268 AA281379 BI856491 AA805453 AA281414 BF373287 |
| 407195 | 1786254_1 | AA569403 C21124 |
| 437226 | 2332_1 | AL117642 BF056754 AI952151 AI953773 AL045835 AI041069 BF765693 BF765688 |
| | | BF763048 BG682692 AW967083 BM271900 AV704704 |
| | | AL040396 AI627368 AW514803 AI761270 AI015622 AI862541 AW197157 AI697779 AI568646 AI200936 |
| | | BE695042 AI366513 BE711314 |
| | | BE711324 BE047438 AW474529 AI421029 AL046814 AA400447 BF941647 AI143869 AW591727 BE695050 |
| | | BF887502 AW963918 BF001425 |
| | | AI340187 AW006934 AW468251 AW467398 AW516879 BF871799 AI816703 AI223153 BM264535 |
| | | AI624845 BE711307 AW514703 AW264118 |
| | | AW474488 BM263438 AW204628 BE711312 BE711330 AI718586 AI247201 BE711341 BF593534 AA626326 |
| | | AA747785 AA782029 AI341349 |
| | | AI921814 AW613873 BF332890 BE244575 BE694998 BF197632 AA609664 AW235441 BE042387 |
| | | AA704683 AA758933 AI970214 BI025187 |
| | | AA432308 BF871801 AW008470 BF765466 BE711329 BE711328 BF765463 BF765739 AW129408 |
| | | BF765015 BF764200 AL042362 H58451 |
| | | T86447 AA873427 AA570077 AW771950 AA400603 N69252 H16763 T65234 AA992089 AA836232 |
| | | AA813701 AA026623 W80716 AA861629 |
| | | N54181 N54185 AI015075 AA236563 R54957 |
| 407465 | 303_1 | AB043997 NM_021815 AJ401466 AF276871 AV709773 AA364959 |
| | | AA363955 AA364965 |
| 429396 | 1752_1 | AK056027 AL120636 AA582324 AA582305 AI459497 BI047007 AL589306 AI688987 |
| | | AI922469 AA350721 AI085972 AF131844 AW954598 |
| | | AW957578 AA350710 AI884831 R37119 AA666294 AA350709 BF724097 AA203665 AI089250 AA708285 |
| | | BF831652 AW104744 AW104745 |
| | | AW129091 AV724110 AW805934 AV682207 AA563965 W93840 AW104743 AW009432 AI085993 BF839379 |
| | | T79879 BC019656 AA975816 |
| | | AI953836 BE350274 AW840298 BI043880 BG913327 AW839960 T07306 R53951 AI299256 AI961918 |
| | | AI190874 AA724491 F04512 BG997879 |
| | | R08567 BE810546 |

Pkey: Unique Eos probeset identifier number
CAT number: Gene cluster number
Accession: Genbank accession numbers

TABLE 13C

| Pkey | Ref | Strand | Nt_position |
|---|---|---|---|
| 403512 | 7656757 | Minus | 114487-114610 |
| 405419 | 4753290 | Minus | 60924-61120, 61641-61773 |
| 403005 | 5791501 | Minus | 16945-17053, 20018-20403 |
| 401411 | 7799787 | Minus | 144144-144329 |

Pkey: Unique number corresponding to an Eos probeset
Ref: Sequence source. The 7 digit numbers in this column are Genbank Identifier (GI) numbers. “Dunham, et al.” refers to the publication entitled “The DNA sequence of human chromosome 22” Dunham, et al. (1999) Nature 402: 489-495.
Strand: Indicates DNA strand from which exons were predicted.
Nt_position: Indicates nucleotide positions of predicted exons.

Table 14A lists about 147 genes up-regulated in Hepatitis C positive liver tissues from patients that are responsive to pegylated-interferon-alpha plus ribavirin treatment (responders) compared to Hepatitis C positive liver tissues from patients that are not responsive to the treatment (non-responders). In both cases, the liver biopsies were obtained prior to treatment. These 147 genes have the potential to be diagnostics and/or prognostic markers for determining the responsiveness of Hepatitis C infected liver tissues to pegylated-interferon-alpha plus ribavirin treatment They may also provide clinical information on Hepatitis C infection and pathology. These were selected from 59680 probesets on the Affymetrix/Eos Hu03 GeneChip array such that the t-test p-value between the non-responders liver tissues group and the responders liver tissues group was less or equal to 0.01, and that the differences between the average “standardized values” of the several different responders liver tissues was equal to or above 1.0 more than the average standardized values of the several different non-responders liver tissues. The “standardized values” of the Hepatitis C liver tissues were derived by subtracting the median of the several different Hepatitis C negative liver tissue values from each probeset value, then divided by the interquantile range (IQR) of the same several Hepatitis C negative liver tissue values.

TABLE 14A

| Pkey | ExAccn | UnigeneID | Unigene Title | R1 | R2 |
|---|---|---|---|---|---|
| 413278 | BE563085 | Hs. 833 | interferon-stimulated protein, 15 kDa | 2.39E−04 | 8.04 |
| 402399 | | | C19001025*: gi\|11275298\|dbj\|BAB18302.1\|( | 9.60E−03 | 4.25 |
| 446783 | AW138343 | Hs. 141867 | ESTs | 2.95E−03 | 4.19 |
| 422596 | AF063611 | Hs. 118633 | 2′-5′-oligoadenylate synthetase-like | 5.19E−03 | 4.03 |
| 433364 | AI075407 | Hs. 296083 | ESTs, Moderately similar to I54374 gene | 6.90E−03 | 3.97 |
| 447567 | AW474513 | Hs. 224397 | ESTs, Weakly similar to I38931 Wiskott-A | 6.87E−03 | 3.89 |
| 432680 | T47364 | Hs. 278613 | interferon, alpha-inducible protein 27 | 4.46E−03 | 3.28 |
| 457974 | AW842353 | Hs. 321717 | ESTs, Weakly similar to S22765 heterogen | 9.02E−03 | 2.76 |
| 448436 | AI520706 | Hs. 171012 | hypothetical protein FLJ22349 | 3.22E−03 | 2.71 |
| 405481 | | | Target Exon | 7.36E−03 | 2.65 |
| 415682 | AI347128 | Hs. 191870 | ESTs | 6.94E−03 | 2.64 |
| 444534 | AW271626 | Hs. 42294 | ESTs | 3.28E−03 | 2.62 |
| 417199 | H48304 | | gb: yq77c01.r1 Soares fetal liver spleen | 2.23E−03 | 2.50 |
| 440798 | AB020648 | Hs. 7426 | KIAA0841 protein | 1.00E−03 | 2.40 |
| 458492 | AI143655 | | ESTs | 8.29E−03 | 2.27 |
| 453989 | M63962 | Hs. 36992 | ATPase, H? exchanging, alpha polypeptide | 1.16E−03 | 2.25 |
| 409703 | NM_006187 | Hs. 56009 | 2′-5′-oligoadenylate synthetase 3 (100 k | 5.99E−03 | 2.22 |
| 412003 | R11775 | | ESTs, Weakly similar to Z195_HUMAN ZINC | 7.01E−03 | 2.20 |
| 400423 | AF264765 | | Target | 7.03E−03 | 2.16 |
| 409665 | NM_006731 | Hs. 55777 | Fukuyama type congenital muscular dystro | 8.24E−03 | 2.12 |
| 438146 | Z36842 | Hs. 57548 | ESTs | 9.92E−03 | 2.10 |
| 401191 | | | crystallin, beta A1 | 6.98E−03 | 2.06 |
| 420875 | AA782222 | Hs. 293224 | ESTs, Weakly similar to ALU1_HUMAN ALU S | 5.64E−03 | 2.06 |
| 423729 | R01566 | Hs. 5461 | ESTs | 1.99E−03 | 2.04 |
| 400301 | X03635 | Hs. 1657 | estrogen receptor 1 | 2.35E−03 | 2.03 |
| 402885 | | | Target Exon | 4.26E−03 | 2.00 |
| 431620 | AA126109 | Hs. 264981 | 2′-5′-oligoadenylate synthetase 2 (69-71 | 4.05E−03 | 1.96 |
| 414915 | NM_002462 | Hs. 76391 | myxovirus (influenza) resistance 1, homo | 2.34E−03 | 1.94 |
| 435956 | AF269255 | Hs. 22604 | lysosomal apyrase-like protein 1 | 1.31E−03 | 1.90 |
| 418165 | R45959 | Hs. 6637 | ESTs | 3.05E−03 | 1.89 |
| 449261 | AI637592 | | ESTs | 3.00E−03 | 1.89 |
| 458833 | AW236702 | Hs. 171431 | ESTs, Weakly similar to A46010 X-linked | 6.83E−03 | 1.87 |
| 433156 | R59206 | Hs. 17519 | Homo sapiens cDNA: FLJ22539 fis, clone H | 4.62E−04 | 1.86 |
| 405468 | | | NM_022820*: Homo sapiens cytochrome P450 | 1.23E−03 | 1.85 |
| 420772 | AW752656 | Hs. 222707 | KIAA1718 protein | 3.77E−04 | 1.85 |
| 451505 | AI290219 | Hs. 25223 | ESTs, Highly similar to YSHUT threonine- | 7.46E−03 | 1.84 |
| 423955 | AI420582 | Hs. 136164 | cutaneous T-cell lymphoma-associated tum | 7.60E−04 | 1.83 |
| 433207 | AB040903 | Hs. 284146 | hypothetical protein DKFZp762N0610 | 9.89E−03 | 1.83 |
| 402605 | | | Target Exon | 3.64E−03 | 1.82 |
| 427958 | AA418000 | Hs. 98280 | potassium intermediate/small conductance | 5.64E−03 | 1.82 |
| 416879 | H98899 | Hs. 42599 | ESTs | 8.76E−03 | 1.81 |
| 458891 | AI659166 | Hs. 207144 | ESTs | 8.31E−03 | 1.78 |
| 432384 | AW006770 | Hs. 162193 | ESTs | 4.60E−03 | 1.78 |
| 424049 | A8014524 | Hs. 138380 | KIAA0624 protein | 3.61E−04 | 1.77 |
| 415723 | AA330633 | Hs. 23450 | mitochondrial ribosomal protein S25 | 2.35E−03 | 1.76 |
| 437556 | AI223078 | Hs. 136776 | ESTs | 4.24E−03 | 1.75 |
| 436869 | NM_014867 | Hs. 5333 | KIAA0711 gene product | 6.34E−03 | 1.75 |
| 417054 | AF017060 | | aldehyde oxidase 1 | 3.52E−03 | 1.75 |
| 411194 | AW821559 | | gb: IL2-ST0311-271299-030-D03 ST0311 Homo | 4.86E−03 | 1.74 |
| 421822 | AV650066 | Hs. 1430 | coagulation factor XI (plasma thrombopla | 9.90E−04 | 1.71 |
| 453866 | AW291498 | Hs. 250557 | ESTs | 6.34E−03 | 1.70 |
| 414812 | X72755 | Hs. 77367 | monokine induced by gamma interferon | 2.10E−03 | 1.68 |
| 457247 | AA458605 | | KIAA1681 protein | 9.08E−03 | 1.66 |
| 426315 | AA854219 | Hs. 348137 | Homo sapiens, clone IMAGE: 3542716, mRNA, | 1.33E−03 | 1.66 |
| 449774 | R06076 | | gb: ye94c02.s1 Soares fetal liver spleen | 1.81E−03 | 1.66 |
| 439281 | AA100768 | Hs. 48485 | ESTs, Weakly similar to I38022 hypotheti | 9.06E−03 | 1.65 |
| 435545 | AA687415 | Hs. 28107 | ESTs | 5.39E−03 | 1.65 |
| 453336 | AW139766 | Hs. 233431 | ESTs | 5.32E−04 | 1.64 |
| 403640 | | | ENSP00000233023*: CDNA FLJ12662 fis, clon | 8.54E−04 | 1.63 |
| 436790 | AI498610 | | ESTs | 5.14E−03 | 1.62 |
| 400552 | | | Target Exon | 9.71E−04 | 1.62 |
| 427185 | AA398930 | | ESTs | 3.78E−03 | 1.61 |
| 403609 | | | C3001199: gi\|7494834\|pir\|\|T15308 hypothet | 1.26E−03 | 1.60 |
| 425468 | BE141170 | | gb: MR0-HT0077-011099-001-a05 HT0077 Homo | 5.35E−03 | 1.59 |
| 415374 | F06904 | Hs. 8346 | ESTs | 8.79E−03 | 1.59 |
| 440700 | AW952281 | Hs. 296184 | guanine nucleotide binding protein (G pr | 9.04E−03 | 1.59 |
| 424465 | AI216391 | Hs. 169939 | heparan sulfate 2-O-sulfotransferase | 3.85E−03 | 1.56 |
| 430374 | AF064104 | Hs. 22116 | CDC14 (cell division cycle 14, S. cerevi | 1.66E−03 | 1.56 |
| 418129 | X52997 | Hs. 1144 | glycoprotein IX (platelet) | 8.20E−03 | 1.55 |
| 421497 | BE145334 | | BANP homolog, SMAR1 homolog | 8.31E−04 | 1.54 |
| 440083 | AI433721 | Hs. 164153 | ESTs | 4.34E−03 | 1.53 |
| 401740 | | | ENSP00000005503*: SA (rat hypertension-as | 8.69E−03 | 1.53 |
| 447083 | AI472124 | Hs. 157757 | ESTs | 7.48E−03 | 1.52 |
| 411183 | BE141178 | | gb: MR0-HT0077-011099-002-b06 HT0077 Homo | 6.03E−03 | 1.52 |
| 426912 | AL043054 | Hs. 256657 | ESTs, Weakly similar to A46302 PTB-assoc | 2.41E−03 | 1.51 |
| 405720 | | | Target Exon | 5.64E−03 | 1.51 |
| 442068 | BE312873 | Hs. 314932 | ESTs | 5.81E−03 | 1.50 |
| 400279 | | | NM_004581*: Homo sapiens Rab geranylgeran | 9.72E−03 | 1.49 |
| 428138 | AA773842 | Hs. 293799 | ESTs | 2.77E−03 | 1.49 |
| 451792 | AA019795 | Hs. 200235 | ESTs | 1.40E−03 | 1.49 |
| 400846 | | | sortilin-related receptor, L(DLR class) | 7.96E−04 | 1.48 |
| 438683 | AA813982 | | ESTs | 7.91E−03 | 1.48 |
| 425304 | AA463844 | Hs. 31339 | fibroblast growth factor 11 | 9.32E−03 | 1.42 |
| 433785 | BE044593 | Hs. 112704 | ESTs | 3.60E−03 | 1.42 |
| 421488 | F13451 | Hs. 77515 | gb: HSC37A091 normalized infant brain cDN | 4.59E−03 | 1.41 |
| 443150 | AI034467 | Hs. 34650 | ESTs | 4.28E−04 | 1.39 |
| 401439 | | | NM_022495*: Homo sapiens hypothetical pro | 9.26E−04 | 1.38 |
| 429443 | AB028967 | Hs. 202687 | potassium voltage-gated channel, Shal-re | 3.53E−03 | 1.36 |
| 408299 | AW886716 | Hs. 154396 | hypothetical protein FLJ22282 | 3.26E−03 | 1.35 |
| 427009 | AA394290 | Hs. 97353 | ESTs | 5.89E−03 | 1.34 |
| 415770 | M79237 | | gb: EST01385 Subtracted Hippocampus, Stra | 3.83E−03 | 1.34 |
| 431718 | AI337294 | | GaINAc alpha-2, 6-sialyltransferase I, I | 8.31E−04 | 1.33 |
| 443765 | AI084960 | Hs. 334589 | ESTs | 1.23E−03 | 1.33 |
| 436945 | AA918210 | Hs. 121116 | ESTs | 6.32E−03 | 1.33 |
| 429018 | AW892660 | Hs. 146286 | kinesin family member 13A | 8.65E−04 | 1.33 |
| 428207 | BE263644 | Hs. 169119 | gb: 601192069F1 NIH_MGC_7 Homo sapiens cD | 3.47E−03 | 1.30 |
| 433611 | AW327692 | Hs. 3446 | mitogen-activated protein kinase kinase | 5.80E−03 | 1.29 |
| 438900 | AA827856 | Hs. 124299 | ESTs | 9.32E−03 | 1.29 |
| 444131 | AI806600 | Hs. 328579 | EST, Weakly similar to A55362 procollage | 6.31E−03 | 1.28 |
| 458153 | AI792267 | Hs. 125363 | ESTs | 2.19E−03 | 1.28 |
| 425326 | AW501870 | | gb: UI-HF-BR0p-ajo-g-06-0-UI.r1 NIH_MGC_5 | 4.51E−03 | 1.28 |
| 428803 | AA913233 | Hs. 153179 | ESTs, Highly similar to Human Recombinan | 7.01E−03 | 1.28 |
| 409570 | AW418720 | Hs. 167583 | ESTs | 7.21E−03 | 1.26 |
| 415835 | Z45365 | | gb: HSC2NF061 normalized infant brain cDN | 5.71E−03 | 1.26 |
| 403740 | | | NM_001076*: Homo sapiens UDP glycosyltran | 4.08E−03 | 1.24 |
| 446780 | R31107 | | gb: yh61g01.s1 Soares placenta Nb2HP Homo | 2.41E−03 | 1.24 |
| 443773 | AV646452 | Hs. 30941 | calcium channel, voltage-dependent, beta | 4.95E−03 | 1.24 |
| 409008 | AA059392 | Hs. 66791 | ESTs | 6.61E−03 | 1.23 |
| 426426 | AI569804 | Hs. 42792 | ESTs, Weakly similar to I78885 serine/th | 6.82E−03 | 1.23 |
| 436752 | AW298529 | | ESTs | 2.55E−03 | 1.23 |
| 455202 | AW865320 | | gb: PM4-SN0020-300300-007-a11 SN0020 Homo | 8.34E−03 | 1.21 |
| 445613 | BE550889 | Hs. 158491 | ESTs | 7.33E−03 | 1.20 |
| 442387 | AI239835 | Hs. 341782 | ESTs | 2.43E−03 | 1.19 |
| 444521 | H58280 | Hs. 8366 | ESTs | 4.22E−04 | 1.19 |
| 458273 | AW770493 | Hs. 182874 | guanine nucleotide binding protein (G pr | 9.33E−03 | 1.19 |
| 419662 | W87674 | | gb: zh66a01.r1 Soares_fetal_liver_spleen_{—} | 6.47E−04 | 1.18 |
| 402842 | | | ENSP00000241325*: DJ947L8.1.3 (novel CUB | 7.14E−03 | 1.18 |
| 417715 | AW969587 | Hs. 86366 | ESTs | 9.63E−03 | 1.15 |
| 410209 | AI583661 | Hs. 60548 | hypothetical protein PRO1635 | 3.03E−03 | 1.15 |
| 414832 | W27128 | Hs. 100057 | Homo sapiens cDNA: FLJ22902 fis, clone K | 1.12E−03 | 1.14 |
| 425851 | NM_001490 | Hs. 159642 | glucosaminyl (N-acetyl) transferase 1, c | 7.36E−03 | 1.14 |
| 408906 | AW294539 | Hs. 255323 | ESTs | 7.49E−03 | 1.13 |
| 444211 | AW390415 | Hs. 79013 | ESTs, Moderately similar to 1207289A rev | 5.07E−03 | 1.13 |
| 444362 | AW892056 | Hs. 110853 | ESTs | 2.18E−03 | 1.12 |
| 418453 | T93674 | Hs. 268815 | ESTs | 8.59E−03 | 1.11 |
| 427761 | AA412205 | Hs. 140996 | ESTs | 8.41E−03 | 1.11 |
| 402149 | | | Target Exon | 6.08E−03 | 1.10 |
| 412150 | AW895719 | | gb: QV4-NN0039-290300-154-f06 NN0039 Homo | 2.46E−03 | 1.10 |
| 446250 | AI283067 | Hs. 320873 | EST | 9.30E−03 | 1.10 |
| 427201 | AB037860 | Hs. 173933 | nuclear factor I/A | 2.60E−03 | 1.10 |
| 439620 | AA838727 | Hs. 124405 | ESTs, Weakly similar to A46010 X-linked | 6.98E−03 | 1.10 |
| 401929 | | | C17001690: gi\|6005701\|ref\|NP_009099.1\|AT | 8.77E−03 | 1.08 |
| 406466 | | | Target Exon | 4.76E−04 | 1.07 |
| 446266 | AI417271 | Hs. 163949 | ESTs, Weakly similar to I38022 hypotheti | 5.84E−03 | 1.06 |
| 424579 | BE614288 | Hs. 285391 | DKFZP564P1916 protein | 4.35E−03 | 1.06 |
| 453585 | AL043430 | Hs. 325309 | hypothetical protein FLJ14596 | 3.07E−03 | 1.06 |
| 420399 | AA743258 | Hs. 291545 | ESTs | 6.92E−03 | 1.05 |
| 435147 | AL133731 | Hs. 4774 | Homo sapiens mRNA; cDNA DKFZp761C1712 (f | 6.17E−03 | 1.04 |
| 400636 | | | C10001873: gi\|11056014\|ref\|NP_067651.1\|a | 8.97E−03 | 1.03 |
| 436737 | AI052229 | Hs. 25373 | ESTs, Weakly similar to T20410 hypotheti | 8.30E−03 | 1.03 |
| 414278 | AA330116 | | Human glucose transporter pseudogene | 2.95E−03 | 1.02 |
| 457599 | AW997833 | Hs. 257267 | FYVE and coiled-coil domain containing 1 | 5.05E−03 | 1.01 |
| 431006 | BE152871 | | gb: CM1-HT0333-101299-064-d12 HT0333 Homo | 5.72E−03 | 1.01 |
| 421932 | W51778 | Hs. 323949 | kangai 1 (suppression of tumorigenicity | 2.60E−03 | 1.01 |
| 405761 | | | ENSP00000203606: CDNA FLJ14191 fis, clone | 6.05E−03 | 1.01 |
| 450078 | AI681743 | | gb: tx38g10.x1 NCI_CGAP_Lu24 Homo sapiens | 3.38E−03 | 1.01 |
| 402942 | | | Target Exon | 5.84E−03 | 1.00 |

Pkey: Unique Eos probeset identifier numbe
ExAccn: Exemplar Accession number, Genbank accession number
UnigeneID: Unigene number
Unigene Title: Unigene gene title
R1: T-test p-value
R2: Difference of the average standardized value of responders vs. the average standardized value of non-responders

TABLE 14B

| Pkey | CAT Number | Accession |
|---|---|---|
| 417199 | 1965264_1 | AA194635 H48304 R22482 R99998 |
| 458492 | 2797412_1 | AI869287 AI143655 |
| 412003 | 1146633_1 | R11775 F05441 F07670 AW883427 T26374 |
| 449261 | 3029652_1 | AI923721 AI860294 AI637592 |
| 417054 | 12405_2 | BG533564 BG618564 AW296119 AI269233 BF508328 AW364777 AW292258 |
| | | AA371049 AI452471 AI092522 BG618376 AL049080 AA631068 |
| | | BG564643 T53833 AV702544 BG533452 AV705004 AA588281 T28665 BG569026 AV646874 AV647253 |
| | | AV647455 AV647749 BI759444 |
| | | AV652457 AV695354 AV696010 AV697248 BG617586 AV722549 AI435836 AI590676 AI245019 AW338243 |
| | | AA530898 D52191 AI435352 |
| | | D57473 BG566952 AI420505 AA035245 AV704972 BG564113 AI439237 AI287456 AV695686 AA349017 |
| 411194 | 1072676_1 | AW821558 AW821559 AW821557 AW821643 AW821560 AW821497 AW821639 |
| | | AW821498 AW821496 AW821499 |
| 457247 | 22785_1 | AB051468 AW977252 AI261627 AW665579 AW274550 AI418272 AW293861 |
| | | AA731376 BF798878 BF445807 AB053320 AK022729 AW959911 |
| | | AV658411 H10450 AI073358 AA889598 BF445392 AI368772 AA421057 AA702576 F09058 AA327610 |
| | | AL135425 AU135871 AU125838 |
| | | BE467087 BG661269 AI697843 BF111090 AI814556 AI689772 BE348775 AI689791 BF475299 AA987614 |
| | | AI802005 N79670 BG564339 |
| | | AW172331 AW592078 AA487432 AI270312 BF062573 AI755254 AI655337 AI753287 AI753432 N63992 |
| | | AA758915 AA903180 AA126824 |
| | | AI202889 AA778528 H99263 BF435656 BI755650 AW806648 BE547432 AA872493 H49166 AI167329 |
| | | BG222086 AW751006 AW751005 |
| | | BE827287 BI521573 BE737978 AA194149 W74616 AW138578 AI128240 AI498242 BM314911 |
| | | AA642425 AW583264 AI332987 AW241875 |
| | | N62879 AI362749 AI761951 AI370777 AI332423 AA903009 AI207967 AI435328 BG222922 BG222858 |
| | | AW262522 AA758655 AA649251 |
| | | AA235233 N77786 H30532 H30545 T94316 AW855131 AW992588 BG998544 BE076575 BG958225 |
| | | AW772819 T83121 T80942 BF088923 |
| 449774 | 79702_1 | AA007258 R06179 AA004224 R06076 |
| 436790 | 647116_1 | AI936636 AI498610 AI589571 AA731428 AW182143 |
| 427185 | 1312946_1 | AI204518 AA398930 AA402069 |
| 425468 | 1280868_1 | BE141170 BE141413 BE141342 AA357975 |
| 421497 | MH1660_34 | BM012343 BG990287 BI054221 AA292029 BE090740 BE147105 BF945352 |
| | | BE147159 AW837076 BE702303 BE166423 BF833717 BF898447 |
| | | BF945348 AW085137 N58701 T81141 BG977546 BE160737 R81151 BF833715 R76844 BE145334 |
| | | AA292072 BF747231 |
| 411183 | 1072365_1 | BE141178 AW821110 BI048110 |
| 400279 | 2140_1 | Y08200 NM_004581 BC003093 BE733834 BI753321 BG773890 BF091906 |
| | | BI917541 AI023762 AA587230 BF435086 AI264262 AI687392 |
| | | AI810536 AW589886 AI244419 AA749261 AA535435 AW205689 AI765770 AI765431 C02465 AW305347 |
| | | AI818456 AA322111 AW381845 |
| | | AW381829 AV749407 AA811636 AU159893 AA603065 AA652542 AI468678 R49616 AW381863 BE389867 |
| | | BE182387 BF087771 AA527551 |
| | | AA134051 AA831504 AA134052 AI871759 AW089048 BI913532 AA367709 BG828155 BF093014 |
| 438683 | 1241644_1 | AA813982 AW978060 AA825734 |
| 415770 | 2051914_1 | AA169260 AI267298 M79237 |
| 431718 | 3517_3 | AW970730 AA524205 AW293340 AI337294 AA514491 AI858216 AA552097 AI857575 |
| 425326 | 916327_1 | BE886799 AW846391 AW501870 AW502583 AW503003 AA355226 AW751981 |
| | | AW846539 R11565 AA378251 AW846548 AW846136 |
| | | AW846484 |
| 415835 | 1894612_1 | T77496 Z45365 H05203 R25905 |
| 446780 | 1290689_1 | AL568290 AI653198 AA371134 BE005364 AI341136 BI493548 H04953 R31107 |
| 436752 | 727461_1 | AW977246 AW298529 AA972071 AA730213 |
| 455202 | 1108949_1 | AW865320 AW865316 AW865249 AW865381 AW865379 BF373916 AW865250 |
| | | AW865118 AW865123 AW865391 AW865043 BF373931 |
| | | BF373934 AW865448 |
| 419662 | 2431548_1 | W87872 W87674 W87774 |
| 412150 | 1155126_1 | AW895719 N31451 N41451 |
| 414278 | 2649_1 | BG564077 AA928199 BI491412 AI913576 AI769974 AA805536 AI978876 AI879646 |
| | | AI640283 BM153392 AA137260 AI151263 AI474103 |
| | | AW078667 AW079202 AA150376 BE048238 AI218961 AA631892 AI350813 AI758351 AI458422 AA150275 |
| | | AI038862 AW183342 AA912346 |
| | | AW468933 AI003046 AI208534 H60032 T34660 Z40410 AA621093 BE245388 N66384 AW118966 T17277 |
| | | AW020353 BE467084 AI268780 |
| | | BE003683 AA330116 R77760 AI473113 AI985726 BE813657 BE813503 BF326782 AI690850 N52225 |
| | | AW994769 BI036529 R40472 BE813513 |
| 431006 | 1524128_1 | AA490552 BE152870 BE152871 |
| 450078 | 1156200_1 | AI681743 AW897284 AW897205 AW897287 |

Pkey: Unique Eos probeset identifier number
CAT number: Gene cluster number
Accession: Genbank accession numbers

TABLE 14C

| Pkey | Ref | Strand | Nt_position |
|---|---|---|---|
| 402399 | 1905915 | Minus | 24502-24666, 24986-25102 |
| 405481 | 3688109 | Plus | 5718-5837, 8719-8818 |
| 401191 | 9716907 | Plus | 22341-22636 |
| 402885 | 9926751 | Plus | 71919-72049 |
| 405468 | 8051573 | Minus | 69367-69529, 71227-71453, |
| | | | 73076-73236 |
| 402605 | 9909420 | Minus | 47680-47973 |
| 403640 | 8671948 | Minus | 161756-161859, 164798-164914 |
| 400552 | 9801071 | Plus | 93101-93276 |
| 403609 | 8308266 | Minus | 125974-126320 |
| 401740 | 2982169 | Plus | 148357-148484, 148591-148690 |
| 405720 | 9797144 | Plus | 13409-13861 |
| 400846 | 9188605 | Plus | 39310-39474 |
| 401439 | 8246737 | Plus | 92993-94026 |
| 403740 | 7630882 | Plus | 86504-87227 |
| 402842 | 9369121 | Minus | 76355-76479 |
| 402149 | 8079589 | Minus | 86399-86618 |
| 401929 | 3810670 | Minus | 3167-3286, 4216-4310 |
| 406466 | 9795550 | Plus | 115511-115658, 118766-118915, |
| | | | 121699-12177 |
| 400636 | 3818355 | Minus | 102143-102322 |
| 405761 | 1332524 | Minus | 22866-23294 |
| 402942 | 9368398 | Plus | 102152-102386 |

Pkey: Unique number corresponding to an Eos probeset
Ref: Sequence source. The 7 digit numbers in this column are Genbank Identifier (GI) numbers. “Dunham, et al.” refers to the publication entitled “The DNA sequence of human chromosome 22” Dunham, et al. (1999) Nature 402: 489-495.
Strand: Indicates DNA strand from which exons were predicted.
Nt_position: Indicates nucleotide positions of predicted exons.

TABLE 15
Pkey: 444342
UnigeneID: Hs.10887
Unigene Title: similar to lysosome-associated membrane glycoprotein
DNA Sequence
Nucleic Acid Accession #: NM_014398.1
Coding sequence: 64..1314

| | |
|---|---|
| 1 11 21 31 41 51 | |
| \| \| \| \| \| \| | |
| GGCACCGATT CGGGGCCTGC CCGGACTTCG CCGCACGCTG CAGAACCTCG CCCAGCGCCC | 60 |
| ACCATGCCCC GGCAGCTCAG CGCGGCGGCC GCGCTCTTCG CGTCCCTGGC CGTAATTTTG | 120 |
| CACGATGGCA GTCAAATGAG AGCAAAAGCA TTTCCAGAAA CCAGAGATTA TTCTCAACCT | 180 |
| ACTGCAGCAG CAACAGTACA GGACATAAAA AAACCTGTCC AGCAACCAGC TAAGCAAGCA | 240 |
| CCTCACCAAA CTTTAGCAGC AAGATTCATG GATGGTCATA TCACCTTTCA AACAGCGGCC | 300 |
| ACAGTAAAAA TTCCAACAAC TACCCCAGCA ACTACAAAAA ACACTGCAAC CACCAGCCCA | 360 |
| ATTACCTACA CCCTGGTCAC AACCCAGGCC ACACCCAACA ACTCACACAC AGCTCCTCCA | 420 |
| GTTACTGAAG TTACAGTCGG CCCTAGCTTA GCCCCTTATT CACTGCCACC CACCATCACC | 480 |
| CCACCAGCTC ATACAGCTGG AACCAGTTCA TCAACCGTCA GCCACACAAC TGGGAACACC | 540 |
| ACTCAACCCA GTAACCAGAC CACCCTTCCA GCAACTTTAT CGATAGCACT GCACAAAAGC | 600 |
| ACAACCGGTC AGAAGCCTGA TCAACCCACC CATGCCCCAG GAACAACGGC AGCTGCCCAC | 660 |
| AATACCACCC GCACAGCTGC ACCTGCCTCC ACGGTTCCTG GGCCCACCCT TGCACCTCAG | 720 |
| CCATCGTCAG TCAAGACTGG AATTTATCAG GTTCTAAACG GAAGCAGACT CTGTATAAAA | 780 |
| GCAGAGATGG GGATACAGCT GATTGTTCAA GACAAGGAGT CGGTTTTTTC ACCTCGGAGA | 840 |
| TACTTCAACA TCGACCCCAA CGCAACGCAA GCCTCTGGGA ACTGTGGCAC CCGAAAATCC | 900 |
| AACCTTCTGT TGAATTTTCA GGGCGGATTT GTGAATCTCA CATTTACCAA GGATGAAGAA | 960 |
| TCATATTATA TCAGTGAAGT GGGAGCCTAT TTGACCGTCT CAGATCCAGA GACAGTTTAC | 1020 |
| CAAGGAATCA AACATGCGGT GGTGATGTTC CAGACAGCAG TCGGGCATTC CTTCAAGTGC | 1080 |
| GTGAGTGAAC AGAGCCTCCA GTTGTCAGCC CACCTGCAGG TGAAAACAAC CGATGTCCAA | 1140 |
| CTTCAAGCCT TTGATTTTGA AGATGACCAC TTTGGAAATG TGGATGAGTG CTCGTCTGAC | 1200 |
| TACACAATTG TGCTTCCTGT GATTGGGGCC ATCGTGGTTG GTCTCTGCCT TATGGGTATG | 1260 |
| GGTGTCTATA AAATCCGCCT AAGGTGTCAA TCATCTGGAT ACCAGAGAAT CTAATTGTTG | 1320 |
| CCCGGGGGGA ATGAAAATAA TGGAATTTAG AGAACTCTTT CATCCCTTCC AGGATGGATG | 1380 |
| TTGGGAAATT CCCTCAGAGT GTGGGTCCTT CAAACAATGT AAACCACCAT CTTCTATTCA | 1440 |
| AATGAAGTGA GTCATGTGTG ATTTAAGTTC AGGCAGCACA TCAATTTCTA AATACTTTTT | 1500 |
| GTTTATTTTA TGAAAGATAT AGTGAGCTGT TTATTTTCTA GTTTCCTTTA GAATATTTTA | 1560 |
| GCCACTCAAA GTCAACATTT GAGATATGTT GAATTAACAT AATATATGTA AAGTAGAATA | 1620 |
| AGCCTTCAAA TTATAAACCA AGGGTCAATT GTAACTAATA CTACTGTGTG TGCATTGAAG | 1680 |
| ATTTTATTTT ACCCTTGATC TTAACAAAGC CTTTGCTTTG TTATCAAATG GACTTTCAGT | 1740 |
| GCTTTTACTA TCTGTGTTTT ATGGTTTCAT GTAACATACA TATTCCTGGT GTAGCACTTA | 1800 |
| ACTCCTTTTC CACTTTAAAT TTGTTTTTGT TTTTTGAGAC GGAGTTTCAC TCTTGTCACC | 1860 |
| CAGGCTGGAG TACAGTGGCA CGATCTCGGC TTATGGCAAC CTCCGCCTCC CGGGTTCAAG | 1920 |
| TGATTCTCCT GCTTCAGCTT CCCGAGTAGC TGGGATTACA GGCACACACT ACCACGCCTG | 1980 |
| GCTAATTTTT GTATTTTTAT TATAGACGGG TTTCACCATG TTGGCCAGAC TGGTCTTGAA | 2040 |
| CTCTTGACCT CAGGTGATCC ACCCACCTCA GCCTCCCAAA GTGCTGGGAT TACAGGCATG | 2100 |
| AGCCATTGCG CCCGGCCTTA AATGTTTTTT TTAATCATCA AAAAGAACAA CATATCTCAG | 2160 |
| GTTGTCTAAG TGTTTTTATG TAAAACCAAC AAAAAGAACA AATCAGCTTA TATTTTTTAT | 2220 |
| CTTGATGACT CCTGCTCCAG AATTGCTAGA CTAAGAATTA GGTGGCTACA GATGGTAGAA | 2280 |
| CTAAACAATA AGCAAGAGAC AATAATAATG GCCCTTAATT ATTAACAAAG TGCCAGAGTC | 2340 |
| TAGGCTAAGC ACTTTATCTA TATCTCATTT CATTCTCACA ACTTATAAGT GAATGAGTAA | 2400 |
| ACTGAGACTT AAGGGAACTG AATCACTTAA ATGTCACCTG GCTAACTGAT GGCAGAGCCA | 2460 |
| GAGCTTGAAT TCATGTTGGT CTGACATCAA GGTCTTTGGT CTTCTCCCTA CACCAAGTTA | 2520 |
| CCTACAAGAA CAATGACACC ACACTCTGCC TGAAGGCTCA CACCTCATAC CAGCATACGC | 2580 |
| TCACCTTACA GGGAAATGGG TTTATCCAGG ATCATGAGAC ATTAGGGTAG ATGAAAGGAG | 2640 |
| AGCTTTGCAG ATAACAAAAT AGCCTATCCT TAATAAATCC TCCACTCTCT GGAAGGAGAC | 2700 |
| TGAGGGGCTT TGTAAAACAT TAGTCAGTTG CTCATTTTTA TGGGATTGCT TAGCTGGGCT | 2760 |
| GTAAAGATGA AGGCATCAAA TAAACTCAAA GTATTTTTAA ATTTTTTTGA TAATAGAGAA | 2820 |
| ACTTCGCTAA CCAACTGTTC TTTCTTGAGT GTATAGCCCC ATCTTGTGGT AACTTGCTGC | 2880 |
| TTCTGCACTT CATATCCATA TTTCCTATTG TTCACTTTAT TCTGTAGAGC AGCCTGCCAA | 2940 |
| GAATTTTATT TCTGCTGTTT TTTTTGCTGC TAAAGAAAGG AACTAAGTCA GGATGTTAAC | 3000 |
| AGAAAAGTCC ACATAACCCT AGAATTCTTA GTCAAGGAAT AATTCAAGTC AGCCTAGAGA | 3060 |
| CCATGTTGAC TTTCCTCATG TGTTTCCTTA TGACTCAGTA AGTTGGCAAG GTCCTGACTT | 3120 |
| TAGTCTTAAT AAAACATTGA ATTGTAGTAA AGGTTTTTGC AATAAAAACT TACTTTGG | 3178 |
| Protein Sequence | |
| Protein Accession #: NP_055213.1 | |
| 1 11 21 31 41 51 | |
| \| \| \| \| \| \| | |
| MPRQLSAAAA LFASLAVILH DGSQMRAKAF PETRDYSQPT AAATVQDIKK PVQQPAKQAP | 60 |
| HQTLAARFMD GHITFQTAAT VKIPTTTPAT TKNTATTSPI TYTLVTTQAT PNNSHTAPPV | 120 |
| TEVTVGPSLA PYSLPPTITP PAHTAGTSSS TVSHTTGNTT QPSNQTTLPA TLSIALHKST | 180 |
| TGQKPDQPTH APGTTAAAHN TTRTAAPAST VPGPTLAPQP SSVKTGIYQV LNGSRLCIKA | 240 |
| EMGIQLIVQD KESVFSPRRY FNIDPNATQA SGNCGTRKSN LLLNFQGGFV NLTFTKDEES | 300 |
| YYISEVGAYL TVSDPETVYQ GIKHAVVMFQ TAVGHSFKCV SEQSLQLSAH LQVKTTDVQL | 360 |
| QAFDFEDDHF GNVDECSSDY TIVLPVIGAI VVGLCLMGMG VYKIRLRCQS SGYQRI | 416 |

It is understood that the examples described above in no way serve to limit the true scope of this invention, but rather are presented for illustrative purposes. All publications, sequences of accession numbers, and patent applications cited in this specification are herein incorporated by reference as if each individual publication or patent application were specifically and individually indicated to be incorporated by reference.

What is claimed is:

1. A method of detecting an RNA transcript associated with Hepatitis C infection in a cell from a patient, the method comprising contacting a biological sample from the patient with a polynucleotide that selectively hybridizes to a sequence at least 80% identical to a sequence as shown in Tables 1A-15.

2. The method of claim 1, wherein:
a) the biological sample comprises isolated nucleic acids; or
b) the marker expression provides prognostic information to determine treatment alternative.

3. The method of claim 2, wherein the nucleic acids are mRNA.

4. The method of claim 2, further comprising the step of amplifying nucleic acids before the step of contacting the biological sample with the polynucleotide.

5. The method of claim 1, wherein the polynucleotide comprises a sequence as shown in Tables 1A-15.

6. The method of claim 1, wherein the polynucleotide is immobilized on a solid surface.

7. The method of claim 1, wherein the patient is undergoing a therapeutic regimen to treat Hepatitis C infection and/or its secondary consequences.

8. The method of claim 1, wherein the patient is suspected of suffering from Hepatitis C infection.

9. An isolated nucleic acid molecule consisting of a polynucleotide sequence as shown in Tables 1A-15.

10. The nucleic acid molecule of claim 9, which is labeled.

11. An expression vector comprising the nucleic acid of claim 9.

12. A host cell comprising the expression vector of claim 11.

13. An isolated polypeptide which is encoded by a nucleic acid molecule having polynucleotide sequence as shown in Tables 1A-15.

14. An antibody that specifically binds a polypeptide of claim 13.

15. The antibody of claim 14, further conjugated to an effector component.

16. The antibody of claim 15, wherein the effector component is a fluorescent label.

17. The antibody of claim 15, wherein the effector component is a radioisotope or a cytotoxic chemical.

18. The antibody of claim 15, which is an antibody fragment.

19. The antibody of claim 15, which is a humanized antibody

20. A method of detecting a cell affected by Hepatitis C infection or its secondary consequences in a biological sample from a patient, the method comprising contacting the biological sample with an antibody of claim 14.

21. The method of claim 20, wherein the antibody is further conjugated to an effector component.

22. The method of claim 21, wherein the effector component is a fluorescent label.

23. A method for identifying a compound that modulates a Hepatitis C infection-associated polypeptide, the method comprising the steps of:
a) contacting the compound with a Hepatitis C infection-associated polypeptide, the polypeptide encoded by a polynucleotide that selectively hybridizes to a sequence at least 80% identical to a sequence as shown in Tables 1A-15 and
b) determining the functional effect of the compound upon the polypeptide.

24. A drug screening assay comprising the steps of
a) administering a test compound to a mammal suffering from Hepatitis C infection and its secondary consequences or a cell isolated therefrom
b) comparing the level of gene expression of a polynucleotide that selectively hybridizes to a sequence at least 80% identical to a sequence as shown in Tables 1A-15 in a treated cell or mammal with the level of gene expression of the polynucleotide in a control cell or mammal, wherein a test compound that modulates the level of expression of the polynucleotide is a candidate for the treatment of Hepatitis C infection and its secondary consequences.
